SEQUENCE LISTING (6 sequences)

SEQ ID NO: 1 - protein, 447 aa, synthetic construct
  REGION 1..447: note = Patritumab heavy chain sequence
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 220 aa, synthetic construct
  REGION 1..220: note = Patritumab light chain sequence
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 449 aa, synthetic construct
  REGION 1..449: note = Cofetuzumab heavy chain sequence
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 220 aa, synthetic construct; identical to SEQ ID NO: 2
  REGION 1..220: note = Cofetuzumab light chain sequence

SEQ ID NO: 5 - protein, 450 aa, synthetic construct
  REGION 1..450: note = Trastuzumab heavy chain sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, synthetic construct
  REGION 1..214: note = Trastuzumab light chain sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0135026 A1
(43) Pub. Date: May 1, 2025

(54) Antibody Drug Conjugates

(71) Applicant: BeiGene Switzerland GmbH, Basel (CH)

(72) Inventors: Charng-Sheng Tsai, Shanghai (CN); Mei-Hsuan Tsai, Shanghai (CN); Xiaodong Wei, Shanghai (CN); Zewei Wang, Shanghai (CN); Wei Luo, Shanghai (CN); Ce Wang, Beijing (CN)

(73) Assignee: BeiGene Switzerland GmbH, Basel (CH)

(21) Appl. No.: 18/757,187

(22) Filed: Jun. 27, 2024

Related U.S. Application Data
(63) Continuation of application No. PCT/CN2022/142310, filed on Dec. 27, 2022.

(30) Foreign Application Priority Data
Dec. 28, 2021 (WO) PCT/CN2021/142037
Apr. 14, 2022 (WO) PCT/CN2022/086931
Apr. 24, 2022 (WO) PCT/CN2022/088762

(51) Int. Cl. A61K 47/68; A61P 35/00

(52) U.S. Cl. CPC A61K 47/6889; A61K 47/6803; A61K 47/68031; A61K 47/68037; A61K 47/6849; A61K 47/6851; A61P 35/00

(57) ABSTRACT

Antibody-drug conjugate compounds comprising a linker and methods of using such compounds are provided.

### 1. CROSS-REFERENCE TO RELATED APPLICATIONS

This application is a continuation of International Application No. PCT/CN2022/142310, filed on Dec. 27, 2022, which claims priority to International Application No. PCT/CN2022/088762, filed on Apr. 24, 2022, International Application No. PCT/CN2022/086931, filed on Apr. 14, 2022, and International Application No. PCT/CN2021/142037, filed on Dec. 28, 2021, the disclosures of each of which are hereby incorporated by reference in their entireties.

### 2. FIELD

Provided herein are novel proteins, e.g., antibody, drug conjugates comprising hydrophilic solubilizing groups and/or linkers comprising hydrophilic solubilizing groups, and methods for treating diseases, disorders, and conditions comprising administering the protein drug conjugates comprising hydrophilic solubilizing groups and/or linkers thereof.

### 3. SEQUENCE LISTING

The instant application contains a Sequence Listing, which has been submitted electronically in XML format and is hereby incorporated by reference in its entirety. Said XML copy, created on Dec. 22, 2022, is named “BGB36201-01PCT_Seql.xml” and is 10,403 bytes in size.

### 4. BACKGROUND

Antibody-drug conjugates (ADCs) are antibodies that are operably linked to a biologically active small molecule, also known as a toxin or payload. ADCs deliver a potent payload selectively to target-expressing cells, leading to a potential reduction of off-target side effects and/or toxicity and improved therapeutic index. The lipophilic nature of many payloads (i.e., drugs) can adversely affect the properties of the ADC to the extent that the payloads are not efficiently delivered to the target cells. Low bioavailability of lipophilic payloads can narrow therapeutic windows for ADC treatment. Furthermore, the hydrophobic nature of payloads can present challenges to their conjugation to antibodies, a reaction performed in aqueous conditions. Thus, there is an ongoing need for the development of hydrophilic linkers for protein conjugates, e.g., ADCs, which would allow for the feasibility of conjugating lipophilic payloads, improved modulation of biological targets, improved bioavailability, and improved therapeutic window.

Monoclonal antibody (mAb) therapies are gaining momentum as an adjunct and front-line treatments for cancer. Successes of mAb therapies like AVASTIN™ (anti-VEGF) for colon cancer, RITUXAN™ (Rituximab; anti-CD20) for Non-Hodgkin's Lymphoma and HERCEPTIN™ (anti-Her2) for breast cancer have demonstrated that unconjugated antibodies can improve patient survival without the incidence of significantly increased toxicity.

Monoclonal antibodies can be conjugated to a therapeutic agent to form an antibody-drug conjugate. For example, the HERCEPTIN™ antibody mentioned above was conjugated with a maytansine payload to form the ADC KADCYLA™. ADCs can exhibit increased efficacy, as compared to an unconjugated antibody. The linkage of the antibody to the drug can be direct, or indirect via a linker. The linker can be cleavable or non-cleavable. One of the components believed to be important for developing effective and well-tolerated ADCs is the composition and stability of the linker. For some types of ADCs, the linker desirably provides serum stability, yet selectively releases the drug within the target cell.

Attachment of a linker to a mAb can be accomplished in a variety of ways, such as through surface lysines, reductive coupling to oxidized carbohydrates, and through cysteine residues liberated by reducing interchain disulfide linkages. A variety of ADC linkage systems have been described in the literature, including hydrazone, disulfide, and peptide-based linkages. Some hydrazone and disulfide-based linkers can be labile in circulation, resulting in the undesired release of the drug outside the targeted tissue. It is believed that this premature release of drug can lead to systemic toxicity or organ-specific toxicity and/or less than optimal therapeutic efficacy. Peptide-based linker strategies can provide linkers of higher stability; however, the increased associated hydrophobicity of some linkers can lead to aggregation, particularly with strongly hydrophobic drugs. Such aggregation can lead to a number of undesired effects such as precipitation of the ADC, difficulty in administration, and non-specific uptake of the ADCs into non-targeted tissues, potentially affecting non-target toxicity and reducing efficacy.

Exatecan is a drug which is a structural analog of camptothecin with antineoplastic activity. See Abou-Alfa et al., “Randomized Phase III Study of Exatecan and Gemcitabine Compared with Gemcitabine Alone in Untreated Advanced Pancreatic Cancer,” Journal of Clinical Oncology, 24 (27): 4441-7, Sep. 20, 2006. Monomethyl auristatin E (MMAE) is a synthetic antineoplastic agent. Because of its toxicity, it cannot be used as a drug itself. MMAE is actually desmethyl-auristatin E; that is, the N-terminal amino group has only one methyl substituent instead of two as in auristatin E itself. See Dosio et al., “Immunotoxins and Anticancer Drug Conjugate Assemblies: The Role of the Linkage between Components,” Toxins. 3 (12): 848-883, 2011.

In the realm of small molecule therapeutics, strategies have been developed to provide prodrugs of an active chemical entity. Such prodrugs are administered in a relatively inactive (or significantly less active) form. Once administered, the prodrug is metabolized in vivo into the active compound. Such prodrug strategies can provide for increased selectivity of the drug for its intended target and for a reduction of adverse effects.

There remains a need, therefore, for targeted delivery of toxins, resulting in the elimination of targeted cells while reducing toxicity to non-target cells.

Furthermore, some antibody, such as Patritumab, has visually observable aggregation during rapid buffer exchange. Its aggregation temperature (Tagg) detected by dynamic light scattering (DLS) and self-association tendency detected by the AC-SINS assay are both worse than those of a panel of well-behaved mAbs. The aggregation tendency of patritumab results in the aggregation of the corresponding ADCs.

There is therefore unmet medical need to create ADCs with linker systems that provide a high level of linker serum stability and increased solubility, allowing the efficient conjugation of hydrophobic drugs and that effect intracellular delivery of drugs.

### 5. BRIEF SUMMARY

Provided herein are ADCs with linker systems that provide a high level of linker serum stability and increased solubility, allowing the efficient conjugation of hydrophobic drugs and that effect intracellular delivery of drugs. In one embodiment, the ADC is a compound of Formula (I):

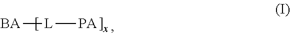

or a pharmaceutically acceptable salt tautomer, solvate, stereoisomer, enantiomer, isotopologue, or prodrug thereof, wherein the variables are defined as herein. In one embodiment, the ADC comprises one or more hydrophilic residues.

In another embodiment, the ADC is a compound of Formula (Ia):

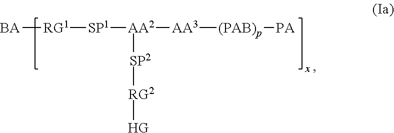

or a pharmaceutically acceptable salt tautomer, solvate, stereoisomer, enantiomer, isotopologue, or prodrug thereof, wherein the variables are defined as herein.

In another embodiment, the ADC is a compound of Formula (Ib):

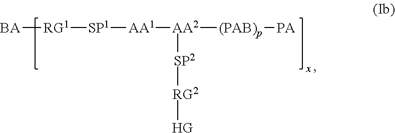

or a pharmaceutically acceptable salt tautomer, solvate, stereoisomer, enantiomer, isotopologue, or prodrug thereof, wherein the variables are defined as herein.

In another embodiment, set forth herein is a method of treating a disease, condition, or disorder in a patient in need thereof including administering to the patient a compound set forth herein. Also provided is the use of a compound set forth herein for treating a disease, condition, or disorder set forth herein. Further provided is the use of a compound set forth herein for the manufacture of a medicament for treating a disease, condition, or disorder set forth herein. In some embodiments, the compound is an antibody-drug conjugate.

In another embodiment, set forth herein is a method of preparing an antibody-drug conjugate including the step of contacting a binding agent with a linker-payload compound set forth herein under conditions suitable for forming a bond between the binding agent and the linker-payload compound.

### 6. BRIEF DESCRIPTION OF FIGURES

FIG. 1 illustrates the Human Plasma stability profiles of ADC2-B. ADC2-B_Tab curve indicates the concentration ratios of the antibody at different time compared with its concentration at TO points (so called total antibody curve). It is included both non-payload-conjugated and payload-conjugated antibody, which is captured by antigen (HER2) from plasma. ADC2-B_ADC curve indicates the concentration ratios of the drug-conjugated antibody ADC2-B at different time points compared with its concentration at TO (so called ADC curve). ADC2-B_Payload curve indicates the payload release ratio at different time points compared with its concentration at TO (so called payload release curve). At time point 168 h, Dxd release rate of ADC2-B is less than 5%. ADC2-B was not undergone maleimide ring open process.

FIG. 2 illustrates the Human Plasma stability profiles of ADC2-40. ADC2-40_Tab is the total antibody curve. It is included both non-payload-conjugated and payload-conjugated antibody, which is captured by antigen (HER2) from plasma. ADC2-40_ADC is the ADC curve. ADC2-40_Payload curve is the payload release curve. ADC2-40 was undergone maleimide ring open process (the ring opening) and has the P5 modification. From the payload release curve, it has shown less free payload release (<2%) than ADC2-B indicating the good linker stability. Moreover, with maleimide ring open, ADC curve is almost overlapped with Tab curve indicating that less deconjugation event was occurred.

FIG. 3 illustrates the Human Plasma stability profiles of ADC2-32. ADC2-32_Tab curve is the total antibody curve. ADC2-32_ADC curve is the ADC curve. ADC2-32 Payload curve is the payload release curve. ADC2-32 does not have the ring opening, but has the P5 modification. ADC2-32 at timepoint T168 h has shown less payload release rate (<2%) than ADC2-B.

FIG. 4 illustrates the Human Plasma stability profiles of ADC2-42. ADC2-42_Tab curve is the total antibody curve. ADC2-42_ADC curve is the ADC curve. ADC2-42 Payload curve is the payload release curve. ADC2-42 has the ring opening and P5 modification. ADC2-42 is similar with ADC2-40 and has shown that ADC and Tab curves are almost overlapped, and payload release rate (ex. T168 h) is less than ADC2-B. FIG. 2, and FIG. 4 illustrate that the antibody curves and the ADC curves almost overlap with each other, respectively, which demonstrates that the human plasma stability of ADC2-40 and ADC2-42 is consistent with that of the corresponding antibodies.

FIG. 5 illustrates the Human Plasma stability profiles of ADC2-B, ADC2-32, and ADC2-40, and ADC2-42 showing the corresponding payload release curves. ADC2-B does not have its ring opened. ADC2-32 does not have its ring opened, but contains the corresponding P5 modification. ADC2-40 and ADC2-42 have their rings opened, and contain the corresponding P5 modification. FIG. 5 demonstrates that the ADCs with their maleimide rings opened have their free payload release lower than the ADCs with no ring opening treatment in human plasma. With P5 modification, ADC 2-32 has shown less payload release than ADC2-B.

FIG. 6 illustrates the Rat PK profiles based on the concentration curves of total antibodies (including the antibodies with and without payload-conjugate) of ADC2-B, ADC2-32, ADC2-40, and ADC2-42. ADC2-B and ADC2-32 do not have their rings opened, but contain the corresponding P5 modification. ADC2-40_Tab and ADC2-42_Tab have their rings opened, and contain the corresponding P5 modification. The Rat PK profiles based on the concentration of the antibodies are similar for the ADCs with or without ring-opening.

FIG. 7 illustrates the Rat PK profiles based on the concentration curves of the ADC (payload-conjugated antibody) of ADC2-B, ADC2-32, ADC2-40, and ADC2-42. ADC2-B and ADC2-32 do not have their ring opened, but contain the corresponding P5 modification. ADC2-40 and ADC2-42 have their rings opened, and contain the corresponding P5 modification. The Rat PK profiles based on the concentration of the ADCs indicate that the ADCs with ring-opening clear slower than the ADCs without ring-opening.

FIG. 8 illustrates the Rat PK profiles of ADC2-B. ADC2-B_ADC curve indicates the concentration of the ADC (payload-conjugated antibody). ADC2-B_Tab total antibody curve indicates the concentration of the total antibody including the non-payload-conjugated and payload-linked ones. ADC2-B does not have its ring opened.

FIG. 9 illustrates the Rat PK profiles of ADC2-40. ADC2-40_ADC curve indicates the concentration of the ADC (payload-conjugated antibody). ADC2-40_Tab is the total antibody curve that indicates the concentration of the antibody including the non-payload-conjugated and payload-linked ones. ADC2-40 has its ring opened, but contains the corresponding P5 modification. FIG. 9 illustrates that the curves of ADC2-40_ADC, and ADC2-40_Tab almost overlap with each other, which demonstrates that the PK of the ring-opened ADC is driven by the PK of the corresponding antibodies.

FIG. 10 illustrates the Rat PK profiles of ADC2-32. ADC2-32_ADC curve indicates the concentration of the ADC (payload-conjugated antibody). ADC2-32_Tab curve indicates the concentration of the antibody including the non-payload-conjugated and payload-linked ones. ADC2-32 does not have its ring opened, but contains the corresponding P5 modification.

FIG. 11 illustrates the Rat PK profiles of ADC2-42. ADC2-42_ADC curve indicates the concentration of the ADC (payload-conjugated antibody). ADC2-42_Tab curve indicates the concentration of the antibody including the non-payload-conjugated and payload-linked ones. ADC2-42 has its rings opened, and contains the corresponding P5 modification. FIG. 11 illustrates that the curves of ADC2-42_ADC, and ADC2-42_Tab almost overlap with each other, which demonstrates that the PK of the ring-opened ADC is driven by the PK of the corresponding antibodies.

FIG. 12 illustrates the MMAE release percentage in human plasma stability study of ADC2-A, ADC2-2, ADC2-4, ADC2-11, and ADC2-9-1. ADC2-A contains no P3 modification, but ADC2-2, ADC2-4, ADC2-11, and ADC2-9-1 contain the P3 modification.

FIG. 13 illustrates the MMAE release percentage in mouse plasma stability study of ADC2-A, ADC2-2, ADC2-4, ADC2-11, and ADC2-9-1. ADC2-A contains no P3 modification, but ADC2-2, ADC2-4, ADC2-11, and ADC2-9-1 contain the P3 modification. FIG. 12 and FIG. 13 demonstrate that the P3 modification of MMAE ADC can reduce the cleavage of the VC-PAB linker by mouse system Ces1C enzyme, i.e., reducing pre-mature free MMAE release in the mouse system, at least because the MMAE ADC with P3 modification, e.g., ADC2-2, ADC2-4, ADC2-11, and ADC2-9-1, showed reduced pre-mature free MMAE release.

FIG. 14 illustrates the mouse PK profiles of ADC2-A, ADC2-2, ADC2-4, ADC2-11, and ADC2-9-1. ADC2-A contains no P3 modification, but ADC2-2, ADC2-4, ADC2-11, and ADC2-9-1 contain the P3 modification. FIG. 14 demonstrates that the P3 modification of MMAE ADC, e.g., ADC2-2, ADC2-4, ADC2-11, and ADC2-9-1, leads to reduced pre-mature free MMAE release in the mouse system, compared to the MMAE ADC without P3 modification, e.g., ADC2-A. In other words, FIG. 14 demonstrates that the MMAE ADC without P3 modification, i.e., ADC2-A, has more premature MMAE release than the MMAE ADCs with P3 modification, e.g., ADC2-2, ADC2-4, ADC2-11, and ADC2-9-1.

FIG. 15 illustrates ADC 2-6-2 Hydrophobic interaction chromatography (HIC) profile (HIC DAR 8.0).

FIG. 16 illustrates ADC 2-6-2 size exclusion chromatography (SEC) profile with >98% purity.

FIG. 17 illustrates ADC 2-9-2 HIC profile (HIC DAR 8.0).

FIG. 18 illustrates ADC 2-9-2 SEC profile (>99% purity).

FIG. 19 illustrates ADC2-36-2 HIC profile (HIC DAR7.8).

FIG. 20 illustrates ADC2-36-2 SEC profile with >99% purity.

FIG. 21 illustrates different cathepsin B mediated rhodamine release rates. The rate ranks as follow: NAcCys-15˜NAcCys-13 (benchmark)>NAcCys-14>NAcCys-17.

FIG. 22 illustrates the inhibition of the Ovcar3 cell line.

FIG. 23 illustrates the inhibition of the A549 cell line.

FIG. 24 and FIG. 25 illustrate the data of the NCI-N87 cell viability detection.

FIG. 26 illustrates the data for the NCI-N87 cell viability detection.

FIG. 27 and FIG. 28 illustrate the data for the NCI-N87 cell viability detection.

FIG. 29 and FIG. 30 illustrate the data for the NCI-N87 cell viability detection.

FIG. 31 and FIG. 32 illustrate the data for the NCI-N87 cell viability detection.

FIG. 33 describes Payload release % of ADCs in human plasma

FIG. 34 describes Free MMAE release in Mouse PK study.

FIG. 35 illustrates ADC DAR changes of ADC2-B, ADC2-32, and ADC2-42 in human plasma.

FIG. 36 illustrates ADC DAR changes of ADC2-B and ADC2-42 in human plasma.

FIG. 37 illustrates ADC DAR changes of ADC2-B, ADC2-32, and ADC2-40, and ADC2-42 in rat PK.

FIG. 38 illustrates the HCC1569 cellular killing comparison between P5 modified ADCs, (i.e., ADC3-2, ADC3-3, ADC3-4, ADC3-6) and ADC3-1 in part A and between P5 modified ADCs (i.e., ADC3-5, ADC3-7, ADC3-8, ADC3-9, ADC3-10) and ADC3-1 in part B.

FIG. 39 illustrates the HCC1569 cellular killing comparison between P3 modified ADCs, (i.e., ADC3-12, and ADC3-15) and ADC3-1 in part A and between P3 modified ADCs (i.e., ADC3-11, and ADC3-14) and ADC3-1 in part B.

FIG. 40 illustrates the human plasma stability profiles of ADC3-1, ADC3-2, ADC3-3, ADC3-4, ADC3-5, ADC3-6, ADC3-7, ADC3-8, ADC3-9, and ADC3-10 as measured by the concentration of the corresponding antibodies. ADC3-1_TAB, ADC3-2_TAB, ADC3-3_TAB, ADC3-4_TAB, ADC3-5_TAB, ADC3-6_TAB, ADC3-7_TAB, ADC3-8_TAB, ADC3-9_TAB, and ADC3-10_TAB are the total antibody curves that indicate the concentration ratios of the antibodies at different time compared with their concentration at TO points, respectively, including the non-payload-conjugated and payload-linked antibodies. This figure illustrates that the total antibody curves of the corresponding P5-modified ring-opened ADCs almost overlap with the ADC curves of P5-modified non-ring-opened ADCs, which demonstrates that the human plasma stability of the P5-modified ring-opened ADCs is consistent with those of the P5-modified non-ring-opened ADCs as measured by the concentration of the corresponding antibodies.

FIG. 41 illustrates the human plasma stability profiles of ADC3-1, ADC3-2, ADC3-3, ADC3-4, ADC3-5, ADC3-6, ADC3-7, ADC3-8, ADC3-9, and ADC3-10 as measured by the concentration of the corresponding ADCs. ADC3-1_ADC, ADC3-2_ADC, ADC3-3_ADC, ADC3-4_ADC, ADC3-5_ADC, ADC3-6_ADC, ADC3-7_ADC, ADC3-8_ADC, ADC3-9_ADC, and ADC3-10_ADC are the ADC curves that indicate the concentration ratios of the corresponding drug-conjugated antibody at different time points compared with its concentration at TO. This figure illustrates that the P5-modified ring-opened ADCs (ADC3-4, ADC3-5, ADC3-6, ADC3-7, ADC3-8) have better stability than P5-modified non-ring-opened ADCs. However, the stability of P5-modified ring-opened ADCs is similar. The stability of P5-modified non-ring-opened ADCs is similar as well.

FIG. 42 illustrates the human plasma stability profiles of ADC3-1, ADC3-2, ADC3-3, ADC3-4, ADC3-5, ADC3-6, ADC3-7, ADC3-8, ADC3-9, and ADC3-10 as measured by the free payload release. ADC3-1_ADC_Payload, ADC3-2_ADC_Payload, ADC3-3_ADC_Payload, ADC3-4_ADC_Payload, ADC3-5_ADC_Payload, ADC3-6_ADC_Payload, ADC3-7_ADC_Payload, ADC3-8_ADC_Payload, ADC3-9_ADC_Payload, and ADC3-10_ADC_Payload curves are the payload release curves that indicate the payload release ratios at different time points compared with their concentrations at TO. This figure illustrates that the concentration of the free payload of all the ADCs is less than 3% after 168 hours in human plasma, which demonstrates that the human plasma stability of the ADCs is within the acceptable range. The ring-opened ADCs released even less payload than the non-ring-opened ADCs.

FIG. 43 illustrates the human plasma stability profiles of ADC3-1, ADC3-11, ADC3-12, ADC3-13, ADC3-14, and ADC3-15 as measured by the concentration of the corresponding antibodies. ADC3-1_TAB, ADC3-11_TAB, ADC3-12_TAB, ADC3-13_TAB, ADC3-14_TAB, and ADC3-15_TAB curves are the total antibody curves that indicate the concentration ratios of the antibodies at different time compared with their concentration at TO points, respectively, including the non-payload-conjugated and payload-linked antibodies. This figure illustrates that the total antibody curves of the corresponding P3-modified ADCs almost overlap with each other, which demonstrates that the human plasma stability of the P3-modified ADCs is similar as measured by the concentration of the corresponding antibodies.

FIG. 44 illustrates the human plasma stability profiles of ADC3-1, ADC3-11, ADC3-12, ADC3-13, ADC3-14, and ADC3-15 as measured by the concentration of the corresponding ADCs. ADC3-1_ADC, ADC3-11_ADC, ADC3-12_ADC, ADC3-13_ADC, ADC3-14_ADC, and ADC3-15_ADC curves are the ADC curves. This figure illustrates that the P3-modified ring-opened ADCs (ADC3-11, ADC3-12, and ADC3-13) have better stability than P3-modified non-ring-opened ADCs. However, the stability of P3-modified ring-opened ADCs is similar. The stability of P3-modified non-ring-opened ADCs is similar as well.

FIG. 45 illustrates the human plasma stability profiles of ADC3-1, ADC3-11, ADC3-12, ADC3-13, ADC3-14, and ADC3-15 as measured by the free payload release. ADC3-1_ADC_Payload, ADC3-11_ADC_Payload, ADC3-12_ADC_Payload, ADC3-13_ADC_Payload, ADC3-14_ADC_Payload, and ADC3-15_ADC_Payload curves are the payload release curves that indicate the concentration of the released free payload of the corresponding ADCs. This figure illustrates that the concentration of the free payload of all the ADCs is less than 3% after 168 hours in human plasma, which demonstrates that the human plasma stability of the ADCs is within the acceptable range. The ring-opened ADCs released even less payload than the non-ring-opened ADCs.

FIG. 46 illustrates the study results of ADC3-1, ADC3-3, and ADC3-4 on the xenograft tumor model in which P5-modified non-ring-opened ADC (ADC3-3), and P5-modified ring-opened ADC (ADC3-4) showed similar efficacy with ADC3-1.

FIG. 47 illustrates the study results of ADC3-1, ADC3-3, and ADC3-4 on the xenograft tumor model in which P5-modified non-ring-opened ADC (ADC3-3), P5-modified ring-opened ADC (ADC3-4) showed similar efficacy with ADC3-1.

FIG. 48 illustrates the study results of ADC3-1, and ADC3-7 on the A375 xenograft tumor model in which P5-modified ring-opened ADC (ADC3-7) showed similar efficacy with ADC3-1.

FIG. 49 illustrates the study results of ADC3-1, and ADC3-7 on the A375 xenograft tumor model in which P5-modified ring-opened ADC (ADC3-7) showed similar efficacy with ADC3-1.

FIG. 50 illustrates the study results of ADC3-1, ADC3-12, and ADC3-15 on the A375 xenograft tumor model in which P3-modified non-ring-opened ADC (ADC3-15), P5-modified ring-opened ADC (ADC3-12) showed similar efficacy with ADC3-1.

FIG. 51 illustrates the study results of ADC3-1, ADC3-12, and ADC3-15 on the A375 xenograft tumor model in which P3-modified non-ring-opened ADC (ADC3-15), P5-modified ring-opened ADC (ADC3-12) showed better efficacy with ADC3-1. In particular, 1/8 CR and 2/8 PR were observed for ADC3-12.

FIG. 52 illustrates the study results of ADC3-1, ADC3-3 and ADC3-4 on the A375 xenograft tumor model. The results demonstrate that P5-modified non-ring-opened ADC (ADC3-3), and P5-modified ring-opened ADC (ADC3-4) showed similar pharmacokinetic profiles with the reference ADC3-1.

FIG. 53 illustrates the study results of ADC3-1, ADC3-7, ADC3-12, and ADC3-15 in the mice pharmacokinetic study in which P5-modified ring-opened ADC (ADC3-7), P5-modified ring-opened ADC (ADC3-12), and P3-modified non-ring-opened ADC (ADC3-15) showed similar pharmacokinetic profiles with ADC3-1. This demonstrated that the ADC efficacy is positively correlated with the ADC pharmacokinetic profiles.

FIG. 54 illustrates that ADC2-62-1 and ADC2-62-2 have comparable cell-killing activity compared to the reference ADC (ADC2-A with DAR4) in JIMT-1 (HER2 moderate expression level).

FIG. 55 illustrates that ADC2-62-2 with DAR8 has better cell-killing activity compared to the reference ADC (ADC2-A with DAR4) in Capan-1 (HER2 low expression level).

FIG. 56 illustrates that ADC2-63-1 (DAR4) has comparable cell-killing activity compared to the reference ADC (ADC2-A with DAR4) in JIMT-1 (HER2 moderate expression level), and that ADC2-63-2 (DAR8) have better cell-killing activity to the reference ADC in JIMT-1 (HER2 moderate expression level).

FIG. 57 illustrates that ADC2-63-1 (DAR4) has comparable cell-killing activity compared to the reference ADC (ADC2-A with DAR4) in Capan-1 (HER2 low expression level), and that ADC2-63-2 (DAR8) have better cell-killing activity to the reference ADC in Capan-1(HER2 low expression level).

FIG. 58 illustrates that ADC2-64-1 with DAR4 has comparable cell-killing activity to the reference ADC (ADC2-A with DAR4) in JIMT-1 (HER2 moderate expression level) and that ADC2-64-2 with DAR8 has better cell-killing activity to the reference ADC (ADC2-A with DAR4) in JIMT-1 (HER2 moderate expression level).

FIG. 59 illustrates that ADC2-64-1(DAR4) has comparable cell-killing activity to the reference ADC (ADC2-A with DAR4) in Capan-1(HER2 low expression level) and that ADC2-64-2 with DAR8 has better cell-killing activity to the reference ADC (ADC2-A with DAR4) in Capan-1(HER2 low expression level).

FIG. 60 illustrates that ADC2-65-1 with DAR4 has comparable cell-killing activity to the reference ADC (ADC2-A with DAR4) in JIMT-1 (HER2 moderate expression level), and that ADC2-65-2 with DAR8 has better cell-killing activity to the reference ADC (ADC2-A with DAR4) in JIMT-1 (HER2 moderate expression level).

FIG. 61 illustrates that ADC2-65-1(DAR4) has comparable cell-killing activity to the reference ADC (ADC2-A with DAR4) in Capan-1(HER2 low expression level), and that ADC2-65-2 with DAR8 has better cell-killing activity to the reference ADC (ADC2-A with DAR4) in Capan-1(HER2 low expression level).

FIG. 62 illustrates that ADC2-62-2 HIC profile (HIC DAR 7.7).

FIG. 63 illustrates that ADC2-62-2 SEC profile with >99% purity.

FIG. 64 illustrates that ADC2-63-2 HIC profile (HIC DAR 8.0).

FIG. 65 illustrates that ADC2-63-2 SEC profile with >99% purity.

FIG. 66 illustrates that ADC2-64-2 HIC profile (HIC DAR 7.7).

FIG. 67 illustrates that ADC2-64-2 SEC profile with >99% purity.

FIG. 68 illustrates that ADC2-65-2 HIC profile (HIC DAR 7.5).

FIG. 69 illustrates that ADC2-65-2 SEC profile with >99% purity.

FIG. 70 and FIG. 71 illustrate that Example 66 & Example 69 showed stronger cell-killing activity than Dxd in A375 and Calu-6 cancer cell lines and that Example 68 & Example 72 were less potent than Dxd in A375 and Calu-6 cancer cell lines.

FIG. 72, FIG. 73, and FIG. 74 illustrate that ADC-C2 had stronger killing potency in MDA-MB-453 than ADC-C1, and that ADC-C3 & ADC-C5 showed better cell-killing activity in A375 than ADC-C1.

FIG. 75, FIG. 76, and FIG. 77 illustrate that ADC-C3 had stronger bystander cell-killing activity in MDA-MB-453 & Calu-6-nanoLuc system than ADC-C1, and that ADC-C3 & ADC-C5 showed better bystander killing potency in A375 & Calu-6-nanoLuc system than ADC-C1.

FIG. 78 depicts the sequences of the antibodies used herein.

FIG. 79 depicts the in vivo efficacy of ADC-C1, ADC-C3, and ADC-C5 compared with A-375 melanoma xenografts grown subcutaneously in BALB/c nude mice.

FIG. 80 depicts the in vivo efficacy of ADC-C1, and ADC-C2 were compared in A-375 melanoma xenografts grown subcutaneously in BALB/c nude mice.

### 7. DETAILED DESCRIPTION

Provided herein are compounds, compositions, ADCs, and methods useful for treating a wide variety of human cancers, including, but not limited to, colorectal cancer, gastric cancer, breast cancer, non-small cell lung cancer (NSCLC), ovarian cancer, head and neck cancer, pancreatic cancer and cervical cancer. In one embodiment, provided herein are compounds, compositions, ADCs, and methods useful for treating a wide variety of human cancers.

Within the present disclosure, it is understood that the disclosure does not limit to particular methods and/or experimental conditions described, as such methods and conditions may vary. It is also to be understood that the terminology used herein is for the purpose of describing particular embodiments only, and is not intended to be limiting,

Although any methods and materials similar or equivalent to those described herein can be used in the practice or testing of the present invention, the preferred methods and materials are now described. All patents, applications and non-patent publications mentioned in this specification are incorporated herein by reference in their entireties.

### 7.1. Definitions

When referring to the compounds provided herein, the following terms have the following meanings unless indicated otherwise. Unless defined otherwise, all technical and scientific terms used herein have the same meaning as is commonly understood by one of ordinary skill in the art to which this invention belongs. In the event that there is a plurality of definitions for a term provided herein, these Definitions prevail unless stated otherwise.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with amounts, or weight percentage of ingredients of a composition, mean an amount, or weight percent that is recognized by one of ordinary skill in the art to provide a pharmacological effect equivalent to that obtained from the specified amount, or weight percent. In certain embodiments, the terms “about” and “approximately,” when used in this context, contemplate an amount, or weight percent within 30%, within 20%, within 15%, within 10%, or within 5%, of the specified amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), singlecrystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2°2θ (or ±0.2 degree 2θ) while still describing the particular XRPD peak.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and n-hexyl; while saturated branched alkyls include -isopropyl, -sec-butyl, -isobutyl, -tert-butyl, -isopentyl, 2-methylpentyl, 3methylpentyl, 4-methylpentyl, 2,3-dimethylbutyl and the like. Examples of unsaturated alkyl groups include, but are not limited to, vinyl, allyl, CH═CH(CH₃), —CH═C(CH₃)₂, —C(CH₃)═CH₂, —C(CH₃)═CH(CH₃), C(CH₂CH₃)═CH₂, C≡CH, —C≡C(CH₃), —C≡C(CH₂CH₃), —CH₂C≡CH, —CH₂C≡C(CH₃) and CH₂C≡C(CH₂CH₃), among others. An alkyl group can be substituted or unsubstituted. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂C₈)alkenyls include -vinyl, -allyl, -1-butenyl, -2-butenyl, -isobutylenyl, -1-pentenyl, 2pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, 3octenyl and the like. The double bond of an alkenyl group can be unconjugated or conjugated to another unsaturated group. An alkenyl group can be unsubstituted or substituted.

A “cycloalkyl” group is a saturated, or a partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, thiazolyl, pyrrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3Himidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated and saturated ring systems, such as, for example, imidazolyl, imidazolinyl and imidazolidinyl groups. The phrase heterocyclyl includes fused ring species, including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridin-2(3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

A “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocyclylalkyl groups include but are not limited to 4-ethyl-morpholinyl, 4-propylmorpholinyl, furan-2-yl methyl, furan-3-yl methyl, pyrdine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

A “hydroxyalkyl” group is an alkyl group as described above substituted with one or more hydroxy groups.

An “alkoxy” group is O(alkyl), wherein alkyl is defined above.

An “alkoxyalkyl” group is (alkyl)O(alkyl), wherein alkyl is defined above.

An “amine” group is a radical of the formula: NH₂.

A “hydroxyl amine” group is a radical of the formula: N(R^{#})OH or NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula: —N(R^{#})O-alkyl or —NHO-alkyl, wherein R^{#} is as defined above.

An “aralkoxyamine” group is a radical of the formula: N(R^{#})O-aryl or NHOaryl, wherein R^{#} is as defined above.

An “alkylamine” group is a radical of the formula: NHalkyl or N(alkyl)₂, wherein each alkyl is independently as defined above.

An “aminocarbonyl” group is a radical of the formula: —C(═O)N(R^{#})₂, —C(═O)NH(R^{#}), or C(═O)NH₂, wherein each R^{#} is as defined above.

An “acylamino” group is a radical of the formula: NHC(═O)(R^{#}) or N(alkyl)C(═O)(R^{#}), wherein each alkyl and R^{#} are independently as defined above.

An “O(alkyl)aminocarbonyl” group is a radical of the formula: —O(alkyl)C(═O)N(R^{#})₂, —O(alkyl)C(═O)NH(R^{#}) or —O(alkyl)C(═O)NH₂, wherein each R^{#} is independently as defined above.

An “N-oxide” group is a radical of the formula: —N⁺—O⁻.

A “carboxy” group is a radical of the formula: C(═O)OH.

A “ketone” group is a radical of the formula: C(═O)(R^{#}), wherein R^{#} is as defined above.

An “aldehyde” group is a radical of the formula: —CH(═O).

An “ester” group is a radical of the formula: C(═O)O(R^{#}) or OC(═O)(R^{#}), wherein R^{#} is as defined above.

A “urea” group is a radical of the formula: —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or NHC(═O)NH₂^{#}, wherein each alkyl and R^{#} are independently as defined above.

An “imine” group is a radical of the formula: —N═C(R^{#})₂ or —C(R^{#})═N(R^{#}), wherein each R^{#} is independently as defined above.

An “imide” group is a radical of the formula: —C(═O)N(R#)C(═O)(R^{#}) or N((C═O)(R^{#}))₂, wherein each R^{#} is independently as defined above.

A “urethane” group is a radical of the formula: —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula: —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})═NH, or —NHC(R^{#})═NH, wherein each R^{#} is independently as defined above.

A “guanidine” group is a radical of the formula: —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R^{#})₂)₂, —N═C(NH(R^{#}))₂, or —N═C(NH₂)₂, wherein each R^{#} is independently as defined above.

An “enamine” group is a radical of the formula: —N(R^{#})C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R^{#})₂, —C(R^{#})═C(R^{#})(N(R^{#})₂), C(R^{#})═C(R^{#})(NH(R^{#})) or —C(R^{#})═C(R^{#})(NH₂), wherein each R^{#} is independently as defined above.

An “oxime” group is a radical of the formula: —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula: —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula: —N(R^{#})N(R^{#})₂, —NHN(R^{#})₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula: —C(═N—N(R^{#})₂)(R^{#})₂, —C(═NNH(R^{#}))(R^{#})₂, —C(═N—NH₂)(R^{#})₂, —N(R^{#})(N═C(R^{#})₂), or —NH(N═C(R^{#})₂), wherein each R^{#} is independently as defined above.

An “azide” group is a radical of the formula: —N₃.

An “isocyanate” group is a radical of the formula: N═C═O.

An “isothiocyanate” group is a radical of the formula: N═C═S.

A “cyanate” group is a radical of the formula: OCN.

A “thiocyanate” group is a radical of the formula: SCN.

A “thioether” group is a radical of the formula; —S(R^{#}), wherein R is as defined above.

A “thiocarbonyl” group is a radical of the formula: —C(═S)(R^{#}), wherein R^{#} is as defined above.

A “sulfinyl” group is a radical of the formula: —S(═O)(R^{#}), wherein R is as defined above.

A “sulfone” group is a radical of the formula: —S(═O)₂(R^{#}), wherein R is as defined above.

A “sulfonylamino” group is a radical of the formula: —NHSO₂(R^{#}) or —N(alkyl)SO₂(R^{#}), wherein each alkyl and R^{#} are defined above.

A “sulfonamide” group is a radical of the formula: —S(═O)₂N(R^{#})₂, or —S(═O)₂NH(R^{#}), or —S(═O)₂NH₂, wherein each R^{#} is independently as defined above.

A “phosphonate” group is a radical of the formula: —P(═O)(O(R^{#}))₂, —P(═O)(OH)₂, —OP(═O)(O(R^{#}))(R^{#}), or —OP(═O)(OH)(R^{#}), wherein each R^{#} is independently as defined above.

A “phosphine” group is a radical of the formula: —P(R^{#})₂, wherein each R^{#} is independently as defined above.

When the groups described herein, with the exception of alkyl group are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base.

As used herein and unless otherwise indicated, the term “clathrate” means a compound, or a salt thereof, in the form of a crystal lattice that contains spaces (e.g., channels) that have a guest molecule (e.g., a solvent or water) trapped within or a crystal lattice wherein a compound is a guest molecule.

As used herein and unless otherwise indicated, the term “solvate” means a compound, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a compound, or a salt thereof, that further includes a stoichiometric or non-stoichiometric amount of water bound by non-covalent intermolecular forces.

As used herein and unless otherwise indicated, the term “prodrug” means a compound derivative that can hydrolyze, oxidize, or otherwise react under biological conditions (in vitro or in vivo) to provide an active compound, particularly a compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (WileyInterscience, New York, 1981); Wilen, S. H., et al., Tetrahedron 33:2725 (1977); Eliel, E. L., Stereochemistry of Carbon Compounds (McGrawHill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the cis or trans isomer. In other embodiments, the compounds are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in an aqueous solution, pyrazoles may exhibit the following isomeric forms, which are referred to as tautomers of each other:

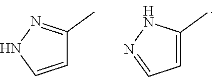

As readily understood by one skilled in the art, a wide variety of functional groups and other structures may exhibit tautomerism and all tautomers of the compounds are within the scope of the present invention.

It should also be noted the compounds can contain unnatural proportions of atomic isotopes at one or more of the atoms. For example, the compounds may be radiolabeled with radioactive isotopes, such as for example tritium (³H), iodine-125 (¹²⁵I), sulfur35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

It should be noted that if there is a discrepancy between a depicted structure and a name for that structure, the depicted structure is to be accorded more weight.

The term “effective amount” in connection with a compound means an amount capable of alleviating, in whole or in part, symptoms, or slowing or halting further progression or worsening of those symptoms. As will be apparent to those skilled in the art, it is to be expected that the effective amount of a compound disclosed herein may vary depending on the severity of the indication being treated.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

As used herein, “haloalkyl” refers to alkyl, as defined above, wherein the alkyl includes at least one substituent selected from a halogen, for example, fluorine (F), chlorine (Cl), bromine (Br), or iodine (I). Examples of haloalkyl include, but are not limited to, —CF₃, —CH₂CF₃, —CCl₂F, and —CCl₃.

As used herein, “haloalkoxy” refers to alkoxy, as defined above, wherein the alkoxy includes at least one substituent selected from a halogen, e.g., F, Cl, Br, or I.

As used herein, “arylalkyl” refers to a monovalent moiety that is a radical of an alkyl compound, wherein the alkyl compound is substituted with an aromatic substituent, i.e., the aromatic compound includes a single bond to an alkyl group and wherein the radical is localized on the alkyl group. An arylalkyl group bonds to the illustrated chemical structure via the alkyl group. An arylalkyl can be represented by the structure, e.g., B—CH₂—, B—CH₂—CH₂—, B—CH₂—CH₂—CH₂—, B—CH₂—CH₂—CH₂—CH₂—, B—CH(CH₃)—CH₂—CH₂—, B—CH₂—CH(CH₃)—CH₂—, wherein B is an aromatic moiety, e.g., phenyl. Arylalkyl is optionally substituted, i.e., the aryl group and/or the alkyl group, can be substituted as disclosed herein. Examples of arylalkyl include, but are not limited to, benzyl.

As used herein, “alkylaryl” refers to a monovalent moiety that is a radical of an aryl compound, wherein the aryl compound is substituted with an alkyl substituent, i.e., the aryl compound includes a single bond to an alkyl group and wherein the radical is localized on the aryl group. An alkylaryl group bonds to the illustrated chemical structure via the aryl group. An alkylaryl can be represented by the structure, e.g., —B—CH₃, —B—CH₂—CH₃, —B—CH₂—CH₂—CH₃, —B—CH₂—CH₂—CH₂—CH₃, —B—CH(CH₃)—CH₂—CH₃, —B—CH₂—CH(CH₃)—CH₃, wherein B is an aromatic moiety, e.g., phenyl. Alkylaryl is optionally substituted, i.e., the aryl group and/or the alkyl group, can be substituted as disclosed herein. Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g., C₆H₅—O—, for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy, and 6 to 10 ring carbon atoms, i.e., C₆₋₁₀ aryloxy. Examples of aryloxy moieties include, but are not limited to phenoxy, naphthoxy, and anthroxy.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “sugar” or “sugar group” or “sugar residue” refers to a carbohydrate moiety which may comprise 3-carbon (those) units, 4-carbon (tetrose) units, 5-carbon (pentose) units, 6-carbon (hexose) units, 7-carbon (heptose) units, or combinations thereof, and may be a monosaccharide, a disaccharide, a trisaccharide, a tetrasaccharide, a pentasaccharide, an oligosaccharide, or any other polysaccharide. In some instances, a “sugar” or “sugar group” or “sugar residue” comprises furanoses (e.g., ribofuranose, fructofuranose) or pyranoses (e.g., glucopyranose, galactopyranose), or a combination thereof. In some instances, a “sugar” or “sugar group” or “sugar residue” comprises aldoses or ketoses, or a combination thereof. Non-limiting examples of monosaccharides include ribose, deoxyribose, xylose, arabinose, glucose, mannose, galactose, and fructose. Non-limiting examples of disaccharides include sucrose, maltose, lactose, lactulose, and trehalose. Other “sugars” or “sugar groups” or “sugar residues” include polysaccharides and/or oligosaccharides, including, and not limited to, amylose, amylopectin, glycogen, inulin, and cellulose. In some instances, a “sugar” or “sugar group” or “sugar residue” is an amino-sugar. In some instances, a “sugar” or “sugar group” or “sugar residue” is a glucamine residue (1-amino-1-deoxy-D-glucitol) linked to the rest of molecule via its amino group to form an amide linkage with the rest of the molecule (i.e., a glucamide).

As used herein, “inorganic acid residue” refers to the ortho- and pyrophosphoric acid, phosphoric acid, and sulphuric acid residue.

As used herein, “organic acid residue” refers to the residue of alkanecarboxylic acid, amino acid, or oligopeptide. In one embodiment, the alkanecarboxylic acid is methanoic acid; ethanoic acid; propanoic acid; butanoic acid; pentanoic acid; hexanoic acid; heptanoic acid; octanoic acid; nonanoic acid; decanoic acid; undecanoic acid; dodecanoic acid; tridecanoic acid; tetradecanoic acid; pentadecanoic acid; hexadecanoic acid; heptadecanoic acid; octadecanoic acid; nonadecanoic acid; or icosanoic acid. In one embodiment, the alkanecarboxylic acid is methanoic acid; ethanoic acid; propanoic acid; or butanoic acid

Certain groups, moieties, substituents, and atoms are depicted with a wiggly line that intersects a bond or bonds to indicate the atom through which the groups, moieties, substituents, atoms are bonded. For example, a phenyl group that is substituted with a propyl group depicted as:

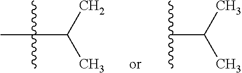

has the following structure:

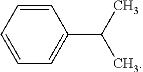

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains.

As used herein, “binding agent” refers to any molecule, e.g., antibody, capable of binding with specificity to a given binding partner, e.g., antigen.

As used herein, the term “amino acid” refers to an organic compound that contains amine (—NH₂) and carboxyl (—COOH) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, beta-alanine, ornithine and citrulline.

As used herein “peptide”, in its various grammatical forms, is defined in its broadest sense to refer to a compound of two or more subunit amino acids, amino acid analogs, or other peptidomimetics. The subunits may be linked by peptide bonds or by other bonds, for example, ester, ether, and the like. As used herein, the term “amino acid” refers to either natural and/or unnatural, proteinogenic or non-proteinogenic, or synthetic amino acids, including Glycine and both the D or L optical isomers, and amino acid analogs and peptidomimetics. If the peptide chain is short, e.g., two, three or more amino acids, it is commonly called an oligopeptide. If the peptide chain is longer, the peptide is typically called a polypeptide or a protein. Full-length proteins, analogs, mutants, and fragments thereof are encompassed by the definition. The terms also include postexpression modifications of the polypeptide, for example, glycosylation, acetylation, phosphorylation and the like. Furthermore, as ionizable amino and carboxyl groups are present in the molecule, a particular peptide may be obtained as an acidic or basic salt, or in neutral form. A peptide may be obtained directly from the source organism, or may be recombinantly or synthetically produced.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

As used herein, the term “anti-HER2 antibody” refers to an antibody selectively binding to the HER2 receptor, e.g., trastuzumab. In one embodiment, trastuzumab can be made and used as described in U.S. Pat. Nos. 6,407,213, and 5,821,337, the entire disclosure of which is incorporated herein by reference.

As used herein, the term “anti-HER3 antibody” refers to an antibody selectively binding to the HER3 receptor, e.g., patritumab. In one embodiment, patritumab can be made and used as described in U.S. Pat. No. 7,705,130, the entire disclosure of which is incorporated herein by reference.

As used herein, the term “anti-PTK7 antibody” refers to an antibody selectively binding to the PTK7 receptor, e.g., cofetuzumab. In one embodiment, cofetuzumab can be made and used as described in U.S. Pat. No. 9,777,070, the entire disclosure of which is incorporated herein by reference.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

As used herein, the term “cell-killing activity” refers to the activity that decreases or reduces the cell viability of the tested cell line.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described.

### 7.2. Conjugates

In some other examples, set forth herein is an ADC, or a pharmaceutically acceptable salt thereof, comprising: a protein linked to at least one payload moiety and linked to at least one hydrophilic moiety via a covalent linker, wherein said covalent linker is bonded directly or indirectly to each of the protein, the payload moiety, and the hydrophilic moiety. In some embodiments, the protein is an antibody or antigen binding fragment thereof.

As illustrated herein, in some examples, the binding agent is bonded directly to a covalent linker, such as a linker set forth herein. This means that the binding agent is one bond position away from the covalent linker set forth herein. In some of these examples, the covalent linker is also bonded directly to a payload moiety. This means that the covalent linker is one bond position away from a payload such as, but not limited to, Dxd, MMAE, or a stereoisomer thereof, or any payload set forth herein. In some of these examples, the covalent linker is also bonded directly to a hydrophilic moiety. This means that the covalent linker is one bond position away from a hydrophilic residue, such as the hydrophilic residues set forth herein. In some of these examples, the covalent linker is a covalent linker set forth herein.

In other examples, the binding agent is bonded indirectly to a covalent linker. This means that the binding agent is more than one bond position away from the covalent linker.

This also means that the binding agent is bonded through another moiety to the covalent linker. For example, the binding agent may be bonded to a maleimide group which is bonded to a polyethylene glycol group which is bonded to the covalent linker. In some of these examples, the covalent linker is also bonded indirectly to a payload moiety. This means that the covalent linker is more than one bond position away from a payload such as, but not limited to, Dxd, MMAE, or a stereoisomer thereof, or any payload set forth herein. This also means that the covalent linker is bonded through another moiety to the payload. For example, the covalent linker may be bonded to a dipeptide, such as but not limited to Val-Ala or Val-Cit, which may be bonded to PAB which may be bonded to the payload. In some of these examples, the covalent linker is also bonded indirectly to a hydrophilic moiety. This means that the covalent linker is more than one bond position away from a hydrophilic moiety, such as the hydrophilic residues set forth herein. This also means that the covalent linker is bonded through another moiety to the hydrophilic moiety.

In certain instances, the hydrophilic residue comprises a terminal hydrophilic group. In some instances, the hydrophilic residue comprises at least one sugar residue. In some instances, the hydrophilic residue comprises a sugar residue. In some cases, the hydrophilic residue comprises a terminal sugar residue. In further instances, the hydrophilic residue comprises more than one sugar residue. In some cases, the hydrophilic residue comprises more than one terminal sugar residue.

In another embodiment, the payload provided here is a chromophore functional group, for which the compound provided herein can be used for detection, monitoring, or study the interaction of the cell binding molecule with a target cell. Chromophore functional groups are functional groups that have the ability to absorb a kind of light, such as UV light, florescent light, IR light, near IR light, visual light. A chromatophore functional group is a functional group selected from a class or subclass of xanthophores, erythrophores, iridophores, leucophores, melanophores, and cyanophores; a class or subclass of fluorophore molecules which are fluorescent chemical compounds re-emitting light upon light; a class or subclass of visual phototransduction molecules; a class or subclass of photophore molecules; a class or subclass of luminescence molecules; and a class or subclass of luciferin compounds.

### 7.2.1. Aspect 1

Described herein are compounds according to Formula (I):

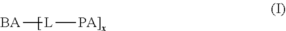

or a pharmaceutically acceptable salt tautomer, solvate, stereoisomer, enantiomer, isotopologue, or prodrug thereof,
wherein BA is a binding agent selected from a humanized, chimeric, or human antibody or an antigen binding antibody fragment of an antibody; L is a covalent linker; PA is a payload residue; and subscript x is from 1 to 30. In some instances, x is from 1 to 4. In some instances, x is about 1. In some instances, x is about 2. In some instances, x is about 3. In some instances, x is about 4.

In one example, BA is an antibody. In one example, the antibody is a humanized, chimeric, or human antibody or an antigen binding antibody fragment of an antibody. In one example, the antibody is a humanized, chimeric, or human anti-HER2 or anti-HER3 antibody or an antigen binding antibody fragment of an anti-HER2 or anti-HER3 antibody. In one example, the antibody is a monoclonal antibody.

In one example, BA is an antibody. In one example, the antibody is a humanized, chimeric, or human antibody or an antigen binding antibody fragment of cofetuzumab, patritumab, or trastuzumab.

In certain embodiments, the antibody as described herein binds to one or more of receptors selected from the group consisting of: CD7, CD19, CD22, CD27, CD30, CD33, CD37, CD70, CD74, CD79b, CD138, CD142, CA6, p-Cadherin, CEA, CEACAM5, C4.4a, DLL3, EGFR, EGFRVIII, ENPP3, EphA2, EphrinA, FLOR1, FGFR2, GCC, HER2, HER3, cKIT, LIV1, LY6E, MSLN, MUC16, NaPi2b, Nectin4, gpNMB, PSMA, SLITRK6, STEAP1, TROP2, 5T4, SSEA4, GloboH, Gb5, STn, and Tn.

In certain embodiments, the antibody as described herein binds to one or more of receptors selected from the group consisting of: B7H3, MUC1, FGFR2b, CLL1, CCR7, GPC1, and GPC3.

In certain embodiments, the antibody as described herein binds to CEA receptors.

In certain embodiments, the antibody as described herein is a bispecific antibody.

### 7.2.2. Aspect 2

Described herein are compounds according to Formula (Ia):

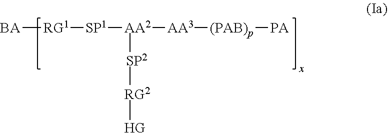

or a pharmaceutically acceptable salt tautomer, solvate, stereoisomer, enantiomer, isotopologue, or prodrug thereof,
wherein RG¹ is a reactive group residue; RG² is an optional reactive group residue; SP¹ and SP² are independently, in each instance, an optional spacer group residue; HG is a hydrophilic residue; PAB is an optional self-immolative unit; subscript p is 0 or 1; and subscript x is from 1 to 30.

In some embodiments, x is from 1 to 15. In some embodiments, x is from 2 to 10. In some embodiments, x is from 3 to 9. In one embodiment, x is about 3. In one embodiment, x is about 4. In one embodiment, x is about 5. In one embodiment, x is about 6. In one embodiment, x is about 7. In one embodiment, x is about 8. In one embodiment, x is about 9.

In some embodiments, a compound of Formula (Ia) is a compound having P3 modification, wherein AA² comprises formula (W):

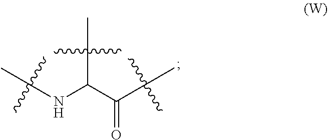 [SMILES: *NC(*)C(*)=O]

and
AA³ is a dipeptide residue of -valine-alanine-, -valine-citrulline-, or

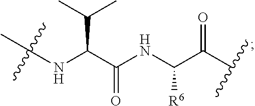

wherein R⁶ is —CH₃, or —(CH₂)₃—NHC(═O)NH₂.

In one embodiment,

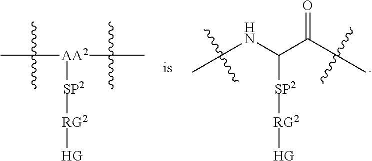

In some embodiments, PAB represents —NH—CH2-O—, formula (Y1):

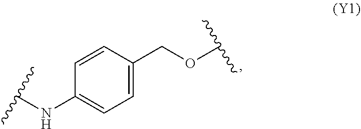 [SMILES: *Nc1ccc(CO*)cc1]

or
formula (Y2):

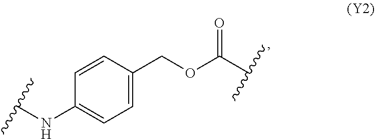 [SMILES: *Nc1ccc(COC(*)=O)cc1]

wherein the

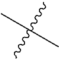

indicates the bond through which the PAB is bonded to the adjacent groups in the formula.

In some embodiments, RG¹ is

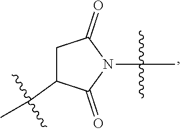 [SMILES: *C1CC(=O)N(*)C1=O]

### -(Succinimid-3-yl-N),

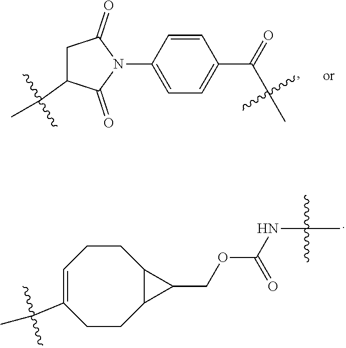 [SMILES: *C(=O)c1ccc(N2C(=O)CC(*)C2=O)cc1 ; *NC(=O)OCC1C2CC/C=C(/*)CCC21]

In some embodiments, RG¹ is

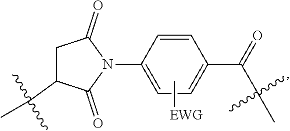

wherein EWG is an electrowithdrawn group, e.g., —CN, —NO₂, halogen, —CF₃, —C(═O)OR¹, and —C(═O)R¹, and R¹ is substituted or unsubstituted alky, substituted or unsubstituted cycloalkyl, substituted or unsubstituted aryl, substituted or unsubstituted heterocycloalkyl, or substituted or unsubstituted heteroaryl.

In some embodiments, RG¹ is

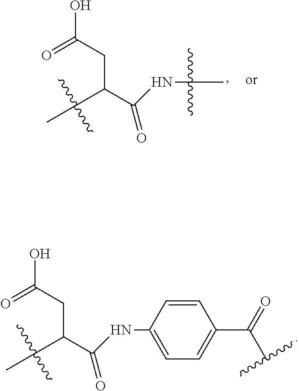 [SMILES: *NC(=O)C(*)CC(=O)O ; *C(=O)c1ccc(NC(=O)C(*)CC(=O)O)cc1]

In some embodiments, RG¹ is

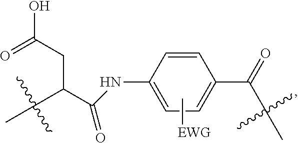

wherein EWG is an electrowithdrawn group, e.g., —CN, —NO₂, halogen, —CF₃, —C(═O)OR¹, and —C(═O)R¹, and R¹ is substituted or unsubstituted alky, substituted or unsubstituted cycloalkyl, substituted or unsubstituted aryl, substituted or unsubstituted heterocycloalkyl, or substituted or unsubstituted heteroaryl.

In some embodiments, a compound of Formula (Ia) having its ring opened is a compound of Formula (Ia), wherein RG¹ is an opened heterocyclic ring. In some embodiments, RG¹ is

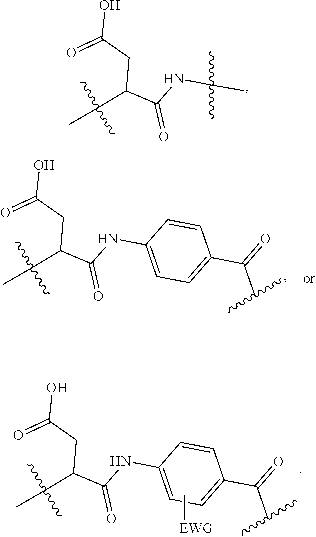

In some embodiments, RG² is a bond, —C(═O)—NH—, or —NHC(═O)—.

In some embodiments, SP¹ is —(CH₂)ₙ₁—C(═O)—, —(CH₂CH₂O)ₙ₂—CH₂CH₂—C(═O)—, —CH[—(CH₂)ₙ₃—COOH]—C(═O)—, —CH₂—C(═O)—NH—(CH₂)ₙ₄—C(═O)—, —CH₂—C(═O)—NH—(CH₂)ₙ₃—C(═O)—NH—(CH₂)ₙ₄—C(═O)—, or —C(═O) (CH₂)ₙ₅—C(═O)—, wherein each of n1, n2, n3, n4, and n5 independently represents an integer of 1 to 8.

In some embodiments, SP² is —(CH₂)ₙ₆—; and n6 represents an integer of 1 to 8.

In some embodiments, HG is

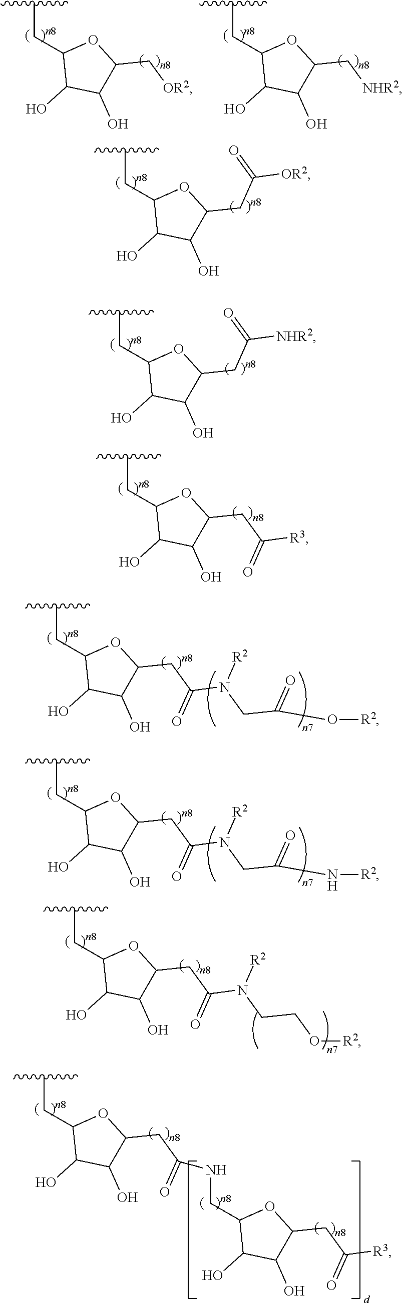

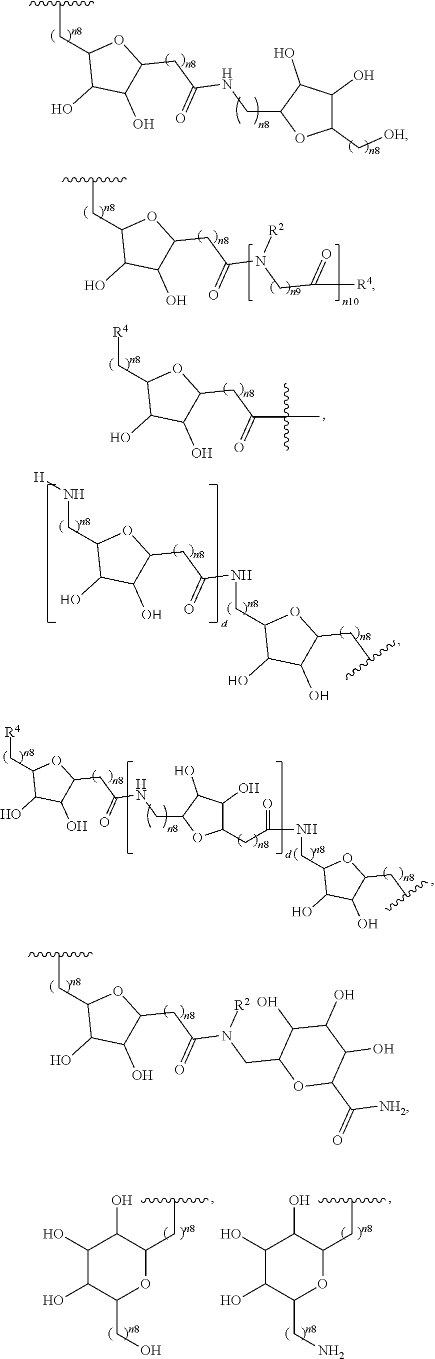

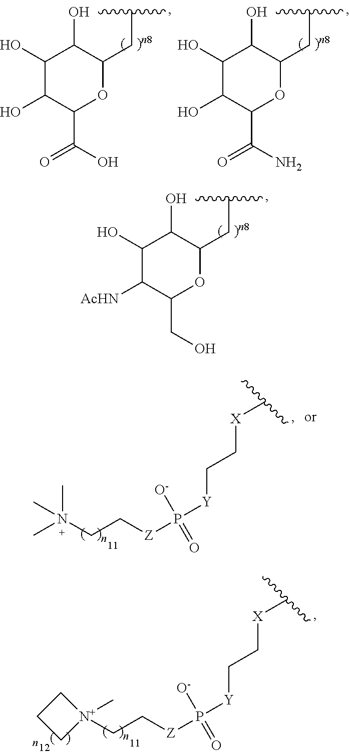

wherein each n7 is independently 1-15; each n8 is independently 0 or 1; each n9 is independently 1 or 2; each n10 is independently an integer of 4 to 16, such as 4, 8, or 12; each n11 is independently an integer of 0 to 5; n12 is an integer of 0 to 3; d is 0-3; R² is H or Me; R³ is —OH, —NH₂, —NHCH₂—CH₂—(PEG)ₓ-OH, or —NHCH₂—CH₂—(PEG)ₓ-OMe; R⁴ is OH or NH₂; and each of X, Y, and Z is independently —CH₂—, —NH—, —S— or —O—.

In some embodiments, HG is

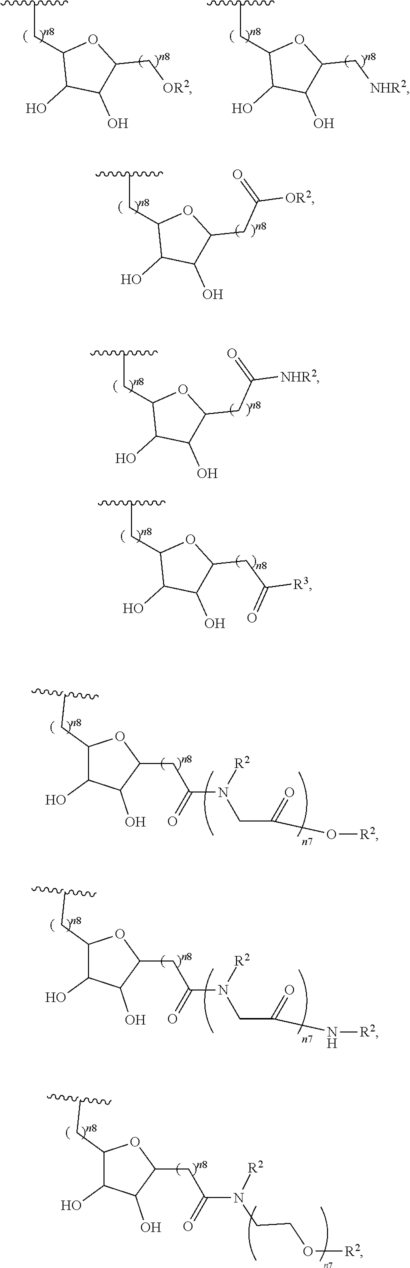

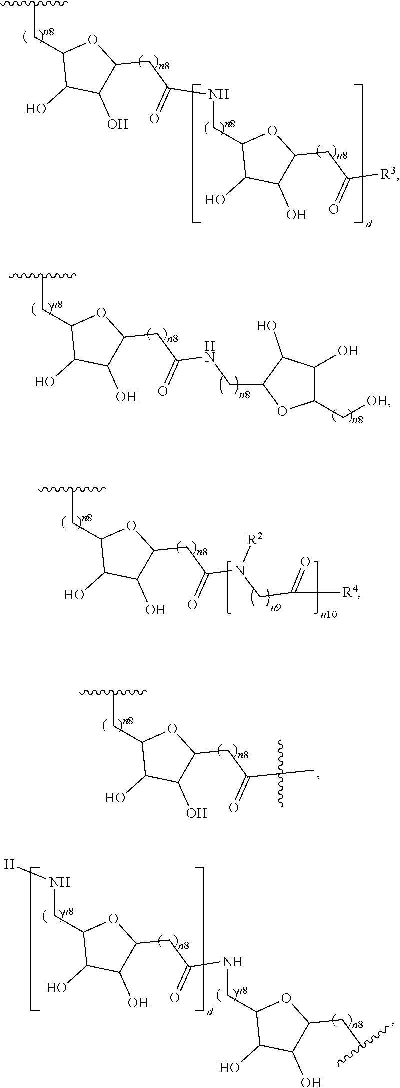

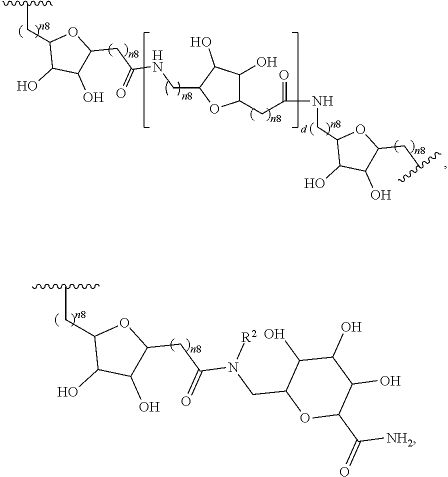

wherein each n7 is independently 1-15; each n8 is independently 0 or 1; each n9 is independently 1 or 2; each n10 is independently an integer of 4 to 16, such as 4, 8, or 12; d is 0-3; R² is H or Me; R³ is —OH, —NH₂, —NHCH₂—CH₂—(PEG)ₓ-OH, or —NHCH₂—CH₂—(PEG)ₓ-OMe; R⁴ is OH or NH₂.

In some embodiments, HG is

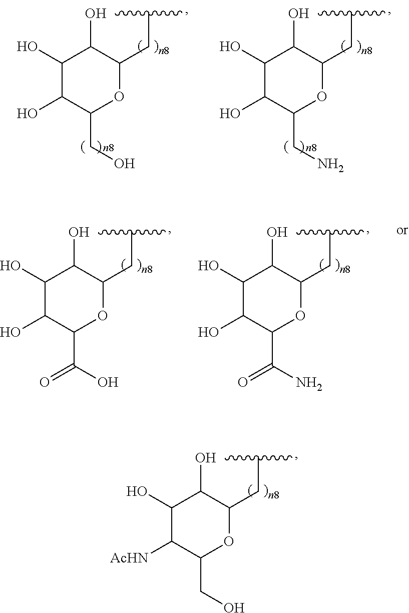

wherein each n8 is independently 0 or 1; and R¹ is H or Me.

In some embodiments, HG is

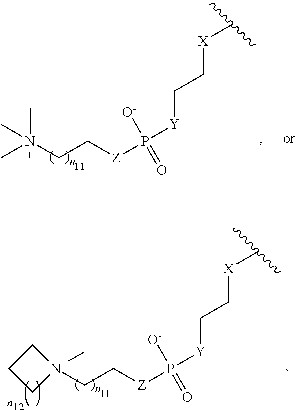

wherein each n11 is independently an integer of 0 to 5; n12 is an integer of 0 to 3; and each of X, Y, and Z is independently —CH₂—, —NH—, —S— or —O—.

In some embodiments, HG is —NHSO₂N—₂, —SO₃H, —SO₂NH₂, —PO₃H₂, and RG² is a bond.

In some embodiments, each PA independently represents a chromophore functional group.

In some embodiments, each chromophore functional group is independently a functional group selected from a class or subclass of xanthophores, erythrophores, iridophores, leucophores, melanophores, and cyanophores; a class or subclass of fluorophore molecules which are fluorescent chemical compounds re-emitting light upon light; a class or subclass of visual phototransduction molecules; a class or subclass of photophore molecules; a class or subclass of luminescence molecules; and a class or subclass of luciferin compounds.

In some embodiments, each PA is independently selected from the group consisting of Monomethyl auristatin E (MMAE), Monomethyl auristatin F (MMAF), Monomethyl auristatin D (MMAD), Mertansine (Maytansinoid DM1/DM4), Paclitaxel, Docetaxel, Epothilone B, Epothilone A, CYT997, Auristatin tyramine phosphate, Auristatin aminoquinoline, Halocombstatins, Calicheamicin theta, 7-Ethyl-10-hydroxy-camptothecin (SN-38), Pyrrolobenzodiazepine (PBD), Pancratistatin, Cyclophosphate, Cribrostatin-6, Kitastatin, Turbostatin 1-4, Halocombstatins, Eribulin, Hemiasterlin, PNU and Silstatins.

In some embodiments, each PA independently represents formula (D1):

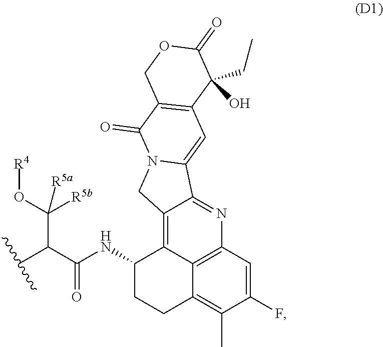

wherein each of R⁴, R^{5a}, and R^{5b} is independently hydrogen, sugar residue, substituted or unsubstituted inorganic or organic acid residue, substituted or unsubstituted C₁₋₈ alkyl, substituted or unsubstituted cycloalkyl, substituted or unsubstituted non-aromatic heterocyclyl, substituted or unsubstituted cycloalkylalkyl, or substituted or unsubstituted heterocyclylalkyl;
R^{5a} and R^{5b} together with the atoms to which they are attached, form a substituted or unsubstituted cycloalkyl, substituted or unsubstituted non-aromatic heterocyclyl.

In some embodiments, R⁴ is hydrogen,

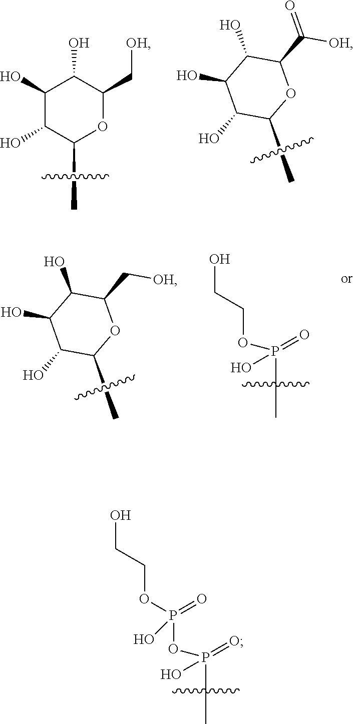

and
wherein each of R^{5a} and R^{5b} is independently H, CH₃, or CF₃; or
R^{5a} and R^{5b} together with the atoms to which they are attached, form a substituted or unsubstituted cycloalkyl, substituted or unsubstituted non-aromatic heterocyclyl.

In some embodiments, R⁴ is hydrogen,

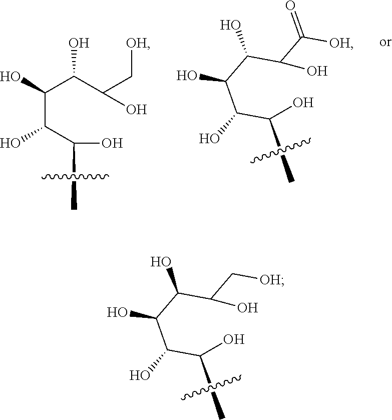

and
wherein each of R^{5a} and R^{5b} is independently H, CH₃, or CF₃; or
R^{5a} and R^{5b} together with the atoms to which they are attached, form a substituted or unsubstituted cycloalkyl, substituted or unsubstituted non-aromatic heterocyclyl.

In some embodiments, each PA independently represents

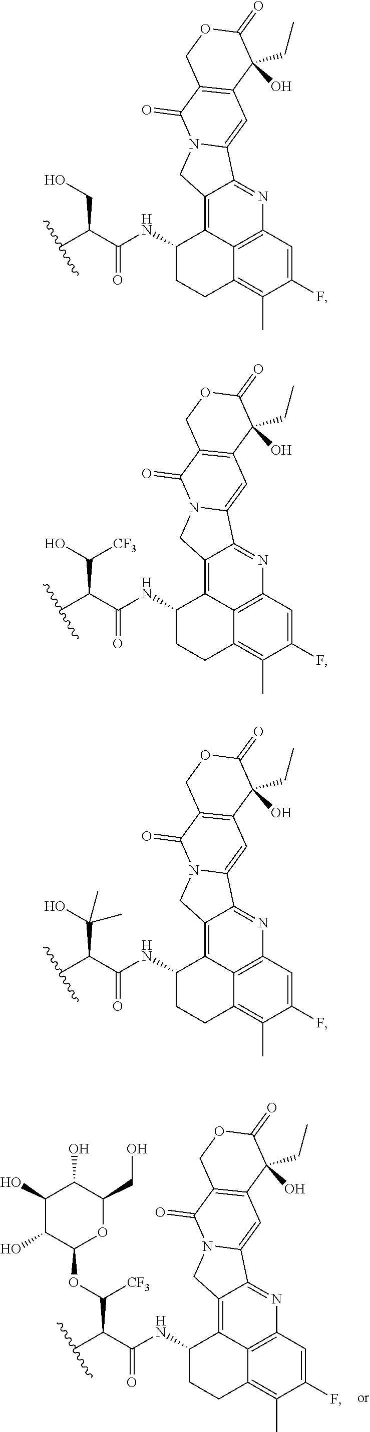

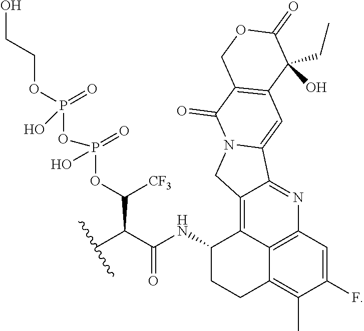 [SMILES: *[C@H](C(=O)N[C@H]1CCc2c(C)c(F)cc3nc4c(c1c23)Cn1c-4cc2c(c1=O)COC(=O)[C@]2(O)CC)C(OP(=O)(O)OP(=O)(O)OCCO)C(F)(F)F]

In some embodiments, each PA independently represents formula (D2):

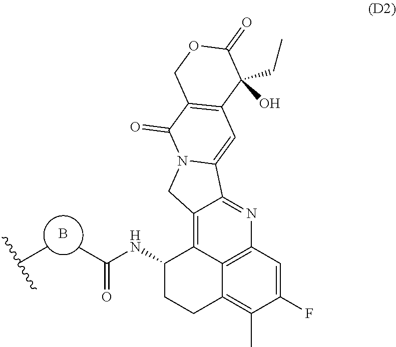

wherein ring B is a substituted or unsubstituted cycloalkyl, substituted or unsubstituted aryl, substituted or unsubstituted heterocyclyl, or substituted or unsubstituted heteroaryl.

In some embodiments, each PA independently represents

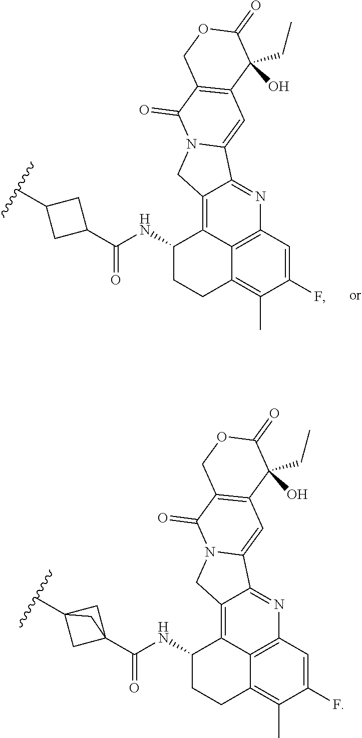 [SMILES: *C1CC(C(=O)N[C@H]2CCc3c(C)c(F)cc4nc5c(c2c34)Cn2c-5cc3c(c2=O)COC(=O)[C@]3(O)CC)C1 ; "or" ; *C12CC(C(=O)N[C@H]3CCc4c(C)c(F)cc5nc6c(c3c45)Cn3c-6cc4c(c3=O)COC(=O)[C@]4(O)CC)(C1)C2]

In some embodiments, each PA independently represents formula (D33):

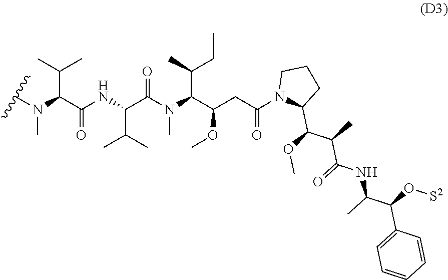

wherein S² is an enzyme hydrolyzable hydrophilic group.

In some embodiments, the S² group is hydrogen or represents one of the following formulas:

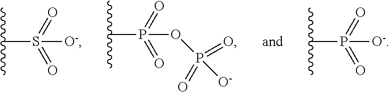

In some embodiments, each PA independently represents formula (E1):

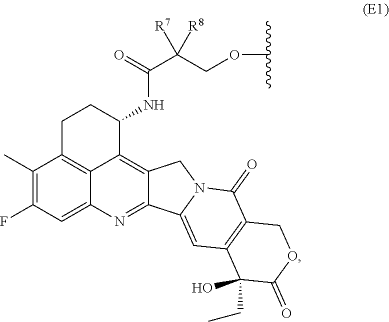

wherein
each of R⁷ and R⁸ is, independently, hydrogen, halogen, or alkyl.

In one embodiment, R⁷ and R⁸ are hydrogen.

In one embodiment, R⁷ and R⁸ are methyl.

In one embodiment, R⁷ is methyl and R⁸ is F.

In one embodiment, the carbon to which R⁷ and R⁸ connect is in the S configuration.

In one embodiment, the carbon to which R⁷ and R⁸ connect is in the R configuration.

In some embodiments, each PA independently represents the following formula:

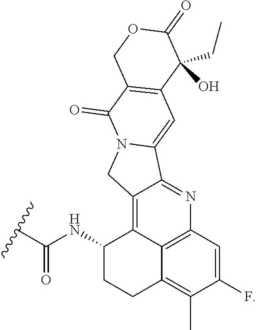 [SMILES: *C(=O)N[C@H]1CCc2c(C)c(F)cc3nc4c(c1c23)Cn1c-4cc2c(c1=O)COC(=O)[C@]2(O)CC]

In some embodiments, each PA is independently Dxd, or independently represents the following formula:

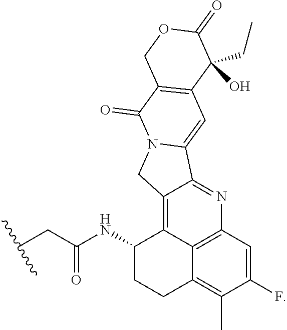 [SMILES: *CC(=O)N[C@H]1CCc2c(C)c(F)cc3nc4c(c1c23)Cn1c-4cc2c(c1=O)COC(=O)[C@]2(O)CC]

In some embodiments, each PA independently represents the following formula:

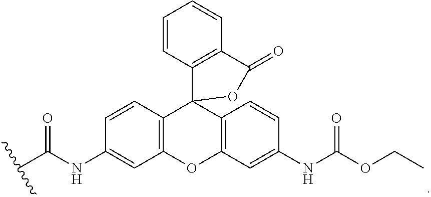 [SMILES: *C(=O)Nc1ccc2c(c1)Oc1cc(NC(=O)OCC)ccc1C21OC(=O)c2ccccc21]

In some embodiments,

AA² is an amino acid residue of glycine or

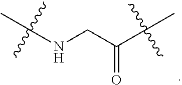 [SMILES: *NCC(*)=O]

### 7.2.3. Aspect 3

Described herein are compounds of Formula (Ia) or a pharmaceutically acceptable salt tautomer, solvate, stereoisomer, enantiomer, isotopologue, or prodrug thereof, wherein AA² comprises formula (W):

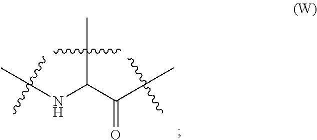 [SMILES: *NC(*)C(*)=O]

and
AA³ is a tetrapeptide residue of -glycine-glycine-phenylalanine-glycine- or

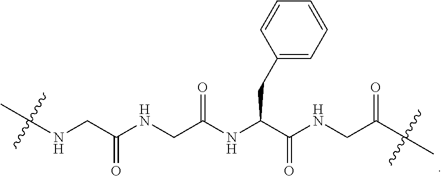 [SMILES: *NCC(=O)NCC(=O)N[C@@H](Cc1ccccc1)C(=O)NCC(*)=O]

In some embodiments, x is from 1 to 15. In some embodiments, x is from 2 to 10. In some embodiments, x is from 3 to 9. In one embodiment, x is about 3. In one embodiment, x is about 4. In one embodiment, x is about 5. In one embodiment, x is about 6. In one embodiment, x is about 7. In one embodiment, x is about 8. In one embodiment, x is about 9.

In one embodiment, the BA, RG¹, SP¹, SP², RG², HG, PAB, p, and PA are as provided herein.

### 7.2.4. Aspect 4

Described herein are compounds according to Formula (Ib):

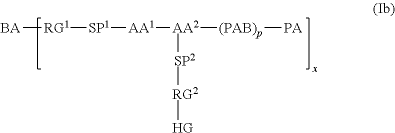

or a pharmaceutically acceptable salt tautomer, solvate, stereoisomer, enantiomer, isotopologue, or prodrug thereof,
wherein AA² comprises formula (W):

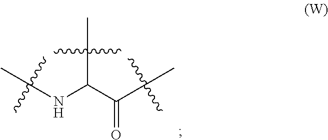 [SMILES: *NC(*)C(*)=O]

and
AA¹ is a dipeptide residue of -valine-alanine-, -valine-citrulline-, or

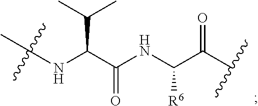

wherein R⁶ is —CH₃, or —(CH₂)₃—NHC(═O)NH₂.

In one embodiment,

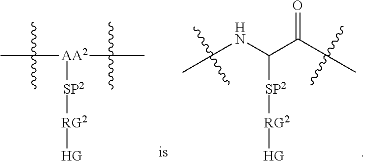

In some embodiments, x is from 1 to 15. In some embodiments, x is from 2 to 10. In some embodiments, x is from 3 to 9. In one embodiment, x is about 3. In one embodiment, x is about 4. In one embodiment, x is about 5. In one embodiment, x is about 6. In one embodiment, x is about 7. In one embodiment, x is about 8. In one embodiment, x is about 9.

In some embodiments, a compound of Formula (Ib) having its ring opened is a compound of Formula (Ib), wherein RG¹ is an opened heterocyclic ring. In some embodiments, a compound of Formula (Ib) with ring opening is a compound of Formula (Ib), wherein RG¹ is an opened heterocyclic ring. In some embodiments, RG¹ is

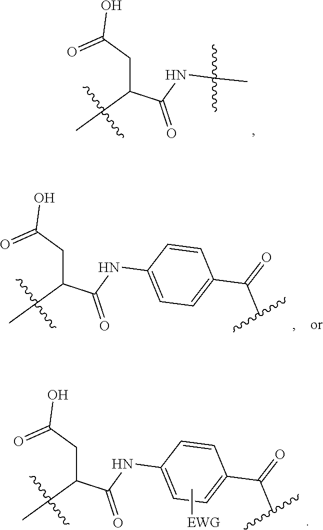

In one embodiment, the BA, RG¹, SP¹, SP², RG², HG, PAB, p, and PA are as provided herein.

### 7.2.5. Aspect 5

Described herein are compounds of Formula (Ib) or a pharmaceutically acceptable salt tautomer, solvate, stereoisomer, enantiomer, isotopologue, or prodrug thereof, wherein AA² comprises formula (W):

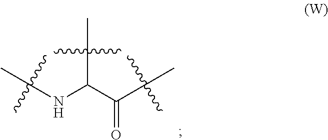 [SMILES: *NC(*)C(*)=O]

and
AA¹ is a tetrapeptide residue of -glycine-glycine-phenylalanine-glycine- or

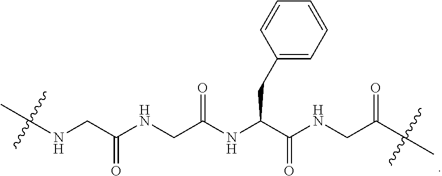 [SMILES: *NCC(=O)NCC(=O)N[C@@H](Cc1ccccc1)C(=O)NCC(*)=O]

In one embodiment, the BA, RG¹, SP¹, SP², RG², HG, PAB, p, x, and PA are as provided herein.

### 7.2.6. Aspect 6

Described herein are compounds according to Formula (Ic):

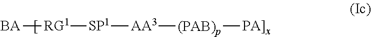

or a pharmaceutically acceptable salt tautomer, solvate, stereoisomer, enantiomer, isotopologue, or prodrug thereof,
wherein AA³ is a dipeptide residue of -valine-alanine-, -valine-citrulline-, or

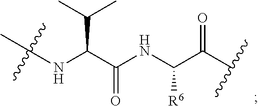

wherein R⁶ is —CH₃, or —(CH₂)₃—NHC(═O)NH₂.

In some embodiments, x is from 1 to 15. In some embodiments, x is from 2 to 10. In some embodiments, x is from 3 to 9. In one embodiment, x is about 3. In one embodiment, x is about 4. In one embodiment, x is about 5. In one embodiment, x is about 6. In one embodiment, x is about 7. In one embodiment, x is about 8. In one embodiment, x is about 9.

In some embodiments, a compound of Formula (Ic) having its ring opened is a compound of Formula (Ic), wherein RG¹ is an opened heterocyclic ring. In some embodiments, a compound of Formula (Ic) with ring opening is a compound of Formula (Ic), wherein RG¹ is an opened heterocyclic ring. In some embodiments, RG¹ is

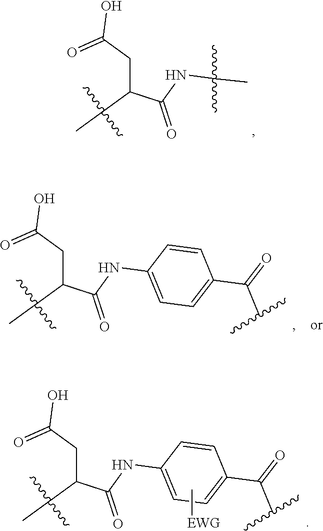

In one embodiment, the BA, RG¹, SP¹, PAB, p, x, and PA are as provided herein.

### 7.2.7. Aspect 7

Described herein are compounds according to Formula (Ic) or pharmaceutically acceptable salts, solvates, stereoisomers, or derivatives thereof, wherein AA³ is a tetrapeptide residue of -glycine-glycine-phenylalanine-glycine- or

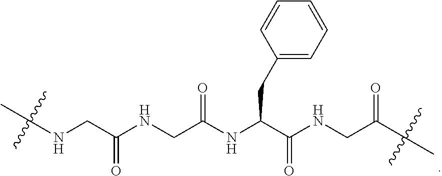 [SMILES: *NCC(=O)NCC(=O)N[C@@H](Cc1ccccc1)C(=O)NCC(*)=O]

In one embodiment, the BA, RG¹, SP¹, PAB, p, x, and PA are as provided herein.

### 7.2.8. Aspect 8

In some embodiments, the compound is selected from the group consisting of the compounds in Table 3. In some embodiments, the compound is selected from the group consisting of ADC2-62-1, ADC2-62-2, ADC2-63-1, ADC2-63-2, ADC2-64-1, ADC2-64-2, ADC2-65-1, and ADC2-65-2.

### 7.3. Linkers with Payloads (Platform)

In some embodiments, set forth herein is a compound comprises a reactive linker bonded to at least one payload moiety.

In some embodiments, set forth herein is a compound, or a pharmaceutically acceptable solvate, stereoisomer, or derivative thereof, comprising a payload unit linked to at least one hydrophilic residue via a covalent linker unit, wherein the covalent linker is bonded directly or indirectly to each of the payload unit, and the hydrophilic residue.

In one embodiment, the hydrophilic residue comprises a terminal hydrophilic group.

In one embodiment, the hydrophilic residue comprises a sugar residue.

### 7.3.1. Aspect 9

In some embodiments, set forth herein is a compound having formula (II):

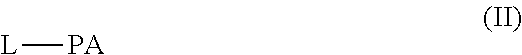

or a pharmaceutically acceptable salt tautomer, solvate, stereoisomer, enantiomer, isotopologue, or prodrug thereof, wherein L is a covalent linker; and PA is a payload residue.

### 7.3.2. Aspect 10

Described herein are compounds according to Formula (IIa):

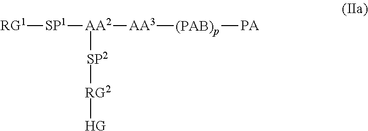

or a pharmaceutically acceptable salt tautomer, solvate, stereoisomer, enantiomer, isotopologue, or prodrug thereof,
wherein AA² comprises formula (W):

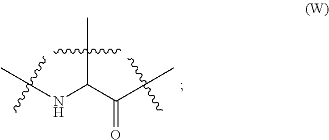 [SMILES: *NC(*)C(*)=O]

and
AA³ is a dipeptide residue of -valine-alanine-, -valine-citrulline-, or

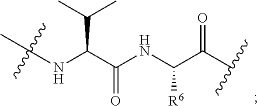

wherein R⁶ is —CH₃, or —(CH₂)₃—NHC(═O)NH₂.

In one embodiment,

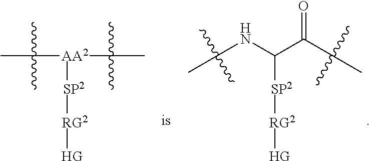

In one embodiment, the RG¹ is

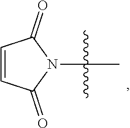 [SMILES: *N1C(=O)C=CC1=O]

### Succinimid-N—,

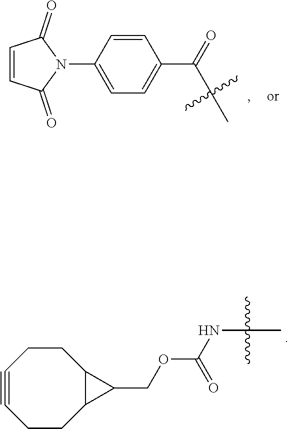 [SMILES: *C(=O)c1ccc(N2C(=O)C=CC2=O)cc1 ; *NC(=O)OCC1C2CCC#CCCC21]

In one embodiment, the RG¹ is

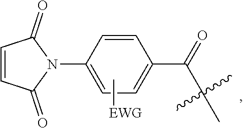

wherein EWG is an electrowithdrawn group, e.g., —CN, —NO₂, halogen, —CF₃, —C(═O)OR¹, and —C(═O)R¹, and R¹ is substituted or unsubstituted alky, substituted or unsubstituted cycloalkyl, substituted or unsubstituted aryl, substituted or unsubstituted heterocycloalkyl, or substituted or unsubstituted heteroaryl.

In one embodiment, the RG¹ is

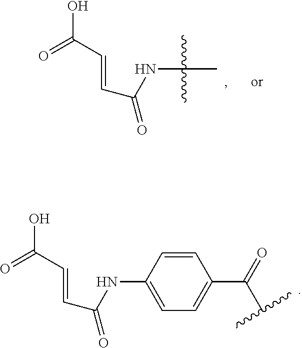 [SMILES: *NC(=O)/C=C/C(=O)O ; *C(=O)c1ccc(NC(=O)/C=C/C(=O)O)cc1]

In one embodiment, the RG¹ is

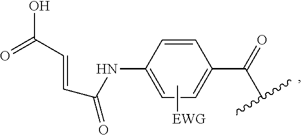

wherein EWG is an electrowithdrawn group, e.g., —CN, —NO₂, halogen, —CF₃, —C(═O)OR¹, and —C(═O)R¹, and R¹ is substituted or unsubstituted alky, substituted or unsubstituted cycloalkyl, substituted or unsubstituted aryl, substituted or unsubstituted heterocycloalkyl, or substituted or unsubstituted heteroaryl.

In some embodiments, a compound of Formula (II) having its ring opened is a compound of Formula (II), wherein RG¹ is an opened heterocyclic ring. In some embodiments, a compound of Formula (II) with ring opening is a compound of Formula (II), wherein RG¹ is an opened heterocyclic ring. In some embodiments, RG¹ is

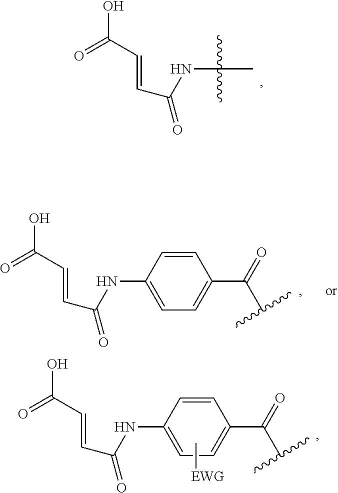

wherein EWG is an electrowithdrawn group, e.g., —CN, —NO₂, halogen, —CF₃, —C(═O)OR¹, and —C(═O)R¹, and R¹ is substituted or unsubstituted alky, substituted or unsubstituted cycloalkyl, substituted or unsubstituted aryl, substituted or unsubstituted heterocycloalkyl, or substituted or unsubstituted heteroaryl.

In some embodiments, a compound of Formula (IIa) having its ring opened is a compound of Formula (IIa), wherein RG¹ is an opened heterocyclic ring. In some embodiments, a compound of Formula (IIa) with ring opening is a compound of Formula (IIa), wherein RG¹ is an opened heterocyclic ring. In some embodiments, RG¹ is

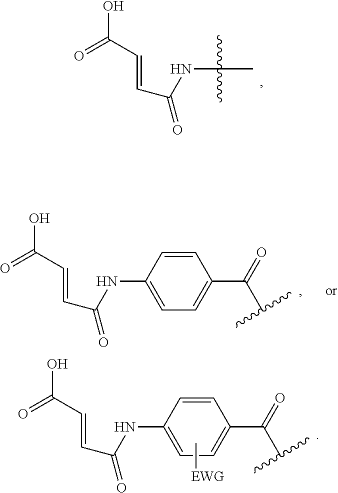

In one embodiment, the RG¹, SP¹, SP², RG², HG, PAB, p, and PA are as provided herein.

### 7.3.3. Aspect 11

Described herein are compounds according to Formula (IIa or a pharmaceutically acceptable salt tautomer, solvate, stereoisomer, enantiomer, isotopologue, or prodrug thereof, wherein AA² comprises formula (W):

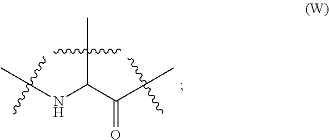 [SMILES: *NC(*)C(*)=O]

and
AA³ is a tetrapeptide residue of -glycine-glycine-phenylalanine-glycine- or

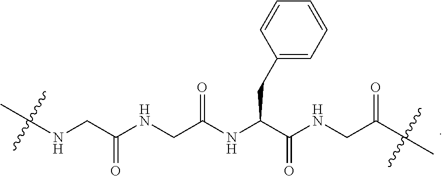 [SMILES: *NCC(=O)NCC(=O)N[C@@H](Cc1ccccc1)C(=O)NCC(*)=O]

In one embodiment, the RG¹, SP¹, SP², RG², HG, PAB, p, and PA are as provided herein.

### 7.3.4. Aspect 12

Described herein are compounds according to Formula (IIb):

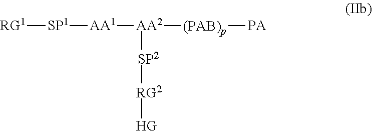

or a pharmaceutically acceptable salt tautomer, solvate, stereoisomer, enantiomer, isotopologue, or prodrug thereof,
wherein AA² comprises formula (W):

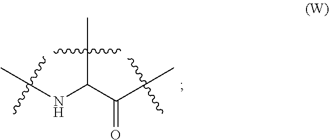 [SMILES: *NC(*)C(*)=O]

and
AA¹ is a dipeptide residue of -valine-alanine-, -valine-citrulline-, or

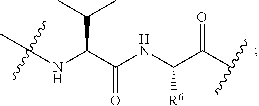

wherein R⁶ is —CH₃, or —(CH₂)₃—NHC(═O)NH₂.

In one embodiment,

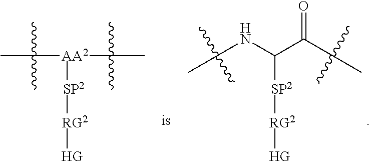

In some embodiments, a compound of Formula (IIb) having its ring opened is a compound of Formula (IIb), wherein RG¹ is an opened heterocyclic ring. In some embodiments, RG¹ is

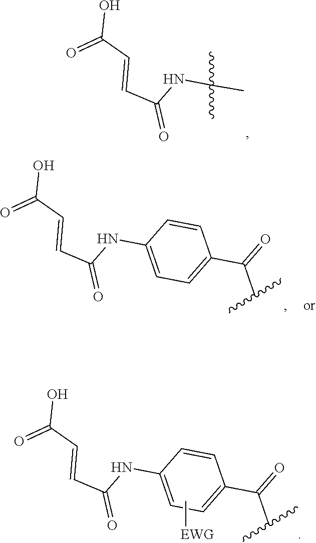

In one embodiment, the RG¹, SP¹, SP², RG², HG, PAB, p, and PA are as provided herein.

### 7.3.5. Aspect 13

Described herein are compounds according to Formula (IIb) or a pharmaceutically acceptable salt tautomer, solvate, stereoisomer, enantiomer, isotopologue, or prodrug thereof, wherein AA² comprises formula (W):

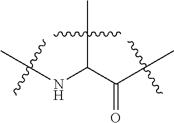 [SMILES: *NC(*)C(*)=O]

(W); and
   AA¹ is a tetrapeptide residue of -glycine-glycine-phenylalanine-glycine- or

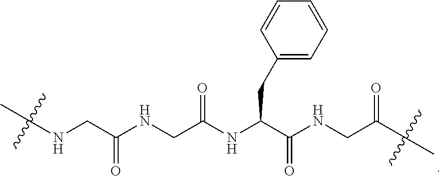 [SMILES: *NCC(=O)NCC(=O)N[C@@H](Cc1ccccc1)C(=O)NCC(*)=O]

In one embodiment, the RG¹, SP¹, SP², RG², HG, PAB, p, and PA are as provided herein.

### 7.3.6. Aspect 14

Described herein are compounds according to Formula (IIc):

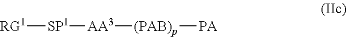

or a pharmaceutically acceptable salt tautomer, solvate, stereoisomer, enantiomer, isotopologue, or prodrug thereof,
wherein AA³ is a dipeptide residue of -valine-alanine-, -valine-citrulline-, or

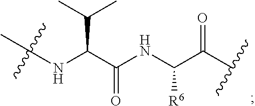

wherein R⁶ is —CH₃, or —(CH₂)₃—NHC(═O)NH₂.

In some embodiments, a compound of Formula (IIc) having its ring opened is a compound of Formula (IIc), wherein RG¹ is an opened heterocyclic ring. In some embodiments, RG¹ is

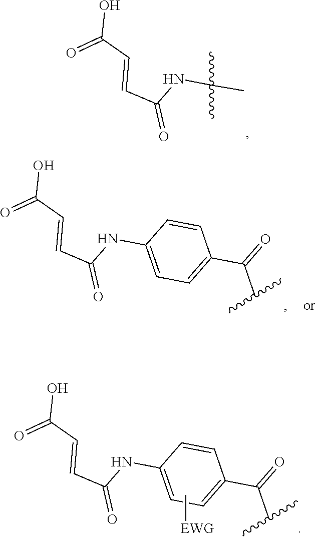

In one embodiment, the RG¹, SP¹, PAB, p, and PA are as provided herein.

### 7.3.7. Aspect 15

Described herein are compounds according to Formula (IIc) or pharmaceutically acceptable salts, solvates, stereoisomers, or derivatives thereof,

wherein: RG¹ is a reactive group residue; SP¹ is an optional spacer group residue; PAB is an optional self-immolative unit; subscript p is 0 or 1; and PA is a payload residue;
wherein AA³ is a tetrapeptide residue of -glycine-glycine-phenylalanine-glycine- or

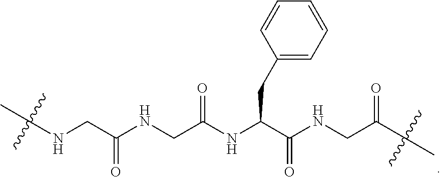 [SMILES: *NCC(=O)NCC(=O)N[C@@H](Cc1ccccc1)C(=O)NCC(*)=O]

In one embodiment, the RG¹, SP¹, PAB, p, and PA are as provided herein.

### 7.3.8. Aspect 16

In some embodiments, the compound is selected from the group consisting of the compounds in Table 1.

### 7.3.9. Aspect 17

Described herein is a ligand-drug conjugate or a pharmaceutically acceptable salt or solvate thereof, wherein the ligand-drug conjugate comprises a structure of formula (E1):

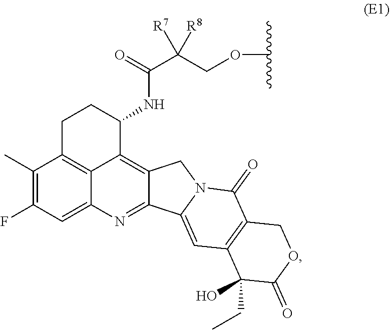

wherein
each of R⁷ and R⁸ is, independently, hydrogen, halogen, or alkyl.

In some embodiments, the ligand-drug conjugate comprises a structure of formula (E2):

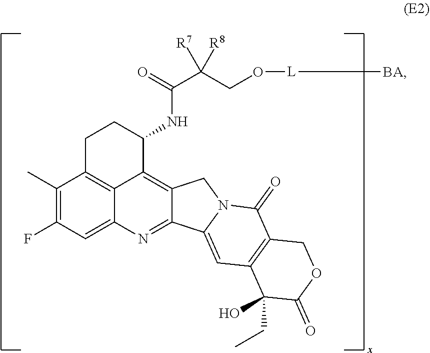

wherein
BA is a binding agent selected from a humanized, chimeric, or human antibody or an antigen binding antibody fragment of an antibody;
L is a covalent linker as described here; and
x is 1 to 10, which can be an integer or a decimal.

In some embodiments, the antibody is patritumab, cofetuzumab, or trastuzumab. In one embodiment, the antibody is patritumab.

In one embodiment, R⁷ and R⁸ are hydrogen.

In one embodiment, R⁷ and R⁸ are methyl.

In one embodiment, R⁷ is methyl and R⁸ is F.

In one embodiment, the carbon to which R⁷ and R⁸ connect is in the S configuration.

In one embodiment, the carbon to which R⁷ and R⁸ connect is in the R configuration.

In some embodiments, the ligand-drug conjugate is selected from Table 24. In one embodiment, the antibody is patritumab.

### 7.3.10. Aspect 18

Described herein is a compound having formula (E3):

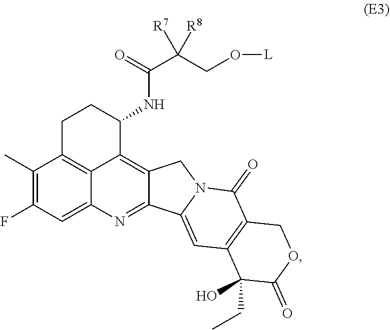

or a pharmaceutically acceptable solvate, stereoisomer, or derivative thereof, wherein
L is a covalent linker as described here;
each of R⁷ and R⁸ is, independently, hydrogen, halogen, or alkyl.

In one embodiment, R⁷ and R⁸ are hydrogen.

In one embodiment, R⁷ and R⁸ are methyl.

In one embodiment, R⁷ is methyl and R⁸ is F.

In one embodiment, the carbon to which R⁷ and R⁸ connect is in the S configuration.

In one embodiment, the carbon to which R⁷ and R⁸ connect is in the R configuration.

In one embodiment, the compound is selected from Table 25.

### 7.4. Payload

Provided herein is a compound, or pharmaceutically acceptable solvate, stereoisomer, or derivative thereof, wherein the compound is a compound having formula (D4):

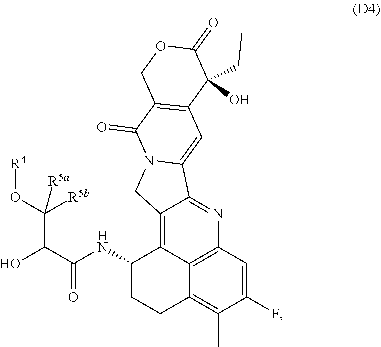

wherein each of R⁴, R^{5a}, and R^{5b} is independently hydrogen, sugar residue, substituted or unsubstituted inorganic or organic acid residue, substituted or unsubstituted C₁₋₈ alkyl, substituted or unsubstituted cycloalkyl, substituted or unsubstituted non-aromatic heterocyclyl, substituted or unsubstituted cycloalkylalkyl, or substituted or unsubstituted heterocyclylalkyl;
R^{5a} and R^{5b} together with the atoms to which they are attached, form a substituted or unsubstituted cycloalkyl, substituted or unsubstituted non-aromatic heterocyclyl.

In one embodiment, the compound has one of the following structures:

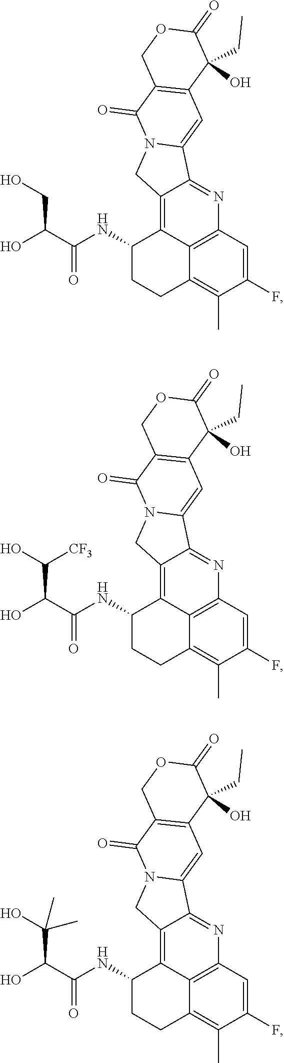

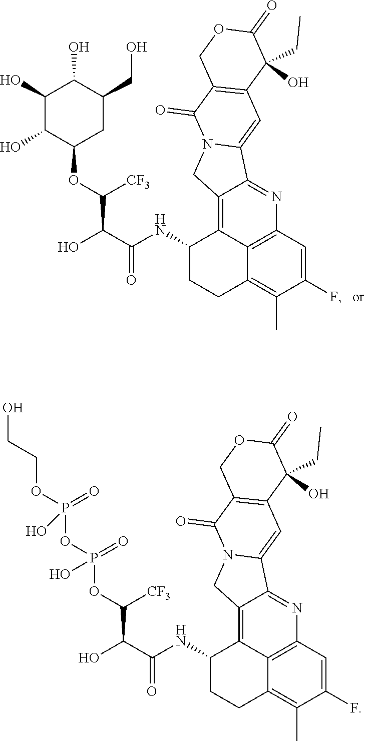 [SMILES: CC[C@@]1(O)C(=O)OCc2c1cc1n(c2=O)Cc2c-1nc1cc(F)c(C)c3c1c2[C@@H](NC(=O)[C@@H](O)C(O[C@@H]1C[C@H](CO)[C@@H](O)[C@H](O)[C@H]1O)C(F)(F)F)CC3 ; CC[C@@]1(O)C(=O)OCc2c1cc1n(c2=O)Cc2c-1nc1cc(F)c(C)c3c1c2[C@@H](NC(=O)[C@@H](O)C(OP(=O)(O)OP(=O)(O)OCCO)C(F)(F)F)CC3]

Provided herein is a compound, or pharmaceutically acceptable solvate, stereoisomer, or derivative thereof, wherein the compound is a compound having formula (D5):

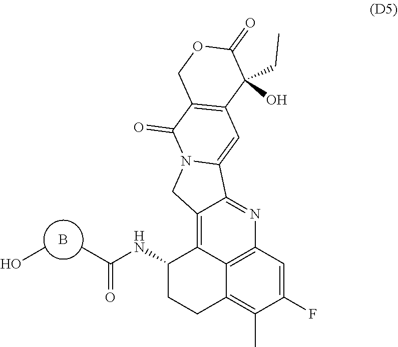

wherein ring B is a substituted or unsubstituted cycloalkyl, substituted or unsubstituted aryl, substituted or unsubstituted heterocyclyl, or substituted or unsubstituted heteroaryl.

In one embodiment, the compound has one of the following structures:

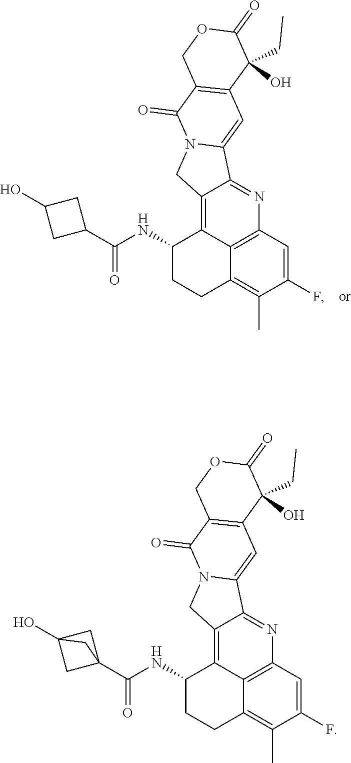 [SMILES: CC[C@@]1(O)C(=O)OCc2c1cc1n(c2=O)Cc2c-1nc1cc(F)c(C)c3c1c2[C@@H](NC(=O)C1CC(O)C1)CC3 ; "or" ; CC[C@@]1(O)C(=O)OCc2c1cc1n(c2=O)Cc2c-1nc1cc(F)c(C)c3c1c2[C@@H](NC(=O)C12CC(O)(C1)C2)CC3]

Provided herein is a compound, or a pharmaceutically acceptable solvate, stereoisomer, or derivative thereof, wherein the compound is a compound having formula (D6):

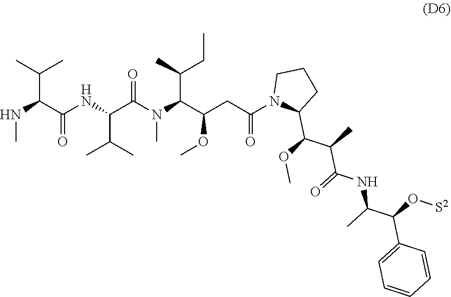

wherein S² is an enzyme hydrolyzable hydrophilic group.

In one embodiment, the S² group is as set forth herein.

Provided herein is a compound, or a pharmaceutically acceptable solvate, stereoisomer, or derivative thereof, wherein the compound is a compound formula (E4):

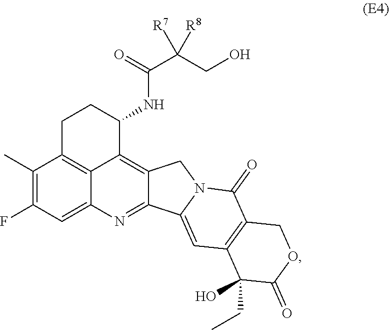

or a pharmaceutically acceptable solvate, stereoisomer, or derivative thereof, wherein
each of R⁷ and R⁸ is, independently, hydrogen, halogen, or alkyl.

In one embodiment, R⁷ and R⁸ are hydrogen.

In one embodiment, R⁷ and R⁸ are methyl.

In one embodiment, R⁷ is methyl, and R⁸ is F.

In one embodiment, the carbon to which R⁷ and R⁸ connect is in the S configuration.

In one embodiment, the carbon to which R⁷ and R⁸ connect is in the R configuration.

In one embodiment, the compound is selected from Table 26.

### 7.5. Methods or Processes of Making the Conjugates

In some embodiments, set forth herein is a method of preparing an antibody-drug conjugate comprising the step of contacting a binding agent with a linker-payload compound under conditions suitable for forming a bond between the binding agent and the linker-payload compound. Also provided is a method of preparing a compound of Formula (I), Formula (Ia), Formula (Ib), or Formula (Ic) under conditions suitable for forming a bond between the binding agent and the linker-payload compound.

In certain embodiments, the antibody is reacted or treated with a reactive linker-payload to form an antibody-payload conjugate. The reaction can proceed under conditions deemed suitable by those of skill in the art. In certain embodiments, the antibody is contacted with the reactive linker-payload compound under conditions suitable for forming a bond between the antibody and the linker-payload compound. Suitable reaction conditions are well known to those in the art.

Examples of such reactions are provided in the Examples below.

In some embodiments, set forth herein is a method of making a conjugate comprising treating or contacting a compound with a binding agent under coupling conditions, wherein the compound comprises a reactive linker bonded to at least one payload moiety and, wherein the compound which reacts with a binding agent is a compound of Formula (II), Formula (IIa), Formula (IIb), or Formula (IIc) or a pharmaceutically acceptable salt, solvate, stereoisomer, or derivative thereof.

### 7.6. Pharmaceutical Compositions

Provided herein is a pharmaceutical composition a compound set forth herein, or pharmaceutically acceptable salt thereof, and a pharmaceutically acceptable excipient.

### 7.7. Methods of Using

In some embodiments, set forth herein is a method of treating a disease or disorder in a patient in need thereof comprising administering to the patient a compound or pharmaceutical composition set forth herein. In some embodiments, the administered compound is an antibody-drug conjugate set forth herein.

In some embodiments, set forth herein is a method of treating or preventing a disease, disorder, or condition selected from the group consisting of a proliferative disorder, a neurodegenerative disorder, an immunological disorder, an autoimmune disease, an inflammatory disorder, a dermatological disease, a metabolic disease, cardiovascular disease, and a gastrointestinal disease comprising administering to the subject of an effective treatment amount of a compound or pharmaceutical composition set forth herein. In some embodiments, the administered compound is an antibody-drug conjugate set forth herein.

In some embodiments, set forth herein is a method of treating a proliferative disease, a metabolic disease, inflammation, or a neurodegenerative disease in a subject comprising administering to the subject of an effective treatment amount of a compound or pharmaceutical composition set forth herein. In some embodiments, set forth herein is a method of treating a proliferative disease in a subject comprising administering to the subject of an effective treatment amount of a compound or pharmaceutical composition set forth herein. In some embodiments, the administered compound is an antibody-drug conjugate set forth herein.

In some embodiments, set forth herein is a method of treating a metabolic disease in a subject comprising administering to the subject of an effective treatment amount of a compound or pharmaceutical composition set forth herein. In some embodiments, the administered compound is an antibody-drug conjugate set forth herein.

In some embodiments, set forth herein is a method of treating inflammation in a subject comprising administering to the subject of an effective treatment amount of a compound or pharmaceutical composition of set forth herein. In some embodiments, the administered compound is an antibody-drug conjugate set forth herein.

In some embodiments, set forth herein is a method of treating a neurodegenerative disease in a subject comprising administering to the subject of an effective treatment amount of a compound or pharmaceutical composition set forth herein. In some embodiments, the administered compound is an antibody-drug conjugate set forth herein.

Although any methods and materials similar or equivalent to those described herein can be used in the practice or testing of the present invention, the preferred methods and materials are now described. All patents, applications and non-patent publications mentioned in this specification are incorporated herein by reference in their entireties.

### 8. EXAMPLES

Reagents and solvents were obtained from commercial sources such as Sinopharm Chemical Reagent Co. (SCRC), Sigma-Aldrich, Alfa, or other vendors, unless explicitly stated otherwise.

As used herein, the symbols and conventions used in these processes, schemes, and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the Examples and throughout the specification:

For the sake of conciseness, certain abbreviations are used herein. One example is the single letter abbreviation to represent amino acid residues. The amino acids and their corresponding three letter and single letter abbreviations are as follows:

### List of Abbreviations for Amino Acids

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

### List of Abbreviations

| | |
|---|---|
| TEA | triethylamin |
| THF | tetrahydrofuran |
| MeOH | Methanol |
| TEMPO | 2,2,6,6-Tetramethylpiperidine 1-oxyl |
| PhI(OAc)₂ | Diacetoxyiodo)benzene |
| PyBOP | Benzotriazol-1-yl-oxytripyrrolidino- |
| | phosphonium Hexafluorophosphate |
| HOBt | 1-Hydroxybenzotriazole |
| DIPEA | N,N-Diisopropylethylamine |
| DMF | N,N-Dimethylformamide |
| NH4C1 | Ammonium chloride |
| LiOH | Lithium hydroxide |
| HC1 | Hydrochloric acid |
| PNP | Bis(4-nitrophenyl)carbonate |
| TFA | Trifluoroacetic acid |
| K₂CO₃ | Potassium carbonate |
| MeI | Methyl iodide |
| Na₂SO₄ | Sodium sulfate |
| EtOAc | Ethyl acetate |
| MeONa | Sodium methoxide |
| prep-HPLC | Preparative high performance liquid |
| | chromatography |
| TBSCI | t-Butyldimethylchlorosilane |
| Cu(OAc)₂ | Anhydrous Cupric Acetate |
| Pb(OAc)₄ | lead tetraacetate |
| TSTU | 2-Succinimido-1,1,3,3-tetra- |
| | methyluronium tetrafluor |
| Et2N | Diethylamine |
| r.t. | room temperature |
| MS | mass spectrometry |
| ESI | electron spray ionization |
| FA | formic acid |
| NHS | N-hydroxysuccinimide |
| MTBE | methyl tert-butyl ether |
| NHS | N-hydroxysuccinimide |
| Fmoc | 9-fluorenylmethoxycarbonyl protecting |
| | group |
| DBU | 1,8-diazabicyclo[5.4.0]undec-7-ene |
| DCC | dicyclohexylcarbodiimide |
| HPLC | high pressure liquid chromatography |
| Mc-OSu | maleimidocaproyl N-hydroxysuccimidyl |
| | ester |
| DMSO | dimethylsulfoxide;. |
| MMAE | mono-methyl auristatin E or N- |
| | methylvaline-valine-dolaisoleuine- |
| | dolaproine-phenylalanine |
| AEVB | auristatin E valeryl benzylhydrazone, |
| | acid labile linker through the C-terminus |
| | of AE |

### Example 1

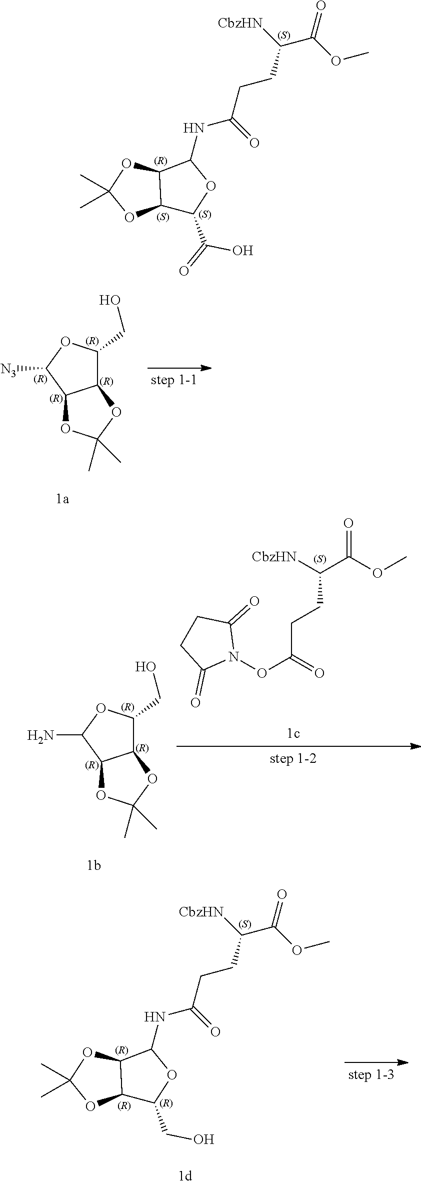

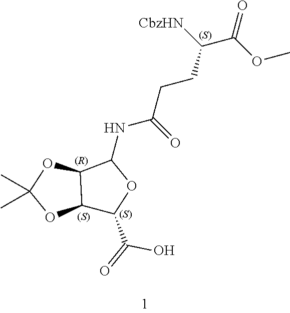

### Step 1

### (3aS,4S,6aR)-6-((S)-4-(((benzyloxy)carbonyl)amino)-5-methoxy-5-oxopentanamido)-2,2-dimethyltetrahydrofuro[3,4-d][1,3]dioxole-4-carboxylic acid (1b)

Wet Pd/C (50 mg, 10% purity) was added to a mixture of ((3aR,4R,6R,6aR)-6-azido-2,2-dimethyltetrahydrofuro[3,4-d][1,3]dioxol-4-yl)methanol 1a (400 mg, 1.75 mmol; see NUCLEOSIDES, NUCLEOTIDES AND NUCLEIC ACIDS 2018, 37, 79-88) and TEA (353.2 mg, 3.49 mmol) in THE (4 mL). The reaction mixture was purged with H₂ balloon for three times and reacted at r.t. under H₂ balloon for 2 h. After the reaction was completed, the mixture was filtered off through celite and washed with MeOH. The filtrate was concentrated under vacuum to obtain 1b (500 mg, crude).

### Step 2

### Methyl N²-((benzyloxy)carbonyl)-N⁵-((3aR,6R,6aR)-6-(hydroxymethyl)-2,2-dimethyltetrahydrofuro[3,4-d][1,3]dioxol-4-yl)-L-glutaminate (1d)

TEA (511 mg, 5.06 mmol) was added to a mixture of 1b (500 mg, crude) and 5-(2,5-dioxopyrrolidin-1-yl) 1-methyl ((benzyloxy)carbonyl)-L-glutamate 1c (550 mg, 1.40 mmol) in THE (5 mL). The reaction mixture was reacted at r.t. for overnight. After the reaction was completed, the mixture was concentrated under vacuum. The residue was purified using prep-HPLC (Method: column: XBridge Prep C18 OBD Sum 19*250 mm; Mobile phase: A—water (0.1% formic acid): B—acetonitrile; Flow rate: 20 mL/min) to afford 1d (300 mg, 45.9% yield). Exact mass calcd for C22H30N2O9 [M+H]+, 467.2; found, 467.4.

### Step 3

### (3aS,4S,6aR)-6-((S)-4-(((benzyloxy)carbonyl)amino)-5-methoxy-5-oxopentanamido)-2,2-dimethyltetrahydrofuro[3,4-d][1,3]dioxole-4-carboxylic acid (1)

TEMPO (18 mg, 0.12 mmol) and PhI(OAc)2 (285 mg, 0.88 mmol) were added to a mixture of 1d (275 mg, 0.59 mmol) in Acetonitrile/H2O (4:1, 6 mL). The reaction mixture was reacted at 40° C. for 6 h. After the reaction was completed, the mixture was concentrated under vacuum. The residue was purified using prep-HPLC (Method: column: XBridge Prep C18 OBD Sum 19*250 mm; Mobile phase: A—water (0.1% formic acid): B—acetonitrile; Flow rate: 20 mL/min) to obtain 1 (115 mg, 40% yield). Exact mass calcd for C22H28N2O10 [M+H]+, 481.17; found, 481.4

### Example 2

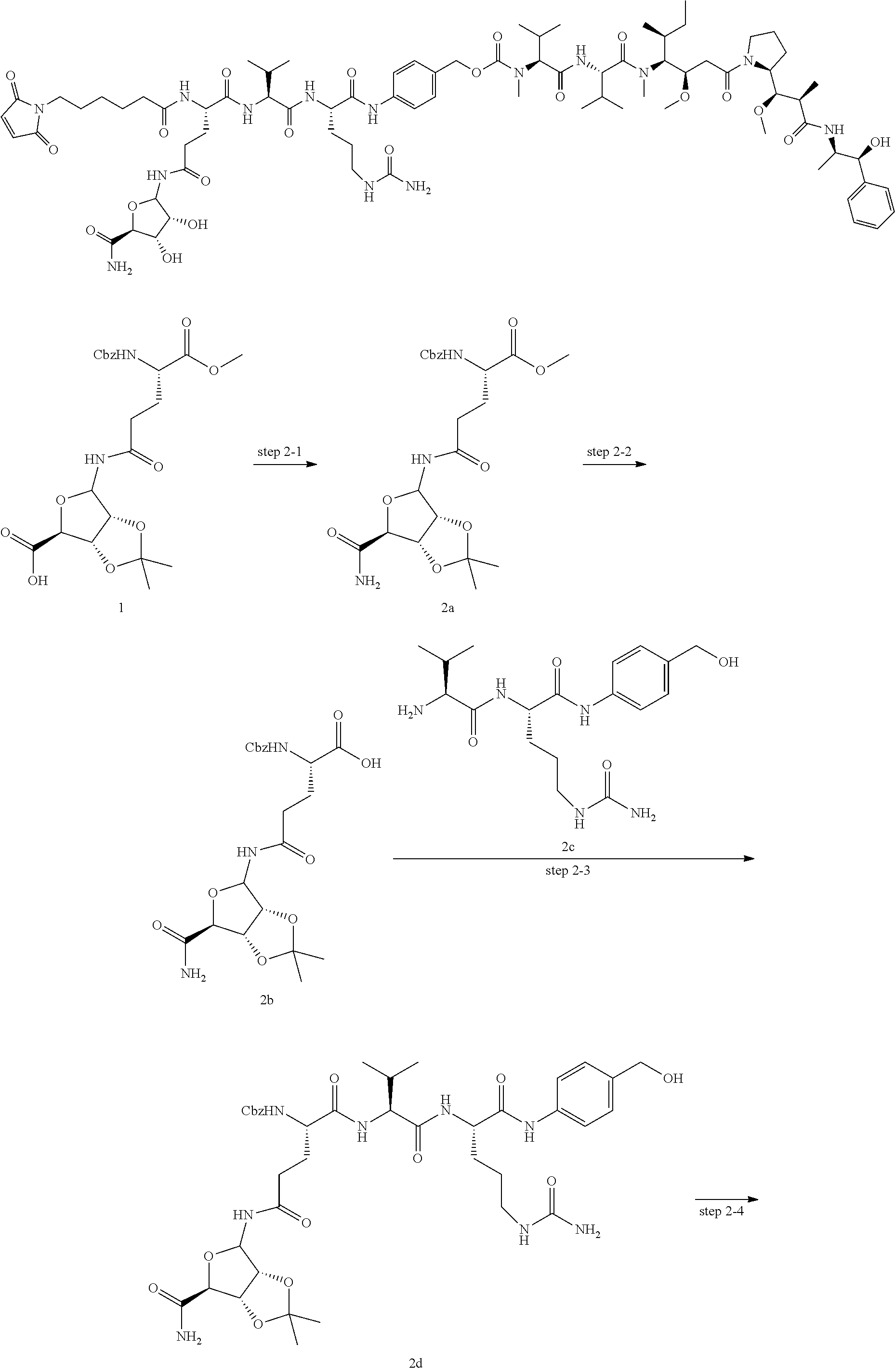

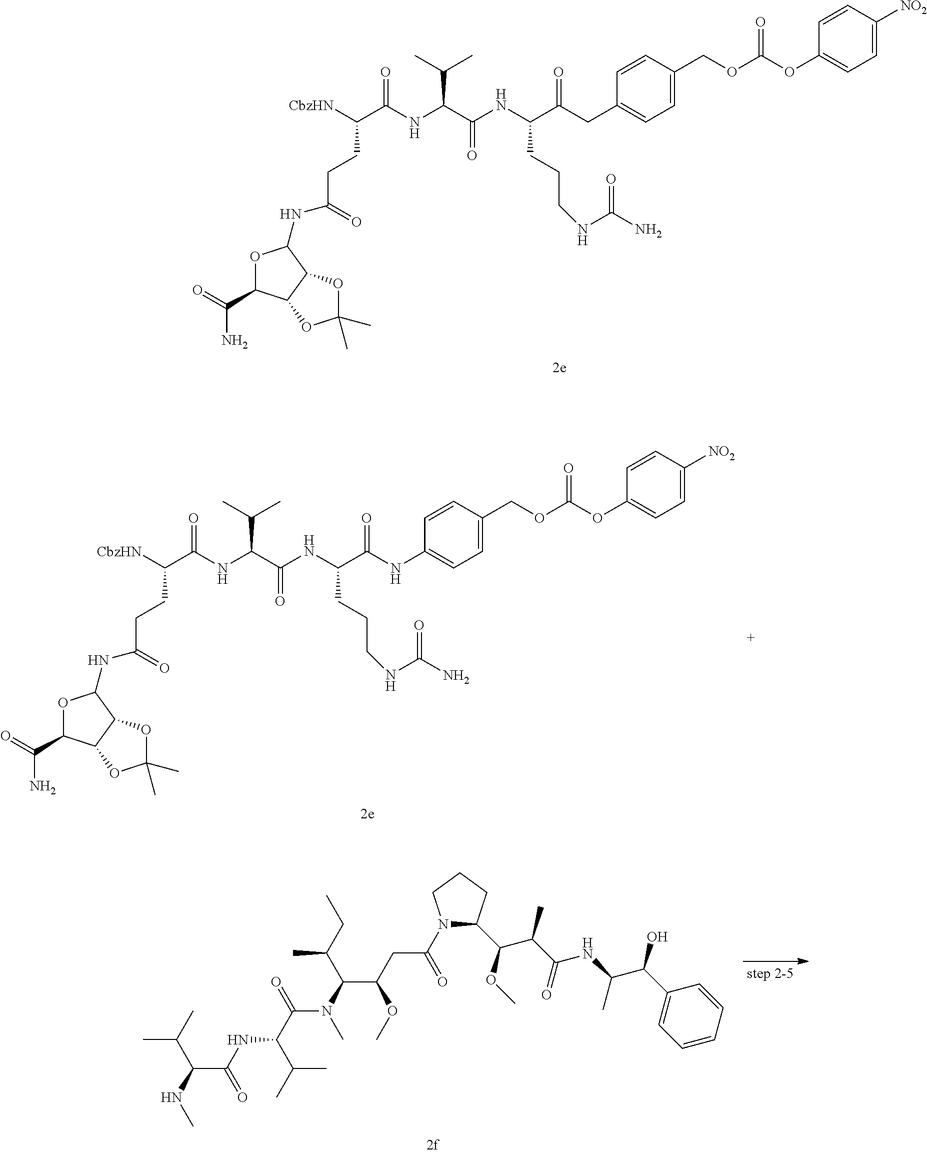 [SMILES: CC(C)[C@H](NC(=O)[C@H](CCC(=O)NC1O[C@H](C(N)=O)[C@H]2OC(C)(C)O[C@@H]12)NC(=O)OCc1ccccc1)C(=O)N[C@@H](CCCNC(N)=O)C(=O)Cc1ccc(COC(=O)Oc2ccc([N+](=O)[O-])cc2)cc1 ; CC(C)[C@H](NC(=O)[C@H](CCC(=O)NC1O[C@H](C(N)=O)[C@H]2OC(C)(C)O[C@@H]12)NC(=O)OCc1ccccc1)C(=O)N[C@@H](CCCNC(N)=O)C(=O)Nc1ccc(COC(=O)Oc2ccc([N+](=O)[O-])cc2)cc1 ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

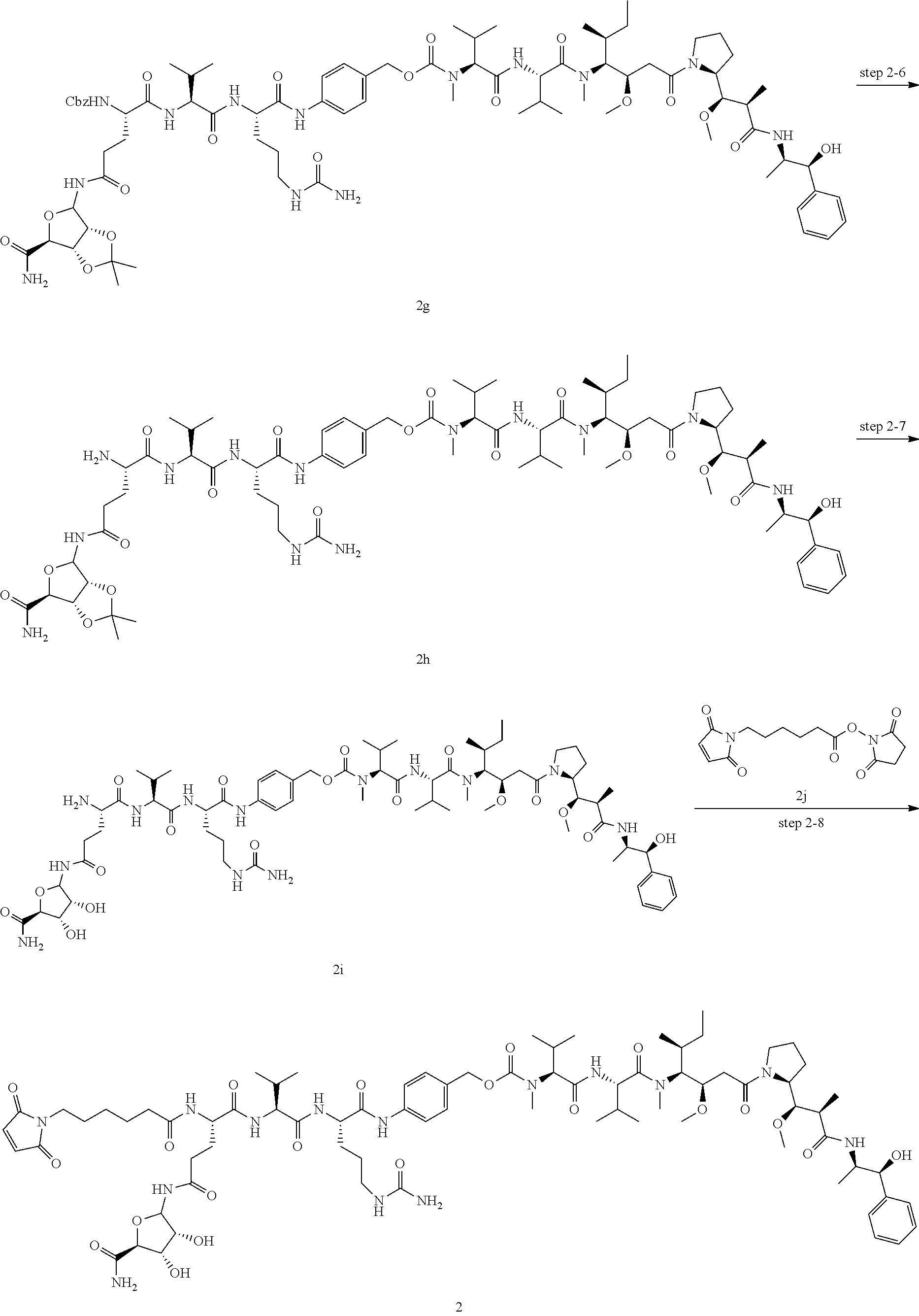

### Step 1

### Methyl N2-((benzyloxy)carbonyl)-N5-((3aR,4R,6aS)-6-carbamoyl-2,2-dimethyltetrahydrofuro [3,4-d][1,3]dioxol-4-yl)-L-glutaminate (2a)

PyBOP (433 mg, 0.83 mmol), HOBt (113 mg, 0.83 mmol) and DIEA (161 mg, 1.25 mmol) were added to a mixture of 1 (200 mg, 0.42 mmol) and NH4Cl (45 mg, 0.83 mmol) in DMF (2 mL). The reaction mixture was heated to 50° C. for overnight and monitored by LCMS. The mixture was filtered and the filtrate purified by prep-HPLC (Method: column: XBridge Prep C18 OBD 5 μm 19*250 mm; Mobile phase: A—water (0.1% formic acid): B—acetonitrile; Flow rate: 20 mL/min) to obtain 2a (112 mg, 56% yield). Exact mass calcd for C22H29N3O9 [M+H]+, 480.19; found, 480.4.

### Step 2

### N2-((benzyloxy)carbonyl)-N5-((3aR,6S,6aS)-6-carbamoyl-2,2-dimethyltetrahydrofuro[3,4-d][1,3]dioxol-4-yl)-L-glutamine (2b)

1M LiOH aqueous solution (0.36 mL, 0.36 mmol) was added to a solution of 2a (160 mg, 0.33 mmol) in THF/MeOH (4:1, 2.5 mL). The reaction mixture was reacted at r.t. for 1 h. The mixture was acidified with 1N HCl to pH=6, filtered and purified using prep-HPLC (Method: column: XBridge Prep C18 OBD Sum 19*250 mm; Mobile phase: A—water (0.1% formic acid): B—acetonitrile; Flow rate: 20 mL/min) to obtain 2b (85 mg, 65% yield). Exact mass calcd for C21H27N3O9 [M+H]+, 466.17; found, 466.4.

### Step 3

### Benzyl ((S)-5-(((3aR,6S,6aS)-6-carbamoyl-2,2-dimethyltetrahydrofuro[3,4-d][1,3]dioxol-4-yl)amino)-1-(((S)-1-(((S)-1-((4-(hydroxymethyl)phenyl)amino)-1-oxo-5-ureidopentan-2-yl)amino)-3-methyl-1-oxobutan-2-yl)amino)-1,5-dioxopentan-2-yl)carbamate (2d)

HATU (83 mg, 0.22 mmol) and DIEA (71 mg, 0.55 mmol) were added to a solution of 2b (85 mg, 0.18 mmol) in DMF (1 mL). The reaction was reacted at r.t. for 10 min then add (S)-2-((S)-2-amino-3-methylbutanamido)-N-(4-(hydroxymethyl)phenyl)-5-ureidopentanamide 2c (69 mg, 0.18 mmol; Commercially available) left at the same temperature for another 1 h. The reaction was quenched with 1 mL of water and filtered, the filtrate was purified using prep-HPLC (Method: column: XBridge Prep C18 OBD Sum 19*250 mm; Mobile phase: A—water (0.1% formic acid): B—acetonitrile; Flow rate: 20 mL/min) to obtain 2d (85 mg, 65% yield). Exact mass calcd for C39H54N8012 [M+H]+, 827.39; found, 827.6.

### Step 4

### Benzyl ((S)-5-(((3aR,6S,6aS)-6-carbamoyl-2,2-dimethyltetrahydrofuro[3,4-d][1,3]dioxol-4-yl)amino)-1-(((S)-3-methyl-1-(((S)-1-(4-((((4-nitrophenoxy)carbonyl)oxy)methyl)phenyl)-2-oxo-6-ureidohexan-3-yl)amino)-1-oxobutan-2-yl)amino)-1,5-dioxopentan-2-yl)carbamate (2e)

DIEA (40 mg, 0.31 mmol) was added to a mixture of 2d (85 mg, 0.10 mmol) and PNP (47 mg, 0.15 mmol). The reaction mixture was reacted at r.t. for 4 h and monitored by LCMS. The mixture was filtered and the filtrate was purified using prep-HPLC (Method: column: XBridge Prep C18 OBD 5 μm 19*250 mm; Mobile phase: A—water (0.1% formic acid): B—acetonitrile; Flow rate: 20 mL/min) to obtain 2e (70.2 mg, 70% yield). Exact mass calcd for C47H58N8016 [M+H]+, 991.40; found, 992.7.

### Step 5

### 4-((5S,8S,11S)-5-(3-(((3aR,6S,6aS)-6-carbamoyl-2,2-dimethyltetrahydrofuro[3,4-d][1,3]dioxol-4-yl)amino)-3-oxopropyl)-8-isopropyl-3,6,9-trioxo-1-phenyl-11-(3-ureidopropyl)-2-oxa-4,7,10-triazadodecan-12-amido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (2 g)

HOBt (4.4 mg, 0.3 mmol) and DIEA (17 mg, 0.13 mmol) were added to a mixture of 2e (65 mg, 0.07 mmol) and 2f (47 mg, 0.07 mmol; Commercially available) in DMF (1.5 mL). The reaction mixture was reacted at r.t. for 24 h. The mixture was filtered and the filtrate was purified using prep-HPLC (Method: column: XBridge Prep C18 OBD Sum 19*250 mm; Mobile phase: A—water (0.1% formic acid): B—acetonitrile; Flow rate: 20 mL/min) to obtain 2 g (68 mg, 66% yield). Exact mass calcd for C79H119N13O20 [M+H]+, 1570.87; found, 1593.2 [M+Na+].

### Step 6

### 4-((S)-2-((S)-2-((S)-2-amino-5-(3-(((3aR,6S,6aS)-6-carbamoyl-2,2-dimethyltetrahydrofuro [3,4-d][1,3]dioxol-4-yl)amino)-5-oxopentanamido)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (2 h)

Wet Pd/C (5 mg, 10% purity) was added to a solution of 2 g (23 mg, 0.01 mmol) in MeOH (1 mL). The reaction mixture was purged with H2 balloon for three times and reacted at r.t. under H2 balloon for 2 h. The reaction mixture was filtered with a syringe filter and filtrate was concentrated under vacuum to obtain 2 h (20 mg, crude). Exact mass calcd for C71H113N13O18 [M+H]+, 1436.83; found, 1438.2.

### Step 7

### 4-((S)-2-((S)-2-((S)-2-amino-5-(((3R,4S,5S)-5-carbamoyl-3,4-dihydroxytetrahydrofuran-2-yl)amino)-5-oxopentanamido)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (2i)

A mixture of 2 h (20 mg, crude) in TFA/H2O (1:1, 500 uL) was stirred at r.t. for 2 h. The mixture was filtered and filtrate was purified prep-HPLC (Method: column: XBridge Prep C18 OBD 5 μm 19*250 mm; Mobile phase: A—water (0.1% formic acid): B—acetonitrile; Flow rate: 20 mL/min) to obtain 2i (11 mg, 55% yield). Exact mass calcd for C68H109N13O18 [M+H]+, 1396.80; found, 1398.1.

### Step 8

### 4-((S)-2-((S)-2-((S)-5-(((3R,4S,5S)-5-carbamoyl-3,4-dihydroxytetrahydrofuran-2-yl)amino)-2-(6-(2,5-dioxo-2,5-dihydro-1H-pyrrol-1-yl)hexanamido)-5-oxopentanamido)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (2)

DIEA (3.1 mg, 0.02 mmol) was added to a mixture of 2i (11 mg, 0.01 mmol) and 2j (2.4 mg, 0.01 mmol) in DMF (200 uL). The reaction mixture was reacted at r.t. for 2 h. The reaction mixture was purified using prep-HPLC (Method: column: XBridge Prep C18 OBD Sum 19*250 mm; Mobile phase: A—water (0.1% formic acid): B—acetonitrile; Flow rate: 20 mL/min) to obtain 2i (11 mg, 55% yield). Exact mass calcd for C78H120N14O21 [M+H]+, 1589.88; found, 796.0 [M+2H]2+.

### Example 3

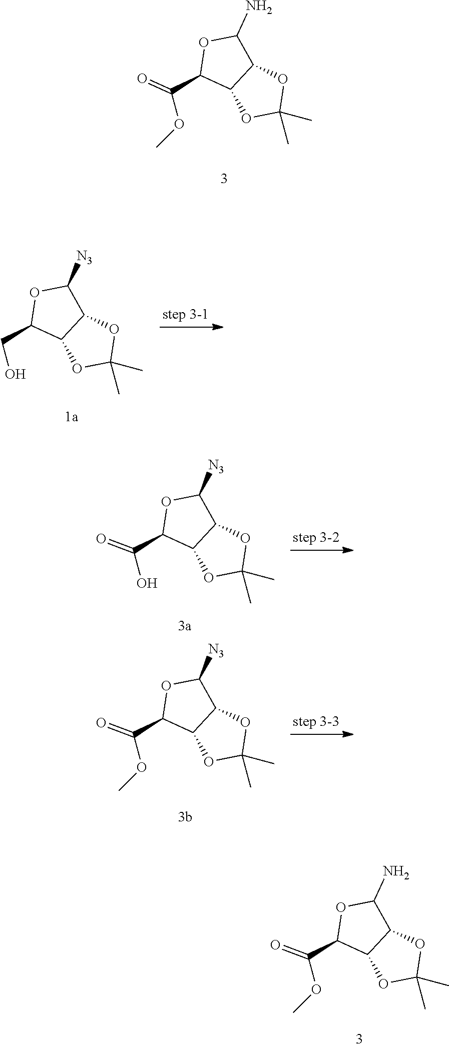

### Step 1

### (3aS,4S,6R,6aR)-6-azido-2,2-dimethyltetrahydrofuro[3,4-d][1,3]dioxole-4-carboxylic acid (3a)

3a was synthesized according to synthetic procedure of step 3 of example 1.

### Step 2

### Methyl (3aS,4S,6R,6aR)-6-azido-2,2-dimethyltetrahydrofuro[3,4-d][1,3]dioxole-4-carboxylate (3b)

K2CO3 (1.024 g, 7.41 mmol) and Mel (956 mg, 6.74 mmol) were added to a solution of 3a (1.45 g. 6.74 mmol) in DMF (10 mL). The reaction mixture was reacted at r.t. for 4 h. To the mixture was added water (20 mL), extracted with EtOAc (20 mL*3), After separation the combined organic layers were washed with brine (20 mL*3), dried over Na2SO4, filtered and the filtrate was concentrated under vacuum. The residue was purified by silica gel column flash chromatography (A—petroleum ether; B—EtOAc) to afford 3b (820 mg, 50% yield).

### Step 3

### Methyl (3aS,4S,6aR)-6-amino-2,2-dimethyltetrahydrofuro[3,4-d][1,3]dioxole-4-carboxylate (3)

Wet Pd/C (40 mg, 10% yield) was added to a solution of 3b (400 mg, 1.75 mmol) in MeOH (5 mL). The reaction mixture was purged with H2 balloon and stirred at r.t. for 2 h under H2 balloon. After the reaction was completed, the mixture was filtered off through celite and washed with MeOH. Finally concentrated under vacuum to obtain 3 (300 mg, 79% yield). Exact mass calcd for C19H15NO5 [M+H]+, 218.10; found, 218.2.

### Example 4

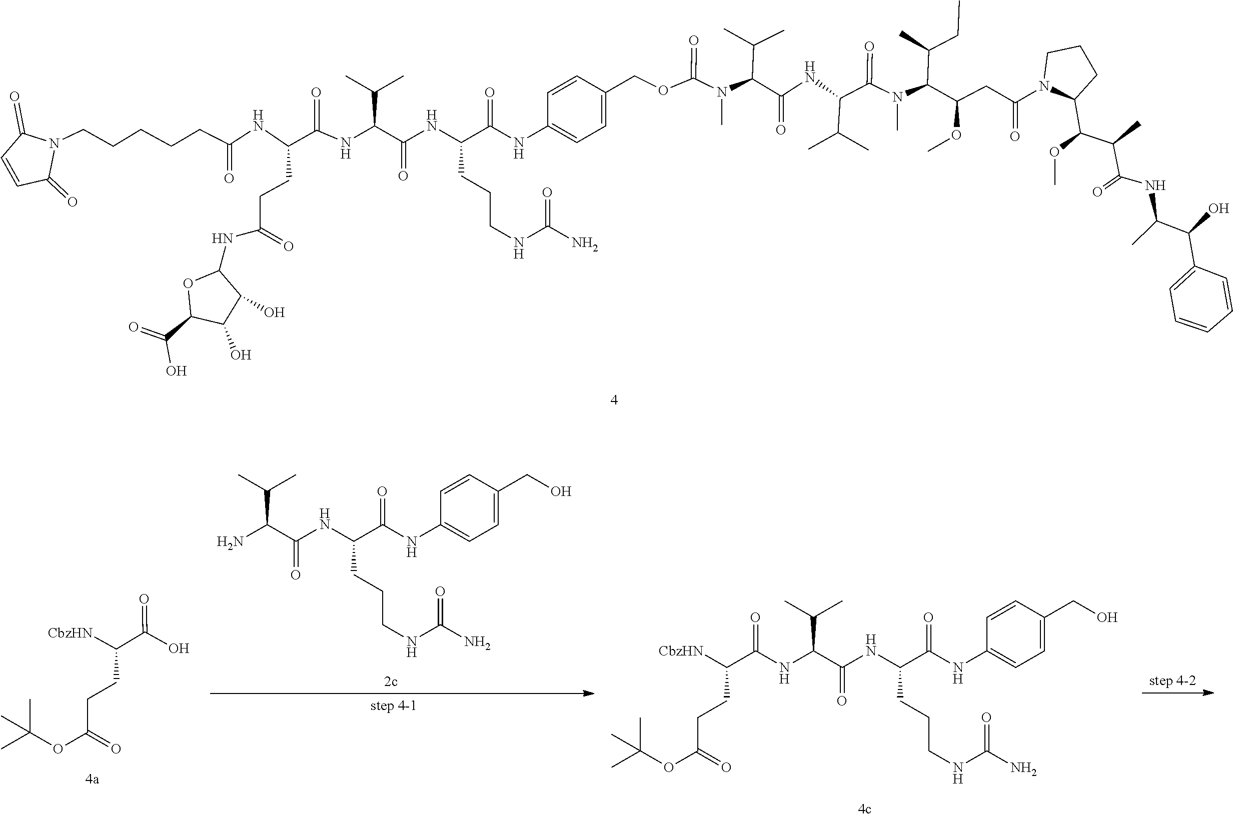 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)NC2O[C@H](C(=O)O)[C@@H](O)[C@H]2O)NC(=O)CCCCCN2C(=O)C=CC2=O)C(C)C)cc1)C(C)C ; CC(C)(C)OC(=O)CC[C@H](NC(=O)OCc1ccccc1)C(=O)O ; CC(C)[C@H](N)C(=O)N[C@@H](CCCNC(N)=O)C(=O)Nc1ccc(CO)cc1 ; CC(C)[C@H](NC(=O)[C@H](CCC(=O)OC(C)(C)C)NC(=O)OCc1ccccc1)C(=O)N[C@@H](CCCNC(N)=O)C(=O)Nc1ccc(CO)cc1]

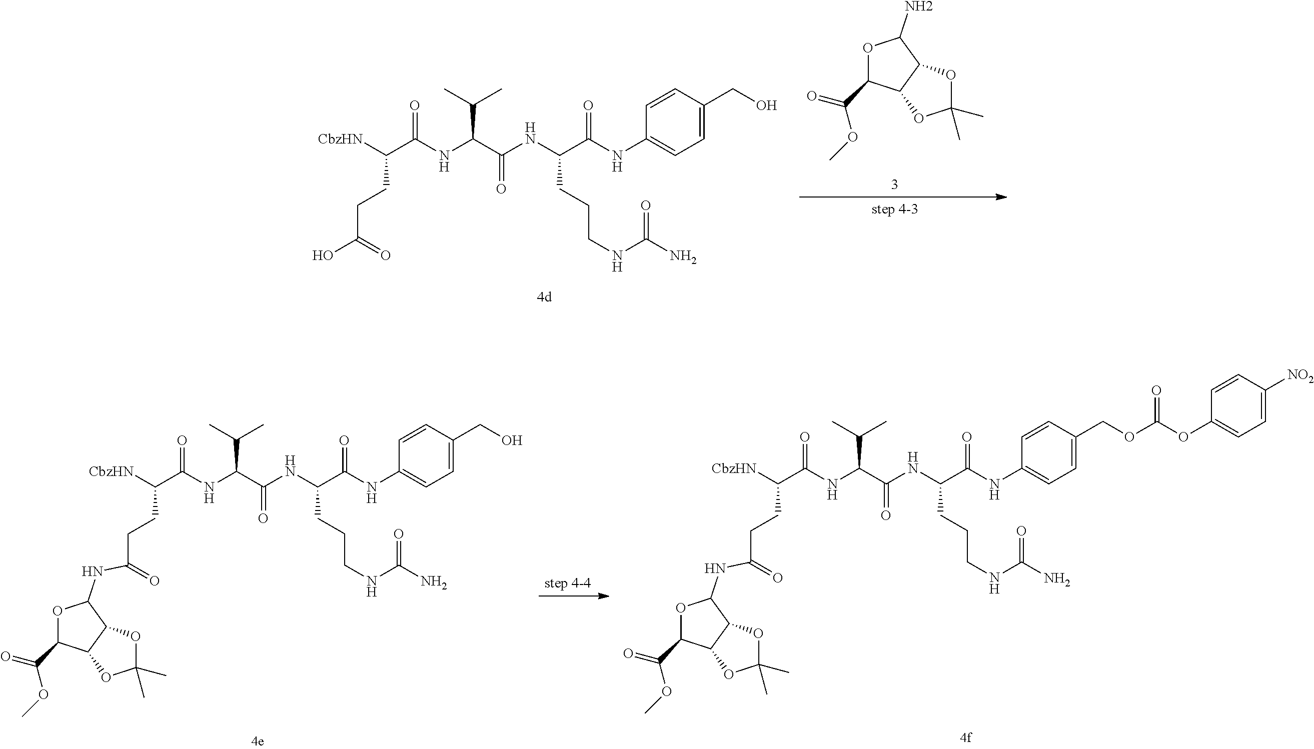 [SMILES: CC(C)[C@H](NC(=O)[C@H](CCC(=O)O)NC(=O)OCc1ccccc1)C(=O)N[C@@H](CCCNC(N)=O)C(=O)Nc1ccc(CO)cc1 ; COC(=O)[C@H]1OC(N)[C@@H]2OC(C)(C)O[C@H]12 ; COC(=O)[C@H]1OC(NC(=O)CC[C@H](NC(=O)OCc2ccccc2)C(=O)N[C@H](C(=O)N[C@@H](CCCNC(N)=O)C(=O)Nc2ccc(CO)cc2)C(C)C)[C@@H]2OC(C)(C)O[C@H]12 ; COC(=O)[C@H]1OC(NC(=O)CC[C@H](NC(=O)OCc2ccccc2)C(=O)N[C@H](C(=O)N[C@@H](CCCNC(N)=O)C(=O)Nc2ccc(COC(=O)Oc3ccc([N+](=O)[O-])cc3)cc2)C(C)C)[C@@H]2OC(C)(C)O[C@H]12]

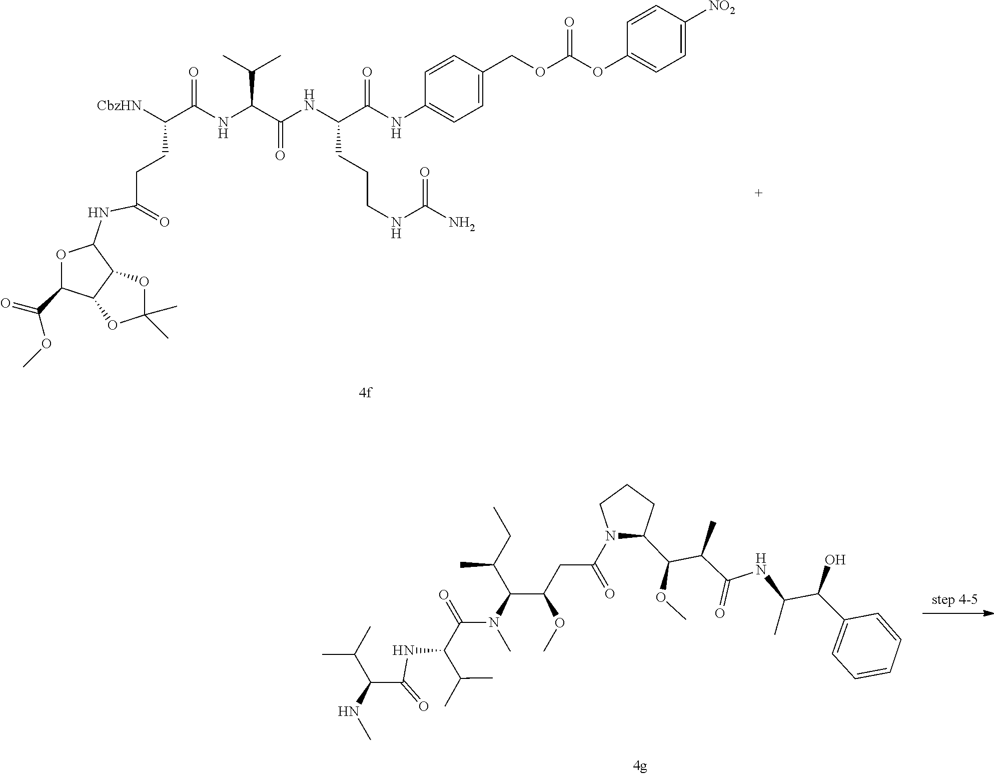 [SMILES: COC(=O)[C@H]1OC(NC(=O)CC[C@H](NC(=O)OCc2ccccc2)C(=O)N[C@H](C(=O)N[C@@H](CCCNC(N)=O)C(=O)Nc2ccc(COC(=O)Oc3ccc([N+](=O)[O-])cc3)cc2)C(C)C)[C@@H]2OC(C)(C)O[C@H]12 ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

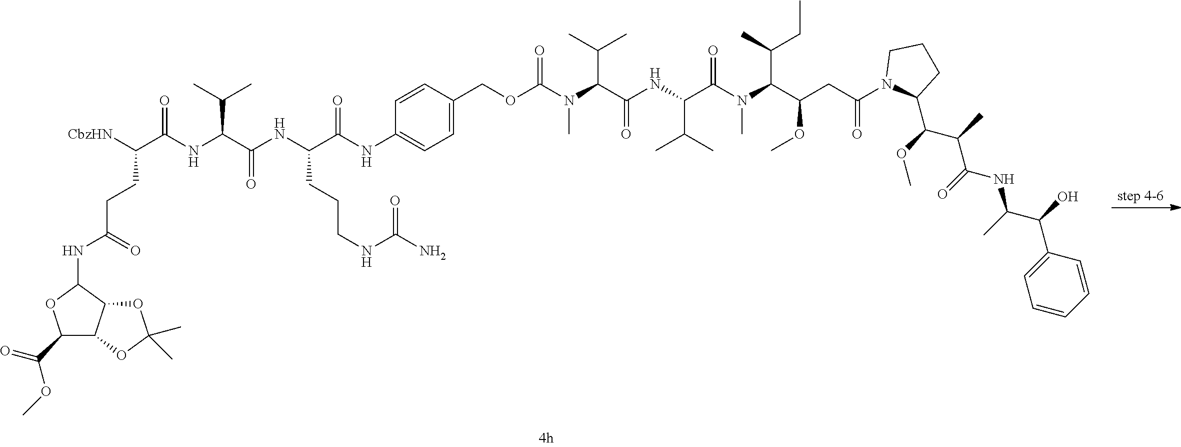 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)NC2O[C@H](C(=O)OC)[C@H]3OC(C)(C)O[C@@H]23)NC(=O)OCc2ccccc2)C(C)C)cc1)C(C)C]

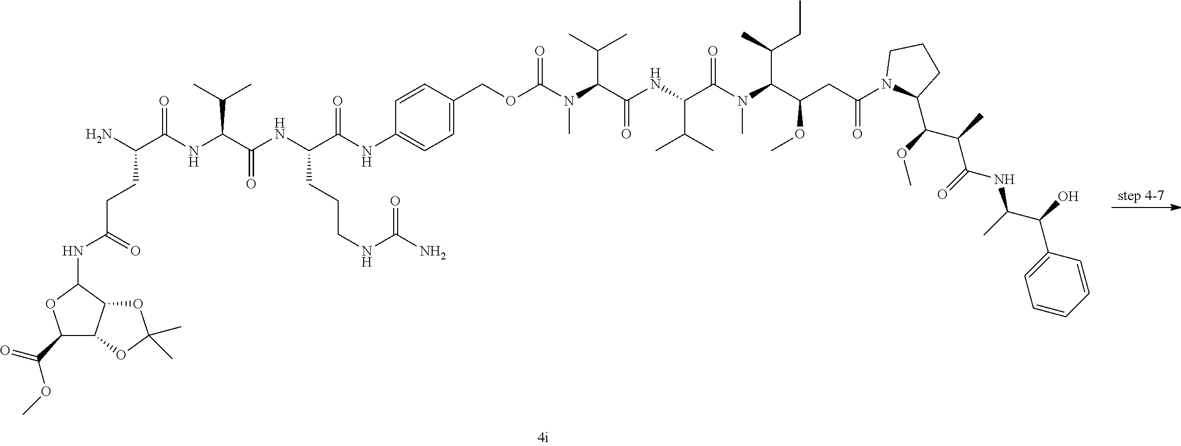 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@@H](N)CCC(=O)NC2O[C@H](C(=O)OC)[C@H]3OC(C)(C)O[C@@H]23)C(C)C)cc1)C(C)C]

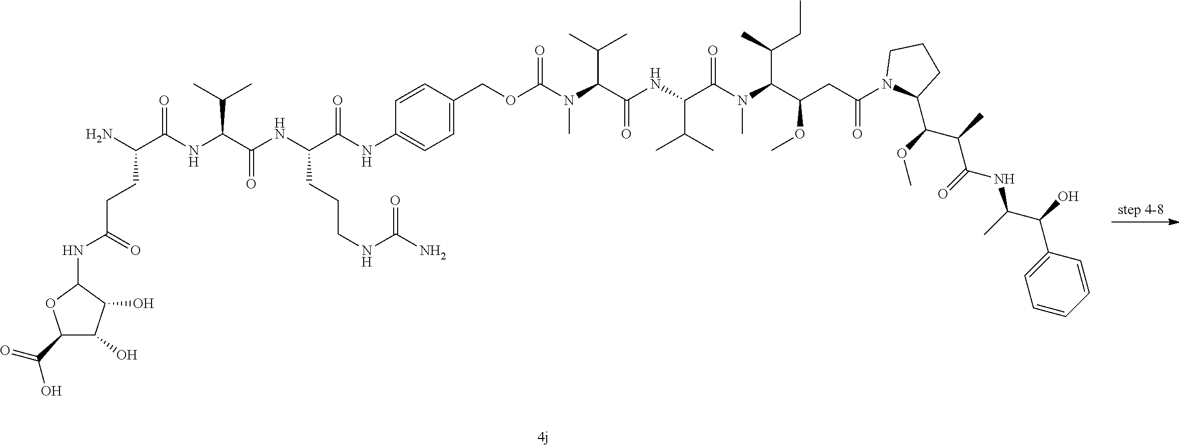 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@@H](N)CCC(=O)NC2O[C@H](C(=O)O)[C@@H](O)[C@H]2O)C(C)C)cc1)C(C)C]

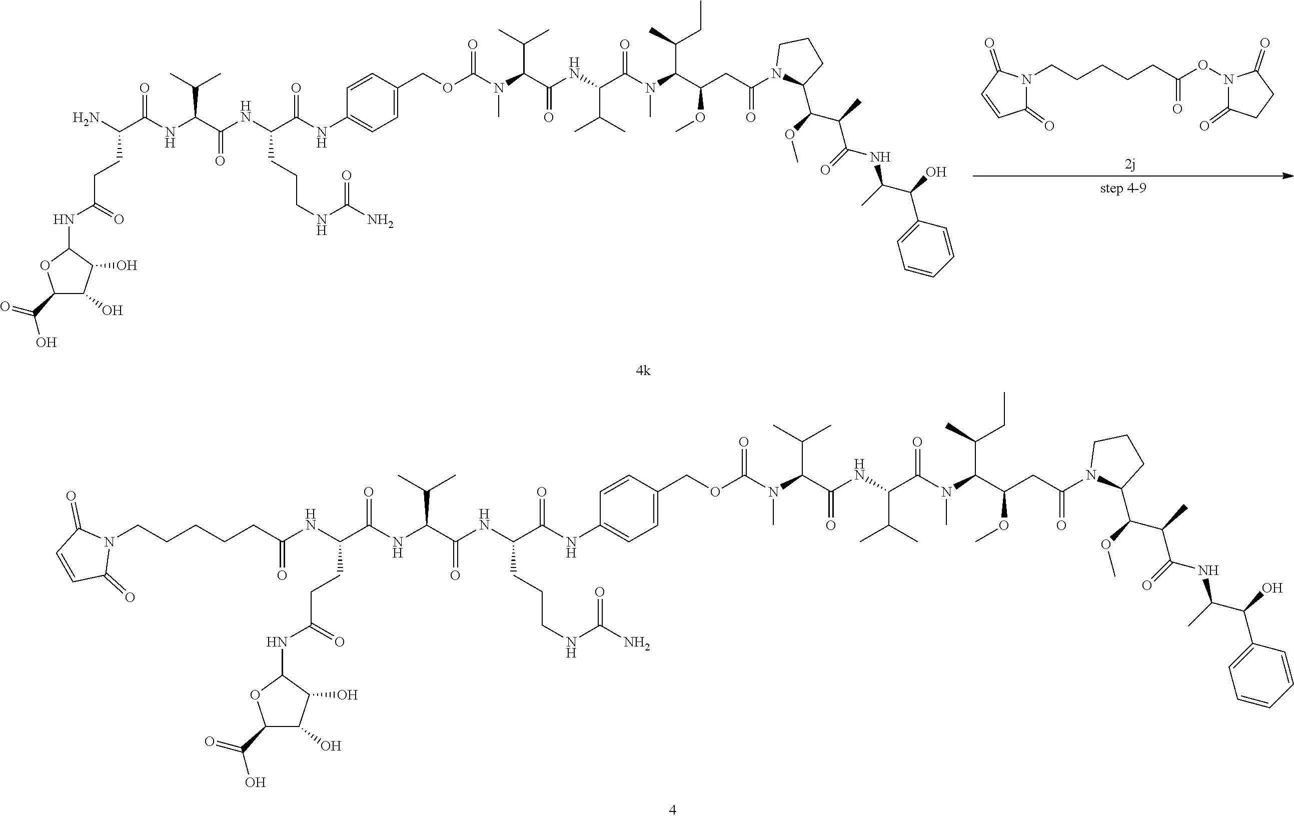

### Step 1

Tert-butyl (S)-4-(((benzyloxy)carbonyl)amino)-5-(((S)-1-(((S)-1-((4-(hydroxymethyl)phenyl)amino)-1-oxo-5-ureidopentan-2-yl)amino)-3-methyl-1-oxobutan-2-yl)amino)-5-oxopentanoate (4c)

HATU (338 mg, 0.89 mmol) and DIEA (345 mg, 2.67 mmol) were added to a mixture of 4a (300 mg, 0.89 mmol) in DMF (5 mL). The reaction mixture was reacted at r.t. for 10 min. Then added 2c (100 mg, 0.89 mmol) left at the same temperature for another 2 h. The reaction was quenched with 10 mL of water and filtered, the cake was triturated with EtOAc at r.t. Filtered again and dry the filter cake under vacuum to obtain 4c (520 mg, 84% yield).

### Step 2

### (S)-4-(((benzyloxy)carbonyl)amino)-5-(((S)-1-(((S)-1-((4-(hydroxymethyl)phenyl)amino)-1-oxo-5-ureidopentan-2-yl)amino)-3-methyl-1-oxobutan-2-yl)amino)-5-oxopentanoic acid (4d)

A mixture of 4c (300 mg, 0.43 mmol) in TFA/DCM (1:1, 4 mL) was stirred at r.t. for 2 h. The mixture was concentrated under vacuum to provide a residue then dissolved in MeOH (3 mL) and cooled to 0° C., MeONa (200 mg) was added to the methanol solution, and stirred at 0° C. for 30 min. The mixture was acidified with 1M HCl to pH=3-4. Most of precipitated. filtered and the cake washed with water. Finally dry the filter cake under vacuum to afford 4d (140 mg, 51% yield). Exact mass calcd for C31H42N6O9 [M+H]+, 643.30; found, 643.5.

### Step 3

### Methyl (3aS,4S,6aR)-6-((S)-4-(((benzyloxy)carbonyl)amino)-5-(((S)-1-(((S)-1-((4-(hydroxymethyl)phenyl)amino)-1-oxo-5-ureidopentan-2-yl)amino)-3-methyl-1-oxobutan-2-yl)amino)-5-oxopentanamido)-2,2-dimethyltetrahydrofuro[3,4-d][1,3]dioxole-4-carboxylate (4e)

HATU (99 mg, 0.22 mmol) and DIEA (84 mg, 0.65 mmol) were added to a mixture of 4d (140 mg, 0.22 mmol) and 3 (47.32 mg, 0.22 mmol) in DMF (1.2 mL). The mixture was reacted at r.t. for 1 h. The mixture was filtered and purified using prep-HPLC (Method: column: XBridge Prep C18 OBD Sum 19*250 mm; Mobile phase: A—water (0.1% formic acid): B—acetonitrile; Flow rate: 20 mL/min) to obtain 4e (92 mg, 49% yield). Exact mass calcd for C40H55N7O13 [M+H]+, 842.39; found, 842.6 [M+H]+.

### Step 4

### Methyl (3aS,4S,6R,6aR)-6-((S)-4-(((benzyloxy)carbonyl)amino)-5-(((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)amino)-5-oxopentanamido)-2,2-dimethyltetrahydrofuro[3,4-d][1,3]dioxole-4-carboxylate (4f)

4f (37 mg, 88% yield) was synthesized according to synthetic procedure of step 4 of example 2. Exact mass calcd for C47H58N8017 [M+H]+, 1007.39; found, 1007.6.

### Step 5

### Methyl (3aS,4S,6aR)-6-((S)-4-(((benzyloxy)carbonyl)amino)-5-(((S)-1-(((S)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanamido)-2,2-dimethyltetrahydrofuro[3,4-d][1,3]dioxole-4-carboxylate (4 h)

4 h (40 mg, 68% yield) was synthesized according to synthetic procedure of step 5 of example 2. Exact mass calcd for C80H120N12O21 [M+H]+, 1586.87; found, 1587.37.

### Step 6

### Methyl (3aS,4S,6aR)-6-((S)-4-amino-5-(((S)-1-(((S)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanamido)-2,2-dimethyltetrahydrofuro[3,4-d][1,3]dioxole-4-carboxylate (4i)

4i (18 mg, crude) was synthesized according to synthetic procedure of step 6 of example 2. Exact mass calcd for C72H114N12O19 [M+H]+, 1451.83; found, 1453.2.

### Step 7

### Methyl (2S,3S,4R)-5-((S)-4-amino-5-(((S)-1-(((S)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanamido)-3,4-dihydroxytetrahydrofuran-2-carboxylate (4j)

4j (10 mg, 70% yield) was synthesized according to synthetic procedure of step 7 of example 2. Exact mass calcd for C69H110N12O19 [M+H]+, 1411.80; found, 1413.2.

### Step 8

### (2S,3S,4R)-5-((S)-4-amino-5-(((S)-1-(((S)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanamido)-3,4-dihydroxytetrahydrofuran-2-carboxylic acid (4k)

1M LiOH aqueous solution (0.2 mL, 0.2 mmol) was added to a solution of 4j (10 mg, 0.007 mmol) in MeOH (0.5 mL). The reaction mixture was reacted at r.t. for 30 min. The mixture was acidified with 1N HCl to pH=6, filtered and purified using prep-HPLC (Method: column: XBridge Prep C18 OBD 5 μm 19*250 mm; Mobile phase: A—water (0.1% formic acid): B—acetonitrile; Flow rate: 20 mL/min) to obtain 4k (5.6 mg, 57% yield). Exact mass calcd for C68H108N12O19 [M+H]+, 1397.79; found, 1399.2.

### Step 9

### (2S,3S,4R)-5-((S)-5-(((S)-1-(((S)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-4-(6-(2,5-dioxo-2,5-dihydro-1H-pyrrol-1-yl)hexanamido)-5-oxopentanamido)-3,4-dihydroxytetrahydrofuran-2-carboxylic acid (4)

4 (4.3 mg, 67% yield) was synthesized according to synthetic procedure of step 7 of example 2. Exact mass calcd for C78H118N13O22 [M+H]+, 1590.86; found, 1591.8.

### Example 5

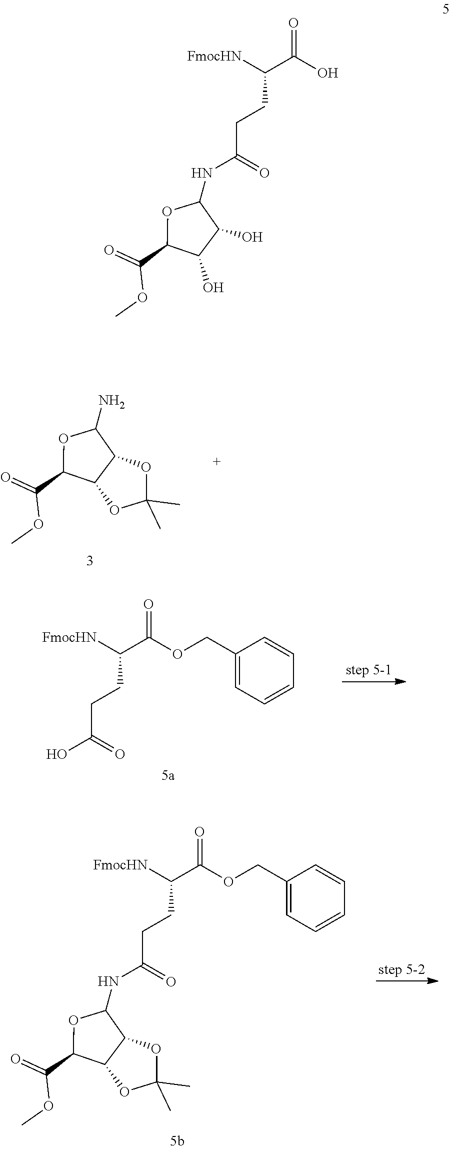

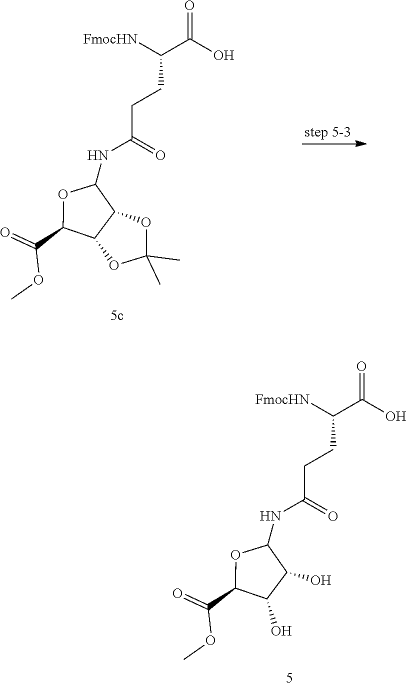

### Step 1

### Methyl (3aS,4S,6R)-6-((S)-4-((((9H-fluoren-9-yl)methoxy)carbonyl)amino)-5-(benzyloxy)-5-oxopentanamido)-2,2-dimethyltetrahydrofuro[3,4-d][1,3]dioxole-4-carboxylate (5b)

HATU (580 mg, 1.52 mmol) and DIEA (591 mg, 4.57 mmol) were added to a mixture of (S)-4-((((9H-fluoren-9-yl)methoxy)carbonyl)amino)-5-(benzyloxy)-5-oxopentanoic acid 5a (700 mg, 1.52 mmol) and 3 (320 mg, 1.47 mmol). The reaction mixture was reacted at r.t. for 2 h. The reaction mixture was quenched with 20 mL of water, extracted with EtOAc (30 Ml*3). After separation, the combined organic washed with brine (20 mL*3), dried over Na2SO4, filtered and the filtrate was concentrated under vacuum. The residue was purified using silica gel flash chromatography (A—Petroleum ether; B—EtOAc) to obtain 5b (742.7 mg, 74% yield). Exact mass calcd for C36H38N2010 [M+H]+, 659.25; found, 659.5.

### Step 2

### N2-(((9H-fluoren-9-yl)methoxy)carbonyl)-N5-((3aR,6S,6aS)-6-(methoxycarbonyl)-2,2-dimethyltetrahydrofuro[3,4-d][1,3]dioxol-4-yl)-L-glutamine (5c)

Wet Pd/C (70 mg, 10% purity) was added to a solution of 5b (739 mg, 1.12 mmol) in MeOH (10 mL). The reaction mixture was purged with H2 balloon for three times then stirred at r.t. for 2 h under H2 balloon. The mixture was filtered off through celite washed MeOH, the filtrate was concentrated under vacuum to obtain 5c (600 mg, 94% yield). Exact mass calcd for C29H32N2010 [M+H]+, 569.21; found, 569.5.

### Step 3

### N2-(((9H-fluoren-9-yl)methoxy)carbonyl)-N5-((3R,4S,5S)-3,4-dihydroxy-5-(methoxycarbonyl)tetrahydrofuran-2-yl)-L-glutamine (5)

5 (35 mg, 47% yield) was synthesized according to synthetic procedure of step 7 of example 2. Exact mass calcd for C26H28N2010 [M+H]+, 529.17; found, 529.4.

### Example 6

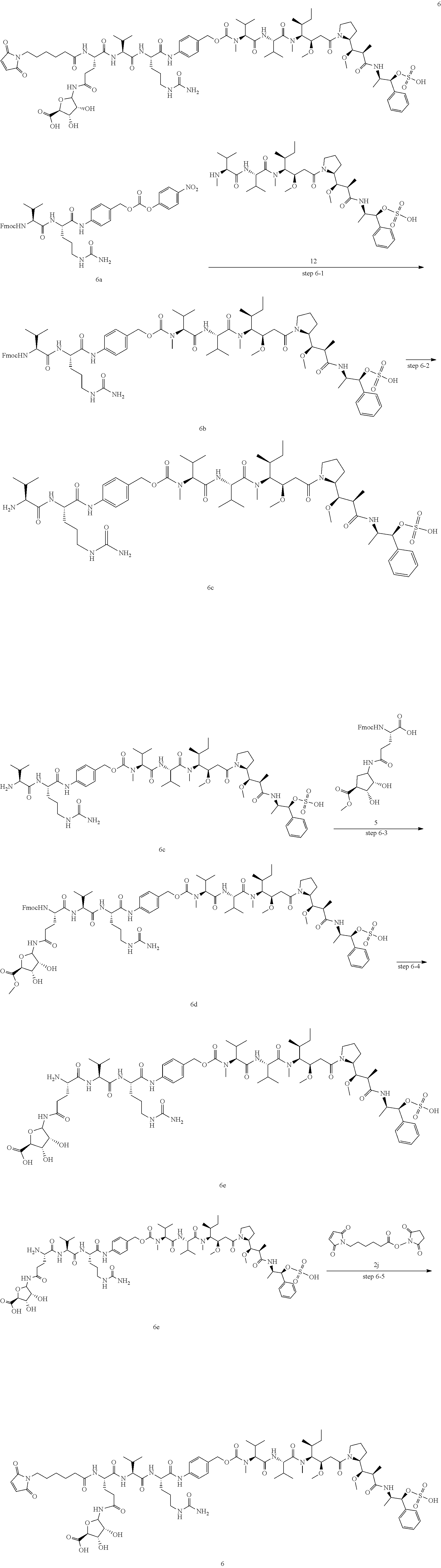

### Step 1

### (1S,2R)-2-((2R,3R)-3-((S)-1-((5S,8S,11S,12R)-1-(4-((S)-2-((S)-2-((((9H-fluoren-9-yl)methoxy)carbonyl)amino)-3-methylbutanamido)-5-ureidopentanamido)phenyl)-11-((S)-sec-butyl)-5,8-diisopropyl-12-methoxy-4,10-dimethyl-3,6,9-trioxo-2-oxa-4,7,10-triazatetradecan-14-oyl)pyrrolidin-2-yl)-3-methoxy-2-methylpropanamido)-1-phenylpropyl hydrogen sulfate (6b)

6b (50 mg, 50% yield) was synthesized according to synthetic procedure of step 5 of example 2. Exact mass calcd for C73H104N10017S [M−H]−, 1423.73; found, 1425.0.

### Step 2

### (1S,2R)-2-((2R,3R)-3-((S)-1-((5S,8S,11S,12R)-1-(4-((S)-2-((S)-2-amino-3-methylbutanamido)-5-ureidopentanamido)phenyl)-11-((S)-sec-butyl)-5,8-diisopropyl-12-methoxy-4,10-dimethyl-3,6,9-trioxo-2-oxa-4,7,10-triazatetradecan-14-oyl)pyrrolidin-2-yl)-3-methoxy-2-methylpropanamido)-1-phenylpropyl hydrogen sulfate (6c)

A solution of 6b (50 mg, 0.04 mmol) in 20% piperidine (1 mL, solution in DMF) was stirred at r.t. for 10 min. The mixture was acidified with 1N HCl to pH=5-6 then filtered and purified using prep-HPLC (Method: column: XBridge Prep C18 OBD Sum 19*250 mm; Mobile phase: A—water (0.1% formic acid): B—acetonitrile; Flow rate: 20 mL/min) to obtain 6c (34 mg, 81% yield). Exact mass calcd for C58H94N10015S [M−H]−, 1201.66; found, 1201.8.

### Step 3

### Methyl (2S,3S,4R)-5-((S)-4-((((9H-fluoren-9-yl)methoxy)carbonyl)amino)-5-(((S)-1-(((S)-1-((4-((5S,8S,11S,12R)-11-((S)-sec-butyl)-5,8-diisopropyl-12-(2-((S)-2-((1R,2R)-1-methoxy-2-methyl-3-oxo-3-(((1S,2R)-1-phenyl-1-(sulfooxy)propan-2-yl)amino)propyl)pyrrolidin-1-yl)-2-oxoethyl)-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanamido)-3,4-dihydroxytetrahydrofuran-2-carboxylate (6d)

HATU (15 mg, 0.04 mmol) and DIEA (15 mg, 0.119 mmol) were added to a solution of 5 (21 mg, 0.04 mmol) in DMF (2 mL). The reaction mixture was reacted at r.t. for 10 min. Then added 6c (33.5 mg, 0.028 mmol) left at the same temperature for another 1 h. The mixture was filtered and the filtrate was purified by using prep-HPLC (Method: column: XBridge Prep C18 OBD Sum 19*250 mm; Mobile phase: A—water (0.1% formic acid): B—acetonitrile; Flow rate: 20 mL/min) to obtain 6c (34 mg, 81% yield). Exact mass calcd for C84H120N12O24S [M−H]−, 1711.83; found, 1713.2.

### Step 4

### (2S,3S,4R)-5-((S)-4-amino-5-(((S)-1-(((S)-1-((4-((5S,8S,11S,12R)-11-((S)-sec-butyl)-5,8-diisopropyl-12-(2-((S)-2-((1R,2R)-1-methoxy-2-methyl-3-oxo-3-(((1S,2R)-1-phenyl-1-(sulfooxy)propan-2-yl)amino)propyl)pyrrolidin-1-yl)-2-oxoethyl)-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanamido)-3,4-dihydroxytetrahydrofuran-2-carboxylic acid (6e)

2M LiOH aqueous solution (20 uL, 0.04 mmol) was added to a solution of 6d (25 mg, 0.01 mmol) in THE (0.2 mL). The reaction mixture was reacted at r.t. for 2 h. The mixture was acidified with 1N HCl to pH=6, filtered and purified using prep-HPLC (Method: column: XBridge Prep C18 OBD 5 μm 19*250 mm; Mobile phase: A—water (0.1% formic acid): B—acetonitrile; Flow rate: 20 mL/min) to obtain 6e (85 mg, 48% yield). Exact mass calcd for C68H108N12O22S [M−H]−, 1475.74; found, 1475.9.

### Step 5

### (2S,3S,4R)-5-((S)-5-(((S)-1-(((S)-1-((4-((5S,8S,11S,12R)-11-((S)-sec-butyl)-5,8-diisopropyl-12-(2-((S)-2-((1R,2R)-1-methoxy-2-methyl-3-oxo-3-(((1S,2R)-1-phenyl-1-(sulfooxy)propan-2-yl)amino)propyl)pyrrolidin-1-yl)-2-oxoethyl)-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-4-(6-(2,5-dioxo-2,5-dihydro-1H-pyrrol-1-yl)hexanamido)-5-oxopentanamido)-3,4-dihydroxytetrahydrofuran-2-carboxylic acid (6)

6 (3.6 mg, 80% yield) was synthesized according to synthetic procedure of step 8 of example 2. Exact mass calcd for C78H119N13O25S [M−H]−, 1668.82; found, 1670.7.

### Example 7

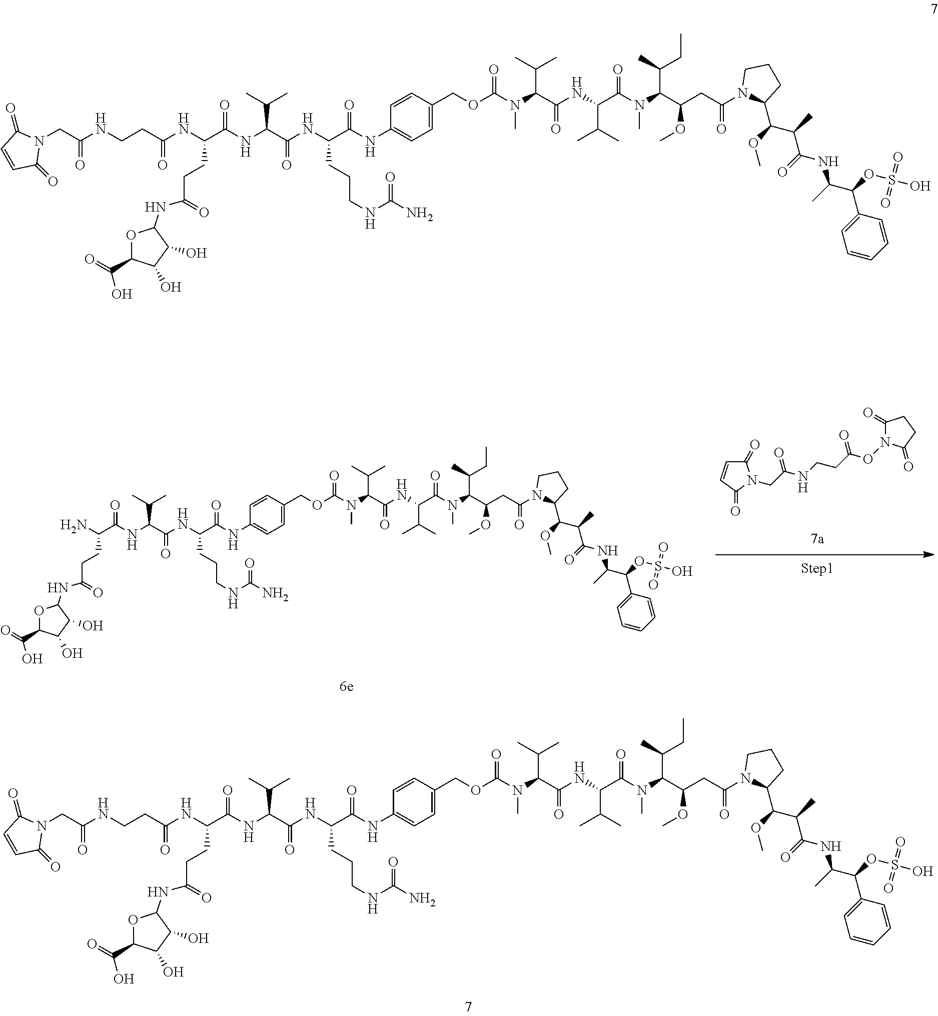

### Step 1

### (2S,3S,4R)-5-((S)-5-(((S)-1-(((S)-1-((4-((5S,8S,11S,12R)-11-((S)-sec-butyl)-5,8-diisopropyl-12-(2-((S)-2-((1R,2R)-1-methoxy-2-methyl-3-oxo-3-(((1S,2R)-1-phenyl-1-(sulfooxy)propan-2-yl)amino)propyl)pyrrolidin-1-yl)-2-oxoethyl)-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-4-(3-(2-(2,5-dioxo-2,5-dihydro-1H-pyrrol-1-yl)acetamido)propanamido)-5-oxopentanamido)-3,4-dihydroxytetrahydrofuran-2-carboxylic acid (7)

Compound 7 (4.5 mg, 57% yield) was synthesized according to synthetic procedure of step 8 of example 2.

MS (ESI) m/z: 1684.8 [M−H]−

### Example 8

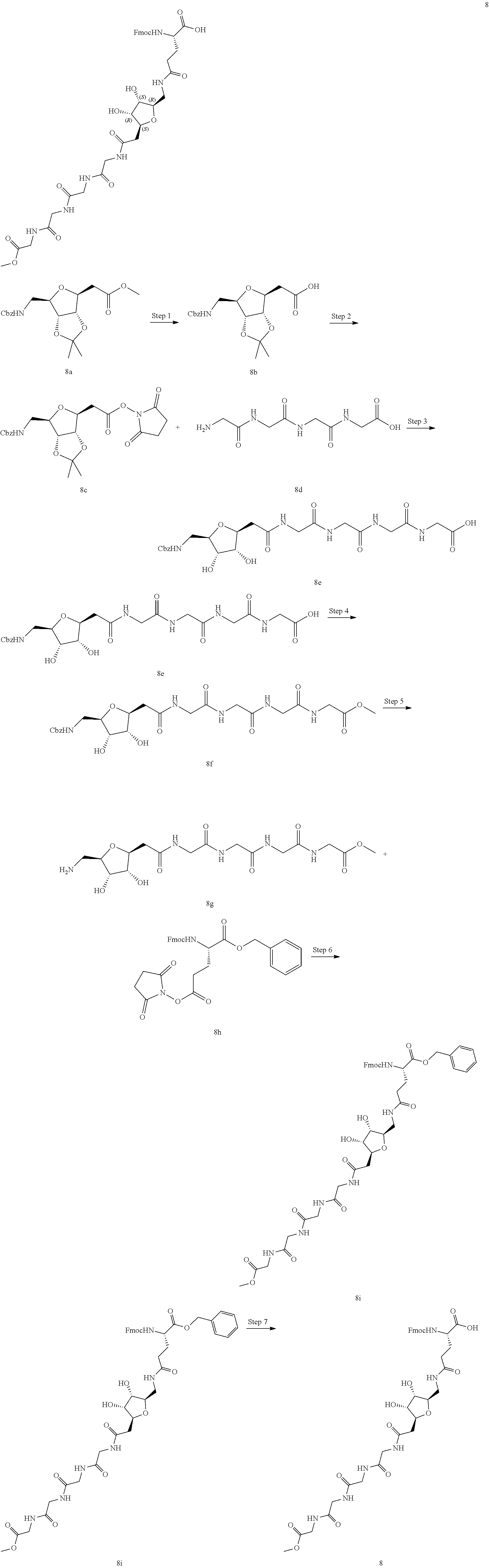

### Step 1

### 2-((3aS,4S,6R,6aR)-6-((((benzyloxy)carbonyl)amino)methyl)-2,2-dimethyltetrahydrofuro[3,4-d][1,3]dioxol-4-yl)acetic acid (8b)

8a (740 mg, 1.95 mmol, synthesized according to the procedure described in Journal of Carbohydrate Chemistry 2000, 19, 653-657) was dissolved in a mixed solvent of MeOH (4 mL) and H2O (1 mL) followed by the addition of NaOH (187 mg, 23.95 mmol). The resulting mixture was stirred at r.t. for 16 h. After complete reaction, the mixture was concentrated under reduced pressure to remove most of MeOH. Then H2O (5 mL) was added and the mixture was adjusted to pH=5-6 with aq. HCl (1 M) at 0° C. The mixture was extracted with EA (20 mL*3). The combined organic layers were dried over anhydrous Na2SO4, filtered and concentrated under reduced pressure to afford 8b as a colorless oil (560 mg, 79% yield).

MS (ESI) m/z: 364.2 [M−H]−

### Step 2

### 2,5-dioxopyrrolidin-1-yl 2-((3aS,4S,6R,6aR)-6-((((benzyloxy)carbonyl)amino)methyl)-2,2-dimethyltetrahydrofuro[3,4-d][1,3]dioxol-4-yl)acetate (8c)

8b was dissolved in DCM (5 mL) followed by the addition of DCC (365 mg, 1.77 mmol) and HOSu (204 mg, 1.77 mmol). The resulting mixture was stirred at 25° C. for 2 h. After complete reaction, the reaction mixture was filtered and concentrated to afford a yellow solid as the crude product which was purified by flash column (eluted with PE/EA). 8c was obtained as a pale yellow solid (490 mg, 72% yield).

MS (ESI) m/z: 485.4 [M+Na]+

### Step 3

### (2-((2S,3R,4S,5R)-5-((((benzyloxy)carbonyl)amino)methyl)-3,4-dihydroxytetrahydrofuran-2-yl)acetyl)glycylglycylglycylglycine (8e)

8c (490 mg, 1.06 mmol) was dissolved in THE (8 mL) followed by addition of 8d (381.70 mg, 1.06 mmol, commercially available) and saturated aq. NaHCO₃ (1 mL). The resulting mixture was stirred at 25° C. for 2 h. After complete reaction, the mixture was adjusted to pH=1 at 0° C. with aq. HCl (1 M) and purified by Prep HPLC (Method: column: XBridge Prep C18 OBD Sum 19*250 mm; Mobile phase: A—water (0.1% formic acid): B—acetonitrile; Flow rate: 20 mL/min) to afford 8e as the final product (100 mg, 19% yield).

MS (ESI) m/z: 594.4 [M+H]+

### Step 4

### Methyl (2-((2S,3R,4S,5R)-5-((((benzyloxy)carbonyl)amino)methyl)-3,4-dihydroxytetrahydrofuran-2-yl)acetyl)glycylglycylglycylglycinate (8f)

8e (90 mg, 0.16 mmol) was dissolved in DMF (2 mL) followed by the successive addition of CH3I (35 mg, 0.24 mmol) and NaHCO₃ (27 mg, 0.33 mmol). The resulting mixture was stirred at 25° C. for 16 h. After complete reaction, the reaction mixture was adjusted to pH=6 at 0° C. with aq. HCl (1 M) and purified by Prep HPLC (Method: column: XBridge Prep C18 OBD Sum 19*250 mm; Mobile phase: A—water (0.1% formic acid): B—acetonitrile; Flow rate: 20 mL/min). After lyophilization, 8f was obtained as a clear oil (85 mg, 83% yield).

MS (ESI) m/z: 590.4 [M+Na]+

### Step 5

### Methyl (2-((2S,3R,4S,5R)-5-(aminomethyl)-3,4-dihydroxytetrahydrofuran-2-yl)acetyl)glycylglycylglycylglycinate (8 g)

8f (85 mg, 0.15 mmol) was dissolved in MeOH (2 mL) followed by the addition of Pd/C (wet, 10 mg, 10%). The resulting mixture was stirred at 25° C. for 2 h. After complete reaction, the reaction mixture was filtrated with diatomite. The obtained filtrate was concentrated under reduced pressure to afford a clear oil as the crude product (65 mg) which was used directly in next step.

MS (ESI) m/z: 434.3 [M+H]+

### Step 6

### Benzyl N2-(((9H-fluoren-9-yl)methoxy)carbonyl)-N5-(((2R,3S,4R,5S)-3,4-dihydroxy-5-(2-((2-((2-((2-((2-methoxy-2-oxoethyl)amino)-2-oxoethyl)amino)-2-oxoethyl)amino)-2-oxoethyl)amino)-2-oxoethyl)tetrahydrofuran-2-yl)methyl)-L-glutaminate (8i)

8 g (65 mg, 0.15 mmol) was dissolved in THE (2 mL) followed by the addition of 8 h (100 mg, 0.18 mmol, commercially available) and saturated NaHCO₃ (0.5 mL). The resulting mixture was stirred at 25° C. for 2 h. After complete reaction, the reaction mixture was concentrated under reduced pressure to afford 8i (132 mg, crude) which was used directly in next step.

MS (ESI) m/z: 875.5 [M+H]+

### Step 7

N2-(((9H-fluoren-9-yl)methoxy)carbonyl)-N5-(((2R,3S,4R,5S)-3,4-dihydroxy-5-(2-((2-((2-((2-((2-methoxy-2-oxoethyl)amino)-2-oxoethyl)amino)-2-oxoethyl)amino)-2-oxoethyl)amino)-2-oxoethyl)tetrahydrofuran-2-yl)methyl)-L-glutamine (8)

Compound 8 (70 mg, 59% yield) was synthesized according to synthetic procedure of step 2 of example 5.

MS (ESI) m/z: 785.5 [M+H]+

### Example 9

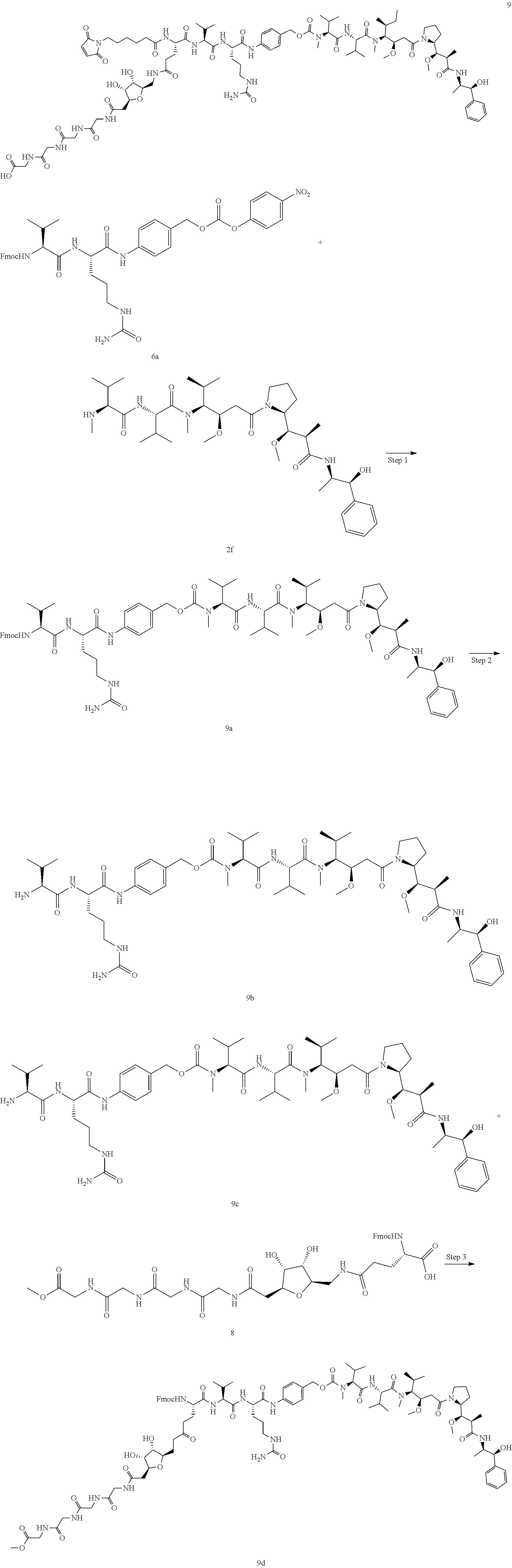

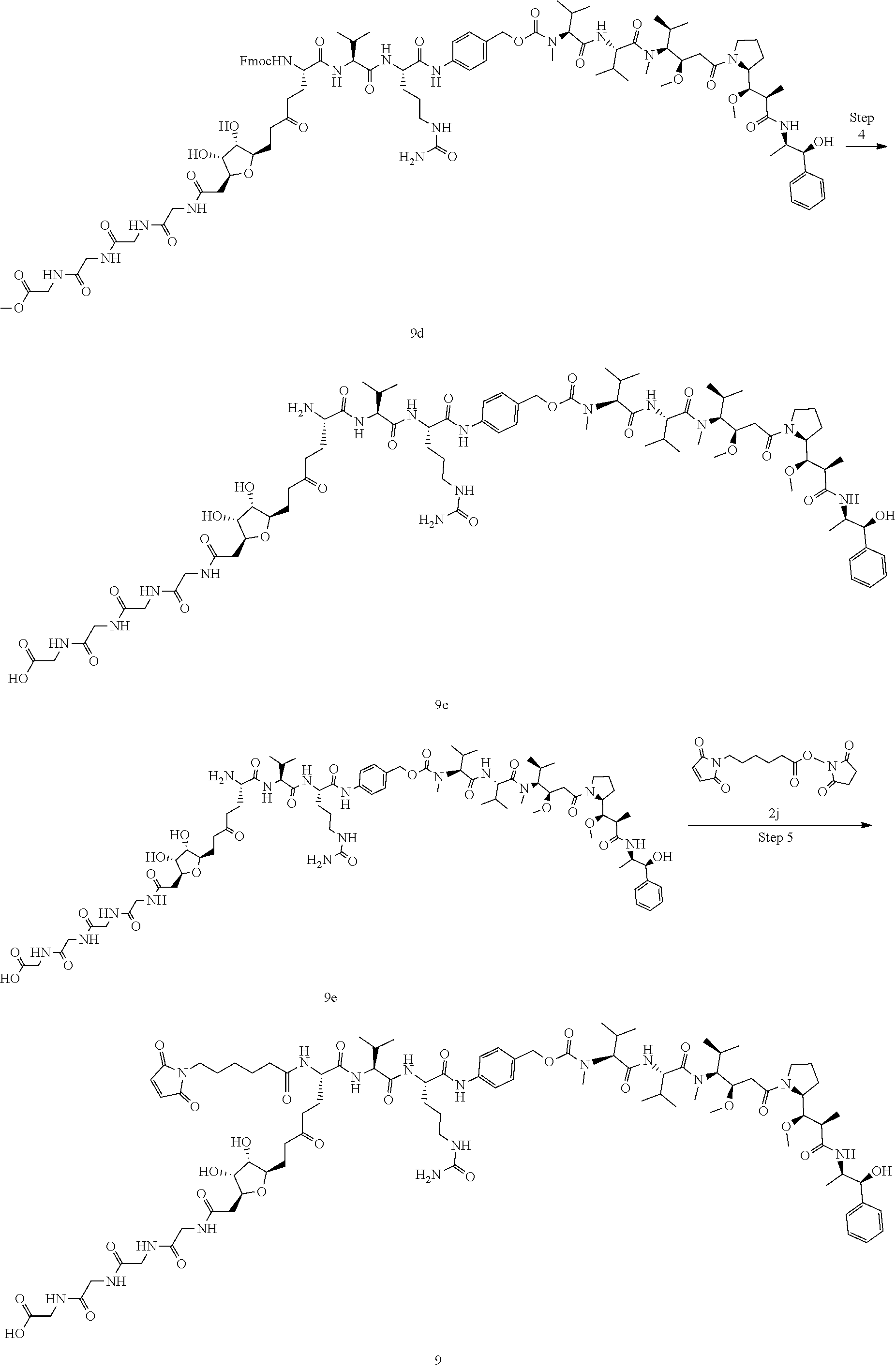

### Step 1

### 4-((S)-2-((S)-2-((((9H-fluoren-9-yl)methoxy)carbonyl)amino)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (9a)

9a (60 mg, 32% yield) was synthesized according to synthetic procedure of step 5 of example 2.

MS (ESI) m/z: 1347.1 [M+H]+

### Step 2

### 4-((S)-2-((S)-2-amino-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (9b)

9b (31 mg, 70% yield) was synthesized according to synthetic procedure of step 2 of example 6.

MS (ESI) m/z: 1124.7 [M+H]+

### Step 3

### Methyl (2-((2S,3R,4S,5R)-5-(((S)-4-((((9H-fluoren-9-yl)methoxy)carbonyl)amino)-5-(((S)-1-(((S)-1-((4-(((((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamoyl)oxy)methyl)phenyl)amino)-1-oxo-5-ureidopentan-2-yl)amino)-3-methyl-1-oxobutan-2-yl)amino)-5-oxopentanamido)methyl)-3,4-dihydroxytetrahydrofuran-2-yl)acetyl)glycylglycylglycylglycinate (9d)

9d (19 mg, 51% yield) was synthesized according to synthetic procedure of step 3 of example 6.

MS (ESI) m/z: 946.1 [M+2H]2+

### Step 4

### (2-((2S,3R,4S,5R)-5-(((S)-4-amino-5-(((S)-1-(((S)-1-((4-(((((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamoyl)oxy)methyl)phenyl)amino)-1-oxo-5-ureidopentan-2-yl)amino)-3-methyl-1-oxobutan-2-yl)amino)-5-oxopentanamido)methyl)-3,4-dihydroxytetrahydrofuran-2-yl)acetyl)glycylglycylglycylglycine (9e)

9d (19 mg, 0.01 mmol) was dissolved in a mixed solvent of CH3CN and H2O (v:v=1:1, 1 mL) followed by the addition of piperidine (1.12 g, 2.936 mmol). The resulting mixture was stirred at 25° C. for 2 h. Then the reaction mixture was adjusted to pH=6 with HCl (1 M) at 0° C. and purified by Prep HPLC (Method: column: XBridge Prep C18 OBD 5 μm 19*250 mm; Mobile phase: A—water (0.1% formic acid): B—acetonitrile; Flow rate: 20 mL/min) to afford 9e (14 mg, 87% yield).

MS (ESI) m/z: 828.0 [M+2H]2+

### Step 5

### (2-((2S,3R,4S,5R)-5-(((S)-4-(6-(2,5-dioxo-2,5-dihydro-1H-pyrrol-1-yl)hexanamido)-5-(((S)-1-(((S)-1-((4-(((((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamoyl)oxy)methyl)phenyl)amino)-1-oxo-5-ureidopentan-2-yl)amino)-3-methyl-1-oxobutan-2-yl)amino)-5-oxopentanamido)methyl)-3,4-dihydroxytetrahydrofuran-2-yl)acetyl)glycylglycylglycylglycine (9)

Compound 9 (13 mg, 83% yield) was synthesized according to synthetic procedure of step 8 of example 2.

MS (ESI) m/z: 924.5 [M+2H]2+

### Example 10

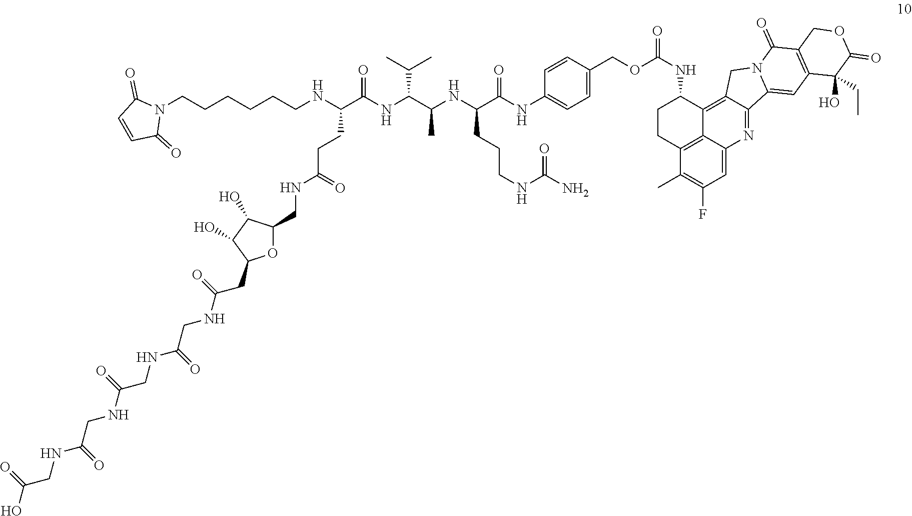 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@@H](CCCNC(N)=O)N[C@@H](C)[C@H](NC(=O)[C@H](CCC(=O)NC[C@H]2O[C@@H](CC(=O)NCC(=O)NCC(=O)NCC(=O)NCC(=O)O)[C@H](O)[C@@H]2O)NCCCCCCN2C(=O)C=CC2=O)C(C)C)cc1)CC3]

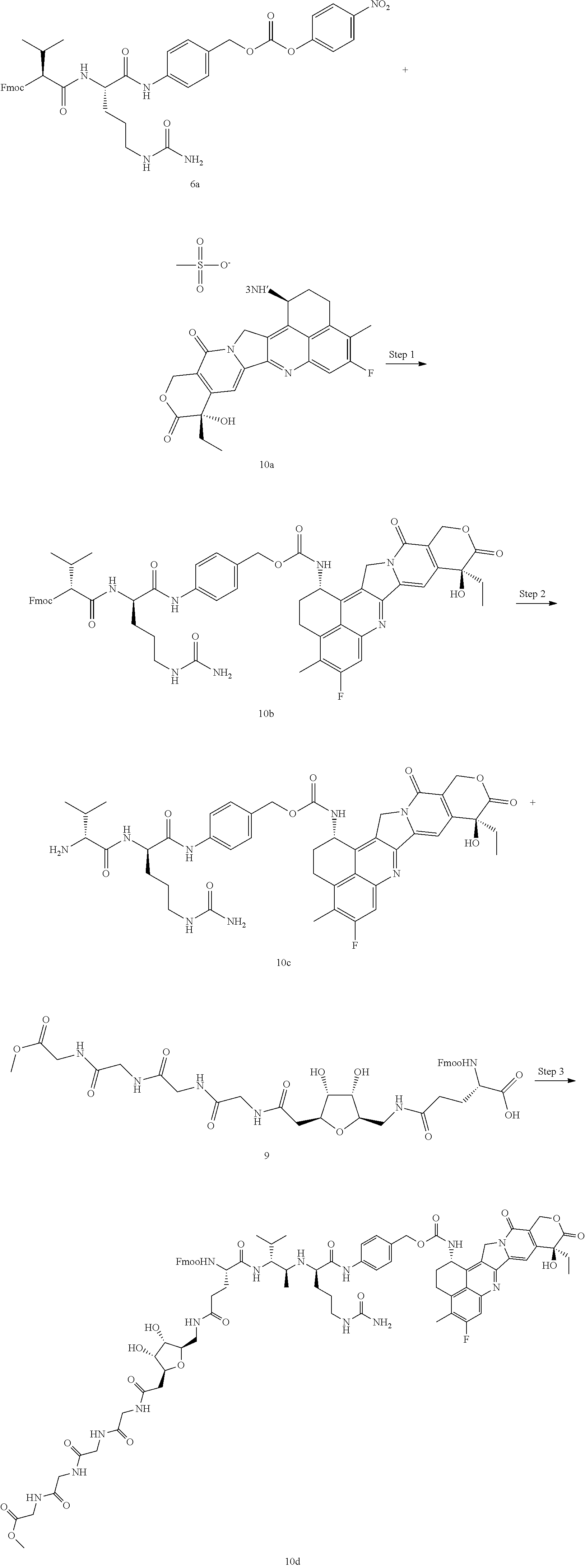

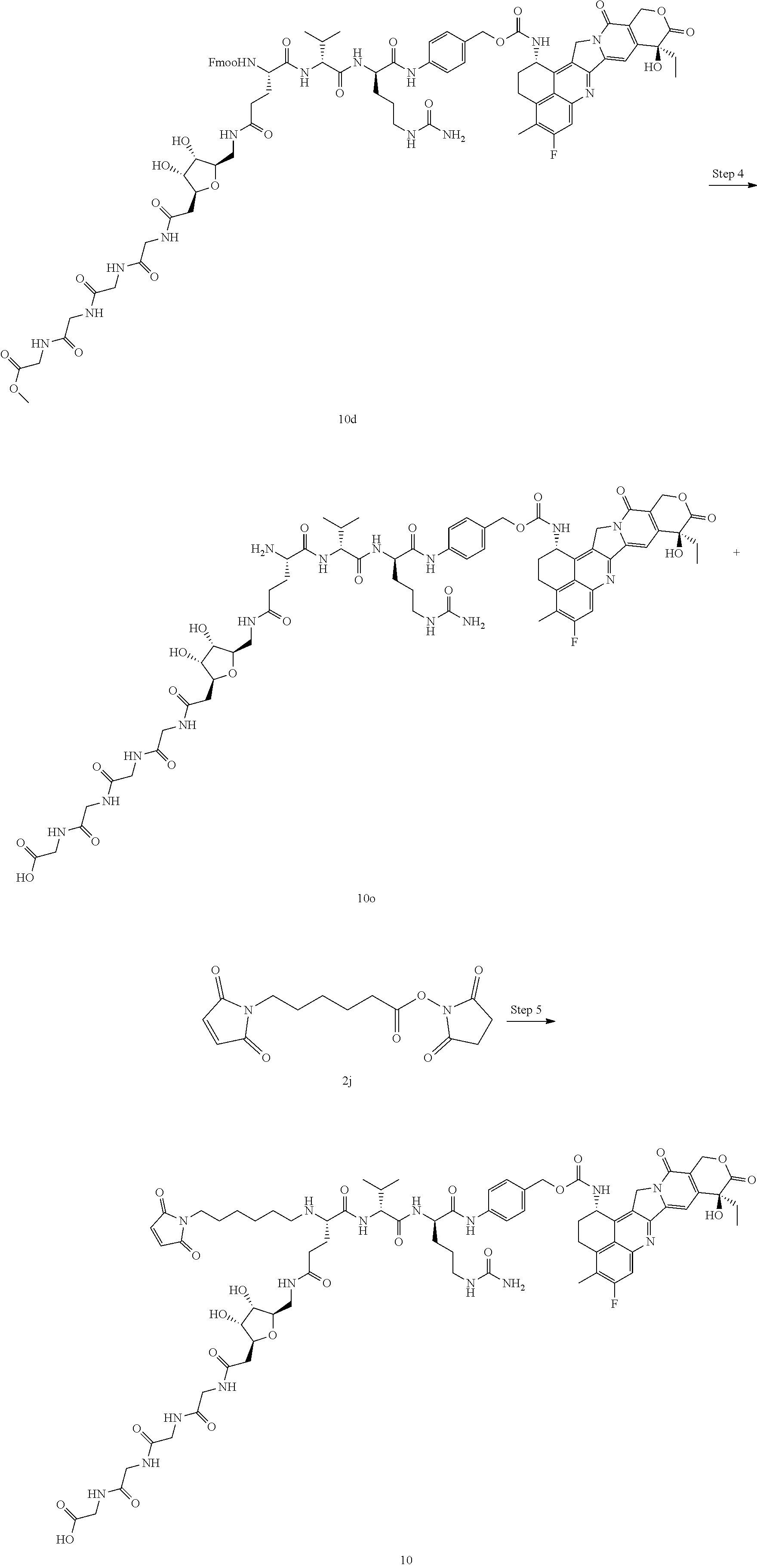

### Step 1

### 4-((R)-2-((R)-2-((((9H-fluoren-9-yl)methoxy)carbonyl)amino)-3-methylbutanamido)-5-ureidopentanamido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (2b)

6a (100 mg, 0.13 mmol) and 10a (69 mg, 0.13 mmol) were dissolved in DMF (1 mL) followed by the addition of DIEA (51 mg, 0.39 mmol). The resulting mixture was stirred at 25° C. for 4 h. After complete reaction, the reaction mixture was concentrated under reduced pressure to afford the title compound 10b (200 mg, crude).

MS (ESI) m/z: 1063.6 [M+H]+

### Step 2

### 4-((R)-2-((R)-2-amino-3-methylbutanamido)-5-ureidopentanamido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (10c)

10c (52 mg, 66% yield) was synthesized according to synthetic procedure of step 2 of example 6.

### Step 3

### Methyl (2-((2S,3R,4S,5R)-5-(((S)-4-((((9H-fluoren-9-yl)methoxy)carbonyl)amino)-5-(((R)-1-(((R)-1-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1-oxo-5-ureidopentan-2-yl)amino)-3-methyl-1-oxobutan-2-yl)amino)-5-oxopentanamido)methyl)-3,4-dihydroxytetrahydrofuran-2-yl)acetyl)glycylglycylglycylglycinate (10d)

10d (28 mg, 28% yield) was synthesized according to synthetic procedure of step 3 of example 6.

MS (ESI) m/z: 804.0 [M+2H]2+

### Step 4

### (2-((2S,3R,4S,5R)-5-(((S)-4-amino-5-(((R)-1-(((R)-1-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1-oxo-5-ureidopentan-2-yl)amino)-3-methyl-1-oxobutan-2-yl)amino)-5-oxopentanamido)methyl)-3,4-dihydroxytetrahydrofuran-2-yl)acetyl)glycylglycylglycylglycine (10e)

10e (16 mg, 68% yield) was synthesized according to synthetic procedure of step 4 of example 9.

MS (ESI) m/z: 1372.9 [M+H]+

### Step 5

### (2-((2S,3R,4S,5R)-5-(((S)-4-(6-(2,5-dioxo-2,5-dihydro-1H-pyrrol-1-yl)hexanamido)-5-(((R)-1-(((R)-1-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)amino)-1-oxo-5-ureidopentan-2-yl)amino)-3-methyl-1-oxobutan-2-yl)amino)-5-oxopentanamido)methyl)-3,4-dihydroxytetrahydrofuran-2-yl)acetyl)glycylglycylglycylglycine (10)

Compound 10 (7.5 mg, 34% yield) was synthesized according to synthetic procedure of step 8 of example 2.

MS (ESI) m/z: 1565.5 [M+H]+

### Example 11

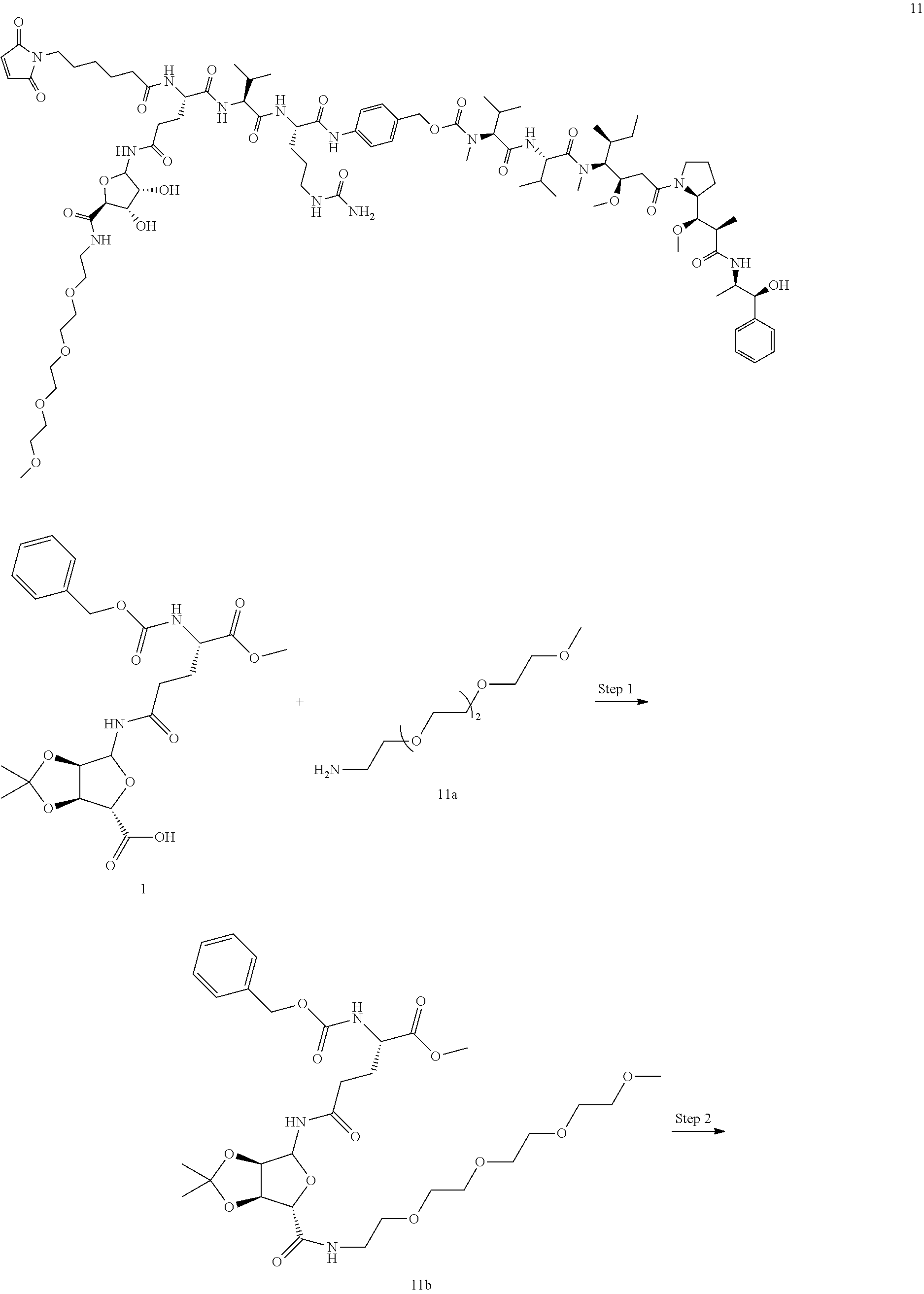

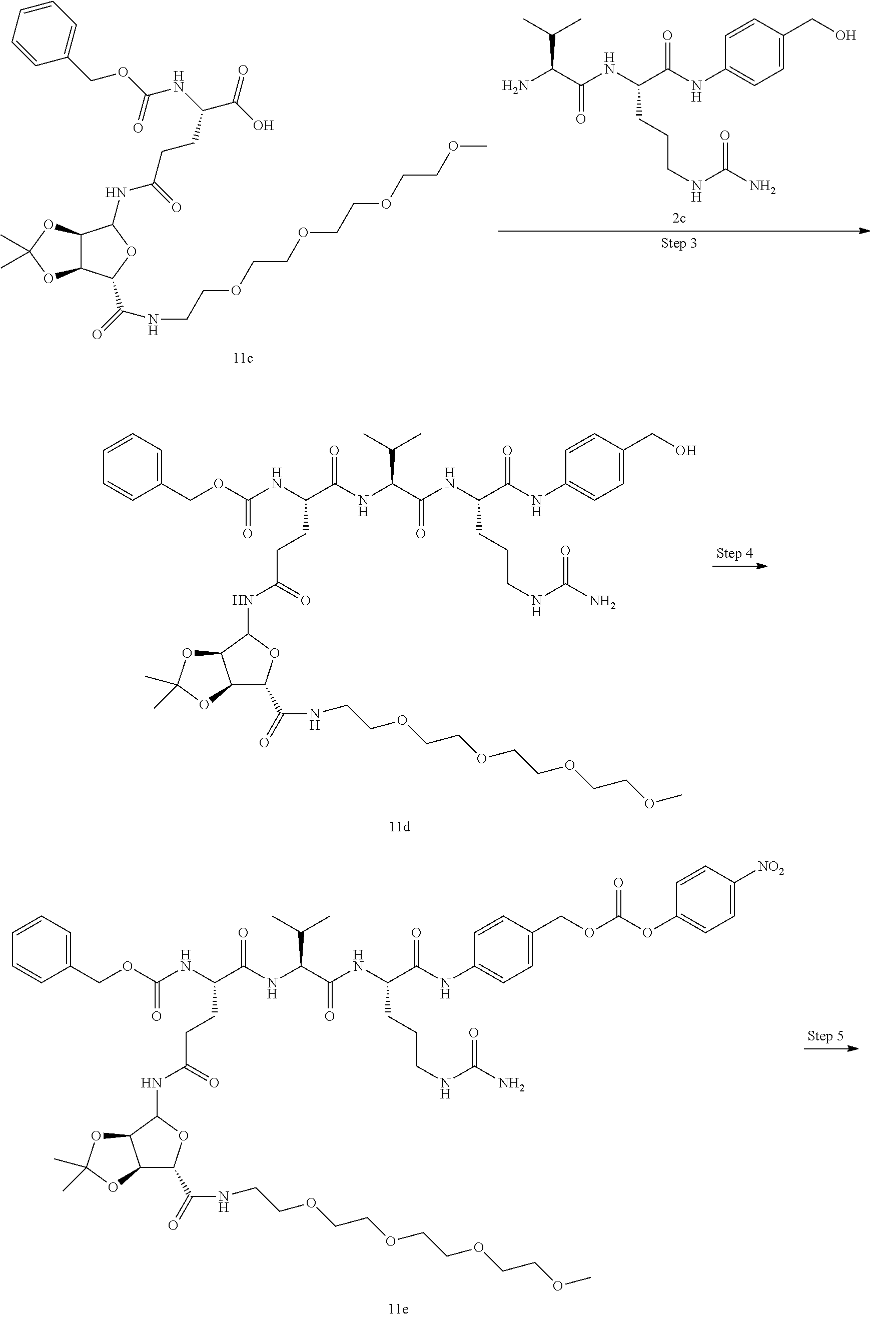

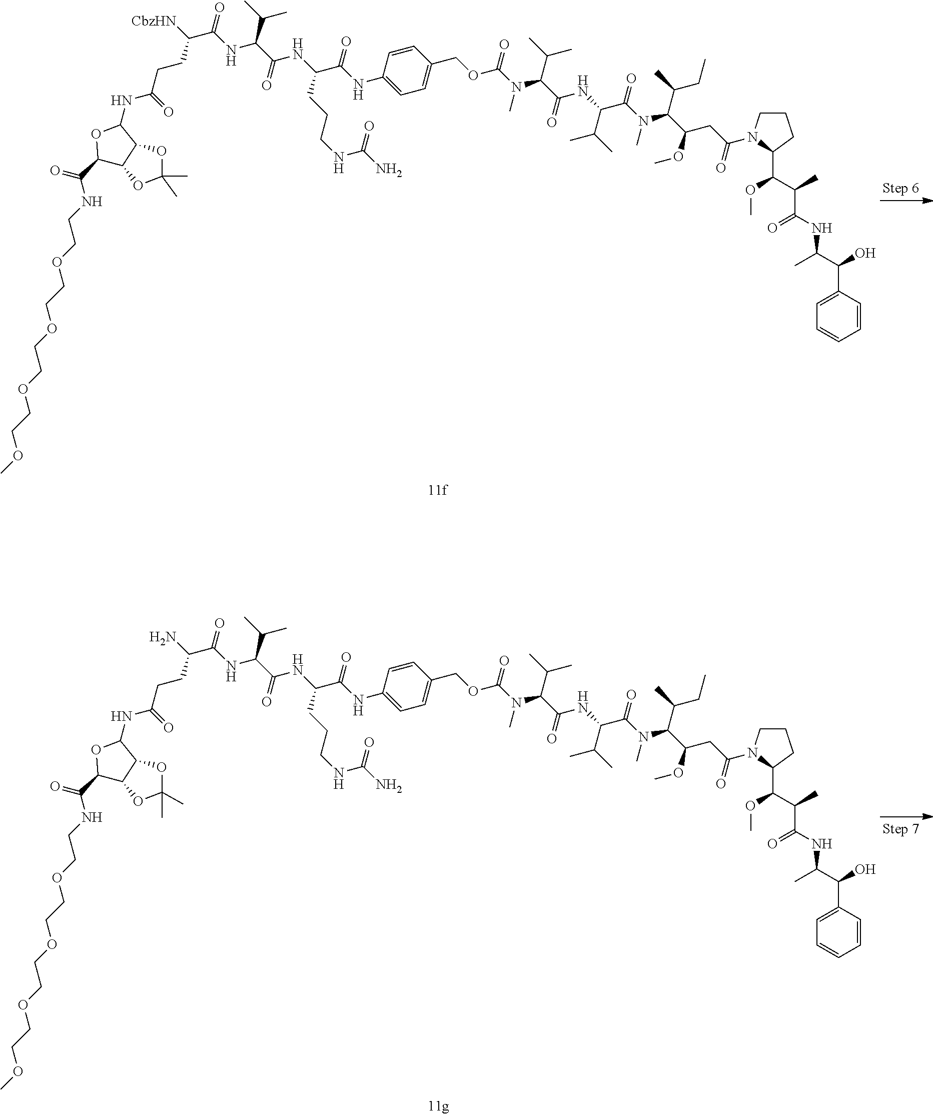

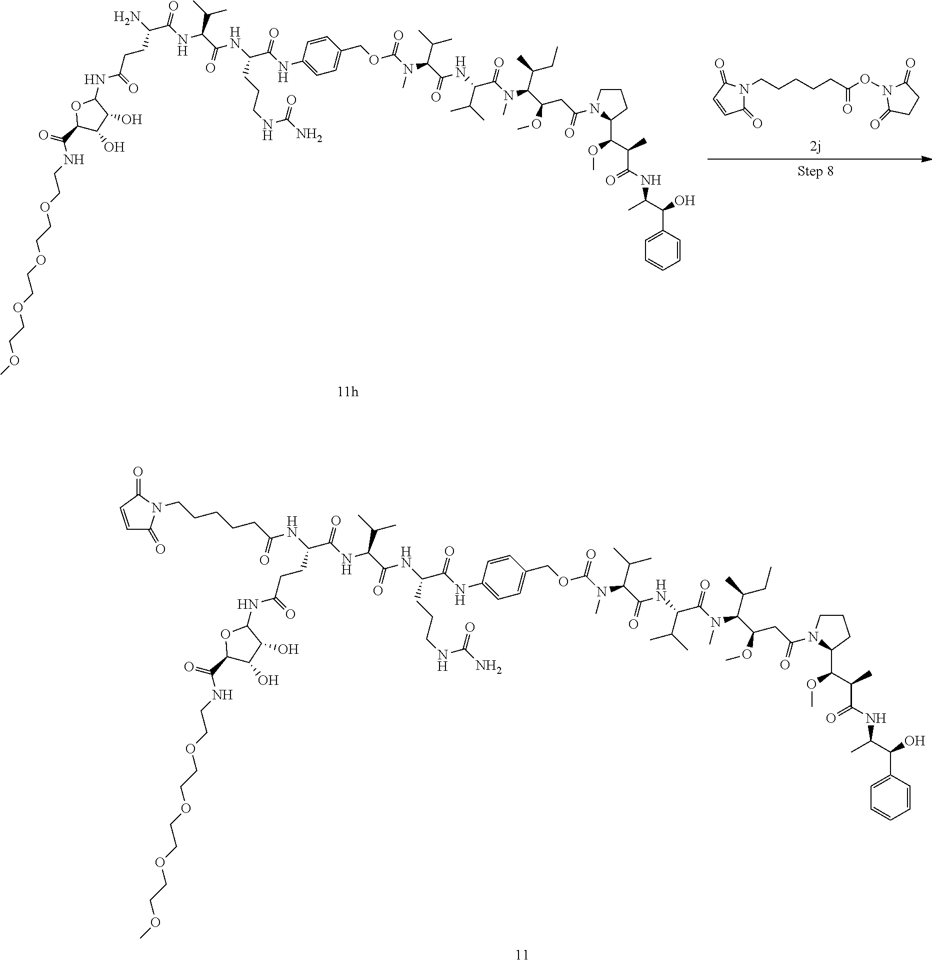

### Step 1

### Methyl N5-((3aR,6S,6aS)-6-((2,5,8,11-tetraoxatridecan-13-yl)carbamoyl)-2,2-dimethyltetrahydrofuro[3,4-d][1,3]dioxol-4-yl)-N2-((benzyloxy)carbonyl)-L-glutaminate (11b)

1 (300 mg, 0.62 mmol, synthesized according to the synthetic procedures of Example 1) was dissolved in DMF (4 mL) followed by the addition of HATU (309 mg, 0.81 mmol) and DIEA (242 mg, 1.87 mmol). The mixture was stirred at 25° C. for 15 min before addition of 11a (259 mg, 1.25 mmol). The resulting mixture was further stirred at 25° C. for 5 h. After complete reaction, the reaction mixture was purified by Prep HPLC (Method: column: XBridge Prep C18 OBD Sum 19*250 mm; Mobile phase: A—water (0.1% formic acid): B—acetonitrile; Flow rate: 20 mL/min) to afford 11b (164 mg, 45% yield).

MS (ESI) m/z: 670.6 [M+H]+

### Step 2

### N5-((3aR,6S,6aS)-6-((2,5,8,11-tetraoxatridecan-13-yl)carbamoyl)-2,2-dimethyltetrahydrofuro[3,4-d][1,3]dioxol-4-yl)-N2-((benzyloxy)carbonyl)-L-glutamine (11c)

11b (116 mg, 0.17 mmol) was dissolved in a mixed solvent of THE and MeOH (v:v=1:1, 0.6 mL) followed by the addition of LiOH·H2O (13 mg, 0.31 mmol) in H2O (0.3 mL). The resulting mixture was stirred at 40° C. for 1.5 h. After complete reaction, the reaction mixture was adjusted to pH=5 with aq. HCl (1 M) and purified by Prep HPLC (Method: column: XBridge Prep C18 OBD 5 μm 19*250 mm; Mobile phase: A—water (0.1% formic acid): B—acetonitrile; Flow rate: 20 mL/min) to afford 11c (62 mg, 55% yield).

MS (ESI) m/z: 656.5 [M+H]+ and 654.3 [M−H]−

### Step 3

### Benzyl ((S)-5-(((3aR,6S,6aS)-6-((2,5,8,11-tetraoxatridecan-13-yl)carbamoyl)-2,2-dimethyltetrahydrofuro[3,4-d][1,3]dioxol-4-yl)amino)-1-(((S)-1-(((S)-1-((4-(hydroxymethyl)phenyl)amino)-1-oxo-5-ureidopentan-2-yl)amino)-3-methyl-1-oxobutan-2-yl)amino)-1,5-dioxopentan-2-yl)carbamate (11d)

11c (58 mg, 0.09 mmol) was dissolved in DMF (2 mL) followed by the addition of HATU (44 mg, 0.11 mmol) and DIEA (34 mg, 0.27 mmol). The resulting mixture was stirred at 25° C. for 15 min before addition of 2c (Synthesized according to procedure revealed by patent “WO2020/14541A2”). The mixture was further stirred at 25° C. for 2 h. After complete reaction, the mixture was purified by Prep HPLC (Method: column: XBridge Prep C18 OBD Sum 19*250 mm; Mobile phase: A—water (0.1% formic acid): B—acetonitrile; Flow rate: 20 mL/min) to afford 11d (58 mg, 65% yield).

MS (ESI) m/z: 1017.8 [M+H]+

### Step 4

Benzyl ((S)-5-(((3aR,6S,6aS)-6-((2,5,8,11-tetraoxatridecan-13-yl)carbamoyl)-2,2-dimethyltetrahydrofuro[3,4-d][1,3]dioxol-4-yl)amino)-1-(((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)amino)-1,5-dioxopentan-2-yl)carbamate (11e)

11d (20 mg, 0.02 mmol) and para-nitrophenol carbonate (7 mg, 0.02 mmol) were dissolved in DMF (0.2 mL) followed by the addition of DIEA (8 mg, 0.06 mmol). The resulting mixture was stirred at 25° C. for 4 h. After complete reaction, H2O (1 mL) was added to quench the reaction. The reaction mixture was purified by Prep HPLC (Method: column: XBridge Prep C18 OBD Sum 19*250 mm; Mobile phase: A—water (0.1% formic acid): B—acetonitrile; Flow rate: 20 mL/min) to afford 11e (18 mg, 78% yield).

MS (ESI) m/z: 1017.8 [M+H]+

### Step 5

### 4-((5S,8S,11S)-5-(3-(((3aR,6S,6aS)-6-((2,5,8,11-tetraoxatridecan-13-yl)carbamoyl)-2,2-dimethyltetrahydrofuro[3,4-d][1,3]dioxol-4-yl)amino)-3-oxopropyl)-8-isopropyl-3,6,9-trioxo-1-phenyl-11-(3-ureidopropyl)-2-oxa-4,7,10-triazadodecan-12-amido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (11f)

11f (31 mg, 68% yield) was synthesized according to synthetic procedure of step 5 of example 2.

MS (ESI) m/z: 881.5 [M+2H]2+

### Step 6

### 4-((S)-2-((S)-2-((S)-5-(((3aR,6S,6aS)-6-((2,5,8,11-tetraoxatridecan-13-yl)carbamoyl)-2,2-dimethyltetrahydrofuro[3,4-d][1,3]dioxol-4-yl)amino)-2-amino-5-oxopentanamido)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (11 g)

11 g (25 mg, crude) was synthesized according to synthetic procedure of step 6 of example 2.

MS (ESI) m/z: 814.6 [M+2H]2+

### Step 7

### 4-((S)-2-((S)-2-((S)-5-(((3R,4S,5S)-5-((2,5,8,11-tetraoxatridecan-13-yl)carbamoyl)-3,4-dihydroxytetrahydrofuran-2-yl)amino)-2-amino-5-oxopentanamido)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (11 h)

11 h (6.5 mg, 27% yield) was synthesized according to synthetic procedure of step 7 of example 2.

MS (ESI) m/z: 1588.4 [M+H]+

### Step 8

### 4-((S)-2-((S)-2-((S)-5-(((3R,4S,5S)-5-((2,5,8,11-tetraoxatridecan-13-yl)carbamoyl)-3,4-dihydroxytetrahydrofuran-2-yl)amino)-2-(6-(2,5-dioxo-2,5-dihydro-1H-pyrrol-1-yl)hexanamido)-5-oxopentanamido)-3-methylbutanamido)-5-ureidopentanamido)benzyl ((S)-1-(((S)-1-(((3R,4S,5S)-1-((S)-2-((1R,2R)-3-(((1 S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (11)

11 (3.5 mg, 48% yield) was synthesized according to synthetic procedure of step 8 of example 2.

MS (ESI) m/z: 1781.0 [M+H]+

### Example 12

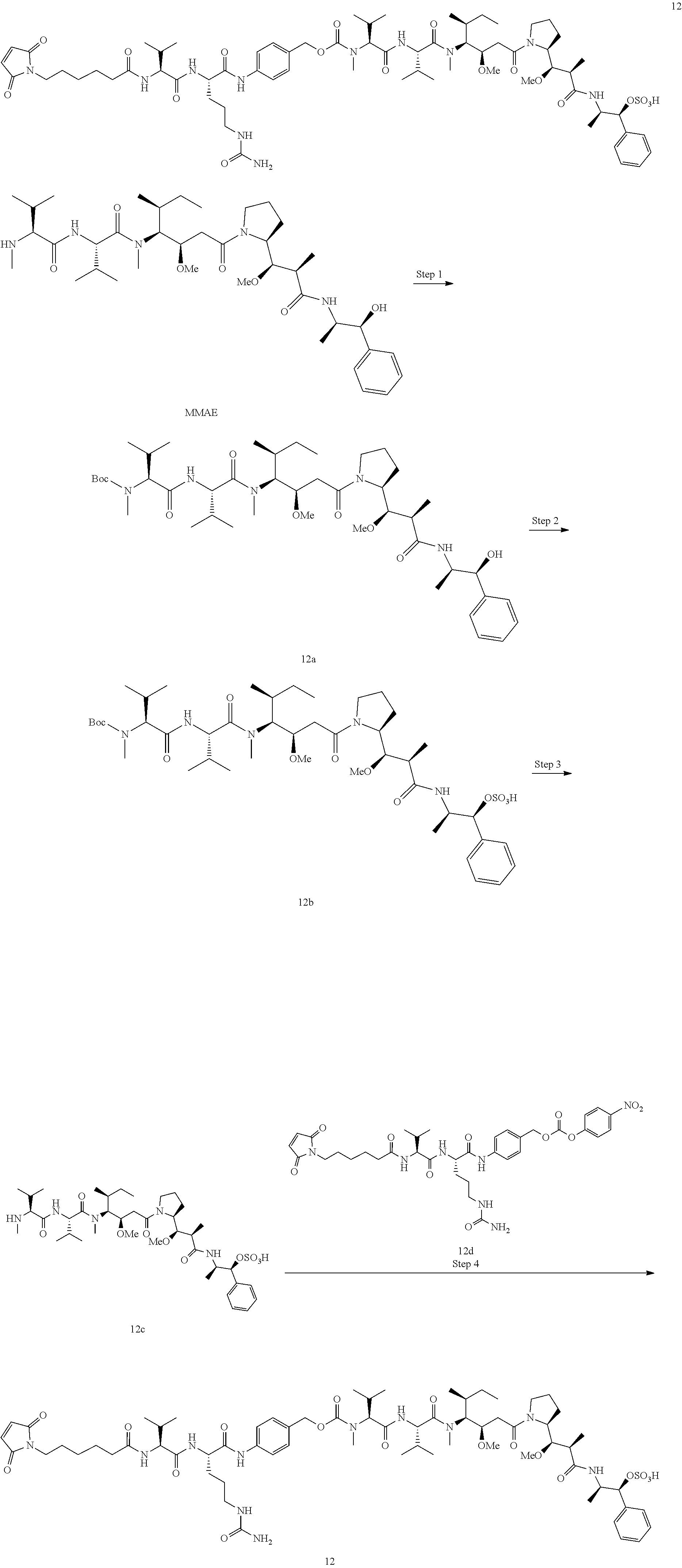

### Step 1

### Tert-butyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (12a)

Di-tert butyl decarbonate (60 μL, 0.15 mmol) was added into a EtOH solution (1 mL) of MMAE (100 mg, 0.14 mmol). The mixture was stirred overnight. After complete reaction, the reaction mixture was concentrated under reduced pressure and purified by flash column (eluent: DCM/MeOH, from 100/0 to 20/1 (v/v)) to afford 12a (115 mg, quant.).

MS (ESI) m/z: 818.8 [M+H]+

### Step 2

### (1S,2R)-2-((2R,3R)-3-((S)-1-((6S,9S,12S,13R)-12-((S)-sec-butyl)-6,9-diisopropyl-13-methoxy-2,2,5,11-tetramethyl-4,7,10-trioxo-3-oxa-5,8,11-triazapentadecan-15-oyl)pyrrolidin-2-yl)-3-methoxy-2-methylpropanamido)-1-phenylpropyl hydrogen sulfate (12b)

Et3N (48 μL, 0.34 mmol) and chlorosulfonic acid (14 μL, 0.22 mmol) were added into a DCM (1 mL) solution of 12a (70 mg, 0.08 mmol) at 0° C. The resulting mixture was stirred at this temperature for 1 h. After complete reaction, the reaction was quenched by addition of H2O (1 mL). The mixture was extracted with DCM (1 mL×4). The combined organic layers were dried over anhydrous Na2SO4, filtered and concentrated under reduced pressure to afford a brown oil as the crude product which was purified by flash column (eluent: DCM/MeOH, from 100/0 to 10/1 (v/v)) to afford 12b as a white solid (47 mg, 65% yield).

MS (ESI) m/z: 896.6 [M−H]−

### Step 3

### (1S,2R)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-1-phenylpropyl hydrogen sulfate (12c)

12b (40 mg, 0.04 mmol) was dissolved in DCM (0.8 mL) followed by the addition of TFA (0.2 mL). The resulting mixture was stirred at 0° C. for 2 h. After complete reaction, the mixture was concentrated under reduced pressure to afford crude 12c (50 mg) which was used directly in next step.

MS (ESI) m/z: 796.7 [M−H]−

### Step 4

### (1S,2R)-2-((2R,3R)-3-((S)-1-((5S,8S,11S,12R)-11-((S)-sec-butyl)-1-(4-((S)-2-((S)-2-(6-(2,5-dioxo-2,5-dihydro-1H-pyrrol-1-yl)hexanamido)-3-methylbutanamido)-5-ureidopentanamido)phenyl)-5,8-diisopropyl-12-methoxy-4,10-dimethyl-3,6,9-trioxo-2-oxa-4,7,10-triazatetradecan-14-oyl)pyrrolidin-2-yl)-3-methoxy-2-methylpropanamido)-1-phenylpropyl hydrogen sulfate (12)

12c (27 mg, 0.03 mmol) and 12d (37 mg, 0.05 mmol, commercially available) were dissolved in DMF (0.5 mL) followed by the addition of HOBt (0.9 mg, 0.01 mmol) and DIEA (56 μL, 0.34 mmol). The mixture was stirred at 25° C. overnight. After complete reaction, the mixture was purified by Prep HPLC (Method: column: XBridge Prep C18 OBD Sum 19*250 mm; Mobile phase: A—water (0.1% formic acid): B—acetonitrile; Flow rate: 20 mL/min) to afford 12 as a white solid (5.4 mg, 13% yield)

MS (ESI) m/z: 1395.0 [M−H]−

### Example 13

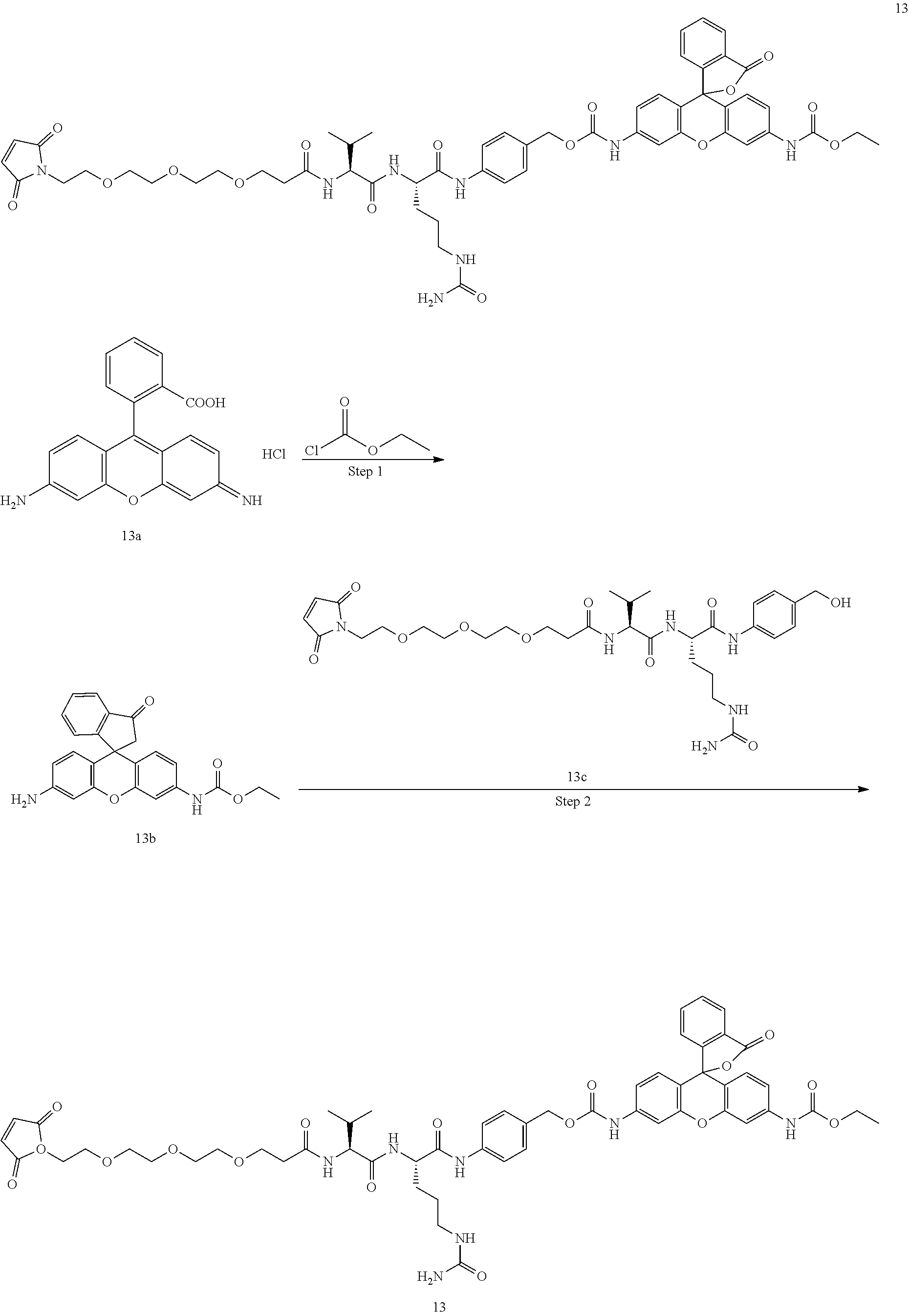

### Step 1

### Ethyl (3′-amino-3-oxo-3H-spiro[isobenzofuran-1,9′-xanthen]-6′-yl)carbamate (13b)

To the solution of 2-(6-amino-3-imino-3H-xanthen-9-yl)benzoic acid hydrochloride 13a (800 mg, 2.18 mmol) and DIEA (705 mg, 5.4 mmol) in DMF (5 mL) was added dropwise ethyl chloroformate (284 mg, 2.6 mmol) at 0° C. The solution was stirred at r.t. for 15 h after the addition was completed. Ice water (5 mL) was added to the reaction solution to quench the reaction. The solution was extracted with EtOAc (20 mL*3), washed with water (10 mL*2) and brine (10 mL), dried over anhydrous sodium sulfate, concentrated and purified by flash column chromatography (Petroleum ether/EtOAc=100/0 to 70/30) to give the title compound 13b (321 mg, 36.3% yield).

MS (ESI) m/z: 403.0 [M+H]+

### Step 2

### 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl ethyl (3-oxo-3H-spiro[isobenzofuran-1,9′-xanthene]-3′,6′-diyl)dicarbamate (13)

To the solution of 13b (90 mg, 0.22 mmol) and 2,6-lutidine (72 mg, 0.67 mmol) in DCM (15 mL) was added triphosgene (33 mg, 0.11 mmol) in portions under nitrogen atmosphere at 0° C., stirred at r.t. for 3 h. The solution of the intermediate 13c (148 mg, 0.22 mmol) in DCM (3 mL) was added dropwise at 0° C., stirred at r.t. for 15 h. Water (5 mL) was added to quench the reaction. The organic phase was dried over anhydrous sodium sulfate, concentrated and purified by flash column chromatography to give the title compound 13 (30 mg, 12.5% yield).

MS (ESI) m/z: 1091.4 [M+H]+

### Example 14

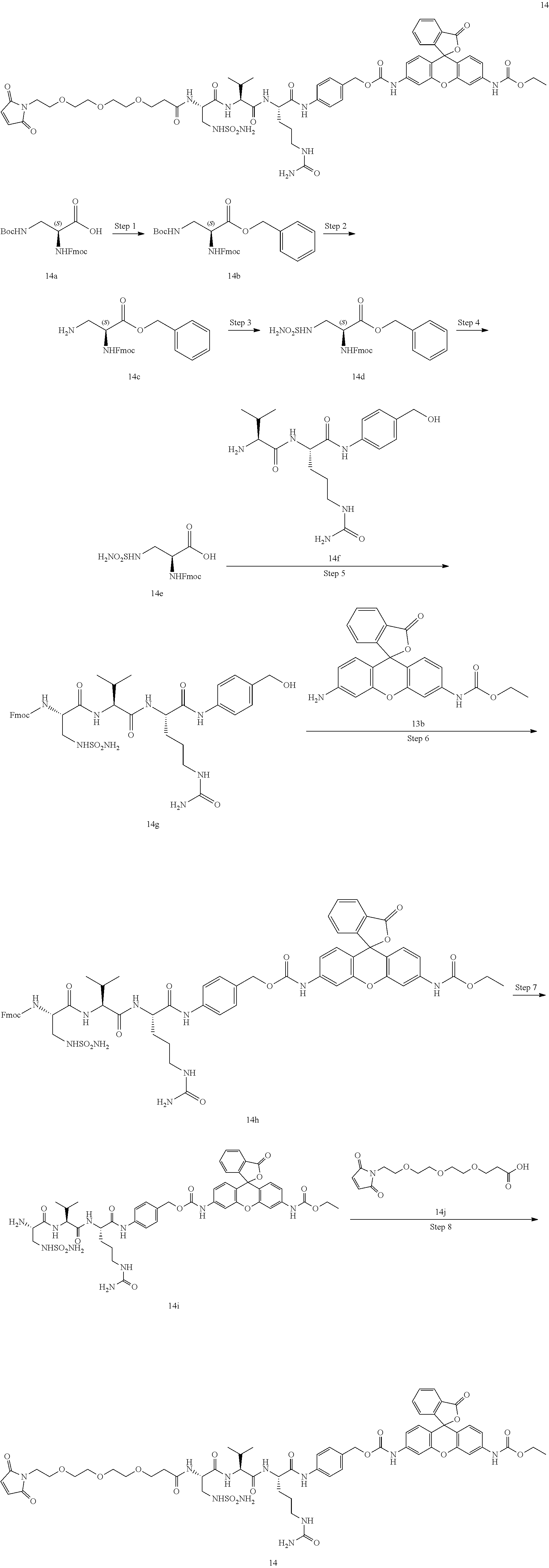

### Step 1

### Benzyl (S)-2-((((9H-fluoren-9-yl)methoxy)carbonyl)amino)-3-((tert-butoxycarbonyl)amino)propanoate (14b)

(S)-2-((((9H-fluoren-9-yl)methoxy)carbonyl)amino)-3-((tert-butoxycarbonyl)amino)propanoic acid 14a (10.0 g, 23.45 mmol) and benzyl bromide (16.0 g, 93.79 mmol) were dissolved in DMF (118 mL). To the solution was added sodium bicarbonate (3.94 g, 46.90 mmol), stirred at r.t. for 15 h. EtOAc (1 L) was added, washed with water (200 mL*3) and brine (200 mL). The organic phase was dried over anhydrous sodium sulfate, concentrated under vacuum and purified by flash column chromatography to give the title compound 14b (7 g, 57.8% yield).

MS (ESI) m/z: 517.3 [M+H]+

### Step 2

### Benzyl (S)-2-((((9H-fluoren-9-yl)methoxy)carbonyl)amino)-3-aminopropanoate (14c)

To the solution of 14b (7.0 g, 13.55 mmol) in DCM (20 mL) was added TFA (10 mL), stirred at r.t. for 2 h. The solution was concentrated and the pH of the solution was adjusted to 8 with saturated sodium bicarbonate, extracted with EtOAc (200 mL*3), dried over anhydrous sodium sulfate, concentrated under vacuum and purified by flash column chromatography to give the title compound 14c (4.5 g, 79.7% yield).

MS (ESI) m/z: 417.2 [M+H]+

### Step 3

### Benzyl (S)-2-((((9H-fluoren-9-yl)methoxy)carbonyl)amino)-3-(sulfamoylamino)propanoate (14d)

To the solution of 14c (4.0 g, 9.60 mmol) and TEA (1.5 g, 14.41 mmol) in DCM (100 mL) was added chlorosulfonamide (1.2 g, 10.57 mmol) in portions at 0° C., stirred at r.t. for 15 h. Water (100 mL) was added to quench the reaction, extracted with EtOAc (200 mL*3), dried over anhydrous sodium sulfate, concentrated under vacuum and purified by flash column chromatography to give the title compound 14d (1.2 g, 25.2% yield).

MS (ESI) m/z: 496.2 [M+H]+

### Step 4

### (S)-2-((((9H-fluoren-9-yl)methoxy)carbonyl)amino)-3-(sulfamoylamino)propanoic acid (14e)

To the solution of 14d (1.1 g, 2.22 mmol) in MeOH (20 mL) and THF (20 mL) was added wet Pd/C (10%, 1.1 g), stirred under H2 (15 psi) atmosphere at r.t. for 1 h. The solution was filtered through Celite, concentrated under vacuum to give the title compound 14e (600 mg, 66.7% yield).

MS (ESI) m/z: 406.1 [M+H]+

### Step 5

### (9H-fluoren-9-yl)methyl ((S)-1-(((S)-1-(((S)-1-((4-(hydroxymethyl)phenyl)amino)-1-oxo-5-ureidopentan-2-yl)amino)-3-methyl-1-oxobutan-2-yl)amino)-1-oxo-3-(sulfamoylamino)propan-2-yl)carbamate (14 g)

To the solution of 14e (600 mg, 1.48 mmol) and 14f (562 mg, 1.48 mmol) in DMF (10 mL) was added DIEA (383 mg, 2.96 mmol). HATU (732 mg, 1.92 mmol) was added to the solution at 0° C., stirred at r.t. for 3 h. The reaction solution was purified by flash column chromatography to give the title compound 14g (600 mg, 52.9% yield).

MS (ESI) m/z: 767.3 [M+H]+

### Step 6

### 4-((5S,8S,11S)-1-(9H-fluoren-9-yl)-8-isopropyl-3,6,9-trioxo-5-((sulfamoylamino)methyl)-11-(3-ureidopropyl)-2-oxa-4,7,10-triazadodecan-12-amido)benzyl ethyl (3-oxo-3H-spiro[isobenzofuran-1,9′-xanthene]-3′,6′-diyl)dicarbamate (14 h)

14 h (50 mg, 8.0% yield) was synthesized according to the procedure described in Step 2 of Example 13.

MS (ESI) m/z: 1195.4 [M+H]+

### Step 7

### 4-((S)-2-((S)-2-((S)-2-amino-3-(sulfamoylamino)propanamido)-3-methylbutanamido)-5-ureidopentanamido)benzyl ethyl (3-oxo-3H-spiro[isobenzofuran-1,9′-xanthene]-3′,6′-diyl)dicarbamate (14i)

To the solution of 14 h (50 mg, 0.04 mmol) in DMF (3 mL) was added DBU (26 mg, 0.17 mmol), stirred at r.t. for 2 h. The reaction solution was purified by flash column chromatography to give the title compound 14i (20 mg, 49.1% yield).

MS (ESI) m/z: 973.4 [M+H]+

### Step 8

### 4-((2S,5S,8S)-21-(2,5-dioxo-2,5-dihydro-1H-pyrrol-1-yl)-5-isopropyl-4,7,10-trioxo-8-((sulfamoylamino)methyl)-2-(3-ureidopropyl)-13,16,19-trioxa-3,6,9-triazahenicosanamido)benzyl ethyl (3-oxo-3H-spiro[isobenzofuran-1,9′-xanthene]-3′,6′-diyl)dicarbamate (14)

To the solution of 14i (20 mg, 0.02 mmol), 14j (7 mg, 1.40 mmol) and DIEA (5.3 mg, 0.04 mmol) in DMF (3 mL) was added HATU (10 mg, 0.03 mmol) at 0° C., stirred at r.t. for 3 h. The reaction solution was purified by prep-HPLC to give the title compound 14 (8.3 mg, 32.1% yield).

MS (ESI) m/z: 1256.3 [M+H]+

### Example 15

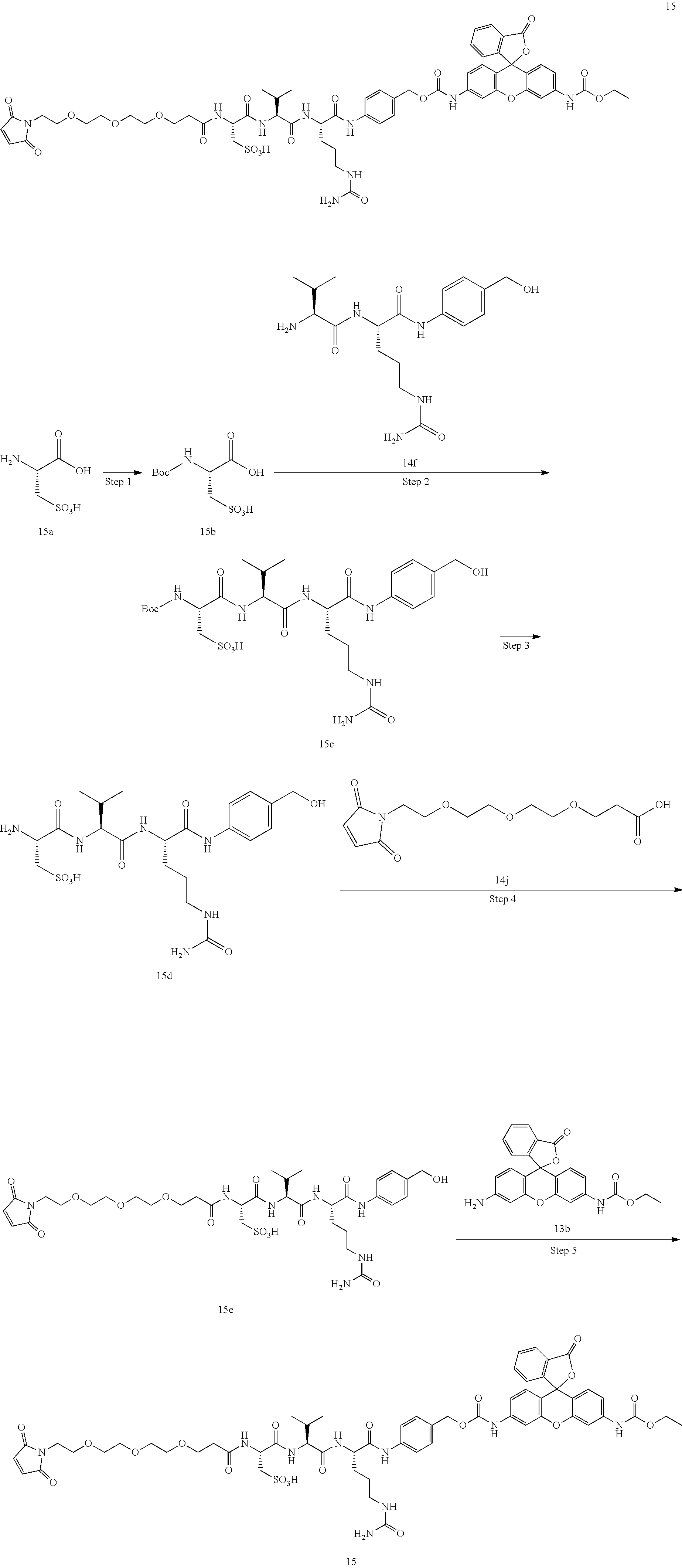

### Step 1

### (tert-butoxycarbonyl)(sulfo)-D-alanine (15b)

To the solution of sulfo-D-alanine 15a (1.2 g, 7.1 mmol) and di-tert-butyl dicarbonate (1.9 g, 8.52 mmol) in DMF (10 mL) was added DIEA (1.4 g, 14.2 mmol), stirred at r.t. overnight. The reaction solution was concentrated under vacuum to give the title compound 15b (2.0 g, crude) as a colorless oil.

MS (ESI) m/z: 267.7 [M−H]−

### Step 2

### (R)-2-((tert-butoxycarbonyl)amino)-3-(((S)-1-(((S)-1-((4-(hydroxymethyl)phenyl)amino)-1-oxo-5-ureidopentan-2-yl)amino)-3-methyl-1-oxobutan-2-yl)amino)-3-oxopropane-1-sulfonic acid (15c)

To the solution of 15b (100 mg, 0.37 mmol) and 14f (99 mg, 0.26 mmol) in DMF (3 mL) was added DIEA (1.5 g, 11.9 mmol) and BOP (246 mg, 0.56 mmol), stirred in an ice bath for 0.5 h. The reaction solution was purified by prep-HPLC (water/MeCN=100/0 to 80/20, 30 min; detection wavelength: 254/220 nm) to give the title compound 15c (180 mg, 77.1% yield) as a pale yellow solid.

MS (ESI) m/z: 629.1 [M−H]−

### Step 3

### (R)-2-amino-3-(((S)-1-(((S)-1-((4-(hydroxymethyl)phenyl)amino)-1-oxo-5-ureidopentan-2-yl)amino)-3-methyl-1-oxobutan-2-yl)amino)-3-oxopropane-1-sulfonic acid (15d)

To the solution of 15c (210 mg, 0.33 mmol) in DCM (3 mL) was added TFA (1 mL), stirred at r.t. for 1 h, concentrated. The residue was dissolved in 1,4-dioxane (2 mL), the solution of NaOH (67 mg, 1.67 mmol) in water (1 mL) was added, stirred at r.t. for 1 h. The pH of the reaction solution was adjusted to 6 with 1N HCl solution. The crude product was purified by prep-HPLC (water/MeCN=100/0 to 60/40, 30 min; detection wavelength: 254/220 nm) to give the title compound 15d (160 mg, 91.4% yield) as a light yellow solid.

MS (ESI) m/z: 531.2 [M+H]+

### Step 4

### (R)-1-(2,5-dioxo-2,5-dihydro-1H-pyrrol-1-yl)-14-(((S)-1-(((S)-1-((4-(hydroxymethyl)phenyl)amino)-1-oxo-5-ureidopentan-2-yl)amino)-3-methyl-1-oxobutan-2-yl)carbamoyl)-12-oxo-3,6,9-trioxa-13-azapentadecane-15-sulfonic acid (15e)

To the solution of 15d (180 mg, 0.34 mmol) and 14j (153 mg, 0.51 mmol) in DMF (5 mL) was added DIEA (88 mg, 0.68 mmol) and BOP (225 mg, 0.51 mmol) in an ice bath, stirred at 0° C. for 0.5 h. The reaction solution was purified by prep-HPLC (water/MeCN=100/0 to 80/20, 30 min; detection wavelength: 254/220 nm) to give the title compound 15e (130 mg, 47.0% yield) as a pale yellow solid.

MS (ESI) m/z: 814.2 [M+H]+

### Step 5

### (14R)-1-(2,5-dioxo-2,5-dihydro-1H-pyrrol-1-yl)-14-(((2S)-1-(((2S)-1-((4-((((3′-((ethoxycarbonyl)amino)-3-oxo-3H-spiro[isobenzofuran-1,9′-xanthen]-6′-yl)carbamoyl)oxy)methyl)phenyl)amino)-1-oxo-5-ureidopentan-2-yl)amino)-3-methyl-1-oxobutan-2-yl)carbamoyl)-12-oxo-3,6,9-trioxa-13-azapentadecane-15-sulfonic acid (15)

To the solution of 13b (64 mg, 0.16 mmol) and 2,6-lutidine (51 mg, 0.48 mmol) in DCM (10 mL) was added triphosgene (24 mg, 0.08 mmol) in portions in an ice bath, stirred at r.t. for 3 h. To this was added the solution of 15e (130 mg, 0.16 mmol) in DCM (2 mL), stirred at r.t. overnight. Ice water (0.2 mL) was added to quench the reaction. The reaction solution was concentrated. The residue was purified by flash column chromatography (DCM/MeOH=6/1). The crude product was purified by prep-HPLC (Column: XBridge Prep OBD C18 Column, 30*150 mm; water (0.05% TFA)/MeCN=62/38 to 42/58, 7 min; detection wavelength: 254/220 nm) to give the title compound 15 (6.0 mg, 3.0% yield) as an orange solid.

MS (ESI) m/z: 1242.3 [M+H]+

### Example 16

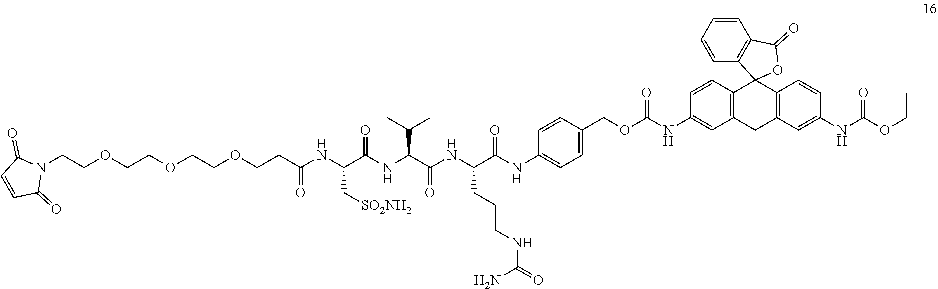 [SMILES: CCOC(=O)Nc1ccc2c(c1)Cc1cc(NC(=O)OCc3ccc(NC(=O)[C@H](CCCNC(N)=O)NC(=O)[C@@H](NC(=O)[C@H](CS(N)(=O)=O)NC(=O)CCOCCOCCOCCN4C(=O)C=CC4=O)C(C)C)cc3)ccc1C21OC(=O)c2ccccc21]

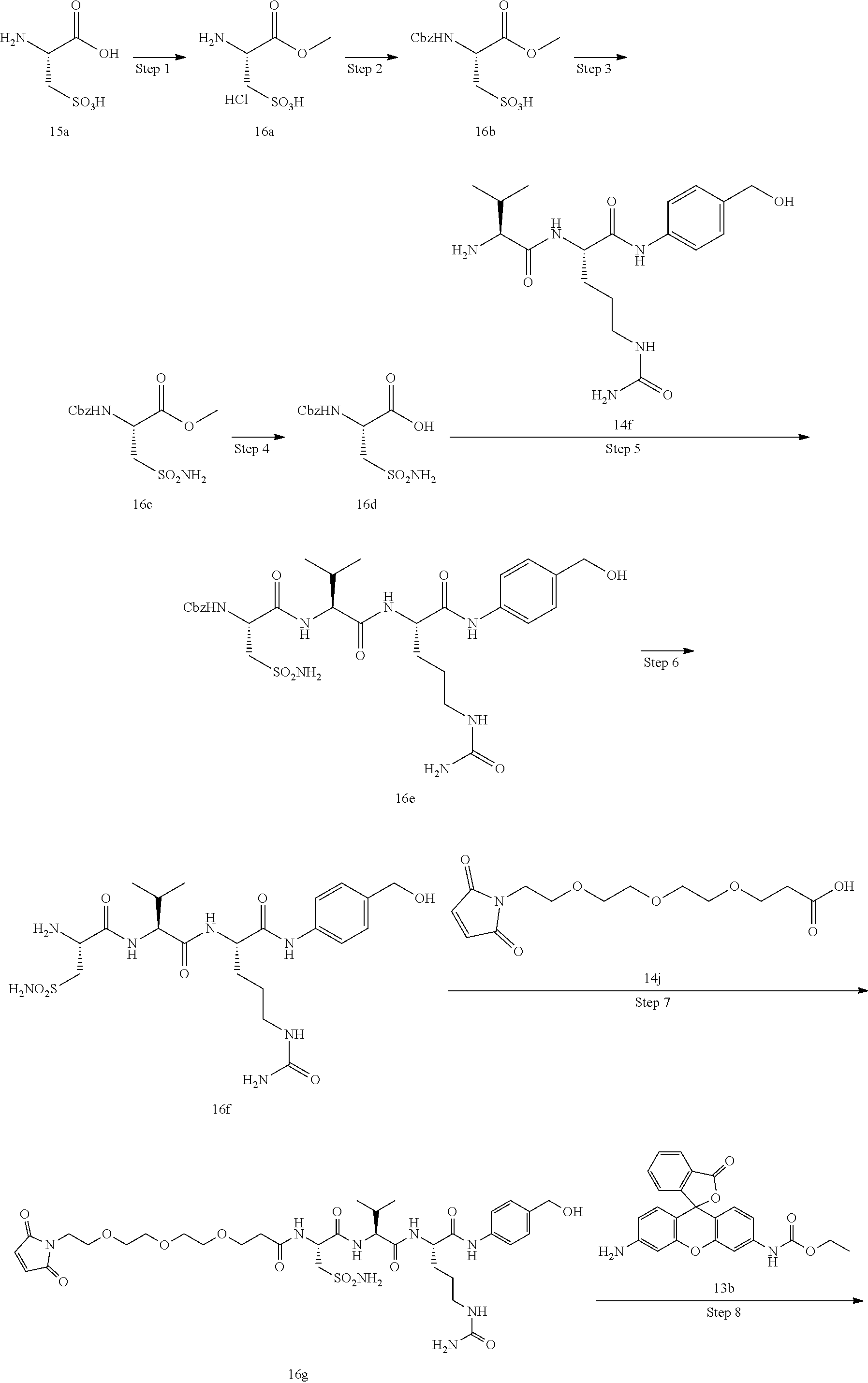

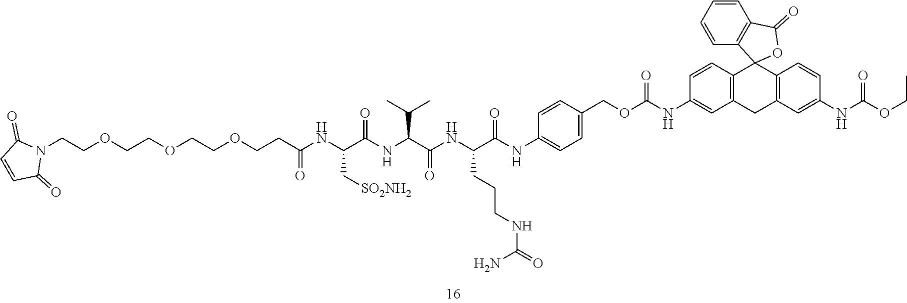 [SMILES: CCOC(=O)Nc1ccc2c(c1)Cc1cc(NC(=O)OCc3ccc(NC(=O)[C@H](CCCNC(N)=O)NC(=O)[C@@H](NC(=O)[C@H](CS(N)(=O)=O)NC(=O)CCOCCOCCOCCN4C(=O)C=CC4=O)C(C)C)cc3)ccc1C21OC(=O)c2ccccc21]

### Step 1

### (R)-2-amino-3-methoxy-3-oxopropane-1-sulfonic acid hydrochloride (16a)

To the solution of sulfo-D-alanine 15a (15 g, 88.7 mmol) in MeOH (500 mL) was added dropwise thionyl chloride (211 g, 1.77 mol) at 0° C. within 30 min. The reaction solution was stirred at r.t. overnight. The solution was concentrated under vacuum to give the title compound 16a (18 g, crude) as a white solid.

MS (ESI) m/z: 184.0 [M−HCl+H]+

### Step 2

### (R)-2-(((benzyloxy)carbonyl)amino)-3-methoxy-3-oxopropane-1-sulfonic acid (16b)

To the solution of 16a (18 g, 60.9 mmol) in THE (75 mL) was added the solution of sodium bicarbonate (12.8 g, 152.2 mmol) in water (25 mL). Benzyl chloroformate (12.5 g, 73.0 mmol) was added dropwise at 0° C., stirred at r.t. overnight. The reaction solution was extracted with EtOAc (300 mL*3), the organic phase was washed with brine (200 mL*3), dried over anhydrous sodium sulfate, filtered and concentrated under vacuum. The residue was purified by flash column chromatography (Petroleum Ether/Ethyl Acetate=1/1) to give the title compound 16b (15 g, 77.6% yield) as a white solid.

MS (ESI) m/z: 335.2 [M+NH3]+

### Step 3

### Methyl ((benzyloxy)carbonyl)(sulfamoyl)-D-alaninate (16c)

To the solution of 16b (14.0 g, 44.12 mmol) and triphenylphosphine (17.4 g, 66.2 mmol) in DCM (500 mL) was added dropwise thionyl chloride (8.9 g, 75.0 mmol) in an ice bath, stirred at r.t. for 3 h. The reaction solution was concentrated. The residue was dissolved in 0.5 M solution of ammonia in THE (150 mL), stirred at r.t. overnight. The reaction solution was concentrated. The residue was dissolved in MeOH (20 mL), filtered. The filtration was purified by prep-HPLC (Column: XBridge Prep OBD C18 Column, 30*150 mm; water (0.5% ammonium bicarbonate)/acetonitrile=100/0 to 70/30, 60 min; detection wavelength: 254/220 nm) to give the title compound 16c (6.0 g, 43.0% yield) as a white solid.

MS (ESI) m/z: 318.0 [M+H]+

### Step 4

### ((benzyloxy)carbonyl)(sulfamoyl)-D-alanine (16d)

The intermediate 16c (6.0 g, 19.0 mmol) and trimethyltin hydroxide (13.7 g, 75.9 mmol) were dissolved in 1,2-dichloroethane (50 mL), stirred at 80° C. for 3 h. The reaction solution was filtered and purified by prep-HPLC (Column: XBridge Prep OBD C18 Column, 30*150 mm; water (0.5% ammonium bicarbonate)/acetonitrile=100/0 to 80/20, 60 min; detection wavelength: 254/220 nm) to give the title compound 16d (1.8 g, 31.3% yield) as a pale yellow solid.

MS (ESI) m/z: 325.0 [M+Na]+

### Step 5

### Benzyl ((R)-1-(((S)-1-(((S)-1-((4-(hydroxymethyl)phenyl)amino)-1-oxo-5-ureidopentan-2-yl)amino)-3-methyl-1-oxobutan-2-yl)amino)-1-oxo-3-sulfamoylpropan-2-yl)carbamate (16e)

To the solution of 16d (1.8 g, 5.9 mmol) and 14f (2.3 g, 5.9 mmol) in DMF (20 mL) was added DIEA (1.5 g, 11.9 mmol) and HATU (2.7 g, 7.2 mmol), stirred at r.t. for 3 h. The reaction solution was purified by prep-HPLC (Column: XBridge Prep OBD C18 Column, 30*150 mm; water (0.5% ammonium bicarbonate)/acetonitrile=100/0 to 20/80, 60 min; detection wavelength: 254/220 nm) to give the title compound 16e (500 mg, 12.8% yield) as a pale yellow solid.

MS (ESI) m/z: 664.3 [M+H]+

### Step 6

### (S)-2-((S)-2-((R)-2-amino-3-sulfamoylpropanamido)-3-methylbutanamido)-N-(4-(hydroxymethyl)phenyl)-5-ureidopentanamide (16f)

The intermediate 16e (400.0 mg, 0.6 mmol) was dissolved in TFA (5 mL), stirred at 50° C. for 6 h, concentrated. The residue was dissolved in 1,4-dioxane (5 mL), to this was added the solution of lithium hydroxide (72.2 mg, 3.0 mmol) in water (2.5 mL), stirred at r.t. for 1 h. The reaction was concentrated and purified by prep-HPLC (Column: XBridge Prep OBD C18 Column, 30*150 mm; water (10M ammonium bicarbonate)/acetonitrile=80/20 to 50/50, 8 min; detection wavelength: 254/220 nm) to give the title compound 16f (150 mg, 47.2% yield) as a white solid.

MS (ESI) m/z: 530.3 [M+H]+

### Step 7

### (S)-2-((14R,17S)-1-(2,5-dioxo-2,5-dihydro-1H-pyrrol-1-yl)-17-isopropyl-12,15-dioxo-14-(sulfamoylmethyl)-3,6,9-trioxa-13,16-diazaoctadecan-18-amido)-N-(4-(hydroxymethyl)phenyl)-5-ureidopentanamide (16 g)

To the solution of 16f (150 mg, 0.28 mmol) and 14j (93.9 mg, 0.31 mmol) in DMF (5 mL) was added DIEA (73.2 mg, 0.57 mmol) and HATU (129.2 mg, 0.34 mmol), stirred at r.t. for 2 h. The reaction solution was purified by prep-HPLC (Column: Xselect CSH OBD Column 30*150 mm, 5 m; water (0.1% FA)/acetonitrile=95/5 to 68/32, 10 min; detection wavelength: 254/220 nm) to give the title compound 16g (110 mg, 48.3% yield) as a white solid.

MS (ESI) m/z: 813.4 [M+H]+

### Step 8

### 4-((2S,5S,8R)-21-(2,5-dioxo-2,5-dihydro-1H-pyrrol-1-yl)-5-isopropyl-4,7,10-trioxo-8-(sulfamoylmethyl)-2-(3-ureidopropyl)-13,16,19-trioxa-3,6,9-triazahenicosanamido)benzyl ethyl (3-oxo-3H-spiro[isobenzofuran-1,9′-xanthene]-3′,6′-diyl)dicarbamate (16)

To the solution of 13b (65.3 mg, 0.16 mmol) and 2,6-lutidine (52.2 mg, 0.49 mmol) in DCM (20 mL) was added triphosgene (24.1 mg, 0.08 mmol) in portions in an ice bath, stirred at r.t. for 4 h. To the solution was added 16 g (110.0 mg, 0.14 mmol), stirred at r.r. overnight. Water (0.2 mL) was added to quench the reaction, concentrated. The residue was purified by flash column chromatography (DCM/MeOH=5/1). The crude product was purified by prep-HPLC (Column: XBridge Prep OBD C18 Column, 19*250 mm, 5 m; water (0.1% FA)/acetonitrile=80/20 to 50/50, 8 min; detection wavelength: 254/220 nm) to give the title compound 16 (5.9 mg, 3.4% yield) as a pale pink solid.

MS (ESI) m/z: 1241.3 [M+H]+

### Example 17

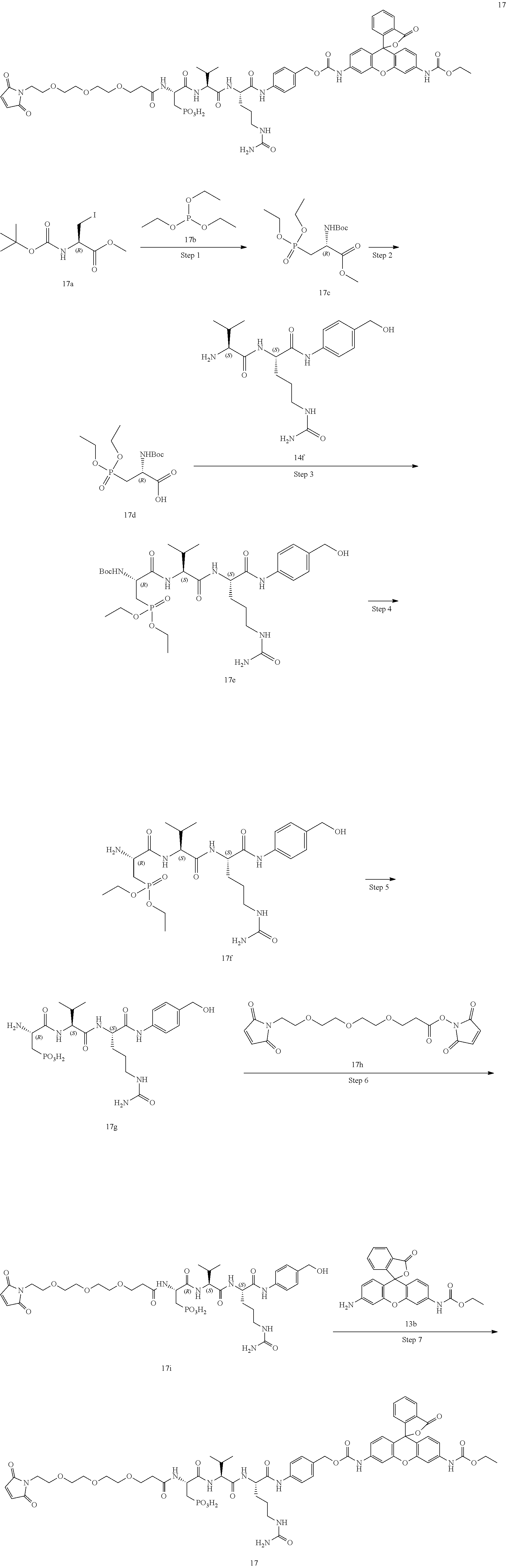

### Step 1

### Methyl (R)-2-((tert-butoxycarbonyl)amino)-3-(diethoxyphosphoryl)propanoate (17c)

Methyl (R)-2-((tert-butoxycarbonyl)amino)-3-iodopropanoate 17a (10 g, 30.38 mmol, commercial available) was mixed with triethyl phosphite (100 mL, 601.84 mmol), stirred at 140° C. overnight. The reaction solution was concentrated and purified by flash column chromatography (Petroleum ether/ethyl acetate, gradient elution) to give the title compound 17c (7.5 g, 72.8% yield).

MS (ESI) m/z: 340.3 [M+H]+

### Step 2

### (R)-2-((tert-butoxycarbonyl)amino)-3-(diethoxyphosphoryl)propanoic acid (17d)

To the solution of 17c (7.5 g, 22.1 mmol) in THE (40 mL) and water (20 mL) was added lithium hydroxide monohydrate (2.1 g, 88.53 mmol), stirred at r.t. overnight. The pH of the reaction solution was adjusted to 6 with 1M HCl, concentrated and purified by reverse phase column chromatography (Column: C18; water (0.1% FA)/acetonitrile, gradient elution, flow rate: 20 mL/min). The desired fraction was lyophilized to give the title compound 17d (2.8 g, 38.9% yield).

MS (ESI) m/z: 326.1 [M+H]+

### Step 3

### Tert-butyl ((R)-3-(diethoxyphosphoryl)-1-(((S)-1-(((S)-1-((4-(hydroxymethyl)phenyl)amino)-1-oxo-5-ureidopentan-2-yl)amino)-3-methyl-1-oxobutan-2-yl)amino)-1-oxopropan-2-yl)carbamate (17e)

To the solution of 17d (2.8 g, 8.61 mmol) in DMF (40 mL) were added HATU (3.9 g, 10.33 mmol) and DIEA (2.2 g, 17.22 mmol), stirred at r.t. for 10 min. To the solution was added 14f (3.6 g, 9.47 mmol, commercial available), stirred at r.t. for 2 h. The reaction solution was purified by reverse phase column chromatography (Column: C18; water (0.5% ammonium bicarbonate)/acetonitrile, gradient elution, flow rate: 20 mL/min). The desired fraction was lyophilized to give the title compound 17e (4.5 g, 76.1% yield).

MS (ESI) m/z: 686.3 [M+H]+

### Step 4

### Diethyl ((R)-2-amino-3-(((S)-1-(((S)-1-((4-(hydroxymethyl)phenyl)amino)-1-oxo-5-ureidopentan-2-yl)amino)-3-methyl-1-oxobutan-2-yl)amino)-3-oxopropyl)phosphonate (17f)

To the solution of 17e (4.5 g, 6.55 mmol) in DCM (20 mL) was added TFA (10 mL), stirred at r.t. for 1 h. The reaction solution was concentrated and dissolved in 1,4-dioxane (30 mL) and water (10 mL). To this was added lithium hydroxide monohydrate (1.4 g, 32.65 mmol) in an ice bath, stirred at r.t. for 2 h. The reaction solution was purified by reverse phase column chromatography (Column: C18; water (0.5% ammonium bicarbonate)/acetonitrile, gradient elution, flow rate: 20 mL/min). The desired fraction was lyophilized to give the title compound 17f (3.3 g, 85.9% yield).

MS (ESI) m/z: 587.8 [M+H]+

### Step 5

### ((R)-2-amino-3-(((S)-1-(((S)-1-((4-(hydroxymethyl)phenyl)amino)-1-oxo-5-ureidopentan-2-yl)amino)-3-methyl-1-oxobutan-2-yl)amino)-3-oxopropyl)phosphonic acid (17 g)

To the solution of 17f (3.0 g, 5.11 mmol) in N-Methyl-2-pyrrolidinone (15 mL) was added sodium bromide (2.2 g, 21.48 mmol), trimethylsilyl trifluoromethanesulfonate (9.1 g, 40.91 mmol) was added dropwise in an ice bath, stirred at 60° C. overnight. The reaction solution was purified by reverse phase column chromatography (Column: C18; water (0.05% TFA)/acetonitrile, gradient elution, flow rate: 20 mL/min). The desired fraction was lyophilized to give the title compound 17g (1.5 g, 55.3% yield).

MS (ESI) m/z: 531.3 [M+H]+

### Step 6

### ((R)-1-(2,5-dioxo-2,5-dihydro-1H-pyrrol-1-yl)-14-(((S)-1-(((S)-1-((4-(hydroxymethyl)phenyl)amino)-1-oxo-5-ureidopentan-2-yl)amino)-3-methyl-1-oxobutan-2-yl)carbamoyl)-12-oxo-3,6,9-trioxa-13-azapentadecan-15-yl)phosphonic acid (17i)

To the solution of 17 h (500 mg, 0.94 mmol) and 17 g (413 mg, 1.04 mmol, commercial available) in DMF (10 mL) was added DIEA (365 mg, 2.83 mmol) in an ice bath, stirred at r.t. for 3 h. The reaction solution was purified by reverse phase column chromatography (Column: C18; water (0.05% TFA)/acetonitrile, gradient elution, flow rate: 20 mL/min). The desired fraction was lyophilized to give the title compound 17i (200 mg, 26.1% yield).

MS (ESI) m/z: 814.2 [M+H]+

### Step 7

### ((14R)-1-(2,5-dioxo-2,5-dihydro-1H-pyrrol-1-yl)-14-(((2S)-1-(((2S)-1-((4-((((3′-((ethoxycarbonyl)amino)-3-oxo-3H-spiro[isobenzofuran-1,9′-xanthen]-6′-yl)carbamoyl)oxy)methyl)phenyl)amino)-1-oxo-5-ureidopentan-2-yl)amino)-3-methyl-1-oxobutan-2-yl)carbamoyl)-12-oxo-3,6,9-trioxa-13-azapentadecan-15-yl)phosphonic acid (17)

To the solution of 13b (119 mg, 0.30 mmol) in DCM (6 mL) was added 2,6-lutidine (95 mg, 0.89 mmol), triphosgene (44 mg, 0.15 mmol) was added dropwise in an ice bath, stirred at r.t. for 4 h. The solution of 17i (200 mg, 0.25 mmol) in N-Methyl-2-pyrrolidinone was added dropwise to the reaction solution in an ice bath, stirred at r.t. overnight. The reaction solution was purified by flash column chromatography (DCM/MeOH, gradient elution). The crude product was purified by prep-HPLC (Column: Sunfire Prep C18 OBD 5 μm 19*250 mm; Mobile phase: water (0.05% TFA)/acetonitrile, gradient elution; flow rate: 20 mL/min). The desired fraction was lyophilized to give the title compound 17 (10 mg, 3.2% yield).

MS (ESI) m/z: 1242.5 [M+H]+

### Example 18

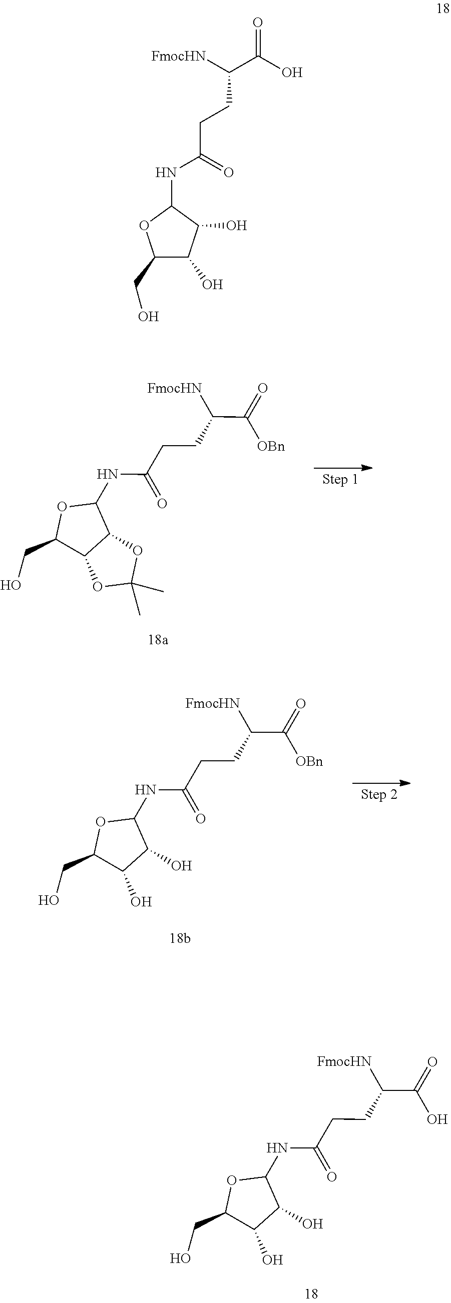

### Step 1

### Benzyl N2-(((9H-fluoren-9-yl)methoxy)carbonyl)-N5-((3R,4S,5R)-3,4-dihydroxy-5-(hydroxymethyl)tetrahydrofuran-2-yl)-L-glutaminate (18b)

To the solution of benzyl N2-(((9H-fluoren-9-yl)methoxy)carbonyl)-N5-((3aR,6R,6aR)-6-(hydroxymethyl)-2,2-dimethyltetrahydrofuro[3,4-d][1,3]dioxol-4-yl)-L-glutaminate 18a (100 mg, 0.16 mmol, available from WuXi AppTec) in THE (0.5 mL) were added water (1 mL) and TFA (1 mL) at 0° C., stirred at r.t. for 2 h. Toluene (3 mL) was added, concentrated under vacuum to give the title compound 18b (95 mg, crude, 100% yield), which was used directly without further purification.

MS (ESI) m/z: 591.5 [M+H]+

### Step 2

### N2-(((9H-fluoren-9-yl)methoxy)carbonyl)-N5-((3R,4S,5R)-3,4-dihydroxy-5-(hydroxymethyl)tetrahydrofuran-2-yl)-L-glutamine (18)

To the solution of 18b (94 mg, 0.16 mmol, crude) in methanol (2 mL) was added 10% Pd/C (17 mg) under nitrogen atmosphere. The mixture was stirred at r.t. for 1.5 h under hydrogen atmosphere (hydrogen balloon). The solution was filtered through Celite and concentrated to give the title compound 18 (80 mg, 100% yield)

MS (ESI) m/z: 499.2 [M−H]−

### Example 19

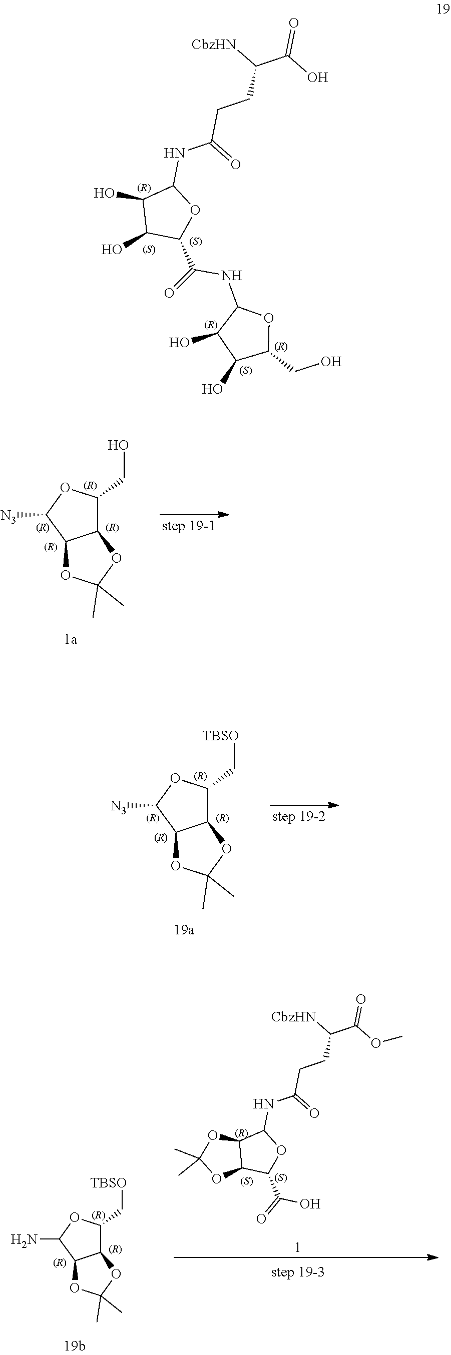

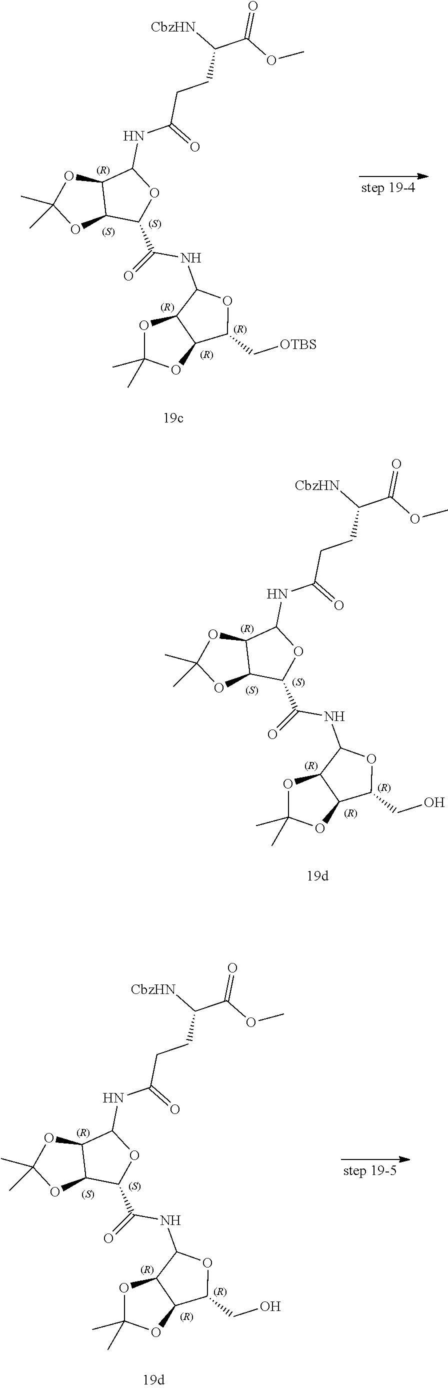

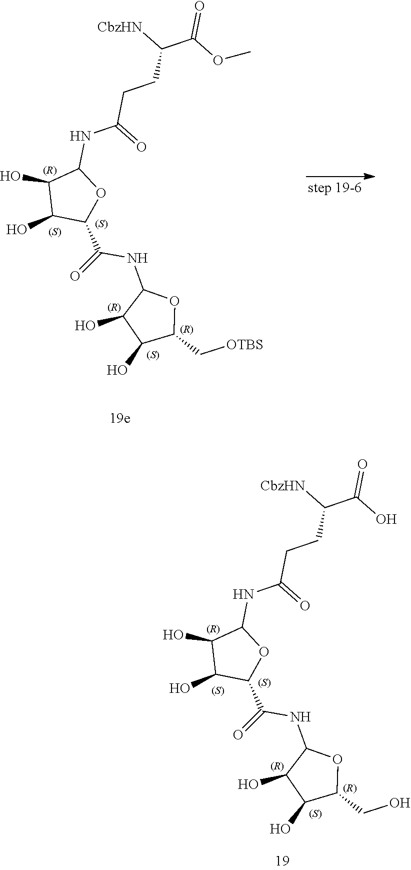

### Step 1

### (((3aR,4R,6R,6aR)-6-azido-2,2-dimethyltetrahydrofuro[3,4-d][1,3]dioxol-4-yl)methoxy)(tert-butyl)dimethylsilane (19a)

TBSCl (770.4 mg, 5.11 mmol) and Imidazole (632.7 mg, 9.29 mmol) were added to a mixture of 1a (1.00 g, 4.65 mmol) in DCM (10 mL). The mixture was reacted at r.t. for overnight and monitored by TLC. The reaction was quenched with 50 mL of sat·NaHCO3 and extracted with DCM (50 mL*2). After separation the combined organic layers were washed with brine (30 mL), dried over Na2SO4, filtered and the filtrate was concentrated under vacuum to give a residue. It was purified by silica gel column chromatography (A—Petroleum ether; B—EtOAc) to provide 19a (1.40 g, 91% yield)

### Step 2

### (3aR,4R,6R,6aR)-6-(((tert-butyldimethylsilyl)oxy)methyl)-2,2-dimethyltetrahydrofuro[3,4-d][1,3]dioxol-4-amine (19b)

Wet Pd/C (150 mg, 10% purity) was added to a mixture of 19a (1.40 g, 4.25 mmol) in MeOH (20 mL). The reaction mixture was purged with H2 balloon for three times and reacted at r.t. under H2 balloon for 1 h. After the reaction was completed, the mixture was filtered off through celite and washed with MeOH. The filtrate was concentrated under vacuum and purified by silica gel column chromatography (A—DCM; B—MeOH) to provide 19b (740.0 mg, 57.3% yield).

MS (ESI) m/z: 326.4 [M+Na+]

### Step 3

### (3aS,6R,6aR)-6-((S)-4-(((benzyloxy)carbonyl)amino)-5-methoxy-5-oxopentanamido)-2,2-dimethyltetrahydrofuro[3,4-d][1,3]dioxole-4-carboxylic acid (19c)

To a mixture of 1 (255.0 mg, 0.531 mmol) in Pyridine (1 mL) was added EDCI (101.7 mg, 0.531 mmol). The resulted yellow mixture was reacted at r.t. for 10 min. 19b (162.7 mg, 0.54 mmol) was added and reacted at the same temperature for another 4 h and monitored by LCMS. The mixture was concentrated under vacuum to remove most of pyridine then diluted with DMF (1 mL) and purified using prep-HPLC (Method: column: XBridge Prep C18 OBD Sum 19*250 mm; Mobile phase: A—water (0.1% formic acid): B—acetonitrile; Flow rate: 20 mL/min) to provide 19c (112.0 mg, 27% yield).

MS (ESI) m/z: 766.6 [M+H]+

### Step 4

### Methyl N2-((benzyloxy)carbonyl)-N5-((3aR,6S,6aS)-6-(((3aR,6R,6aR)-6-(hydroxymethyl)-2,2-dimethyltetrahydrofuro[3,4-d][1,3]dioxol-4-yl)carbamoyl)-2,2-dimethyltetrahydrofuro[3,4-d][1,3]dioxol-4-yl)-L-glutaminate (19d)

To a mixture of 19c (112.0 mg, 0.15 mmol) in THE (2 mL) was added TBAF (1M in THF, 161 μL, 0.161 mmol). The mixture was reacted at r.t. for 0.5 h. After the mixture was completed, the reaction was quenched with 10 mL of sat·NaHCO₃ and extracted with EtOAc (10 mL*2). After separation the combined organic layers were washed with brine (30 mL), dried over Na2SO4, filtered and the filtrate was concentrated under vacuum to provide 19d (95 mg, crude).

MS (ESI) m/z: 674.5 [M+Na+]

### Step 5

### Methyl N2-((benzyloxy)carbonyl)-N5-((2R,3R,5S)-5-(((2R,3R,5R)-3,4-dihydroxy-5-(hydroxymethyl)tetrahydrofuran-2-yl)carbamoyl)-3,4-dihydroxytetrahydrofuran-2-yl)-L-glutaminate (19e)

A mixture of 19d (112 mg, cured) in TFA/H2O (4:1, 1 mL) was reacted at r.t. for 0.5 h and monitored by LCMS. The mixture was concentrated under vacuum to provide 19e (83.0 mg, crude).

MS (ESI) m/z: 594.4[M+Na+]

### Step 6

### N2-((benzyloxy)carbonyl)-N5-((3R,4S,5S)-5-(((3R,4S,5R)-3,4-dihydroxy-5-(hydroxymethyl)tetrahydrofuran-2-yl)carbamoyl)-3,4-dihydroxytetrahydrofuran-2-yl)-L-glutamine (19)

To a mixture of 19e (83.0 mg, crude) in MeOH (1 mL) was added Lithium hydroxide aqueous solution (1N, 0.3 mL). The mixture was reacted at r.t. for 0.5 h. The mixture was acidified with pH=6, filtered and the filtrate was purified using prep-HPLC (Method: column: XBridge Prep C18 OBD Sum 19*250 mm; Mobile phase: A—water (0.1% formic acid): B—acetonitrile; Flow rate: 20 mL/min) to provide 19 (55 mg, 67.9% yield).

MS (ESI) m/z: 580.4[M+Na+]

### Example 20

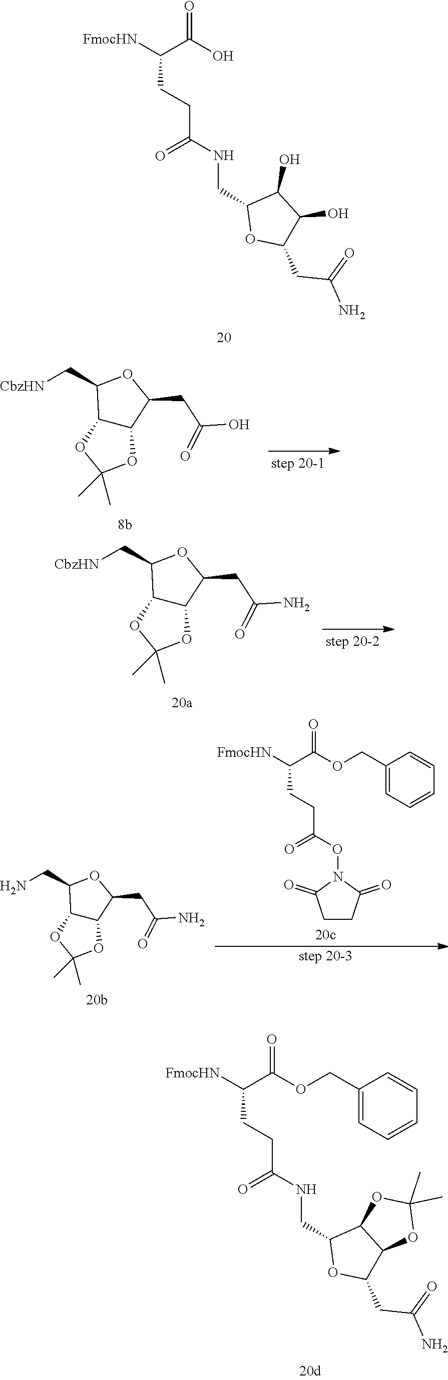

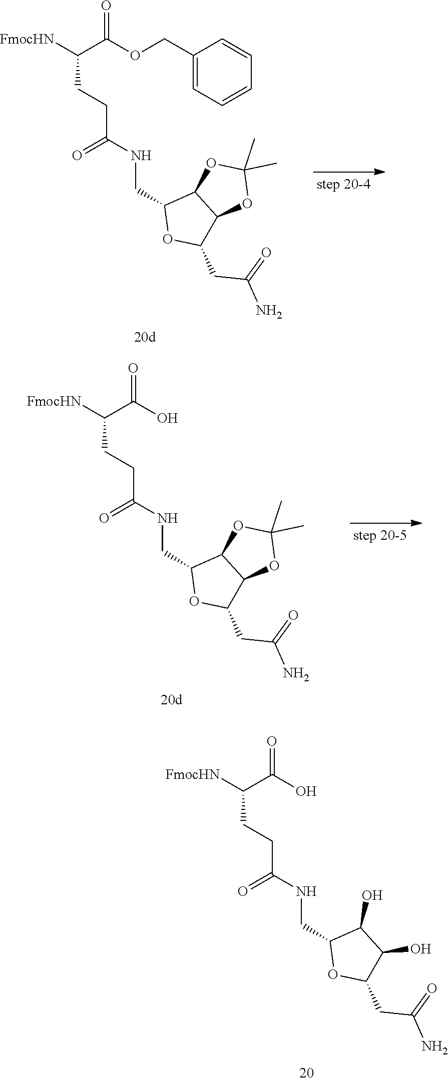

### Step 1

### Benzyl (((3aR,4R,6S,6aS)-6-(2-amino-2-oxoethyl)-2,2-dimethyltetrahydrofuro[3,4-d][1,3]dioxol-4-yl)methyl)carbamate (20a)

To a suspension of 8b (2.0 g, 5.47 mmol) in DMF (20 mL) were added PyBOP (3.42 g, 6.57 mmol), HOBt (887.60 mg, 6.57 mmol) and DIPEA (2.12 g, 16.42 mmol). The resulted brown suspension was reacted at 50° C. for overnight. The mixture was quenched with water (20 mL), extracted with EtOAc (30 mL*3). After separation the combined organics were washed with brine (30 mL*3), dried over Na2SO4, filtered and the filtrate was concentrated under vacuum to give a residue. It was purified by silica gel column chromatography (A—DCM; B—MeOH) to provide 20a (4.3 g, crude).

MS (ESI) m/z: 365.4 [M+H]+

### Step 2

### 2-((3aS,4S,6R,6aR)-6-(aminomethyl)-2,2-dimethyltetrahydrofuro[3,4-d][1,3]dioxol-4-yl)acetamide (20b)

Compound 20b (1.9 g, crude) was synthesized according to synthetic procedure of step 2 of example 19.

MS (ESI) m/z: 231.4 [M+H]+

### Step 3

### Benzyl N2-(((9H-fluoren-9-yl)methoxy)carbonyl)-N5-(((3aR,4R,6S,6aS)-6-(2-amino-2-oxoethyl)-2,2-dimethyltetrahydrofuro[3,4-d][1,3]dioxol-4-yl)methyl)-L-glutaminate (20d)

To a mixture of 20b (900.0 mg, crude) and 20c [(1.74 g, 3.13 mmol); commercial] in THE (20 mL) was added sat NaHCO₃ (5 mL). The mixture was reacted at r.t. for 1 h. After the reaction was completed, the mixture was diluted with water (20 mL), extracted with EtOAc (20 mL*2). The combined organics were washed with brine (30 mL), dried over Na2SO4, filtered and the filtrate was concentrated under vacuum to provide 20d (2.1 g, crude).

MS (ESI) m/z: 672.6 [M+H]+

### Step 4

### N2-(((9H-fluoren-9-yl)methoxy)carbonyl)-N5-(((3aR,4R,6S,6aS)-6-(2-amino-2-oxoethyl)-2,2-dimethyltetrahydrofuro[3,4-d][1,3]dioxol-4-yl)methyl)-L-glutamine (20e)

Compound 20e (1.87 g, crude) was synthesized according to synthetic procedure of step 2 of example 19.

MS (ESI) m/z: 582.5 [M+H]+

### Step 5

### N2-(((9H-fluoren-9-yl)methoxy)carbonyl)-N5-(((2R,3S,4R,5S)-5-(2-amino-2-oxoethyl)-3,4-dihydroxytetrahydrofuran-2-yl)methyl)-L-glutamine (20)

Compound 20 (630.0 g, 42.5% yield) was synthesized according to synthetic procedure of step 5 of example 19.

MS (ESI) m/z: 542.5 [M+H]+

### Example 21

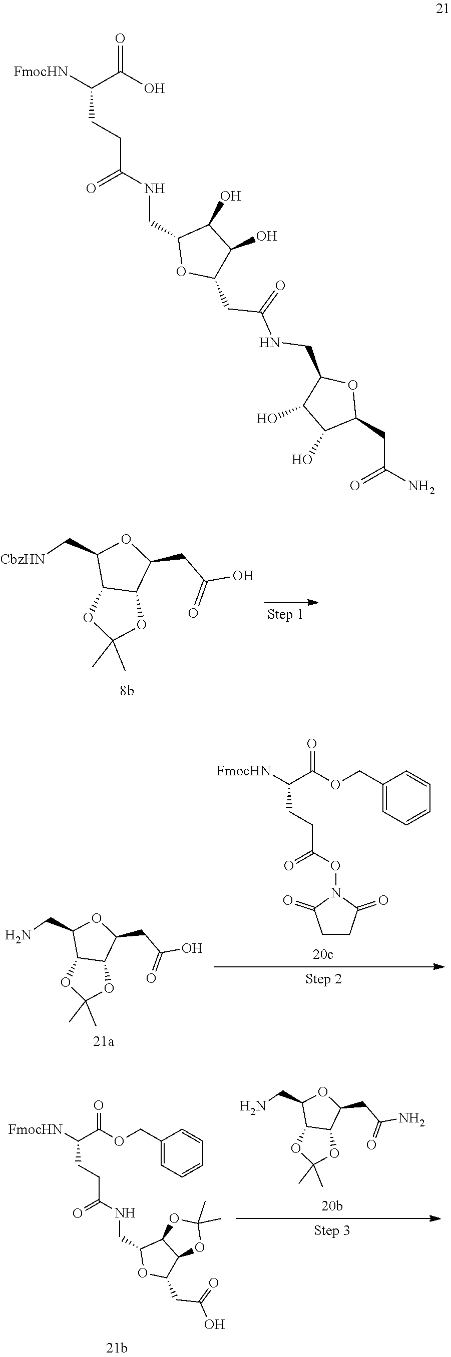

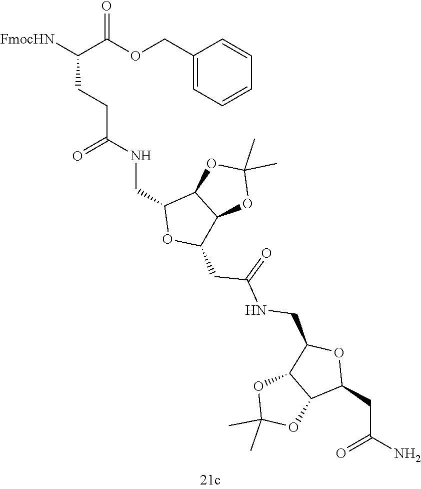

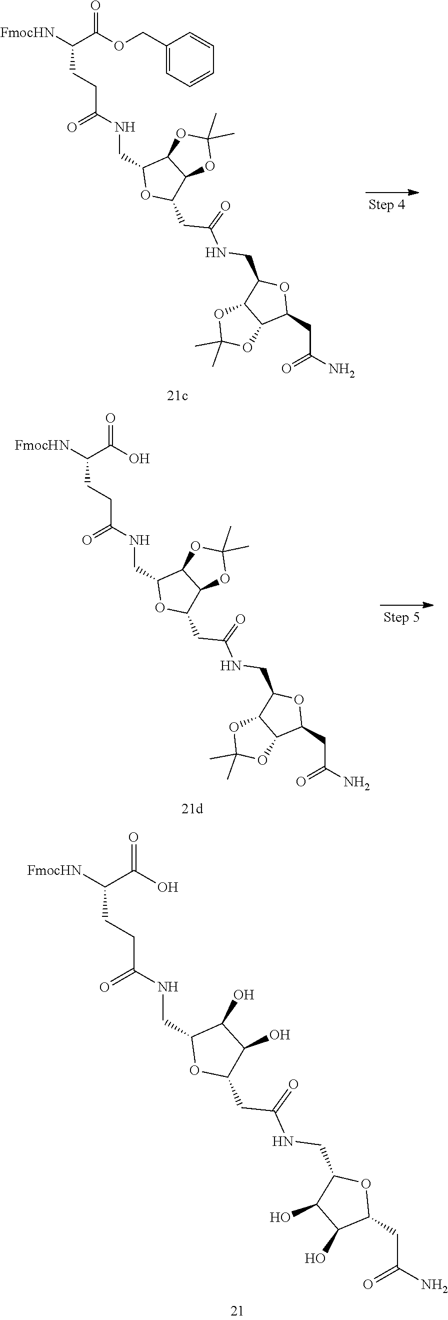

### Step 1

### 2-((3aS,4S,6R,6aR)-6-(aminomethyl)-2,2-dimethyltetrahydrofuro[3,4-d][1,3]dioxol-4-yl)acetic acid (21a)

Compound 21a (480 mg, 75% yield) was synthesized according to synthetic procedure of step 2 of example 19.

MS (ESI) m/z: 232.4 [M+H]+

### Step 2

### 2-((3aS,4S,6R,6aR)-6-(((S)-4-((((9H-fluoren-9-yl)methoxy)carbonyl)amino)-5-(benzyloxy)-5-oxopentanamido)methyl)-2,2-dimethyltetrahydrofuro[3,4-d][1,3]dioxol-4-yl)acetic acid (21b)

Compound 21b (1.5 g, crude) was synthesized according to synthetic procedure of step 3 of example 20.

MS (ESI) m/z 673.4 [M+H]+

### Step 3

### Benzyl N2-(((9H-fluoren-9-yl)methoxy)carbonyl)-N5-(((3aR,4R,6S,6aS)-6-(2-((((3aR,4R,6S,6aS)-6-(2-amino-2-oxoethyl)-2,2-dimethyltetrahydrofuro[3,4-d][1,3]dioxol-4-yl)methyl)amino)-2-oxoethyl)-2,2-dimethyltetrahydrofuro[3,4-d][1,3]dioxol-4-yl)methyl)-L-glutaminate (21c)

To a mixture of 21b (410 mg, 0.609 mmol) in DMF (6 mL) were added HATU (231.73 mg, 0.609 mmol) and DIPEA (236.3 mg, 1.828 mmol). The resulted yellow mixture was reacted at r.t. for 10 min then 20b (168.4 mg, 0.731 mmol) was added. The mixture was reacted at r.t. for another 1 h. After the reaction was completed, the mixture was quenched with 20 mL waters, extracted with EtOAc (30 mL*2). After separation the combined organic layers were washed with brine (30 mL*3), dried over Na2SO4, filtered and the filtrate was concentrated under vacuum to give a residue. It was purified by C18 column flash chromatography [Mobile phase: A—water (0.1% formic acid): B—acetonitrile] to provide 21c (430.0 mg, 66.7% yield).

MS (ESI) m/z: 885.4 [M+H]+

### Step 4

### N2-(((9H-fluoren-9-yl)methoxy)carbonyl)-N5-(((3aR,4R,6S,6aS)-6-(2-((((3aR,4R,6S,6aS)-6-(2-amino-2-oxoethyl)-2,2-dimethyltetrahydrofuro[3,4-d][1,3]dioxol-4-yl)methyl)amino)-2-oxoethyl)-2,2-dimethyltetrahydrofuro[3,4-d][1,3]dioxol-4-yl)methyl)-L-glutamine (21d)

Compound 21d (377.5 g, crude) was synthesized according to synthetic procedure of step 2 of example 19.

MS (ESI) m/z: 795.4 [M+H]+

### Step 5

### N2-(((9H-fluoren-9-yl)methoxy)carbonyl)-N5-(((2R,3S,4R,5S)-5-(2-((((2R,3S,4R,5S)-5-(2-amino-2-oxoethyl)-3,4-dihydroxytetrahydrofuran-2-yl)methyl)amino)-2-oxoethyl)-3,4-dihydroxytetrahydrofuran-2-yl)methyl)-L-glutamine (21)

Compound 21 (181.0 mg, 53.1% yield) was synthesized according to synthetic procedure of step 5 of example 19.

MS (ESI) m/z: 715.5 [M+H]+

### Example 22

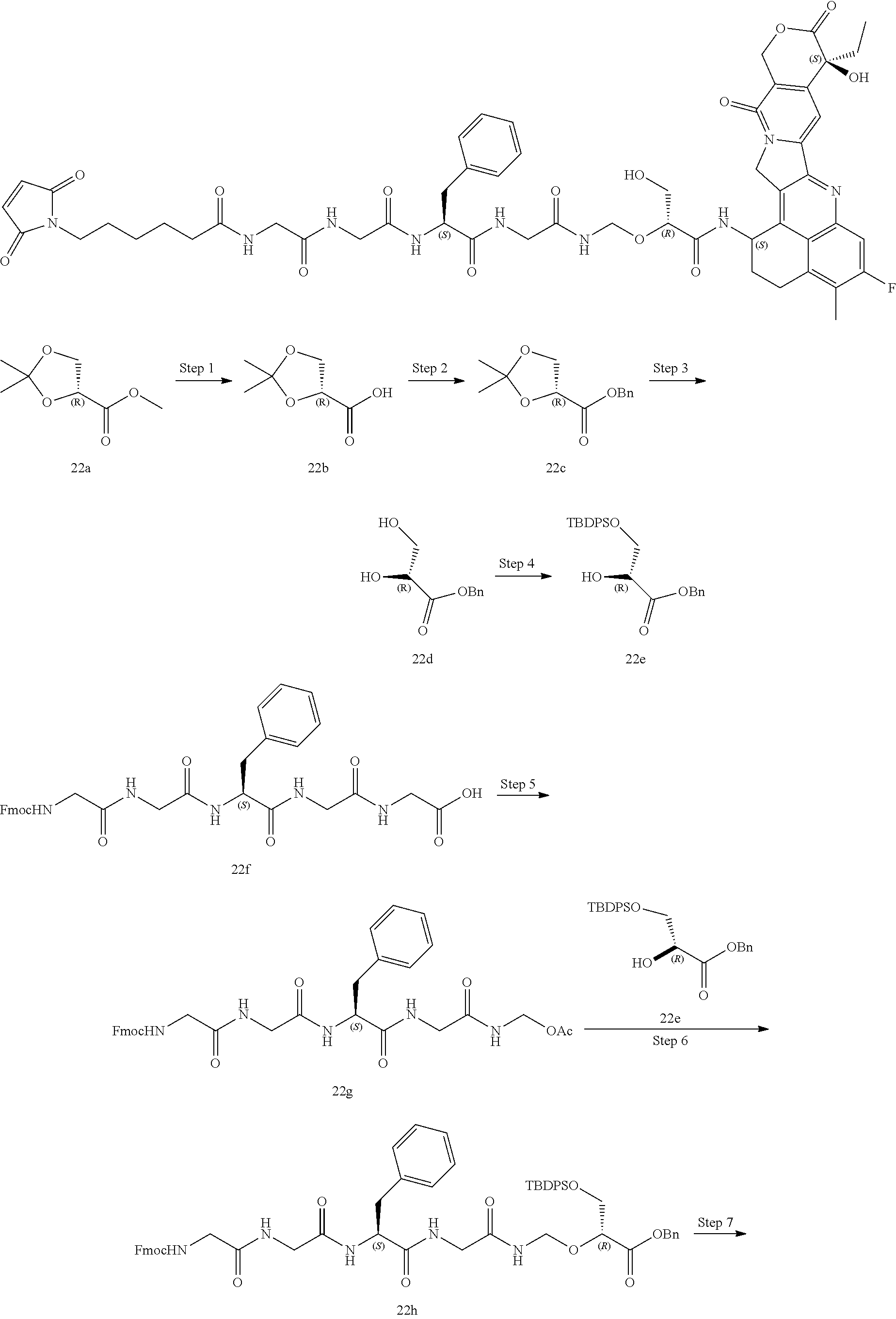

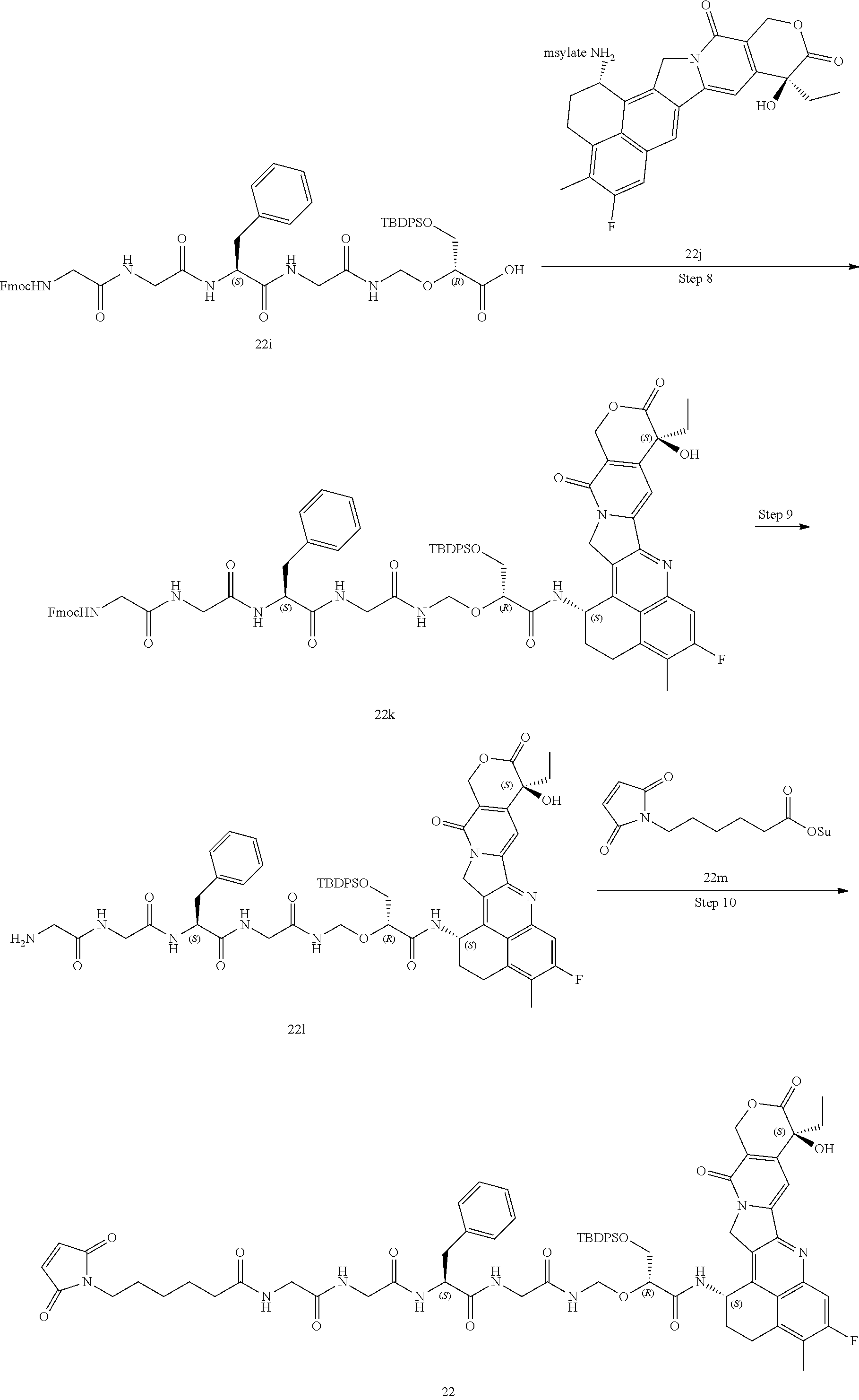

### Step 1

### (R)-2,2-dimethyl-1,3-dioxolane-4-carboxylic acid (22b)

22a (2 g, 12.49 mmol) was dissolved in a mixed solvent of THF and H2O (v/v=1:1, 40 mL) followed by the addition of LiOH (449 mg, 18.73 mmol). The resulting mixture was stirred at 25° C. for 1 h. After complete reaction, the reaction mixture was extracted with EA (30 mL). The remaining water layer was acidified to pH=3 with aqueous HCl (1 M) and further extracted with EA (30 mL*3). The combined organic layers were dried over anhydrous Na2SO4, filtered and concentrated under reduced pressure to afford 22b (1.51 g, 82.8% yield) as a clear oil which was used directly in next step without purification.

1H NMR: δ 11.15 (br s, 1H), 4.63 (dd, J=7.6, 4.8 Hz, 1H), 4.30 (dd, J=8.8, 7.6 Hz, 1H), 4.19 (dd, J=8.8, 4.8 Hz, 1H), 1.53 (s, 3H), 1.42 (s, 3H).

### Step 2

### Benzyl (R)-2,2-dimethyl-1,3-dioxolane-4-carboxylate (22c)

22b (3 g, 20.53 mmol) was dissolved in DMF (21 mL) followed by the addition of BnBr (5.27 g, 30.79 mmol) and K2CO3 (4.26 g, 30.79 mmol). The resulting mixture was stirred at 25° C. for 2 h. After complete reaction, the reaction mixture was diluted with H2O (30 mL) and extracted with EA (40 mL*3). The combined organic layers were dried over anhydrous Na2SO4, filtered and concentrated under reduced pressure to afford a brown oil as the crude product which was purified by flash column (PE/EA=100/0 to 80/20) to afford 22c as a clear oil (2.52 g, 52.0% yield).

1H NMR: δ 7.38-7.31 (m, 5H), 5.21 (m, 2H), 4.62 (dd, J=7.2, 5.2 Hz, 1H), 4.30 (dd, J=8.8, 7.2 Hz, 1H), 4.19 (dd, J=8.8, 5.2 Hz, 1H), 1.49 (s, 3H), 1.40 (s, 3H).

### Step 3

### Benzyl (R)-2,3-dihydroxypropanoate (22d)

22c (1 g, 4.23 mmol) was dissolved in DCM (13 mL) followed by the addition of a mixed solvent of TFA and H2O (v/v=9:1, 13 mL) at 0° C. The resulting mixture was stirred at 0° C. for 1 h. After complete reaction, the reaction mixture was concentrated under reduced pressure and purified by flash column (PE/EA=95/5 to 20/80) to afford 22d (557 mg, 67.1% yield) as a colorless oil.

1H NMR: δ 7.40-7.31 (m, 5H), 5.25 (d, J=2.8 Hz, 2H), 4.31 (t, J=3.6 Hz, 1H), 3.93-3.85 (m, 2H), 2.89 (br s, 2H).

### Step 4

### Benzyl (R)-3-((tert-butyldiphenylsilyl)oxy)-2-hydroxypropanoate (22e)

22d (557 mg, 2.84 mmol) was dissolved in anhydrous DMF (8 mL) followed by the addition of imidazole (290 mg, 4.26 mmol) and TBDPSCl (858 mg, 3.12 mmol). The resulting mixture was stirred at 25° C. for 4 h. After complete reaction, the reaction mixture was diluted with EA (50 mL) and washed with brine (25 mL*2) and water (25 mL*3). The organic layer was dried over anhydrous Na2SO4, filtered and concentrated under reduced pressure to afford a yellow oil as the crude product which was purified by flash column (PE/EA=100/0 to 75/25) to afford 22e as a clear oil (705 mg, 57.1% yield).

MS (ESI) m/z: 457.6 [M+Na]+

### Step 5

### (S)-11-benzyl-1-(9H-fluoren-9-yl)-3,6,9,12,15-pentaoxo-2-oxa-4,7,10,13,16 pentaazaheptadecan-17-yl acetate (22 g)

22f (commercially available, 5 g, 8.12 mmol) was dissolved in anhydrous DMF (26 mL) followed by successive addition of Cu(OAc)2 (561 mg, 3.09 mmol), Pb(OAc)4 (4.11 g, 9.26 mmol) and HOAc (1.11 g, 18.44 mmol). The resulting mixture was stirred at 60° C. under N2 atmosphere for 70 min. After complete reaction, the reaction mixture was diluted with DCM (160 mL) and washed with brine (40 mL*3) and H2O (40 mL*2). The organic layer was dried over anhydrous Na2SO4, filtered and concentrated under reduced pressure to afford a brown oil as the crude product which was purified by flash column (DCM/MeOH=100/0 to 90/10) to afford 22 g as a white solid (4.19 g, 81.8% yield).

MS (ESI) m/z: 652.5 [M+Na]+

### Step 6

### Benzyl (11S,19R)-11-benzyl-19-(((tert-butyldiphenylsilyl)oxy)methyl)-1-(9H-fluoren-9-yl)-3,6,9,12,15-pentaoxo-2,18-dioxa-4,7,10,13,16-pentaazaicosan-20-oate (22 h)

22 g (150 mg, 0.24 mmol), 22e (207 mg, 0.48 mmol) and 4 Å molecular sieve (200 mg) were added into anhydrous THE (3 mL). The resulting mixture was stirred at 25° C. for 30 min before addition of Sc(OTf)3 (117 mg, 0.24 mmol). The mixture was further stirred at 25° C. for 16 h. After complete reaction, the reaction mixture was filtered and concentrated under reduced pressure to afford a yellow oil as the crude product which was purified by flash column (DCM/MeOH=100/0 to 90/10) to afford 22 h as a white solid (174 mg, 72.6% yield).

MS (ESI) m/z: 1026.5 [M+Na]+

### Step 7

### (11S,19R)-11-benzyl-19-(((tert-butyldiphenylsilyl)oxy)methyl)-1-(9H-fluoren-9-yl)-3,6,9,12,15-pentaoxo-2,18-dioxa-4,7,10,13,16-pentaazaicosan-20-oic acid (22i)

22 h (150 mg, 0.15 mmol) was dissolved in a mixture solvent of THF (2.5 mL) and H2O (2.5 mL) followed by the addition of Pd/C (wet, 10%, 45 mg). The mixture was stirred at 25° C. under H2 (15 psi) for 4 h. After complete reaction, the mixture was filtered and concentrated under reduced pressure to afford 22i (136 mg, quant.) as a white solid which was used directly in next step without purification.

MS (ESI) m/z: 936.6 [M+Na]+

### Step 8

### (9H-fluoren-9-yl)methyl ((6R,14S)-14-benzyl-6-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)-2,2-dimethyl-10,13,16,19-tetraoxo-3,3-diphenyl-4,7-dioxa-9,12,15,18-tetraaza-3-silaicosan-20-yl)carbamate (22k)

22i (85 mg, 0.09 mmol), HATU (42 mg, 0.11 mmol) and DIEA (36 mg, 0.28 mmol) were dissolved in DMF (2 mL). The mixture was stirred at 25° C. for 15 min before the addition of Exatecan mesylate (22j, commercially available, 59 mg, 0.11 mmol). The mixture was further stirred at 25° C. for 30 min. After complete reaction, the mixture was filtered and purified by prep-HPLC (Method: column: XBridge Prep C18 OBD Sum 19*250 mm; Mobile phase: A—water (0.1% formic acid): B—acetonitrile; Flow rate: 20 mL/min) to afford 22k (80 mg, 64.6% yield) as a pale yellow solid.

MS (ESI) m/z: 1355.0 [M+Na]+

### Step 9

### (R)-2-(((S)-13-amino-7-benzyl-3,6,9,12-tetraoxo-2,5,8,11-tetraazatridecyl)oxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxypropanamide (221)

22k (78 mg, 0.06 mmol) was dissolved in anhydrous THE (6 mL) followed by the addition of TBAF (1 M solution in THF, 70 μL, 0.07 mmol). The resulting mixture was stirred at 25° C. for 1 h and Et2NH (60 μL, 0.58 mmol) was added. The mixture was further stirred at 25° C. for 1 h. After complete reaction, the reaction mixture was purified by prep-HPLC (Method: column: XBridge Prep C18 OBD Sum 19*250 mm; Mobile phase: A—water (0.1% formic acid): B—acetonitrile; Flow rate: 20 mL/min) to afford 221 (26 mg, 51.0% yield) as a white solid.

MS (ESI) m/z: 871.6 [M+H]+

### Step 10

### N-((2R,10S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-(hydroxymethyl)-1,6,9,12,15-pentaoxo-3-oxa-5,8,11,14-tetraazahexadecan-16-yl)-6-(2,5-dioxo-2,5-dihydro-1H-pyrrol-1-yl)hexanamide (22)

22l (24 mg, 0.028 mmol) and 22m (9.4 mg, 0.030 mmol) were dissolved in DMF (0.5 mL) followed by the addition of TEA (2.5 mg, 0.025 mmol). The resulting mixture was stirred at 25° C. for 30 min. After complete reaction, the reaction mixture was purified by prep-HPLC (Method: column: XBridge Prep C18 OBD Sum 19*250 mm; Mobile phase: A—water (0.1% formic acid): B—acetonitrile; Flow rate: 20 mL/min) to afford 22 (16.5 mg, 51.2% yield) as a white solid.

MS (ESI) m/z: 1064.7 [M+H]+

### Example 23

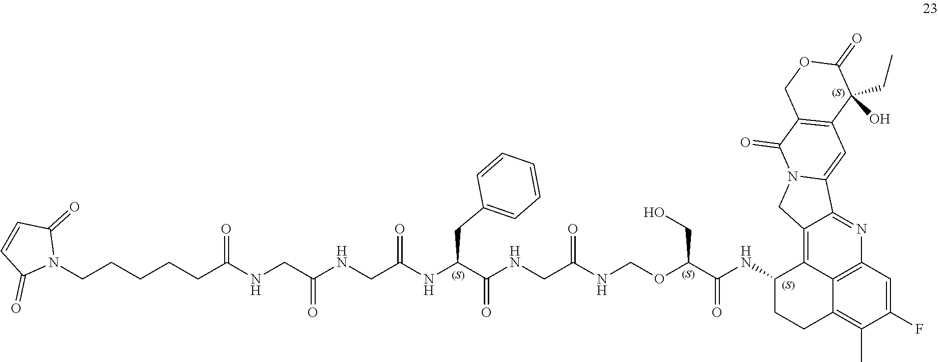

Compound 23 was synthesized according to the synthetic procedure of example 22.

MS (ESI) m/z: 1064.7 [M+H]+

### Example 24

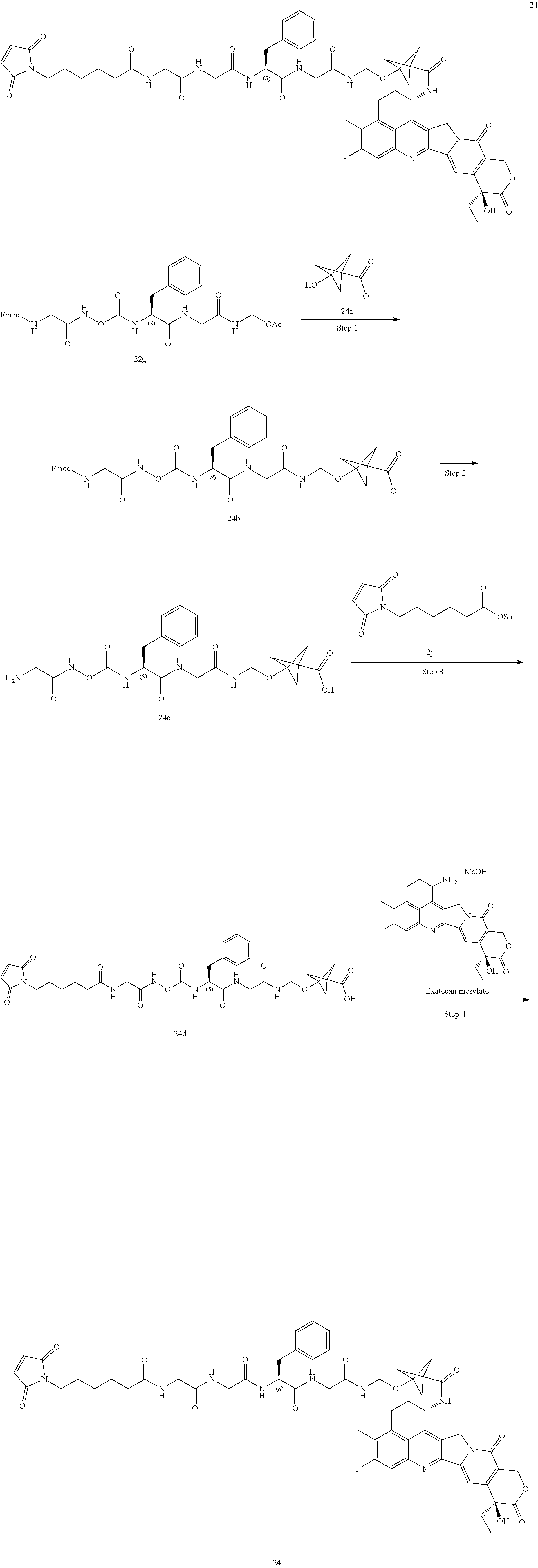

### Step 1

### Methyl (S)-3-((11-benzyl-1-(9H-fluoren-9-yl)-3,6,9,12,15-pentaoxo-2-oxa-4,7,10,13,16-pentaazaheptadecan-17-yl)oxy)bicyclo[1.1.1]pentane-1-carboxylate (24b)

To the mixture of 22 g (50 mg, 0.08 mmol), methyl 3-hydroxybicyclo[1.1.1]pentane-1-carboxylate 24a (23 mg, 0.16 mmol) and dried 4 Å molecular sieves (140 mg) in dry THF (1 mL) was added scandium trifluoromethanesulfonate (47 mg, 0.1 mmol), stirred at r.t. overnight. The solution was filtered through celite, concentrated and purified by flash column chromatography (DCM/MeOH=10/1) to give the title compound 24b (41 mg, 72.5% yield) as a white solid.

MS (ESI) m/z: 734.5 [M+Na]+

### Step 2

### (S)-3-((13-amino-7-benzyl-3,6,9,12-tetraoxo-2,5,8,11-tetraazatridecyl)oxy)bicyclo[1.1.1]pentane-1-carboxylic acid (24c)

To the solution of 24b (41 mg, 0.06 mmol) in methanol (1 mL) and water (0.2 mL) was added potassium carbonate (83 mg, 0.6 mmol), stirred at r.t. for 2 h. The reaction solution was extracted with hexane (2 mL*3) and the pH of the water phase was adjusted to 6 with 2N HCl. The aqueous solution was concentrated and lyophilized to give the title compound 24c (230 mg, crude), which was used directly without further purification.

MS (ESI) m/z: 474.3 [M−H]−

### Step 3

### (S)-3-((7-benzyl-20-(2,5-dioxo-2,5-dihydro-1H-pyrrol-1-yl)-3,6,9,12,15-pentaoxo-2,5,8,11,14-pentaazaicosyl)oxy)bicyclo[1.1.1]pentane-1-carboxylic acid (24d)

To the solution of 24c (230 mg, crude, theoretical amount 27 mg, 0.06 mmol) and 2j (35 mg, 0.11 mmol) in dry DMF (1 mL) was added DIEA (47 μL, 0.28 mmol), stirred at r.t. for 15 min. AcOH (40 L) was added to quench the reaction. The solution was purified by prep-HPLC (0.1% FA in water/MeCN), lyophilized to give the title compound 24d (24 mg, 63.0% yield) as a white solid.

MS (ESI) m/z: 667.4 [M−H]−

### Step 4

### 3-(((S)-7-benzyl-20-(2,5-dioxo-2,5-dihydro-1H-pyrrol-1-yl)-3,6,9,12,15-pentaoxo-2,5,8,11,14-pentaazaicosyl)oxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)bicyclo[1.1.1]pentane-1-carboxamide (24)

To the solution of 24d (24 mg, 35.9 mol) and NHS (6.2 mg, 53.8 mol) in dry DMF (0.3 mL) was added DCC (11 mg, 53.8 mol), stirred at r.t. overnight. The reaction solution was added slowly into the solution of exatecan mesylate (23 mg, 43.1 mol) and DIEA (8 μL, 43.1 mol) in dry DMF (0.3 mL), stirred at r.t. overnight. The reaction solution was filtered and purified by prep-HPLC (0.1% FA in water/MeCN), lyophilized to give the title compound 24 (2.6 mg, 10.7% yield) as a white solid.

MS (ESI) m/z: 1086.9 [M+H]+

### Example 25

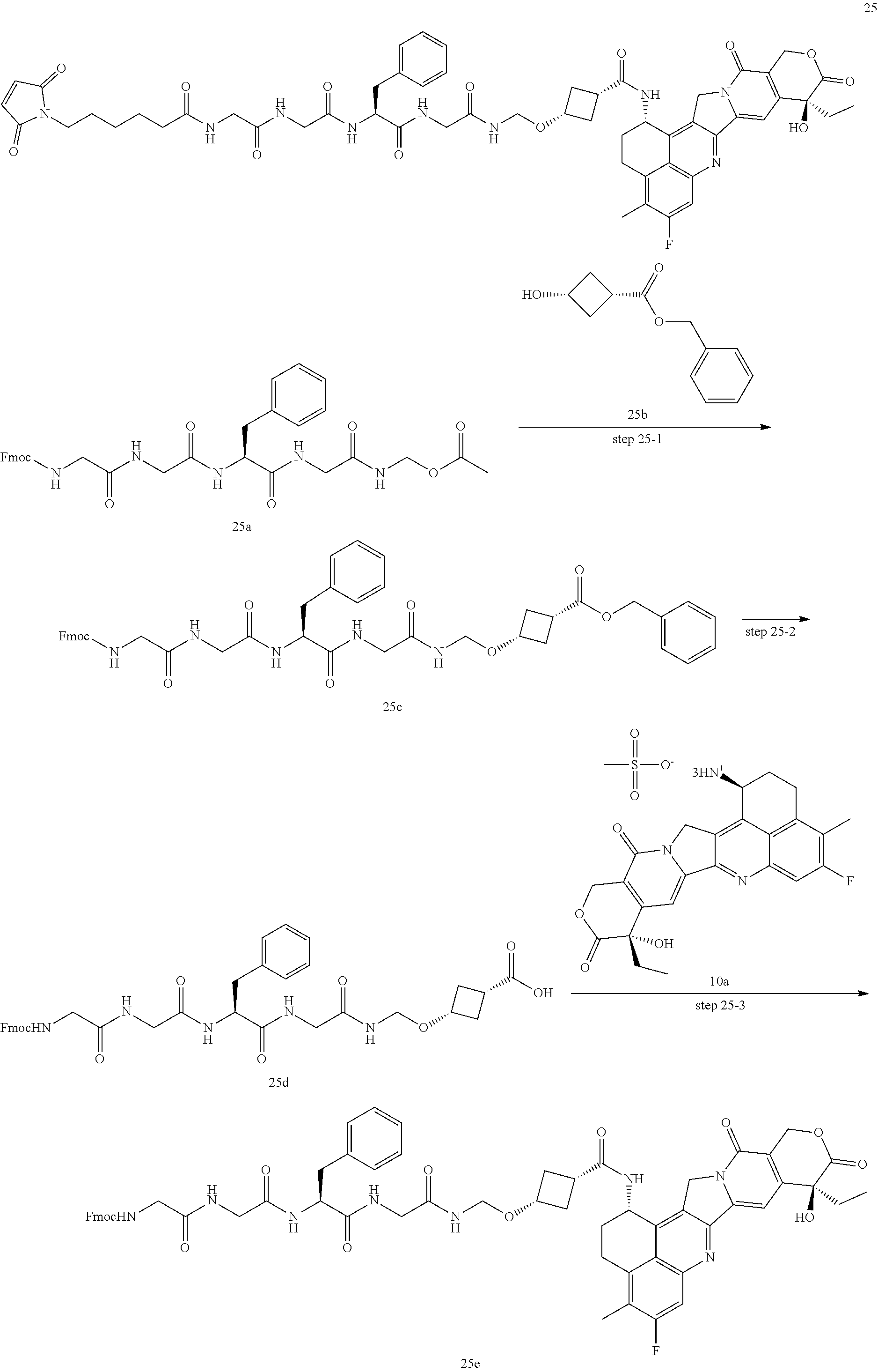

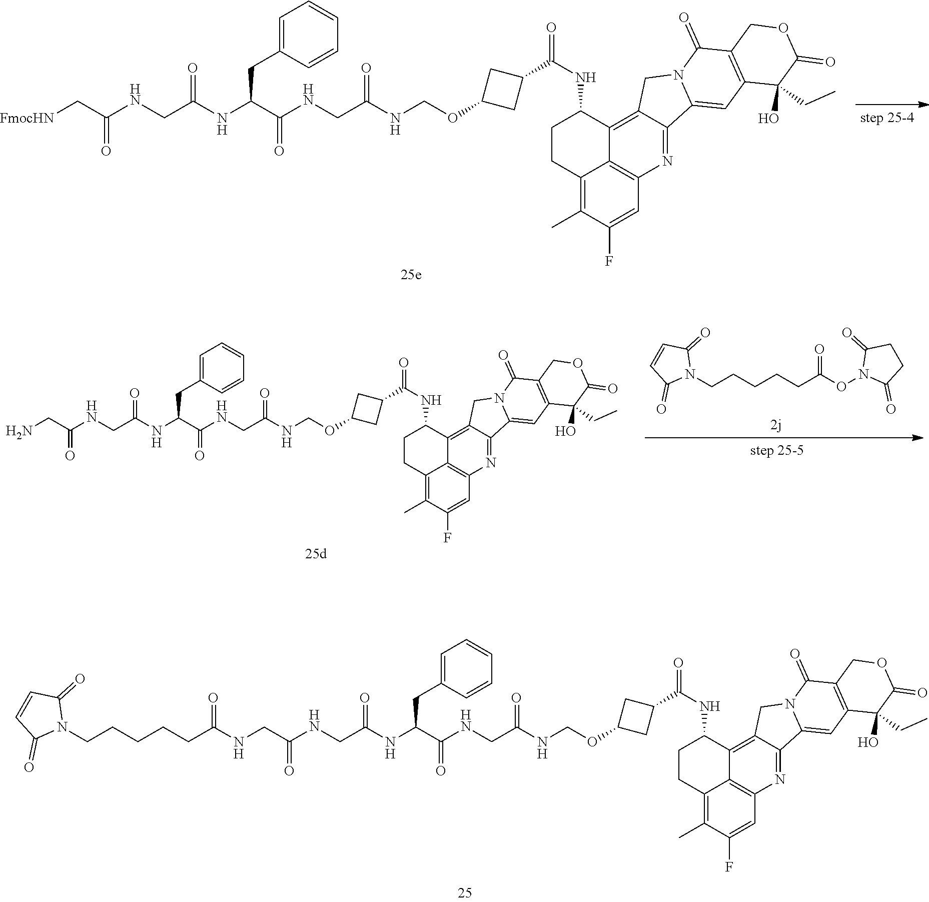

### Step 1

### Benzyl (1R,3s)-3-(((S)-11-benzyl-1-(9H-fluoren-9-yl)-3,6,9,12,15-pentaoxo-2-oxa-4,7,10,13,16-pentaazaheptadecan-17-yl)oxy)cyclobutane-1-carboxylate (25c)

To solution of 25a (100 mg, 0.159 mmol) and 25b (31.76 mg, 0.318 mmol) in THE (1.5 mL) was added 4 Å molecular sieve. The mixture was stirred at r.t. for 10 min then Sc(OTf)3 (78.16 mg, 0.159 mmol) was added and reacted at r.t. for another 16 h. The suspension mixture was filtered through a pad of celite and the cake was washed with THE (30 mL) then the filtrate was quenched by addition of Sat·NaHCO3 (30 mL), extracted with EtOAc (30 mL*2). After separation the combined organic layers were washed with brine (50 mL), dried over Na2SO4, filtered and the filtrate was concentrated under vacuum to give a residue. It was purified by silica gel column chromatography (A—DCM; B—MeOH) to provide 25c (115 mg, 93.3% yield).

MS (ESI) m/z: 798.5 [M+Na+]

### Step 2

### (1R,3S)-3-(((S)-11-benzyl-1-(9H-fluoren-9-yl)-3,6,9,12,15-pentaoxo-2-oxa-4,7,10,13,16-pentaazaheptadecan-17-yl)oxy)cyclobutane-1-carboxylic acid (25d)

To a solution of 25c (115 mg, 0.183 mmol) in MeOH (2 mL) was added wet Pd/C (25 mg). The black suspension was purged with H2 balloon for three times then reacted at r.t. for 2 h under H2 balloon. After the reaction was completed the black suspension was filtered off through a pad of celite and the cake wash with MeOH, the combined organic layers were concentrated under vacuum to provide 25d (90 mg, 63.5% yield).

MS (ESI) m/z: 708.5 [M+Na+]

### Step 3

### (9H-fluoren-9-yl)methyl ((S)-7-benzyl-1-((1R,3R)-3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)cyclobutoxy)-3,6,9,12-tetraoxo-2,5,8,11-tetraazatridecan-13-yl)carbamate (25e)

To a mixture of 25d (80 mg, 0.117 mmol) and TSTU (35.15 mg, 0.117 mmol) in DMF (2 mL) was added DIPEA (30.18 mg, 0.233 mmol). The mixture was reacted at r.t. for 10 min and the acid was converted to active ester based on LCMS. 10a (68.1 mg, 0.128 mmol) was added and reacted at the same temperature for another 5 hr. The reaction The mixture was filtered and the filtrate was purified using prep-HPLC (Method: column: XBridge Prep C18 OBD Sum 19*250 mm; Mobile phase: A—water (0.1% formic acid): B—acetonitrile; Flow rate: 20 mL/min) to provide 25e (55 mg, 42.6% yield).

MS (ESI) m/z: 1125.7 [M+Na+]

### Step 4

### (1R,3R)-3-(((S)-13-amino-7-benzyl-3,6,9,12-tetraoxo-2,5,8,11-tetraazatridecyl)oxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)cyclobutane-1-carboxamide (25f)

To a solution of 25e (55 mg, 0.050 mmol) in DMF (1 mL) was added piperidine (85.15 mg, 1.0 mmol). The mixture was reacted at r.t. for 10 min. The reaction The mixture was filtered and the filtrate was purified using prep-HPLC (Method: column: XBridge Prep C18 OBD Sum 19*250 mm; Mobile phase: A—water (0.1% formic acid): B—acetonitrile; Flow rate: 20 mL/min) to provide 25f (35 mg, 80% yield).

MS (ESI) m/z: 903.7 [M+Na+]

### Step 5

### (1R,3R)-3-(((S)-7-benzyl-20-(2,5-dioxo-2,5-dihydro-1H-pyrrol-1-yl)-3,6,9,12,15-pentaoxo-2,5,8,11,14-pentaazaicosyl)oxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)cyclobutane-1-carboxamide (25)

Compound 25 (12.5 mg, 68.2% yield) was synthesized according to synthetic procedure of step 8 of example 2.

### Example 26

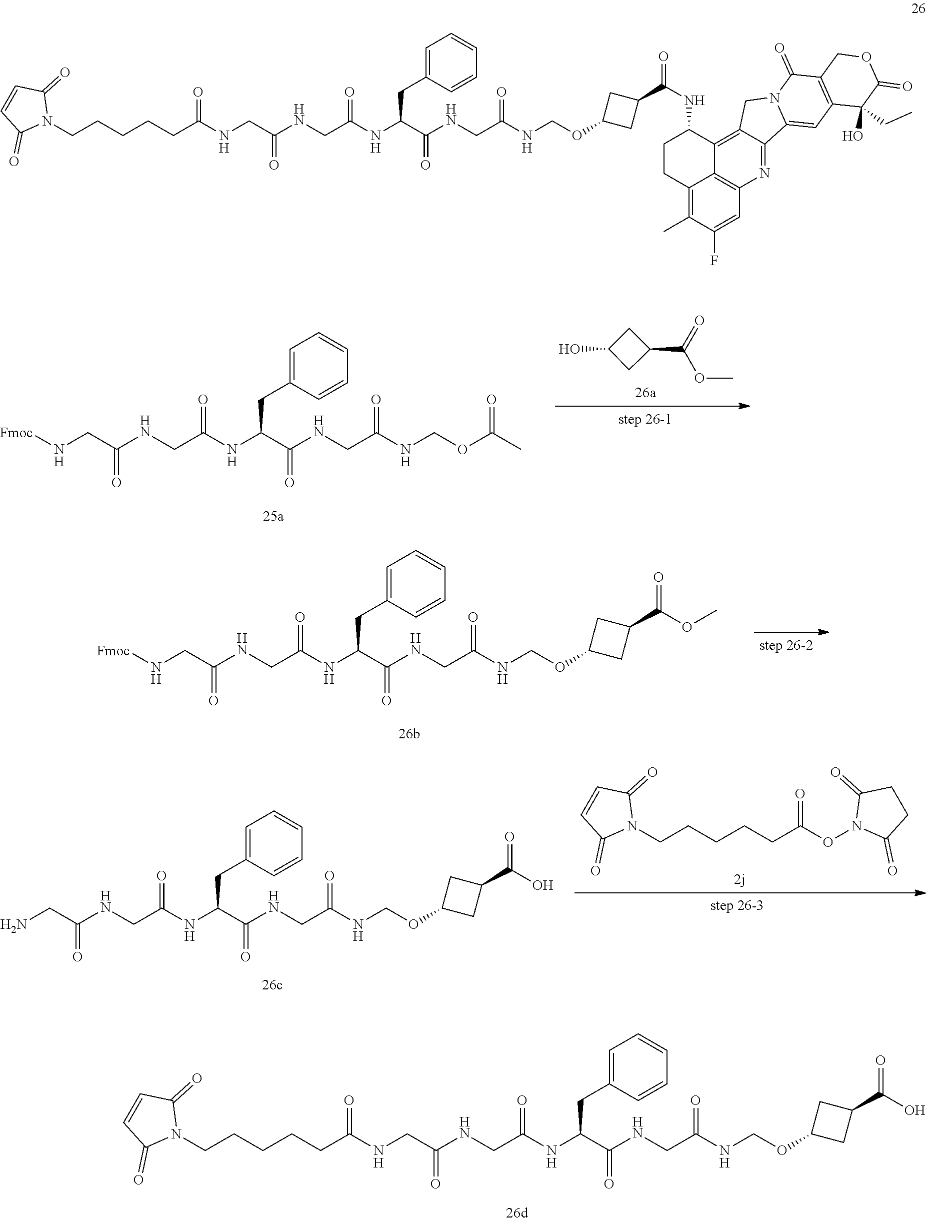

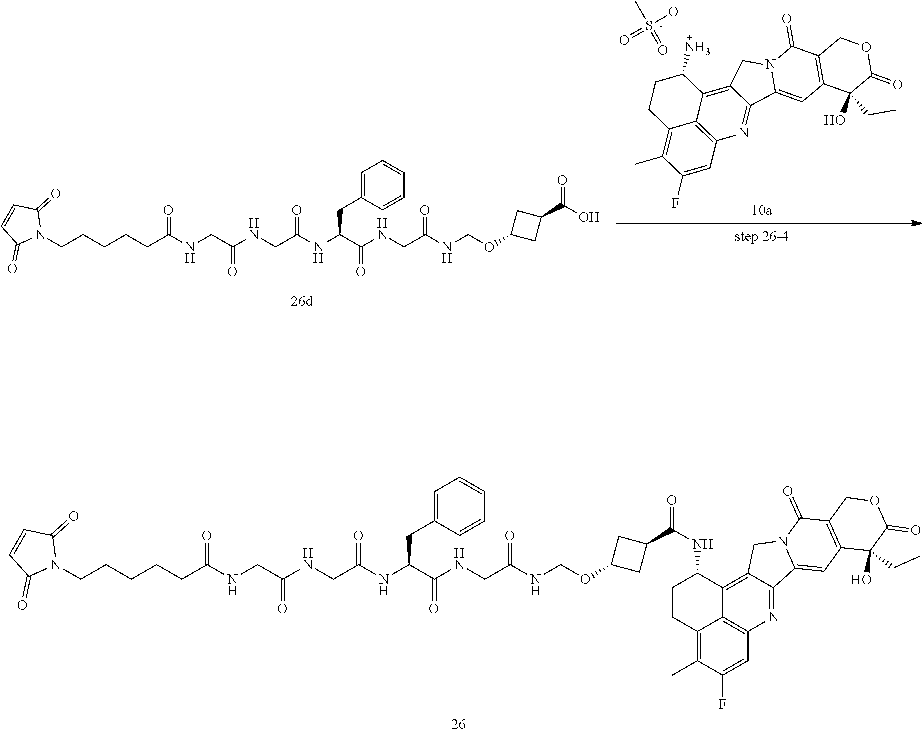

### Step 1

### Methyl (1S,3r)-3-(((S)-11-benzyl-1-(9H-fluoren-9-yl)-3,6,9,12,15-pentaoxo-2-oxa-4,7,10,13,16-pentaazaheptadecan-17-yl)oxy)cyclobutane-1-carboxylate (26b)

Compound 26b (105 mg, 94.5% yield) was synthesized according to synthetic procedure of step 1 of example 25.

MS (ESI) m/z: 722.5 [M+Na+]

### Step 2

### (1S,3R)-3-(((S)-13-amino-7-benzyl-3,6,9,12-tetraoxo-2,5,8,11-tetraazatridecyl)oxy) cyclobuten e-1-carboxylic acid (26c)

To a solution of 26b (105 mg, 0.15 mmol) in MeOH (1 mL) and H2O (0.2 mL) was added K2CO3 (414.76 mg, 3.0 mmol). The resulted white suspension was reacted at r.t. for 3 h. The white suspension was acidified with 1N HCl to pH=6 and the mixture was freeze-dried to provide 26c (485 mg, crude).

MS (ESI) m/z: 486.4 [M+Na+]

### Step 3

### (1S,3r)-3-(((S)-7-benzyl-20-(2,5-dioxo-2,5-dihydro-1H-pyrrol-1-yl)-3,6,9,12,15-pentaoxo-2,5,8,11,14-pentaazaicosyl)oxy)cyclobutane-1-carboxylic acid (26d)

Compound 26d (23 mg, 24% yield) was synthesized according to synthetic procedure of step 8 of example 2.

MS (ESI) m/z: 655.4 [M−H]−

### Step 4

### (1S,3S)-3-(((S)-7-benzyl-20-(2,5-dioxo-2,5-dihydro-1H-pyrrol-1-yl)-3,6,9,12,15-pentaoxo-2,5,8,11,14-pentaazaicosyl)oxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)cyclobutane-1-carboxamide (26)

Compound 26 (20.5 mg, 54.5% yield) was synthesized according to synthetic procedure of step 3 of example 25.

MS (ESI) m/z: 1074.9 [M+H]+

### Example 27

[chemical structure]

### Step 1

### (5S,8S)-1-(9H-fluoren-9-yl)-5-isopropyl-3,6,9-trioxo-8-(3-ureidopropyl)-2-oxa-4,7,10-triazaundecan-1-yl acetate (27b)

27b (585 mg, 51.8% yield) was synthesized according to the synthetic procedure of step 5 of example 22.

MS (ESI) m/z: 590.6 [M+H]+

### Step 2

### Benzyl (5S,8S)-1-(9H-fluoren-9-yl)-5-isopropyl-3,6,9-trioxo-8-(3-ureidopropyl)-2,12-dioxa-4,7,10-triazatetradecan-14-oate (27d)

To the solution of 27b (90 mg, 0.16 mmol) and benzyl 2-hydroxyacetate (132 mg, 0.79 mmol) in dry THF (5 mL) was added dried 4 Å molecular sieves (600 mg), stirred at r.t. for 30 min. To the solution was added scandium trifluoromethanesulfonate (94 mg, 0.19 mmol), stirred at r.t. for 6 h. The solution was filtered through Celite, concentrated and purified by flash column chromatography (DCM/MeOH=10/1) to give the 27d (82 mg, 76.1% yield) as a white solid.

MS (ESI) m/z: 696.7 [M+Na]+

### Step 3

### (5S,8S)-1-(9H-fluoren-9-yl)-5-isopropyl-3,6,9-trioxo-8-(3-ureidopropyl)-2,12-dioxa-4,7,10-triazatetradecan-14-oic acid (27e)

To a solution of 27d (151 mg, 0.22 mmol) in MeOH (3 mL) was added 10% Pd/C (35 mg) and the suspension was purged with H2 balloon for three times then stirred at r.t. for 90 min under H2 pressure. On completion of the reaction, Pd/C was filtered off and methanol was removed under reduced pressure to provide 27e (125 mg, 96% yield).

MS (ESI) m/z: 606.4 [M+Na]+

### Step 4

### (S)-2-((S)-2-amino-3-methylbutanamido)-N-((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)-5-ureidopentanamide (27f)

To a mixture of 27e (50 mg, 0.086 mmol) and TSTU (25.85 mg, 0.086 mmol) in DMF (2 mL) was added DIPEA (33.216 mg, 0.257 mmol). The mixture was reacted at r.t. for 10 min and the acid was converted to active ester based on LCMS. 10a (45.67 mg, 0.086 mmol) was added and reacted at the same temperature for another 5 hr. On completion of the reaction, piperidine (72.30 mg, 0.849 mmol) was added and the mixture was left at r.t. for another 10 min. The mixture was filtered and the filtrate was purified using prep-HPLC (Method: column: XBridge Prep C18 OBD Sum 19*250 mm; Mobile phase: A—water (0.1% formic acid): B—acetonitrile; Flow rate: 20 mL/min) to provide 27f (43 mg, 65.0% yield).

MS (ESI) m/z: 779.6 [M+H]+

### Step 5

### Methyl (3S,4R,5R)-5-((7S,10S,13S)-13-((((9H-fluoren-9-yl)methoxy)carbonyl)amino)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10-isopropyl-1,6,9,12-tetraoxo-7-(3-ureidopropyl)-3-oxa-5,8,11-triazahexadecan-16-amido)-3,4-dihydroxytetrahydrofuran-2-carboxylate (27 g)

HATU (30.21 mg, 0.079 mmol) and DIPEA (30.80 mg, 0.238 mmol) were added to a solution of 5 (42 mg, 0.079 mmol) in DMF (2 mL). The reaction mixture was reacted at r.t. for 10 min. Then added 27f (30.94 mg, 0.040 mmol) left at the same temperature for another 1 h. The mixture was filtered and the filtrate was purified using prep-HPLC (Method: column: XBridge Prep C18 OBD 5 μm 19*250 mm; Mobile phase: A—water (0.1% formic acid): B—acetonitrile; Flow rate: 20 mL/min) to afford 27 g (21 mg, 41% yield).

MS (ESI) m/z: 1311.8 [M+Na]+

### Step 6

### (3S,4R,5R)-5-((7S,10S,13S)-13-amino-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10-isopropyl-1,6,9,12-tetraoxo-7-(3-ureidopropyl)-3-oxa-5,8,11-triazahexadecan-16-amido)-3,4-dihydroxytetrahydrofuran-2-carboxylic acid (27 h)

To a solution of 27 g (21.0 mg, 0.016 mmol) in THE (2 mL) was added Lithium hydroxide aqueous solution (1N, 0.977 mL, 0.977 mmol). The mixture was stirred at r.t. for 2 h. On completion of the reaction, the mixture was acidified by 1N HCl to pH=6, filtered and the filtrate was purified using prep-HPLC (Method: column: XBridge Prep C18 OBD Sum 19*250 mm; Mobile phase: A—water (0.1% formic acid): B—acetonitrile; Flow rate: 20 mL/min) to afford 27 h (11.1 mg, 64.7% yield).

MS (ESI) m/z: 1053.7 [M+H]+

### Step 7

### (3S,4R,5R)-5-((7S,10S,13S)-13-(6-(2,5-dioxo-2,5-dihydro-1H-pyrrol-1-yl)hexanamido)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-10-isopropyl-1,6,9,12-tetraoxo-7-(3-ureidopropyl)-3-oxa-5,8,11-triazahexadecan-16-amido)-3,4-dihydroxytetrahydrofuran-2-carboxylic acid (27)

Compound 27 (5.7 mg, 48% yield) was synthesized according to synthetic procedure of step 8 of example 2.

### Example 28

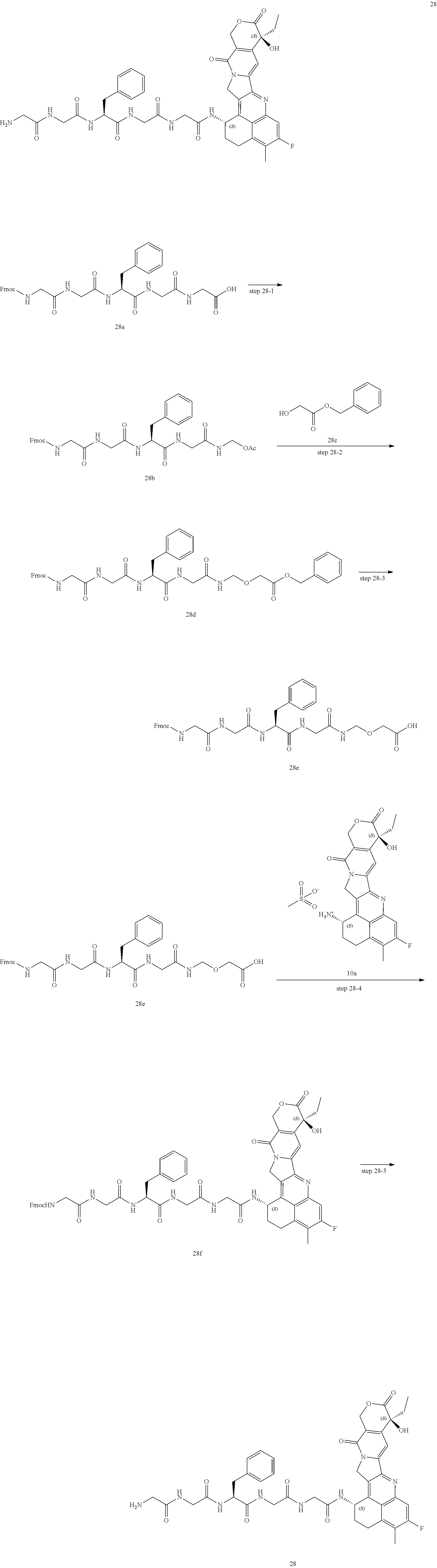

### Step 1

### (S)-1-benzyl-1-(9H-fluoren-9-yl)-3,6,9,12,15-pentaoxo-2-oxa-4,7,10,13,16-pentaazaheptadecan-1′7-yl acetate (28b)

To a solution of 28a (3.0 g, 4.873 mmol) in DMF (26 mL) were added catalytic Cu(OAc)2 (336.33 mmol), Pb(OAc)4 (2.46 g, 5.55 mmol) and HOAc (632.62 uL, 11.062 mmol). The resulted blue mixture was purged with N2 for three times and stirred at 60° C. for 70 min. On completion of the reaction, the mixture cooled to room temperature and quenched with water (100 mL), extracted with EtOAc (100 mL*3). After separation the combined organic layers were washed with brine (150 mL*3), dried over Na2SO4, filtered and the filtrate was concentrated under vacuum to give a residue. It was purified by silica gel column flash chromatography in 10% methanol in Dichloromethane as eluent to provide 28b (3.0 g, 97.8% yield)

MS (ESI) m/z: 652.5 [M+Na]+

### Step 2

### Benzyl (S)-11-benzyl-1-(9H-fluoren-9-yl)-3,6,9,12,15-pentaoxo-2,18-dioxa-4,7,10,13,16-pentaazaicosan-20-oate (28d)

Compound 28c (995 mg, 77.400 yield) was synthesized according to synthetic procedure of step 1 of example 25.

MS (ESI) m/z: 758.5 [M+Na+]

### Step 3

### (S)-11-benzyl-1-(9H-fluoren-9-yl)-3,6,9,12,15-pentaoxo-2,18-dioxa-4,7,10,13,16-pentaazaicosan-20-oic acid (28e)

To a solution of (995 mg, 1.352 mmol) in MeOH (5 mL) was added 10% Pd/C (100 mg) and the suspension was purged with H2 balloon for three times then stirred at r.t. for 90 min under H2 pressure. On completion of the reaction, Pd/C was filtered off and methanol was removed under reduced pressure to provide 28e (770 mg, 88.2% yield).

MS (ESI) m/z: 668.5 [M+Na]+

### Step 4

### (9H-fluoren-9-yl)methyl ((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)carbamate (28f)

HATU (453.447 mg, 1.193 mmol) and DIPEA (462.38 mg, 3.578 mmol) were added to a solution of 28e (770 mg, 1.193 mmol) in DMF (10 mL). The reaction mixture was reacted at r.t. for 10 min. Then added 10a (639.3 mg, 1.204 mmol) left at the same temperature for another 1 h. The mixture was diluted with EtOAc (50 mL), washed with brine (30 mL*3). The organic layers was dried over Na2SO4, filtered and the filtrate was concentrated under vacuum to give 28f (1.27 g, crude)

MS (ESI) m/z: 1085.7 [M+Na]+

### Step 5

### (S)-2-(2-(2-aminoacetamido)acetamido)-N-(2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino [1,2-b]quinolin-1-yl)amino)-2-oxoethoxy)methyl)amino)-2-oxoethyl)-3-phenylpropanamide (28)

To a mixture of 28f (1.27 g crude) in THE (20 mL) was added Et2N (1.74 g, 23.855 mmol). The mixture was reacted at r.t. for 2 h. On completion of the reaction, the mixture was filtered and the filtrate was purified by prep-HPLC (Method: column: XBridge Prep C18 OBD Sum 19*250 mm; Mobile phase: A—water (0.1% formic acid): B—acetonitrile; Flow rate: 20 mL/min) to provide 28 (296 mg, 29.5% yield).

MS (ESI) m/z: 841.6 [M+H]+

### Example 29

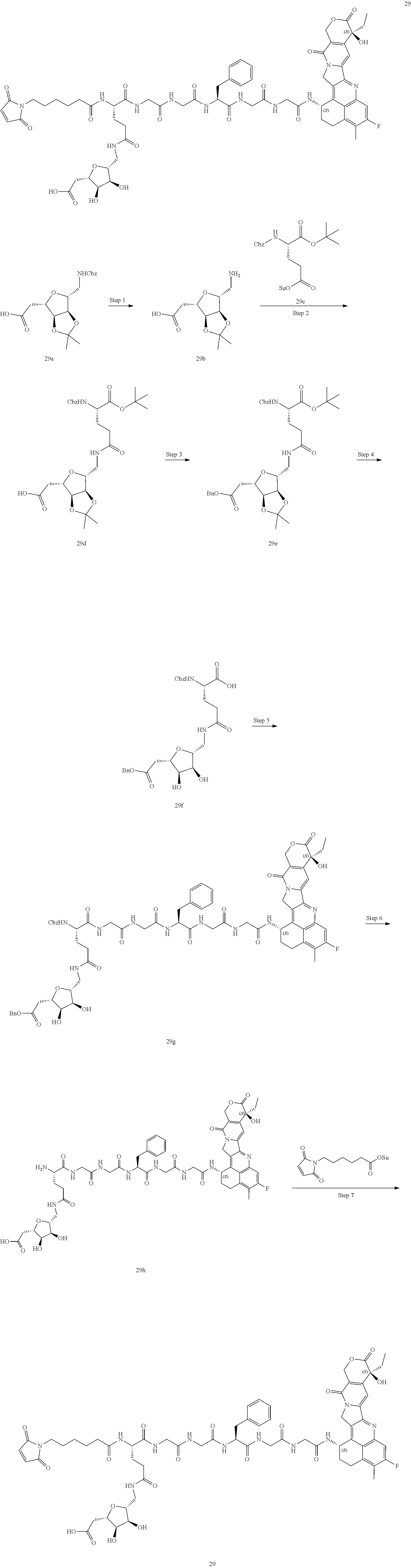

### Step 1

### 2-((3aS,4S,6R,6aR)-6-(aminomethyl)-2,2-dimethyltetrahydrofuro[3,4-d][1,3]dioxol-4-yl)acetic acid (29b)

29a (purchased form WuXi apptec, 450 mg, 1.23 mmol) was dissolved in THE (9 mL) and MeOH (9 mL) followed by the addition of Pd/C (wet, 10%, 110 mg). The resulting mixture was stirred at 25° C. for 2 h. After complete reaction, the reaction mixture was concentrated under reduced pressure to afford 29b (284 mg, quant.) as a clear oil which was used directly in next step without purification.

MS (ESI) m/z: 230.2 [M−H]−

### Step 2

### 2-((3aS,4S,6R,6aR)-6-(((S)-4-(((benzyloxy)carbonyl)amino)-5-(tert-butoxy)-5-oxopentanamido)methyl)-2,2-dimethyltetrahydrofuro[3,4-d][1,3]dioxol-4-yl)acetic acid (29d)

29d (520 mg, 76.8% yield) was synthesized from 29b and 29c (commercially available) according to the synthetic procedure of step 2 of example 1.

MS (ESI) m/z: 573.5 [M+Na]+

### Step 3

### Tert-butyl N5-(((3aR,4R,6S,6aS)-6-(2-(benzyloxy)-2-oxoethyl)-2,2-dimethyltetrahydrofuro[3,4-d][1,3]dioxol-4-yl)methyl)-N2-((benzyloxy)carbonyl)-L-glutaminate (29e)

29e (573 mg, 80.5% yield) was synthesized according to the synthetic procedure of step 3 of example 22.

MS (ESI) m/z: 663.5 [M+Na]+

### Step 4

### N5-(((2R,3S,4R,5S)-5-(2-(benzyloxy)-2-oxoethyl)-3,4-dihydroxytetrahydrofuran-2-yl)methyl)-N2-((benzyloxy)carbonyl)-L-glutamine (29f)

29e (150 mg, 0.23 mmol) was dissolved in DCM (2 mL) followed by the addition of a mixed solvent of TFA/H2O (v/v=9:1, 2 mL) at 0° C. The mixture was firstly stirred at 0° C. for 1.5 h and then further stirred at 20° C. for 1 h. After complete reaction, the reaction mixture was concentrated under reduced pressure to afford a pale yellow oil as the crude product which was purified by prep-HPLC (Method: column: XBridge Prep C18 OBD Sum 19*250 mm; Mobile phase: A—water (0.1% formic acid): B—acetonitrile; Flow rate: 20 mL/min) to afford 29f (27 mg, 21.2% yield) as a white solid.

MS (ESI) m/z: 545.5 [M+H]+

### Step 5

### Benzyl 2-((2S,3R,4S,5R)-5-((6S,15S)-15-benzyl-6-(((benzyloxy)carbonyl)amino)-24-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,24-heptaoxo-22-oxa-2,8,11,14,17,20-hexaazatetracosyl)-3,4-dihydroxytetrahydrofuran-2-yl)acetate (29 g)

29 g (26 mg, 41.4% yield) was synthesized according to the synthetic procedure of step 8 of example 22.

MS (ESI) m/z: 1389.7 [M+Na]+

### Step 6

### 2-((2S,3R,4S,5R)-5-((6S,15S)-6-amino-15-benzyl-24-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,24-heptaoxo-22-oxa-2,8,11,14,17,20-hexaazatetracosyl)-3,4-dihydroxytetrahydrofuran-2-yl)acetic acid (29 h)

29 h (22 mg, crude) was synthesized according to the synthetic procedure of step 7 of example 22.

MS (ESI) m/z: 1143.7 [M+H]+

### Step 7

### 2-((2S,3R,4S,5R)-5-((6S,15S)-6-amino-15-benzyl-24-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,24-heptaoxo-22-oxa-2,8,11,14,17,20-hexaazatetracosyl)-3,4-dihydroxytetrahydrofuran-2-yl)acetic acid (29)

29 (4.5 mg, 77.0% yield) was synthesized according to the synthetic procedure of step 10 of example 22.

MS (ESI) m/z: 1358.8 [M+Na]+

### Example 30

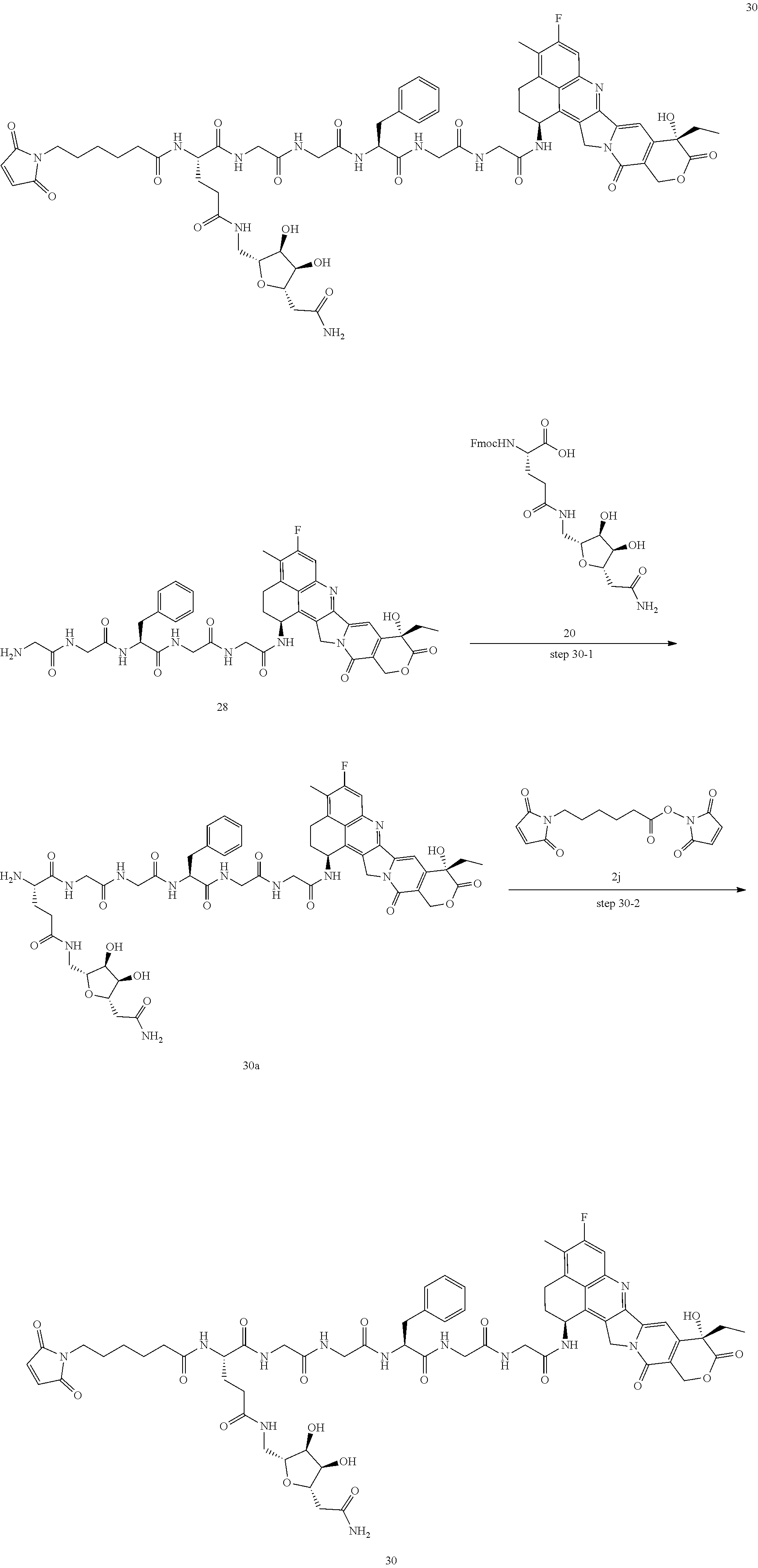

### Step 1

### (S)-2-amino-N5-(((2R,3S,4R,5S)-5-(2-amino-2-oxoethyl)-3,4-dihydroxytetrahydrofuran-2-yl)methyl)-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)pentanediamide (30a)

To a mixture of 20 (20 mg, 0.037 mmol) and HATU (14.05 mg, 0.037 mmol) in DMF (1 mL) was added DIPEA (14.33 mg, 0.111 mmol). The mixture was reacted at r.t. for 10 min. 28 (40.0 mg, 0.048 mmol) was added and reacted at the same temperature for another 15 min. On completion of the reaction, piperidine (139.34 mg, 0.366 mmol) was added and the mixture was left at r.t. for another 10 min. The mixture was filtered and the filtrate was purified using prep-HPLC (Method: column: XBridge Prep C18 OBD 5 μm 19*250 mm; Mobile phase: A—water (0.1% formic acid): B—acetonitrile; Flow rate: 20 mL/min) to provide 30a (30.0 mg, 71.7% yield).

MS (ESI) m/z: 1164.8 [M+Na]+

### Step 2

### (S)—N5-(((2R,3S,4R,5S)-5-(2-amino-2-oxoethyl)-3,4-dihydroxytetrahydrofuran-2-yl)methyl)-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)pentanediamide (30)

Compound 30 (7.5 mg, 42.8% yield) was synthesized according to synthetic procedure of step 8 of example 2.

MS (ESI) m/z: 1335.9 [M+H]+

### Example 31

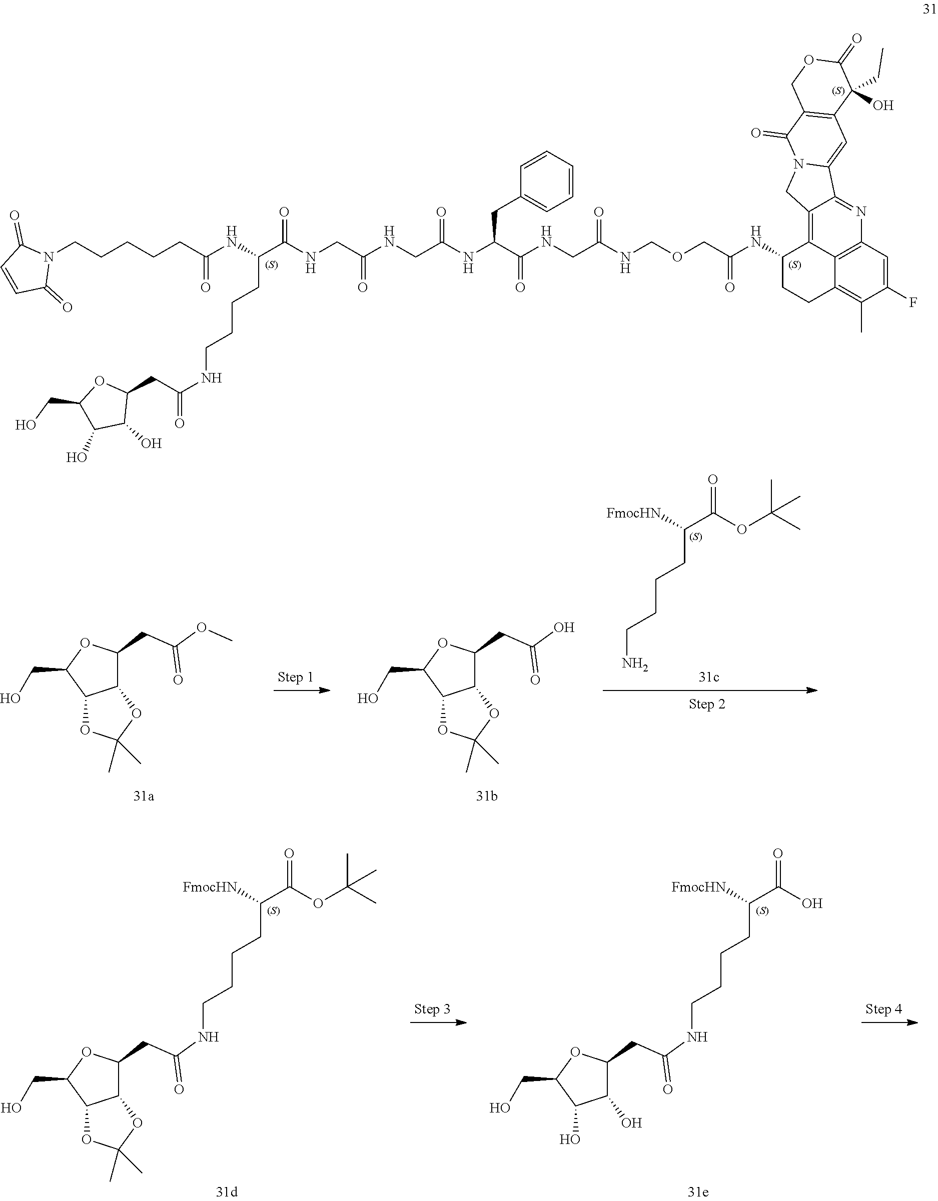

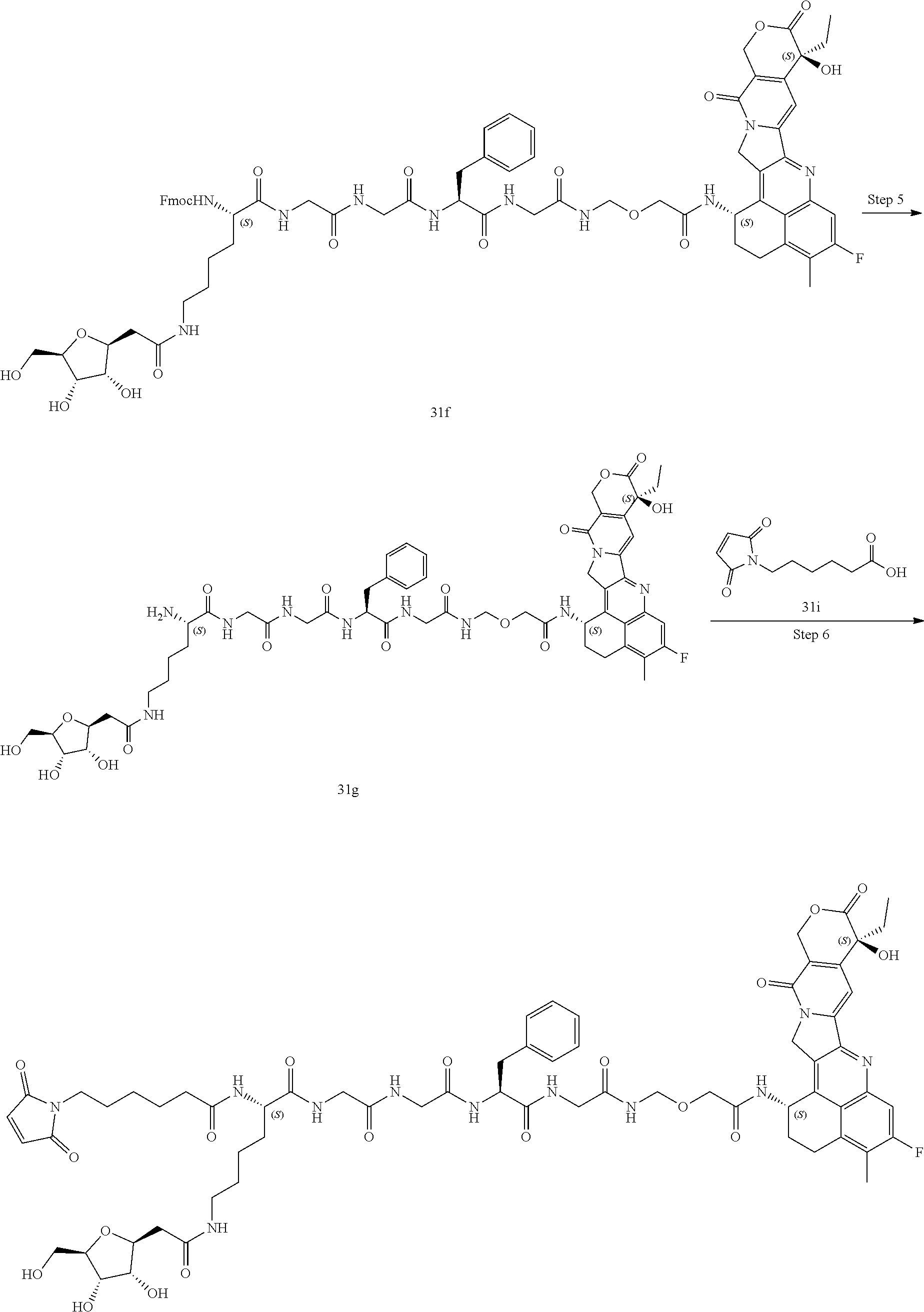

### Step 1

### 2-((3aS,4S,6R,6aR)-6-(hydroxymethyl)-2,2-dimethyltetrahydrofuro[3,4-d][1,3]dioxol-4-yl)acetic acid (31b)

31b (231 mg, crude, 87.1% yield) was synthesized according to the synthetic procedure of step 2 of example 22.

### Step 2

### Tert-butyl N2-(((9H-fluoren-9-yl)methoxy)carbonyl)-N6-(2-((3aS,4S,6R,6aR)-6-(hydroxymethyl)-2,2-dimethyltetrahydrofuro[3,4-d][1,3]dioxol-4-yl)acetyl)-L-lysinate (31d)

31d (393 mg, 64.9% yield) was synthesized according to the synthetic procedure of step 8 of example 22.

MS (ESI) m/z: 639.6 [M+H]+

### Step 3

### N2-(((9H-fluoren-9-yl)methoxy)carbonyl)-N6-(2-((2S,3R,4S,5R)-3,4-dihydroxy-5-(hydroxymethyl)tetrahydrofuran-2-yl)acetyl)-L-lysine (31e)

31e (27 mg, 63.5% yield) was synthesized according to the synthetic procedure of step 4 of example 29.

MS (ESI) m/z: 543.5 [M+H]+

### Step 4

### (9H-fluoren-9-yl)methyl ((10S,19S)-10-benzyl-26-((2S,3R,4S,5R)-3,4-dihydroxy-5-(hydroxymethyl)tetrahydrofuran-2-yl)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15,18,25-heptaoxo-3-oxa-5,8,11,14,17,24-hexaazahexacosan-19-yl)carbamate (31f)

31f (40 mg, 51.3% yield) was synthesized according to the synthetic procedure of step 8 of example 22.

MS (ESI) m/z: 1387.9 [M+Na]+

### Step 5

### (S)-2-amino-N—((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-6-(2-((2S,3R,4S,5R)-3,4-dihydroxy-5-(hydroxymethyl)tetrahydrofuran-2-yl)acetamido)hexanamide (31 g)

31f (20 mg, 0.015 mmol) was dissolved in DMF (1 mL) followed by the addition of Et2NH (15 μL, 0.15 mmol). The resulting mixture was stirred at 25° C. for 2 h. After complete reaction, the reaction mixture was concentrated under reduced pressure to afford 31 g (19 mg, quant.) as a grey solid which was used directly in next step without purification.

MS (ESI) m/z: 1143.8 [M+H]+

### Step 6

### (S)—N—((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-6-(2-((2S,3R,4S,5R)-3,4-dihydroxy-5-(hydroxymethyl)tetrahydrofuran-2-yl)acetamido)-2-(6-(2,5-dioxo-2,5-dihydro-1H-pyrrol-1-yl)hexanamido)hexanamide (31)

31 (13.9 mg, 70.4% yield) was synthesized according to the synthetic procedure of step 8 of example 22.

MS (ESI) m/z: 1358.9 [M+Na]+

### Example 32

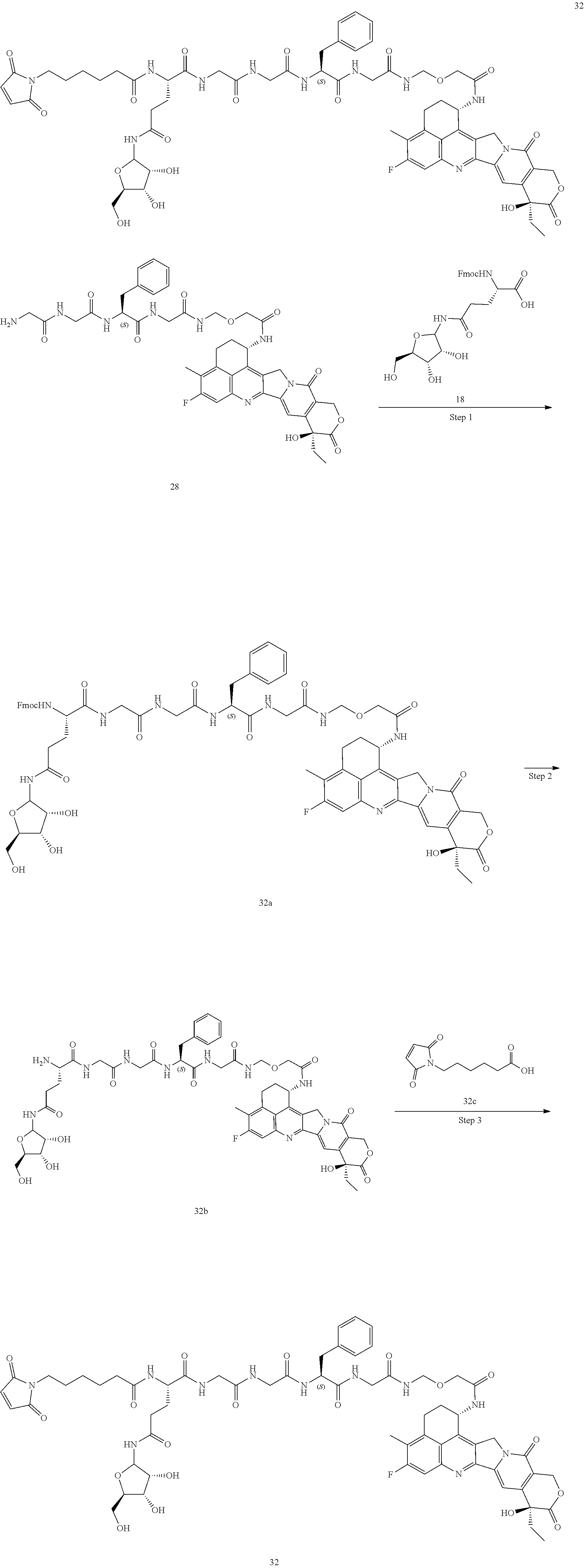

### Step 1

### (9H-fluoren-9-yl)methyl ((10S,19S)-10-benzyl-22-(((3R,4S,5R)-3,4-dihydroxy-5-(hydroxymethyl)tetrahydrofuran-2-yl)amino)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15,18,22-heptaoxo-3-oxa-5,8,11,14,17-pentaazadocosan-19-yl)carbamate (32a)

To the solution of 18 (40 mg, 0.08 mmol), 28 (60 mg, 0.07 mmol) and HATU (30 mg, 0.08 mmol) in dry DMF (1 mL) was added DIEA (36 μL, 0.21 mmol), stirred at r.t. for 1.5 h. The reaction solution was filtered and purified by prep-HPLC (0.1% FA in water/MeCN), lyophilized to give the title compound 32a (52 mg, 55.1% yield) as a beige solid.

MS (ESI) m/z: 1345.8 [M+Na]+

### Step 2

### (2S)-2-amino-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-((3R,4S,5R)-3,4-dihydroxy-5-(hydroxymethyl)tetrahydrofuran-2-yl)pentanediamide (32b)

To the solution of 32a (52 mg, 0.04 mmol) in DMF (1 mL) was added Et2NH (81 μL, 0.79 mmol), stirred at r.t. for 1 h. The solution was extracted with petroleum ether (3 mL*4), the DMF phase was concentrated to give the title compound 32b (44.4 mg, 102.6%) as a beige solid.

MS (ESI) m/z: 1101.8 [M+H]+

### Step 3

### (2S)—N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-((3R,4S,5R)-3,4-dihydroxy-5-(hydroxymethyl)tetrahydrofuran-2-yl)-2-(6-(2,5-dioxo-2,5-dihydro-1H-pyrrol-1-yl)hexanamido)pentanediamide (32)

To the solution of 32b (25 mg, 22.7 mol), 6-(2,5-dioxo-2,5-dihydro-1H-pyrrol-1-yl)hexanoic acid 32c (5.3 mg, 25.0 mol) and HATU (9.5 mg, 25.0 mol) in dry DMF (1 mL) was added DIEA (8 μL, 45.4 mol), stirred at r.t. for 20 min. The solution was filtered and purified by prep-HPLC (0.1% FA in water/MeCN), lyophilized to give the title compound 32 (10 mg, 34.0% yield) as a white solid.

MS (ESI) m/z: 1316.8 [M+Na]+

### Example 33

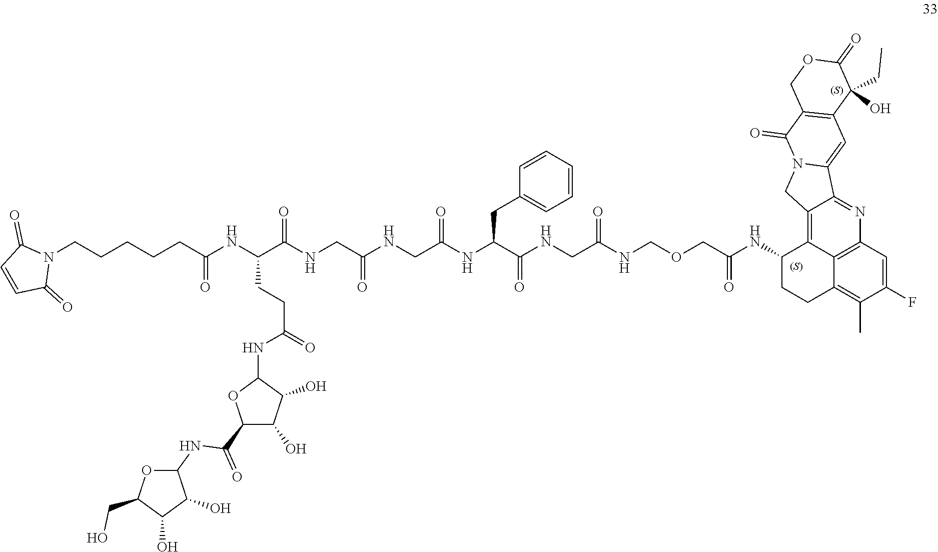

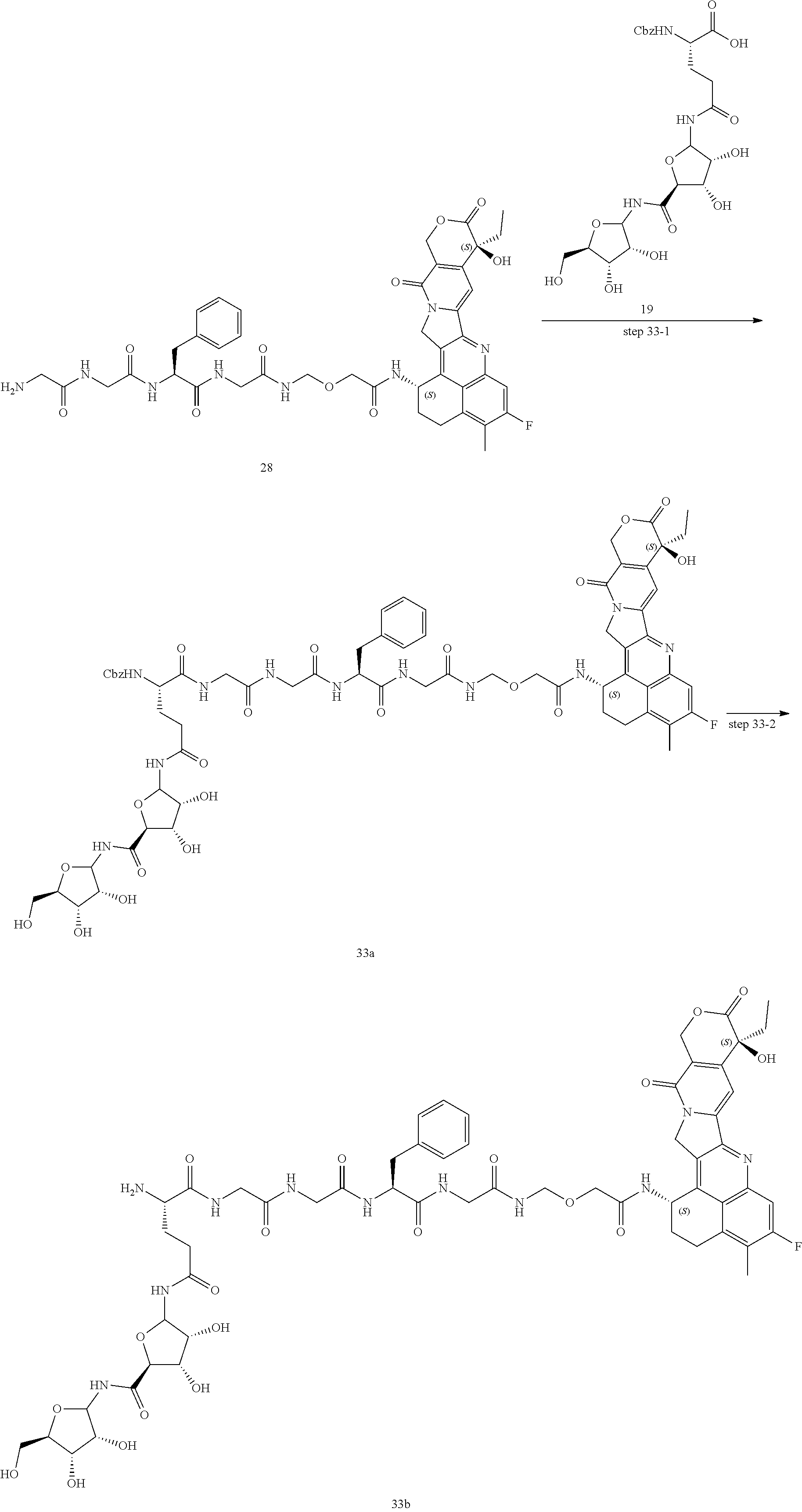

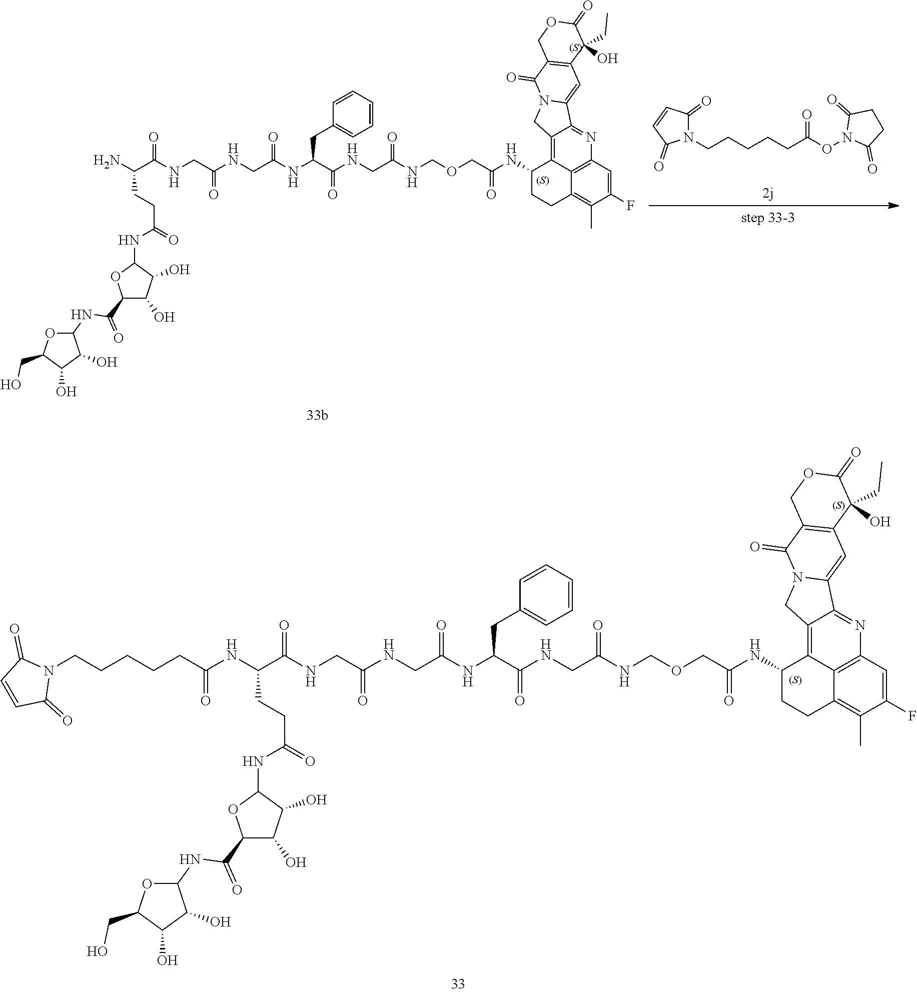

### Step 33-1

### Benzyl ((10S,19S)-10-benzyl-22-(((3R,4S,5S)-5-(((3R,4S,5R)-3,4-dihydroxy-5-(hydroxymethyl) tetrahydrofuran-2-yl)carbamoyl)-3,4-dihydroxytetrahydrofuran-2-yl)amino)-1-(((1S,9S)-9-ethyl-5-fluoro-9-hydroxy-4-methyl-10,13-dioxo-2,3,9,10,13,15-hexahydro-1H,12H-benzo[de]pyrano [3′,4′:6,7]indolizino[1,2-b]quinolin-1-yl)amino)-1,6,9,12,15,18,22-heptaoxo-3-oxa-5,8,11,14,17-pentaazadocosan-19-yl)carbamate (33a)

To a mixture of 19 (26 mg, 0.047 mmol) and HATU (17.73 mg, 0.047 mmol) in DMF (1 mL) was added DIPEA (18.08 mg, 0.14 mmol). The mixture was reacted at r.t. for 10 min. 28 (39.21 mg, 0.047 mmol) was added and reacted at the same temperature for another 15 min. On completion of the reaction, The mixture was filtered and the filtrate was purified using prep-HPLC (Method: column: XBridge Prep C18 OBD Sum 19*250 mm; Mobile phase: A—water (0.1% formic acid): B—acetonitrile; Flow rate: 20 mL/min) to provide 33a (35.0 mg, 54.4% yield).

MS (ESI) m/z: 1403.9 [M+Na]+

### Step 33-2

### (2S)-2-amino-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-((3R,4S,5S)-5-(((3R,4S,5R)-3,4-dihydroxy-5-(hydroxymethyl)tetrahydrofuran-2-yl)carbamoyl)-3,4-dihydroxytetrahydrofuran-2-yl)pentanediamide (33b)

To a solution of 33a (35 mg, 0.025 mmol) in MeOH (1 mL) was added 10% Pd/C (5.3 mg) and the suspension was purged with H2 balloon for three times then stirred at r.t. for 4 h under H2 pressure. On completion of the reaction, Pd/C was filtered off and methanol was removed under reduced pressure to provide 33b (28.3 mg, 89.6% yield).

MS (ESI) m/z: 1246.8 [M+H]+

### Step 33-3

### (2S)—N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-((3R,4S,5S)-5-(((3R,4S,5R)-3,4-dihydroxy-5-(hydroxymethyl)tetrahydrofuran-2-yl)carbamoyl)-3,4-dihydroxytetrahydrofuran-2-yl)-2-(6-(2,5-dioxo-2,5-dihydro-1H-pyrrol-1-yl)hexanamido)pentanediamide (33)

Compound 33 (11.8 mg, 36.1% yield) was synthesized according to synthetic procedure of step 8 of example 2.

MS (ESI) m/z: 1462.9 [M+Na]+

### Example 34

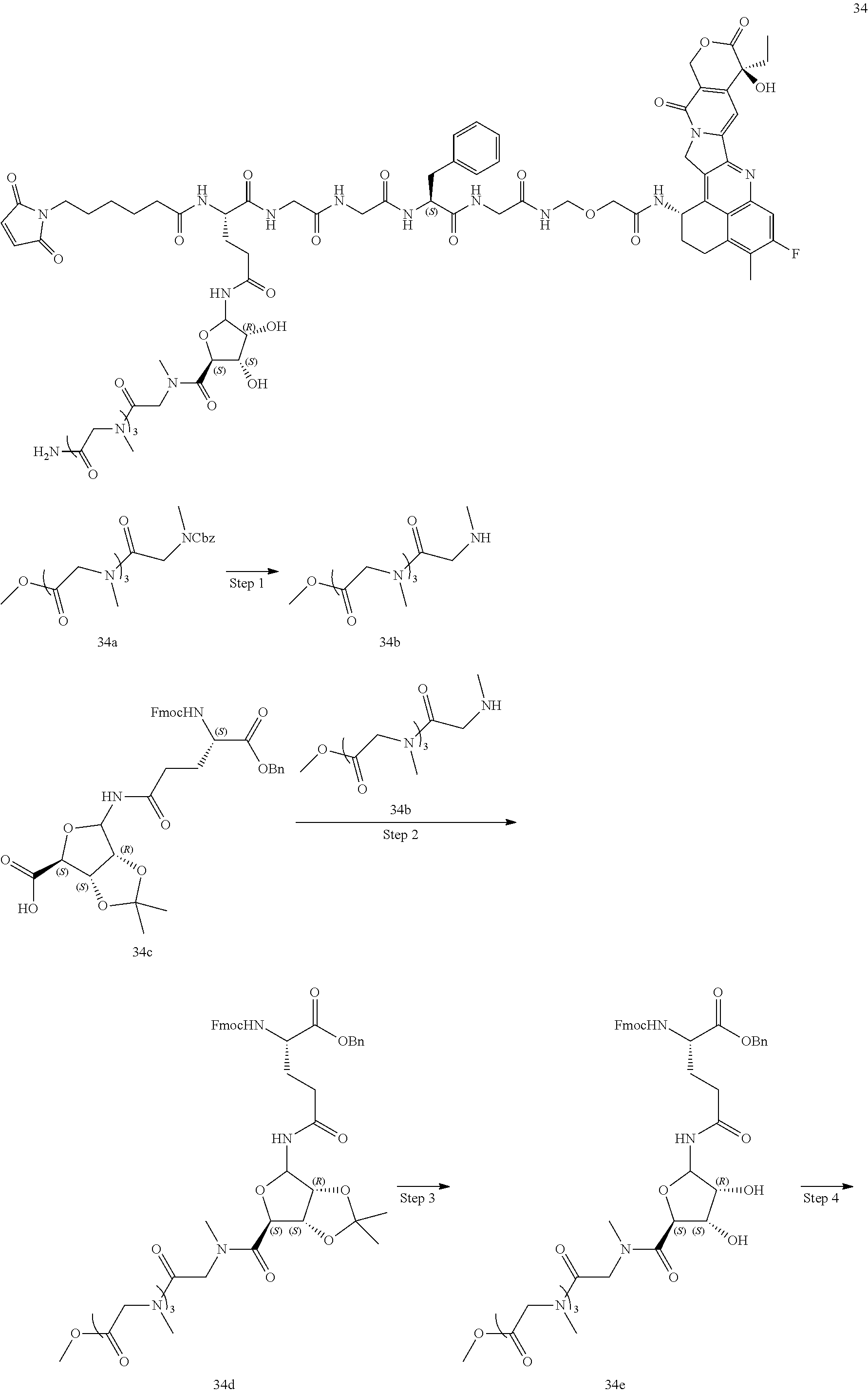

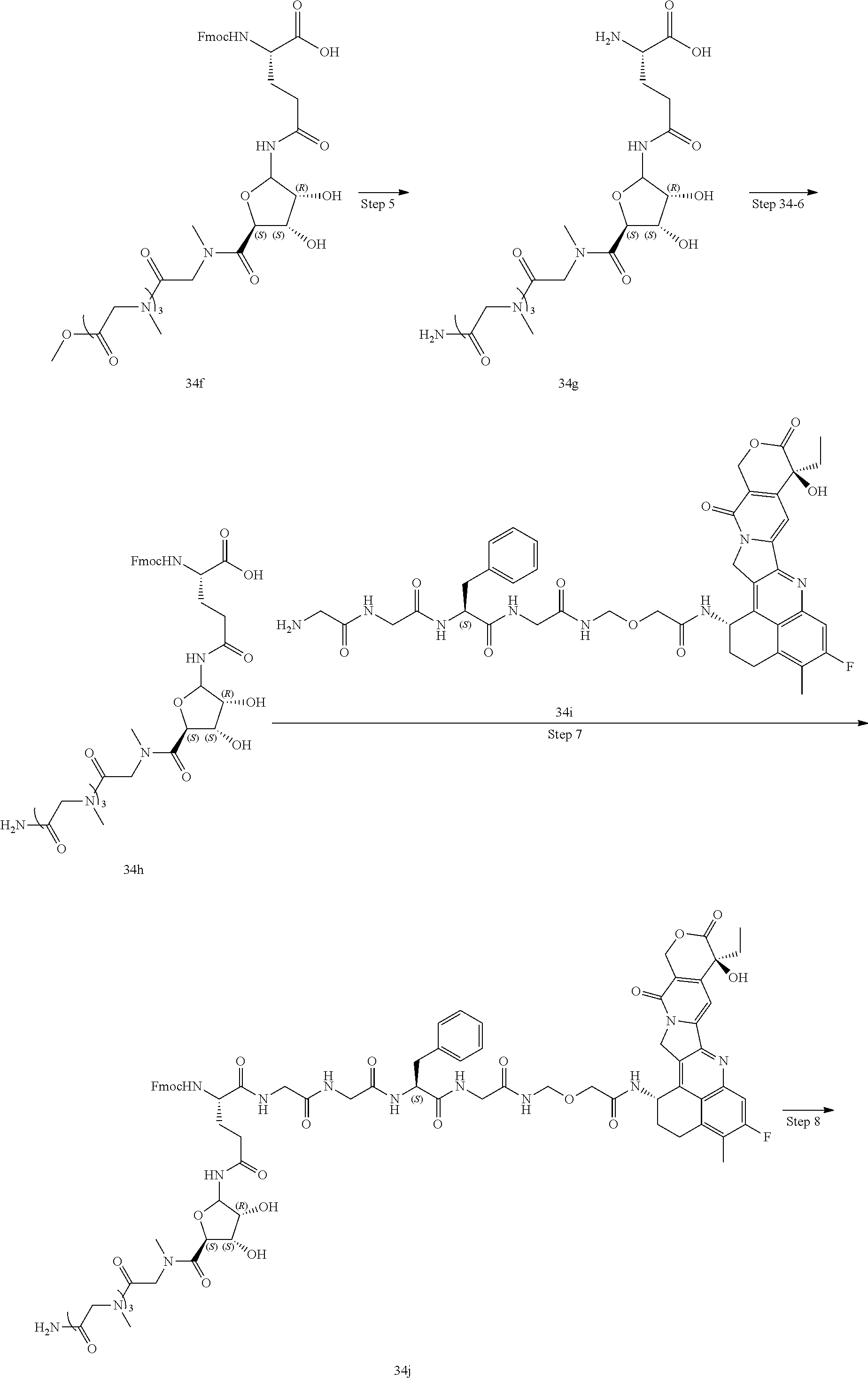

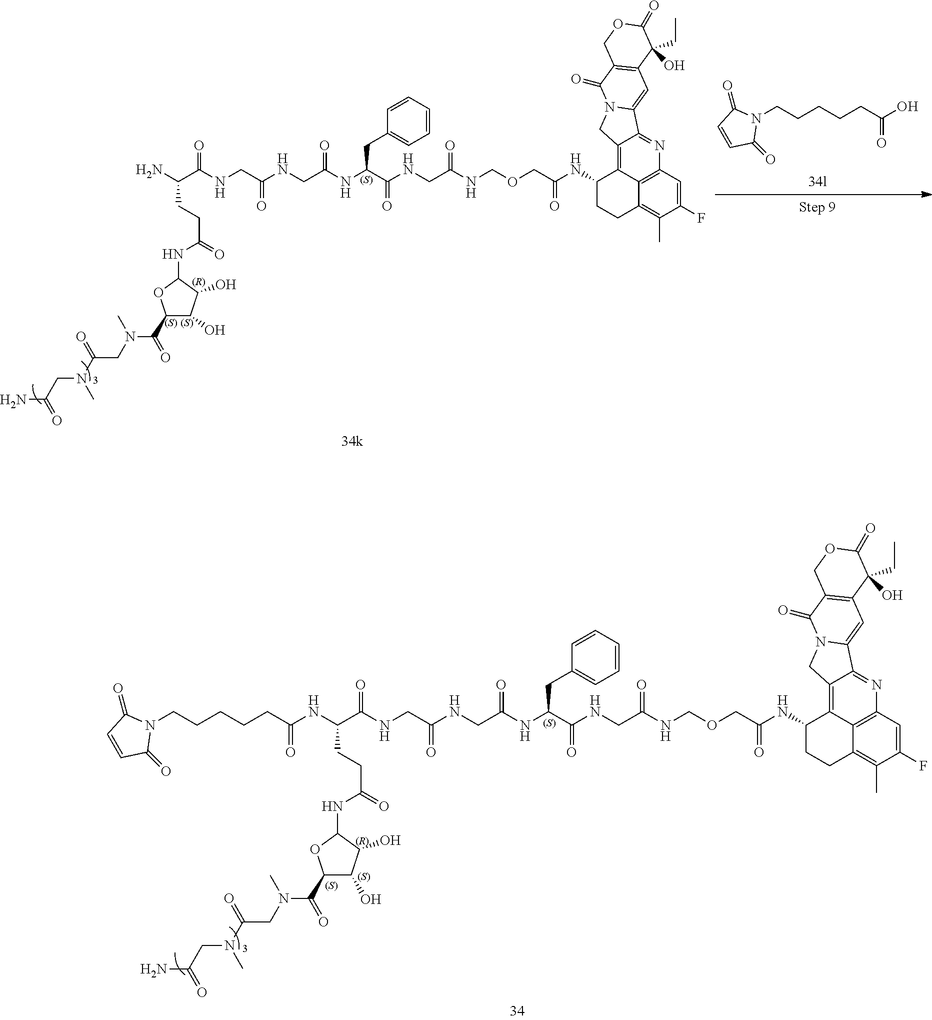

### Step 1

### Methyl N-methyl-N—(N-methyl-N—(N-methyl-N-(2-(methyl-12-azaneyl)acetyl)glycyl)glycyl)glycinate (34b)

To a solution of compound 34a (200 mg, 0.44 mmol) in MeOH (6 mL) was added wet Pd/C (20 mg, 10 wt %). The mixture was stirred at r.t. for 2 h under H2 (15 psi) atmosphere. The mixture was filtered through a pad of celite, concentrated to give the crude product. Compound 34b (180 mg, crude) was obtained as colorless oil.

MS (ESI) m/z: 317.4 [M+H]+.

### Step 2

### Methyl 1-((3aS,6R,6aR)-6-((S)-4-((((9H-fluoren-9-yl)methoxy)carbonyl)amino)-5-(benzyloxy)-5-oxopentanamido)-2,2-dimethyltetrahydrofuro[3,4-d][1,3]dioxol-4-yl)-2,5,8,11-tetramethyl-1,4,7,10-tetraoxo-2,5,8,11-tetraazatridecan-13-oate (34d)

To a solution of compound 34c (368 mg, 0.57 mmol) in pyridine (4 mL) were added 34b (180 mg, 0.63 mmol) and EDCI (130 mg, 0.63 mmol). The mixture was stirred at r.t. for 6 h. The mixture was concentrated and the residue was purified by prep-HPLC (Method: column: XBridge Prep C18 OBD 5 μm 19*250 mm; Mobile phase: A—water (0.1% formic acid): B—acetonitrile; Flow rate: 20 mL/min). Compound 34d (120 mg, 22.3% yield) was obtained as white solid.

MS (ESI) m/z: 943.7 [M+H]+.

### Step 3

### Methyl 1-((3S,4R,5R)-5-((S)-4-((((9H-fluoren-9-yl)methoxy)carbonyl)amino)-5-(benzyloxy)-5-oxopentanamido)-3,4-dihydroxytetrahydrofuran-2-yl)-2,5,8,11-tetramethyl-1,4,7,10-tetraoxo-2,5,8,11-tetraazatridecan-13-oate (34e)

To a solution of compound 34d (120 mg, 0.13 mmol) in THE (1 mL) was added THF-H2O (v:v=4:1, 5 mL). The mixture was stirred at r.t. for 4 h. The mixture was concentrated to give the crude product, which was used for next step without further purification. Compound 34e (115 mg, crude) was obtained as white solid.

MS (ESI) m/z: 903.7 [M+H]+.

### Step 4

### N2-(((9H-fluoren-9-yl)methoxy)carbonyl)-N5-((3R,4S,5S)-3,4-dihydroxy-5-(methyl(5,8,11-trimethyl-3,6,9,12-tetraoxo-2-oxa-5,8,11-triazatridecan-13-yl)carbamoyl)tetrahydrofuran-2-yl)-L-glutamine (34f)

To a solution of compound 34e (115 mg, 0.13 mmol) in MeOH (4 mL) was added wet Pd/C (15 mg, 10%). The mixture was stirred at r.t. under H2 (15 psi) for 5 h. The mixture was filtered through a pad of celite, concentrated. The crude was purified by prep-HPLC (Method: column: XBridge Prep C18 OBD Sum 19*250 mm; Mobile phase: A—water (0.1% formic acid): B—acetonitrile; Flow rate: 20 mL/min). Compound 34f (74 mg, 64.3% yield) was obtained as white solid.

MS (ESI) m/z: 813.6 [M+H]+.

### Step 5

### N5-((3R,4S,5S)-5-((2-((2-((2-((2-amino-2-oxoethyl)(methyl)amino)-2-oxoethyl)(methyl)amino)-2-oxoethyl)(methyl)amino)-2-oxoethyl)(methyl)carbamoyl)-3,4-dihydroxytetrahydrofuran-2-yl)-L-glutamine (34 g)

A solution of compound 34f (74 mg, 0.09 mmol) in NH3-MeOH (6 mL, 7 M) was stirred at 40° C. in a sealed tube for 16 h. The mixture was concentrated, resolved by H2O (10 mL), washed by EA (4*10 mL). The aqueous solution was lyophilized to give the crude product. Compound 34g (68 mg, crude) was obtained as white solid.

MS (ESI) m/z: 576.5 [M+H]+.

### Step 6

### N2-(((9H-fluoren-9-yl)methoxy)carbonyl)-N5-((3R,4S,5S)-5-((2-((2-((2-((2-amino-2-oxoethyl)(methyl)amino)-2-oxoethyl)(methyl)amino)-2-oxoethyl)(methyl)amino)-2-oxoethyl)(methyl)carbamoyl)-3,4-dihydroxytetrahydrofuran-2-yl)-L-glutamine (34 h)

To a solution of compound 34g (68 mg, 0.12 mmol) in CH3CN (3 mL) and H2O (1.5 mL) were added Fmoc-OSu (30.8 mg, 0.09 mmol) and Na2CO3 (18.4 mg, 0.17 mmol). The mixture was stirred at r.t. for 2 h. The mixture was purified by prep-HPLC (Method: column: XBridge Prep C18 OBD Sum 19*250 mm; Mobile phase: A—water (0.1% formic acid): B—acetonitrile; Flow rate: 20 mL/min). Compound 34h (29 mg, 30.2% yield) was obtained as white solid.

MS (ESI) m/z: 798.6 [M+H]+.

### Step 7

### (9H-fluoren-9-yl)methyl ((10S,19S)-22-(((3R,4S,5S)-5-((2-((2-((2-((2-amino-2-oxoethyl)(methyl)amino)-2-oxoethyl)(methyl)amino)-2-oxoethyl)(methyl)amino)-2-oxoethyl)(methyl)carbamoyl)-3,4-dihydroxytetrahydrofuran-2-yl)amino)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15,18,22-heptaoxo-3-oxa-5,8,11,14,17-pentaazadocosan-19-yl)carbamate (34j)

To a solution of compound 34i (29 mg, 0.036 mmol) in DMF (2 mL) were added 34 h (32.1 mg, 0.038 mmol), HATU (20.7 mg, 0.054 mmol) and DIEA (7.1 mg, 0.054 mmol). The mixture was stirred at r.t. for 20 min. The mixture was purified by prep-HPLC (Method: column: XBridge Prep C18 OBD Sum 19*250 mm; Mobile phase: A—water (0.1% formic acid): B—acetonitrile; Flow rate: 20 mL/min). Compound 34j (32 mg, 54.0%) was obtained as white solid.

MS (ESI) m/z: 1644.0 [M+Na]+.

### Step 8

### (S)-2-amino-N5-((3R,4S,5S)-5-((2-((2-((2-((2-amino-2-oxoethyl)(methyl)amino)-2-oxoethyl)(methyl)amino)-2-oxoethyl)(methyl)amino)-2-oxoethyl)(methyl)carbamoyl)-3,4-dihydroxytetrahydrofuran-2-yl)-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)pentanediamide (34k)

To a solution of compound 34j (32 mg, 0.02 mmol) in DMF (2 mL) was added Et2NH (28.9 mg, 0.395 mmol). The mixture was stirred at r.t. for 1 h. The mixture was concentrated and co-evaporated with toluene (3*2 mL). The crude was used for next step without further purification. Compound 34k (34 mg, crude) was obtained as off-white solid.

MS (ESI) m/z: 1400.1 [M+H]+.

### Step 9

### (S)—N5-((3R,4S,5S)-5-((2-((2-((2-((2-amino-2-oxoethyl)(methyl)amino)-2-oxoethyl)(methyl)amino)-2-oxoethyl)(methyl)amino)-2-oxoethyl)(methyl)carbamoyl)-3,4-dihydroxytetrahydrofuran-2-yl)-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)pentanediamide (34)

To a solution of compound 34i (6.3 mg, 0.03 mmol) in DMF (2 mL) were added HATU (11.3 mg, 0.03 mmol) and DIEA (3.8 mg, 0.03 mmol). The mixture was stirred at r.t. for 15 min. Compound 34k (27.6 mg, 0.02 mmol) was added into the mixture. The mixture was stirred at r.t. for 15 min. The mixture was purified by prep-HPLC (Method: column: XBridge Prep C18 OBD Sum 19*250 mm; Mobile phase: A—water (0.1% formic acid): B—acetonitrile; Flow rate: 20 mL/min). Compound 34 (21 mg, 66.8% yield) was obtained as white solid.

MS (ESI) m/z: 1614.1 [M+Na]+.

### Example 35

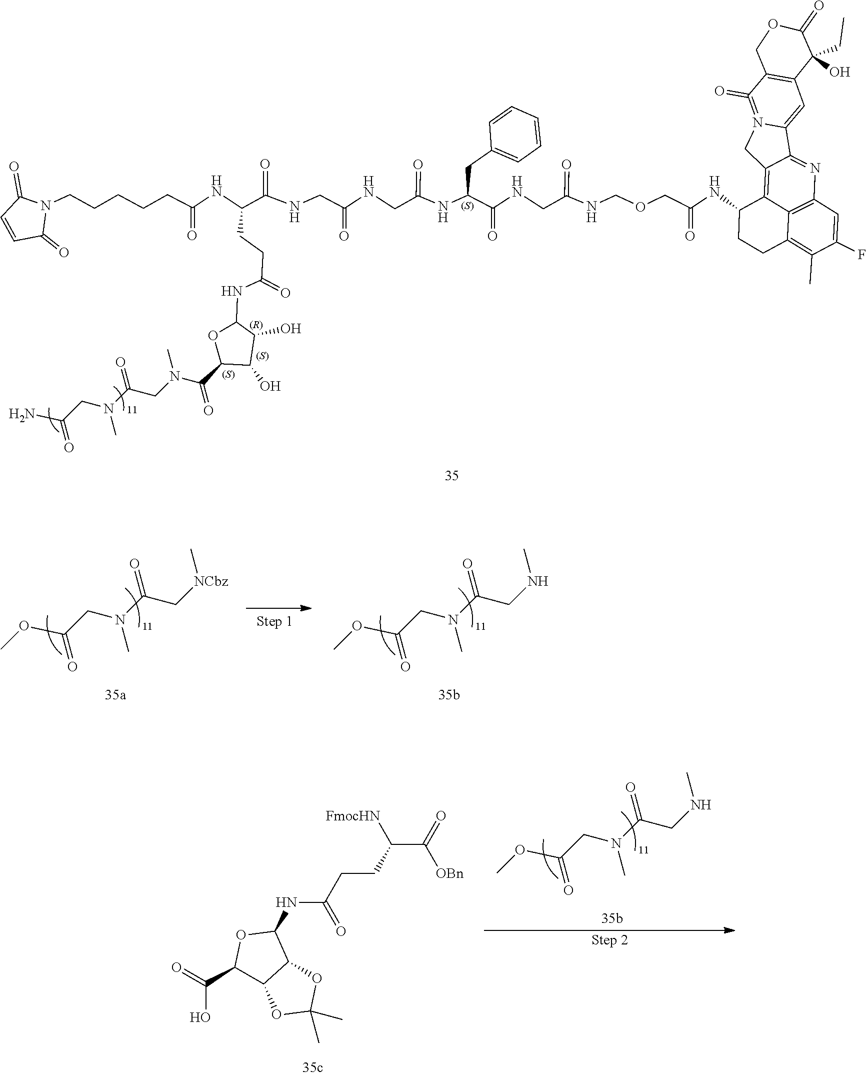

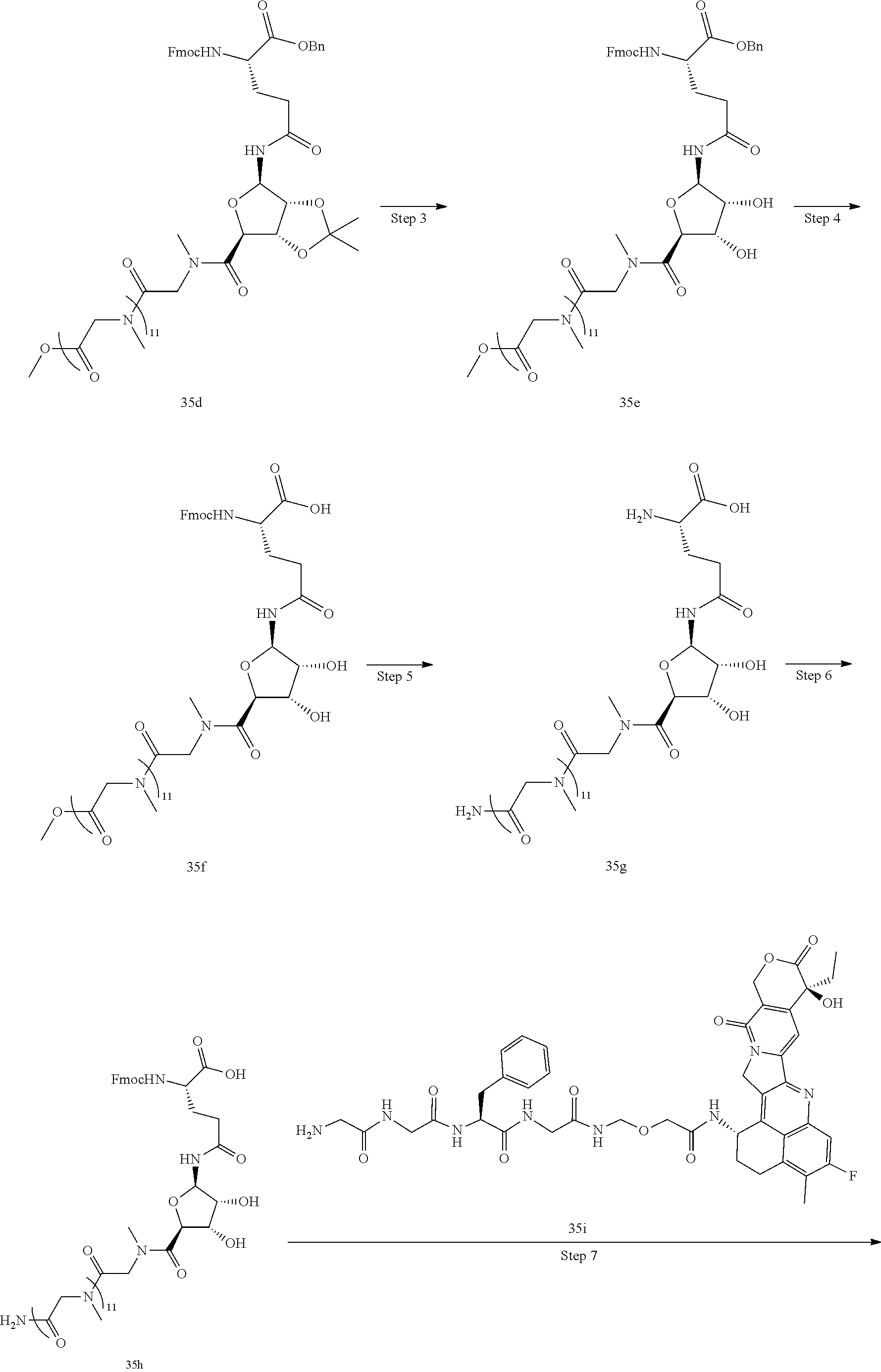

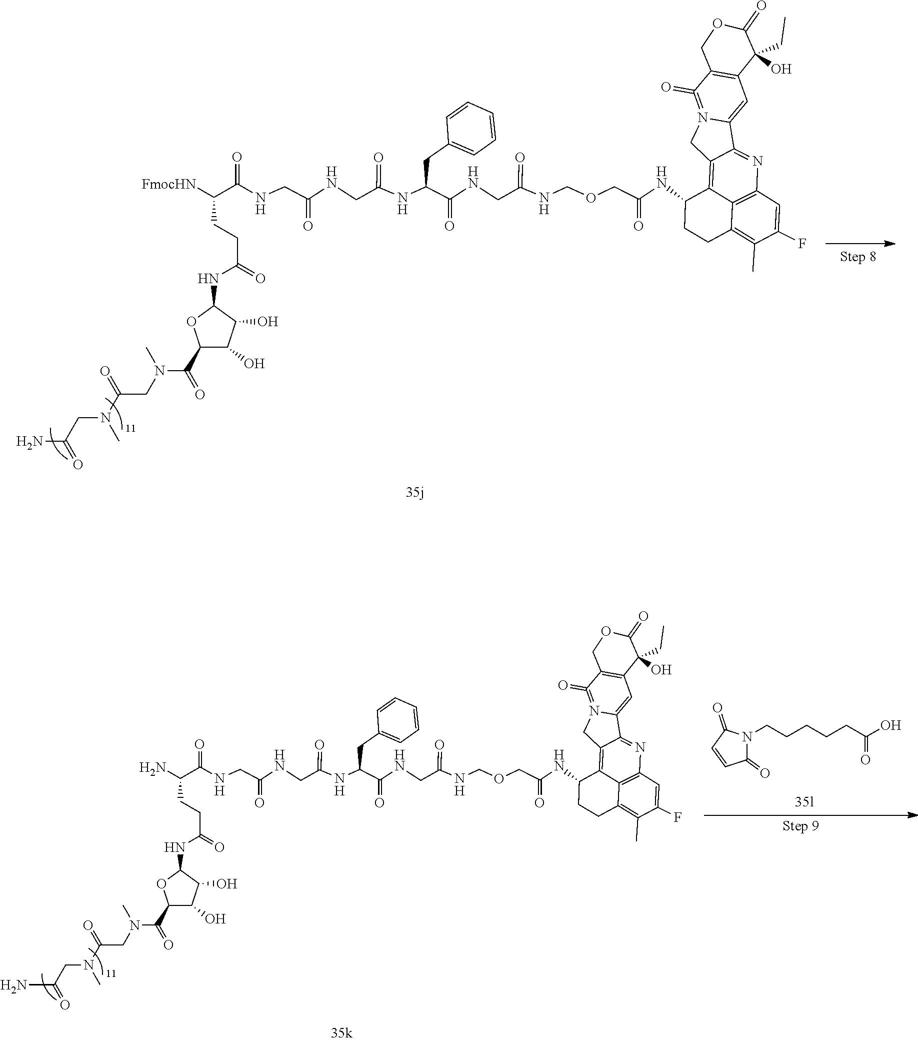

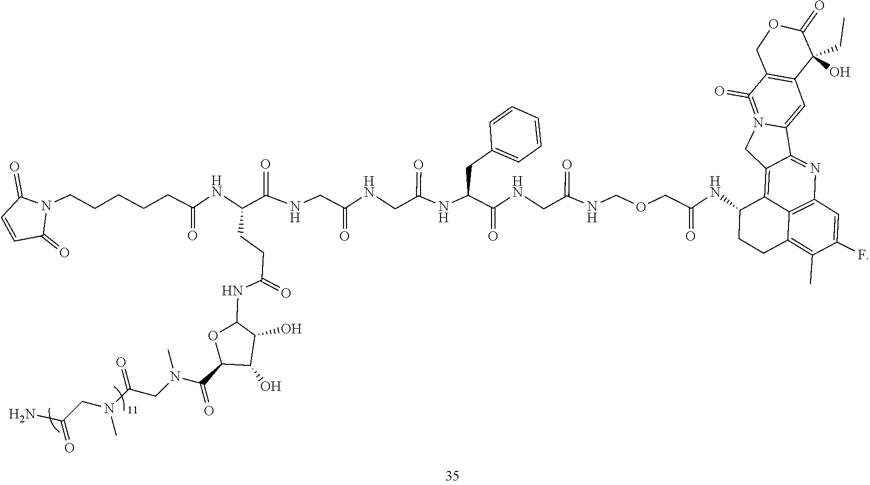

### Step 1

### N-(2-amino-2-oxoethyl)-2-(N,5,8,11,14,17,20,23,26,29-decamethyl-4,7,10,13,16,19,22,25,28-nonaoxo-2,5,8,11,14,17,20,23,26,29-decaazahentriacontan-3 I-amido)-N-methylacetamide (35b)

To a solution of compound 35a (200 mg, 0.196 mmol) in MeOH (4 mL) was added wet Pd/C (20 mg, 10 wt %). The mixture was stirred at r.t. for 2 h under H2 (15 psi) atmosphere. The mixture was filtered through a pad of celite, concentrated to give the crude product. Compound 35b (185 mg, crude) was obtained as colorless oil.

### Step 2

### Benzyl N2-(((9H-fluoren-9-yl)methoxy)carbonyl)-N5-((3 aR,6S,6aS)-6-((35-amino-3,6,9,12,15,18,21,24,27,30,33-undecamethyl-2,5,8,11,14,17,20,23,26,29,32,35-dodecaoxo-3,6,9,12,15,18,21,24,27,30,33-undecaazapentatriacontyl)(methyl)carbamoyl)-2,2-dimethyltetrahydrofuro[3,4-d][1,3]dioxol-4-yl)-L-glutaminate (35d)

To a solution of compound 35c (134 mg, 0.208 mmol) in pyridine (4 mL) were added 35b (185 mg, 0.208 mmol) and EDCI (47 mg, 0.01 mmol). The mixture was stirred at r.t. for 6 h. The mixture was concentrated and the residue was purified by prep-HPLC (Method: column: XBridge Prep C18 OBD 5 μm 19*250 mm; Mobile phase: A—water (0.1% formic acid): B—acetonitrile; Flow rate: 20 mL/min). Compound 35d (305 mg, 98.1% yield) was obtained as white solid.

MS (ESI) m/z: 1535.0 [M+Na]+.

### Step 3

### Benzyl N2-(((9H-fluoren-9-yl)methoxy)carbonyl)-N5-((3R,4S,5S)-5-((35-amino-3,6,9,12,15,18,21,24,27,30,33-undecamethyl-2,5,8,11,14,17,20,23,26,29,32,35-dodecaoxo-3,6,9,12,15,18,21,24,27,30,33-undecaazapentatriacontyl)(methyl)carbamoyl)-3,4-dihydroxytetrahydrofuran-2-yl)-L-glutaminate (35e)

To a solution of compound 35d (305 mg, 0.202 mmol) in THE (2 mL) was added THF-H2O (v:v=4:1, 10 mL). The mixture was stirred at r.t. for 4 h. The mixture was concentrated to give the crude product, which was used for next step without further purification. Compound 35e (295 mg, crude) was obtained as white solid.

MS (ESI) m/z: 1495.0 [M+Na]+.

### Step 4

### N2-(((9H-fluoren-9-yl)methoxy)carbonyl)-N5-((3R,4S,5S)-5-((35-amino-3,6,9,12,15,18,21,24,27,30,33-undecamethyl-2,5,8,11,14,17,20,23,26,29,32,35-dodecaoxo-3,6,9,12,15,18,21,24,27,30,33-undecaazapentatriacontyl)(methyl)carbamoyl)-3,4-dihydroxytetrahydrofuran-2-yl)-L-glutamine (35f)

To a solution of compound 35e (295 mg, 0.2 mmol) in MeOH (6 mL) was added wet Pd/C (30 mg, 10%). The mixture was stirred at r.t. under H2 (15 psi) for 5 h. The mixture was filtered through a pad of celite, concentrated. The crude was purified by prep-HPLC (Method: column: XBridge Prep C18 OBD Sum 19*250 mm; Mobile phase: A—water (0.1% formic acid): B—acetonitrile; Flow rate: 20 mL/min). Compound 35f (162 mg, 58.5% yield) was obtained as white solid.

MS (ESI) m/z: 1404.9 [M+Na]+.

### Step 5

### N5-((3R,4S,5S)-5-((35-amino-3,6,9,12,15,18,21,24,27,30,33-undecamethyl-2,5,8,11,14,17,20,23,26,29,32,35-dodecaoxo-3,6,9,12,15,18,21,24,27,30,33-undecaazapentatriacontyl)(methyl)carbamoyl)-3,4-dihydroxytetrahydrofuran-2-yl)-L-glutamine (35 g)

A solution of compound 35f (162 mg, 0.117 mmol) in NH3-MeOH (6 mL, 7 M) was stirred at 40° C. in a sealed tube for 16 h. The mixture was concentrated, resolved by H2O (15 mL), washed by EA (4*10 mL). The aqueous solution was lyophilized to give the crude product. Compound 35g (142 mg, crude) was obtained as white solid.

MS (ESI) m/z: 1145.9 [M+H]+.

### Step 6

### N2-(((9H-fluoren-9-yl)methoxy)carbonyl)-N5-((3R,4S,5S)-5-((35-amino-3,6,9,12,15,18,21,24,27,30,33-undecamethyl-2,5,8,11,14,17,20,23,26,29,32,35-dodecaoxo-3,6,9,12,15,18,21,24,27,30,33-undecaazapentatriacontyl)(methyl)carbamoyl)-3,4-dihydroxytetrahydrofuran-2-yl)-L-glutamine (35 h)

To a solution of compound 35g (126 mg, 0.11 mmol) in CH3CN (3 mL) and H2O (1.5 mL) were added FmocOSu (40.9 mg, 0.121 mmol) and Na2CO3 (23.3 mg, 0.22 mmol). The mixture was stirred at r.t. for 2 h. The mixture was purified by prep-HPLC (Method: column: XBridge Prep C18 OBD Sum 19*250 mm; Mobile phase: A—water (0.1% formic acid): B—acetonitrile; Flow rate: 20 mL/min). Compound 35h (62 mg, 41.1% yield) was obtained as white solid.

MS (ESI) m/z: 1388.9 [M+Na]+.

### Step 7

### (9H-fluoren-9-yl)methyl ((10S,19S)-22-(((3R,4S,5S)-5-((35-amino-3,6,9,12,15,18,21,24,27,30,33-undecamethyl-2,5,8,11,14,17,20,23,26,29,32,35-dodecaoxo-3,6,9,12,15,18,21,24,27,30,33-undecaazapentatriacontyl)(methyl)carbamoyl)-3,4-dihydroxytetrahydrofuran-2-yl)amino)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15,18,22-heptaoxo-3-oxa-5,8,11,14,17-pentaazadocosan-19-yl)carbamate (35j)

To a solution of compound 35i (20 mg, 0.024 mmol) in DMF (2 mL) were added 35 h (35.8 mg, 0.026 mmol), HATU (13.6 mg, 0.04 mmol) and DIEA (6.2 mg, 0.05 mmol). The mixture was stirred at r.t. for 30 min. The mixture was purified by prep-HPLC (Method: column: XBridge Prep C18 OBD Sum 19*250 mm; Mobile phase: A—water (0.1% formic acid): B—acetonitrile; Flow rate: 20 mL/min). Compound 35j (33 mg, 63.5%) was obtained as white solid.

MS (ESI) m/z: 2189.7 [M+H]+.

### Step 8

### (S)-2-amino-N5-((3R,4S,5S)-5-((35-amino-3,6,9,12,15,18,21,24,27,30,33-undecamethyl-2,5,8,11,14,17,20,23,26,29,32,35-dodecaoxo-3,6,9,12,15,18,21,24,27,30,33-undecaazapentatriacontyl)(methyl)carbamoyl)-3,4-dihydroxytetrahydrofuran-2-yl)-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)pentanediamide (35k)

To a solution of compound 35j (33 mg, 0.015 mmol) in DMF (2 mL) was added Et2NH (22 mg, 0.30 mmol). The mixture was stirred at r.t. for 45 min. The mixture was concentrated and co-evaporated with toluene (3*2 mL). The crude was used for next step without further purification. Compound 35k (35 mg, crude) was obtained as off-white solid.

MS (ESI) m/z: 1968.1 [M+H]+.

### Step 9

### (S)—N5-((3R,4S,5S)-5-((35-amino-3,6,9,12,15,18,21,24,27,30,33-undecamethyl-2,5,8,11,14,17,20,23,26,29,32,35-dodecaoxo-3,6,9,12,15,18,21,24,27,30,33-undecaazapentatriacontyl)(methyl)carbamoyl)-3,4-dihydroxytetrahydrofuran-2-yl)-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)pentanediamide (35)

To a solution of compound 35i (4.8 mg, 0.023 mmol) in DMF (2 mL) were added HATU (8.7 mg, 0.023 mmol) and DIEA (2.96 mg, 0.023 mmol). The mixture was stirred at r.t. for 15 min. Compound 35k (30 mg, 0.015 mmol) was added into the mixture. The mixture was stirred at r.t. for 15 min. The mixture was purified by prep-HPLC (Method: column: XBridge Prep C18 OBD Sum 19*250 mm; Mobile phase: A—water (0.1% formic acid): B—acetonitrile; Flow rate: 20 mL/min). Compound 35 (19.8 mg, 60.09% yield) was obtained as white solid.

MS (ESI) m/z: 2161.4 [M+H]+.

### Example 36

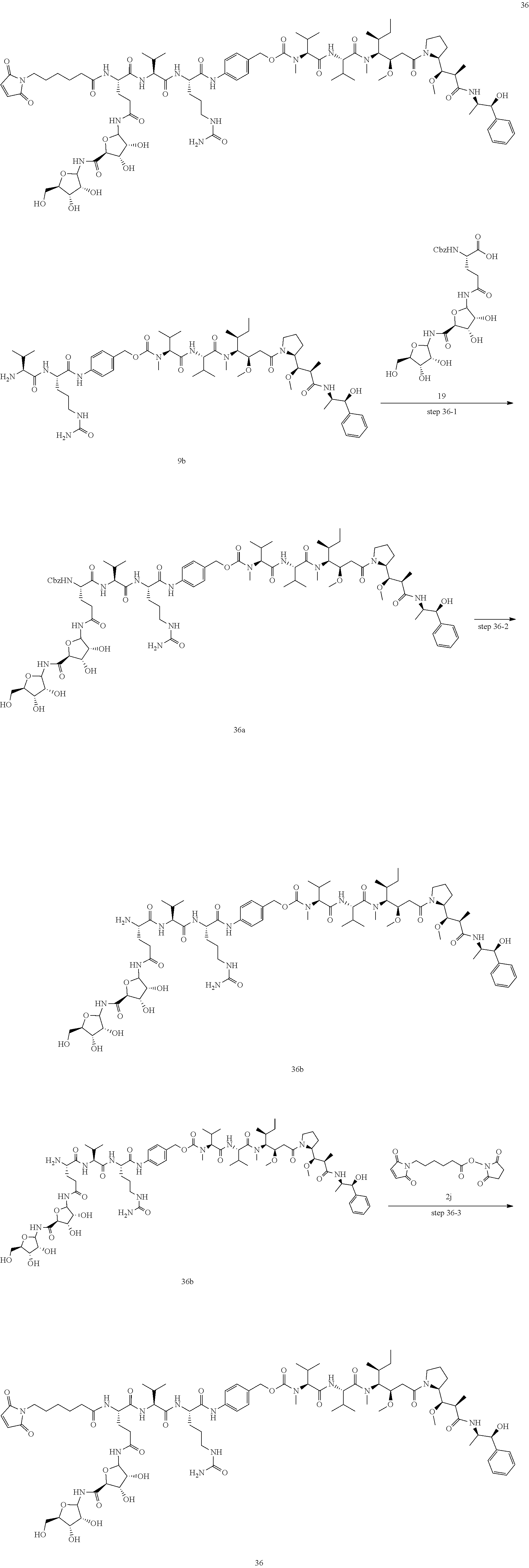

### Step 1

### 4-((5S,8S,11S)-5-(3-(((3R,4S,5S)-5-(((3R,4S,5R)-3,4-dihydroxy-5-(hydroxymethyl) tetrahydrofuran-2-yl)carbamoyl)-3,4-dihydroxytetrahydrofuran-2-yl)amino)-3-oxopropyl)-8-isopropyl-3,6,9-trioxo-1-phenyl-11-(3-ureidopropyl)-2-oxa-4,7,10-triazadodecan-12-amido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (36a)

Compound 36a (34.7 mg, 36.5% yield) was synthesized according to synthetic procedure of step 1 of example 33.

MS (ESI) m/z: 1664.4 [M+H]+

### Step 2

### 4-((2S)-2-((2S)-2-((2S)-2-amino-5-(((3R,4S,5S)-5-(((3R,4S,5R)-3,4-dihydroxy-5-(hydroxymethyl)tetrahydrofuran-2-yl)carbamoyl)-3,4-dihydroxytetrahydrofuran-2-yl)amino)-5-oxopentanamido)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (36b)

Compound 36b (19.2 mg, 60.2% yield) was synthesized according to synthetic procedure of step 2 f example 33.

MS (ESI) m/z: 1529.2 [M+H]+

### Step 3

### 4-((2S)-2-((2S)-2-((2S)-5-(((3R,4S,5S)-5-(((3R,4S,5R)-3,4-dihydroxy-5-(hydroxymethyl)tetrahydrofuran-2-yl)carbamoyl)-3,4-dihydroxytetrahydrofuran-2-yl)amino)-2-(6-(2,5-dioxo-2,5-dihydro-1H-pyrrol-1-yl)hexanamido)-5-oxopentanamido)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (36)

Compound 36 (10.7 mg, 49.5% yield) was synthesized according to synthetic procedure of step 8 of example 2.

MS (ESI) m/z: 1722.9 [M+H]+

### Example 37

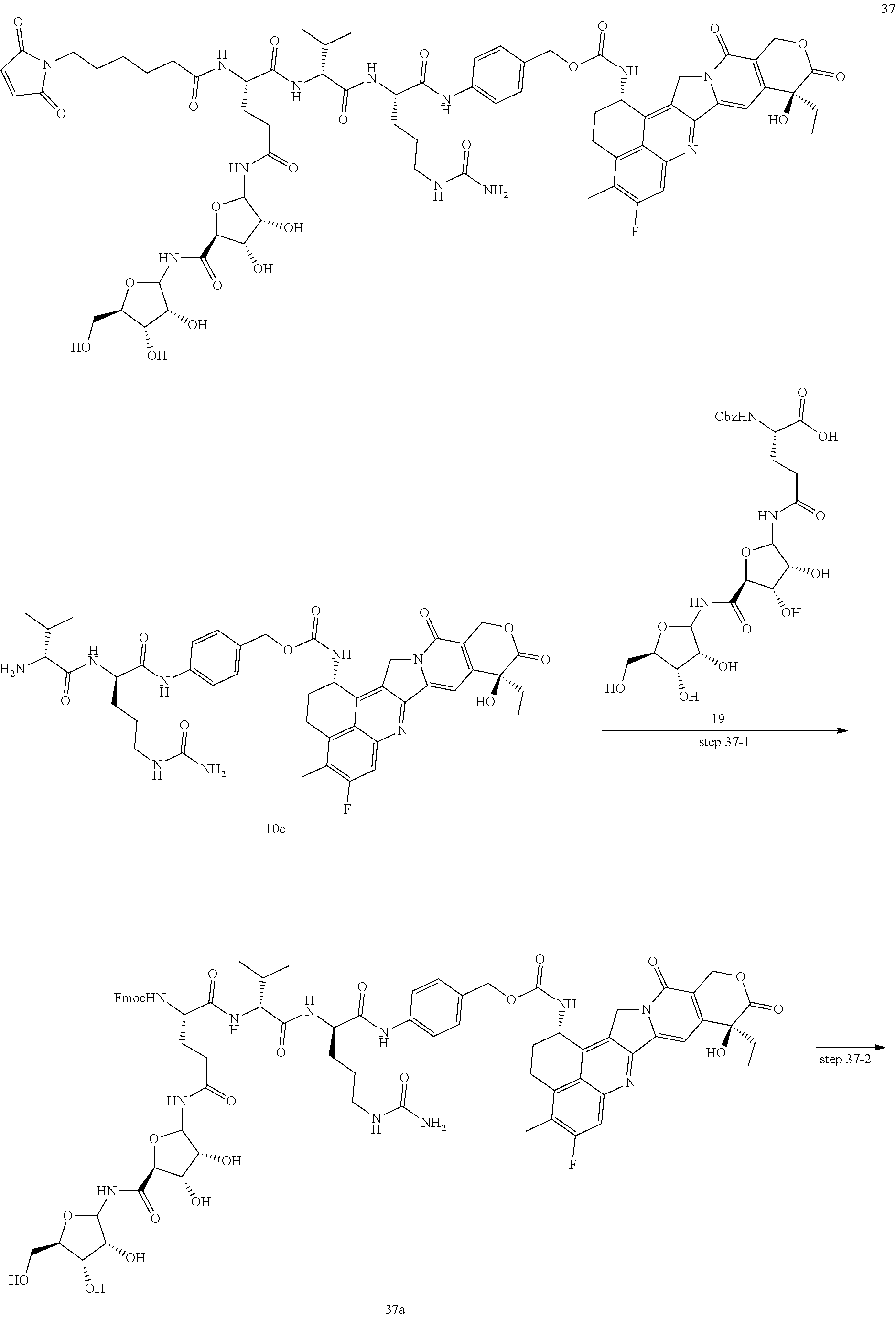

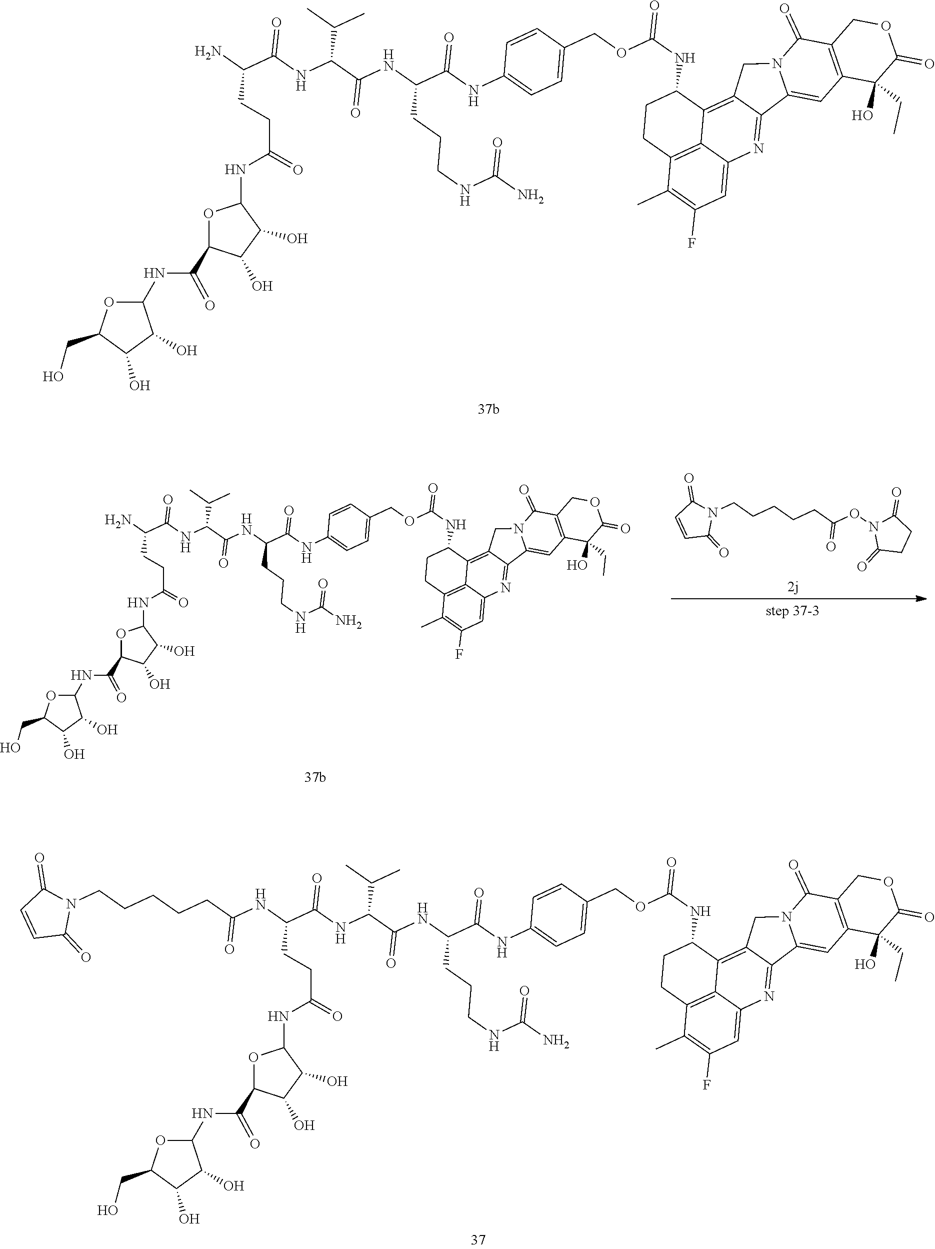

### Step 1

### 4-((5S,8S,11S)-5-(3-(((3R,4S,5S)-5-(((3R,4S,5R)-3,4-dihydroxy-5-(hydroxymethyl) tetrahydrofuran-2-yl)carbamoyl)-3,4-dihydroxytetrahydrofuran-2-yl)amino)-3-oxopropyl)-8-isopropyl-3,6,9-trioxo-1-phenyl-11-(3-ureidopropyl)-2-oxa-4,7,10-triazadodecan-12-amido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (37a)

Compound 37a (15.0 mg, 24.0% yield) was synthesized according to synthetic procedure of step 1 of example 33.

MS (ESI) m/z: 1381.8 [M+H]+

### Step 2

### 4-((2S)-2-((2S)-2-((2S)-2-amino-5-(((3R,4S,5S)-5-(((3R,4S,5R)-3,4-dihydroxy-5-(hydroxymethyl)tetrahydrofuran-2-yl)carbamoyl)-3,4-dihydroxytetrahydrofuran-2-yl)amino)-5-oxopentanamido)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (37b)

Compound 37b (12.5 mg, 90.9% yield) was synthesized according to synthetic procedure of step 2 f example 33.

MS (ESI) m/z: 1268.9 [M+Na]+

### Step 3

### 4-((2S)-2-((2S)-2-((2S)-5-(((3R,4S,5S)-5-(((3R,4S,5R)-3,4-dihydroxy-5-(hydroxymethyl)tetrahydrofuran-2-yl)carbamoyl)-3,4-dihydroxytetrahydrofuran-2-yl)amino)-2-(6-(2,5-dioxo-2,5-dihydro-1H-pyrrol-1-yl)hexanamido)-5-oxopentanamido)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (37)

Compound 37 (7.3 mg, 50.6% yield) was synthesized according to synthetic procedure of step 8 of example 2.

MS (ESI) m/z: 1441.0 [M+H]+

### Example 38

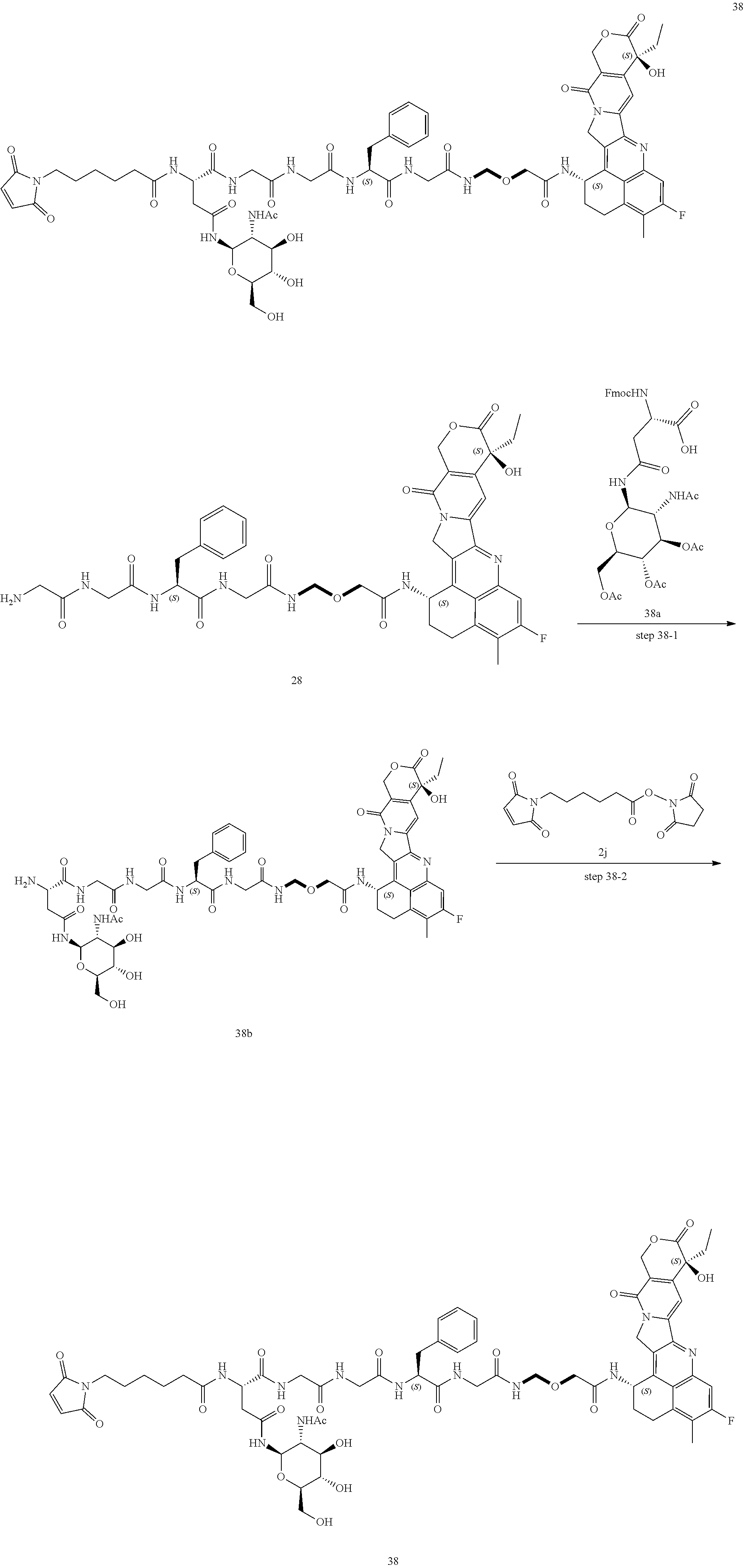

### Step 1

### (S)—N4-((2R,3R,4R,5S,6R)-3-acetamido-4,5-dihydroxy-6-(hydroxymethyl)tetrahydro-2H-pyran-2-yl)-2-amino-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)succinimide (38b)

To a mixture of 38a (30 mg, 0.044 mmol) and TSTU (13.21 mg, 0.044 mmol) in DMF (1 mL) was added DIPEA (17.01 mg, 0.132 mmol). The mixture was reacted at r.t. for 10 min and the acid was converted to active ester based on LCMS. 28 (29.52 mg, 0.035 mmol) was added and reacted at the same temperature for another 1 h. On completion of the reaction, NH2NH2·H2O (200 uL, 80% was added and the mixture was left at r.t. for another 10 min. The mixture was filtered and the filtrate was purified using prep-HPLC (Method: column: XBridge Prep C18 OBD Sum 19*250 mm; Mobile phase: A—water (0.1% formic acid): B—acetonitrile; Flow rate: 20 mL/min) to provide 38b (6.9 mg, 17.2% yield).

MS (ESI) m/z: 1158.9 [M+H]+

### Step 2

### (S)—N4-((2R,3R,4R,5S,6R)-3-acetamido-4,5-dihydroxy-6-(hydroxymethyl)tetrahydro-2H-pyran-2-yl)-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)succinimide (38)

Compound 38 (4.5 mg, 55.9% yield) was synthesized according to synthetic procedure of step 8 of example 2.

MS (ESI) m/z: 1373.8 [M+Na]+

### Example 39

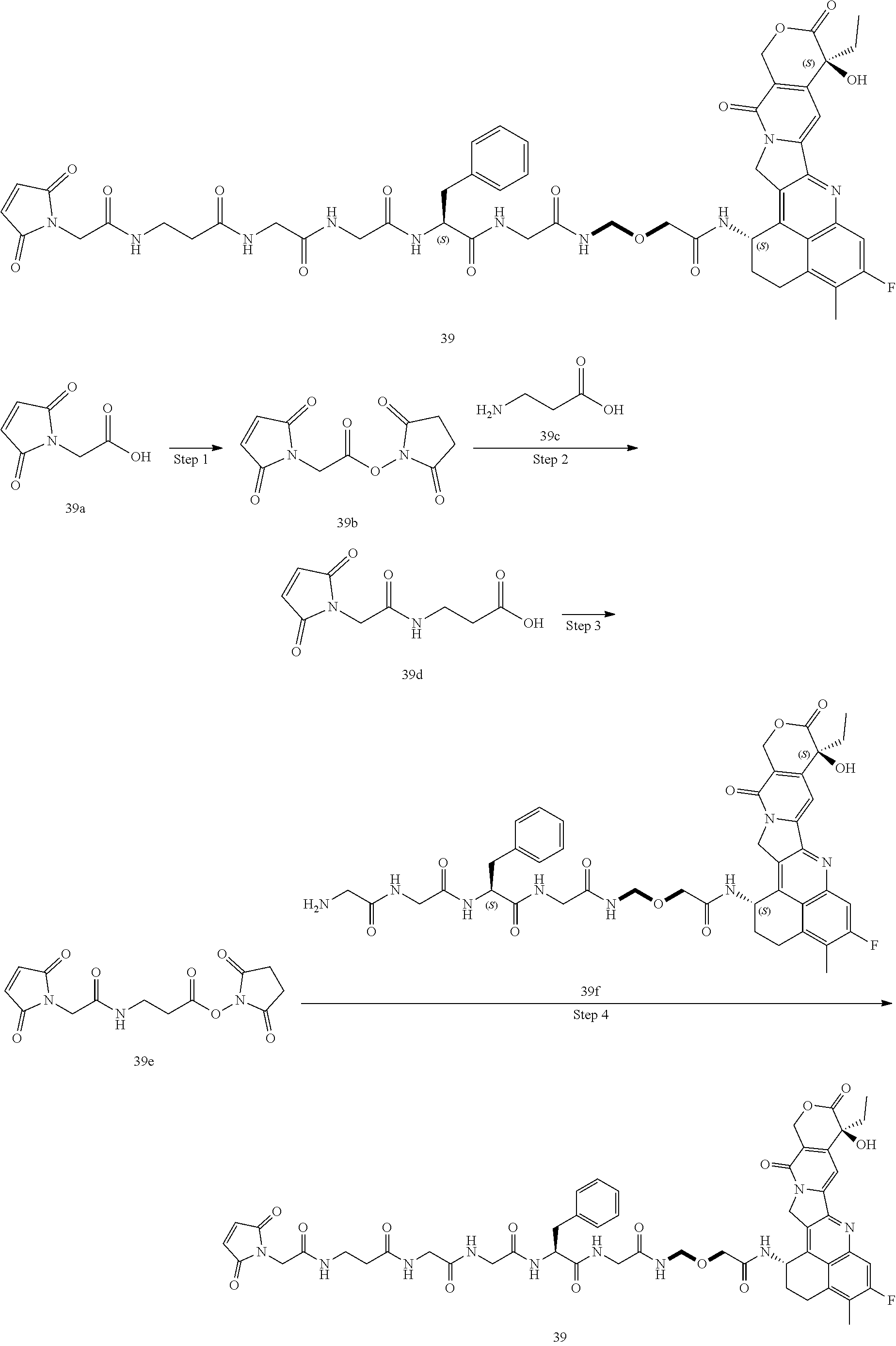

### Step 1

### 2,5-dioxopyrrolidin-1-yl 2-(2,5-dioxo-2,5-dihydro-1H-pyrrol-1-yl)acetate (39b)

To a solution of compound 39a (600 mg, 3.87 mmol) in DMF (8 mL) were added DCC (958 mg, 4.64 mmol) and HOSu (534 mg, 4.64 mmol). The mixture was stirred at r.t. for 4 h. The mixture was filtered to remove the solid, the filtrate was concentrated to give the crude product. Compound 39b (855 mg, crude) was obtained as off-white solid.

MS (ESI) m/z: 275.2 [M+Na]+.

### Step 2

### 3-(2-(2,5-dioxo-2,5-dihydro-1H-pyrrol-1-yl)acetamido)propanoic acid (39d)

To a solution of compound 39b (855 mg, crude) in DMSO (8 mL) was added 3-aminopropanoic acid (379 mg, 4.26 mmol). The mixture was stirred at 40° C. for 4 h. The mixture was diluted with H2O (20 mL), washed by MTBE (3*20 mL). The aqueous solution was extracted by EA (6*100 mL). The organic layer was combined, dried over anhydrous Na2SO4, filtered and concentrated. A solution of compound 39d (875 mg, theoretical amount) in DMSO was obtained, used for directly in the next step without further purification.

MS (ESI) m/z: 227.2 [M+H]+.

### Step 3

### 2,5-dioxopyrrolidin-1-yl 3-(2-(2,5-dioxo-2,5-dihydro-1H-pyrrol-1-yl)acetamido)propanoate

To a solution of compound (875 mg, theoretical yield) in DCM (10 mL) were added DCC (958 mg, 4.64 mmol) and HOSu (534 mg, 4.64 mmol). The mixture was stirred at r.t. for 3 h. The mixture was filtered and concentrated. The mixture was purified by prep-HPLC (Method: column: XBridge Prep C18 OBD 5 μm 19*250 mm; Mobile phase: A—water (0.1% formic acid): B—acetonitrile; Flow rate: 20 mL/min). Compound 39e (195 mg, 15.6% yield) was obtained as colorless gum.

MS (ESI) m/z: 346.3 [M+Na]+.

### Step 4

### (S)-2-(2-(2-(3-(2-(2,5-dioxo-2,5-dihydro-1H-pyrrol-1-yl)acetamido)propanamido)acetamido)acetamido)-N-(2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)-3-phenylpropanamide (39)

To a solution of compound 39f (25 mg, 0.03 mmol) in DMF (0.8 mL) were added 39e (12.5 mg, 0.04 mmol) and DIEA (2.2 mg, 0.03 mmol). The mixture was stirred at r.t. for 2 h. The pH was adjusted to 6 by 0.1% TFA solution. The mixture was purified by prep-HPLC (Method: column: XBridge Prep C18 OBD Sum 19*250 mm; Mobile phase: A—water (0.1% formic acid): B—acetonitrile; Flow rate: 20 mL/min). Compound 39 (8.0 mg, 25.6% yield) was obtained as white solid.

MS (ESI) m/z: 1071.7 [M+Na]+.

### Example 40

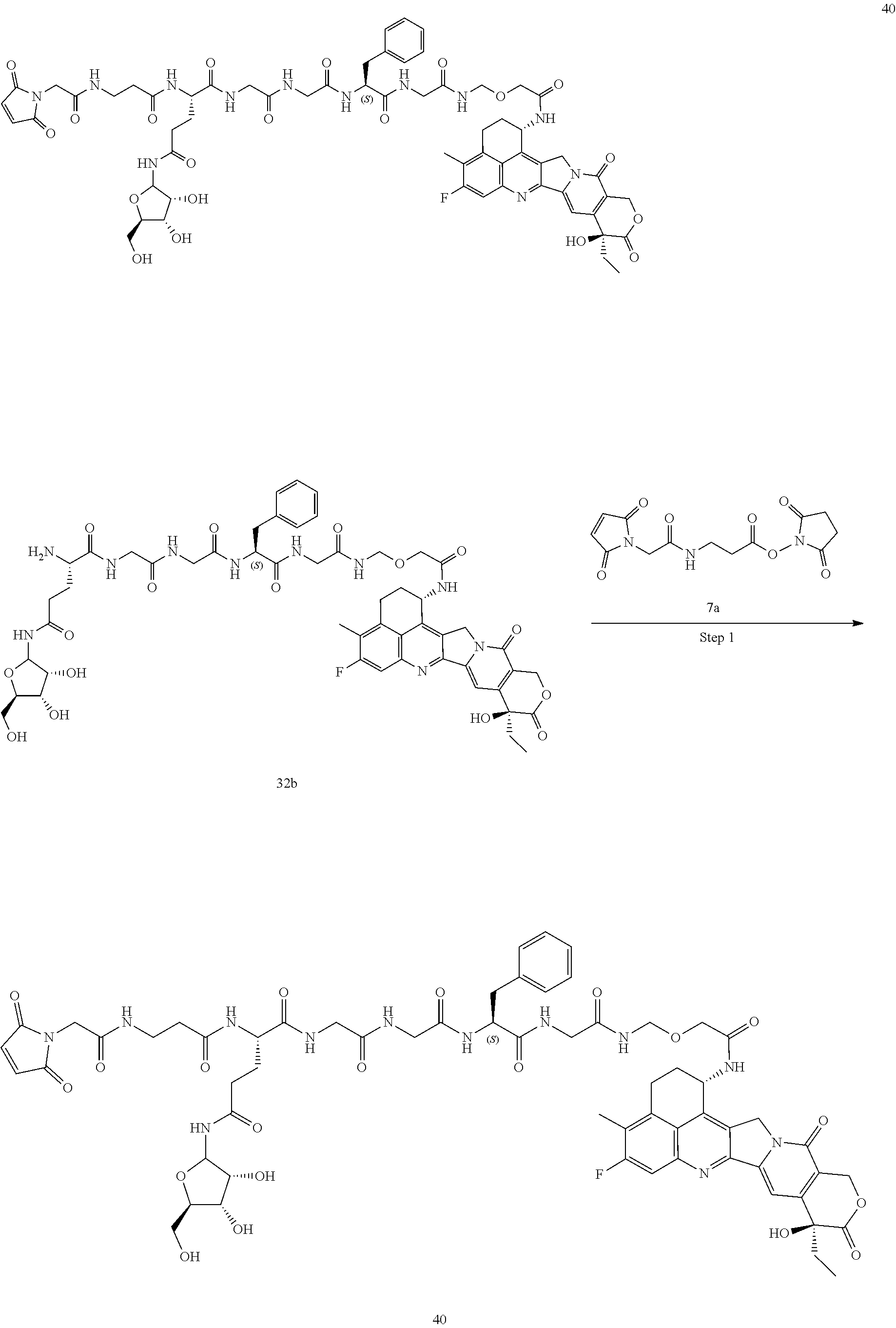

### Step 1

### (2S)—N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-((3R,4S,5R)-3,4-dihydroxy-5-(hydroxymethyl)tetrahydrofuran-2-yl)-2-(3-(2-(2,5-dioxo-2,5-dihydro-1H-pyrrol-1-yl)acetamido)propanamido)pentanediamide (40)

To the solution of 32b (20 mg, 18.2 mol) in dry DMF (1 mL) was added 7a (8.8 mg, 27.2 mol), stirred at r.t. for 1.5 h. The solution was filtered and purified by prep-HPLC (0.1% FA in water/MeCN), lyophilized to give the title compound 40 (9.5 mg, 39.9% yield) as a white solid.

MS (ESI) m/z: 1331.8 [M+Na]+

### Example 41

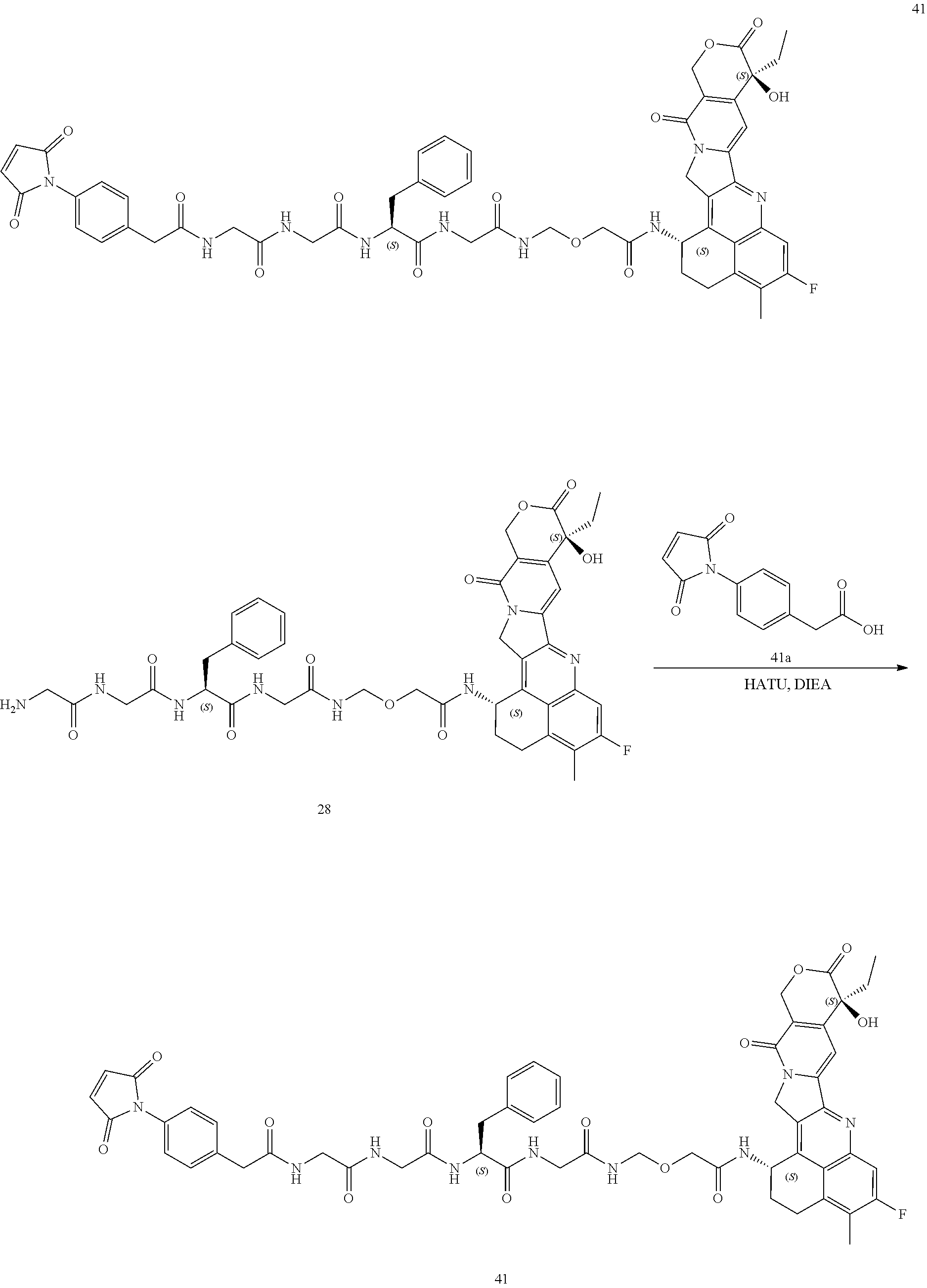

(S)-2-(2-(2-(2-(4-(2,5-dioxo-2,5-dihydro-1H-pyrrol-1-yl)phenyl)acetamido)acetamido)acetamido)-N-(2-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-2-oxoethyl)-3-phenylpropanamide (41)

28 (synthesized according to synthetic procedure of example 28, 5.0 mg, 0.022 mmol), 41a (commercially available, 18 mg, 0.022 mmol) and HATU (9.0 mg, 0.024 mmol) were dissolved in DMF (0.5 mL) followed by the addition of DIEA (11.5 μL, 0.065 mmol). The resulting mixture was stirred at 25° C. for 15 min. After complete reaction, the reaction mixture was acidified to pH=6 with 0.1% TFA in H2O and purified by prep-HPLC (Method: column: XBridge Prep C18 OBD Sum 19*250 mm; Mobile phase: A—water (0.1% formic acid): B—acetonitrile; Flow rate: 20 mL/min) to afford 41 (11 mg, 48.2% yield) as a white solid.

MS (ESI) m/z: 1076.5 [M+Na]+

### Example 42

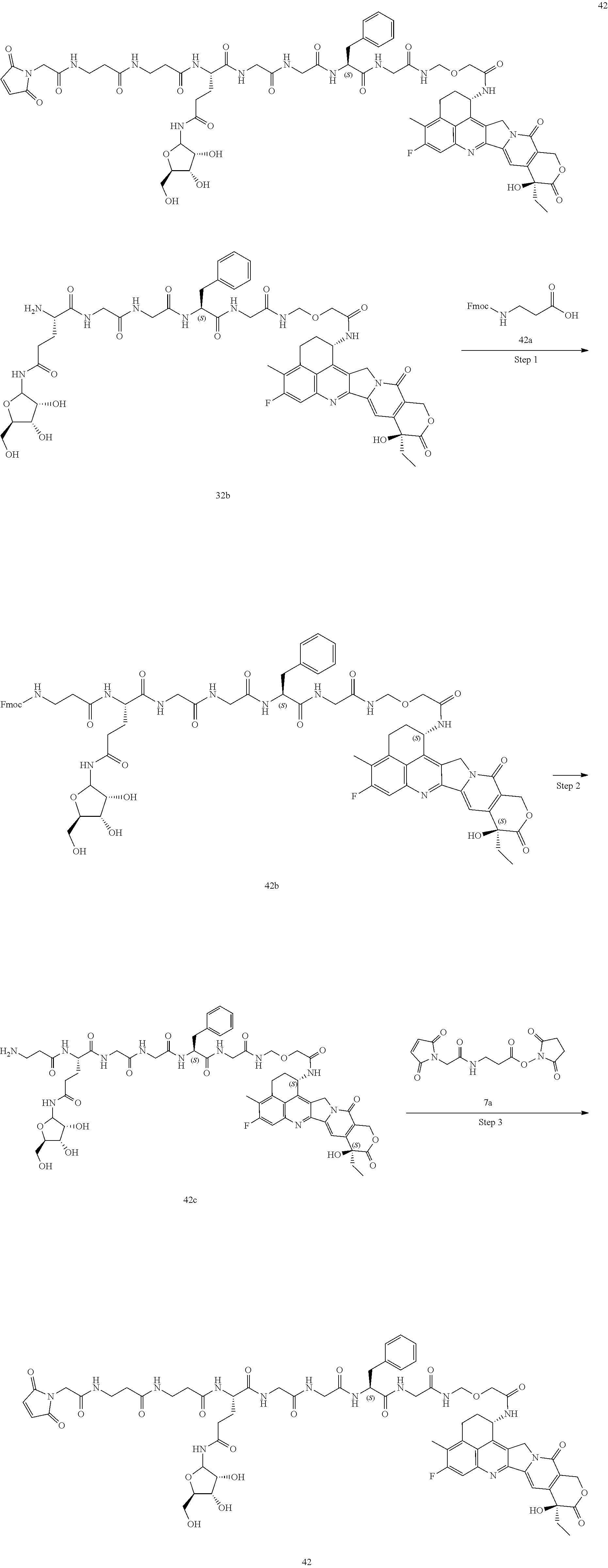

### Step 1

### (9H-fluoren-9-yl)methyl ((10S,19S)-10-benzyl-19-(3-(((3R,4S,5R)-3,4-dihydroxy-5-(hydroxymethyl)tetrahydrofuran-2-yl)amino)-3-oxopropyl)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15,18,21-heptaoxo-3-oxa-5,8,11,14,17,20-hexaazatricosan-23-yl)carbamate (42b)

To the solution of 32b (24 mg, 21.8 mol), Fmoc-glycine 42a (7.5 mg, 24.0 mol) and HATU (9.1 mg, 24.0 mol) in dry DMF (1.5 mL) was added DIEA (7 μL, 43.6 mol), stirred at r.t. for 25 min. The solution was filtered and purified by prep-HPLC (0.1% FA in water/MeCN), lyophilized to give the title compound 42b (16.5 mg, 54.3% yield) as a white solid.

MS (ESI) m/z: 1416.8 [M+Na]+

### Step 2

### (2S)-2-(3-aminopropanamido)-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-((3R,4S,5R)-3,4-dihydroxy-5-(hydroxymethyl)tetrahydrofuran-2-yl)pentanediamide (42c)

To the solution of 42b (16.5 mg, 11.8 mol) in dry DMF (1 mL) was added Et2NH (24 μL, 0.24 mmol), stirred at r.t. for 30 min. The solution was concentrated under vacuum. The residue was triturated with MTBE (5 mL), filtered and washed with MTBE (1 mL*3). The filter cake was collected to give the title compound 42c (15 mg, 108.4% yield) as a light brown solid, which was used directly for the next step without further purification.

MS (ESI) m/z: 1172.8 [M+H]+

### Step 3

### (2S)—N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-((3R,4S,5R)-3,4-dihydroxy-5-(hydroxymethyl)tetrahydrofuran-2-yl)-2-(3-(3-(2-(2,5-dioxo-2,5-dihydro-1H-pyrrol-1-yl)acetamido)propanamido)propanamido)pentanediamide (42)

The title compound 42 (9.1 mg, 55.2% yield) was obtained according to the procedure described in Step 1 of Example 40.

MS (ESI) m/z: 1402.8 [M+Na]+

### Example 43

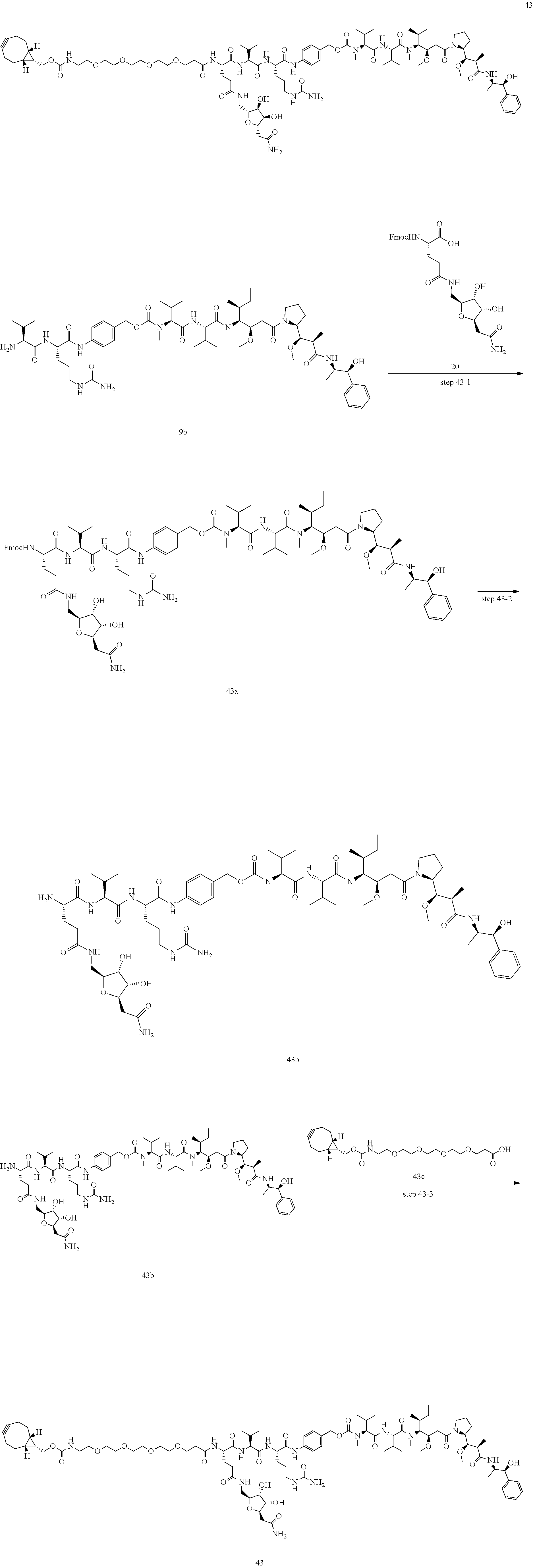

### Step 1

### 4-((5S,8S,11S)-5-(3-((((2S,3R,4S,5R)-5-(2-amino-2-oxoethyl)-3,4-dihydroxytetrahydrofuran-2-yl)methyl)amino)-3-oxopropyl)-1-(9H-fluoren-9-yl)-8-isopropyl-3,6,9-trioxo-11-(3-ureidopropyl)-2-oxa-4,7,10-triazadodecan-12-amido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (43a)

To a mixture of 20 (25 mg, 0.046 mmol) and HATU (17.55 mg, 0.046 mmol) in DMF (1 mL) was added DIPEA (17.90 mg, 0.138 mmol). The mixture was reacted at r.t. for 10 min. 9b (51.86 mg, 0.046 mmol) was added and reacted at the same temperature for another 15 min. On completion of the reaction, The mixture was filtered and the filtrate was purified using prep-HPLC (Method: column: XBridge Prep C18 OBD Sum 19*250 mm; Mobile phase: A—water (0.1% formic acid): B—acetonitrile; Flow rate: 20 mL/min) to provide 43a (55.9 mg, 73.5% yield).

MS (ESI) m/z: 824.6 [M+2H]2+

### Step 2

### 4-((S)-2-((S)-2-((S)-2-amino-5-((((2S,3R,4S,5R)-5-(2-amino-2-oxoethyl)-3,4-dihydroxytetrahydrofuran-2-yl)methyl)amino)-5-oxopentanamido)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (43b)

To a mixture of 43a (55.9 mg, 0.034 mmol) in DMF (1 mL) was added Et2N (49.65 mg, 0.479 mmol). The mixture was reacted at r.t. for 30 min. On completion of the reaction, The mixture was concentrated under reduce pressure to provide 43b (59 mg, crude).

MS (ESI) m/z: 713.5 [M+2H]2+

### Step 3

### 4-((21S,24S,27S)-21-(3-((((2S,3R,4S,5R)-5-(2-amino-2-oxoethyl)-3,4-dihydroxytetrahydrofuran-2-yl)methyl)amino)-3-oxopropyl)-1-((1R,8S,9s)-bicyclo[6.1.0]non-4-yn-9-yl)-24-isopropyl-3,19,22,25-tetraoxo-27-(3-ureidopropyl)-2,7,10,13,16-pentaoxa-4,20,23,26-tetraazaoctacosan-28-amido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (43)

To a mixture of 43c (10.0 mg, 0.023 mmol) and HATU (8.61 mg, 0.023 mmol) in DMF (0.3 mL) was added DIPEA (8.78 mg, 0.068 mmol). The mixture was reacted at r.t. for 10 min. 43b (32.27 mg, crude) was added and reacted at the same temperature for another 15 min. On completion of the reaction, The mixture was filtered and the filtrate was purified using prep-HPLC (Method: column: XBridge Prep C18 OBD Sum 19*250 mm; Mobile phase: A—water (0.1% formic acid): B—acetonitrile; Flow rate: 20 mL/min) to provide 43 (22.2 mg, 53.0% yield).

MS (ESI) m/z: 1849.2 [M+H]+

### Example 44

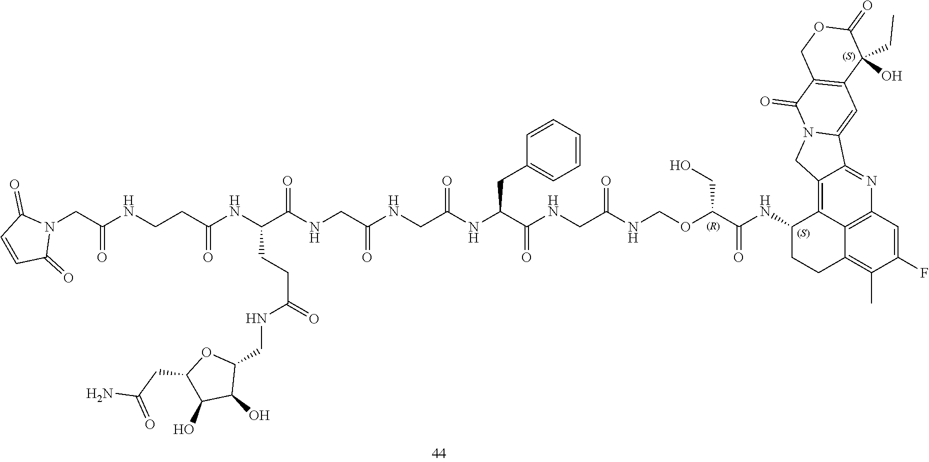

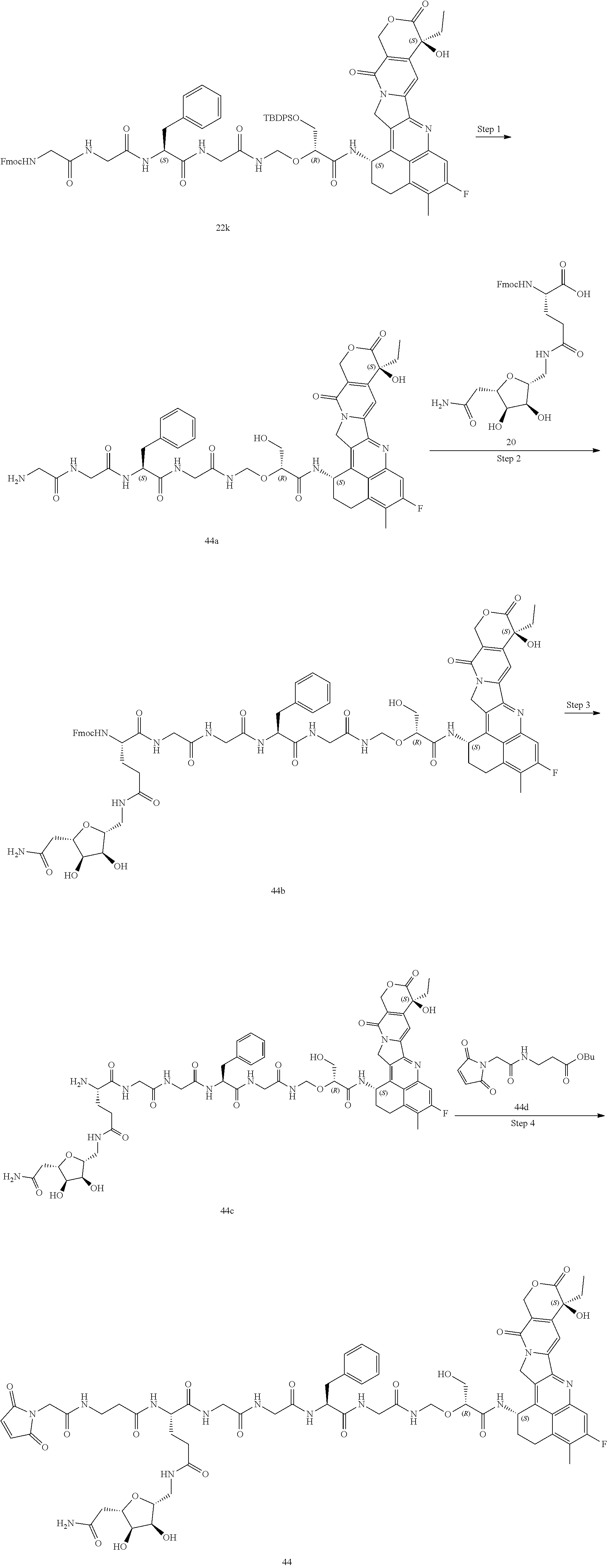

### Step 1

### (9H-fluoren-9-yl)methyl ((2R,10S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-(hydroxymethyl)-1,6,9,12,15-pentaoxo-3-oxa-5,8,11,14-tetraazahexadecan-16-yl)carbamate (44a)

22k (125 mg, 0.094 mmol) was dissolved in THE (3 mL) followed by the addition of TBAF (1 M stock solution in THF, 113 μL, 0.11 mmol). The resulting mixture was stirred at 25° C. for 1 h. Et2NH (97 μL, 0.94 mmol) was added and the mixture was further stirred at 25° C. for 1 h. After complete reaction, the reaction mixture was purified by prep-HPLC (Method: column: XBridge Prep C18 OBD Sum 19*250 mm; Mobile phase: A—water (0.1% formic acid): B—acetonitrile; Flow rate: 20 mL/min) to afford 44a (38 mg, 46.4% yield) as a white solid.

MS (ESI) m/z: 871.7 [M+H]+

### Step 2

### (9H-fluoren-9-yl)methyl ((6S,15S,23R)-1-((2R,3S,4R,5S)-5-(2-amino-2-oxoethyl)-3,4-dihydroxytetrahydrofuran-2-yl)-15-benzyl-24-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-23-(hydroxymethyl)-3,7,10,13,16,19,24-heptaoxo-22-oxa-2,8,11,14,17,20-hexaazatetracosan-6-yl)carbamate (44b)

44b (14.5 mg, 50.3% yield) was synthesized according to the synthetic procedure of step 8 of example 22.

MS (ESI) m/z: 1416.9 [M+Na]+

### Step 3

### (S)-2-amino-N5-(((2R,3S,4R,5S)-5-(2-amino-2-oxoethyl)-3,4-dihydroxytetrahydrofuran-2-yl)methyl)-N1-((2R,10 S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-(hydroxymethyl)-1,6,9,12,15-pentaoxo-3-oxa-5,8,11,14-tetraazahexadecan-16-yl)pentanediamide (44c)

44c (15.2 mg, crude) was synthesized according to the synthetic procedure of step 9 of example 22.

MS (ESI) m/z: 1173.8 [M+H]+

### Step 4

### (S)—N5-(((2R,3S,4R,5S)-5-(2-amino-2-oxoethyl)-3,4-dihydroxytetrahydrofuran-2-yl)methyl)-N1-((2R,10S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-(hydroxymethyl)-1,6,9,12,15-pentaoxo-3-oxa-5,8,11,14-tetraazahexadecan-16-yl)-2-(3-(2-(2,5-dioxo-2,5-dihydro-1H-pyrrol-1-yl)acetamido)propanamido)pentanediamide (44c)

44 (5.3 mg, 36.9% yield) was synthesized according to the synthetic procedure of step 10 of example 22.

MS (ESI) m/z: 1402.9 [M+Na]+

### Example 45

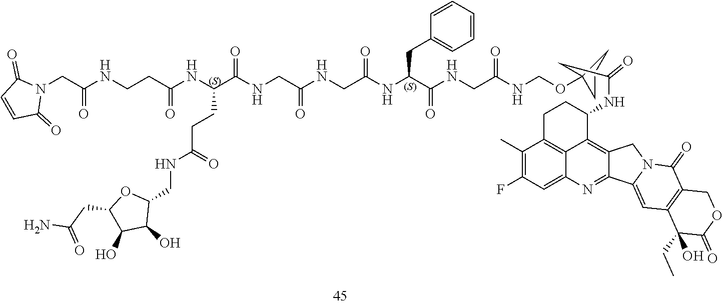

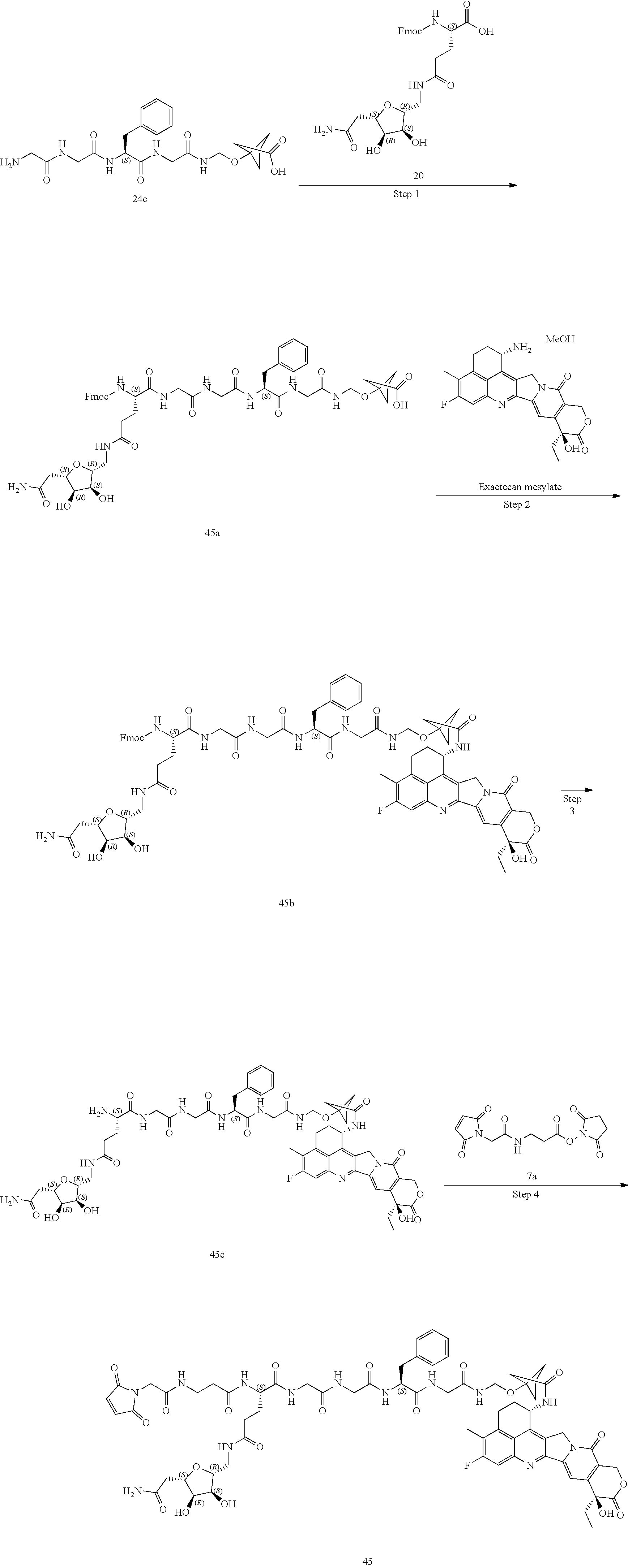

### Step 1

### 3-(((5S,14S)-5-(3-((((2R,3S,4R,5S)-5-(2-amino-2-oxoethyl)-3,4-dihydroxytetrahydrofuran-2-yl)methyl)amino)-3-oxopropyl)-14-benzyl-1-(9H-fluoren-9-yl)-3,6,9,12,15,18-hexaoxo-2-oxa-4,7,10,13,16,19-hexaazaicosan-20-yl)oxy)bicyclo[1.1.1]pentane-1-carboxylic acid (45a)

To the solution of 20 (68 mg, 0.12 mmol) and TSTU (37 mg, 0.12 mmol) in dry DMF (1 mL) was added DIEA (56 μL, 0.34 mmol), stirred at r.t. for 10 min. 24c (53 mg, 0.11 mmol) was added, stirred at r.t. for 1 h. The solution was filtered and purified by prep-HPLC (0.1% FA in water/MeCN), lyophilized to give the title compound 45a (52 mg, 46.3% yield) as a white solid.

MS (ESI) m/z: 997.7 [M−H]−

### Step 2

### (9H-fluoren-9-yl)methyl ((7S,16S)-21-((2R,3S,4R,5S)-5-(2-amino-2-oxoethyl)-3,4-dihydroxytetrahydrofuran-2-yl)-7-benzyl-1-((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)bicyclo[1.1.1]pentan-1-yl)oxy)-3,6,9,12,15,19-hexaoxo-2,5,8,11,14,20-hexaazahenicosan-16-yl)carbamate (45b)

To the solution of 45a (52 mg, 52.0 mol), exatecan mesylate (29 mg, 54.7 mol) and HATU (21 mg, 54.7 mol) in dry DMF (2 mL) was added DIEA (17 μL, 0.1 mmol), stirred at r.t. for 30 min. The solution was filtered and purified by prep-HPLC (0.1% FA in water/MeCN), lyophilized to give the title compound 45b (62 mg, 84.2% yield) as an off-white solid.

MS (ESI) m/z: 1438.9 [M+Na]+

### Step 3

### (S)-2-amino-N5-(((2R,3S,4R,5S)-5-(2-amino-2-oxoethyl)-3,4-dihydroxytetrahydrofuran-2-yl)methyl)-N1-((S)-7-benzyl-1-((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)bicyclo[1.1.1]pentan-1-yl)oxy)-3,6,9,12-tetraoxo-2,5,8,11-tetraazatridecan-13-yl)pentanediamide (45c)

To the solution of 45b (60 mg, 42.4 mol) in dry DMF (1 mL) was added Et2NH (88 μL, 0.85 mmol), stirred at r.t. for 2 h. The solution was concentrated under vacuum. The residue was triturated with MTBE (5 mL), filtered and washed with MTBE (1 mL*3). The filter cake was collected to give the title compound 45c (56 mg, 110.6% yield) as a light brown solid, which was used directly for the next step without further purification.

MS (ESI) m/z: 1194.9 [M+H]+

### Step 4

### (S)—N5-(((2R,3S,4R,5S)-5-(2-amino-2-oxoethyl)-3,4-dihydroxytetrahydrofuran-2-yl)methyl)-N1-((S)-7-benzyl-1-((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)bicyclo[1.1.1]pentan-1-yl)oxy)-3,6,9,12-tetraoxo-2,5,8,11-tetraazatridecan-13-yl)-2-(3-(2-(2,5-dioxo-2,5-dihydro-1H-pyrrol-1-yl)acetamido)propanamido)pentanediamide (45)

The title compound 45 (13 mg, 44.1% yield) was obtained according to the procedure described in Step 1 of Example 40.

MS (ESI) m/z: 1424.8 [M+Na]+

### Example 46

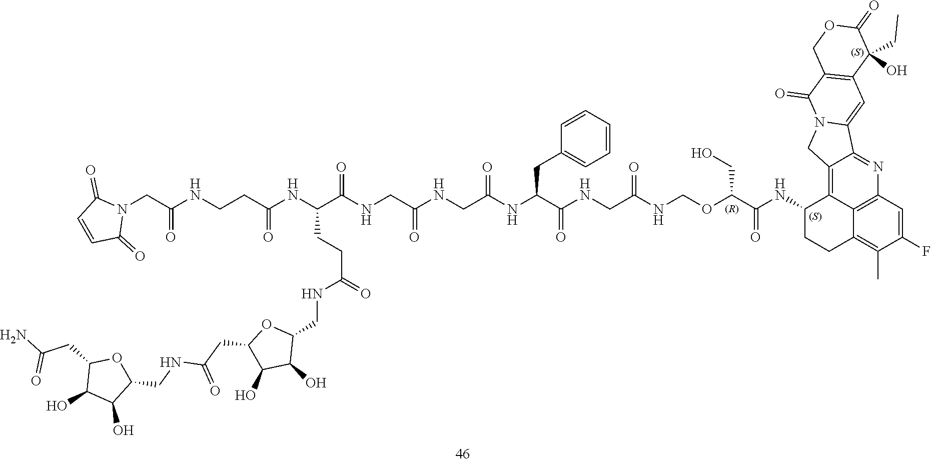

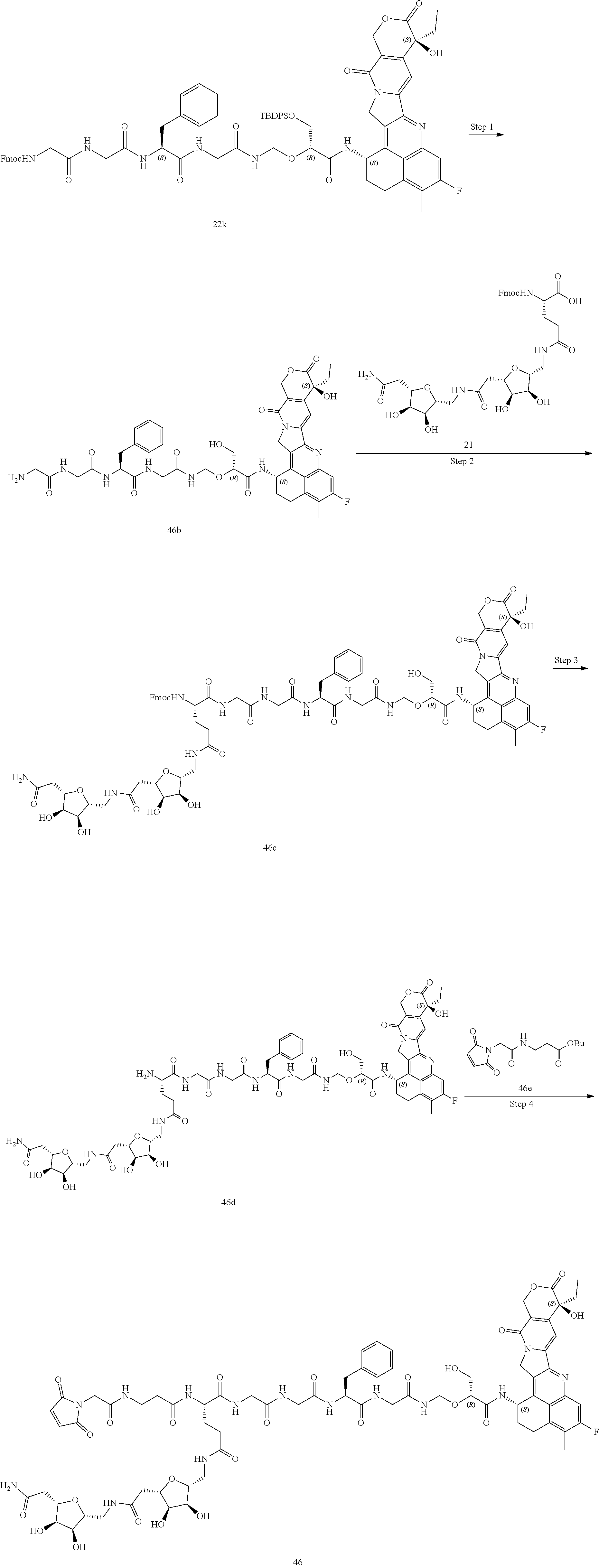

46 (5.6 mg, 44.1% yield) was synthesized using a similar synthetic procedure as that of example 44 (Please refer to example 21 for synthesis of intermediate 21).

MS (ESI) m/z: 1575.9 [M+Na]+

### Example 47

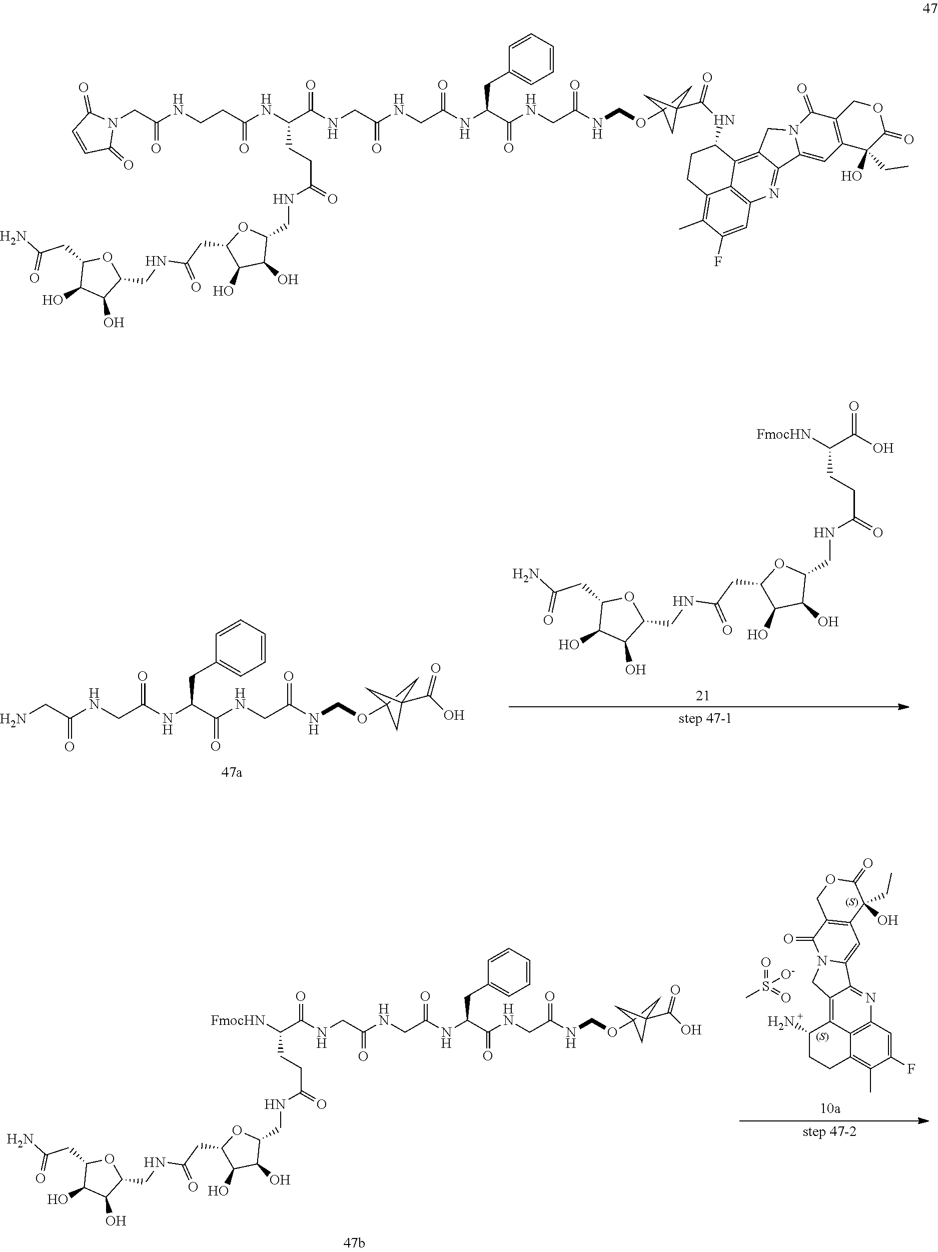

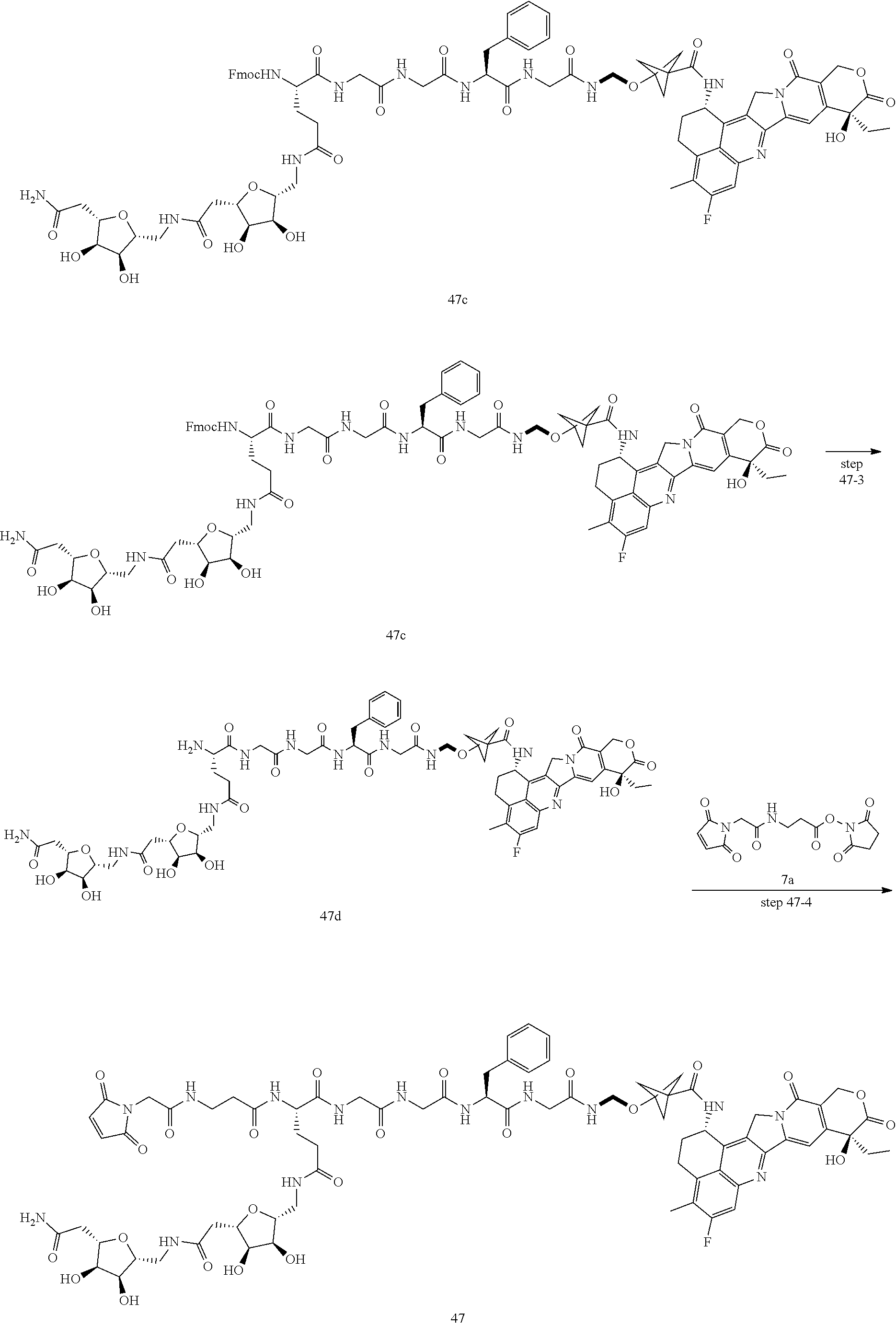

### Step 1

### 3-(((5S,14S)-5-(3-((((2R,3S,4R,5S)-5-(2-((((2R,3S,4R,5S)-5-(2-amino-2-oxoethyl)-3,4-dihydroxytetrahydrofuran-2-yl)methyl)amino)-2-oxoethyl)-3,4-dihydroxytetrahydrofuran-2-yl)methyl)amino)-3-oxopropyl)-14-benzyl-1-(9H-fluoren-9-yl)-3,6,9,12,15,18-hexaoxo-2-oxa-4,7,10,13,16,19-hexaazaicosan-20-yl)oxy)bicyclo[1.1.1]pentane-1-carboxylic acid (38b)

To a mixture of 21 (95.0 mg, 0.133 mmol) and TSTU (40.02 mg, 0.133 mmol) in DMF (2 mL) was added DIPEA (51.54 mg, 0.399 mmol). The mixture was reacted at r.t. for 10 min and the acid was converted to active ester based on LCMS. 47a (385 mg, crude) was added and reacted at the same temperature for another 1 h. On completion of the reaction. The mixture was filtered and the filtrate was purified using prep-HPLC (Method: column: XBridge Prep C18 OBD Sum 19*250 mm; Mobile phase: A—water (0.1% formic acid): B—acetonitrile; Flow rate: 20 mL/min) to provide 47b (66.5 mg, 42.9% yield).

MS (ESI) m/z: 1170.8 [M−H]−

### Step 2

### (9H-fluoren-9-yl)methyl ((7S,16S)-21-((2R,3S,4R,5S)-5-(2-((((2R,3S,4R,5S)-5-(2-amino-2-oxoethyl)-3,4-dihydroxytetrahydrofuran-2-yl)methyl)amino)-2-oxoethyl)-3,4-dihydroxytetrahydrofuran-2-yl)-7-benzyl-1-((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)bicyclo[1.1.1]pentan-1-yl)oxy)-3,6,9,12,15,19-hexaoxo-2,5,8,11,14,20-hexaazahenicosan-16-yl)carbamate (47c)

Compound 47c (29.5 mg, 90.5% yield) was synthesized according to synthetic procedure of step 4 of example 28.

MS (ESI) m/z: 1613.0 [M+Na]+

### Step 3

### (S)-2-amino-N5-(((2R,3S,4R,5S)-5-(2-((((2R,3S,4R,5S)-5-(2-amino-2-oxoethyl)-3,4-dihydroxytetrahydrofuran-2-yl)methyl)amino)-2-oxoethyl)-3,4-dihydroxytetrahydrofuran-2-yl)methyl)-N1-((S)-7-benzyl-1-((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)bicyclo[1.1.1]pentan-1-yl)oxy)-3,6,9,12-tetraoxo-2,5,8,11-tetraazatridecan-13-yl)pentanediamide (47d)

Compound 47d (29.6 mg, crude) was synthesized according to synthetic procedure of step 2 of example 43.

MS (ESI) m/z: 1368.1 [M+H]+

### Step 4

### (S)—N5-(((2R,3S,4R,5S)-5-(2-((((2R,3S,4R,5S)-5-(2-amino-2-oxoethyl)-3,4-dihydroxytetrahydrofuran-2-yl)methyl)amino)-2-oxoethyl)-3,4-dihydroxytetrahydrofuran-2-yl)methyl)-N1-((S)-7-benzyl-1-((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)bicyclo[1.1.1]pentan-1-yl)oxy)-3,6,9,12-tetraoxo-2,5,8,11-tetraazatridecan-13-yl)-2-(3-(2-(2,5-dioxo-2,5-dihydro-1H-pyrrol-1-yl)acetamido)propanamido)pentanediamide (47)

Compound 47 (19.9 mg, 58.3% yield) was synthesized according to synthetic procedure of step 8 of example 2.

MS (ESI) m/z: 1598.9 [M+Na]+

### Example 48

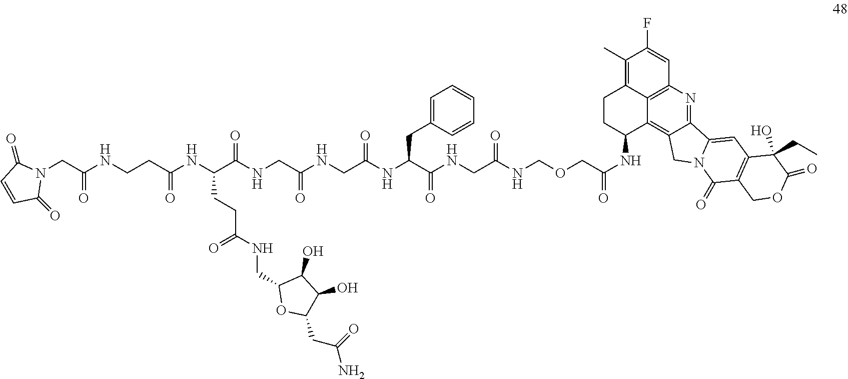 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)[C@H](CCC(=O)NC[C@H]1O[C@@H](CC(N)=O)[C@H](O)[C@@H]1O)NC(=O)CCNC(=O)CN1C(=O)C=CC1=O)CC3]

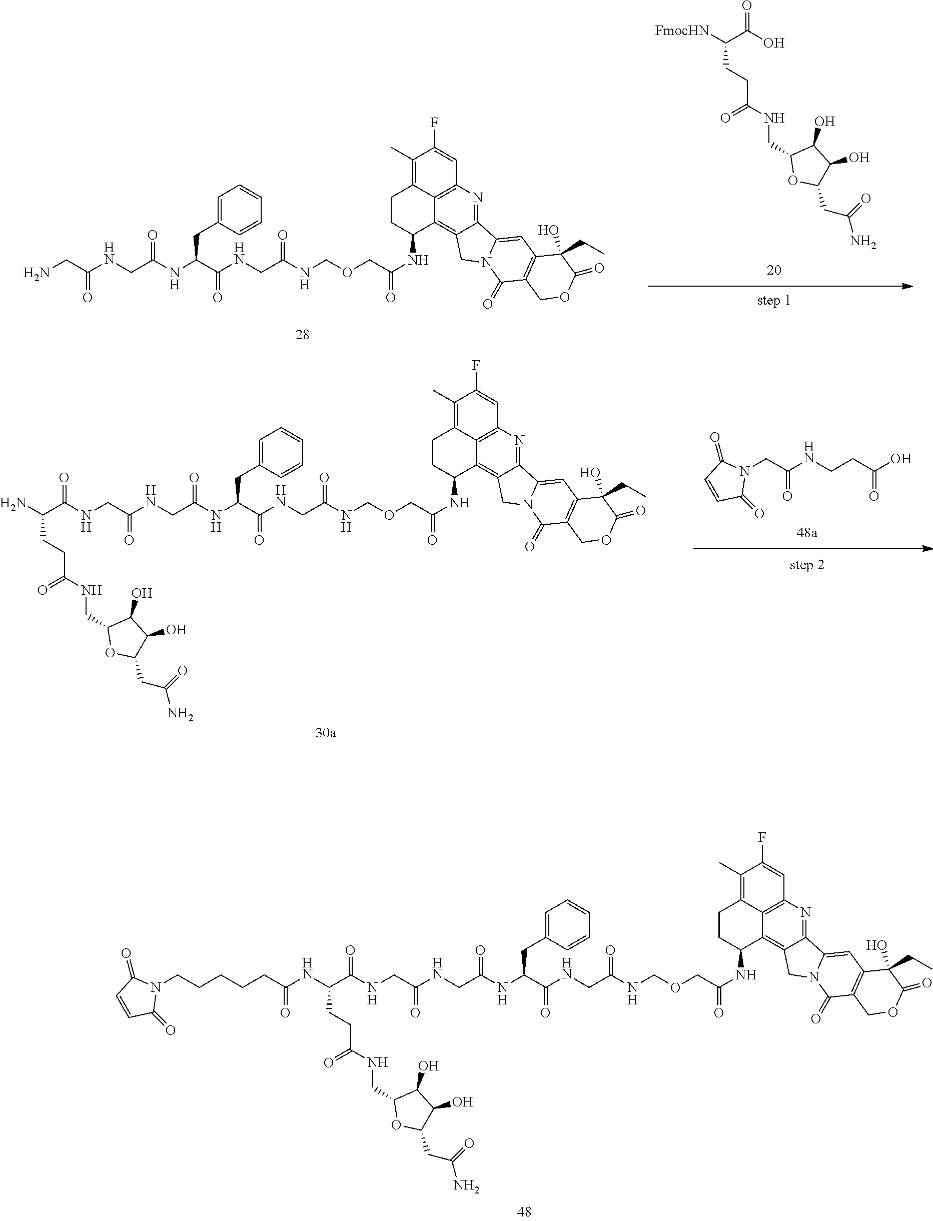

### Step 1

### (S)-2-amino-N5-(((2R,3S,4R,5S)-5-(2-amino-2-oxoethyl)-3,4-dihydroxytetrahydrofuran-2-yl)methyl)-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)pentanediamide (30a)

Compound 30a (20 mg, 36.8% yield) was synthesized according to synthetic procedure of step 1 of example 30.

MS (ESI) m/z: 1142.8 [M+H+]

### Step 2

### (S)-2-amino-N5-(((2R,3S,4R,5S)-5-(2-amino-2-oxoethyl)-3,4-dihydroxytetrahydrofuran-2-yl)methyl)-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)pentanediamide (48)

To a mixture of 48a (5 mg, 0.021 mmol) and HATU (8 mg, 0.021 mmol) in DMF (1 mL) was added DIPEA (5 mg, 0.035 mmol). The mixture was reacted at r.t. for 10 min. 30a (20 mg, 0.018 mmol) was added and reacted at the same temperature for another 15 min. The mixture was filtered and the filtrate was purified using prep-HPLC (Method: column: XBridge Prep C18 OBD Sum 19*250 mm; Mobile phase: A—water (0.1% formic acid): B—acetonitrile; Flow rate: 20 mL/min) to afford 48 (4 mg, 16.9% yield).

MS (ESI) m/z: 1372.8 [M+Na]+

### Example 49

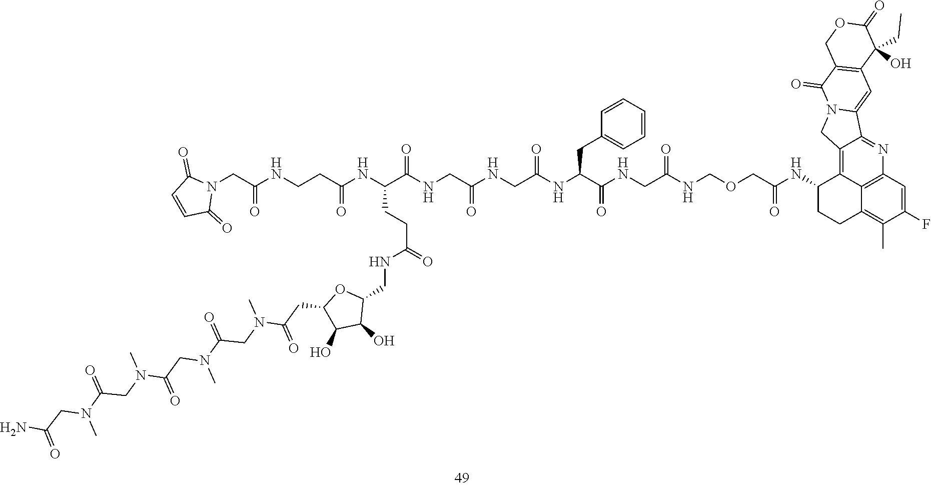 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)[C@H](CCC(=O)NC[C@H]1O[C@@H](CC(=O)N(C)CC(=O)N(C)CC(=O)N(C)CC(=O)N(C)CC(N)=O)[C@H](O)[C@@H]1O)NC(=O)CCNC(=O)CN1C(=O)C=CC1=O)CC3]

[chemical structure]

### Step 1

### 2,5-dioxopyrrolidin-1-yl 2-((3aS,4S,6R,6aR)-2,2-dimethyl-6-((((Z)-2-oxo-2-phenylethylidene)amino)methyl)tetrahydrofuro[3,4-d][1,3]dioxol-4-yl)acetate (49b)

49f (215 mg, theoretical yield) was synthesized according to the synthetic procedure of step 1 of example 39b.

### Step 2

### 14-((3aS,4S,6R,6aR)-6-((((benzyloxy)carbonyl)amino)methyl)-2,2-dimethyltetrahydrofuro[3,4-d][1,3]dioxol-4-yl)-3,6,9,12-tetramethyl-4,7,10,13-tetraoxo-3,6,9,12-tetraazatetradecanoic acid (49d)

To a solution of compound 49c (156 mg, 0.37 mmol) in DMF (3 mL) were added 49b (215.16 mg, 0.47 mmol) and DIEA (289 mg, 2.24 mmol). The mixture was stirred at r.t. for 16 h. The mixture was purified by Prep HPLC (Method: column: XBridge Prep C18 OBD 5 μm 19*250 mm; Mobile phase: A—water (0.1% formic acid): B—acetonitrile; Flow rate: 20 mL/min). Compound 49d (115 mg, 47.3% yield) was obtained as white solid.

MS (ESI) m/z: 650.6 [M+H]+.

### Step 3

### Benzyl (((3aR,4R,6S,6aS)-6-(14-amino-3,6,9,12-tetramethyl-2,5,8,11,14-pentaoxo-3,6,9,12-tetraazatetradecyl)-2,2-dimethyltetrahydrofuro[3,4-d][1,3]dioxol-4-yl)methyl)carbamate (49e)

To a solution of compound 49d (105 mg, 0.16 mmol) in DMF (1 mL) were added PyBOP (127 mg, 0.24 mmol), HOBt (33 mg, 0.24 mmol), DIEA (105 mg, 0.81 mmol) and NH3-MeOH (230 Ul, 7M). The mixture was stirred at r.t. for 1 h. The mixture was purified by Prep HPLC (Method: column: XBridge Prep C18 OBD Sum 19*250 mm; Mobile phase: A—water (0.1% formic acid): B—acetonitrile; Flow rate: 20 mL/min). Compound 49e (87 mg, 83.0% yield) was obtained as white solid.

MS (ESI) m/z: 649.6 [M+H]+.

### Step 4

### N-(2-amino-2-oxoethyl)-2-(2-(2-(2-((3aS,4S,6R,6aR)-6-(aminomethyl)-2,2-dimethyltetrahydrofuro[3,4-d][1,3]dioxol-4-yl)-N-methylacetamido)-N-methylacetamido)-N-methylacetamido)-N-methylacetamide (49f)

49f (85.0 mg, Crude) was synthesized according to the synthetic procedure of step 1 of example 34b.

MS (ESI) m/z: 515.6 [M+H]+.

### Step 5

### Benzyl N2-(((9H-fluoren-9-yl)methoxy)carbonyl)-N5-(((3aR,4R,6S,6aS)-6-(14-amino-3,6,9,12-tetramethyl-2,5,8,11,14-pentaoxo-3,6,9,12-tetraazatetradecyl)-2,2 dimethyltetrahydrofuro[3,4-d][1,3]dioxol-4-yl)methyl)-L-glutaminate (49 h)

To a solution of compound 49g (83.5 mg, 0.18 mmol) in DMF (2 mL) were added HATU (94.2 mg, 0.25 mmol) and DIEA (64 mg, 0.5 mmol). The mixture was stirred at r.t. for 15 min. Compound 49f (85.0 mg, 0.165 mmol) was added into the mixture and stirred at r.t. for 2 h. The mixture was purified by Prep HPLC (Method: column: XBridge Prep C18 OBD Sum 19*250 mm; Mobile phase: A—water (0.1% formic acid): B—acetonitrile; Flow rate: 20 mL/min). Compound 49h (83.0 mg, 52.5% yield) was obtained as white solid.

MS (ESI) m/z: 956.8 [M+H]+.

### Step 6

### Benzyl N2-(((9H-fluoren-9-yl)methoxy)carbonyl)-N5-(((2R,3S,4R,5S)-5-(14-amino-3,6,9,12-tetramethyl-2,5,8,11,14-pentaoxo-3,6,9,12-tetraazatetradecyl)-3,4-dihydroxytetrahydrofuran-2-yl)methyl)-L-glutaminate (49i)

49i (79.0 mg, Crude) was synthesized according to the synthetic procedure of step 3 of example 34e.

MS (ESI) m/z: 916.7 [M+H]+.

### Step 7

### N2-(((9H-fluoren-9-yl)methoxy)carbonyl)-N5-(((2R,3S,4R,5S)-5-(14-amino-3,6,9,12 tetramethyl-2,5,8,11,14-pentaoxo-3,6,9,12-tetraazatetradecyl)-3,4 dihydroxytetrahydrofuran-2-yl)methyl)-L-glutamine (49j)

49j (42.0 mg, 59.2% yield) was synthesized according to the synthetic procedure of step 4 of example 34f.

MS (ESI) m/z: 826.7 [M+H]+.

### Step 8

### (9H-fluoren-9-yl)methyl ((6S,15S)-1-((2R,3S,4R,5S)-5-(14-amino-3,6,9,12-tetramethyl-2,5,8,11,14-pentaoxo-3,6,9,12-tetraazatetradecyl)-3,4-dihydroxytetrahydrofuran-2-yl)-15-benzyl-24-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,24-heptaoxo-22-oxa-2,8,11,14,17,20-hexaazatetracosan-6-yl)carbamate (491)

49l (55.0 mg, theoretical yield) was synthesized according to the synthetic procedure of step 7 of example 34j.

MS (ESI) m/z: 1672.0 [M+Na]+.

### Step 9

### (S)-2-amino-N5-(((2R,3S,4R,5S)-5-(14-amino-3,6,9,12-tetramethyl-2,5,8,11,14-pentaoxo-3,6,9,12-tetraazatetradecyl)-3,4-dihydroxytetrahydrofuran-2-yl)methyl)-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)pentanediamide (49m)

49m (30.28 mg, theoretical yield) was synthesized according to the synthetic procedure of step 8 of example 34k.

MS (ESI) m/z: 1428.1 [M+H]+

### Step 10

### (S)—N5-(((2R,3S,4R,5S)-5-(14-amino-3,6,9,12-tetramethyl-2,5,8,11,14-pentaoxo-3,6,9,12-tetraazatetradecyl)-3,4-dihydroxytetrahydrofuran-2-yl)methyl)-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(3-(2-(2,5-dioxo-2,5-dihydro-1H-pyrrol-1-yl)acetamido)propanamido)pentanediamide (49)

49 (11.2 mg, 31.5% yield) was synthesized according to the synthetic procedure of step 4 of example 39.

MS (ESI) m/z: 1658.0 [M+Na]+

### Example 50

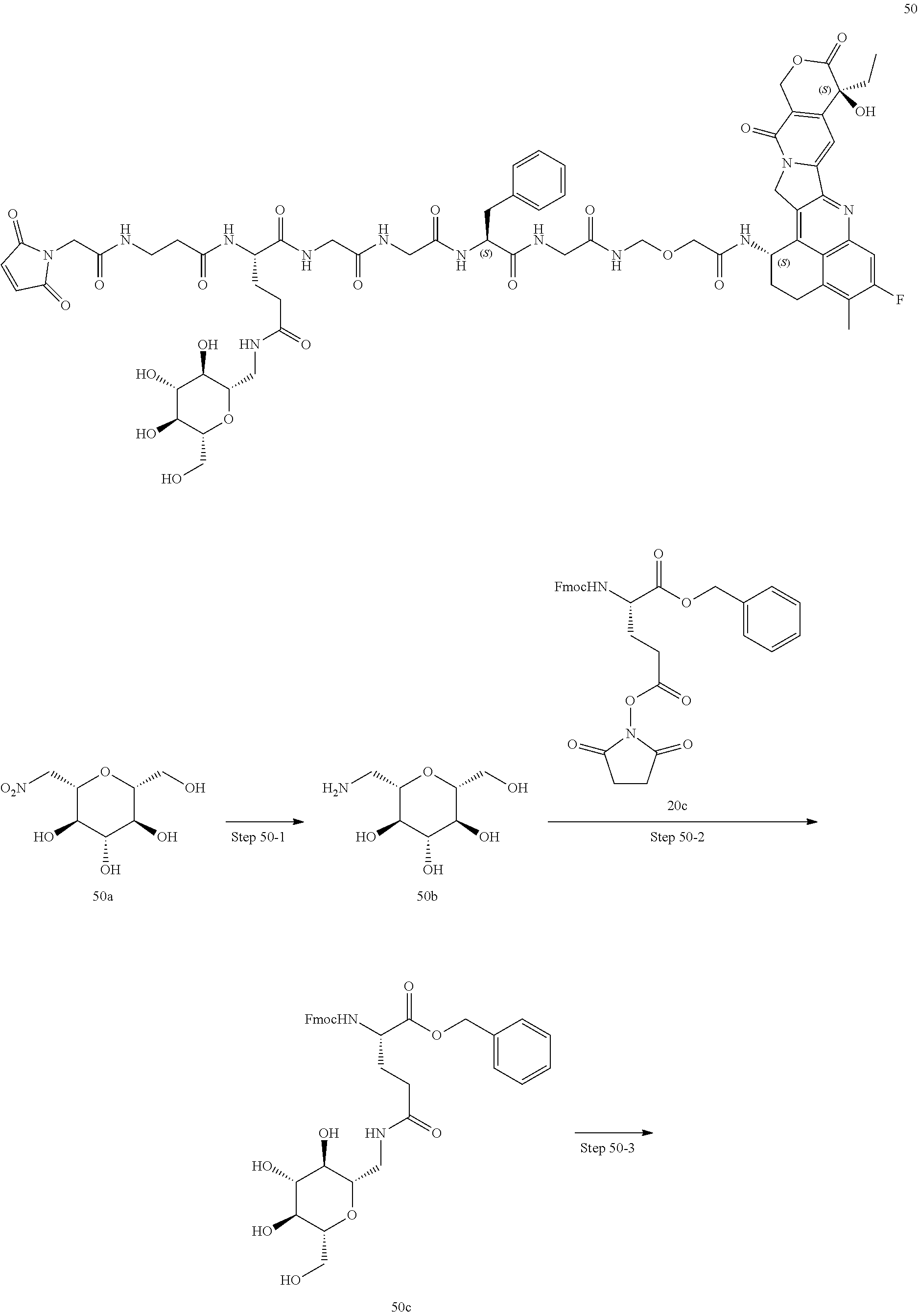

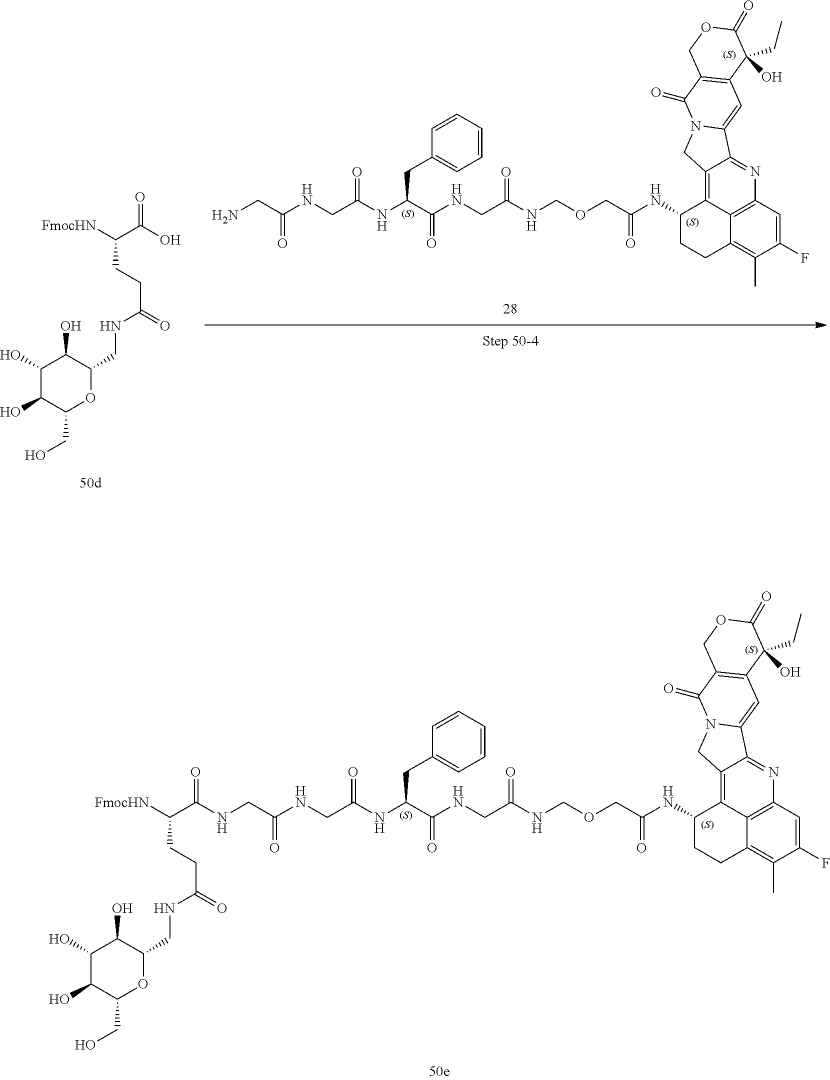

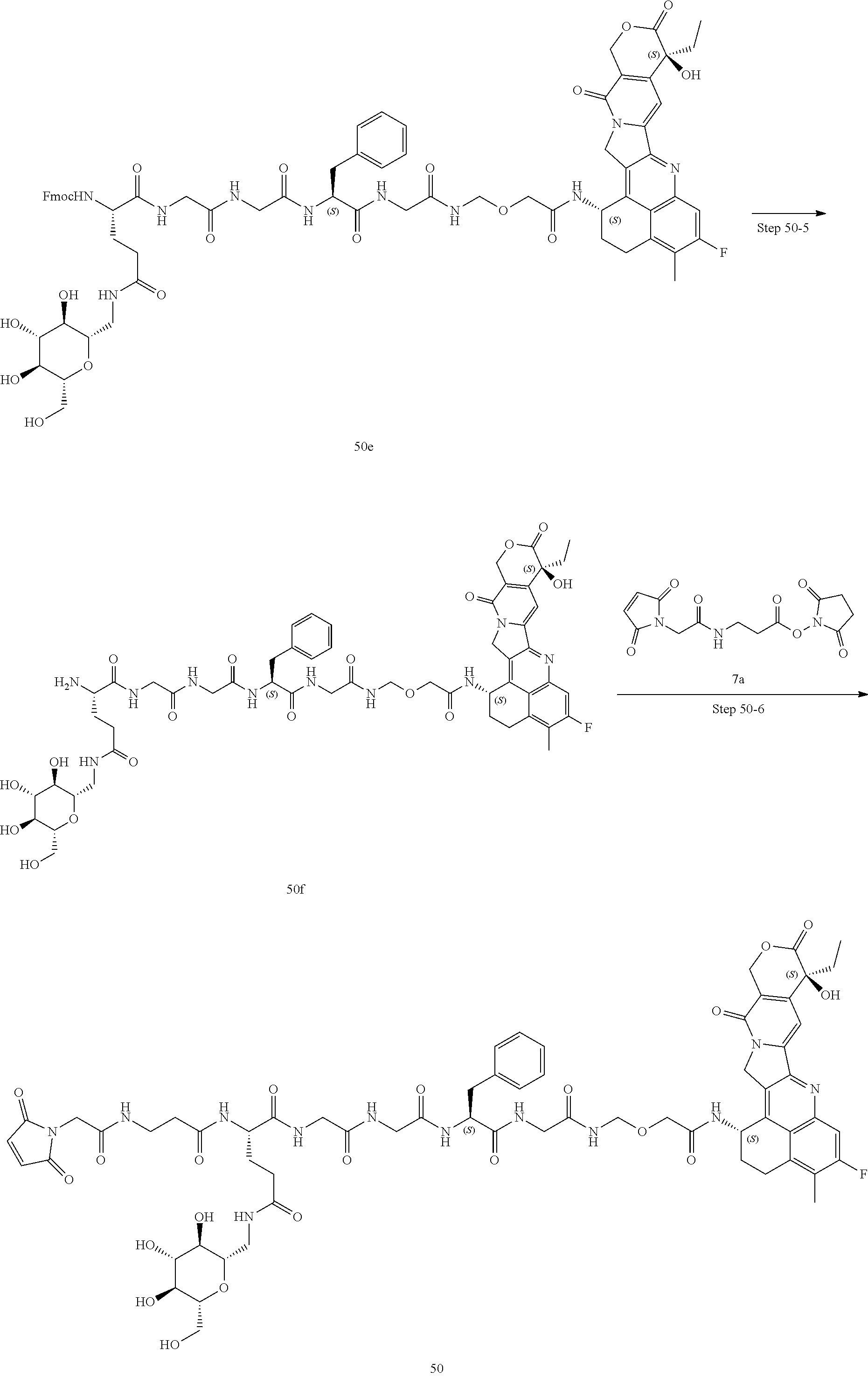

### Step 1

### (2S,3R,4R,5S,6R)-2-(aminomethyl)-6-(hydroxymethyl)tetrahydro-2H-pyran-3,4,5-triol (50b)

To a mixture of 50a (200 mg, 0.896 mmol) in MeOH/H2O (V/V=1:1, 4 mL) was added wet Pd/C (35 mg, 10% purity). The black suspension was purged with H2 balloon for three times and stirred at r.t. for 2 hr. LCMS showed the complete consumption of 50a. The black suspension was filtered through a pad of celite and the filtrate was concentrated to give 50b (173 mg, crude).

MS (ESI) m/z: 194.2 [M+H]+

### Step 2

### Benzyl N2-(((9H-fluoren-9-yl)methoxy)carbonyl)-N5-(((2S,3R,4R,5S,6R)-3,4,5-trihydroxy-6-(hydroxymethyl)tetrahydro-2H-pyran-2-yl)methyl)-L-glutaminate (50c)

To a mixture of 50b (173 mg, crude) and 20c (431.43 mg, 0.775 mmol) in MeOH/H2O (8 mL) was added sat·NaHCO3 (3 mL) and stirred at r.t. for 2 hr. LCMS showed the complete consumption of 50b. The mixture was concentrated under vacuum to removed most of MeOH and the aqueous phase was acidified with 1N HCl to pH=6, filtered and the filtrate was purified by prep-HPLC (FA) column: XBridge Prep C18 OBD Sum 19*250 mm; Mobile phase: A—water (0.1% formic acid): B—acetonitrile; Flow rate: 20 mL/min) to provide 50c (35 mg, 7.1% yield).

MS (ESI) m/z: 635.5 [M+H]+

### Step 3

### N2-(((9H-fluoren-9-yl)methoxy)carbonyl)-N5-(((2S,3R,4R,5S,6R)-3,4,5-trihydroxy-6-(hydroxymethyl)tetrahydro-2H-pyran-2-yl)methyl)-L-glutamine (50d)

Compound 50d (30 mg, crude) was synthesized according to synthetic procedure of step 2 of example 19.

MS (ESI) m/z: 545.5 [M+H]+

### Step 4

### (9H-fluoren-9-yl)methyl ((6S,15S)-15-benzyl-24-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,24-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazatetracosan-6-yl)carbamate (50e)

Compound 50e (32.5 mg, 43.1% yield) was synthesized according to synthetic procedure of step 1 of example 38.

MS (ESI) m/z: 1389.9 [M+Na]+

### Step 5

### (S)-2-amino-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-(((2S,3R,4R,5S,6R)-3,4,5-trihydroxy-6-(hydroxymethyl)tetrahydro-2H-pyran-2-yl)methyl)pentanediamide (50f)

Compound 50f (29.5 mg, crude) was synthesized according to synthetic procedure of step 3 of example 42.

MS (ESI) m/z: 1145.8 [M+H]+

### Step 6

### (S)—N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(3-(2-(2,5-dioxo-2,5-dihydro-1H-pyrrol-1-yl)acetamido)propanamido)-N5-(((2S,3R,4R,5S,6R)-3,4,5-trihydroxy-6-(hydroxymethyl)tetrahydro-2H-pyran-2-yl)methyl)pentanediamide (50)

Compound 50 (9.5 mg, 34.2% yield) was synthesized according to synthetic procedure of step 8 of example 2.

MS (ESI) m/z: 1354.4 [M+H]+

### Example 51

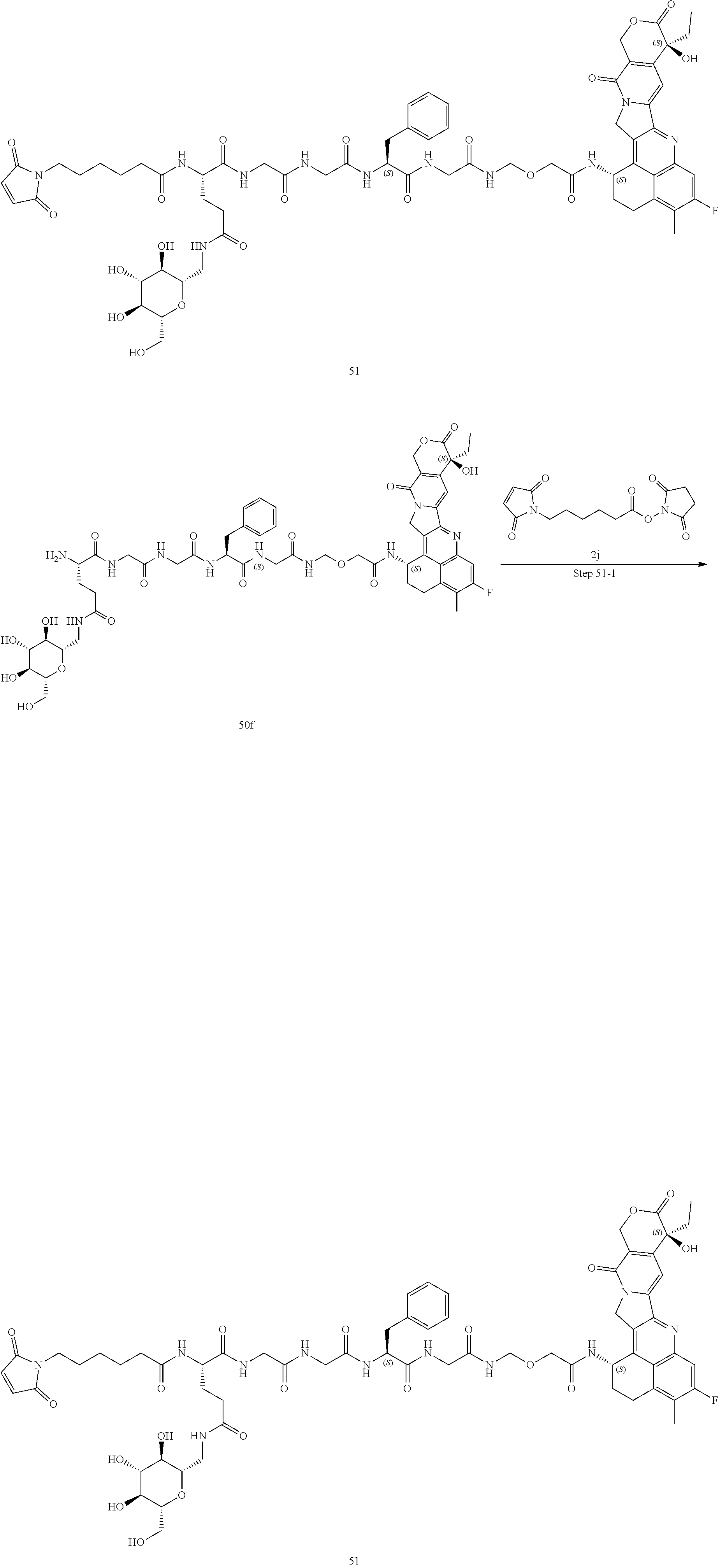

### Step 1

### (S)—N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-2-(6-(2,5-dioxo-2,5-dihydro-1H-pyrrol-1-yl)hexanamido)-N5-(((2 S,3R,4R,5S,6R)-3,4,5-trihydroxy-6-(hydroxymethyl)tetrahydro-2H-pyran-2-yl)methyl)pentanediamide (51)

Compound 51 (24.2 mg, 34.5% yield) was synthesized according to synthetic procedure of step 8 of example 2.

MS (ESI) m/z: 1339.2 [M+H]+

### Example 52

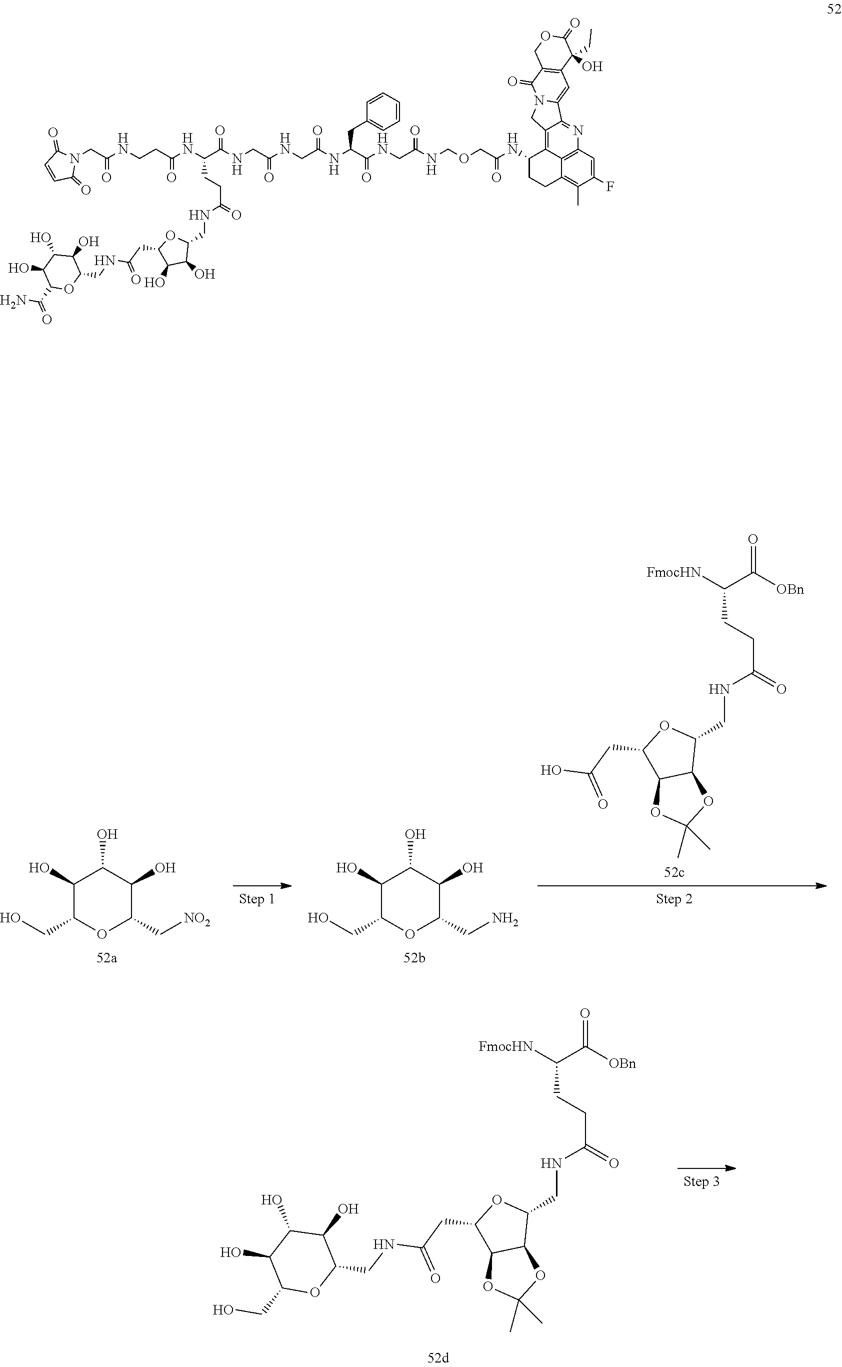

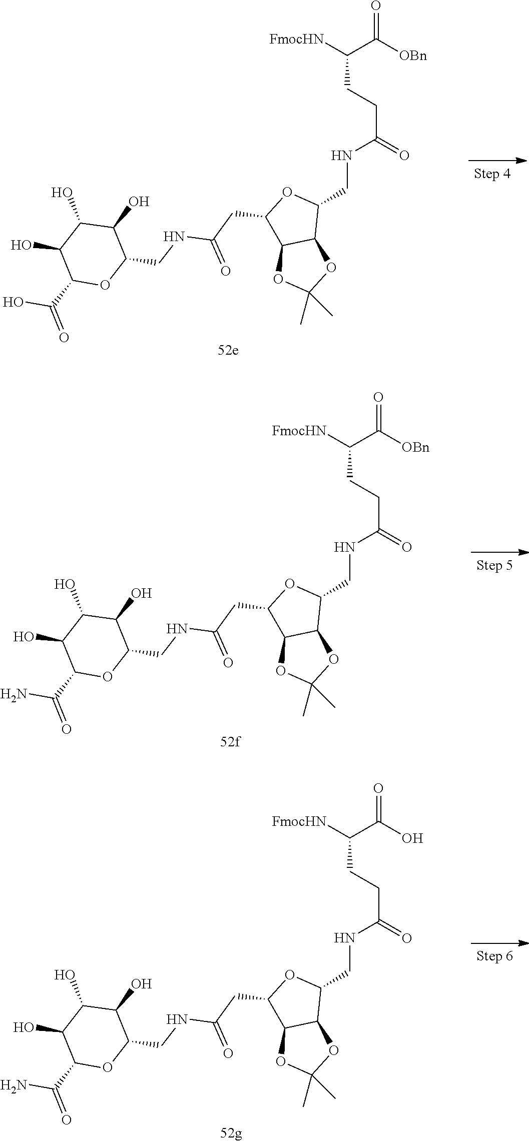

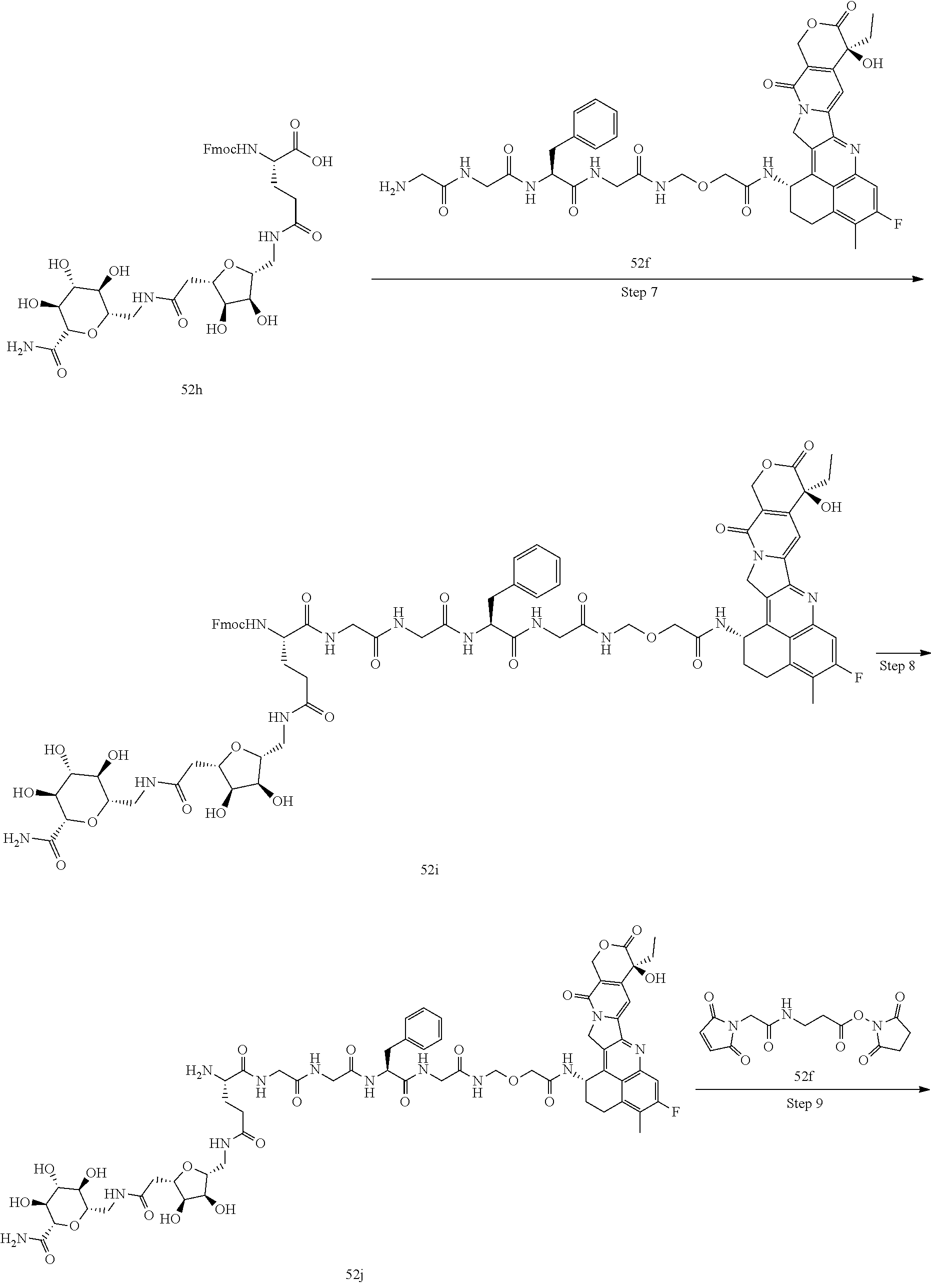

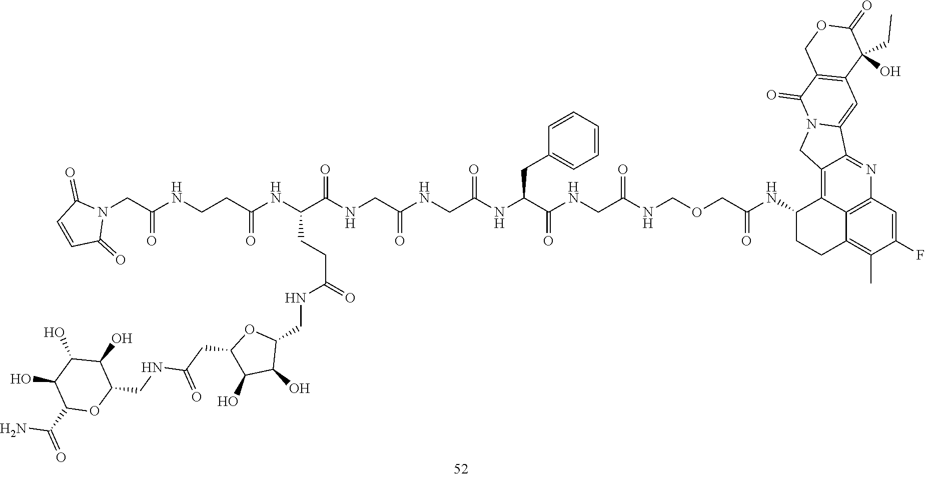

### Step 1

### (2S,3R,4R,5S,6R)-2-(aminomethyl)-6-(hydroxymethyl)tetrahydro-2H-pyran-3,4,5-triol (52b)

To a solution of compound 52a (200 mg, 0.89 mmol) in MeOH (3 mL) were added HCOONH4 (226 mg, 3.58 mmol) and Pd/C (20 mg, 10%). The mixture was stirred at 68° C. for 1 h. H2O (10 mL) was added into the mixture and filtered. The filtrate was concentrated to give the crude product. Compound 52b (210 mg, crude) was obtained as white solid.

MS (ESI) m/z: 194.3 [M+H]+.

### Step 2

### Benzyl N2-(((9H-fluoren-9-yl)methoxy)carbonyl)-N5-(((3aR,4R,6S,6aS)-2,2-dimethyl-6-(2-oxo-2-((((2S,3R,4R,5S,6R)-3,4,5-trihydroxy-6-(hydroxymethyl)tetrahydro-2H-pyran-2-yl)methyl)amino)ethyl)tetrahydrofuro[3,4-d][1,3]dioxol-4-yl)methyl)-L-glutaminate (52d)

52d (152 mg, 48.2% yield) was synthesized according to the synthetic procedure of step 1 of example 45a.

MS (ESI) m/z: 848.7 [M+H]+.

### Step 3

### (2S,3S,4R,5R,6S)-6-((2-((3aR,4R,6S,6aS)-6-(((S)-4-((((9H-fluoren-9-yl)methoxy)carbonyl)amino)-5-(benzyloxy)-5-oxopentanamido)methyl)-2,2-dimethyltetrahydrofuro[3,4-d][1,3]dioxol-4-yl)acetamido)methyl)-3,4,5-trihydroxytetrahydro-2H-pyran-2-carboxylic acid (52e)

To a solution of compound 52d (152 mg, 0.18 mmol) in THE (4 mL) and H2O (2 mL) were added TEMPO (2.8 mg, 0.018 mmol), KBr (2.1 mg, 0.018 mmol) and NaHCO3 (151 mg, 1.79 mmol). NaClO (953 mg, 0.90 mmol, 7%) was added into the mixture dropwise at 0° C. The mixture was stirred at r.t. for 1 h. The mixture was adjusted to pH 5. The mixture was purified using prep-HPLC (Method: column: XBridge Prep C18 OBD 5 μm 19*250 mm; Mobile phase: A—water (0.1% formic acid): B—acetonitrile; Flow rate: 20 mL/min). Compound 52e (115 mg, 74.7% yield) was obtained as white solid.

MS (ESI) m/z: 862.6 [M+Na]+.

### Step 4

### Benzyl N2-(((9H-fluoren-9-yl)methoxy)carbonyl)-N5-(((3aR,4R,6S,6aS)-6-(2-((((2S,3R,4R,5S,6S)-6-carbamoyl-3,4,5-trihydroxytetrahydro-2H-pyran-2-yl)methyl)amino)-2-oxoethyl)-2,2-dimethyltetrahydrofuro[3,4-d][1,3]dioxol-4-yl)methyl)-L-glutaminate (52f)

52f (52 mg, 45.2% yield) was synthesized according to the synthetic procedure of step 3 of example 49e.

MS (ESI) m/z: 861.7 [M+H]+.

### Step 5

### N2-(((9H-fluoren-9-yl)methoxy)carbonyl)-N5-(((3aR,4R,6S,6aS)-6-(2-((((2S,3R,4R,5S,6S)-6-carbamoyl-3,4,5-trihydroxytetrahydro-2H-pyran-2-yl)methyl)amino)-2-oxoethyl)-2,2-dimethyltetrahydrofuro[3,4-d][1,3]dioxol-4-yl)methyl)-L-glutamine (52 g)

52 g (45 mg, crude) was synthesized according to the synthetic procedure of step 7 of example 49j.

MS (ESI) m/z: 771.6 [M+H]+.

### Step 6

### N2-(((9H-fluoren-9-yl)methoxy)carbonyl)-N5-(((3aR,4R,6S,6aS)-6-(2-((((2S,3R,4R,5S,6S)-6-carbamoyl-3,4,5-trihydroxytetrahydro-2H-pyran-2-yl)methyl)amino)-2-oxoethyl)-2,2-dimethyltetrahydrofuro[3,4-d][1,3]dioxol-4-yl)methyl)-L-glutamine (52 h)

52 h (32 mg, 71.7% yield) was synthesized according to the synthetic procedure of step 6 of example 49i.

MS (ESI) m/z: 731.5 [M+H]+.

### Step 7

### N2-(((9H-fluoren-9-yl)methoxy)carbonyl)-N5-(((2R,3S,4R,5S)-5-(2-((((2S,3R,4R,5S,6S)-6-carbamoyl-3,4,5-trihydroxytetrahydro-2H-pyran-2-yl)methyl)amino)-2-oxoethyl)-3,4-dihydroxytetrahydrofuran-2-yl)methyl)-L-glutamine (52i)

52i (24 mg, 35.3% yield) was synthesized according to the synthetic procedure of step 8 of example 491.

MS (ESI) m/z: 1575.9 [M+Na]+.

### Step 8

### (9H-fluoren-9-yl)methyl ((6S,15S)-15-benzyl-1-((2R,3S,4R,5S)-5-(2-((((2S,3R,4R,5S,6S)-6-carbamoyl-3,4,5-trihydroxytetrahydro-2H-pyran-2-yl)methyl)amino)-2-oxoethyl)-3,4-dihydroxytetrahydrofuran-2-yl)-24-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,24-heptaoxo-22-oxa-2,8,11,14,17,20-hexaazatetracosan-6-yl)carbamate (52j)

52j (20.6 mg, theoretical yield) was synthesized according to the synthetic procedure of step 9 of example 49m.

MS (ESI) m/z: 1354.8 [M+Na]+.

### Step 9

### (S)-2-amino-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-(((2R,3 S,4R,5S)-5-(2-((((2S,3R,4R,5S,6S)-6-carbamoyl-3,4,5-trihydroxytetrahydro-2H-pyran-2-yl)methyl)amino)-2-oxoethyl)-3,4-dihydroxytetrahydrofuran-2-yl)methyl)pentanediamide (52)

52 (7.8 mg, 32.7% yield) was synthesized according to the synthetic procedure of step 8 of example 34k.

MS (ESI) m/z: 1561.9 [M+Na]+

### Example 53

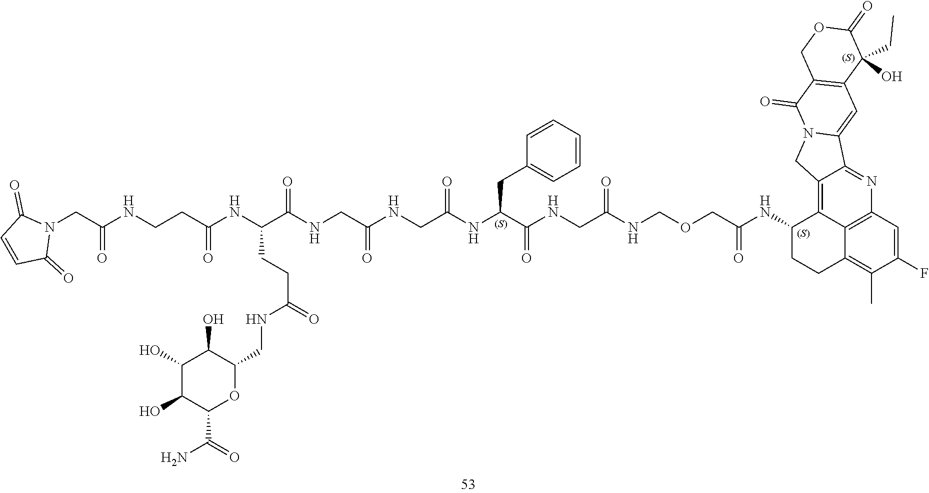

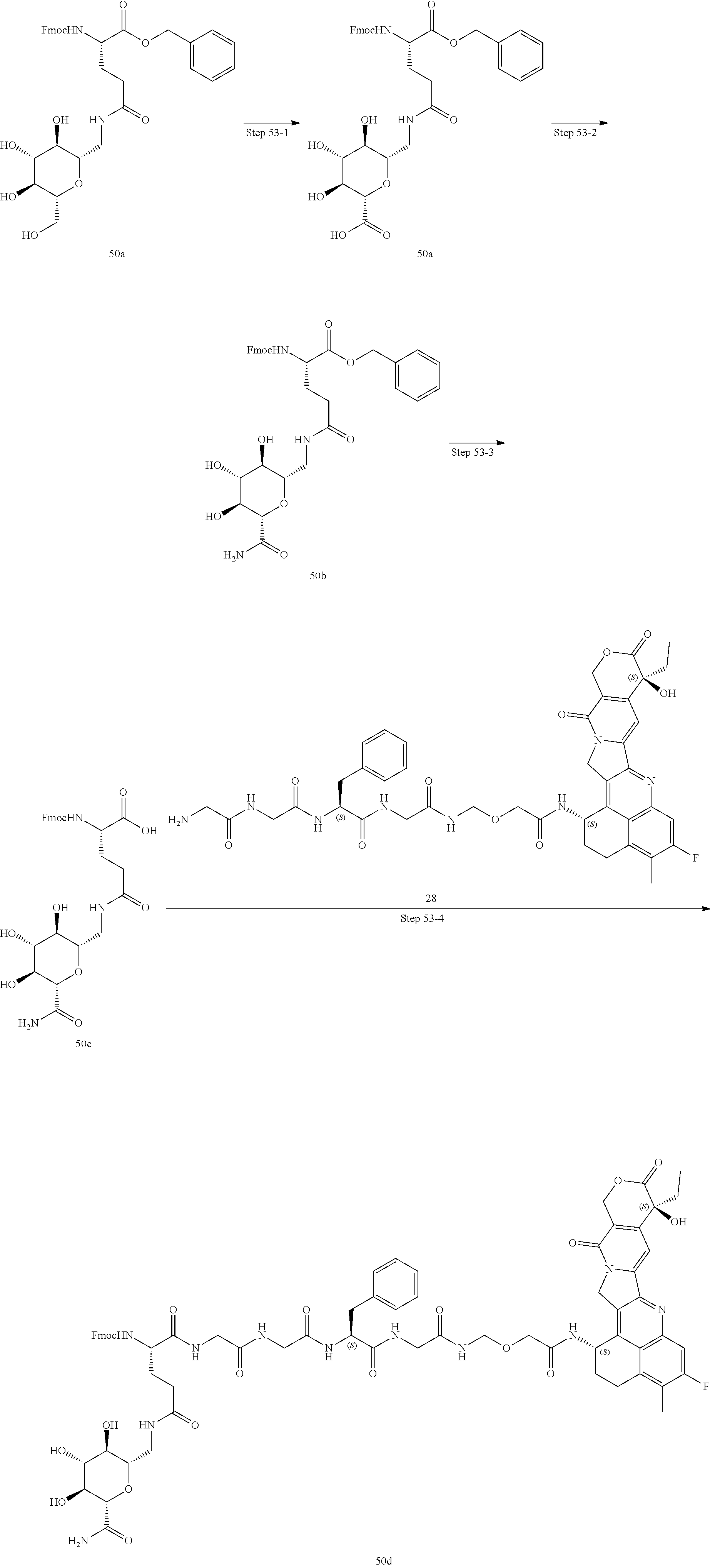

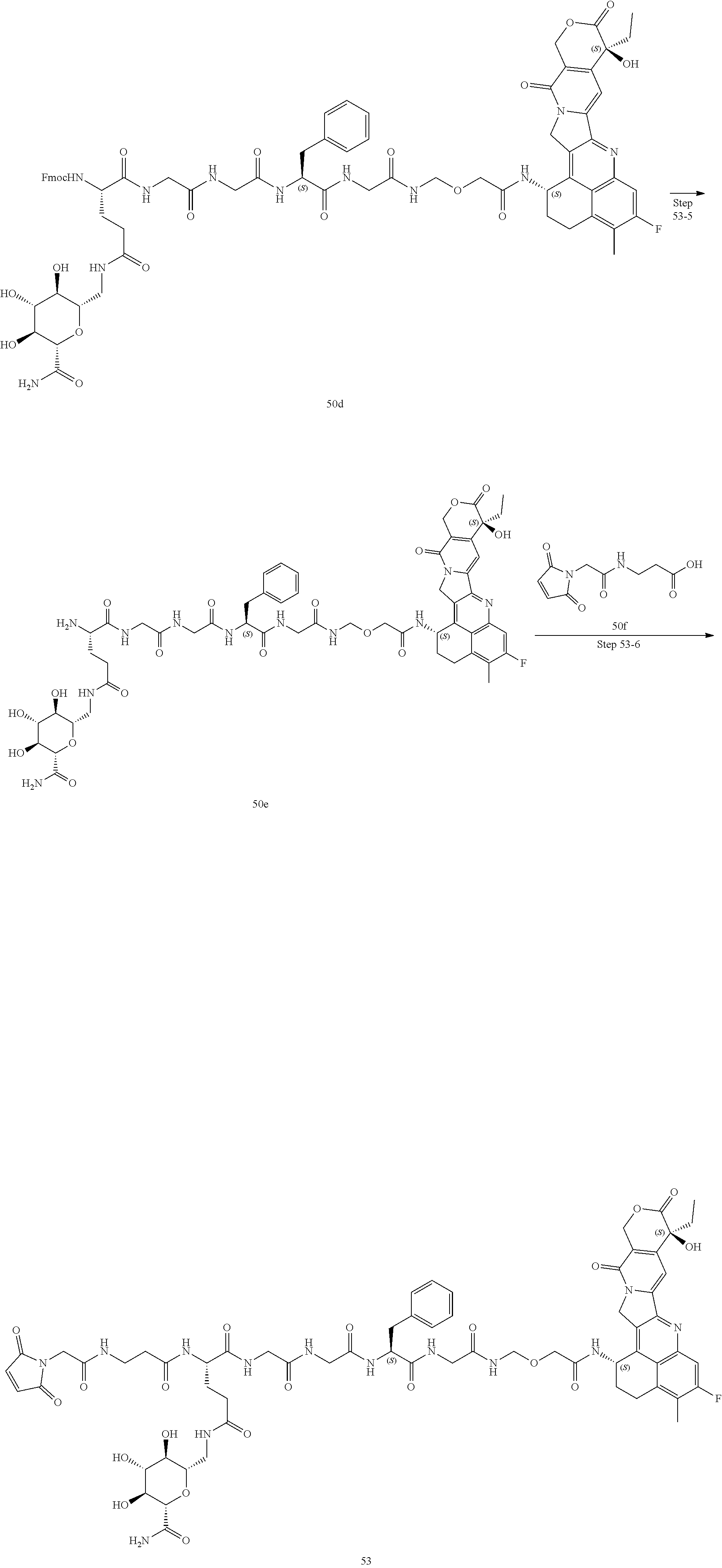

### Step 1

### (2S,3S,4R,5R,6S)-6-(((S)-4-((((9H-fluoren-9-yl)methoxy)carbonyl)amino)-5-(benzyloxy)-5-oxopentanamido)methyl)-3,4,5-trihydroxytetrahydro-2H-pyran-2-carboxylic acid (53a)

To a mixture of 50c (200 mg, 0.32 mmol) in THE (1 mL) and water (1 mL), was added the sodium bicarbonate (158.84 mg, 1.89 mmol). To the mixture was added TEMPO (9.85 mg, 0.06 mmol) and Potassium bromide (11.25 mg, 0.09 mmol). The mixture was cooled to 0° C. with ice bath, sodium hypochrolite solution (aq, 3%˜6% chorine)(1.03 g, 0.69 mmol) dropwise. After addition, the reaction mixture was concentrated under vacuum without heating to remove organic volatiles. The aqueous layer was acidified with 1N HCl until pH reached 2, filtered and the filtrate was purified by prep-HPLC (FA) column: XBridge Prep C18 OBD Sum 19*250 mm; Mobile phase: A—water (0.1% formic acid): B—acetonitrile; Flow rate: 20 mL/min) to provide 53a (137.7 mg, 67% yield).

MS (ESI) m/z: 649.8 [M+H]+

### Step 2

### Benzyl N2-(((9H-fluoren-9-yl)methoxy)carbonyl)-N5-(((2S,3R,4R,5S,6S)-6-carbamoyl-3,4,5-trihydroxytetrahydro-2H-pyran-2-yl)methyl)-L-glutaminate (53b)

To a mixture of 53a (137.7 mg, 0.21 mmol), PyBOP (113.78 mg, 0.22 mmol) and HOBt (29.55 mg, 0.22 mmol) in DMF (1 mL) were added DIPEA (27.44 mg, 0.21 mmol) and NH3·MeOH (7M, 0.42 mmol, 60 uL). The resulted yellow mixture was reacted at r.t. for 1 hr. LCMS showed the complete consumption of 53b. The mixture was purified by prep-HPLC (FA) column: XBridge Prep C18 OBD 5 μm 19*250 mm; Mobile phase: A—water (0.1% formic acid): B—acetonitrile; Flow rate: 20 mL/min) to provide 53b (61.7 mg, 45% yield).

MS (ESI) m/z: 648.5 [M+H]+

### Step 3

### N2-(((9H-fluoren-9-yl)methoxy)carbonyl)-N5-(((2S,3R,4R,5S,6S)-6-carbamoyl-3,4,5-trihydroxytetrahydro-2H-pyran-2-yl)methyl)-L-glutamine (53c)

Compound 53c (30 mg, crude) was synthesized according to synthetic procedure of step 2 of example 19.

MS (ESI) m/z: 558.5 [M+H]+

### Step 4

### (9H-fluoren-9-yl)methyl ((6S,15S)-15-benzyl-1-((2S,3R,4R,5S,6S)-6-carbamoyl-3,4,5-trihydroxytetrahydro-2H-pyran-2-yl)-24-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,24-heptaoxo-22-oxa-2,8,11,14,17,20-hexaazatetracosan-6-yl)carbamate (53d)

Compound 53d (66.7 mg, 50.9% yield) was synthesized according to synthetic procedure of step 1 of example 38.

MS (ESI) m/z: 1402.8 [M+Na]+

### Step 5

### (S)-2-amino-N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-(((2S,3R,4R,5S,6S)-6-carbamoyl-3,4,5-trihydroxytetrahydro-2H-pyran-2-yl)methyl)pentanediamide (53e)

Compound 53e (55.5 mg, crude) was synthesized according to synthetic procedure of step 3 of example 42.

MS (ESI) m/z: 1158.9 [M+H]+

### Step 6

### (S)—N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-(((2S,3R,4R,5S,6S)-6-carbamoyl-3,4,5-trihydroxytetrahydro-2H-pyran-2-yl)methyl)-2-(3-(2-(2,5-dioxo-2,5-dihydro-1H-pyrrol-1-yl)acetamido)propanamido)pentanediamide (53)

To a mixture of 53f (7.91 mg, 0.035 mmol) and HATU (13.30 mg, 0.035 mmol) in DMF (2 mL) was added DIPEA (3.01 mg, 0.023 mmol). The mixture was reacted at r.t. for 5 min. 53e (27.7 mg, crude) was added and reacted at r.t. for 10 min. LCMS showed the complete consumption of 53e. The mixture was purified by prep-HPLC (FA) column: XBridge Prep C18 OBD Sum 19*250 mm; Mobile phase: A—water (0.1% formic acid): B—acetonitrile; Flow rate: 20 mL/min) to provide 53 (23.9 mg, 50.0% yield).

MS (ESI) m/z: 1367.1 [M+H]+

### Example 54

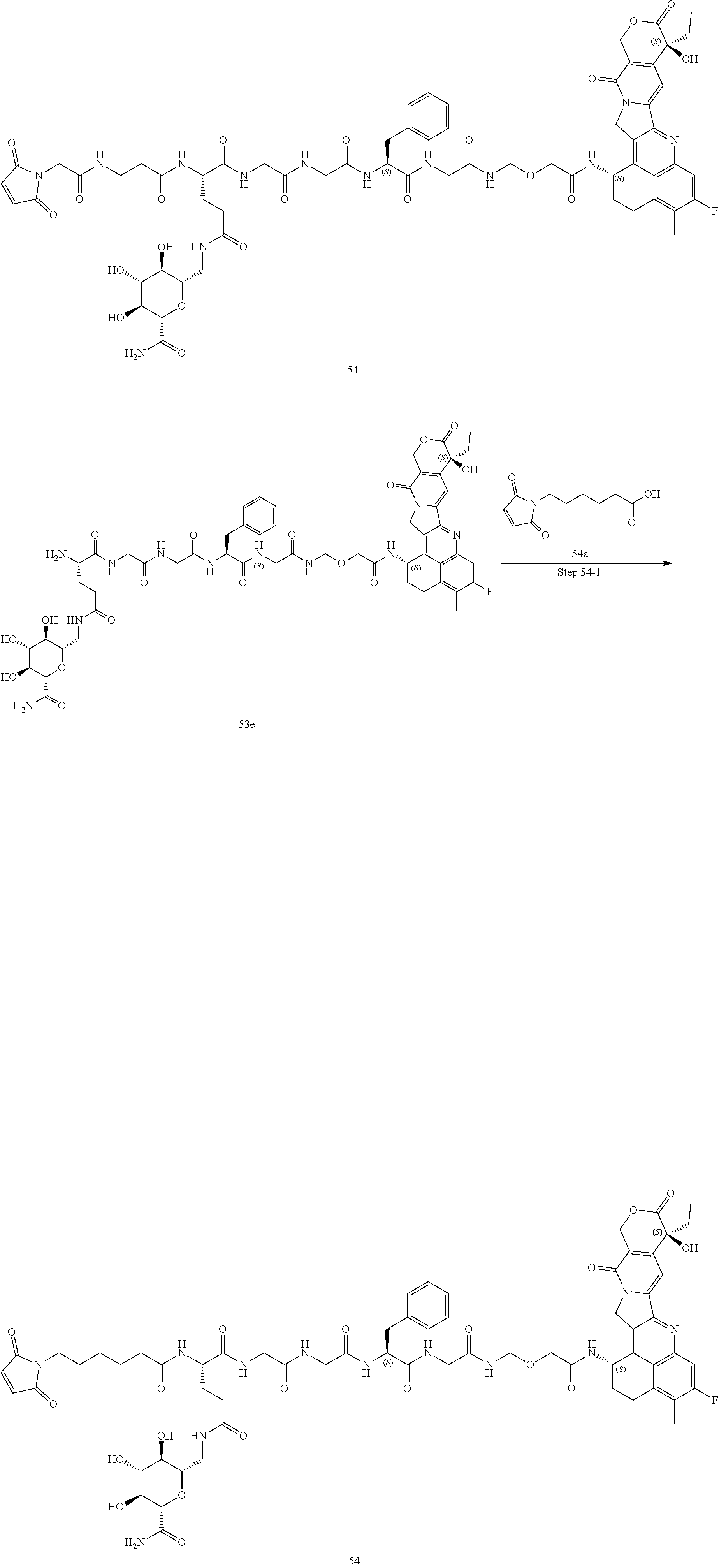

### (S)—N1-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-N5-(((2S,3R,4R,5S,6S)-6-carbamoyl-3,4,5-trihydroxytetrahydro-2H-pyran-2-yl)methyl)-2-(3-(2-(2,5-dioxo-2,5-dihydro-1H-pyrrol-1-yl)acetamido)propanamido)pentanediamide (54)

Compound 54 (21.5 mg, 45.5% yield) was synthesized according to synthetic procedure of step 6 of example 53.

MS (ESI) m/z: 1352.3 [M+H]+

### Example 55

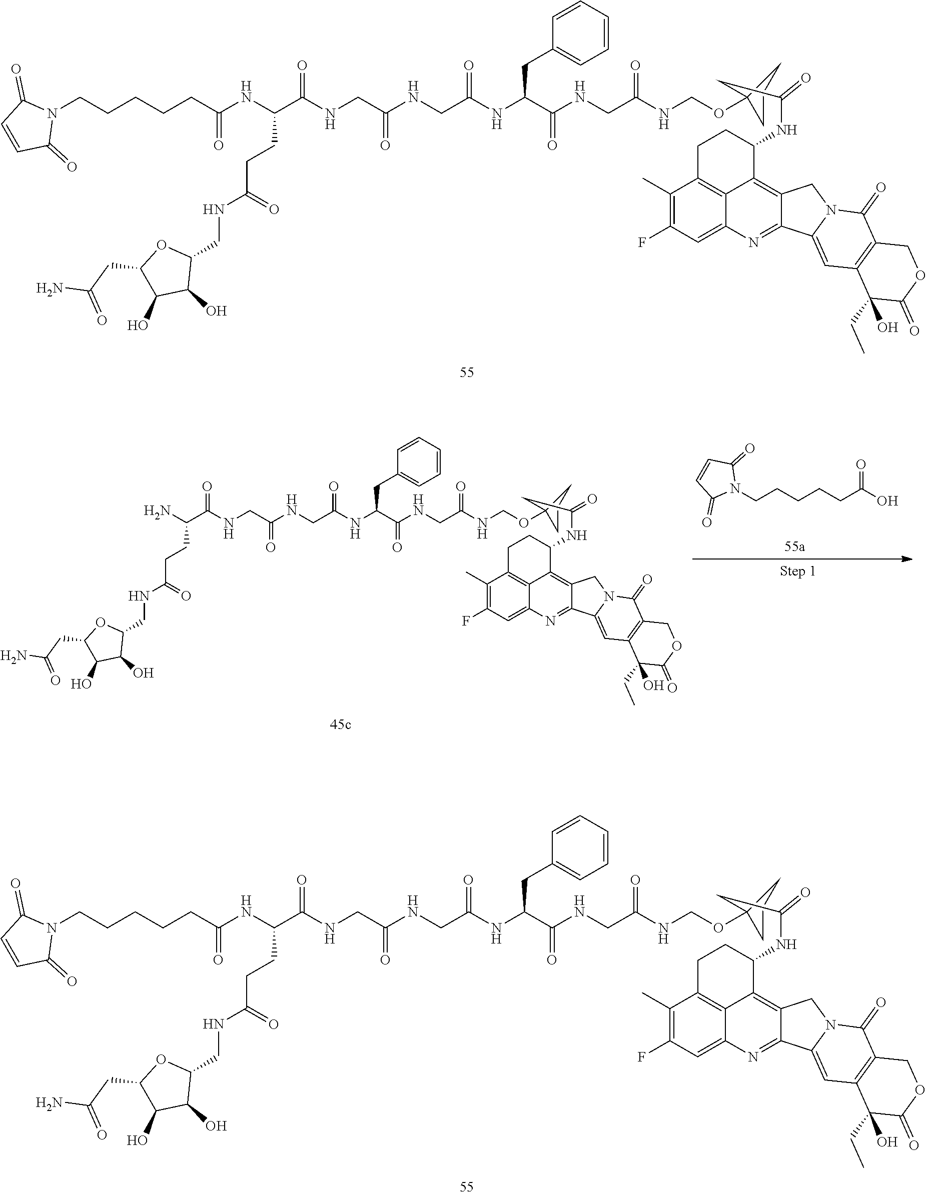

### (S)—N5-(((2R,3S,4R,5S)-5-(2-amino-2-oxoethyl)-3,4-dihydroxytetrahydrofuran-2-yl)methyl)-N1-((S)-7-benzyl-1-((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)bicyclo[1.1.1]pentan-1-yl)oxy)-3,6,9,12-tetraoxo-2,5,8,11-tetraazatridecan-13-yl)-2-(6-(2,5-dioxo-2,5-dihydro-1H-pyrrol-1-yl)hexanamido)pentanediamide (55)

55 (12.5 mg, 59.8% yield) was synthesized according to the synthetic procedure of step 9 of example 34.

MS (ESI) m/z: 1388.3 [M+H]+

### Example 56

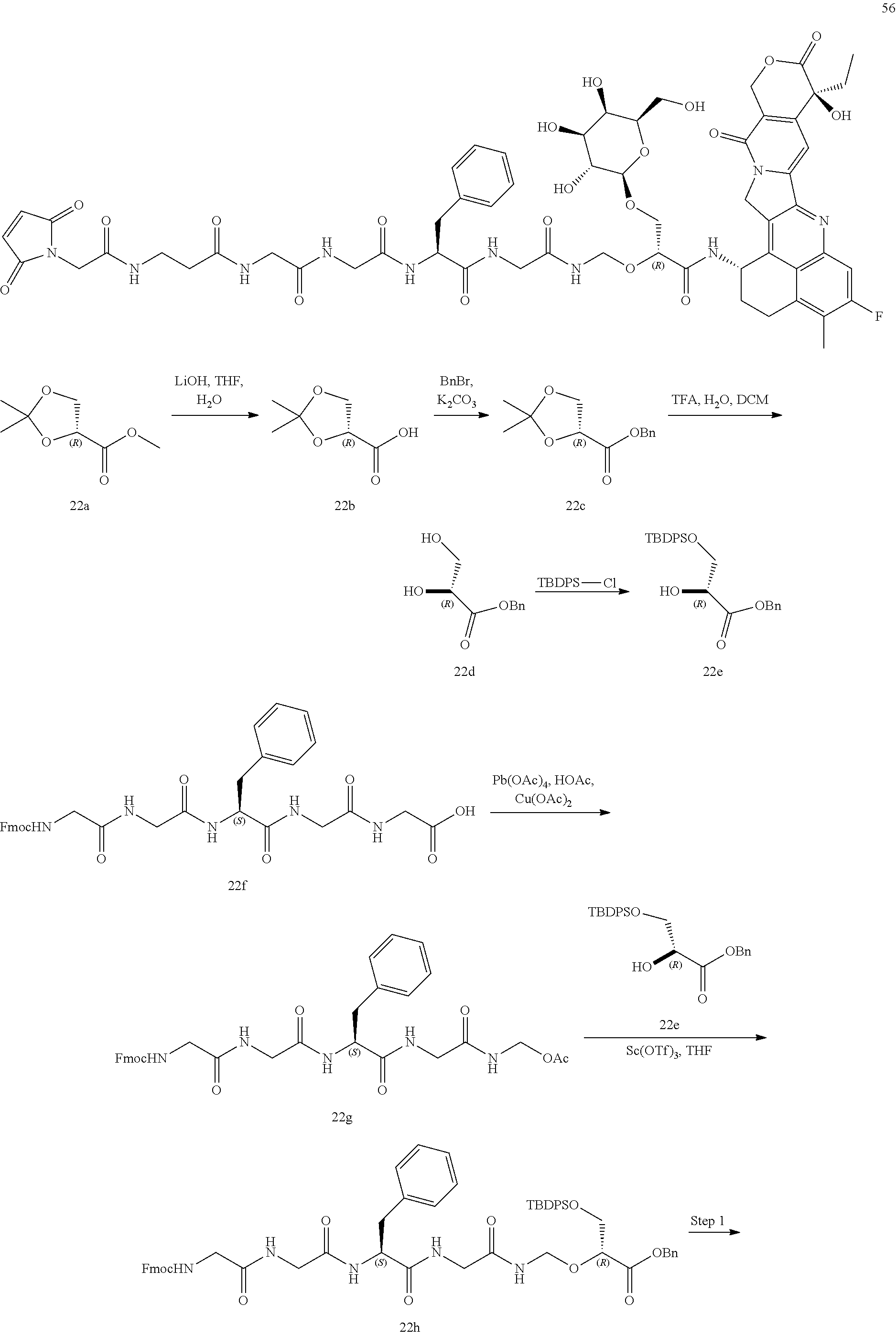

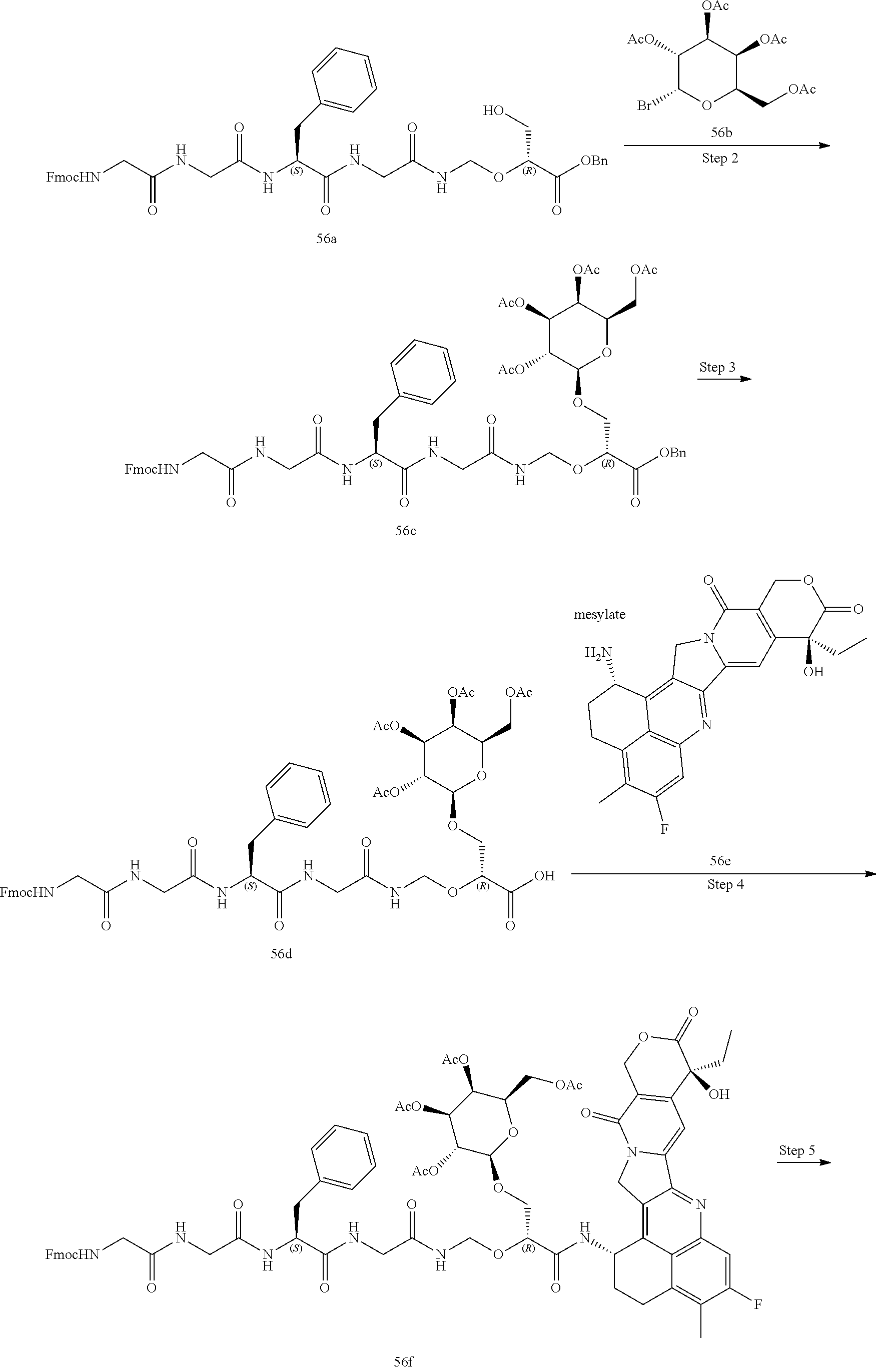

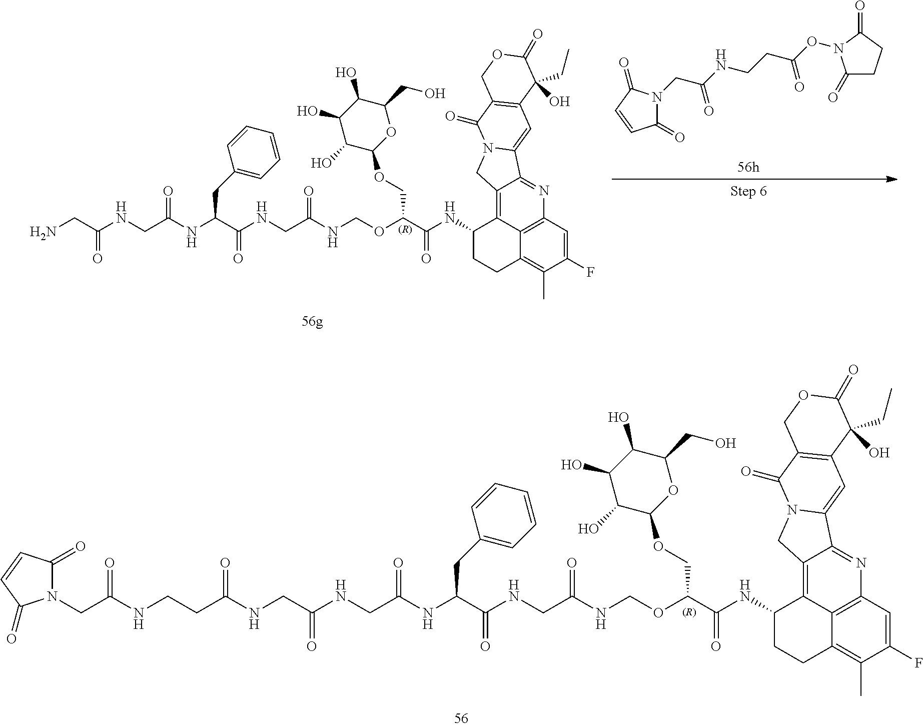

### Benzyl (11S,19R)-11-benzyl-19-(((tert-butyldiphenylsilyl)oxy)methyl)-1-(9H-fluoren-9-yl)-3,6,9,12,15-pentaoxo-2,18-dioxa-4,7,10,13,16-pentaazaicosan-20-oate (22 h)

Please refer to example 22 for synthesis of 22 h.

MS (ESI) m/z: 1026.5 [M+Na]+

### Step 1

### (2R,3S,4S,5R,6R)-2-(acetoxymethyl)-6-(((11S,19R)-11-benzyl-19-((benzyloxy)carbonyl)-1-(9H-fluoren-9-yl)-3,6,9,12,15-pentaoxo-2,18-dioxa-4,7,10,13,16-pentaazaicosan-20-yl)oxy)tetrahydro-2H-pyran-3,4,5-triyl triacetate (56a)

22 h (127 mg, 0.17 mmol), 56b (62 mg, 0.15 mmol) and 4 Å molecular sieve (300 mg) were dissolved in anhydrous DCE (20 mL) and the mixture was stirred at 25° C. for 1 h before addition of AgOTf (46 mg, 0.18 mmol) and NIS (41 mg, 0.18 mmol). The resulting mixture was firstly stirred at 0° C. for 2 h and further stirred at 25° C. for 16 h. After complete reaction, the mixture was filtered though kieselguhr and the filtrate was washed with saturated aqueous Na2S2O3 (20 mL) and brine (20 mL*2). The organic layer was dried over anhydrous Na2SO4, filtered and concentrated under reduced pressure to afford a brown oil as the crude product which was purified by flash column DCM/MeOH=100/0 to 85/15) to afford 56c (32 mg, 21.1% yield) as a white solid.

MS (ESI) m/z: 788.6 [M+Na]+

### Step 2

### (2R,3S,4S,5R,6R)-2-(acetoxymethyl)-6-(((11S,19R)-11-benzyl-19-((benzyloxy)carbonyl)-1-(9H-fluoren-9-yl)-3,6,9,12,15-pentaoxo-2,18-dioxa-4,7,10,13,16-pentaazaicosan-20-yl)oxy)tetrahydro-2H-pyran-3,4,5-triyl triacetate (56c)

22 h (127 mg, 0.17 mmol), 56b (62 mg, 0.15 mmol) and 4 Å molecular sieve (300 mg) were dissolved in anhydrous DCE (20 mL) and the mixture was stirred at 25° C. for 1 h before addition of AgOTf (46 mg, 0.18 mmol) and NIS (41 mg, 0.18 mmol). The resulting mixture was firstly stirred at 0° C. for 2 h and further stirred at 25° C. for 16 h. After complete reaction, the mixture was filtered though kieselguhr and the filtrate was washed with saturated aqueous Na2S203 (20 mL) and brine (20 mL*2). The organic layer was dried over anhydrous Na2SO4, filtered and concentrated under reduced pressure to afford a brown oil as the crude product which was purified by flash column DCM/MeOH=100/0 to 85/15) to afford 56c (32 mg, 21.1% yield) as a white solid.

MS (ESI) m/z: 1118.7 [M+Na]+

### Step 3

### (11S,19R)-11-benzyl-1-(9H-fluoren-9-yl)-3,6,9,12,15-pentaoxo-19-((((2R,3R,4S,5S,6R)-3,4,5-triacetoxy-6-(acetoxymethyl)tetrahydro-2H-pyran-2-yl)oxy)methyl)-2,18-dioxa-4,7,10,13,16-pentaazaicosan-20-oic acid (56d)

56d (30 mg, crude) was synthesized according to synthetic procedure of step 7 of example 22.

MS (ESI) m/z: 1028.8 [M+Na]+

### Step 4

### (2R,3S,4S,5R,6R)-2-(acetoxymethyl)-6-(((11S,19R)-11-benzyl-19-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)-1-(9H-fluoren-9-yl)-3,6,9,12,15-pentaoxo-2,18-dioxa-4,7,10,13,16-pentaazaicosan-20-yl)oxy)tetrahydro-2H-pyran-3,4,5-triyl triacetate (56f)

56f (33 mg, 77.7% yield) was synthesized according to synthetic procedure of step 8 of example 22.

MS (ESI) m/z: 1446.8 [M+Na]+

### Step 5

### (R)-2-(((S)-13-amino-7-benzyl-3,6,9,12-tetraoxo-2,5,8,11-tetraazatridecyl)oxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-(((2R,3R,4S,5R,6R)-3,4,5-trihydroxy-6-(hydroxymethyl)tetrahydro-2H-pyran-2-yl)oxy)propanamide (56 g)

56f (30 mg, 0.021 mmol) was dissolved in anhydrous MeOH (7 mL) followed by the addition of aqueous MeONa (1 M in MeOH, 0.011 mmol). The resulting mixture was stirred at 25° C. for 3 h. After complete reaction, the reaction mixture was neutralized with cation-exchange resin and purified by prep-HPLC (Method: column: XBridge Prep C18 OBD Sum 19*250 mm; Mobile phase: A—water (0.1% formic acid): B—acetonitrile; Flow rate: 20 mL/min) to afford 56 g (9 mg, 41.3% yield) as a white solid.

MS (ESI) m/z: 1033.7 [M+H]+

### Step 6

### (R)-2-(((S)-7-benzyl-20-(2,5-dioxo-2,5-dihydro-1H-pyrrol-1-yl)-3,6,9,12,15,19-hexaoxo-2,5,8,11,14,18-hexaazaicosyl)oxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-(((2R,3R,4S,5R,6R)-3,4,5-trihydroxy-6-(hydroxymethyl)tetrahydro-2H-pyran-2-yl)oxy)propanamide (56)

56 g (8 mg, 0.0077 mmol) was dissolved DMF (0.5 mL) followed by the addition of 56 h (13 mg, 0.039 mmol). The mixture was stirred at 25° C. for 15 min and purified by prep-HPLC (Method: column: XBridge Prep C18 OBD Sum 19*250 mm; Mobile phase: A—water (0.1% formic acid): B—acetonitrile; Flow rate: 20 mL/min) to afford 56 (6.5 mg, 67.6% yield) as a white solid.

MS (ESI) m/z: 1263.8 [M+Na]+

### Example 57

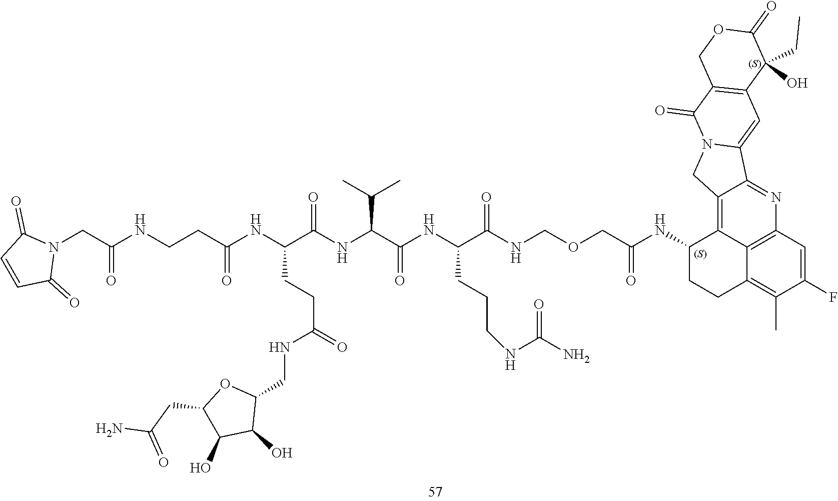

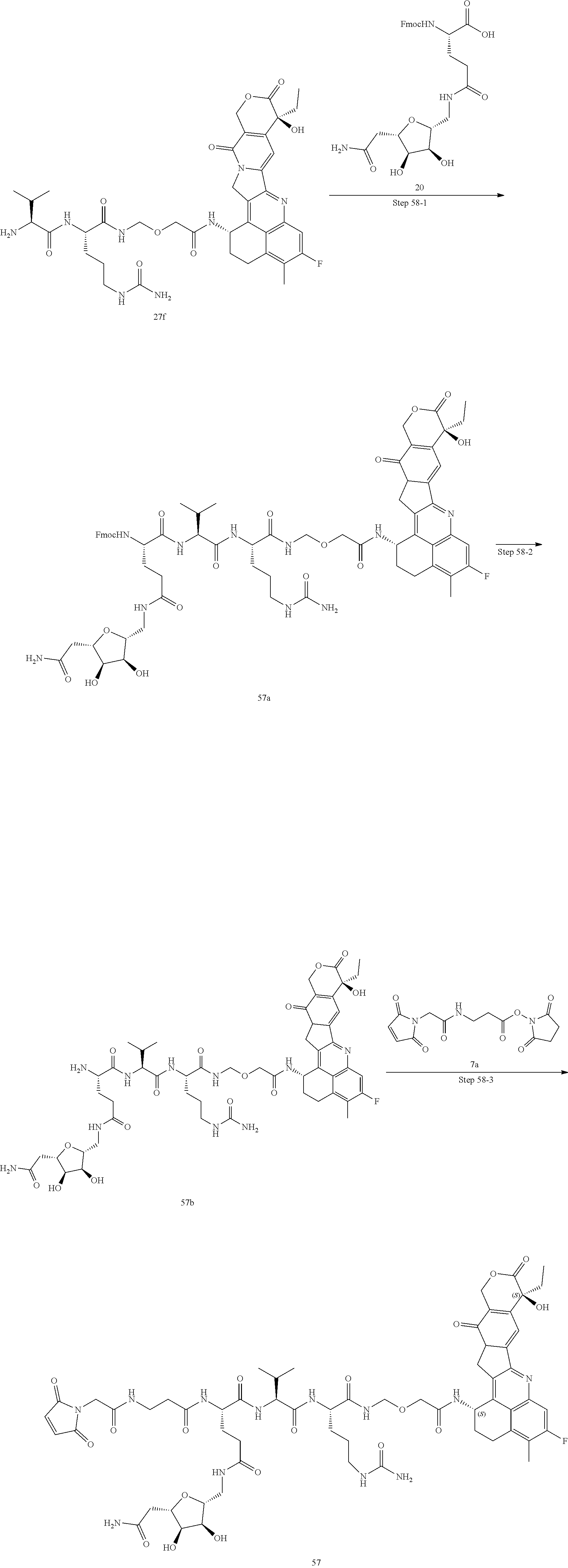

### Step 1

### (9H-fluoren-9-yl)methyl ((6S,9S,12S)-1-((2R,3 S,4R,5S)-5-(2-amino-2-oxoethyl)-3,4-dihydroxytetrahydrofuran-2-yl)-18-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-9-isopropyl-3,7,10,13,18-pentaoxo-12-(3-ureidopropyl)-16-oxa-2,8,11,14-tetraazaoctadecan-6-yl)carbamate (57a)

Compound 57a (64.0 mg, 44.400 yield) was synthesized according to synthetic procedure of step 1 of example 38.

MS (ESI) m/z: 1325.0 [M+Na]+

### Step 2

### (S)-2-amino-N5-(((2R,3S,4R,5S)-5-(2-amino-2-oxoethyl)-3,4-dihydroxytetrahydrofuran-2-yl)methyl)-N1-((S)-1-(((S)-1-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxo-5-ureidopentan-2-yl)amino)-3-methyl-1-oxobutan-2-yl)pentanediamide (57b)

Compound 57b (60.0 mg, crude) was synthesized according to synthetic procedure of step 3 of example 42.

MS (ESI) m/z: 1080.8 [M+H]+

### Step 3

### (S)—N5-(((2R,3S,4R,5S)-5-(2-amino-2-oxoethyl)-3,4-dihydroxytetrahydrofuran-2-yl)methyl)-2-(3-(2-(2,5-dioxo-2,5-dihydro-1H-pyrrol-1-yl)acetamido)propanamido)-N1-((S)-1-(((S)-1-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxo-5-ureidopentan-2-yl)amino)-3-methyl-1-oxobutan-2-yl)pentanediamide (50)

Compound 57 (16.1 mg, 45.0% yield) was synthesized according to synthetic procedure of step 8 of example 2.

MS (ESI) m/z: 1289.4 [M+H]+

### Example 58

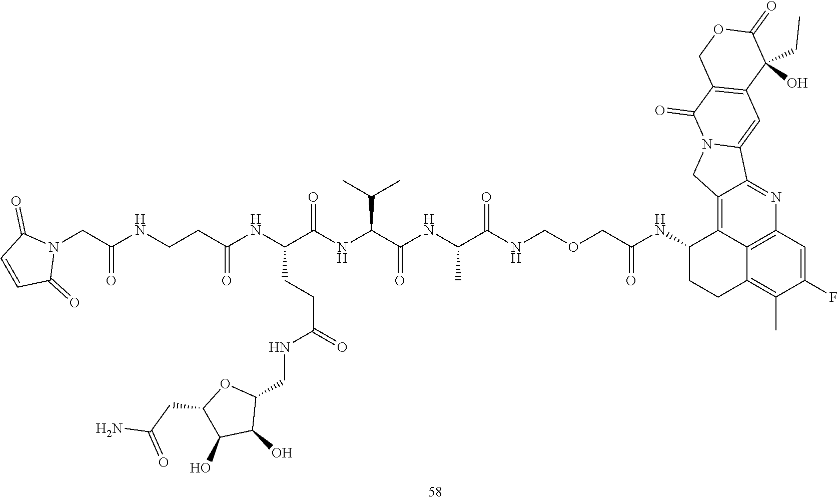

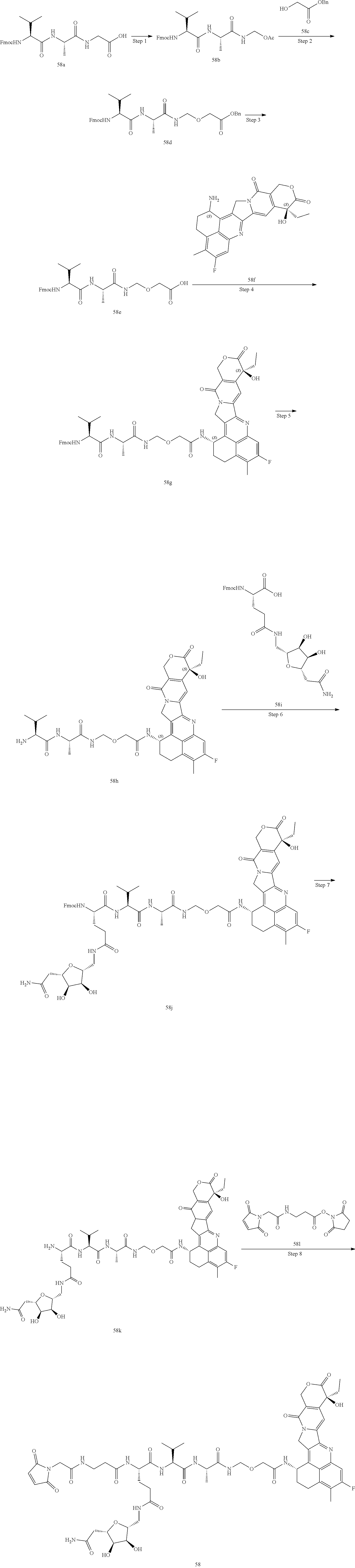

### Step 1

### (5S,8S)-1-(9H-fluoren-9-yl)-5-isopropyl-8-methyl-3,6,9-trioxo-2-oxa-4,7,10-triazaundecan-11-yl acetate (58b)

58b (850 mg, 82.5% yield) was synthesized according to the synthetic procedure of step 5 of example 22g.

### Step 2

### Benzyl (5S,8S)-1-(9H-fluoren-9-yl)-5-isopropyl-8-methyl-3,6,9-trioxo-2,12-dioxa-4,7,10-triazatetradecan-14-oate (58d)

58d (450 mg, 92% yield) was synthesized according to the synthetic procedure of step 6 of example 22h.

MS (ESI) m/z: 610.5 [M+Na]+.

### Step 3

### (5S,8S)-1-(9H-fluoren-9-yl)-5-isopropyl-8-methyl-3,6,9-trioxo-2,12-dioxa-4,7,10-triazatetradecan-14-oic acid (58e)

58e (215 mg, theoretical yield) was synthesized according to the synthetic procedure of step 4 of example 34f.

MS (ESI) m/z: 521.5 [M+Na]+.

### Step 4

### (9H-fluoren-9-yl)methyl ((S)-1-(((S)-1-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)amino)-3-methyl-1-oxobutan-2-yl)carbamate (58 g)

58 g (72 mg, theoretical yield) was synthesized according to the synthetic procedure of step 8 of example 22k.

MS (ESI) m/z: 937.7 [M+Na]+.

### Step 5

### (S)-2-amino-N—((S)-1-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)-3-methylbutanamide (58 h)

58 h (54.5 mg, theoretical yield) was synthesized according to the synthetic procedure of step 8 of example 34k.

MS (ESI) m/z: 693.6 [M+H]+.

### Step 6

### (9H-fluoren-9-yl)methyl ((6S,9S,12S)-1-((2R,3S,4R,5S)-5-(2-amino-2-oxoethyl)-3,4-dihydroxytetrahydrofuran-2-yl)-18-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-9-isopropyl-12-methyl-3,7,10,13,18-pentaoxo-16-oxa-2,8,11,14-tetraazaoctadecan-6-yl)carbamate (58j)

58j (42 mg, 43.9% yield) was synthesized according to the synthetic procedure of step 7 of example 34j.

MS (ESI) m/z: 1239.0 [M+Na]+.

### Step 7

### (S)-2-amino-N5-(((2R,3S,4R,5S)-5-(2-amino-2-oxoethyl)-3,4-dihydroxytetrahydrofuran-2-yl)methyl)-N1-((S)-1-(((S)-1-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)amino)-3-methyl-1-oxobutan-2-yl)pentanediamide (58k)

58k (34.3 mg, theoretical yield) was synthesized according to the synthetic procedure of step 8 of example 34k.

MS (ESI) m/z: 994.9 [M+H]+.

### Step 8

### (S)—N5-(((2R,3S,4R,5S)-5-(2-amino-2-oxoethyl)-3,4-dihydroxytetrahydrofuran-2-yl)methyl)-2-(3-(2-(2,5-dioxo-2,5-dihydro-1H-pyrrol-1-yl)acetamido)propanamido)-N1-((S)-1-(((S)-1-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)amino)-3-methyl-1-oxobutan-2-yl)pentanediamide (58)

58 (22.1 mg, 53.1% yield) was synthesized according to the synthetic procedure of step 4 of example 39.

MS (ESI) m/z: 1225.2 [M+Na]+.

### Example 59

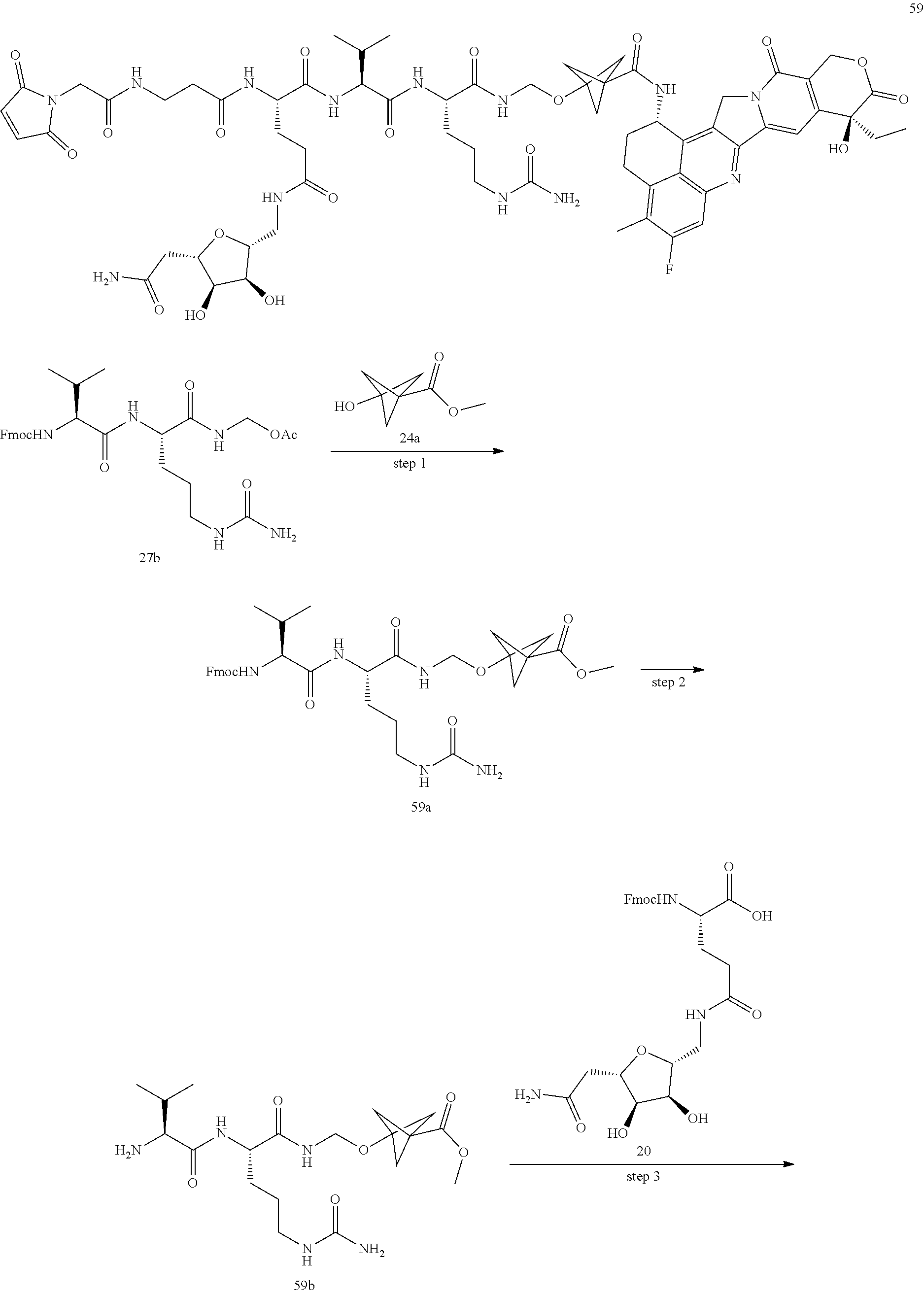

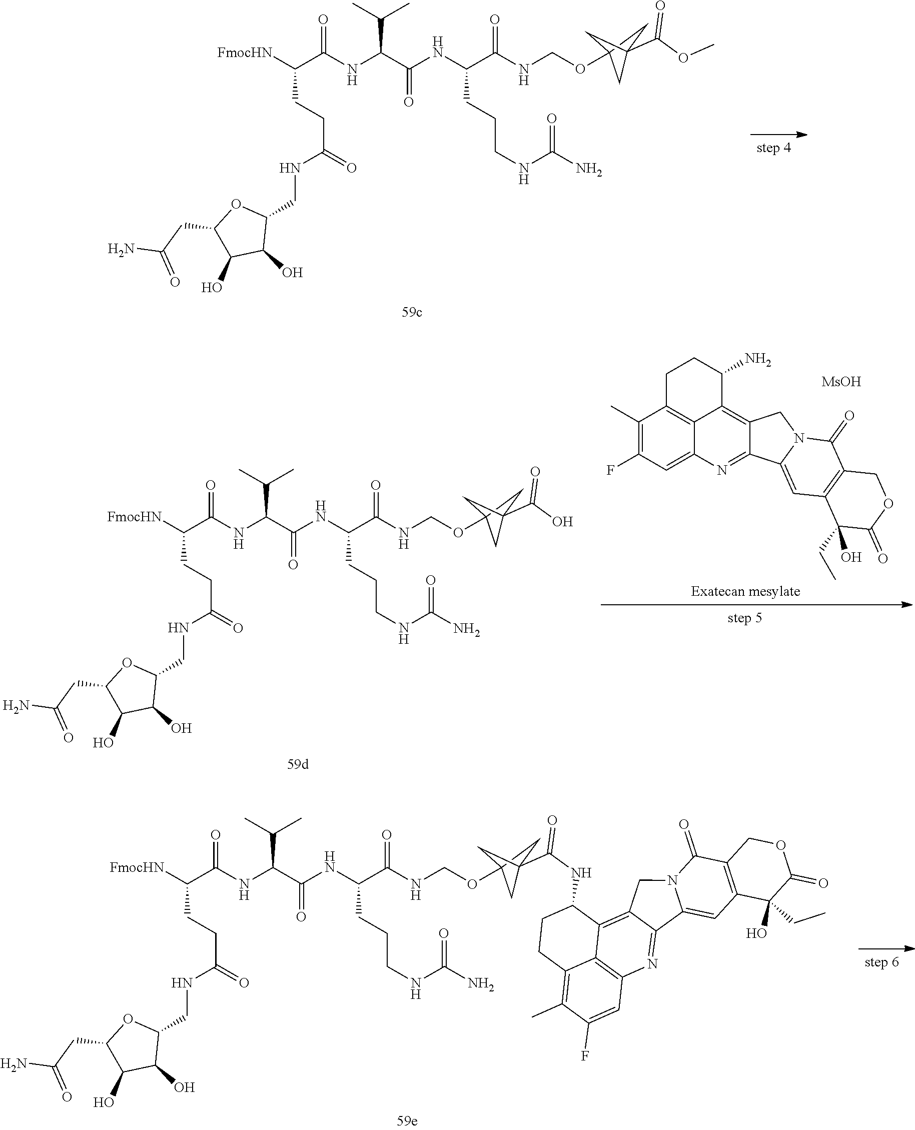

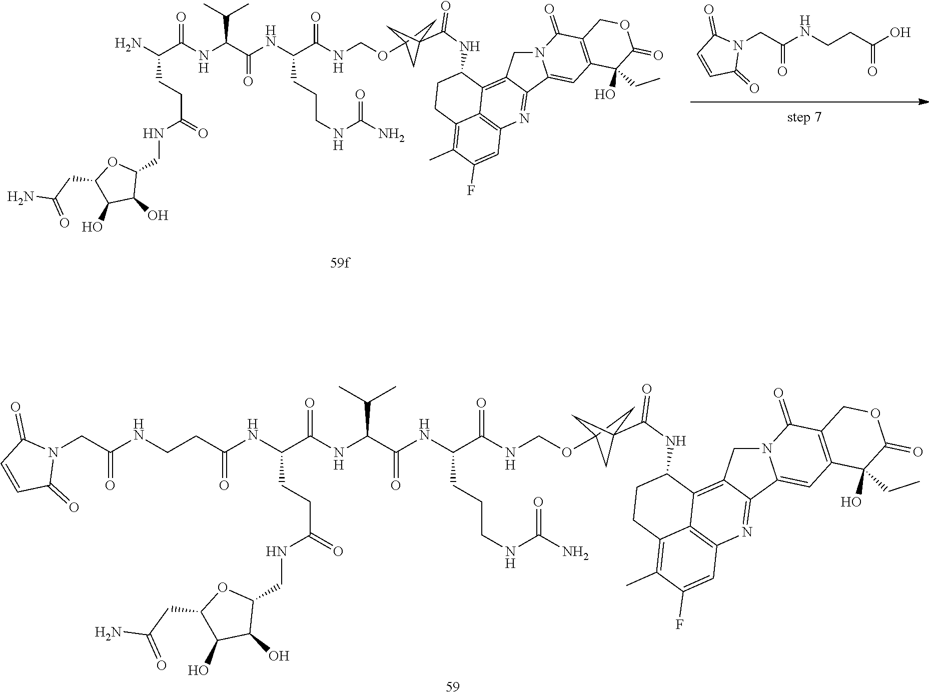

### Step 1

### Methyl 3-(((5S,8S)-1-(9H-fluoren-9-yl)-5-isopropyl-3,6,9-trioxo-8-(3-ureidopropyl)-2-oxa-4,7,10-triazaundecan-11-yl)oxy)bicyclo[1.1.1]pentane-11-carboxylate (59a)

To a mixture of 27b (300 mg, 0.53 mmol), 24a (188 mg, 1.32 mmol) and dried 4 Å molecular sieves (1.2 g) in dry THF (6 mL) was added scandium trifluoromethanesulfonate (392 mg, 0.79 mmol). The resulting mixture was stirred at r.t. overnight. The solution was filtered through Celite and quenched with saturated NaHCO3 aq. (15 mL). The aqueous phase was extracted with ethyl acetate (20 mL*3). The combined organic layer was dried over anhydrous sodium sulfate. After filtration and evaporation, the residue was purified by flash column (eluent: DCM/MeOH, from 100/0 to 10/1 (v/v)) to afford 59a (250 mg, 72.8% yield).

MS (ESI) m/z: 672.6 [M+Na]⁺

### Step 2

### Methyl 3-(((S)-2-((S)-2-amino-3-methylbutanamido)-5ureidopentanamido)methoxy)bicyclo[1.1.1]pentane-1-carboxylate (59b)

To the solution of 59a (120 mg, 0.18 mmol) in DMF (3 mL) was added diethylamine (203 mg, 2.77 mmol). The reaction was stirred at r.t. for 1 h. The reaction solution was extracted with Pet. ether (3 mL*3). The aqueous solution was concentrated to give the title compound 59b (77 mg, crude), which was used directly without further purification.

MS (ESI) m/z: 450.2 [M+Na]+

### Step 3

### Methyl 3-(((5S,8S,11S)-5-(3-((((2R,3S,4R,5S)-5-(2-amino-2-oxoethyl)-3,4-dihydroxytetrahydrofuran-2-yl)methyl)amino)-3-oxopropyl)-1-(9H-fluoren-9-yl)-8-isopropyl-3,6,9,12-tetraoxo-11-(3-ureidopropyl)-2-oxa-4,7,10,13-tetraazatetradecan-14-yl)oxy)bicyclo[1.1.1]pentane-1-carboxylate (59c)

To the solution of 20 (107 mg, 0.20 mmol) and TSTU (60 mg, 0.20 mmol) in dry DMF (3 mL) was added DIEA (90 μL, 0.54 mmol). The resulting mixture was stirred at r.t. for 10 min. 59b (77 mg, 0.18 mmol) was added. The reaction was stirred at r.t. for 1 h. The solution was filtered and purified by prep-HPLC (0.1% FA in water/MeCN), lyophilized to give the title compound 59c (50 mg, 29.2% yield) as a white solid.

MS (ESI) m/z: 973.8 [M+Na]+

### Step 4

### 3-(((5S,8S,11S)-5-(3-((((2R,3S,4R,5S)-5-(2-amino-2-oxoethyl)-3,4-dihydroxytetrahydrofuran-2-yl)methyl)amino)-3-oxopropyl)-1-(9H-fluoren-9-yl)-8-isopropyl-3,6,9,12-tetraoxo-11-(3-ureidopropyl)-2-oxa-4,7,10,13-tetraazatetradecan-14-yl)oxy)bicyclo[1.1.1]pentane-1-carboxylic acid (59d)

To the solution of 59c (50 mg, 0.05 mmol) in THE (1 mL) and water (1 mL) was added lithium hydroxide (1N in water)(0.75 mL, 0.75 mmol). The reaction was stirred at 0° C. for 5 h. The aqueous solution was concentrated to give the title compound 59d (46 mg, crude), which was used directly without further purification.

MS (ESI) m/z: 959.7 [M+Na]+

### Step 5

### (9H-fluoren-9-yl)methyl ((6S,9S,12S)-1-amino-17-((2R,3S,4R,5S)-5-(2-amino-2-oxoethyl)-3,4-dihydroxytetrahydrofuran-2-yl)-6-((((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)bicyclo[1.1.1]pentan-1-yl)oxy)methyl)carbamoyl)-9-isopropyl-1,8,11,15-tetraoxo-2,7,10,16-tetraazaheptadecan-12-yl)carbamate (59e)

To the solution of 59d (46 mg, 0.05 mmol), exatecan mesylate (29 mg, 0.05 mmol) and HATU (19.6 mg, 0.05 mmol) in dry DMF (2 mL) was added DIEA (30 μL, 0.15 mmol). The reaction was stirred at r.t. for 1 min. The solution was filtered and purified by prep-HPLC (0.1% FA in water/MeCN), lyophilized to give the title compound 59e (40 mg, 60.2% yield) as a white solid.

MS (ESI) m/z: 1377.9 [M+Na]+

### Step 6

### (S)-2-amino-N5-(((2R,3S,4R,5S)-5-(2-amino-2-oxoethyl)-3,4-dihydroxytetrahydrofuran-2-yl)methyl)-N1-((S)-1-(((S)-1-((((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)bicyclo[1.1.1]pentan-1-yl)oxy)methyl)amino)-1-oxo-5-ureidopentan-2-yl)amino)-3-methyl-1-oxobutan-2-yl)pentanediamide (59f)

To the solution of 59e (40 mg, 0.03 mmol) in DMF (2 mL) was added diethylamine (202.8 mg, 2.77 mmol). The reaction was stirred at r.t. for 1 h. The reaction solution was extracted with Pet. ether (3 mL*3). The aqueous solution was concentrated to give the title compound 59f (31 mg, crude), which was used directly without further purification.

MS (ESI) m/z: 1132.8 [M+H]+

### Step 7

### (S)—N5-(((2R,3S,4R,5S)-5-(2-amino-2-oxoethyl)-3,4-dihydroxytetrahydrofuran-2-yl)methyl)-2-(3-(2-(2,5-dioxo-2,5-dihydro-1H-pyrrol-1-yl)acetamido)propanamido)-N1-((S)-1-(((S)-1-((((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)bicyclo[1.1.1]pentan-1-yl)oxy)methyl)amino)-1-oxo-5-ureidopentan-2-yl)amino)-3-methyl-1-oxobutan-2-yl)pentanediamide (59)

To the solution of 59f (31 mg, 0.05 mmol), Exatecan mesylate (6.8 mg, 0.03 mmol) and HATU (10.94 mg, 0.03 mmol) in dry DMF (1 mL) was added DIEA (10 μL, 0.04 mmol). The reaction was stirred at r.t. for 15 min. The solution was filtered and purified by prep-HPLC (0.1% FA in water/MeCN), lyophilized to give the title compound 59 (12.6 mg, 34.3% yield) as a white solid.

MS (ESI) m/z: 1362.8 [M+Na]+

### Example 60

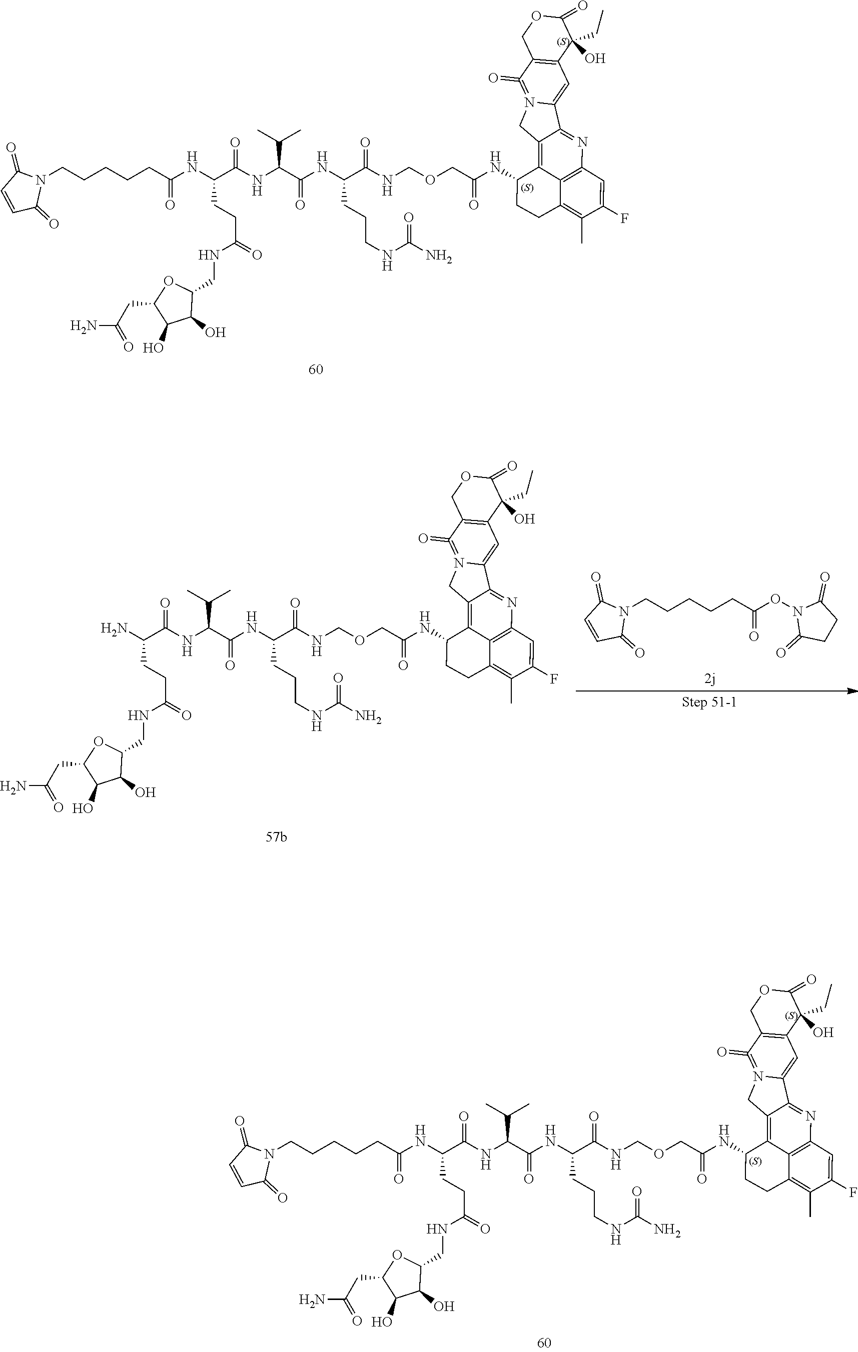

### Step 1

### (S)—N5-(((2R,3S,4R,5S)-5-(2-amino-2-oxoethyl)-3,4-dihydroxytetrahydrofuran-2-yl)methyl)-2-(6-(2,5-dioxo-2,5-dihydro-1H-pyrrol-1-yl)hexanamido)-N1-((S)-1-(((S)-1-(((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxo-5-ureidopentan-2-yl)amino)-3-methyl-1-oxobutan-2-yl)pentanediamide (67)

Compound 60 (18.0 mg, 33.9% yield) was synthesized according to synthetic procedure of step 8 of example 2.

MS (ESI) m/z: 1274.3 [M+H]+

### Example 61

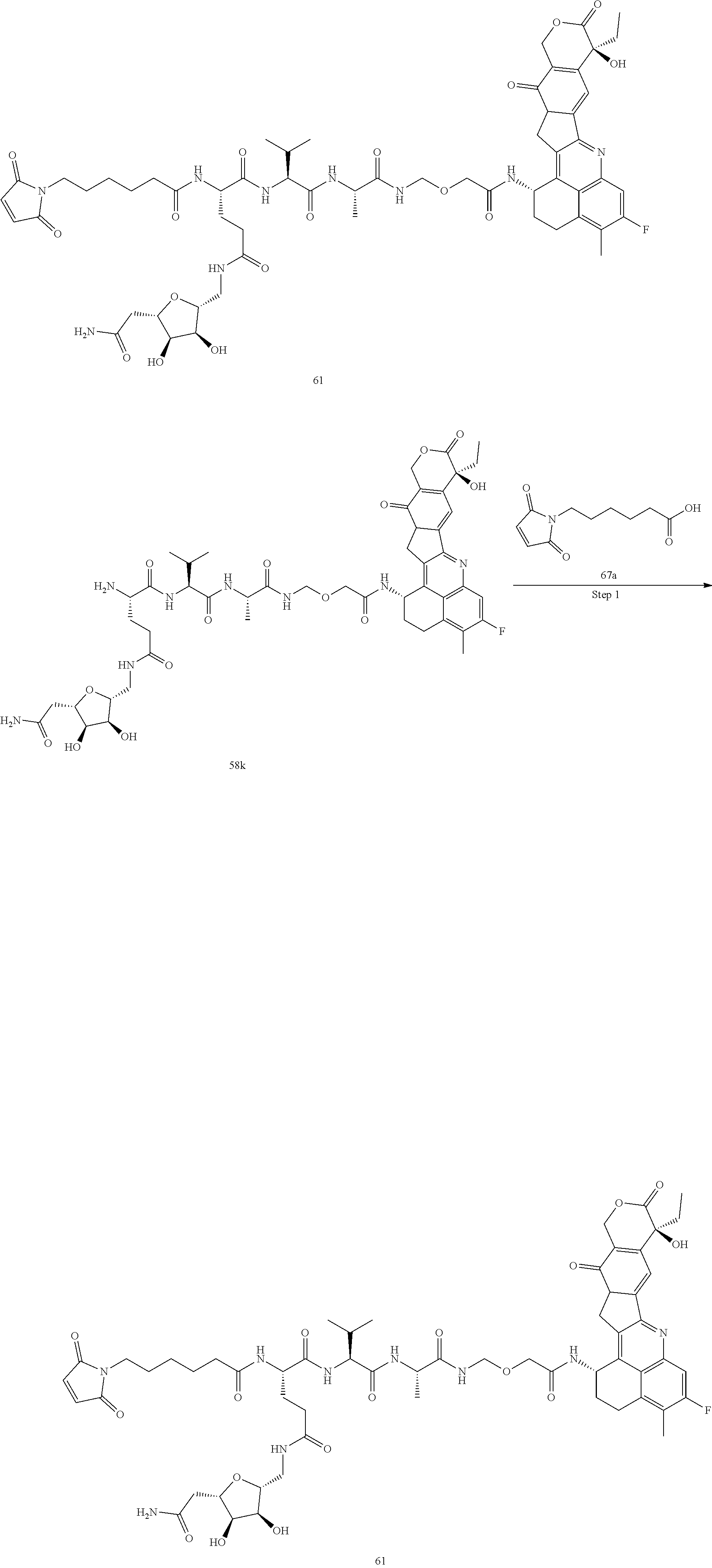

61 (56.4 mg, 79.1% yield) was synthesized according to the synthetic procedure of step 9 of example 34.

MS (ESI) m/z: 1209.9 [M+Na]⁺.

### Example 62

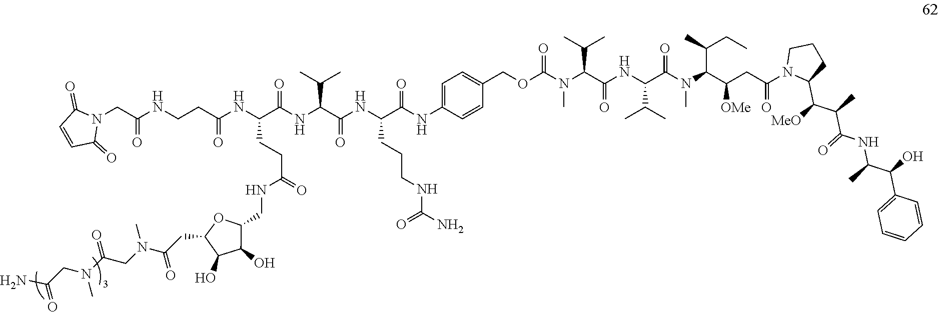

### Example 62

62 (18.8 mg, 45.1% yield) was synthesized according to the synthetic procedures of example 9.

MS(ESI) m/z: 1918.0 [M+H]⁺.

### Example 63

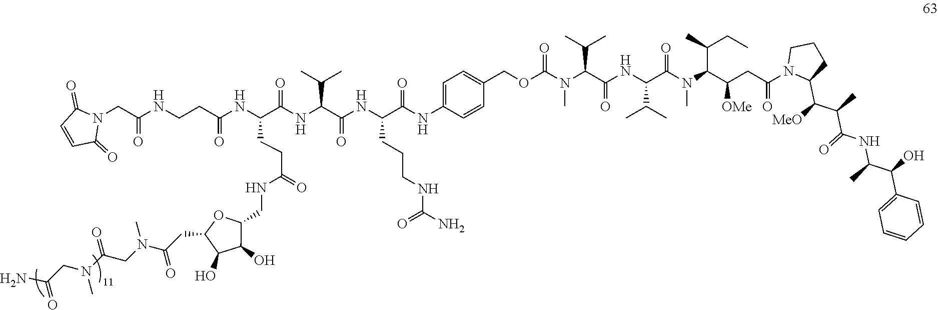

### Example 63

63 (24.3 mg, 58.1% yield) was synthesized according to the synthetic procedures of example 9.

MS(ESI) m/z: 2486.5 [M+H]⁺.

### Example 64

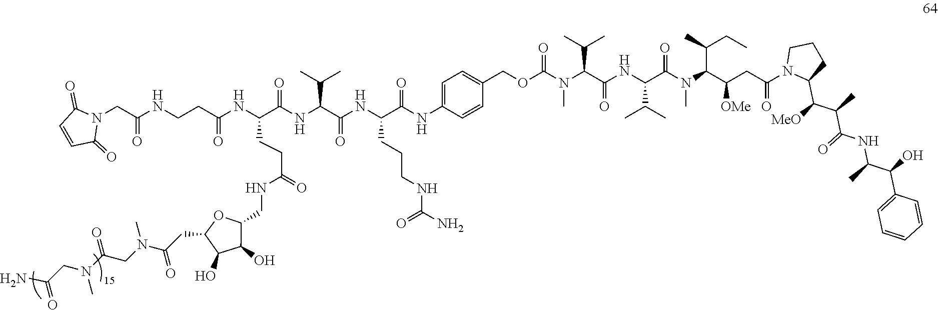

### Example 64

Compound 64 (9.8 mg, 35.2% yield) was synthesized according to the synthetic procedures of example 9. MS(ESI) m/z: 2771.1 [M+H]⁺.

### Example 65

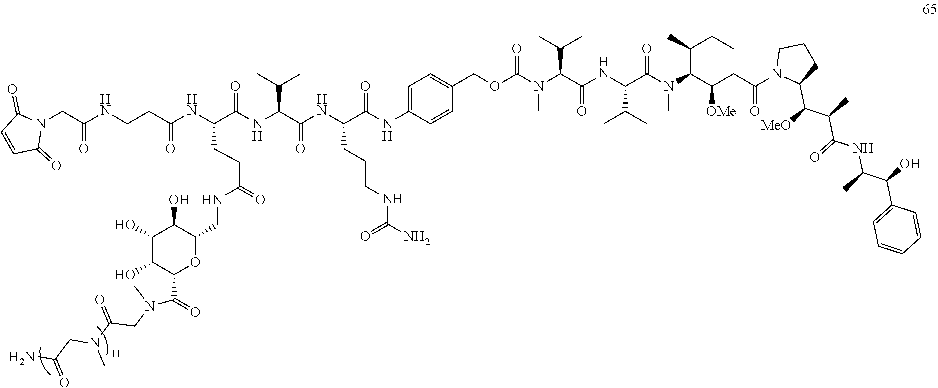

Compound 65 (22.8 mg, 41.8% yield) was synthesized according to the synthetic procedures of examples 9 and 50. MS (ESI) m/z: 2502.9 [M+H]⁺.

### Example 66

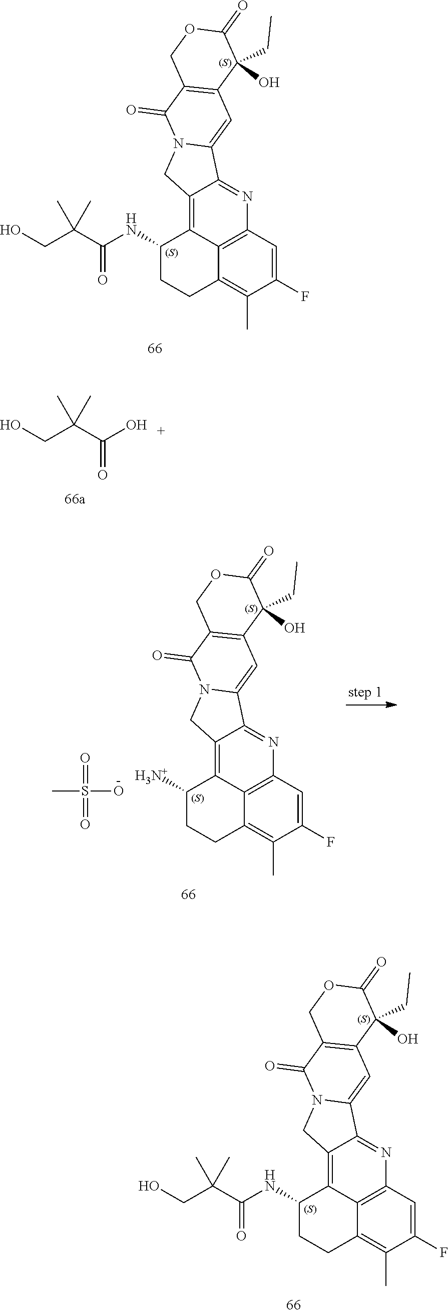

### Step 1

### N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxy-2,2-dimethylpropanamide (66)

To a mixture of 66a (5 mg, 0.042 mmol) and HATU (16 mg, 0.042 mmol) in DMF (1 mL) were added DIPEA (21 μL, 16 mg, 0.13 mmol) and 66b (Purchased from ShangHai HaoYuan MedChemExpress CO. LTD, 23 mg, 0.043 mmol). The resulted brown mixture was stirred at r.t. for 2 h. On completion of the reaction, the mixture was purified by prep-HPLC (TFA) (Method: column XBridge Prep C18 OBD Sum 19*250 mm; Mobile phase: A—water (0.1% TFA): B—acetonitrile; Flow rate: 20 mL/min, the fraction was lyophilized to give 66 (15 mg, 65.5% yield) as a white powder.

¹H NMR (400 MHz, DMSO): δ 8.00 (d, J=8.4 Hz, 1H), 7.79 (d, J=11.2 Hz, 1H), 7.31 (s, 1H), 6.52 (s, 1H), 5.59-5.54 (m, 1H), 5.42 (s, 2H), 5.18 (q, J=19.2 Hz, 2H), 4.87 (t, J=5.2 Hz, 1H), 3.45 (dd, J=10.2, 4.8 Hz, 1H), 3.41-3.28 (m, 1H), 3.15 (t, J=5.6 Hz, 2H), 2.40 (s, 3H), 2.24-2.07 (m, 2H), 1.92-1.80 (m, 2H), 1.11 (d, J=7.6 Hz, 6H), 0.87 (t, J=7.2 Hz, 3H). MS (ESI) m/z: 536.4 [M+H]⁺

### Example 67

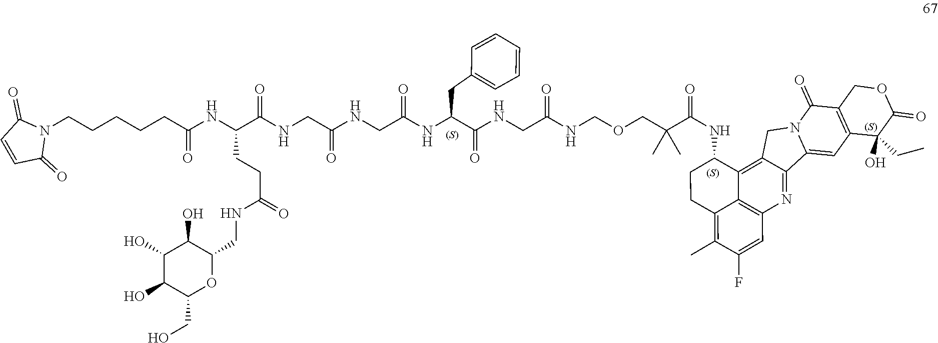

### Step 1

### Benzyl (S)-11-benzyl-1-(9H-fluoren-9-yl)-20,20-dimethyl-3,6,9,12,15-pentaoxo-2,18-dioxa-4,7,10,13,16-pentaazahenicosan-21-oate (67c)

To solution of 67a (250 mg, 0.40 mmol) and 67b (83 mg, 0.40 mmol) in THE (5 mL) was added 4 Å molecular sieve. The mixture was stirred at r.t. for 10 min then Sc(OTf)₃ (195 mg, 0.40 mmol) was added and reacted at r.t. for another 16 h. The suspension mixture was filtered through a pad of celite, and the cake was washed with THE (10 mL) then the filtrate was quenched by addition of Sat. NaHCO3 (10 mL), extracted with EtOAc (30 mL*2). After separation the combined organic layers were washed with brine (50 mL), dried over Na₂SO₄, filtered and the filtrate was concentrated under vacuum to give a residue. It was purified by silica gel column chromatography (A—DCM; B—MeOH, MeOH/DCM=0/100 to 95/5) to provide 67c (90 mg, 29.2% yield).

MS (ESI) m/z: 800.5 [M+Na]⁺

### Step 2

### (S)-11-benzyl-1-(9H-fluoren-9-yl)-20,20-dimethyl-3,6,9,12,15-pentaoxo-2,18-dioxa-4,7,10,13,16-pentaazahenicosan-21-oic acid (67d)

To a solution of 67c (80 mg, 0.10 mmol) in MeOH (3 mL) was added wet Pd/C (20 mg). The black suspension was purged with H2 balloon for three times then reacted at r.t. for 2 h under H2 balloon. After the reaction was completed, the black suspension was filtered off through a pad of celite and the cake wash with MeOH, the combined organic layers were concentrated under vacuum to provide 67d (61 mg, 84.8% yield).

MS (ESI) m/z: 710.4 [M+Na]⁺

### Step 3

### (9H-fluoren-9-yl)methyl ((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)carbamate (67f)

To a mixture of 67d (60 mg, 0.087 mmol) and HATU (33 mg, 0.087 mmol) in DMF (2 mL) was added DIPEA (43 μL, 34 mg, 0.26 mmol). The mixture was reacted at r.t. for 10 min. 67e (46 mg, 0.087 mmol) was added and reacted at the same temperature for another 1 hr. After the reaction was completed, the mixture was filtered and the filtrate was purified using prep-HPLC (Method; column: XBridge Prep C18 OBD 5 μm 19*250 mm; Mobile phase: A—water (0.1% formic acid): B—acetonitrile; Flow rate: 20 mL/min) to provide 67f (85 mg, 88.2% yield).

MS (ESI) m/z: 1105.5 [M+H]⁺

### Step 4

### 3-(((S)-13-amino-7-benzyl-3,6,9,12-tetraoxo-2,5,8,11-tetraazatridecyl)oxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2,2-dimethylpropanamide (67 g)

To a solution of 67f (85 mg, 0.062 mmol) in DMF (2 mL) was added Et₂N (64 μL, 46 mg, 0.62 mmol). The mixture was stirred at r.t. for 0.5 h. On completion of the reaction. The mixture was concentrated under vacuum to give 67 g (86 mg, crude) as a yellow solid.

MS (ESI) m/z: 883.5 [M+H]⁺

### Step 6

### (9H-fluoren-9-yl)methyl ((6S,15S)-15-benzyl-25-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-24,24-dimethyl-3,7,10,13,16,19,25-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazapentacosan-6-yl)carbamate (67i)

To a solution of 67 g (86 mg, crude) and 67 h (43 mg, 0.079 mmol) in DMF (1.5 mL) was added DIPEA (26 μL, 21 mg, 0.16 mmol). The mixture was stirred at r.t. for 1.5 h. On completion of the reaction. The mixture was purified by prep-HPLC (FA) (Method: column: XBridge Prep C18 OBD Sum 19*250 mm; Mobile phase: A—water (0.1% formic acid): B—acetonitrile; Flow rate: 20 mL/min, the fraction was lyophilized to give 67i (70 mg, 62.6% yield) as a white powder.

MS (ESI) m/z: 1410.7 [M+H]⁺

### Step 7

### (S)-2-amino-N1-((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)-N5-(((2S,3R,4R,5S,6R)-3,4,5-trihydroxy-6-(hydroxymethyl)tetrahydro-2H-pyran-2-yl)methyl)pentanediamide (67j)

To a solution of 67i (70 mg, 0.050 mmol) in DMF (1 mL) was added Et₂N (51 μL, 36 mg, 0.50 mmol). The mixture was stirred at r.t. for 0.5 h. On completion of the reaction. The mixture was concentrated under vacuum to give 67j (71 mg, crude) as a yellow solid.

MS (ESI) m/z: 1188.2 [M+H]⁺

### Step 8

### (S)—N¹—((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)-2-(6-(2,5-dioxo-2,5-dihydro-1H-pyrrol-1-yl)hexanamido)-N⁵-(((2S,3R,4R,5S,6R)-3,4,5-trihydroxy-6-(hydroxymethyl)tetrahydro-2H-pyran-2-yl)methyl)pentanediamide (67)

To a solution of 67k (19 mg) in DMF (3 mL) were added HATU (34 mg, 0.088 mmol) and DIPEA (10 μL, 7.6 mg, 0.059 mmol). The resulted yellow solution was stirred at r.t. for 5 min then 67j (71 mg, crude) was added. The mixture was stirred at r.t. for 60 min. On completion of the reaction. The mixture was purified by prep-HPLC (FA) (Method: column: XBridge Prep C18 OBD Sum 19*250 mm; Mobile phase: A—water (0.1% formic acid): B—acetonitrile; Flow rate: 20 mL/min, the fraction was lyophilized to give 67 (32 mg, 26.3% yield) as a white powder.

MS (ESI) m/z: 1381.1 [M+H]⁺

### Example 68 & 69

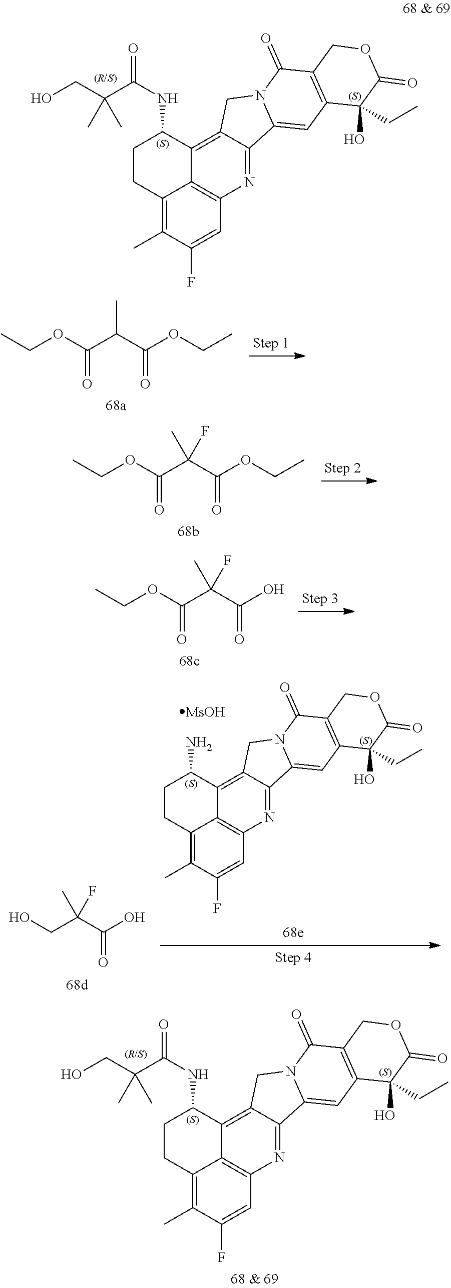

### Step 1

### Diethyl 2-fluoro-2-methylmalonate (68b)

A solution of compound 68a (10.0 g, 57.4 mmol) in THE (200 mL) was cooled to 0° C. 60% of NaH in oil (3.21 g, 80.37 mmol) was added into the mixture portionwise, stirred at 0° C. for 30 min. Then N-fluoro-N-(phenylsulfonyl)benzenesulfonamide (NSFI, 19.91 g, 63.2 mmol) was added into the mixture portionwise at 0° C. Then warmed up to r.t. and stirred for 16 h. After reaction completed, the suspension was filtered and the filtrated was concentrated. PE (100 mL) was added into the residue, the precipitate was filtered and the filtrated was concentrated to give compound 68b (12.5 g, crude) as a light-yellow oil.

¹H NMR (400 MHz, CDCl₃) δ 4.30 (q, J=7.2 Hz, 4H), 1.79 (d, J=22.0 Hz, 3H), 1.31 (t, J=7.2 Hz, 6H); ¹⁹F NMR (376 MHz, CDCl₃) δ −157.50.

### Step 2

### 3-ethoxy-2-fluoro-2-methyl-3-oxopropanoic acid (68c)

To a solution of compound 68b (1.0 g, 5.2 mmol) in EtOH (5 mL) was added KOH solution (321 mg) in H2O (50 uL) and EtOH (2 mL) dropwise at 0° C. The mixture was stirred at r.t. for 2 h. The mixture was diluted with 20 (mL), washed with DCM (20 mL*3). The aqueous solution was adjusted to pH=3 by 1 N HCl. The extracted by EA (50 mL*3). The organic layer was dried combined and dried over anhydrous Na₂SO₄, filtered, and concentrated to give compound 68c (470 mg, 55.0% yield) as colorless oil.

¹H NMR (400 MHz, CDCl₃) δ 8.31 (br s, 1H), 4.32 (q, J=7.2 Hz, 2H), 1.83 (d, J=22.0 Hz, 3H), 1.33 (t, J=7.2 Hz, 3H); ¹⁹F NMR (376 MHz, CDCl₃) δ −157.59.

### Step 3

### 2-fluoro-3-hydroxy-2-methylpropanoic acid (68d)

To a solution of compound 68c (200 mg, 1.22 mmol) in isopropanol (4 mL) was added 2 M LiBH4 (1.22 mL, 2.44 mmol) at 0° C. The mixture was stirred at r.t. for 2 h. The mixture was quenched by 2 N HCl (1.22 mL) dropwise at 0° C. The diluted with H₂O (10 mL), extracted with EA (50 mL*3). The organic layer was combined and dried over anhydrous Na₂SO₄, filtered and concentrated to give compound 68d (92 mg, 61.7% yield) as colorless oi.

¹H NMR (400 MHz, CDCl₃) δ 4.01-3.81 (m, 2H), 1.58 (d, J=21.2 Hz, 3H); ¹⁹F NMR (376 MHz, CDCl₃) δ −163.98.

### Step 4

### N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-fluoro-3-hydroxy-2-methylpropanamide (68 & 69)

To a solution of compound 68d (23 mg, 0.19 mmol) in DMF (2 mL) were added compound 68e (50 mg, 0.094 mmol), HATU (54 mg, 141 mmol) and DIEA (36 mg, 0.28 mmol). The mixture was stirred at r.t. for 1 h. The mixture was purified by prep-HPLC (FA) (Method: column: XBridge Prep C18 OBD Sum 19*250 mm; Mobile phase: A—water (0.1% formic acid): B—acetonitrile; Flow rate: 20 mL/min, the fraction was lyophilized to give two isomers:

Isomer 1: white solid, 68 (11 mg, 21.9% yield). UPLC-MS, RT=3.52 min.

¹H NMR (400 MHz, DMSO-d₆) δ 9.06 (dd, J=9.0, 2.8 Hz, 1H), 8.00 (d, J=10.9 Hz, 1H), 7.54 (s, 1H), 6.75 (s, 1H), 5.82 (d, J=8.0 Hz, 1H), 5.65 (s, 2H), 5.43 (dt, J=77.8, 12.4 Hz, 3H), 4.17-3.91 (m, 1H), 3.83 (ddd, J=18.0, 12.4, 5.6 Hz, 1H), 3.40-3.27 (m, 1H), 2.62 (s, 3H), 2.50-2.34 (m, 2H), 2.22-1.98 (m, 2H), 1.81 (d, J=21.4 Hz, 3H), 1.11 (t, J=7.2 Hz, 3H); MS (ESI) m/z: 540.3 [M+H]⁺.

Isomer 2: white solid, 69 (8.4 mg, 16.6% yield). UPLC-MS, RT=3.86 min.

¹H NMR (400 MHz, DMSO-d₆) δ 8.72 (dd, J=8.4, 2.4 Hz, 1H), 7.78 (d, J=11.2 Hz, 1H), 7.31 (s, 1H), 6.52 (s, 1H), 5.58 (d, J=8.0 Hz, 1H), 5.42 (s, 2H), 5.32-5.05 (m, 3H), 3.83 (dd, J=26.8, 12.0 Hz, 1H), 3.61 (dd, J=21.6, 12.0 Hz, 1H), 3.22-3.07 (m, 2H), 2.46-2.30 (m, 3H), 2.28-2.05 (m, 2H), 2.02-1.74 (m, 2H), 1.45 (d, J=21.4 Hz, 3H), 0.87 (t, J=7.2 Hz, 3H); MS (ESI) m/z: 540.3 [M+H]⁺.

### Example 70 & 71

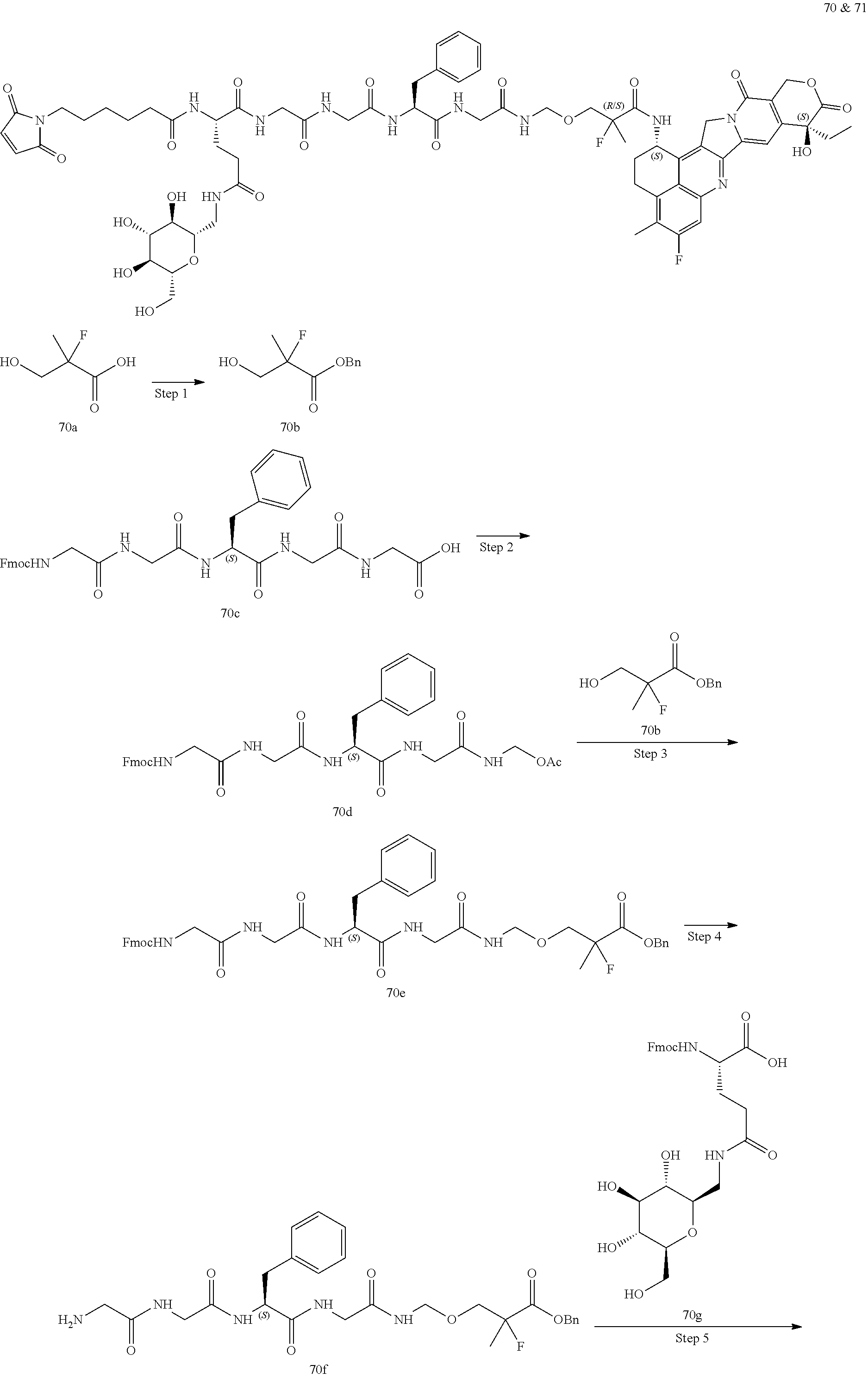

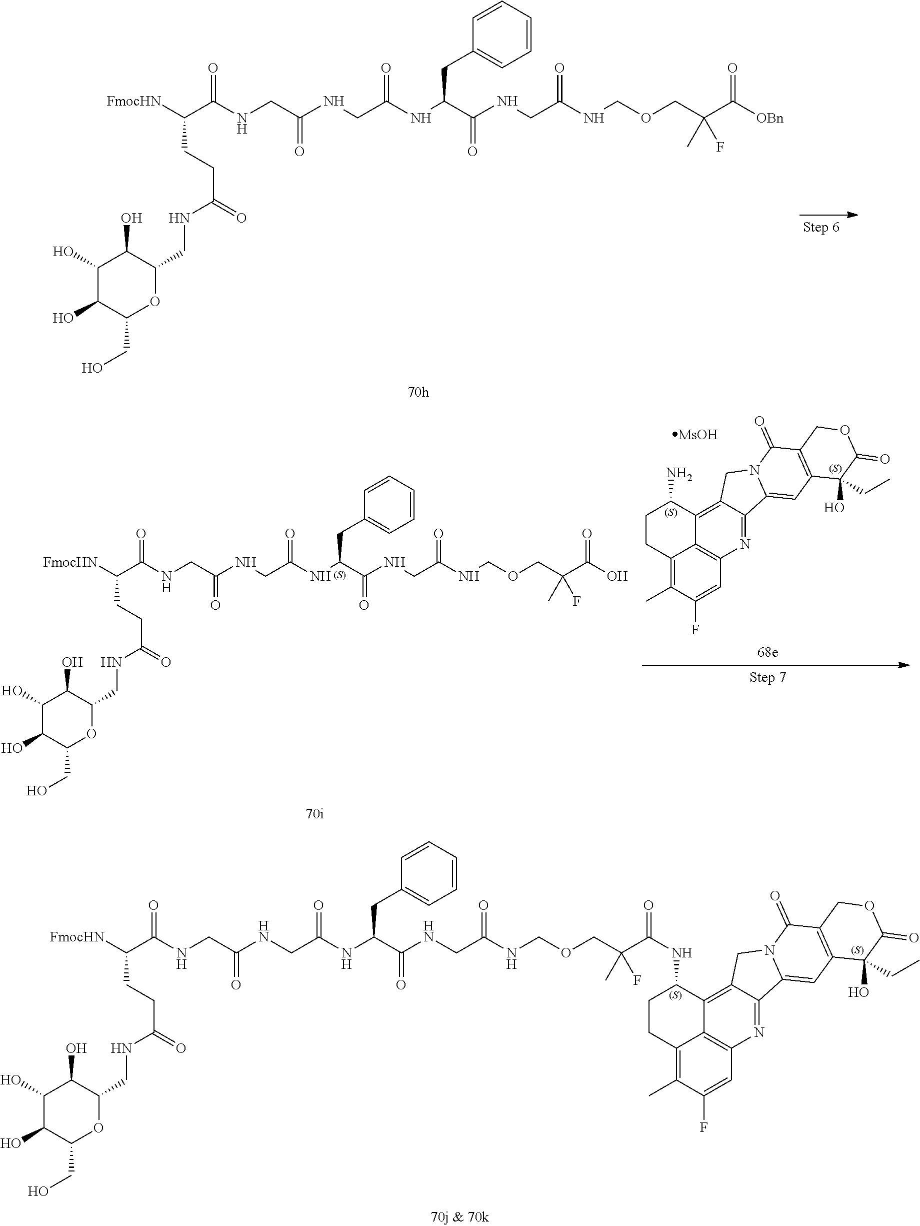

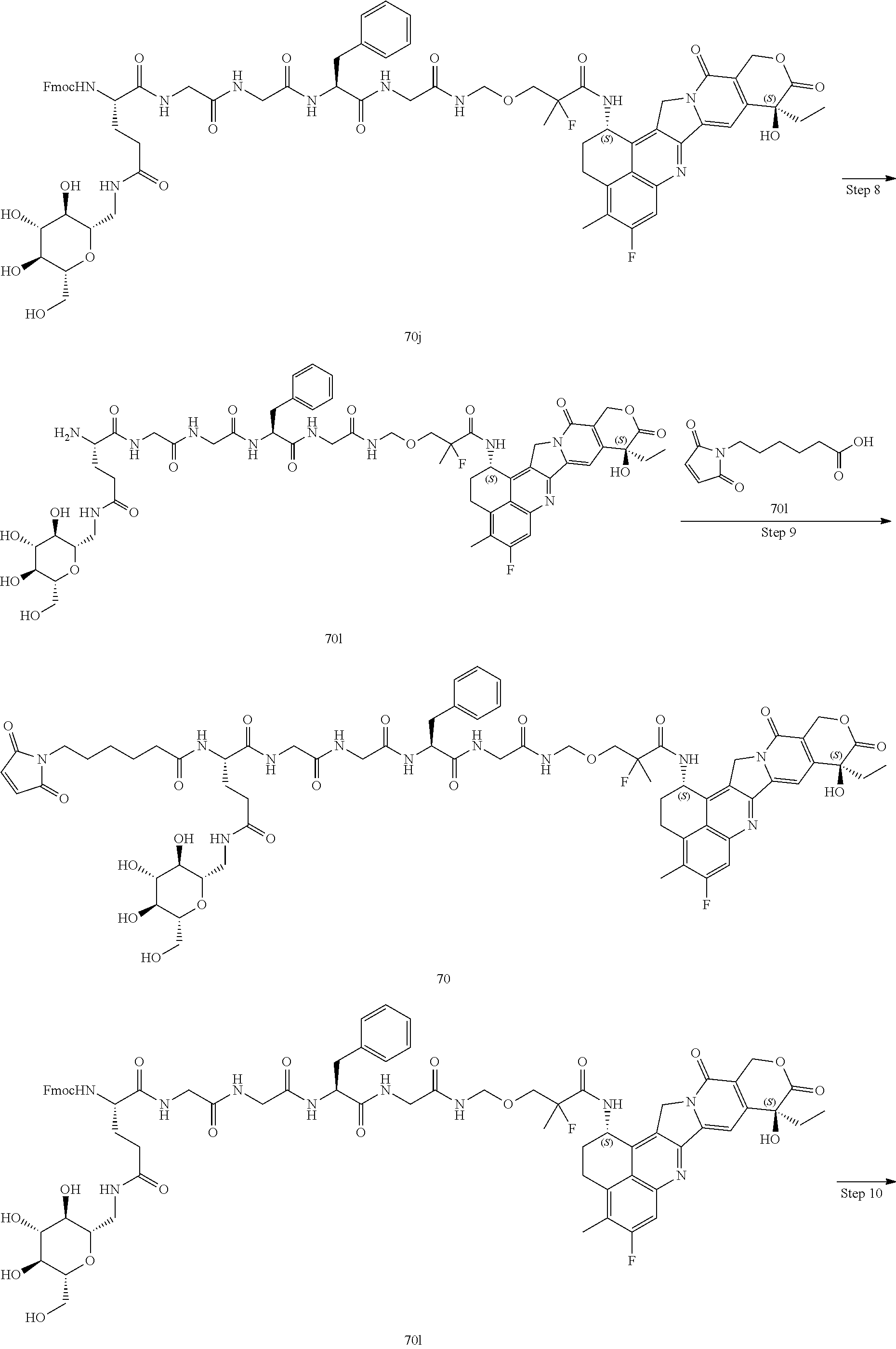

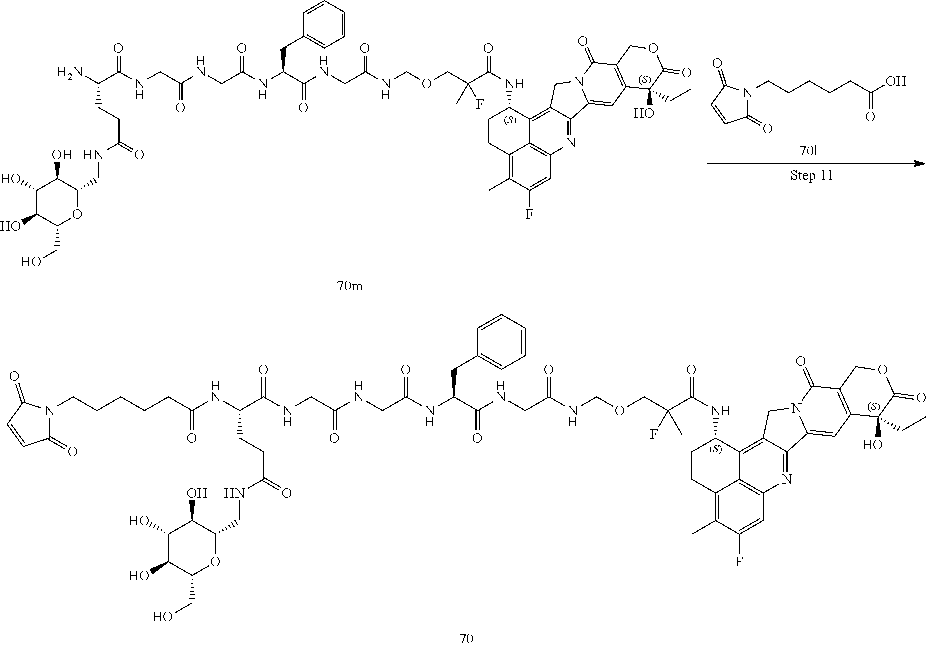

### Step 1

### benzyl 2-fluoro-3-hydroxy-2-methylpropanoate (70b)

To a solution of compound 70a (450 mg, 3.69 mmol) in DMF (10 mL) were added K₂CO₃ (1.02 g, 7.37 mmol) and BnBr (945 mg, 5.53 mmol). The mixture was stirred at 35° C. for 16 h. The mixture was diluted with EA (200 mL), washed by brine (50 mL*4). The organic layer was dried over anhydrous Na₂SO₄, filtered and concentrated. The residue was purified by silica column gel chromatography (eluent. PE/EA=100/0 to 30/70) to afford 70b (270 mg, 34.5% yield) as colorless oil.

¹H NMR (400 MHz, CDCl₃) δ 7.42-7.33 (m, 5H), 5.26 (s, 2H), 4.06-3.61 (m, 2H), 2.08 (br s, 1H), 1.55 (d, J=21.2 Hz, 3H).

### Step 2

### (S)-11-benzyl-1-(9H-fluoren-9-yl)-3,6,9,12,15-pentaoxo-2-oxa-4,7,10,13,16-pentaazaheptadecan-17-yl acetate (70d)

To a solution of compound 70c (5.00 g, 8.12 mmol) in DMF (65 mL) were added Cu(OAc)₂ (560 mg, 3.09 mmol), Pb(OAc)₄ (4.1 g, 9.25 mmol) and HOAc (1.11 g, 18.44 mmol). The mixture was stirred at 65° C. under N₂ atmosphere for 2 h. The mixture was concentrated to ¼ of the original volume, poured into ice-water (150 mL) and stirred for 30 min. The solid was filtered and co-evaporated with toluene (20 mL*4) to give compound 70d (5.20 g, crude) as off-white solid.

MS (ESI) m/z: 652.3 [M+Na]⁺

### Step 3

benzyl (11S)-11-benzyl-1-(9H-fluoren-9-yl)-20-fluoro-20-methyl-3,6,9,12,15-pentaoxo-2,18-dioxa-4,7,10,13,16-pentaazahenicosan-21-oate (70e)

To the mixture of 70d (250 mg, 0.40 mmol), 70b (253 mg, 1.19 mmol) and dried 4 Å molecular sieves (500 mg) in dry THE (8 mL) was added scandium trifluoromethanesulfonate (293 mg, 0.60 mmol), stirred at r.t. under N₂ atmosphere overnight. The solution was filtered through Celite, diluted with EA (100 mL), washed by saturated NaHCO₃ (40 mL*3). The organic layer was dried over anhydrous Na₂SO₄, filtered and concentrated. The residue was purified by silica column gel chromatography (eluent: PE/EA=100/0 to 50/50) to afford 70e (210 mg, 67.7% yield).

MS (ESI) m/z: 652.3 [M+Na]⁺

### Step 4

### benzyl (7S)-1-amino-7-benzyl-16-fluoro-16-methyl-2,5,8,11-tetraoxo-14-oxa-3,6,9,12-tetraazaheptadecan-17-oate (70f)

To a solution of compound 70e (210 mg, 0.27 mmol) in DMF (4 mL) was added Et₂NH (392 mg, 5.37 mmol). The mixture was stirred at r.t. for 30 min. The mixture was concentrated under high vacuum and co-evaporated with toluene (3 mL*3) to give 70f (210 mg, crude) as brown solid.

MS (ESI) m/z: 582.3 [M+Na]⁺

### Step 5

### benzyl (5S,14S)-14-benzyl-1-(9H-fluoren-9-yl)-23-fluoro-23-methyl-3,6,9,12,15,18-hexaoxo-5-(3-oxo-3-((((2S,3R,4R,5S,6R)-3,4,5-trihydroxy-6-(hydroxymethyl)tetrahydro-2H-pyran-2-yl)methyl)amino)propyl)-2,21-dioxa-4,7,10,13,16,19-hexaazatetracosan-24-oate (70 h)

To a solution of compound 70f (210 mg, crude) in DMF (4 mL) were added 70 g (161 mg, 0.29 mol), HATU (153 mg, 0.40 mmol) and DIEA (69 mg, 0.63 mmol). The mixture was stirred at r.t. for 30 min. The mixture was filtered and the filtrate was purified using prep-HPLC (Method: column: XBridge Prep C18 OBD Sum 19*250 mm; Mobile phase: A—water (0.1% formic acid): B—acetonitrile; Flow rate: 20 mL/min to provide 70 h (154 mg, 52.9% yield) as white solid.

MS (ESI) m/z: 1108.7 [M+Na]⁺

### Step 6

### (5S,14S)-14-benzyl-1-(9H-fluoren-9-yl)-23-fluoro-23-methyl-3,6,9,12,15,18-hexaoxo-5-(3-oxo-3-((((2S,3R,4R,5S,6R)-3,4,5-trihydroxy-6-(hydroxymethyl)tetrahydro-2H-pyran-2-yl)methyl)amino)propyl)-2,21-dioxa-4,7,10,13,16,19-hexaazatetracosan-24-oic acid (70i)

To a solution of compound 70h (154 mg, 0.14 mmol) in MeOH (6 mL) was added Pd/C (10%, 30 mg). The mixture was stirred at H₂ atmosphere (15 psi) for 4 h. The mixture was filtered through a pad of celite, concentrated to give compound 70i (141 mg, 100% yield) as white solid.

MS (ESI) m/z: 1018.6 [M+Na]⁺

### Step 7

### (9H-fluoren-9-yl)methyl ((6S,15S)-15-benzyl-25-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-24-fluoro-24-methyl-3,7,10,13,16,19,25-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazapentacosan-6-yl)carbamate (70j & 70k)

To a solution of compound 70i (141 mg, 0.15) in DMF (4 mL) were added 68e (86 mg, 0.16 mol), HATU (88 mg, 0.23 mmol) and DIEA (100 mg, 0.77 mmol). The mixture was stirred at r.t. for 30 min. The mixture was filtered and the filtrate was purified using prep-HPLC (Method: column: XBridge Prep C18 OBD Sum 19*250 mm; Mobile phase: A—water (0.1% trifluoroacetic acid): B—acetonitrile; Flow rate: 20 mL/min to provide two isomers:

Compound 70j (62 mg, 28.3% yield) was obtained as white solid.

MS (ESI) m/z: 1436.7 [M+Na]⁺

Compound 70k (64 mg, 29.7% yield) was obtained as white solid.

MS (ESI) m/z: 1436.7 [M+Na]⁺

### Step 8

### (2S)-2-amino-N1-((7S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16-fluoro-16-methyl-2,5,8,11,17-pentaoxo-14-oxa-3,6,9,12-tetraazaheptadecyl)-N5-(((2S,3R,4R,5S,6R)-3,4,5-trihydroxy-6-(hydroxymethyl)tetrahydro-2H-pyran-2-yl)methyl)pentanediamide (701)

To a solution of compound 70j (62 mg, 0.44 mmol) in DMF (4 mL) was added Et₂NH (64 mg, 0.88 mmol). The mixture was stirred at r.t. for 30 min. The mixture was concentrated under high vacuum and co-evaporated with toluene (3 mL*3) to give 701 (62 mg, crude) as brown solid.

MS (ESI) m/z: 1191.7 [M+Na]⁺

### Step 9

### (2S)—N1-((7S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16-fluoro-16-methyl-2,5,8,11,17-pentaoxo-14-oxa-3,6,9,12-tetraazaheptadecyl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (70)

To a solution of compound 701 (52 mg, 0.44 mmol) in DMF (3 mL) were added 70m (19 mg, 0.088 mol), HATU (33 mg, 0.088 mmol) and DIEA (11 mg, 0.088 mmol). The mixture was stirred at r.t. for 30 min. The mixture was filtered and the filtrate was purified using prep-HPLC (Method: column: XBridge Prep C18 OBD Sum 19*250 mm; Mobile phase: A—water (0.1% trifluoroacetic acid): B—acetonitrile; Flow rate: 20 mL/min to provide compound 70 (18.4 mg, 36.2% yield) as white solid.

¹H NMR (400 MHz, DMSO-d₆) δ 8.91 (d, J=9.2 Hz, 1H), 8.59 (t, J=6.8 Hz, 1H), 8.33 (t, J=5.8 Hz, 1H), 8.12 (d, J=7.2 Hz, 2H), 8.03 (t, J=7.6 Hz, 2H), 7.77 (d, J=10.8 Hz, 2H), 7.30 (s, 1H), 7.27-7.21 (m, 4H), 7.20-7.11 (m, 1H), 7.00 (d, J=8.4 Hz, 2H), 6.52 (s, 1H), 5.58 (d, J=7.8 Hz, 1H), 5.41 (s, 2H), 5.15 (dd, J=63.4, 18.8 Hz, 2H), 4.99 (d, J=5.4 Hz, 1H), 4.89 (dd, J=6.4, 5.2 Hz, 2H), 4.62 (d, J=6.8 Hz, 2H), 4.51 (dd, J=12.8, 9.2 Hz, 1H), 4.38 (dd, J=7.6, 4.6 Hz, 1H), 4.20 (d, J=6.0 Hz, 1H), 3.37 (dt, J=13.8, 6.8 Hz, 4H), 3.25 (d, J=18.0 Hz, 2H), 3.09 (ddd, J=20.8, 13.6, 9.2 Hz, 5H), 3.00-2.64 (m, 5H), 2.35 (d, J=18.8 Hz, 4H), 2.26-1.94 (m, 7H), 1.87 (dt, J=15.6, 7.2 Hz, 3H), 1.73 (d, J=8.0 Hz, 1H), 1.61 (d, J=21.8 Hz, 3H), 1.52-1.35 (m, 4H), 1.31-1.09 (m, 3H), 0.87 (t, J=7.2 Hz, 3H); MS (ESI) m/z: 1406.7 [M+Na]⁺; UPLC-MS Retention time: 3.99 min.

### Step 10

### (2S)-2-amino-N1-((7S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16-fluoro-16-methyl-2,5,8,11,17-pentaoxo-14-oxa-3,6,9,12-tetraazaheptadecyl)-N5-(((2S,3R,4R,5S,6R)-3,4,5-trihydroxy-6-(hydroxymethyl)tetrahydro-2H-pyran-2-yl)methyl)pentanediamide (70n)

To a solution of compound 70k (65 mg, 0.46 mmol) in DMF (4 mL) was added Et₂NH (67 mg, 0.92 mmol). The mixture was stirred at r.t. for 30 min. The mixture was concentrated under high vacuum and co-evaporated with toluene (3 mL*3) to give 70n (65 mg, crude) as brown solid.

MS (ESI) m/z: 1191.8 [M+Na]⁺

### Step 11

### (2S)—N1-((7S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16-fluoro-16-methyl-2,5,8,11,17-pentaoxo-14-oxa-3,6,9,12-tetraazaheptadecyl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (71)

To a solution of compound 70n (55 mg, 0.46 mmol) in DMF (3 mL) were added 70m (19 mg, 0.092 mol), HATU (35 mg, 0.092 mmol) and DIEA (12 mg, 0.092 mmol). The mixture was stirred at r.t. for 30 min. The mixture was filtered and the filtrate was purified using prep-HPLC (Method: column: XBridge Prep C18 OBD Sum 19*250 mm; Mobile phase: A—water (0.1% trifluoroacetic acid): B—acetonitrile; Flow rate: 20 mL/min to provide compound 71 (21 mg, 42.2% yield) as white solid.

¹H NMR (400 MHz, DMSO-d₆) δ 8.87 (d, J=6.4 Hz, 1H), 8.57 (t, J=6.8 Hz, 1H), 8.34 (t, J=6.0 Hz, 1H), 8.13 (t, J=7.2 Hz, 2H), 8.03 (d, J=7.6 Hz, 2H), 7.79 (t, J=9.6 Hz, 2H), 7.35 (s, 1H), 7.30-7.18 (m, 4H), 7.18-7.10 (m, 1H), 7.00 (d, J=1.6 Hz, 2H), 6.55 (s, 1H), 5.58 (d, J=8.0 Hz, 1H), 5.46 (q, J=16.4 Hz, 2H), 5.23 (q, J=19.2 Hz, 2H), 5.01 (d, J=5.4 Hz, 1H), 4.91 (dd, J=6.4, 5.2 Hz, 2H), 4.74 (ddd, J=29.2, 10.3, 6.8 Hz, 2H), 4.54 (dd, J=13.2, 8.8 Hz, 1H), 4.40 (dd, J=7.6, 4.4 Hz, 1H), 4.28-4.12 (m, 1H), 3.90 (dd, J=30.0, 11.2 Hz, 1H), 3.80-3.55 (m, 8H), 3.28-2.95 (m, 8H), 2.89 (td, J=9.0, 5.2 Hz, 2H), 2.78 (dd, J=13.8, 9.8 Hz, 1H), 2.37 (d, J=24.2 Hz, 4H), 2.25-2.04 (m, 6H), 1.99-1.80 (m, 3H), 1.80-1.68 (m, 1H), 1.58-1.37 (m, 8H), 1.36-1.08 (m, 3H), 0.89 (t, J=7.2 Hz, 3H); MS (ESI) m/z: 1406.7 [M+Na]⁺; UPLC-MS Retention time: 4.25 min.

### Example 72

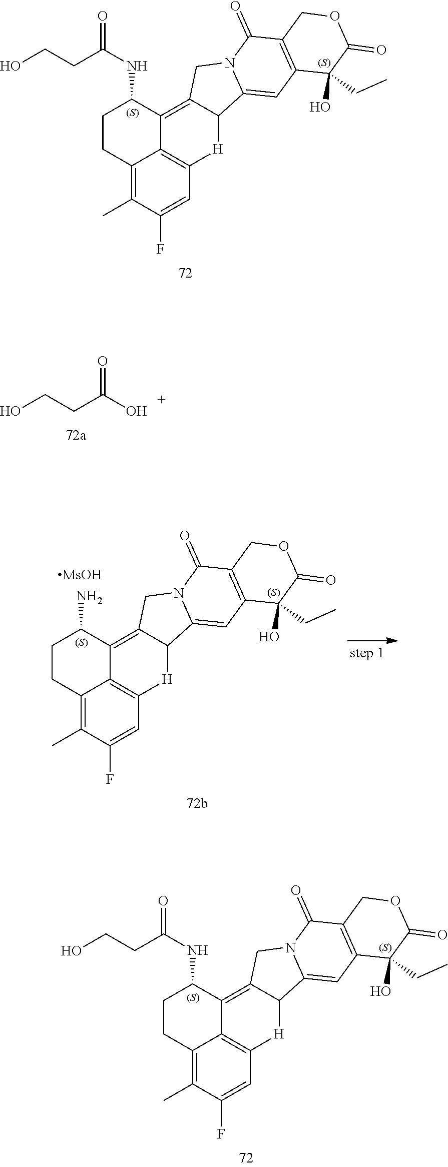

### Step 1

To a solution of compound 72b (50 mg, 0.094 mmol) in MeOH (2 mL) were added 72a (85 mg, 0.28 mmol, 30% in water), DMT-MM (78 mg, 0.28 mmol) and DIEA (36 mg, 0.28 mmol). The mixture was stirred at r.t. for 2 h. The mixture was filtered and the filtrate was purified using prep-HPLC (Method: column: XBridge Prep C18 OBD 5 μm 19*250 mm; Mobile phase: A—water (0.1% trifluoroacetic acid): B—acetonitrile; Flow rate: 20 mL/min to provide compound 72 (28.3 mg, 59.3% yield) as white solid.

¹H NMR (400 MHz, DMSO-d₆) δ 8.45 (d, J=8.8 Hz, 1H), 7.80 (d, J=11.2 Hz, 1H), 7.31 (s, 1H), 6.53 (s, 1H), 5.65-5.51 (m, 1H), 5.43 (s, 2H), 5.29-5.09 (m, 2H), 4.60 (t, J=5.2 Hz, 1H), 3.67 (dd, J=11.2, 5.6 Hz, 1H), 3.18 (s, 1H), 2.40 (s, 2H), 2.32 (t, J=6.4 Hz, 1H), 2.22-2.04 (m, 1H), 1.95-1.71 (m, 1H), 0.87 (t, J=7.2 Hz, 2H); MS (ESI) m/z: 508.3 [M+H]⁺; UPLC-MS Retention time: 3.28 min.

### Example 73

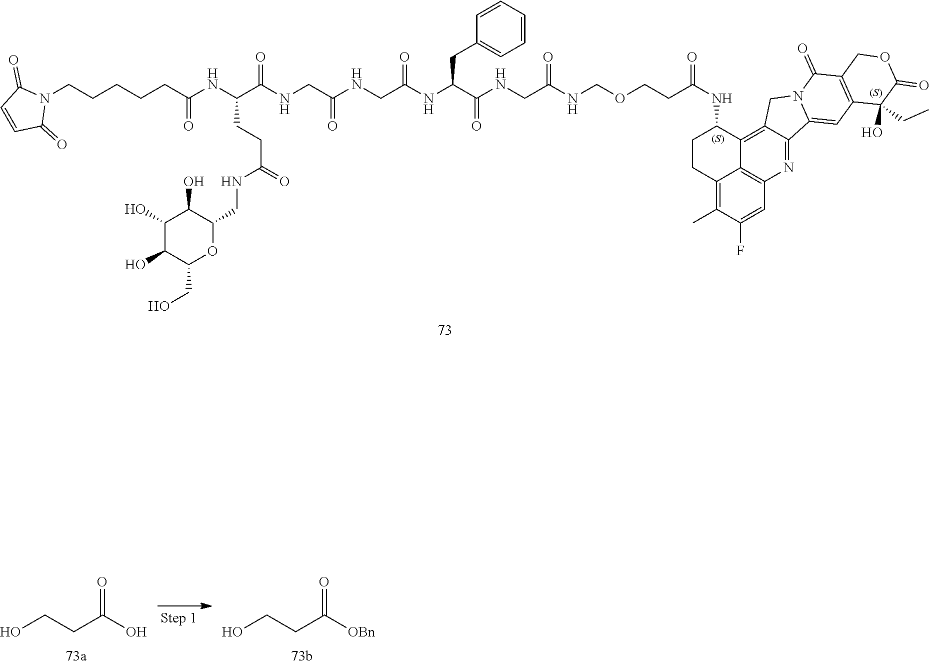

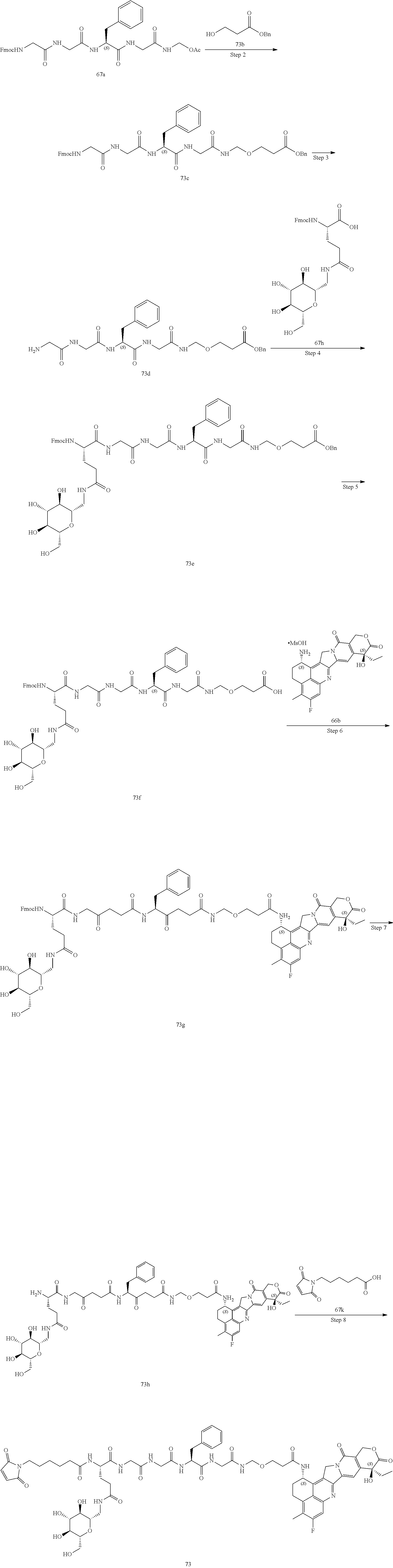

### Step 1

### benzyl 3-hydroxypropanoate (73b)

Compound 73a (1 g, 3.3 mmol, 30% in H₂O solution) and KOH (187 mg, 3.3 mmol) was stirred at rt for 30 min, the mixture was concentrated in vacuo to give a white solid. The solid was suspended in DMF (10 mL) was added BnBr (570 mg, 3.3 mmol) via a syringe at rt. After stirring at 80° C. for 5 h, the reaction mixture concentrated in vacuo. The residue was purified by a silica gel column chromatography (eluent: PE/EA=100/0 to 30/70) to give the title compound 73b (420 mg, 70% yield) colorless oil.

¹H NMR (400 MHz, CDCl₃) δ 7.39-7.33 (m, 5H), 5.16 (s, 2H), 3.88 (t, J=5.6H, 2H), 2.66 (t, J=5.6H, 2H), 2.38 (br s, 1H).

### Step 2

### benzyl(S)-11-benzyl-1-(9H-fluoren-9-yl)-3,6,9,12,15-pentaoxo-2,18-dioxa-4,7,10,13,16-pentaazahenicosan-21-oate (73c)

Compound 73c (1.2 g, 83.9% yield) was synthesized according to synthetic procedure of step 3 of example 70.

MS (ESI) m/z: 773.5 [M+Na]⁺

### Step 3

### Benzyl(S)-1-amino-7-benzyl-2,5,8,11-tetraoxo-14-oxa-3,6,9,12-tetraazaheptadecan-17-oate (73d)

Compound 73d (150 mg, crude) was synthesized according to synthetic procedure of step 4 of example 70.

MS (ESI) m/z: 528.3 [M+H]⁺

### Step 4

### benzyl(5S,14S)-14-benzyl-1-(9H-fluoren-9-yl)-3,6,9,12,15,18-hexaoxo-5-(3-oxo-3-((((2S,3R,4R,5S,6R)-3,4,5-trihydroxy-6-(hydroxymethyl)tetrahydro-2H-pyran-2-yl)methyl)amino)propyl)-2,21-dioxa-4,7,10,13,16,19-hexaazatetracosan-24-oate (73e)

Compound 73e (170 mg, 80.9% yield) was synthesized according to synthetic procedure of step 5 of example 70.

MS (ESI) m/z: 1076.6 [M+Na]⁺

### Step 5

### (5S,14S)-14-benzyl-1-(9H-fluoren-9-yl)-3,6,9,12,15,18-hexaoxo-5-(3-oxo-3-((((2S,3R,4R,5S,6R)-3,4,5-trihydroxy-6-(hydroxymethyl)tetrahydro-2H-pyran-2-yl)methyl)amino)propyl)-2,21-dioxa-4,7,10,13,16,19-hexaazatetracosan-24-oic acid (73f)

Compound 73f (150 mg, crude) was synthesized according to synthetic procedure of step 6 of example 70.

MS (ESI) m/z: 986.5 [M+Na]⁺

### Step 6

### (9H-fluoren-9-yl)methyl ((6S,15S)-15-benzyl-25-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-3,7,10,13,16,19,25-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazapentacosan-6-yl)carbamate (73 g)

Compound 73g (190 mg, 88.4% yield) was synthesized according to synthetic procedure of step 7 of example 70.

MS (ESI) m/z: 1404.5 [M+Na]⁺

### Step 7

### (S)-2-amino-N1-((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,5,8,11,17-pentaoxo-14-oxa-3,6,9,12-tetraazaheptadecyl)-N5-(((2S,3R,4R,5S,6R)-3,4,5-trihydroxy-6-(hydroxymethyl)tetrahydro-2H-pyran-2-yl)methyl)pentanediamide (73 h)

Compound 73g (190 mg, crude) was synthesized according to synthetic procedure of step 8 of example 70.

### Step 8

### (S)—N1-((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,5,8,11,17-pentaoxo-14-oxa-3,6,9,12-tetraazaheptadecyl)-2-(6-(2,5-dioxo-2,5-dihydro-1H-pyrrol-1-yl)hexanamido)-N5-(((2S,3R,4R,5S,6R)-3,4,5-trihydroxy-6-(hydroxymethyl)tetrahydro-2H-pyran-2-yl)methyl)pentanediamide (73)

Compound 73 (50.3 mg, 54.1% yield) was synthesized according to synthetic procedure of step 9 of example 70.

¹H NMR (400 MHz, DMSO-d₆) δ 8.51 (d, J=9.2 Hz, 2H), 8.32 (s, 1H), 8.13 (d, J=8.0 Hz, 2H), 8.03 (d, J=7.2 Hz, 2H), 7.80 (d, J=11.2 Hz, 2H), 7.32 (s, 1H), 7.28-7.19 (m, 4H), 7.17 (d, J=6.6 Hz, 1H), 7.00 (d, J=8.0 Hz, 2H), 6.54 (s, 1H), 5.56 (d, J=8.4 Hz, 1H), 5.43 (s, 2H), 5.22 (d, J=4.8 Hz, 2H), 5.01 (d, J=5.2 Hz, 1H), 4.94-4.84 (m, 2H), 4.65-4.44 (m, 3H), 4.43-4.33 (m, 1H), 4.19 (d, J=6.0 Hz, 1H), 3.84-3.51 (m, 10H), 3.22-2.93 (m, 8H), 2.88 (s, 2H), 2.83-2.70 (m, 1H), 2.37 (d, J=29.2 Hz, 6H), 2.10 (dd, J=14.6, 7.3 Hz, 7H), 1.97-1.78 (m, 3H), 1.72 (d, J=8.0 Hz, 1H), 1.59-1.37 (m, 4H), 1.22-1.10 (m, 2H), 0.87 (t, J=7.2 Hz, 3H).

MS (ESI) m/z: 1374.9 [M+Na]⁺; UPLC-MS Retention time: 3.84 min.

### Example 74

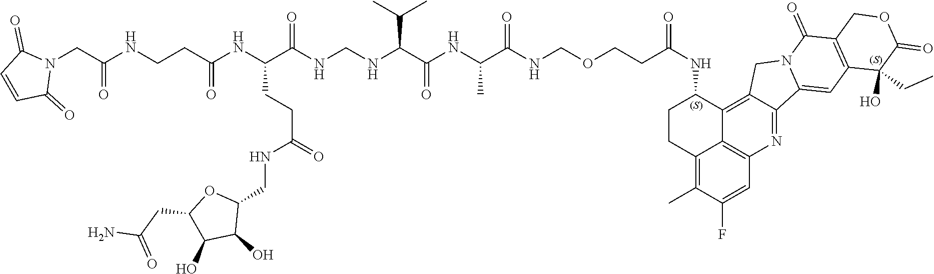

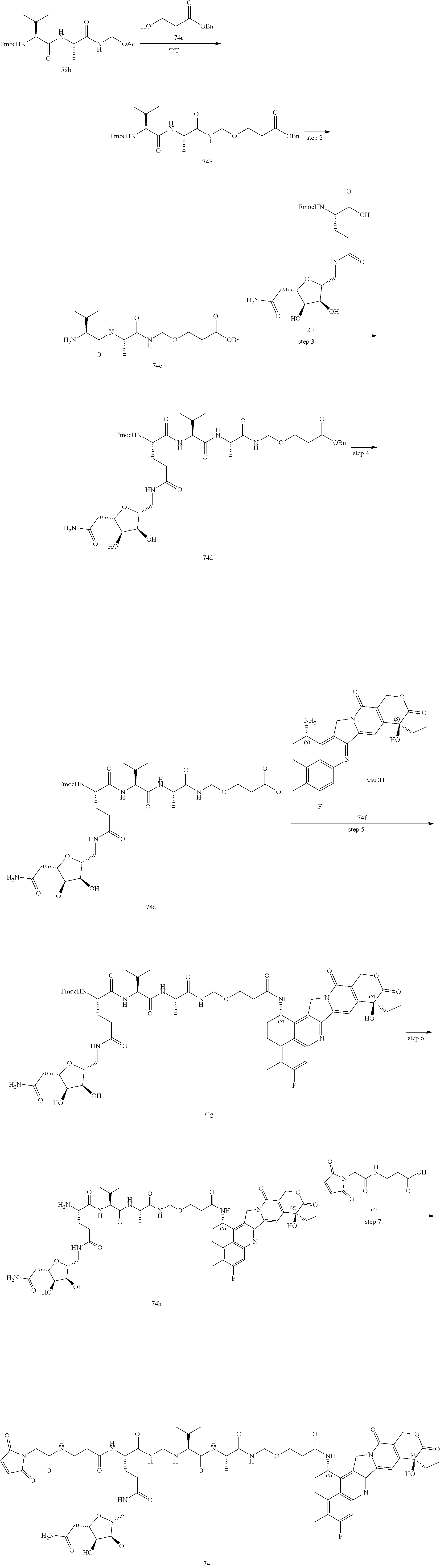

### Step 1

### benzyl(5S,8S)-1-(9H-fluoren-9-yl)-5-isopropyl-8-methyl-3,6,9-trioxo-2,12-dioxa-4,7,10-triazapentadecan-15-oate (74b)

To the mixture of 58b (750 mg, 1.59 mmol), 74a (842 mg, 4.68 mmol) and dried 4 Å molecular sieves (2000 mg) in dry THE (25 mL) was added scandium trifluoromethanesulfonate (938 mg, 1.91 mmol), stirred at r.t. under N₂ atmosphere overnight. The solution was filtered through Celite, diluted with EA (100 mL), washed by saturated NaHCO₃ (60 mL*3). The organic layer was dried over anhydrous Na₂SO₄, filtered and concentrated. The residue was purified by silica column gel chromatography (eluent: DCM/MeOH=100/0 to 50/50) to afford 74b (720 mg, 75.3% yield).

MS (ESI) m/z: 624.2 [M+Na]⁺

### Step 2

### benzyl3-(((S)-2-((S)-2-amino-3-methylbutanamido)propanamido)methoxy)propanoate (74c)

To a solution of compound 74b (140 mg, 0.23 mmol) in DMF (2 mL) was added Et₂NH (252 mg, 3.45 mmol). The mixture was stirred at r.t. for 30 min. The mixture was concentrated under high vacuum and co-evaporated with toluene (3 mL*3) to give 74c (87 mg, crude) as brown solid.

MS (ESI) m/z: 402.2 [M+Na]⁺

### Step 3

### benzyl (5S,8S,11S)-5-(3-((((2R,3S,4R,5S)-5-(2-amino-2-oxoethyl)-3,4-dihydroxytetrahydrofuran-2-yl)methyl)amino)-3-oxopropyl)-1-(9H-fluoren-9-yl)-8-isopropyl-11-methyl-3,6,9,12-tetraoxo-2,15-dioxa-4,7,10,13-tetraazaoctadecan-18-oate (74d)

To a solution of compound 74c (87 mg, crude) in DMF (3 mL) were added 20 (124 mg, 0.23 mol), HATU (96 mg, 0.27 mmol) and DIEA (59 mg, 0.46 mmol). The mixture was stirred at r.t. for 30 min. The mixture was filtered and the filtrate was purified using prep-HPLC (Method: column: XBridge Prep C18 OBD Sum 19*250 mm; Mobile phase: A—water (0.1% formic acid): B—acetonitrile; Flow rate: 20 mL/min to provide 74d (95 mg, 45.7% yield) as white solid.

MS (ESI) m/z: 925.3 [M+Na]⁺

### Step 4

### (5S,8S,11S)-5-(3-((((2R,3S,4R,5S)-5-(2-amino-2-oxoethyl)-3,4-dihydroxytetrahydrofuran-2-yl)methyl)amino)-3-oxopropyl)-1-(9H-fluoren-9-yl)-8-isopropyl-11-methyl-3,6,9,12-tetraoxo-2,15-dioxa-4,7,10,13-tetraazaoctadecan-18-oic acid (74e)

To a solution of compound 74d (95 mg, 0.11 mmol) in MeOH (15 mL) and DMF (3 mL) was added Pd/C (10%, 15 mg). The mixture was stirred at H₂ atmosphere (15 psi) for 4 h. The mixture was filtered through a pad of celite, concentrated to give compound 74e (85 mg, crude) as white solid.

MS (ESI) m/z: 835.3 [M+Na]⁺

### Step 5

### (9H-fluoren-9-yl)methyl ((6S,9S,12S)-1-((2R,3S,4R,5S)-5-(2-amino-2-oxoethyl)-3,4-dihydroxytetrahydrofuran-2-yl)-19-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-9-isopropyl-12-methyl-3,7,10,13,19-pentaoxo-16-oxa-2,8,11,14-tetraazanonadecan-6-yl)carbamate (74 g)

To a solution of compound 74e (85 mg, crude) in DMF (3 mL) were added 74f (61 mg, 0.12 mol), HATU (47.4 mg, 0.13 mmol) and DIEA (27 mg, 0.21 mmol). The mixture was stirred at r.t. for 30 min. The mixture was filtered and the filtrate was purified using prep-HPLC (Method: column: XBridge Prep C18 OBD 5 μm 19*250 mm; Mobile phase: A—water (0.1% formic acid): B—acetonitrile; Flow rate: 20 mL/min to provide 74 g (50 mg, 39.1% yield) as yellow solid.

MS (ESI) m/z: 1252.5 [M+Na]⁺

### Step 6

### (S)-2-amino-N5-(((2R,3S,4R,5S)-5-(2-amino-2-oxoethyl)-3,4-dihydroxytetrahydrofuran-2-yl)methyl)-N1-((S)-1-(((S)-1-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-3-oxopropoxy)methyl)amino)-1-oxopropan-2-yl)amino)-3-methyl-1-oxobutan-2-yl)pentanediamide (74 h)

To a solution of compound 74g (50 mg, 0.04 mmol) in DMF (2 mL) was added Et₂NH (45 mg, 0.61 mmol). The mixture was stirred at r.t. for 30 min. The mixture was concentrated under high vacuum and co-evaporated with toluene (3 mL*3) to give 74 h (40 mg, crude) as brown solid.

MS (ESI) m/z: 1030.4 [M+Na]⁺

### Step 7

### (S)—N5-(((2R,3S,4R,5S)-5-(2-amino-2-oxoethyl)-3,4-dihydroxytetrahydrofuran-2-yl)methyl)-2-(3-(2-(2,5-dioxo-2,5-dihydro-1H-pyrrol-1-yl)acetamido)propanamido)-N1-((S)-1-(((S)-1-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-3-oxopropoxy)methyl)amino)-1-oxopropan-2-yl)amino)-3-methyl-1-oxobutan-2-yl)pentanediamide (74)

To a solution of compound 74h (40 mg, crude) in DMF (2 mL) were added 74i (11.7 mg, 0.05 mol), HATU (19 mg, 0.05 mmol) and DIEA (12 mg, 0.09 mmol). The mixture was stirred at r.t. for 30 min. The mixture was filtered and the filtrate was purified using prep-HPLC (Method: column: XBridge Prep C18 OBD Sum 19*250 mm; Mobile phase: A—water (0.1% trifluoroacetic acid): B—acetonitrile; Flow rate: 20 mL/min to provide 74

(29 mg, 59.6% yield) was obtained as white solid.

MS (ESI) m/z: 1238.7 [M+Na]⁺; UPLC-MS Retention time: 3.39 min.

### ADC Preparation and Characterization

### Antibody Drug Conjugate Preparation

### DAR8 Antibody Drug Conjugate Preparation

Antibody in conjugation buffer (with concentration 0.5-25 mg/mL, PBS buffer pH 6.0-8.5) was incubated under reduction temperature (0-40° C.) for 10 min and 8-15 eq. TECP solution (5 mM stock in PBS buffer) was added into the reaction mixture and left the reduction reaction for 1-8 hours at reduction temperature. Organic solvent (eg: DMSO, DMF, DMA, PG, acetonitrile, 0-25% v/v) and linker-payload stock (10-25 eq, 10 mM stock in organic solvent) were added stepwise after reduction mixture was cooled down to 0-25° C. Conjugation solution was left for 1-3 h at 0-25° C. and the reaction can be quenched with N-acetyl Cysteine (1 mM stock). The solution was submitted to buffer exchange (spin desalting column, ultrafiltration, and dialysis) into storage buffer (for example: pH 5.5-6.5 Histidine acetate buffer, with optional additive such as sucrose, trehalose, tween 20, 60, 80).

### ADC Characterization

ADC examples in this invention were prepared by following above procedures with DAR 8 profile. All ADCs in this invention were characterized via following analytical methods.

Drug to antibody ratio (DAR) of the ADCs in this invention were determined by LCMS method or HIC method.

SEC purity of ADCs made in this invention are all >95% purity.

### Drug to Antibody Ratio (DAR) Determination

### LCMS Method

LC-MS analysis was carried out under the following measurement conditions:
LC-MS system: Vanquish Flex UHPLC and Orbitrap Exploris 240 Mass Spectrometer
Column: MAbPac™ RP, 2.1*50 mm, 4 μm, 1,500 Å, Thermo Scientific™
Column temperature: 80° C.
Mobile phase A: 0.1% formic acid (FA) aqueous solution
Mobile phase B: Acetonitrile solution containing 0.1% formic acid (FA)
Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)
Injected sample amount: 1 μg

MS parameters: Intact and denaturing MS data were acquired in HMR mode at setting of R=15k and deconvolved using the ReSpect™ algorithm and Sliding Window integration in Thermo Scientific™ BioPharma Finder™ 4.0 software.

### HIC Method

HPLC analysis was carried out under the following measurement conditions:
HPLC system: Waters ACQUITY ARC HPLC System
Detector: measurement wavelegth: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM Phosphate buffer, pH 7.0
Mobile phase B: 50 mM Phosphate buffer, 25% (V/V) Isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 g

### ADC Purity

### SEC Method

HPLC analysis was carried out under the following measurement conditions:
HPLC system: Waters H-Class UPLC System
Detector: measurement wavelegth: 280 nm
Column: ACQUITY UPLC BEH200 SEC 1.7 um 4.6×150 mm, Waters
Column temperature: room temperature
Mobile phase A: 200 mM Phosphate buffer, 250 mM potassium chloride, 15% isopropyl alcohol, PH 7.0
Gradient program: under 10 min isocratic elutions with the flow rate of 0.3 mL/min
Injected sample amount: 20 μg

### ADC Hydrophobicity Evaluation

ADC with more hydrophobic property would appeared with later retention time from HIC (hydrophobicity interaction column) chromatography. In this invention, the DAR8 (antibody with 8 drug-loading) peak of the example ADCs for this comparison.

By comparing HIC D8 RT, it is clearly to tell that ADC hydrophilicity ranks as below ADC-C4>ADC-C5>ADC-C2>ADC-C3>ADC-C1. ADC-C1 appears as the most hydrophobic property in this set.

### HIC Method

### Method 1

HPLC analysis was carried out under the following measurement conditions:
HPLC system: Waters ACQUITY ARC HPLC System
Detector: measurement wavelegth: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM Phosphate buffer, pH 7.0
Mobile phase B: 50 mM Phosphate buffer, 25% (V/V) Isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 g

### Method 2

HPLC analysis was carried out under the following measurement conditions:
HPLC system: Waters ACQUITY ARC HPLC System
Detector: measurement wavelegth: 280 nm
Column: MABPac HIC-10, 5 μm, 4.6×10 mm (Thermo)
Column temperature: 25° C.
Mobile phase A: 1.5 M ammonium sulfate, 50 mM sodium phosphate, pH 7.0
Mobile phase B: 50 mM sodium phosphate, pH 7.0
Gradient program: 20% B—20% B (0 min-1 min), 0O % B-0% B (1 min-35 min), 20% B-20% B (35 min-40 min)
Flow rate: 0.5 mL/min
Sample preparation: The sample was diluted with initial mobile phase to 0.5 mg/mL.

Examples of Linker plus payload are provided in the following table:

TABLE 1

| Com- | |
|---|---|
| pound | |
| No. | Linker-payload Structure |
| 2 | 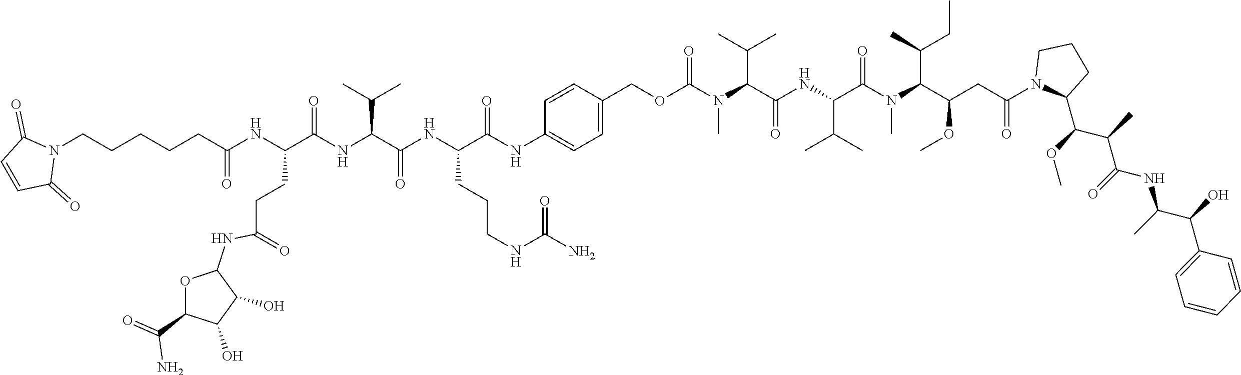 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)NC2O[C@H](C(N)=O)[C@@H](O)[C@H]2O)NC(=O)CCCCCN2C(=O)C=CC2=O)C(C)C)cc1)C(C)C] |
| | 2 |
| 4 | 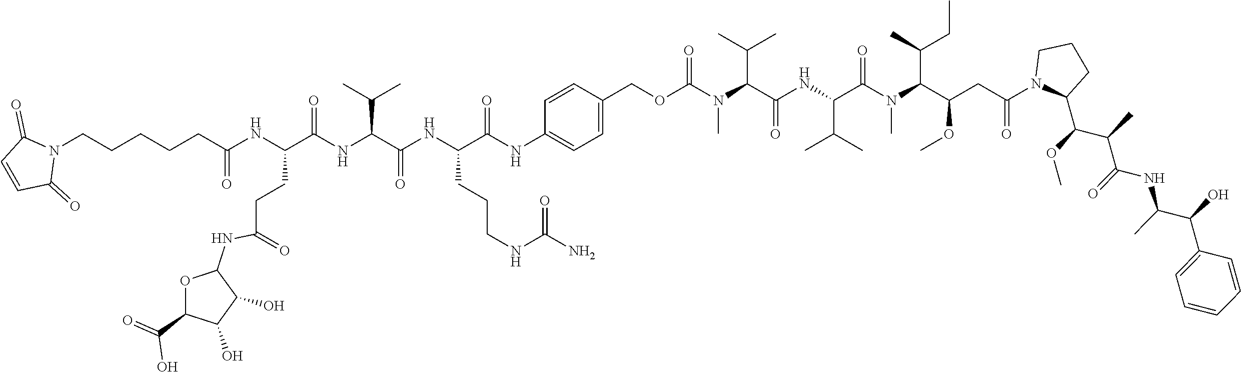 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)NC2O[C@H](C(=O)O)[C@@H](O)[C@H]2O)NC(=O)CCCCCN2C(=O)C=CC2=O)C(C)C)cc1)C(C)C] |
| | 4 |
| 6 | 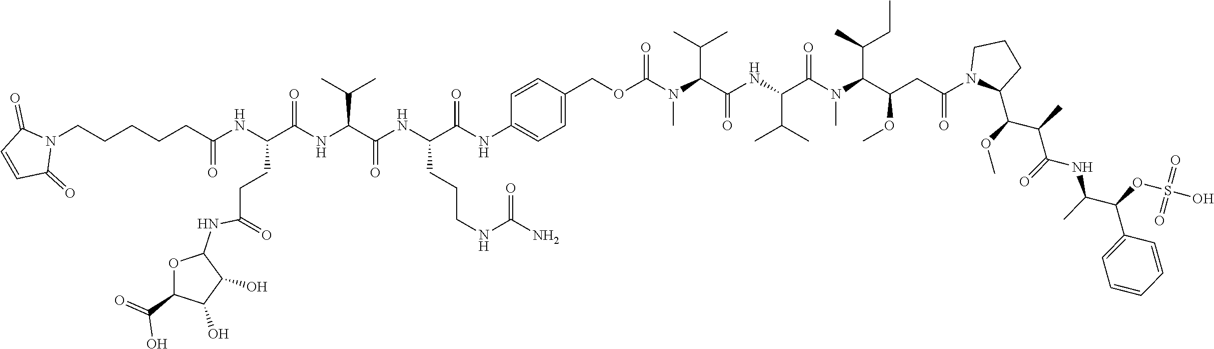 [SMILES: CC[C@H](C)[C@@H]([C@@H](CC(=O)N1CCC[C@H]1[C@H](OC)[C@@H](C)C(=O)N[C@H](C)[C@@H](OS(=O)(=O)O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)NC2O[C@H](C(=O)O)[C@@H](O)[C@H]2O)NC(=O)CCCCCN2C(=O)C=CC2=O)C(C)C)cc1)C(C)C] |
| | 6 |
| 7 | 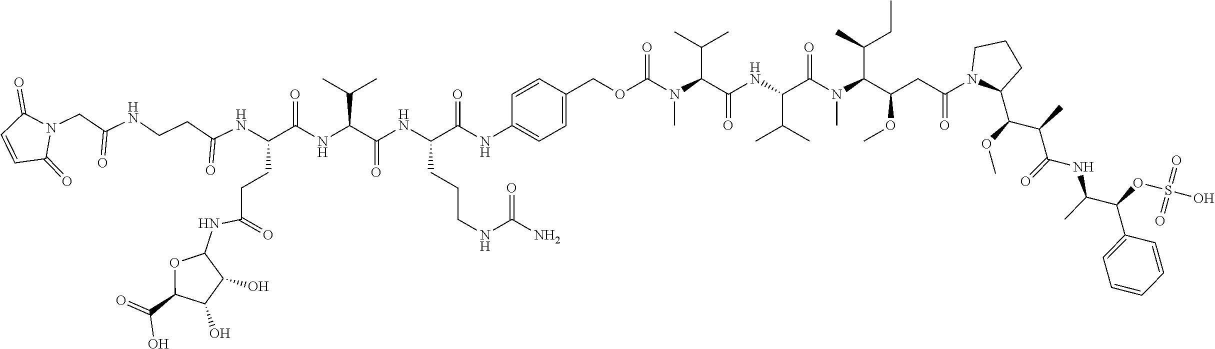 [SMILES: CC[C@H](C)[C@@H]([C@@H](CC(=O)N1CCC[C@H]1[C@H](OC)[C@@H](C)C(=O)N[C@H](C)[C@@H](OS(=O)(=O)O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)NC2O[C@H](C(=O)O)[C@@H](O)[C@H]2O)NC(=O)CCNC(=O)CN2C(=O)C=CC2=O)C(C)C)cc1)C(C)C] |
| | 7 |
| 9 | 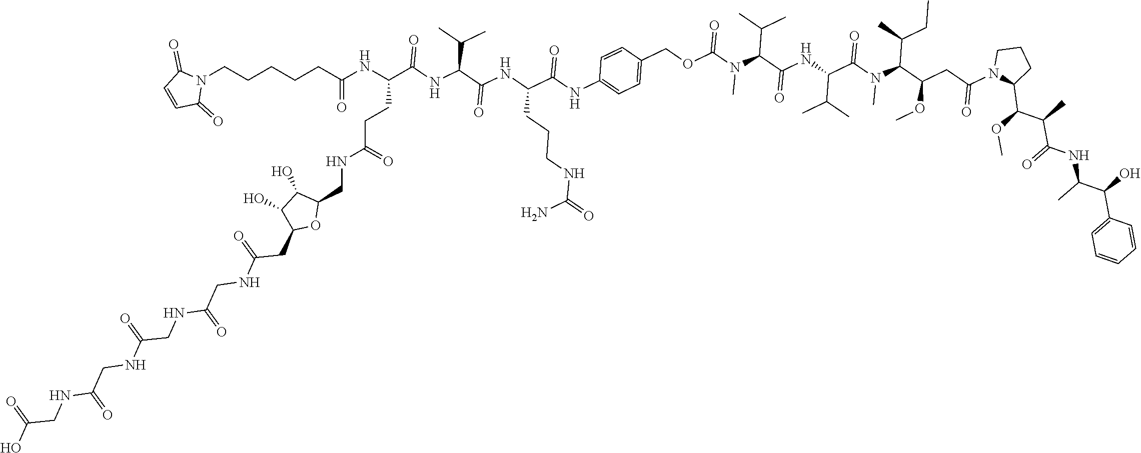 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)NC[C@H]2O[C@@H](CC(=O)NCC(=O)NCC(=O)NCC(=O)NCC(=O)O)[C@H](O)[C@@H]2O)NC(=O)CCCCCN2C(=O)C=CC2=O)C(C)C)cc1)C(C)C] |
| | 9 |
| 10 | 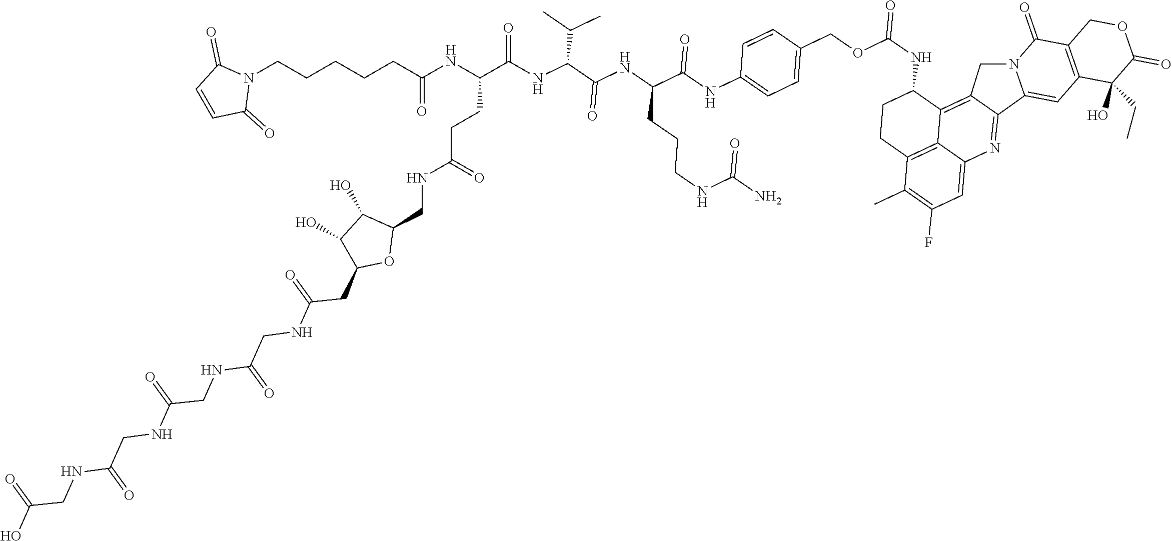 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@@H](CCCNC(N)=O)NC(=O)[C@H](NC(=O)[C@H](CCC(=O)NC[C@H]2O[C@@H](CC(=O)NCC(=O)NCC(=O)NCC(=O)NCC(=O)O)[C@H](O)[C@@H]2O)NC(=O)CCCCCN2C(=O)C=CC2=O)C(C)C)cc1)CC3] |
| 11 | 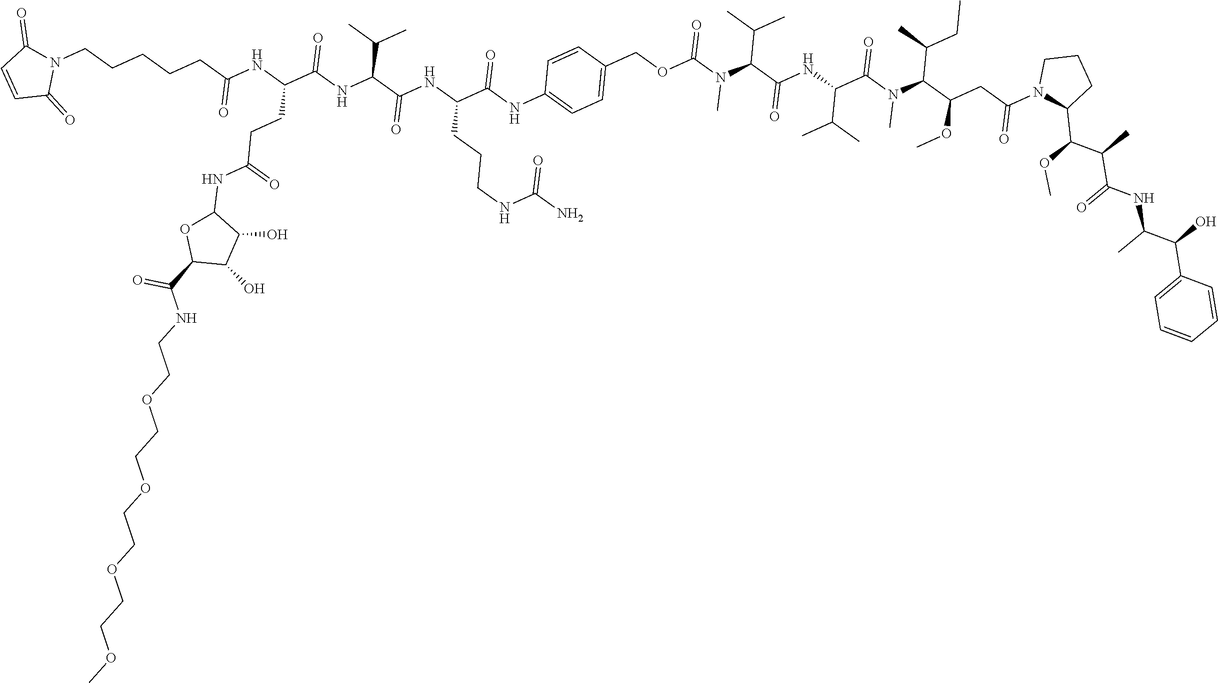 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)NC2O[C@H](C(=O)NCCOCCOCCOCCOC)[C@@H](O)[C@H]2O)NC(=O)CCCCCN2C(=O)C=CC2=O)C(C)C)cc1)C(C)C] |
| | 11 |
| 12 | 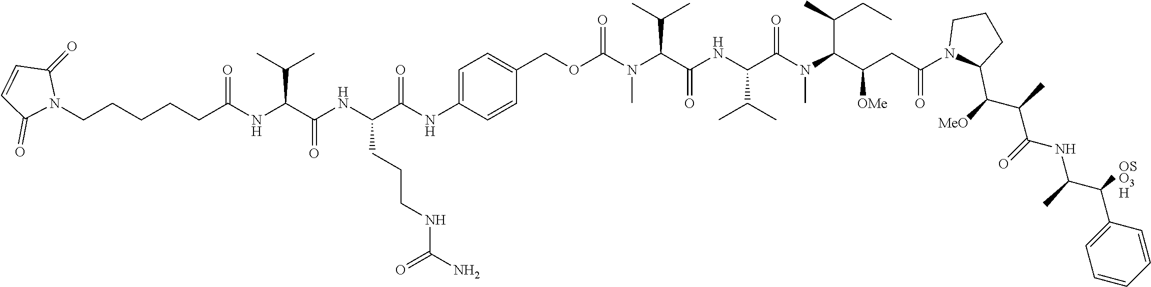 |
| | 12 |
| 13 | 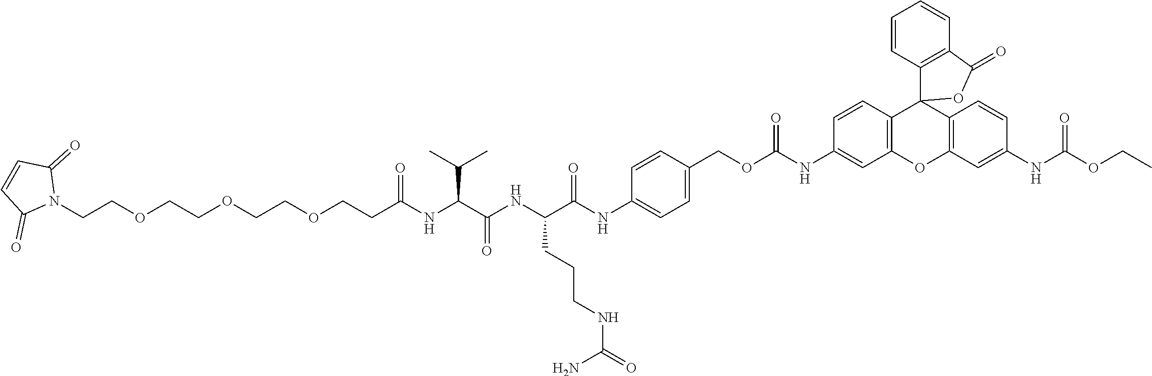 [SMILES: CCOC(=O)Nc1ccc2c(c1)Oc1cc(NC(=O)OCc3ccc(NC(=O)[C@H](CCCNC(N)=O)NC(=O)[C@@H](NC(=O)CCOCCOCCOCCN4C(=O)C=CC4=O)C(C)C)cc3)ccc1C21OC(=O)c2ccccc21] |
| | 13 |
| 14 | 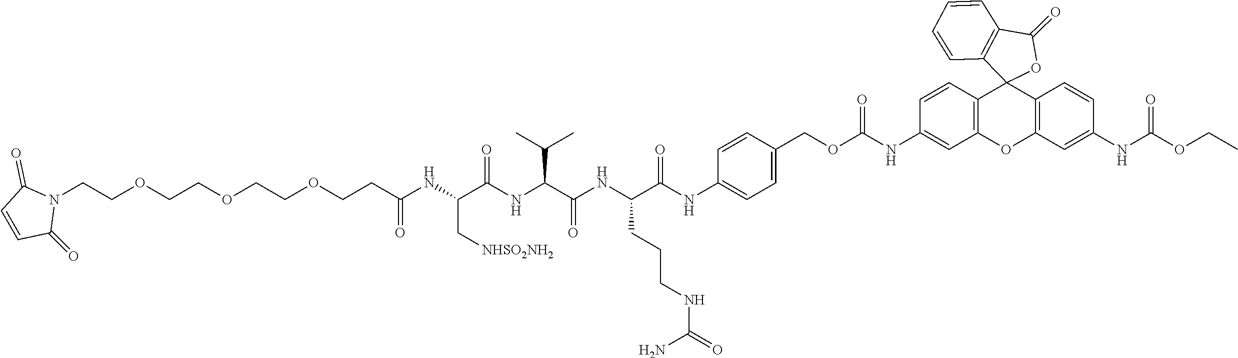 |
| | 14 |
| 15 | 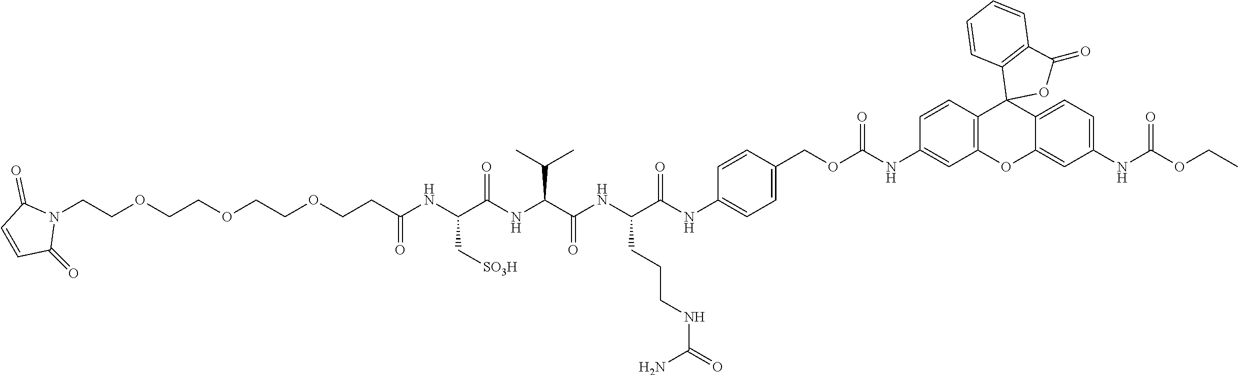 [SMILES: CCOC(=O)Nc1ccc2c(c1)Oc1cc(NC(=O)OCc3ccc(NC(=O)[C@H](CCCNC(N)=O)NC(=O)[C@@H](NC(=O)[C@H](CS(=O)(=O)O)NC(=O)CCOCCOCCOCCN4C(=O)C=CC4=O)C(C)C)cc3)ccc1C21OC(=O)c2ccccc21] |
| | 15 |
| 16 | 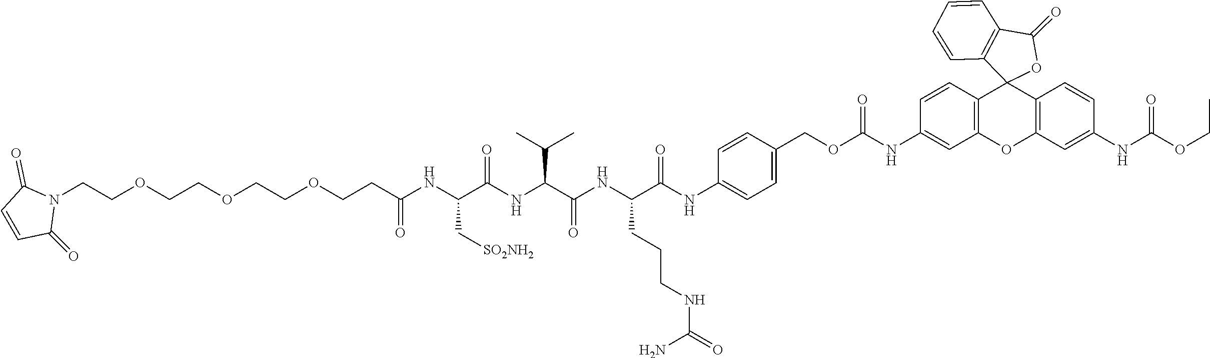 [SMILES: CCOC(=O)Nc1ccc2c(c1)Oc1cc(NC(=O)OCc3ccc(NC(=O)[C@H](CCCNC(N)=O)NC(=O)[C@@H](NC(=O)[C@H](CS(N)(=O)=O)NC(=O)CCOCCOCCOCCN4C(=O)C=CC4=O)C(C)C)cc3)ccc1C21OC(=O)c2ccccc21] |
| | 16 |
| 17 | 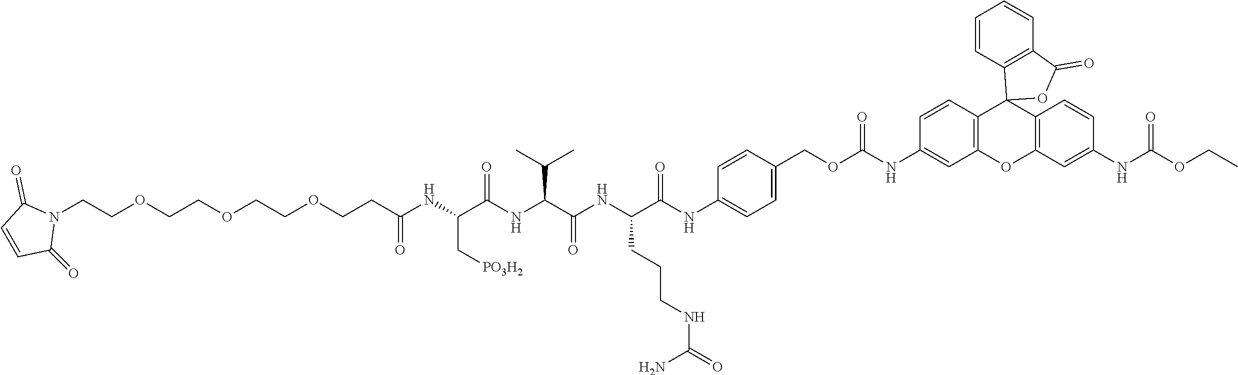 |
| | 17 |
| 22 | 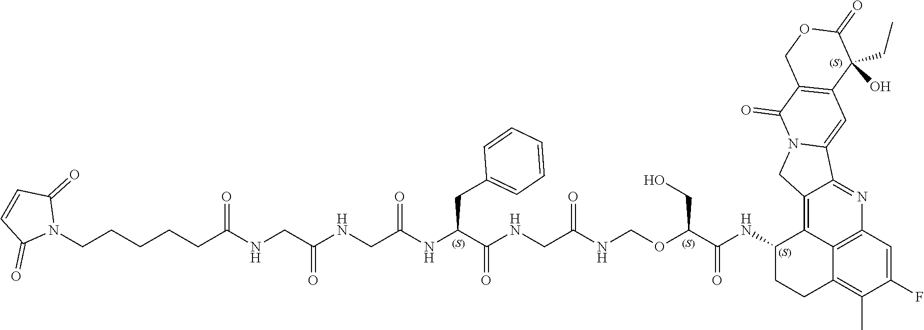 |
| | 22 |
| 23 | 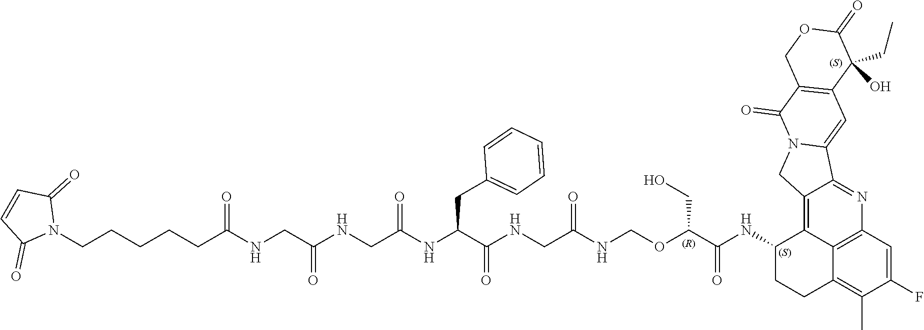 |
| | 23 |
| 24 | 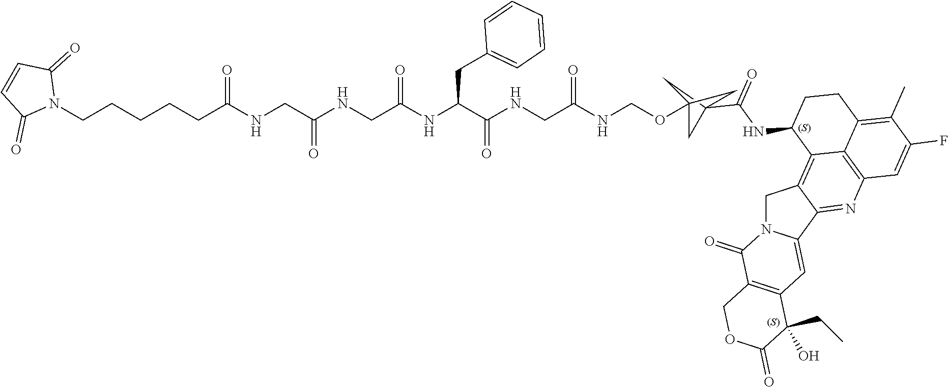 |
| | 24 |
| 25 | 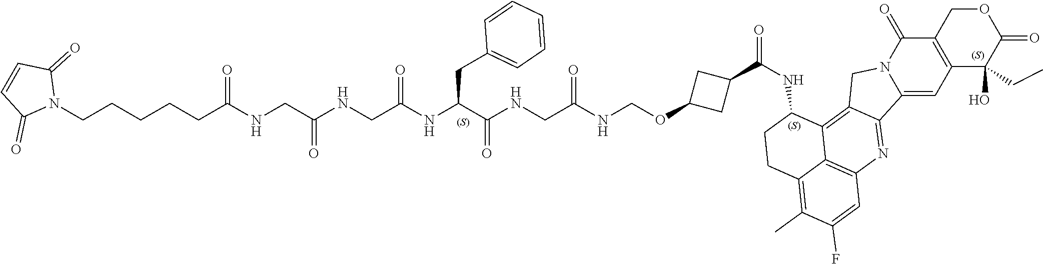 |
| | 25 |
| 26 | 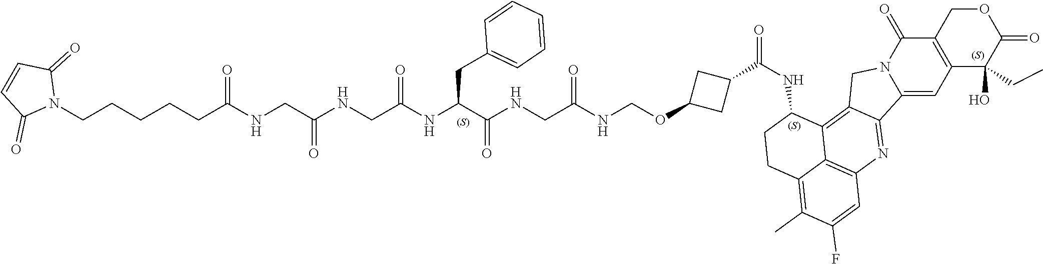 |
| | 26 |
| 27 | 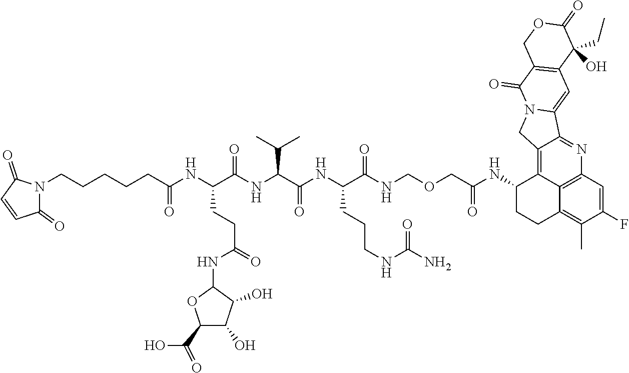 [SMILES: CC[C@@]1(O)C(=O)OCc2c1cc1n(c2=O)Cc2c-1nc1cc(F)c(C)c3c1c2[C@@H](NC(=O)COCNC(=O)[C@H](CCCNC(N)=O)NC(=O)[C@@H](NC(=O)[C@H](CCC(=O)NC1O[C@H](C(=O)O)[C@@H](O)[C@H]1O)NC(=O)CCCCCN1C(=O)C=CC1=O)C(C)C)CC3] |
| | 27 |
| 29 | 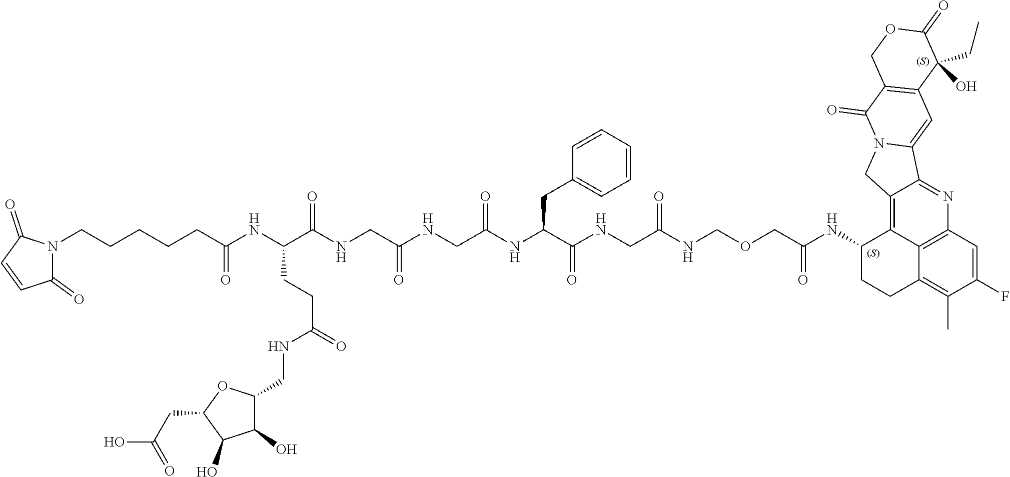 |
| | 29 |
| 30 | 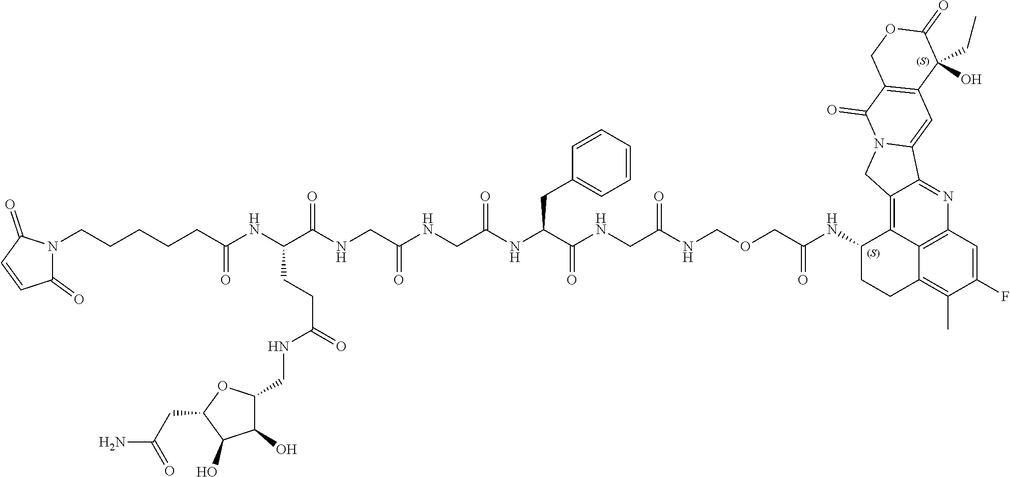 |
| | 30 |
| 31 | 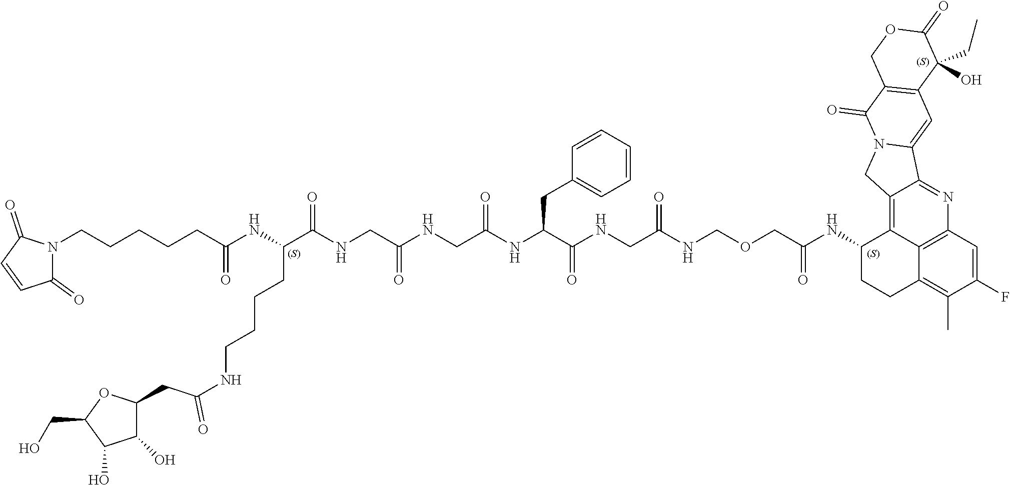 |
| | 31 |
| 32 | 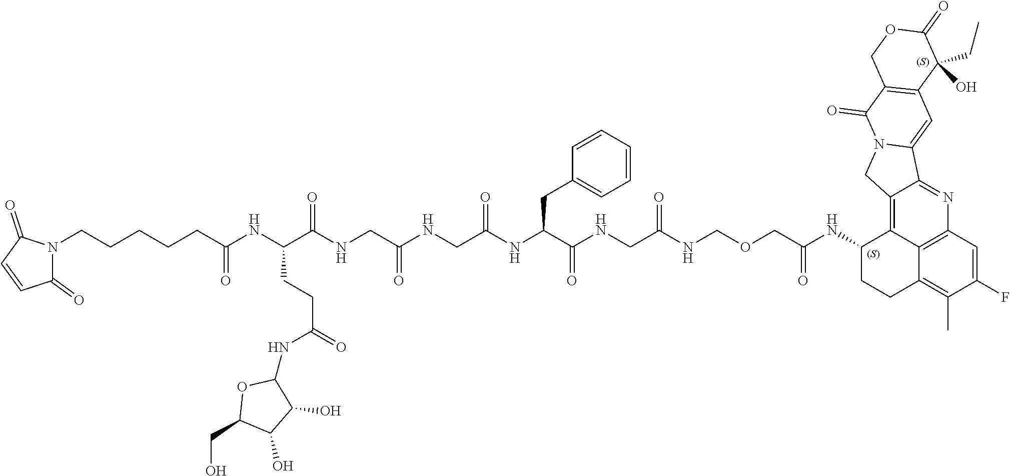 |
| | 32 |
| 33 | 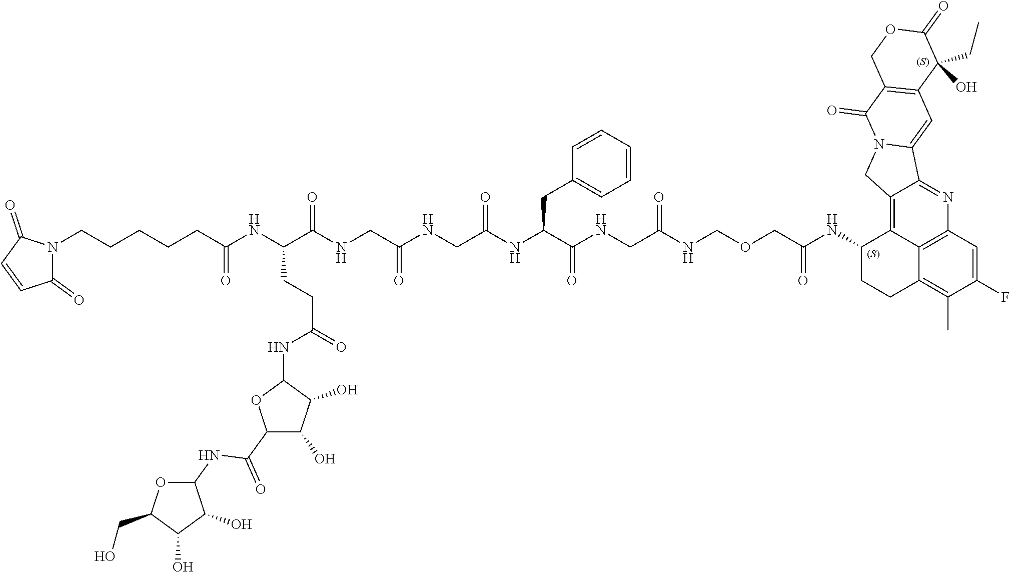 |
| | 33 |
| 34 | 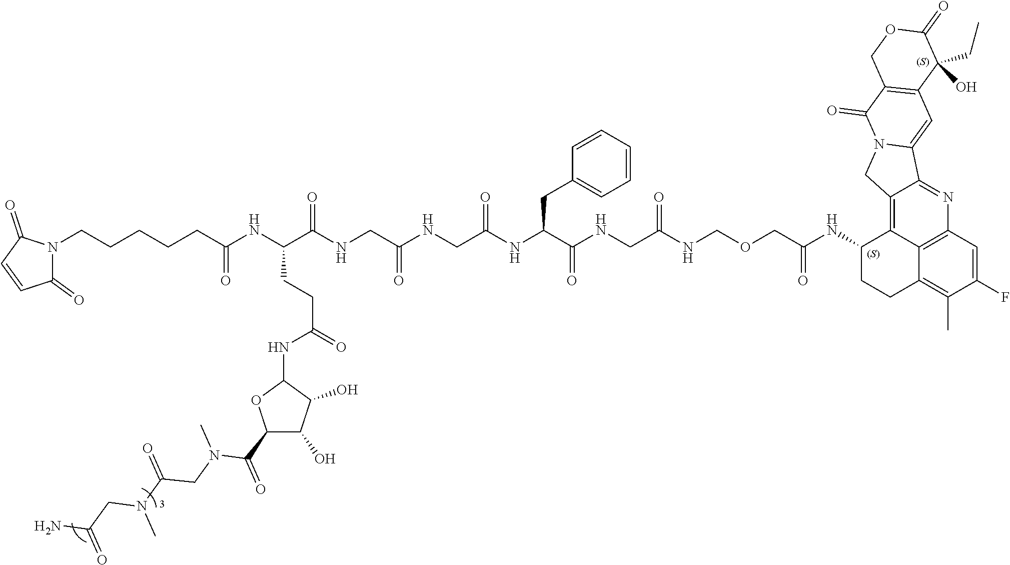 |
| | 34 |
| 35 | 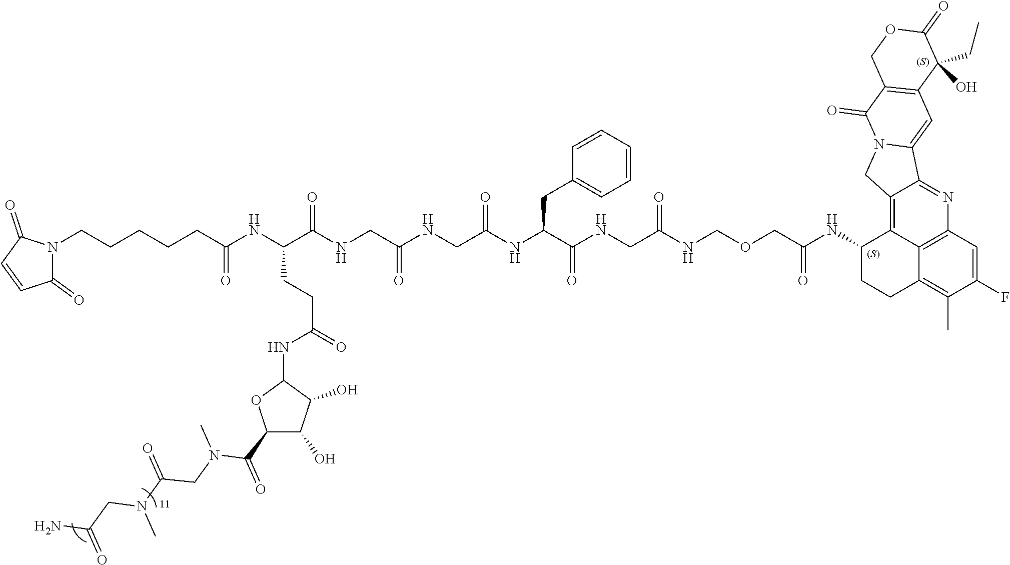 |
| | 35 |
| 36 | 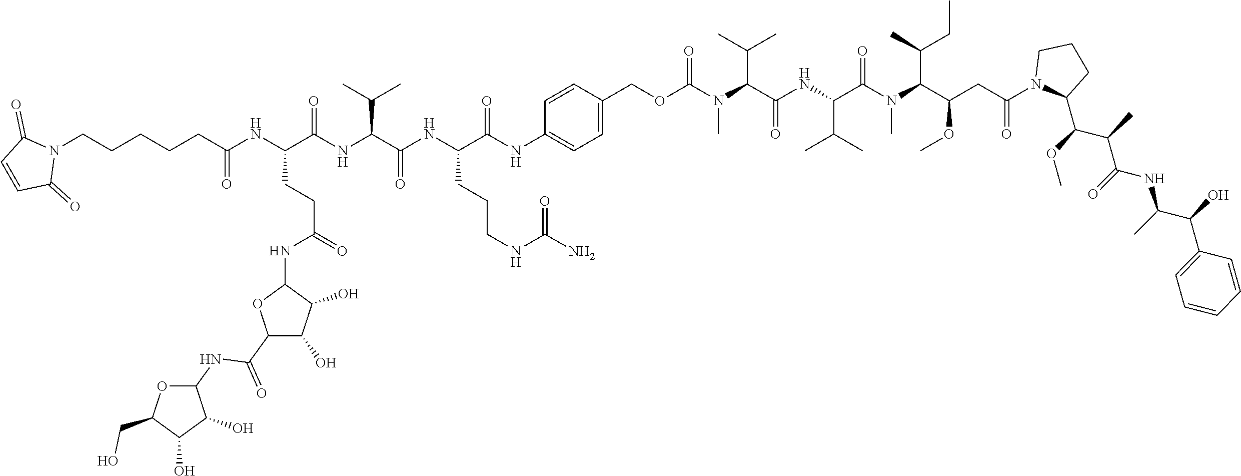 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)NC2OC(C(=O)NC3O[C@H](CO)[C@@H](O)[C@H]3O)[C@@H](O)[C@H]2O)NC(=O)CCCCCN2C(=O)C=CC2=O)C(C)C)cc1)C(C)C] |
| | 36 |
| 37 | 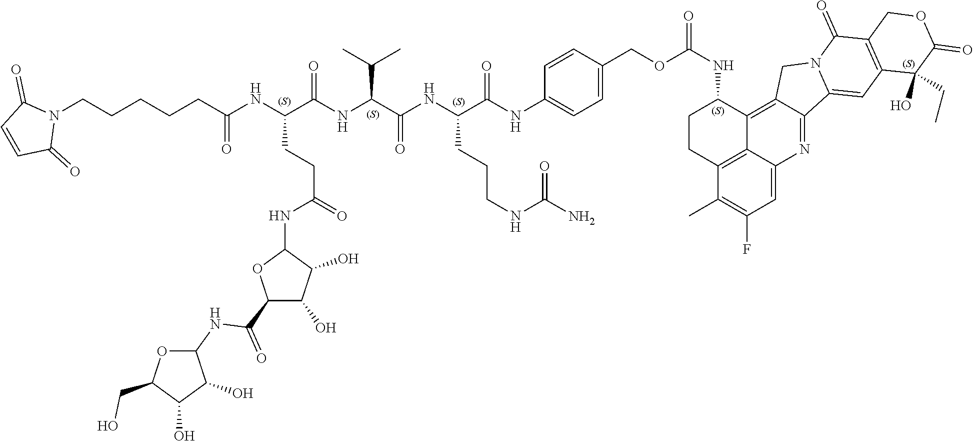 |
| | 37 |
| 38 | 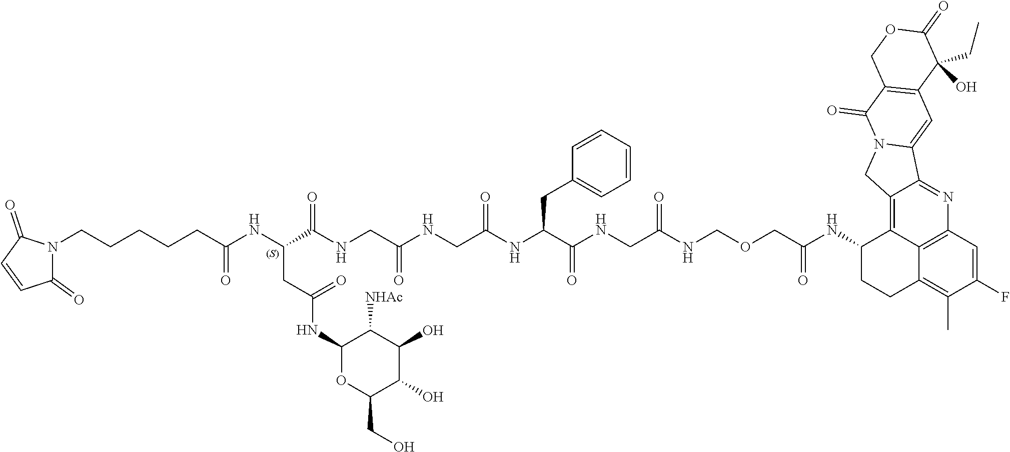 |
| | 38 |
| 39 | 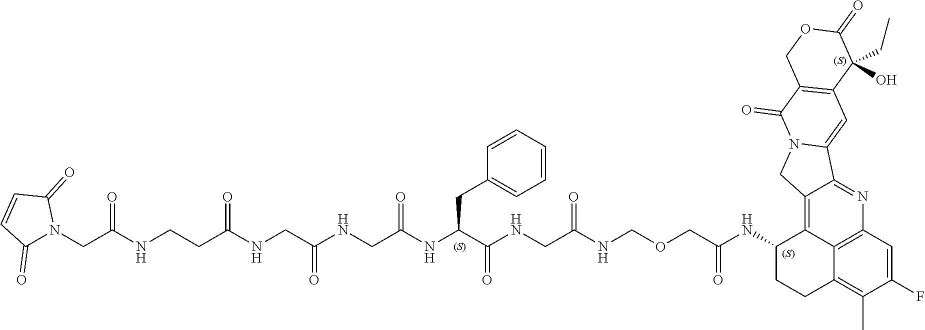 |
| | 39 |
| 40 | 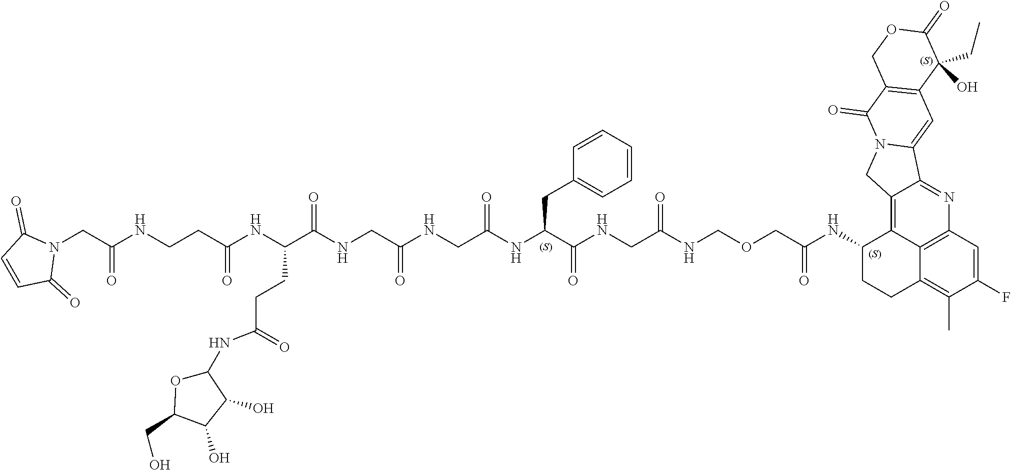 |
| | 40 |
| 41 | 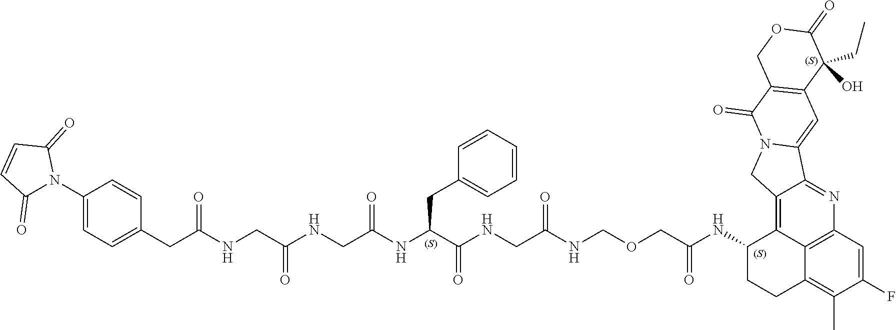 |
| | 41 |
| 42 | 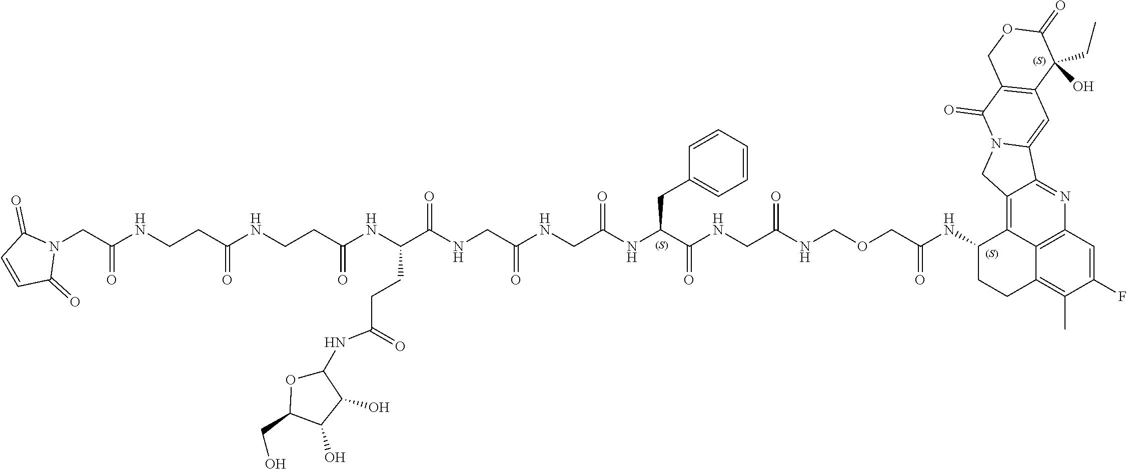 |
| | 42 |
| 43 | 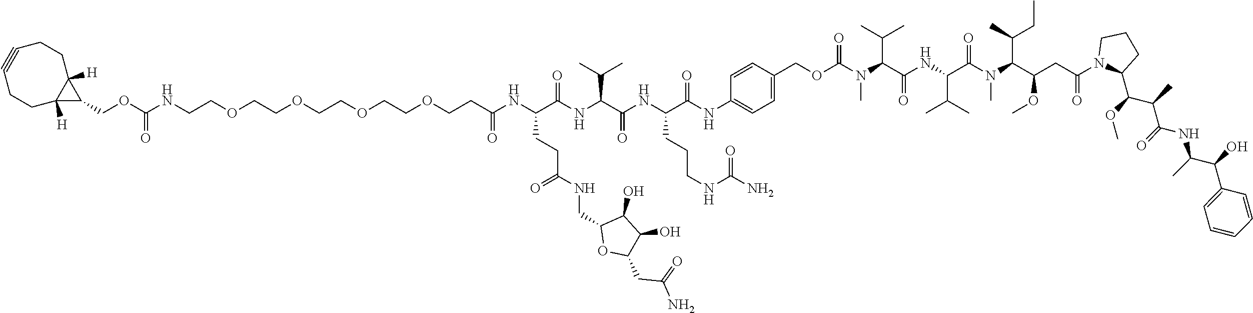 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)NC[C@H]2O[C@@H](CC(N)=O)[C@H](O)[C@@H]2O)NC(=O)CCOCCOCCOCCOCCNC(=O)OC[C@@H]2[C@@H]3CCC#CCC[C@@H]32)C(C)C)cc1)C(C)C] |
| | 43 |
| 44 | 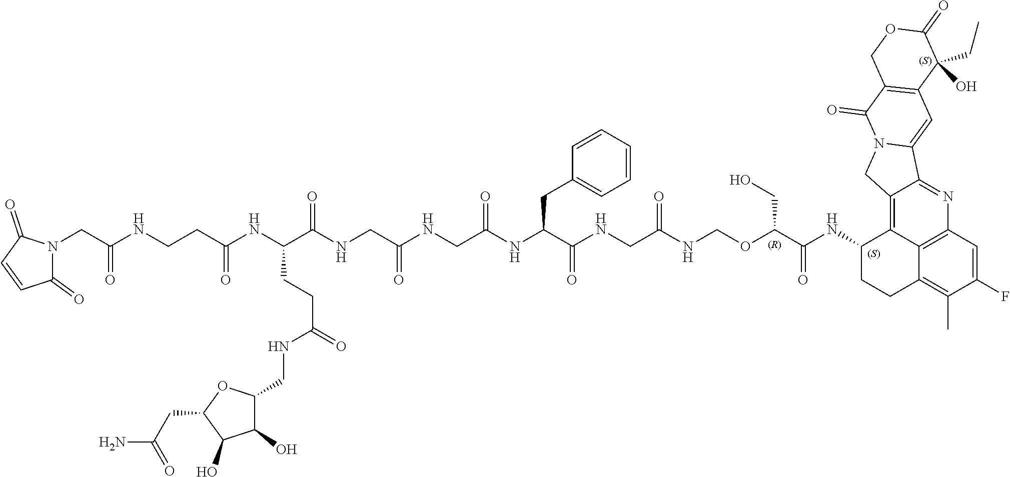 |
| | 44 |
| 45 | 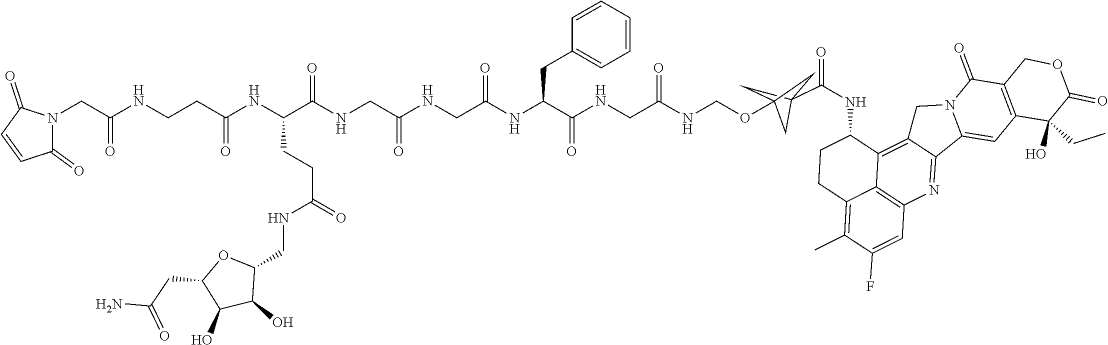 [SMILES: CC[C@@]1(O)C(=O)OCc2c1cc1n(c2=O)Cc2c-1nc1cc(F)c(C)c3c1c2[C@@H](NC(=O)C12CC(OCNC(=O)CNC(=O)[C@H](Cc4ccccc4)NC(=O)CNC(=O)CNC(=O)[C@H](CCC(=O)NC[C@H]4O[C@@H](CC(N)=O)[C@H](O)[C@@H]4O)NC(=O)CCNC(=O)CN4C(=O)C=CC4=O)(C1)C2)CC3] |
| | 45 |
| 46 | 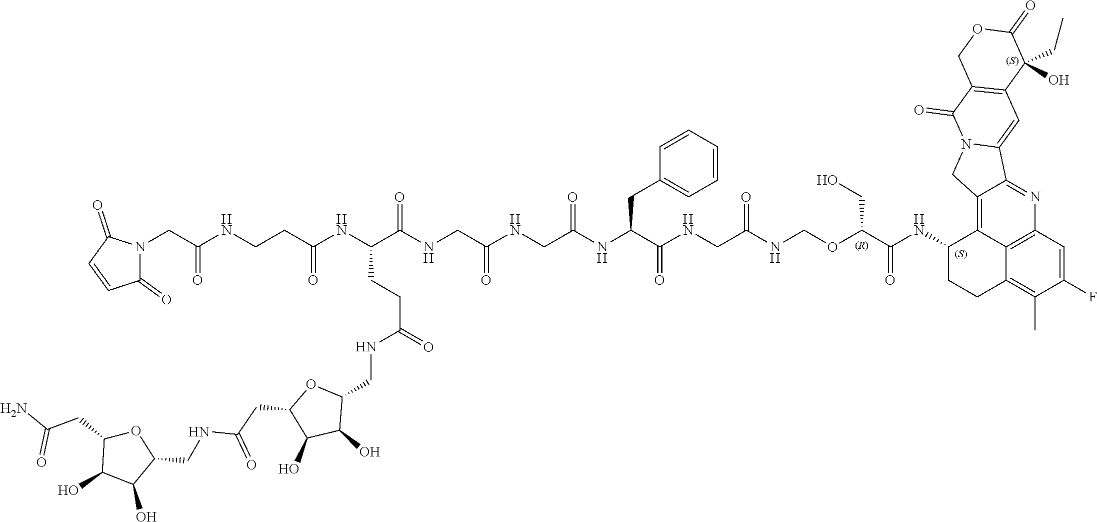 |
| | 46 |
| 47 | 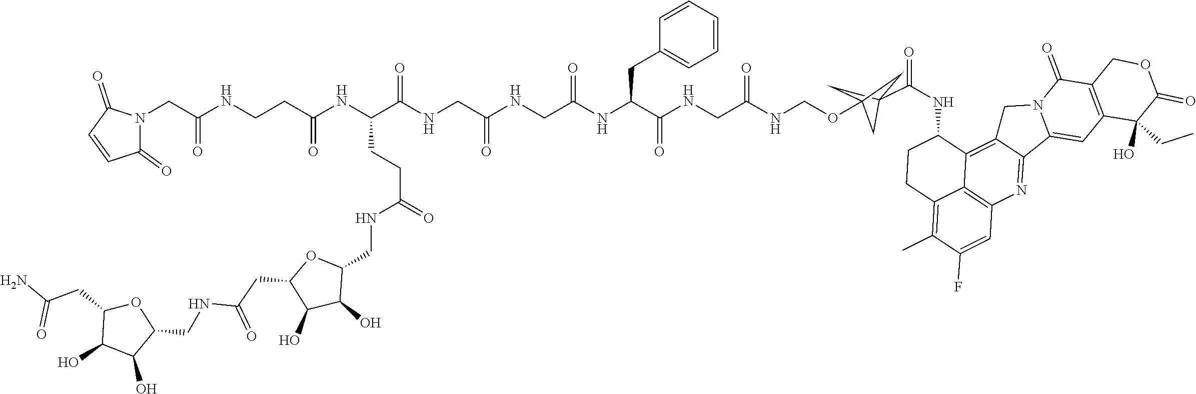 [SMILES: CC[C@@]1(O)C(=O)OCc2c1cc1n(c2=O)Cc2c-1nc1cc(F)c(C)c3c1c2[C@@H](NC(=O)C12CC(OCNC(=O)CNC(=O)[C@H](Cc4ccccc4)NC(=O)CNC(=O)CNC(=O)[C@H](CCC(=O)NC[C@H]4O[C@@H](CC(=O)NC[C@H]5O[C@@H](CC(N)=O)[C@H](O)[C@@H]5O)[C@H](O)[C@@H]4O)NC(=O)CCNC(=O)CN4C(=O)C=CC4=O)(C1)C2)CC3] |
| | 47 |
| 48 | 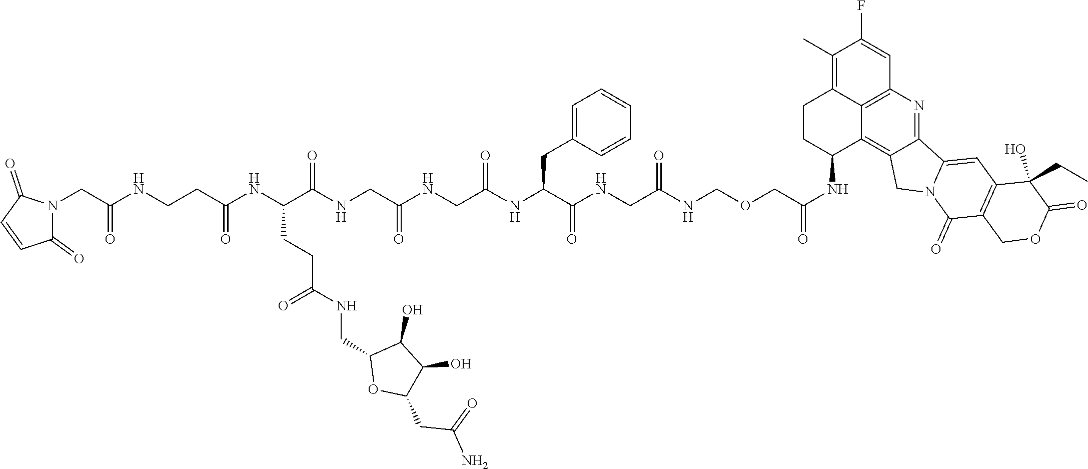 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)[C@H](CCC(=O)NC[C@H]1O[C@@H](CC(N)=O)[C@H](O)[C@@H]1O)NC(=O)CCNC(=O)CN1C(=O)C=CC1=O)CC3] |
| | 48 |
| 49 | 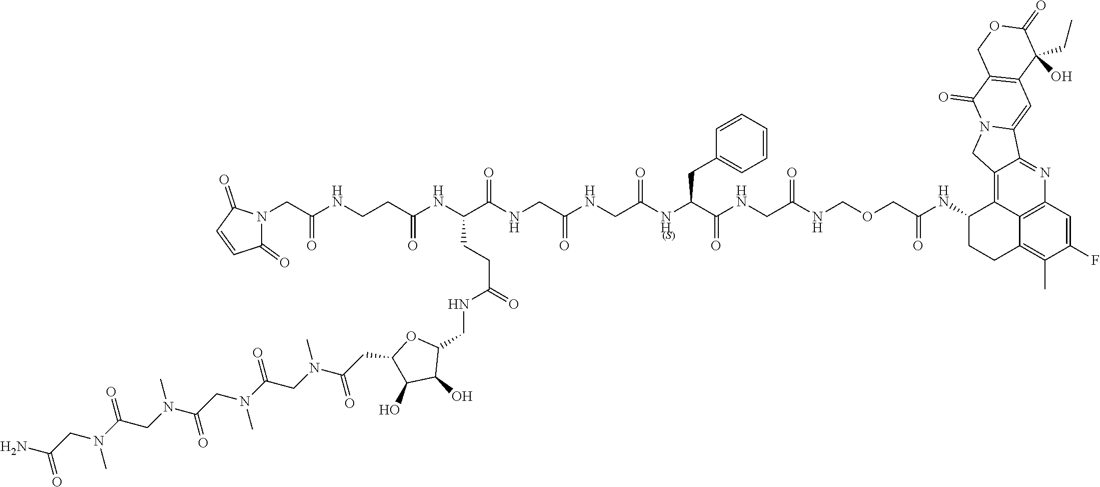 |
| | 49 |
| 50 | 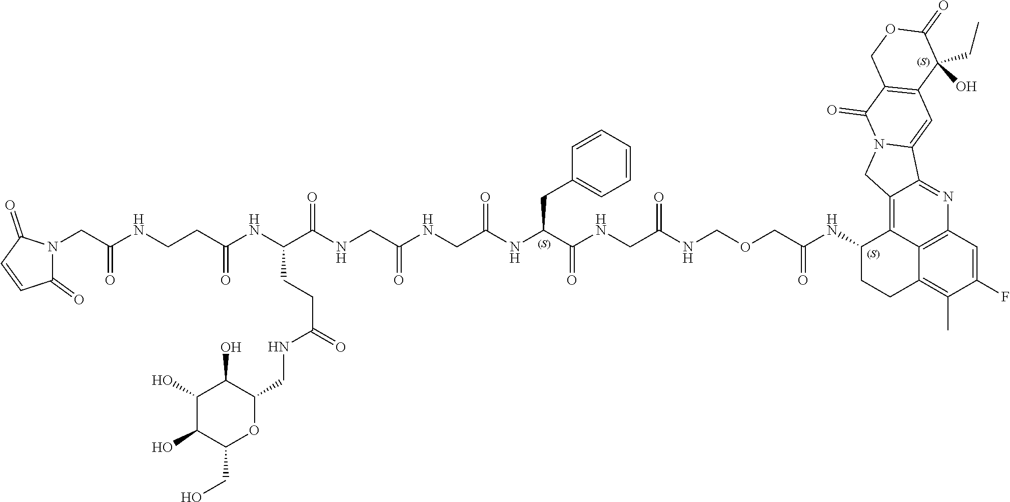 |
| | 50 |
| 51 | 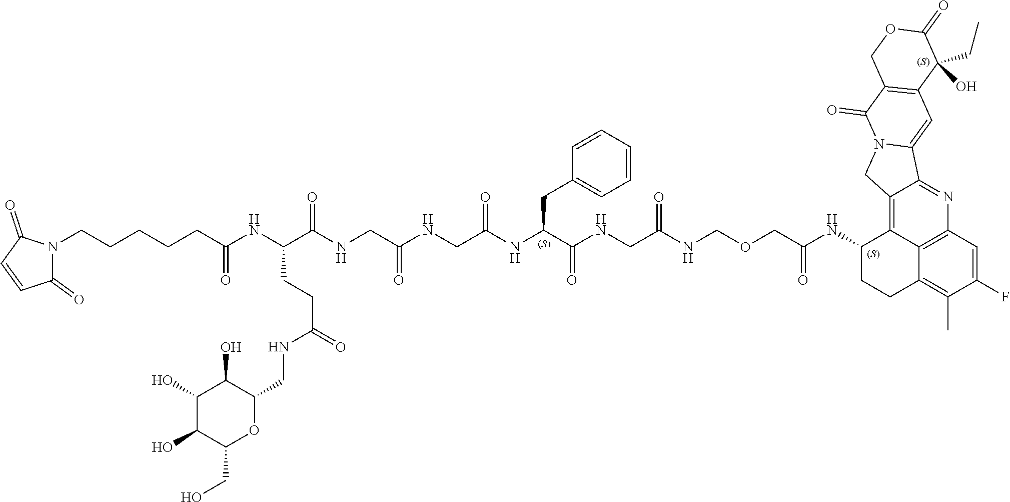 |
| | 51 |
| 52 | 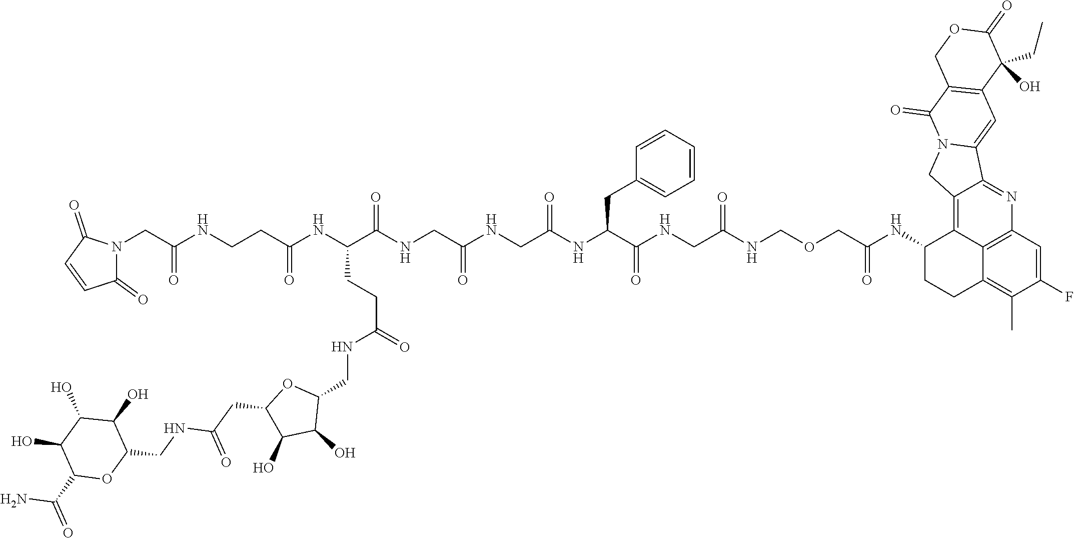 |
| | 52 |
| 53 | 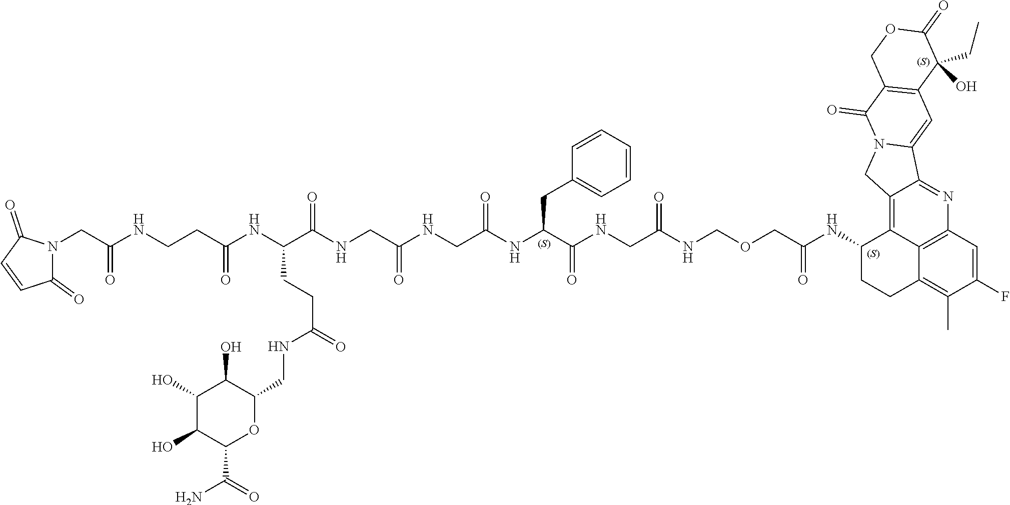 |
| | 53 |
| 54 | 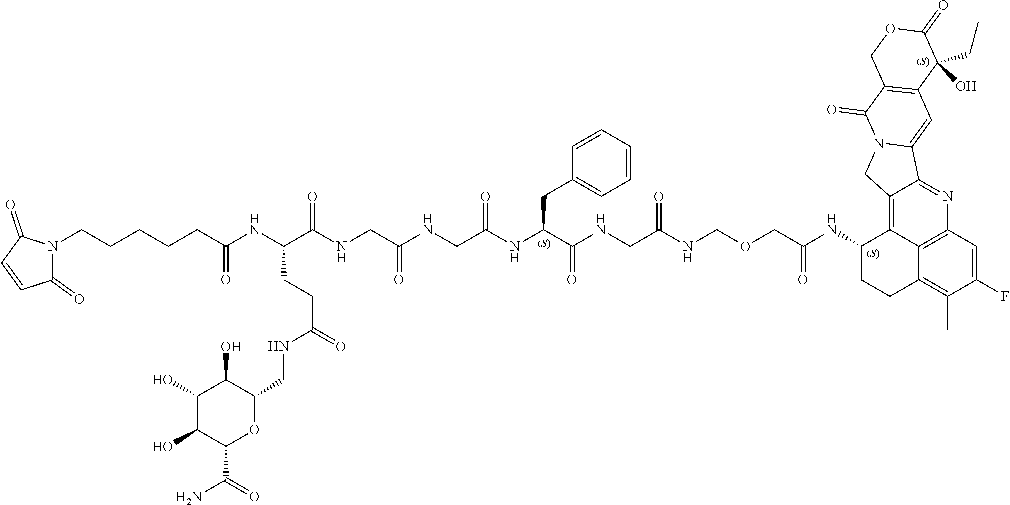 |
| | 54 |
| 55 | 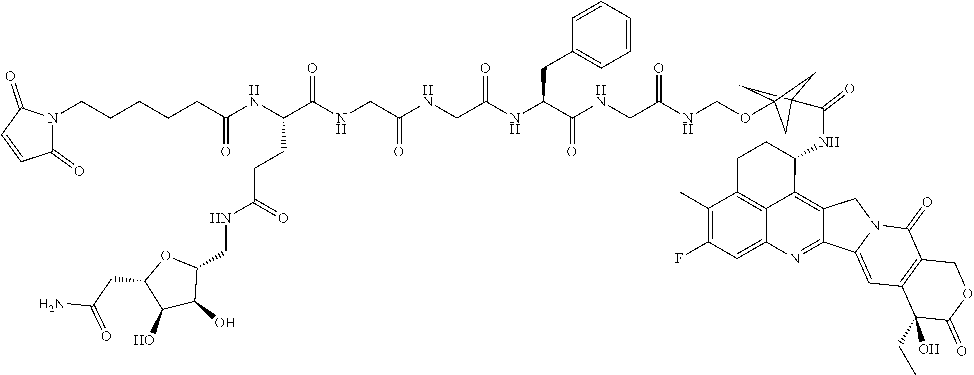 [SMILES: CC[C@@]1(O)C(=O)OCc2c1cc1n(c2=O)Cc2c-1nc1cc(F)c(C)c3c1c2[C@@H](NC(=O)C12CC(OCNC(=O)CNC(=O)[C@H](Cc4ccccc4)NC(=O)CNC(=O)CNC(=O)[C@H](CCC(=O)NC[C@H]4O[C@@H](CC(N)=O)[C@H](O)[C@@H]4O)NC(=O)CCCCCN4C(=O)C=CC4=O)(C1)C2)CC3] |
| | 55 |
| 56 | 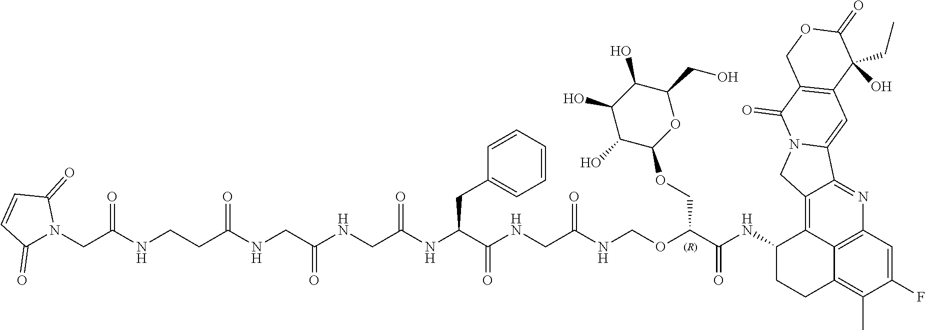 |
| | 56 |
| 57 | 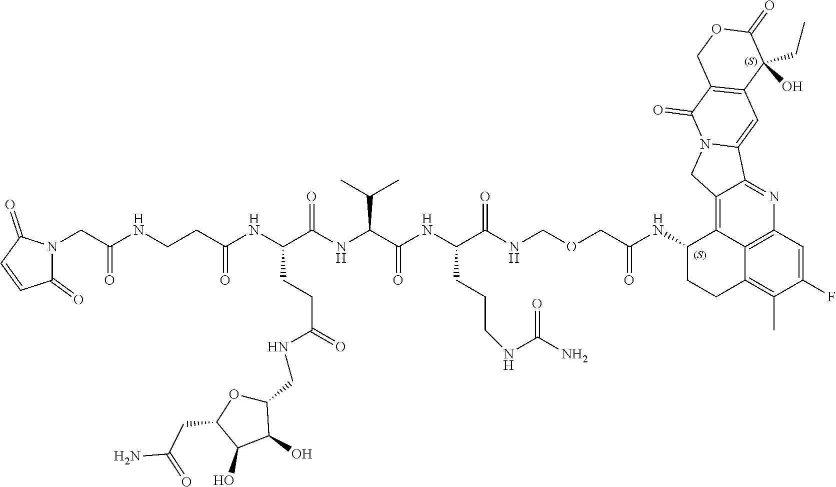 |
| | 57 |
| 58 | 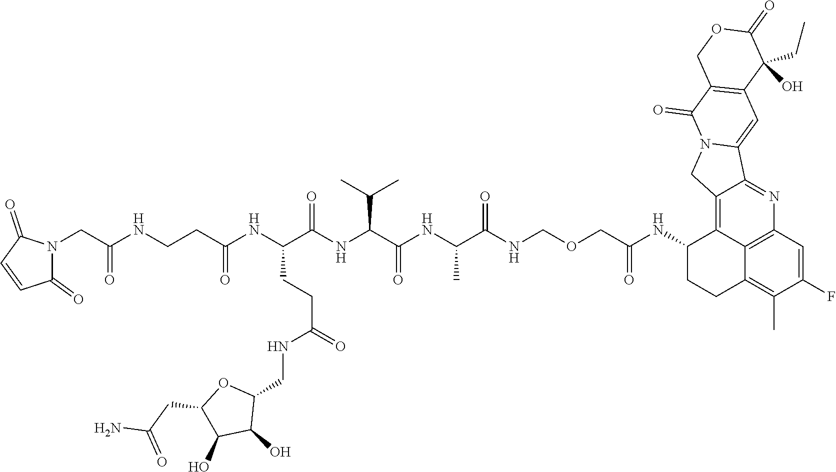 [SMILES: CC[C@@]1(O)C(=O)OCc2c1cc1n(c2=O)Cc2c-1nc1cc(F)c(C)c3c1c2[C@@H](NC(=O)COCNC(=O)[C@H](C)NC(=O)[C@@H](NC(=O)[C@H](CCC(=O)NC[C@H]1O[C@@H](CC(N)=O)[C@H](O)[C@@H]1O)NC(=O)CCNC(=O)CN1C(=O)C=CC1=O)C(C)C)CC3] |
| | 58 |
| 59 | 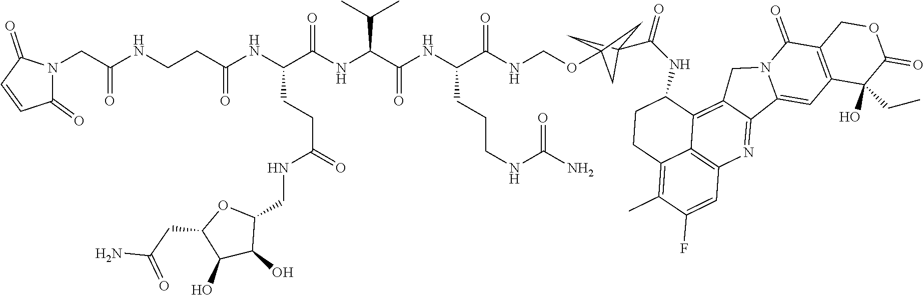 [SMILES: CC[C@@]1(O)C(=O)OCc2c1cc1n(c2=O)Cc2c-1nc1cc(F)c(C)c3c1c2[C@@H](NC(=O)C12CC(OCNC(=O)[C@H](CCCNC(N)=O)NC(=O)[C@@H](NC(=O)[C@H](CCC(=O)NC[C@H]4O[C@@H](CC(N)=O)[C@H](O)[C@@H]4O)NC(=O)CCNC(=O)CN4C(=O)C=CC4=O)C(C)C)(C1)C2)CC3] |
| | 59 |
| 60 | 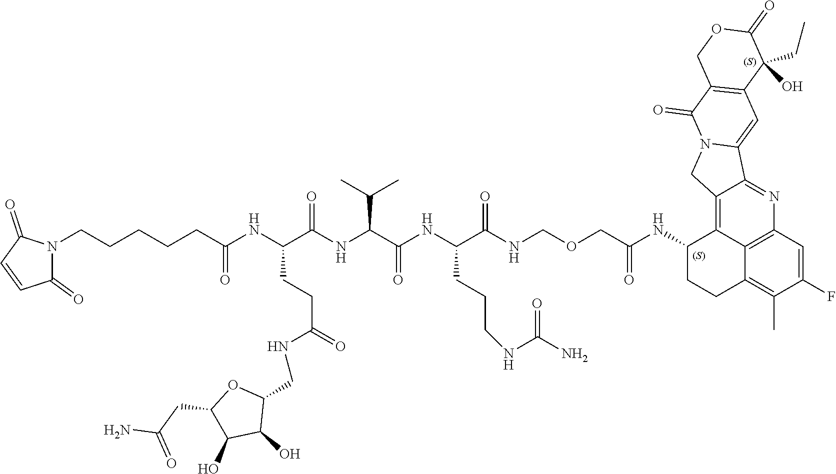 |
| | 60 |
| 61 | 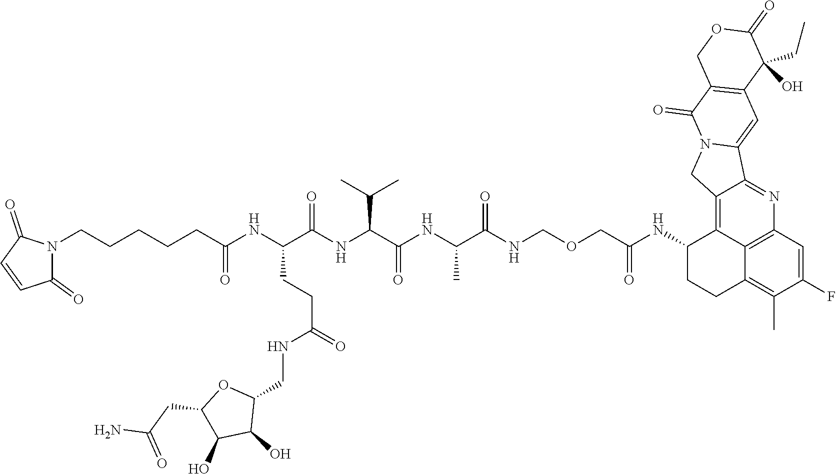 [SMILES: CC[C@@]1(O)C(=O)OCc2c1cc1n(c2=O)Cc2c-1nc1cc(F)c(C)c3c1c2[C@@H](NC(=O)COCNC(=O)[C@H](C)NC(=O)[C@@H](NC(=O)[C@H](CCC(=O)NC[C@H]1O[C@@H](CC(N)=O)[C@H](O)[C@@H]1O)NC(=O)CCCCCN1C(=O)C=CC1=O)C(C)C)CC3] |
| | 61 |
| 62 | 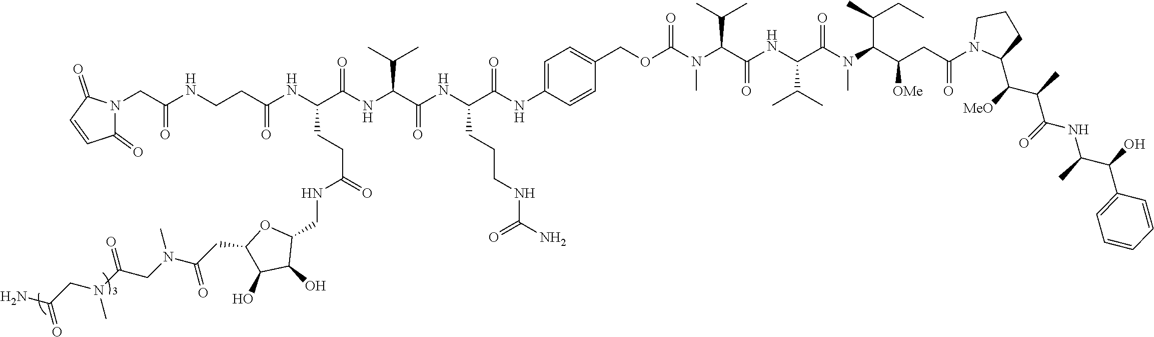 |
| | 62 |
| 63 | 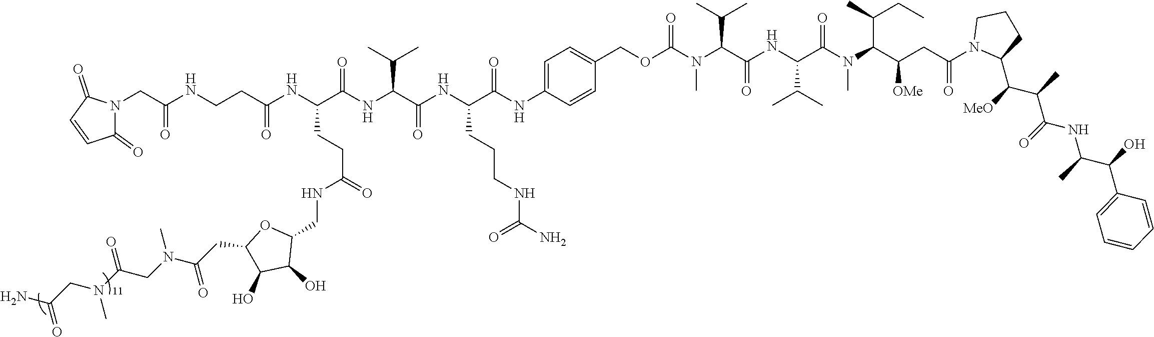 |
| | 63 |
| 64 | 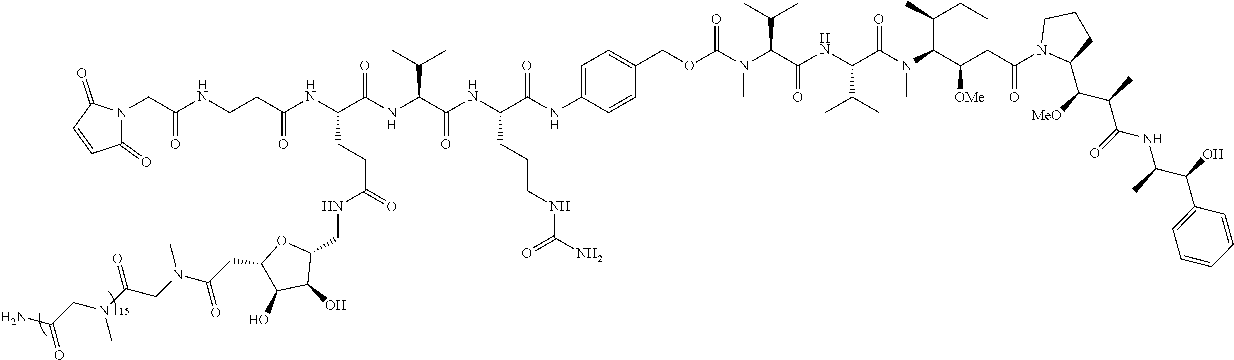 |
| | 64 |
| 65 | 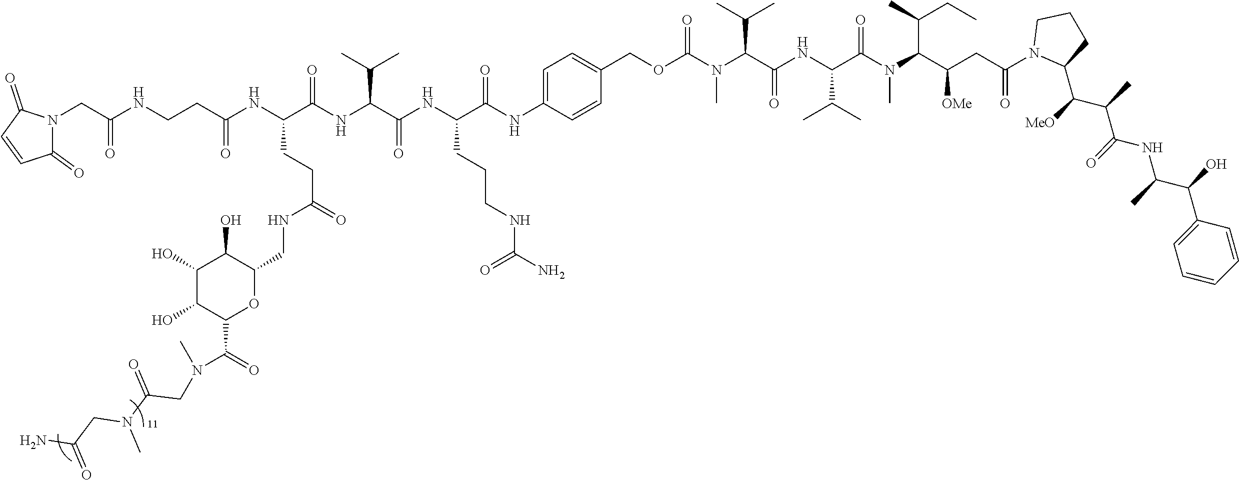 |
| | 65 |

TABLE 2
Capped linker with payload

| Examples | Antibody | Antibody drug conjugate |
|---|---|---|
| NAcCys- 13 | NAcCys- capped | 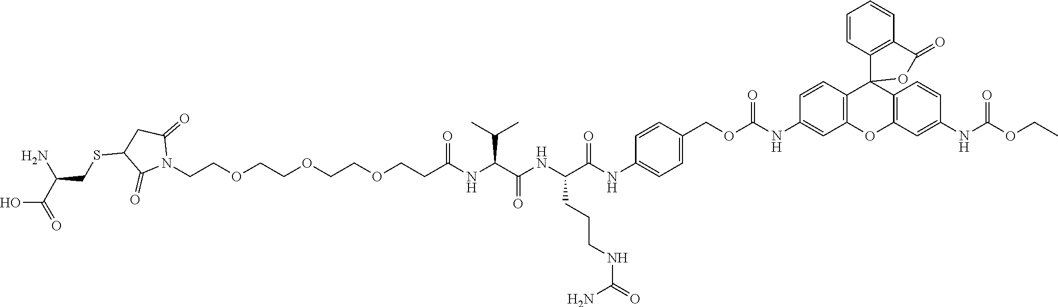 [SMILES: CCOC(=O)Nc1ccc2c(c1)Oc1cc(NC(=O)OCc3ccc(NC(=O)[C@H](CCCNC(N)=O)NC(=O)[C@@H](NC(=O)CCOCCOCCOCCN4C(=O)CC(SC[C@H](N)C(=O)O)C4=O)C(C)C)cc3)ccc1C21OC(=O)c2ccccc21] |
| NAcCys- 14 | NAcCys- capped | 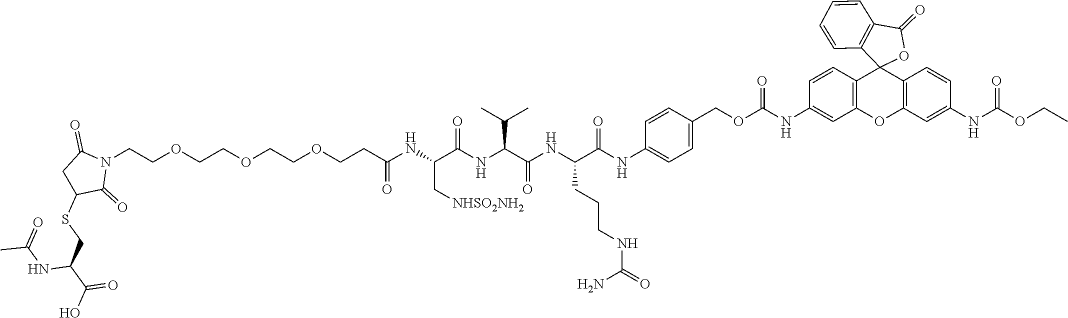 |
| NAcCys- 15 | NAcCys- capped | 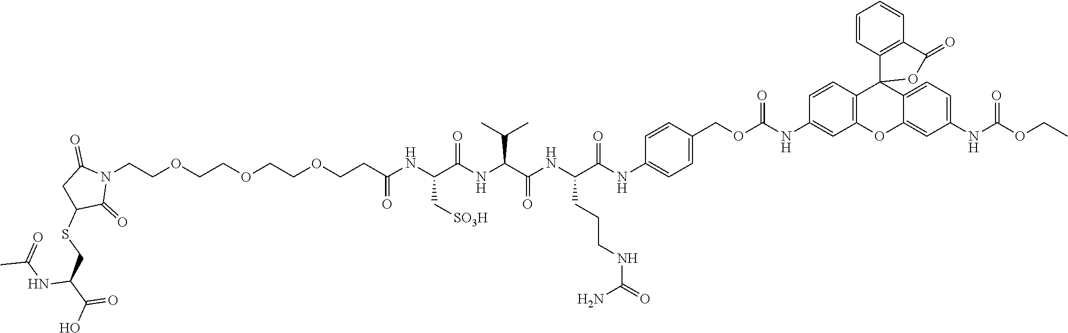 [SMILES: CCOC(=O)Nc1ccc2c(c1)Oc1cc(NC(=O)OCc3ccc(NC(=O)[C@H](CCCNC(N)=O)NC(=O)[C@@H](NC(=O)[C@H](CS(=O)(=O)O)NC(=O)CCOCCOCCOCCN4C(=O)CC(SC[C@H](NC(C)=O)C(=O)O)C4=O)C(C)C)cc3)ccc1C21OC(=O)c2ccccc21] |
| NAcCys- 16 | NAcCys- capped | 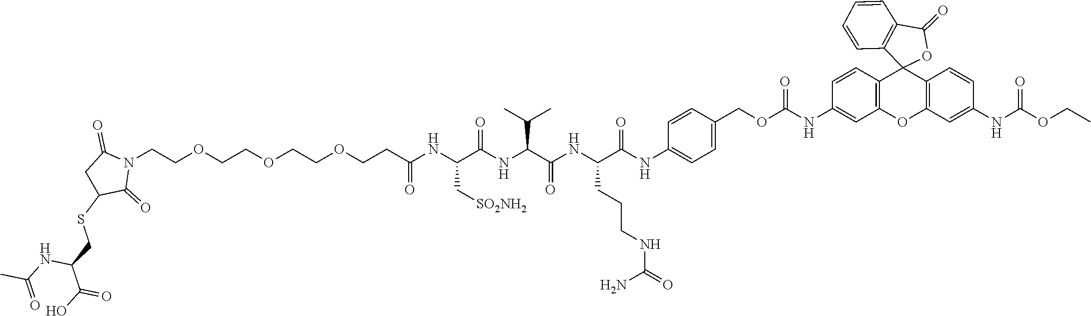 [SMILES: CCOC(=O)Nc1ccc2c(c1)Oc1cc(NC(=O)OCc3ccc(NC(=O)[C@H](CCCNC(N)=O)NC(=O)[C@@H](NC(=O)[C@H](CS(N)(=O)=O)NC(=O)CCOCCOCCOCCN4C(=O)CC(SC[C@H](NC(C)=O)C(=O)O)C4=O)C(C)C)cc3)ccc1C21OC(=O)c2ccccc21] |
| NAcCys- 17 | NAcCys- capped | 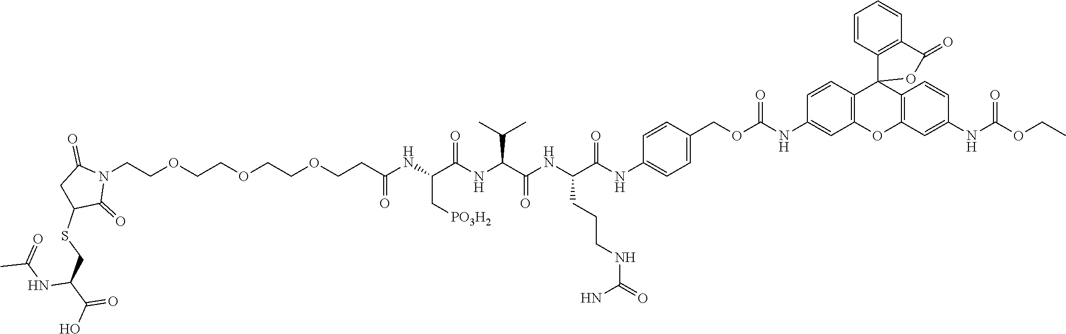 |

### Antibody Drug Conjugate Preparation

### DAR 4 Antibody Drug Conjugate Preparation

Antibody in conjugation buffer (with concentration 0.5-25 mg/mL, PBS buffer pH 6.0-8.5) was incubated under reduction temperature (0-40° C.) for 10 min and 2-10 eq. TECP solution (5 mM stock in PBS buffer) was added into the reaction mixture and left the reduction reaction for 1-8 hours at reduction temperature. Organic solvent (eg: DMSO, DMF, DMA, PG, acetonitrile, 5-25% v/v) and linker-payload stock (3-10 eq, 10 mM stock in organic solvent) were added stepwise after reduction mixture was cooled down to 0-25° C. Conjugation solution was left for 1-3 h at 0-25° C. and the reaction can be quenched with N-acetyl cysteine (1 mM stock). The solution was submitted to buffer exchange (spin desalting column, ultrafiltration, and dialysis) into storage buffer (for example: pH 5.5-6.5 histidine acetate buffer, with optional additive such as sucrose, trehalose, tween 20, 60, 80).

### DAR8 Antibody Drug Conjugate Preparation

Antibody in conjugation buffer (with concentration 0.5-25 mg/mL, PBS buffer pH 6.0-8.5) was incubated under reduction temperature (0-40° C.) for 10 min and 8-15 eq. TECP solution (5 mM stock in PBS buffer) was added into the reaction mixture and left the reduction reaction for 1-8 hours at reduction temperature. Organic solvent (eg: DMSO, DMF, DMA, PG, acetonitrile, 0-25% v/v) and linker-payload stock (10-25 eq, 10 mM stock in organic solvent) were added stepwise after reduction mixture was cooled down to 0-25° C. Conjugation solution was left for 1-3 h at 0-25° C. and the reaction can be quenched with N-acetyl Cysteine (1 mM stock). The solution was submitted to buffer exchange (spin desalting column, ultrafiltration, and dialysis) into storage buffer (for example: pH 5.5-6.5 Histidine acetate buffer, with optional additive such as sucrose, trehalose, tween 20, 60, 80).

### Organic Solvent Free Conjugation for DAR8 ADCs

Antibody in conjugation buffer (with concentration 0.5-25 mg/mL, PBS buffer pH 6.0-8.5) was incubated under reduction temperature (0-40° C.) for 10 min and 8-15 eq. TECP solution (5 mM stock in PBS buffer) was added into the reaction mixture and left the reduction reaction for 1-8 hours at reduction temperature. Linker-payload stock (10-25 eq, 10-200 mM conjugation buffer) was added after reduction mixture was cooled down to 0-25° C. Conjugation solution was left for 1-3 h at 0-25° C. and the reaction can be quenched with N-acetyl Cysteine (1 mM stock). The solution was submitted to buffer exchange (spin desalting column, ultrafiltration, and dialysis) into storage buffer (for example: pH 5.5-6.5 Histidine acetate buffer, with optional additive such as sucrose, trehalose, tween 20, 60, 80). ADC2-35, ADC2-57, ADC2-58, and ADC3-20 have been generated under organic free condition. It was surprising and unexpected to discover that the conjugation efficiency under organic free condition was remained similar as in organic solvent involved process. Organic free conjugation process is more cost saving than organic solvent involved process. It can reduce the cost of manufacture. The organic free conjugation process can be done in disposable single use bag. Furthermore, there is no clean validation and verification required for organic free conjugation processes.

### Maleimide Ring Opening Procedure

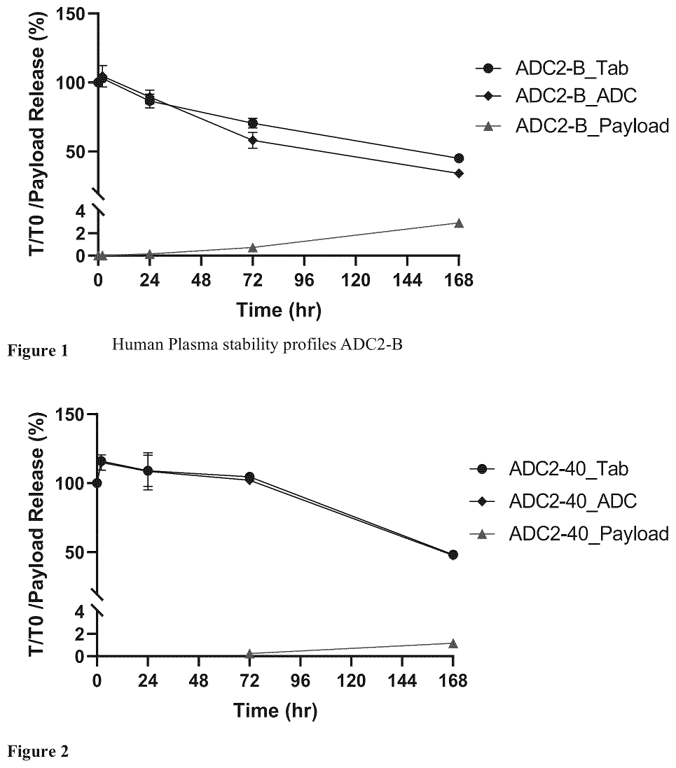

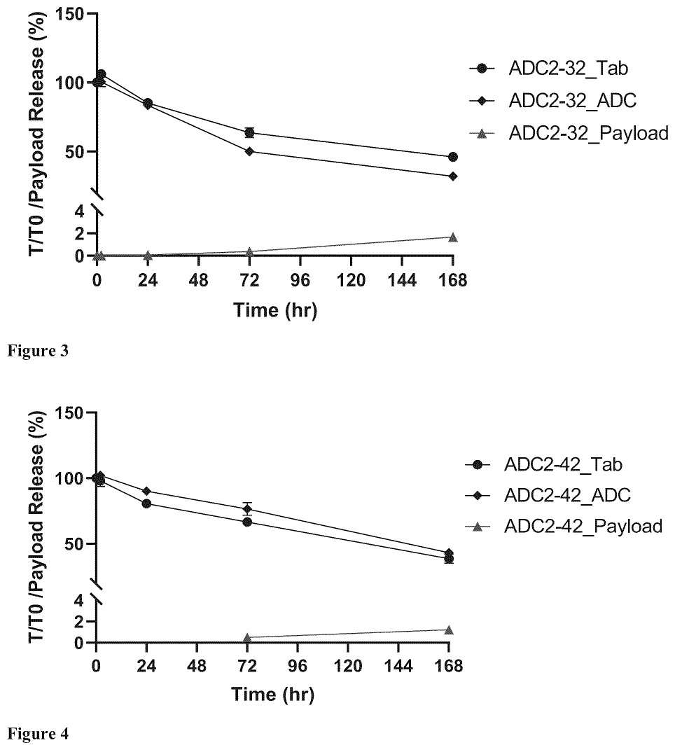

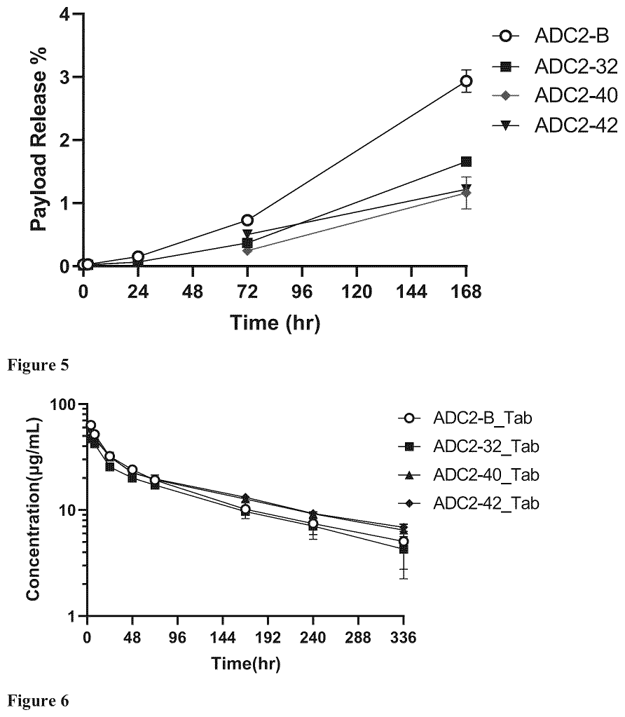

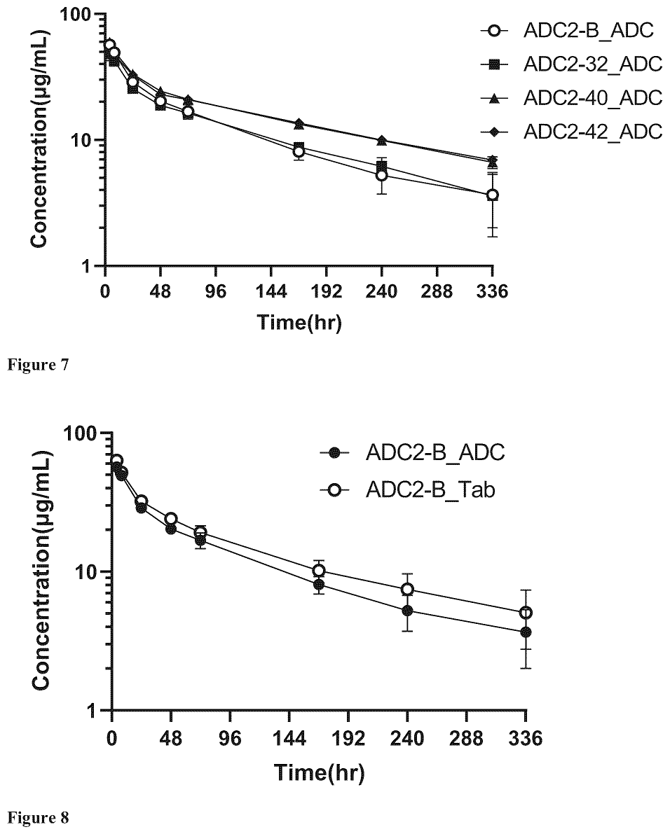

FIG. 1, FIG. 2, FIG. 3, and FIG. 4 illustrate that the human plasma stability of the ring-open ADCs is consistent with those of the corresponding antibodies. FIG. 5 demonstrates that the ADCs with their rings opened have their free payload release lower than the ADCs with no ring opening treatment in human plasma. FIG. 7 illustrates that the ADCs with ring-opening clear slower than the ADCs without ring-opening in rats. Maleimide ring is opened by the following procedures.

After the conjugation step, the ADC was undergone buffer exchange into ring opening buffer (pH 8.0˜9.0, borate or tris buffer) and the solution was left at 22 or 37 C for 5-48 h. Ring opening process was monitored via reduced LCMS. Once the ring opening is completed, the resulting ADCs were buffer exchanged into basic Tris pH 8.0-8.5 buffer or acidic histidine-acetate pH 5.0-6.5 buffer via dialysis.

### Glycan Engineering Site Specific Conjugation

Both glycan engineering steps (trimming and glycan transfer) were followed the literature reported procedure (2015 Bioconju. Chem. doi.org/10.1021/acs.bioconjchem.5b00224; 2018 Antibodies doi.org/10.3390/antib7010012). The [3+2] click chemistry conjugation step was applied via method reported in 2015 Bioconju. Chem. doi.org/10.1021/acs.bioconjchem.5b00224.

### ADC Characterization

ADC examples in this invention were prepared by following above procedures with DAR 2, 4, and 8 profiles. All ADCs in this invention were characterized via following analytical methods.

### Drug to Antibody Ratio (DAR) Determination

### LCMS Method

LC-MS analysis was carried out under the following measurement conditions:
LC-MS system: Vanquish Flex UHPLC and Orbitrap Exploris 240 Mass Spectrometer
Column: MAbPac™ RP, 2.1*50 mm, 4 μm, 1,500 Å, Thermo Scientific™
Column temperature: 80° C.
Mobile phase A: 0.1% formic acid (FA) aqueous solution
Mobile phase B: Acetonitrile solution containing 0.1% formic acid (FA)
Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)
Injected sample amount: 1 g
MS parameters: Intact and denaturing MS data were acquired in HMR mode at setting of R=15k and deconvolved using the ReSpect™ algorithm and Sliding Window integration in Thermo Scientific™ BioPharma Finder™ 4.0 software.

### HIC Method

HPLC analysis was carried out under the following measurement conditions:
HPLC system: Waters ACQUITY ARC HPLC System
Detector: measurement wavelegth: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM Phosphate buffer, pH 7.0
Mobile phase B: 50 mM Phosphate buffer, 25% (V/V) Isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 g

### ADC Purity

### SEC Method

HPLC analysis was carried out under the following measurement conditions:
HPLC system: Waters H-Class UPLC System
Detector: measurement wavelegth: 280 nm
Column: ACQUITY UPLC BEH200 SEC 1.7 um 4.6×150 mm, Waters
Column temperature: room temperature
Mobile phase A: 200 mM Phosphate buffer, 250 mM potassium chloride, 15% isopropyl alcohol, PH 7.0
Gradient program: under 10 min isocratic elutions with the flow rate of 0.3 mL/min
Injected sample amount: 20 g

### ADC Hydrophobicity Evaluation

### HIC Method

HPLC analysis was carried out under the following measurement conditions:
HPLC system: Waters ACQUITY ARC HPLC System
Detector: measurement wavelength: 280 nm
Column: MABPac HIC-10, 5 μm, 4.6×10 mm (Thermo)
Column temperature: 25° C.
Mobile phase A: 1.5 M ammonium sulfate, 50 mM sodium phosphate, pH 7.0
Mobile phase B: 50 mM sodium phosphate, pH 7.0
Gradient program: 20% B—20% B (0 min-1 min), 0O % B-0% B (1 min-35 min), 20% B-20% B (35 min-40 min)
Flow rate: 0.5 mL/min
Sample preparation: The sample was diluted with initial mobile phase to 0.5 mg/mL.

### SEC Purity

HPLC analysis was carried out under the following measurement conditions:
HPLC system: Waters H-Class UPLC System
Detector: measurement wavelength: 280 nm
Column: ACQUITY UPLC BEH200 SEC 1.7 um 4.6×150 mm, Waters
Column temperature: room temperature
Mobile phase A: 200 mM Phosphate buffer, 250 mM potassium chloride, 15% isopropyl alcohol, PH 7.0
Gradient program: under 10 min isocratic elution with the flow rate of 0.3 mL/min
Injected sample amount: 20 g

TABLE 3
ADC Examples

| | | | HIC |
|---|---|---|---|
| | | | DAR8 |
| | | | RT |
| Examples | Antibody | Antibody drug conjugate | (min) |
| ADC1-2 | Cofetu- zumab | 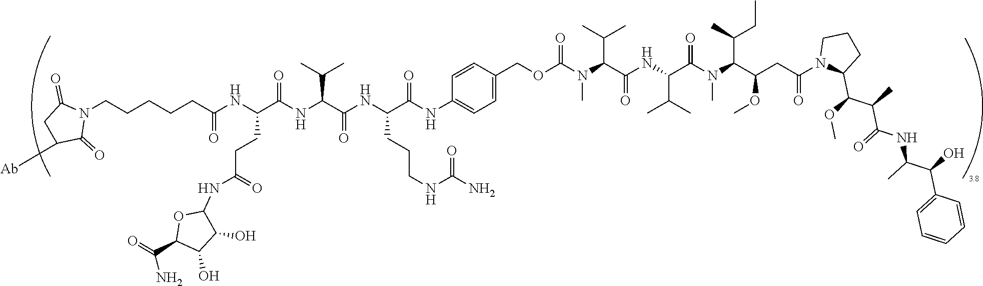 | 14.67* |
| ADC1-4 | Cofetu- zumab | 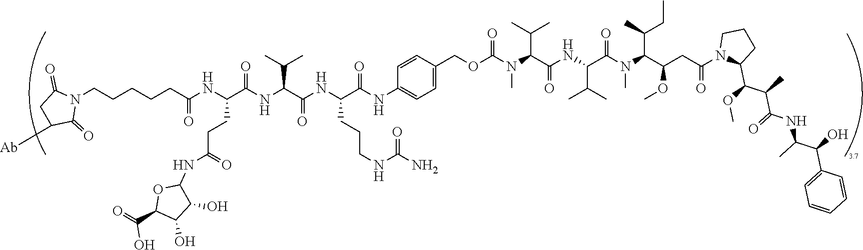 | 14.50* |
| ADC1-11 | Cofetu- zumab | 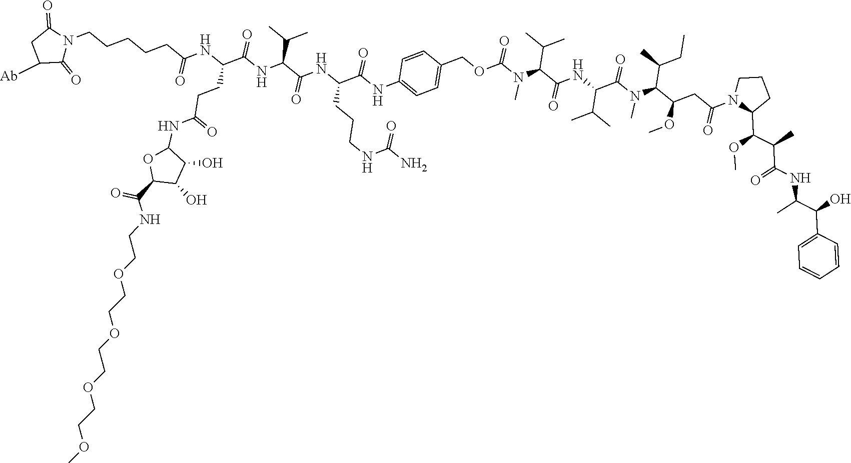 | 14.77* |
| ADC2-2 | trastu- zumab | 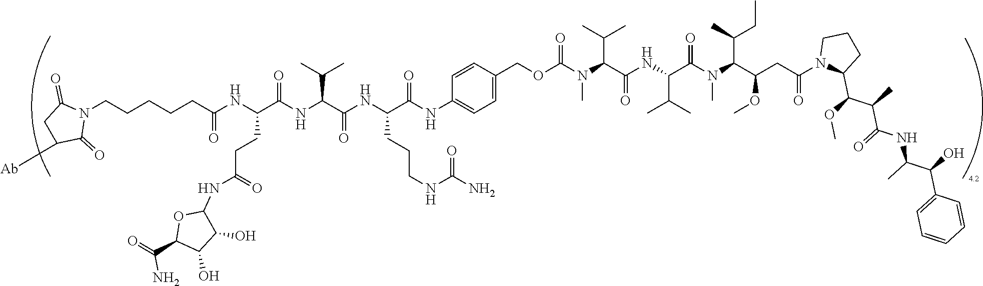 | 14.7* |
| ADC2-4 | trastu- zumab | 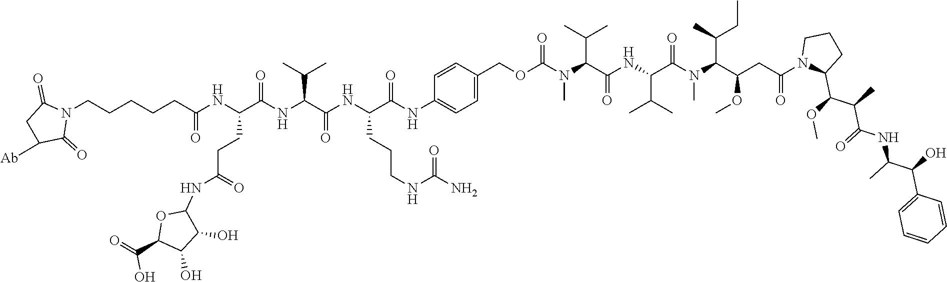 | 14.4* |
| ADC2-6-2 | trastu- zumab | 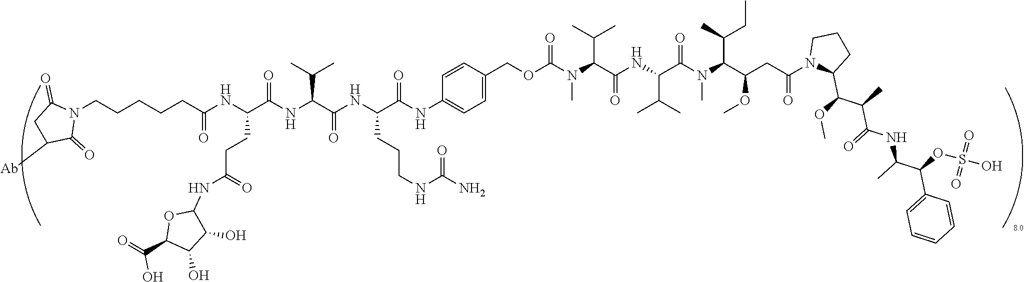 | 13.7* |
| ADC2-7-2 | trastu- zumab | 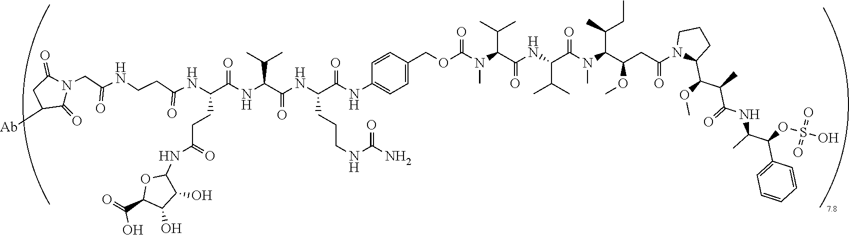 | 13.7* |
| ADC2-9-1 | trastu- zumab | 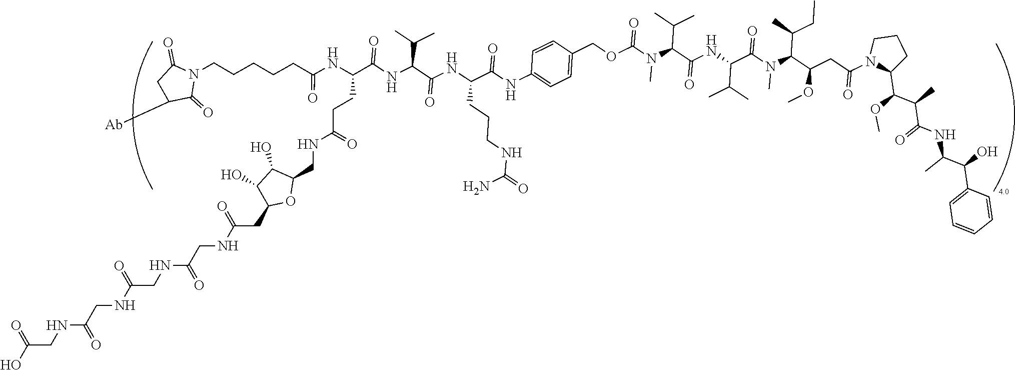 | 13.8* |
| ADC2-9-2 | trastu- zumab | 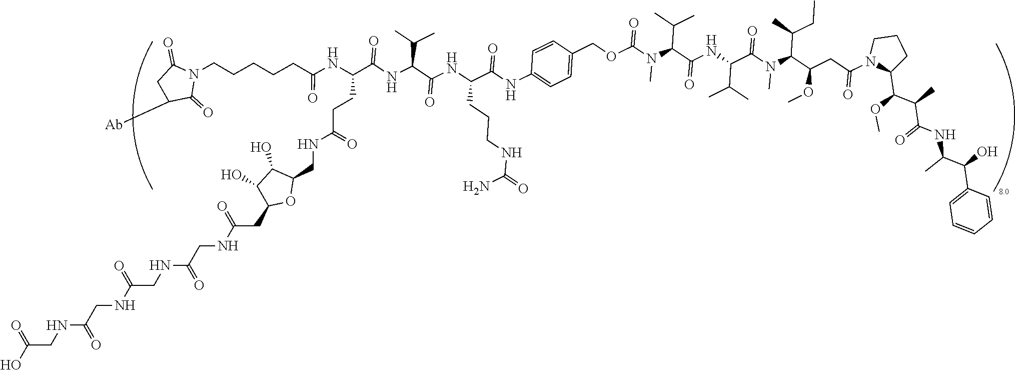 | 13.8* |
| ADC2-10 | trastu- zumab | 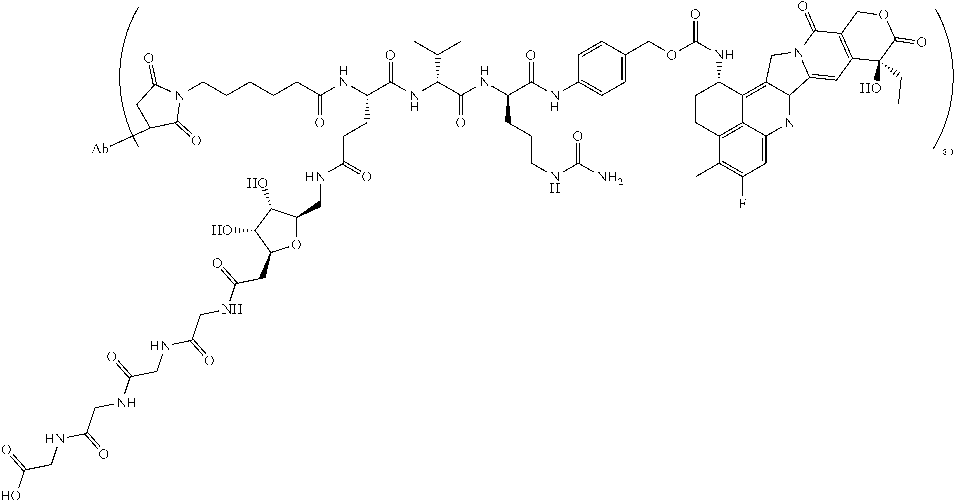 | 14.5* |
| ADC2-11 | trastu- zumab | 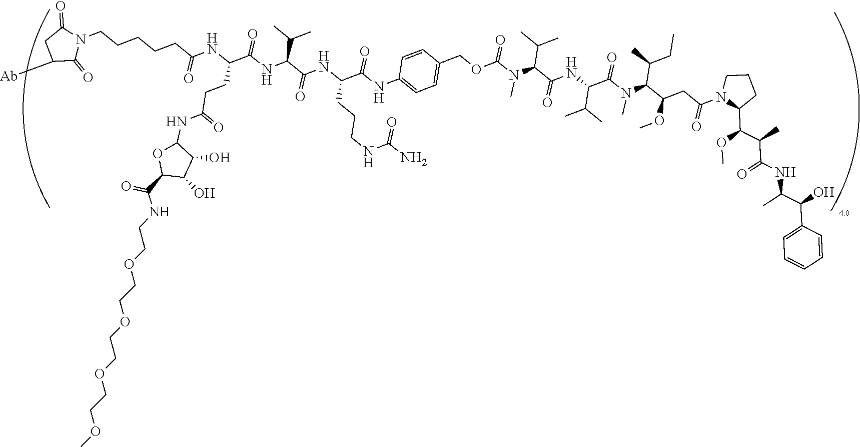 | 14.8* |
| ADC2-22 | trastu- zumab | 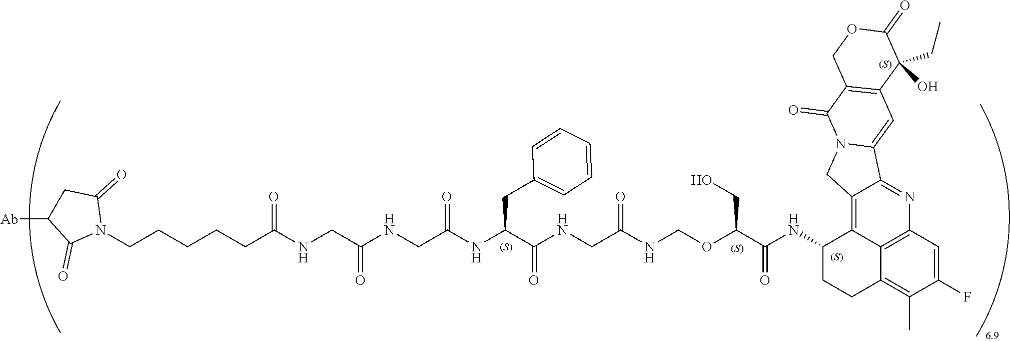 | NA |
| ADC2-23 | trastu- zumab | 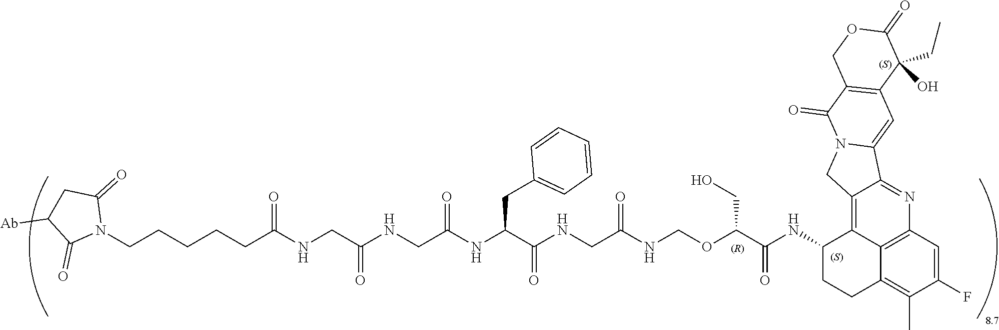 | 18.799 |
| ADC2-24 | trastu- zumab | 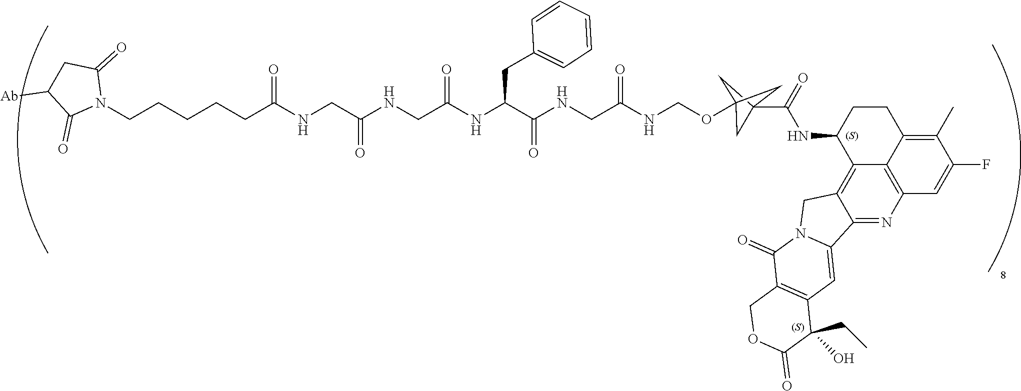 | 21.122 |
| ADC2-25 | trastu - zumab | 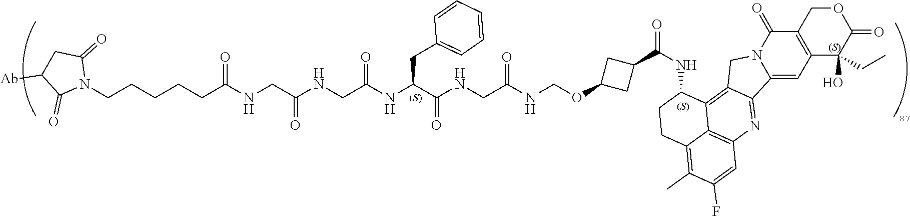 | 21.536 |
| ADC2-26 | trastu- zumab | 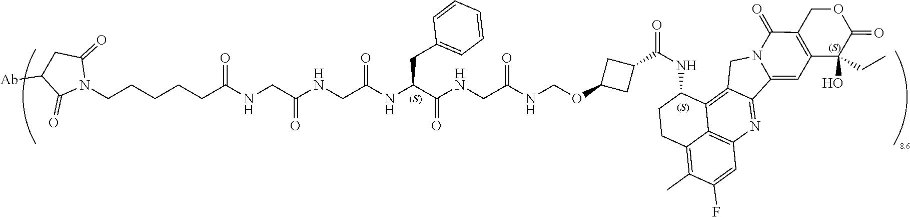 | 21.627 |
| ADC2-27 | trastu- zumab | 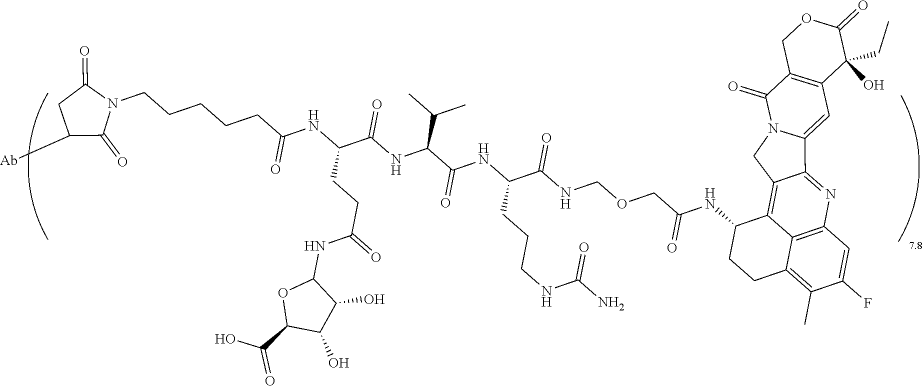 | 16.162 |
| ADC2-29 | trastu - zumab | 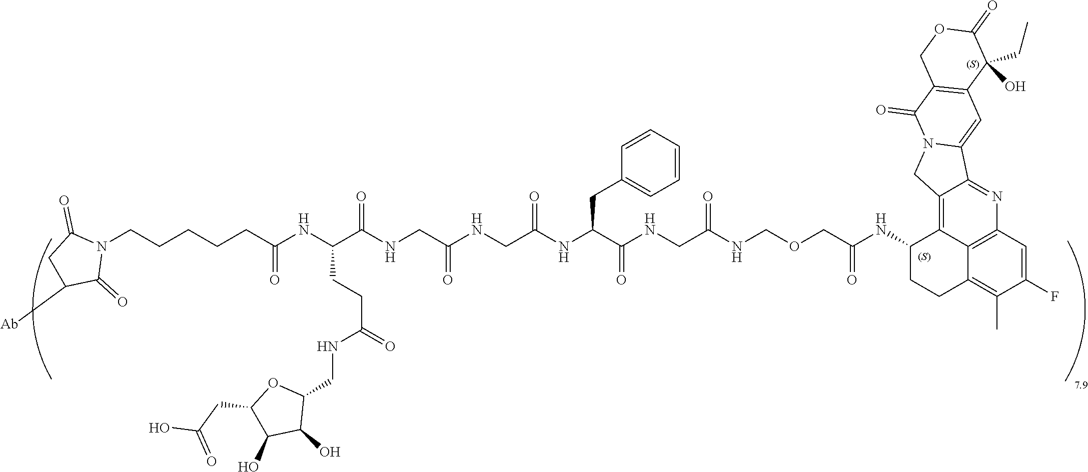 | 17.576 |
| ADC2-30 | trastu - zumab | 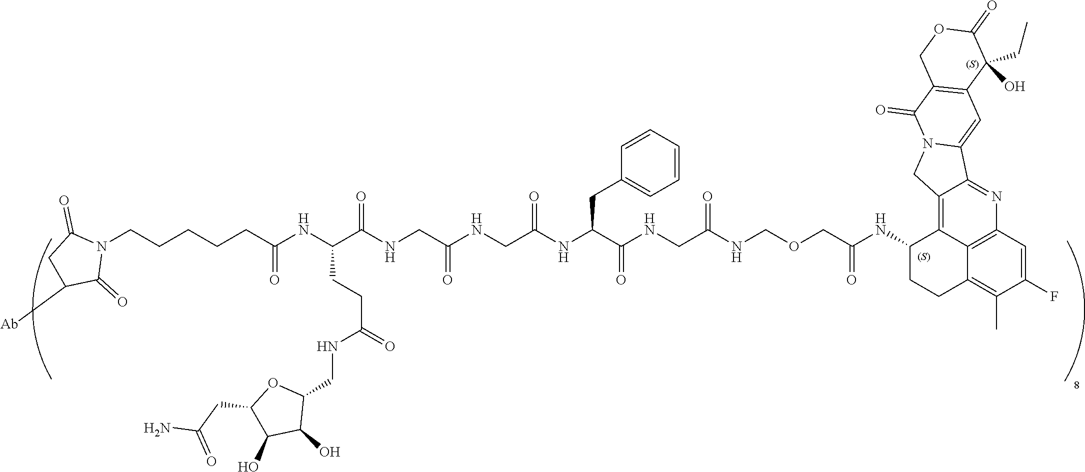 | 18.758 |
| ADC2-31 | trastu- zumab | 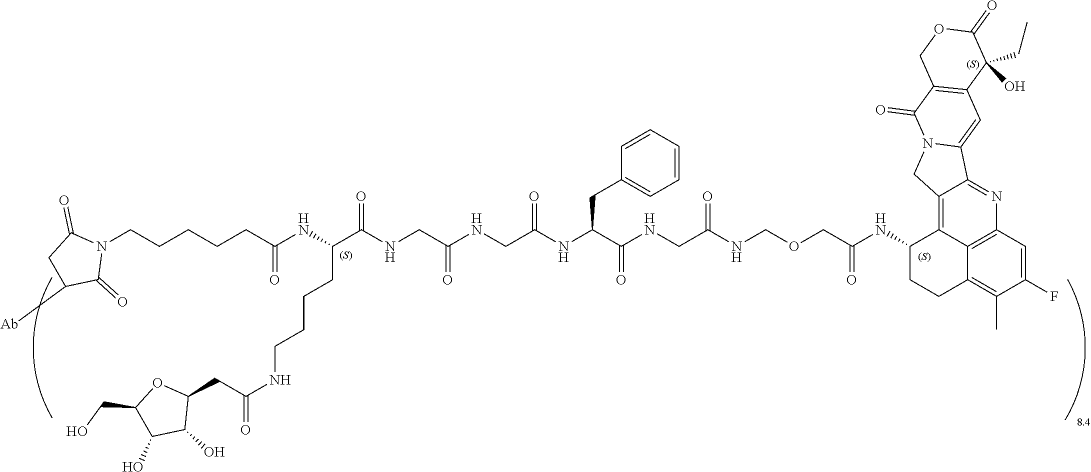 | 19.189 |
| ADC2-32 | trastu- zumab | 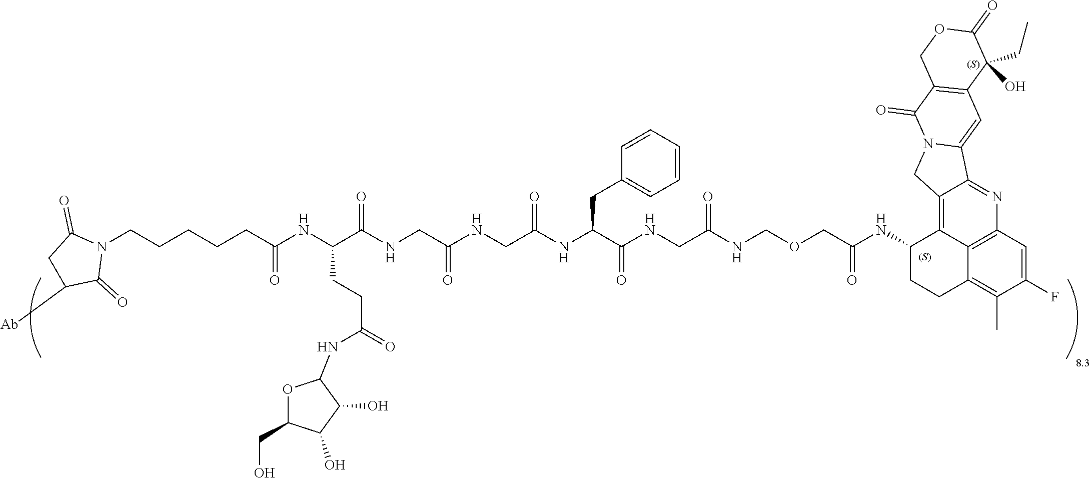 | 18.657 |
| ADC2-33 | trastu- zumab | 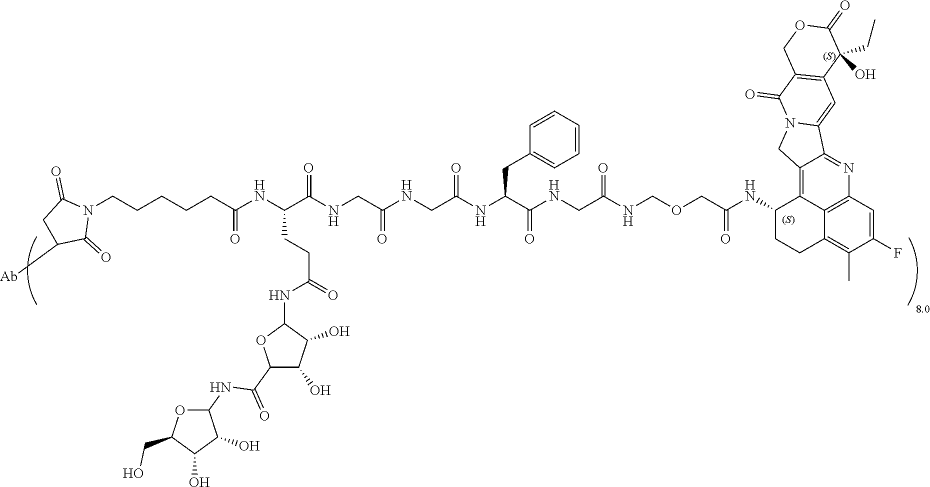 | 17.747 |
| ADC2-34 | trastu- zumab | 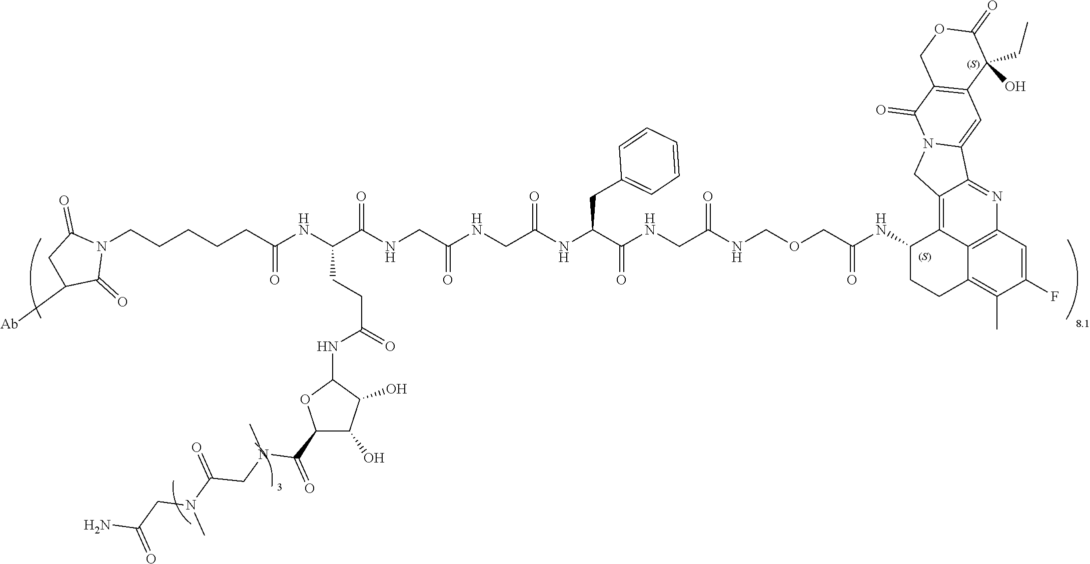 | 17.683 |
| ADC2-35 | trastu- zumab | 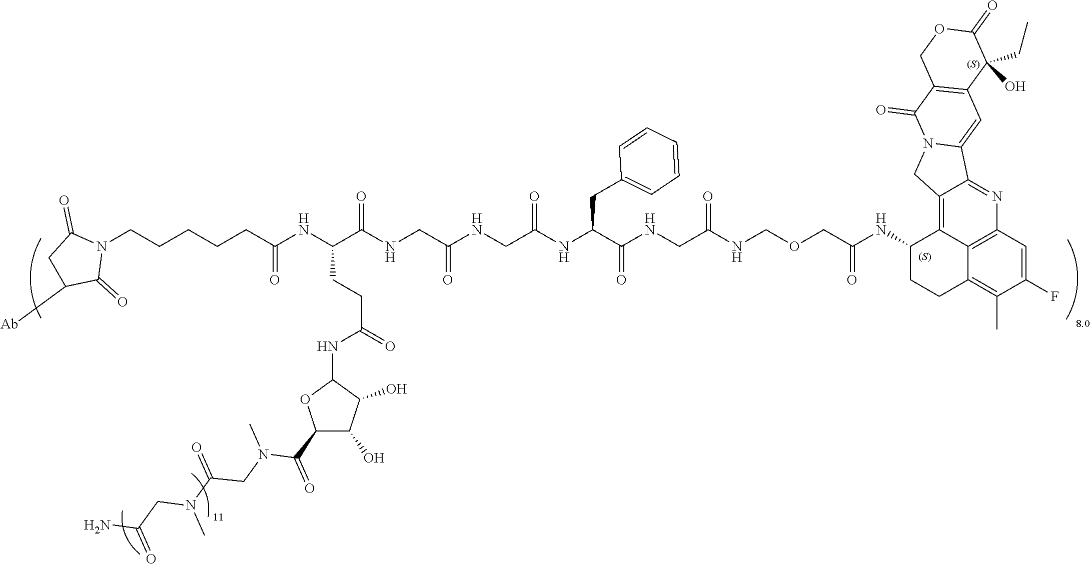 | 17.146 |
| ADC2-36-1 | trastu- zumab | 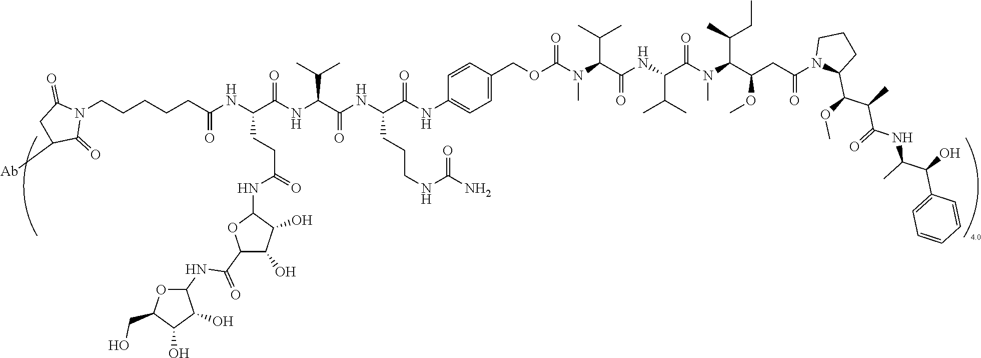 | 14.6* |
| ADC2-36-2 | trastu- zumab | 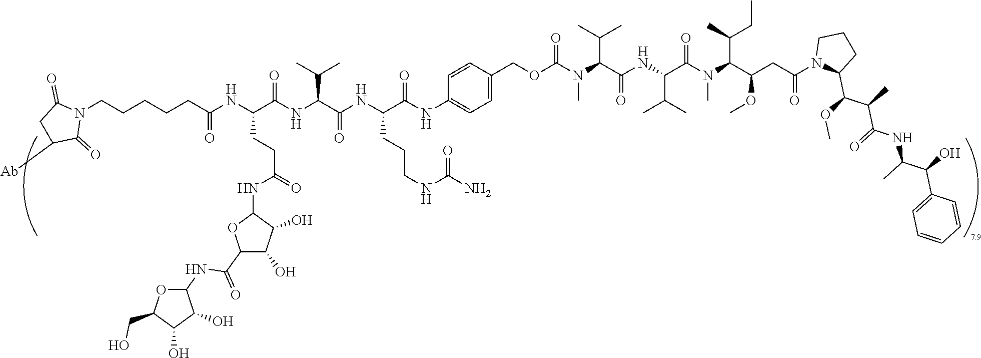 | 14.3* |
| ADC2-37 | trastu- zumab | 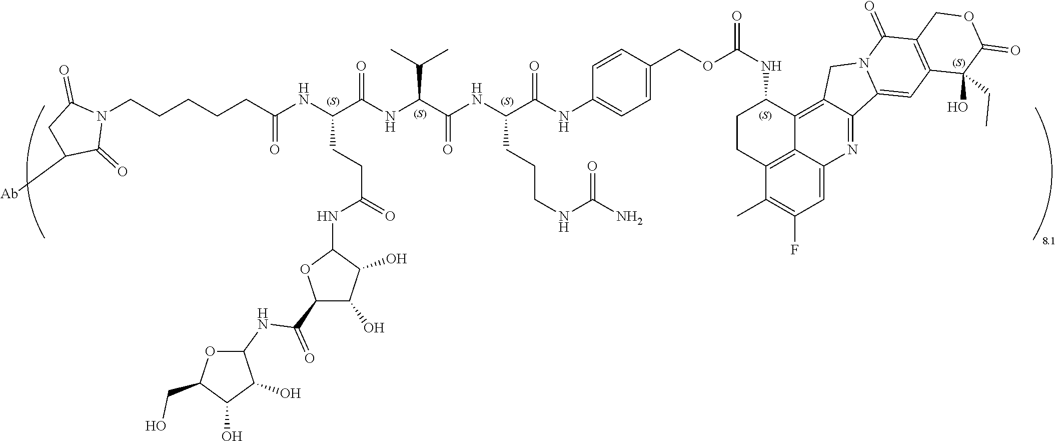 | NA |
| ADC2-38 | trastu- zumab | 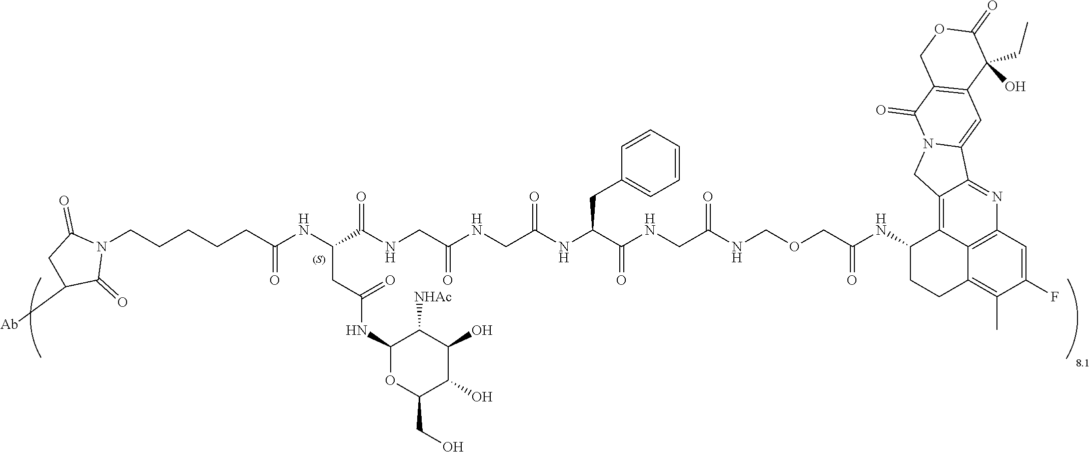 | 17.837 |
| ADC2-39 | trastu- zumab | 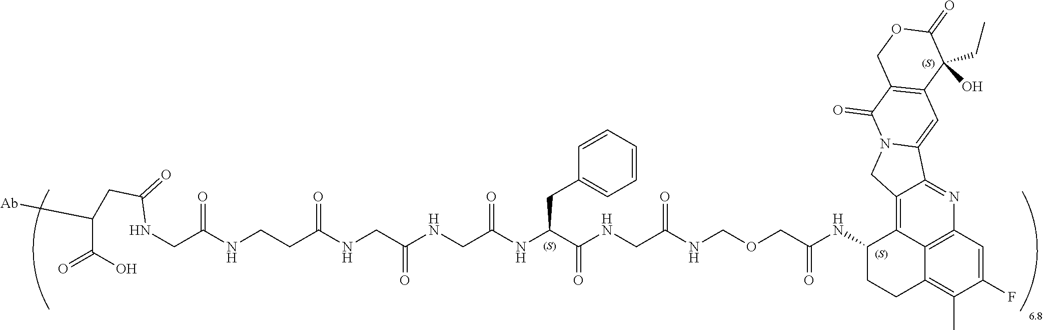 | 18.948 |
| ADC2-40 | trastu- zumab | 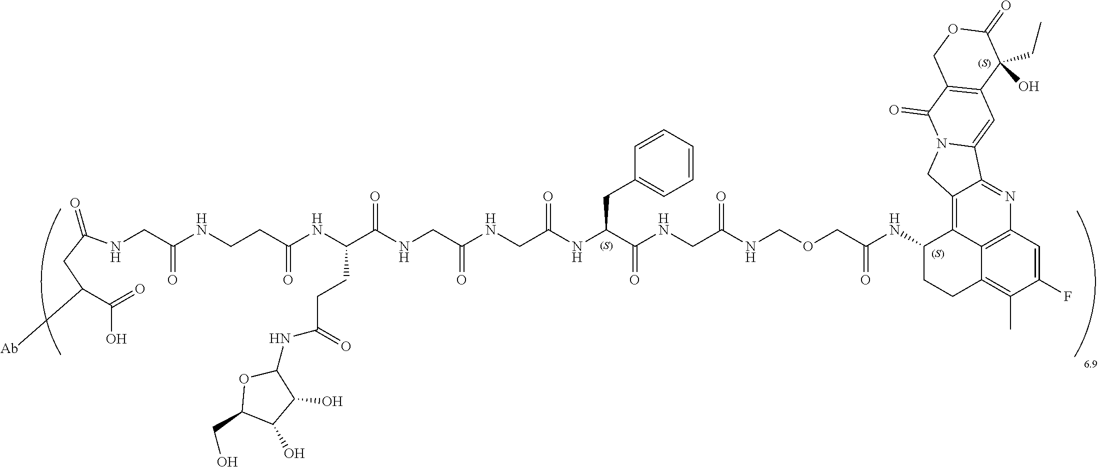 | 18.376 |
| ADC2-41 | trastu- zumab | 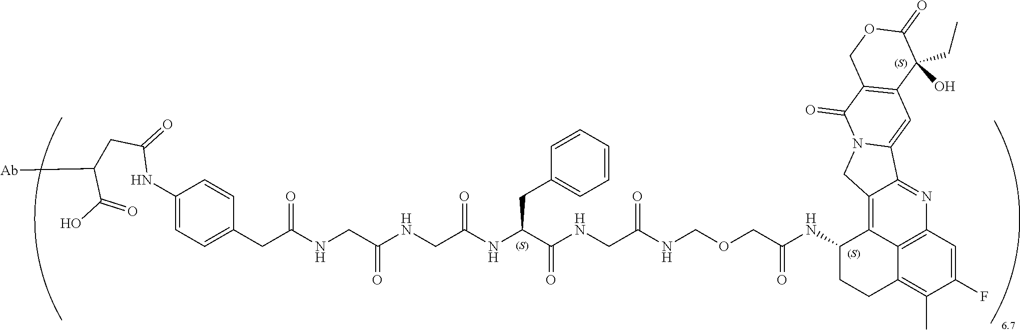 | 19.299 |
| ADC2-42 | trastu- zumab | 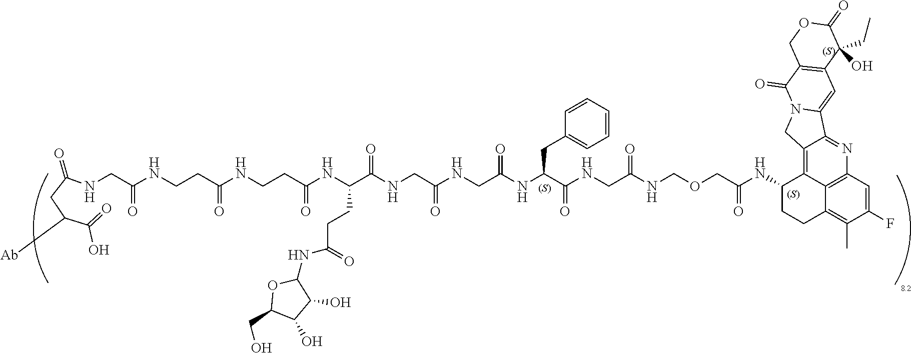 | 18.343 |
| ADC2-42- RO | trastu- zumab | 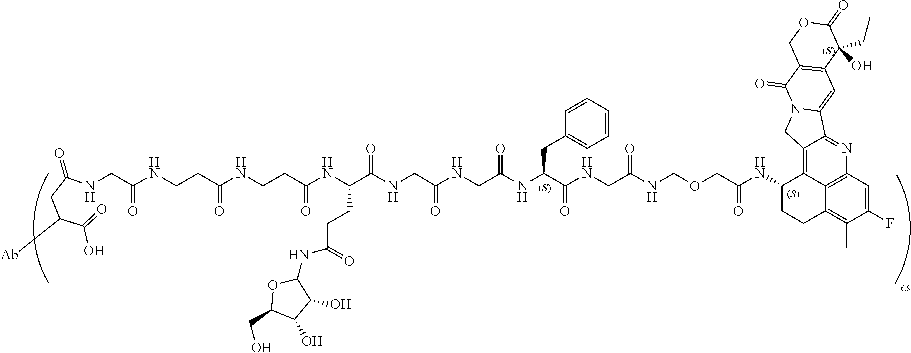 | 18.448 |
| ADC2-43 | trastu- zumab | 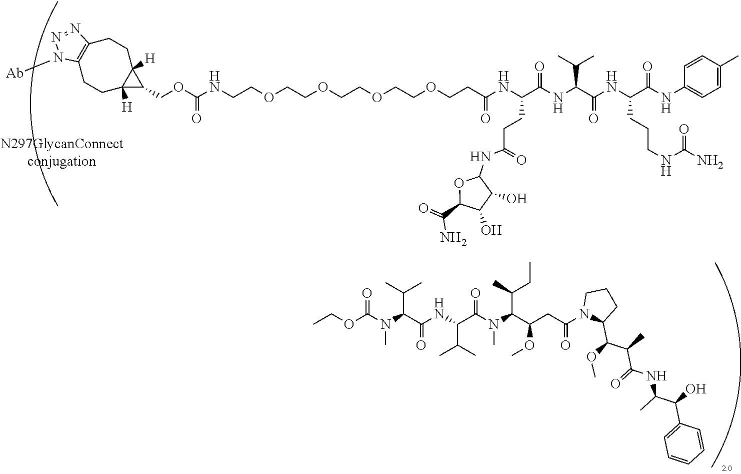 | NA |
| ADC2-44 | trastu- zumab | 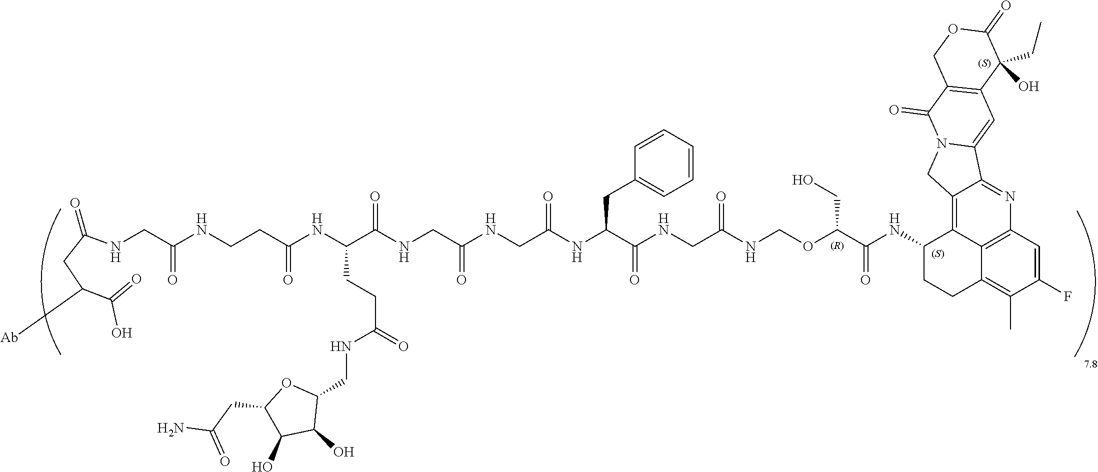 | 17.620 |
| ADC2-45 | trastu- zumab | 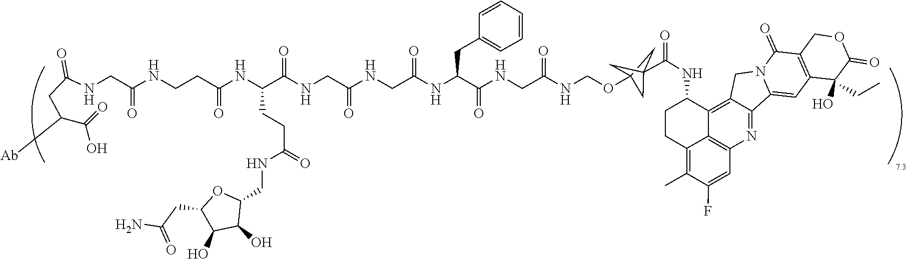 | 20.293 |
| ADC2-46 | trastu- zumab | 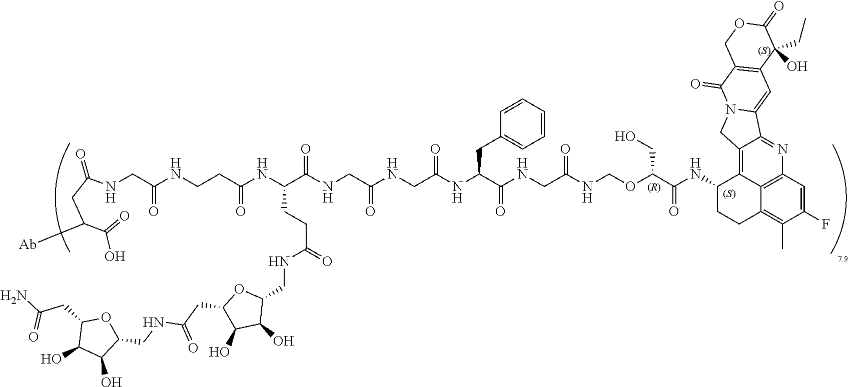 | 16.979 |
| ADC2-47 | trastu- zumab | 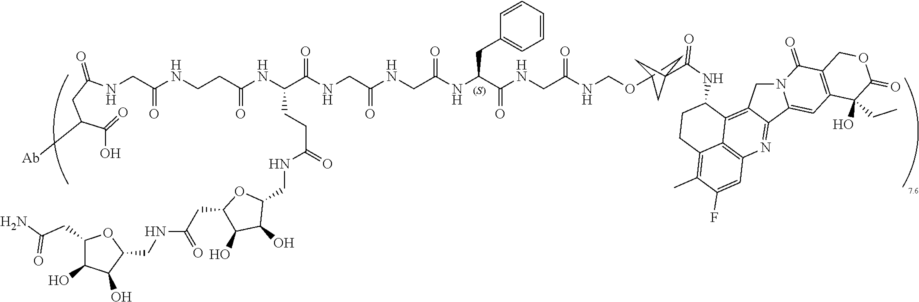 | 19.288 |
| ADC2-48 | trastu- zumab | 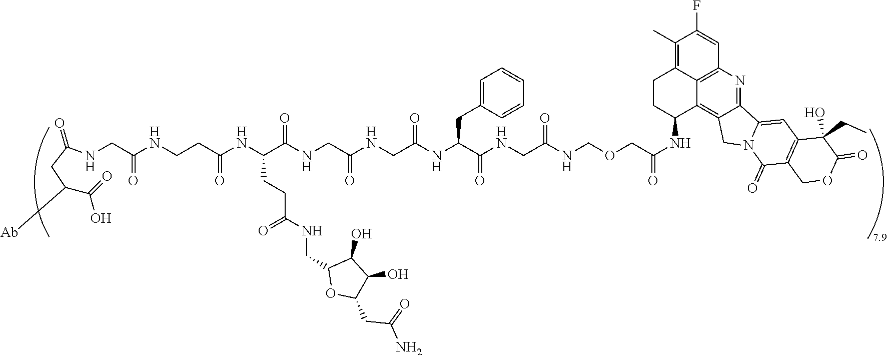 | 10.423* |
| ADC2-49 | trastu- zumab | 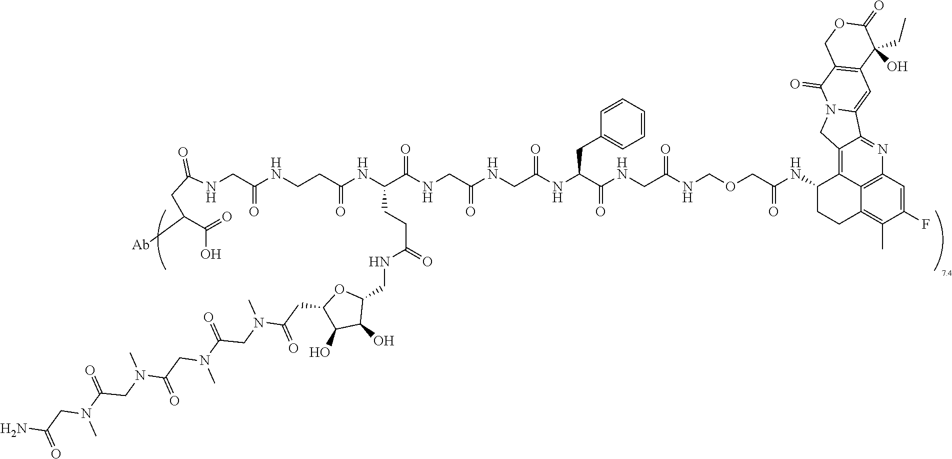 | 10.132* |
| ADC2-50 | trastu- zumab | 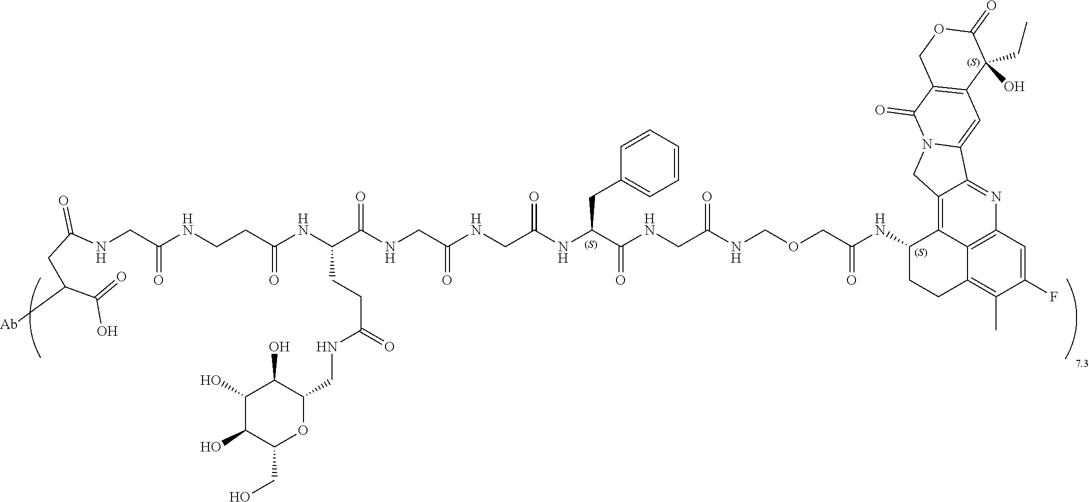 | 10.250* |
| ADC2-51 | trastu- zumab | 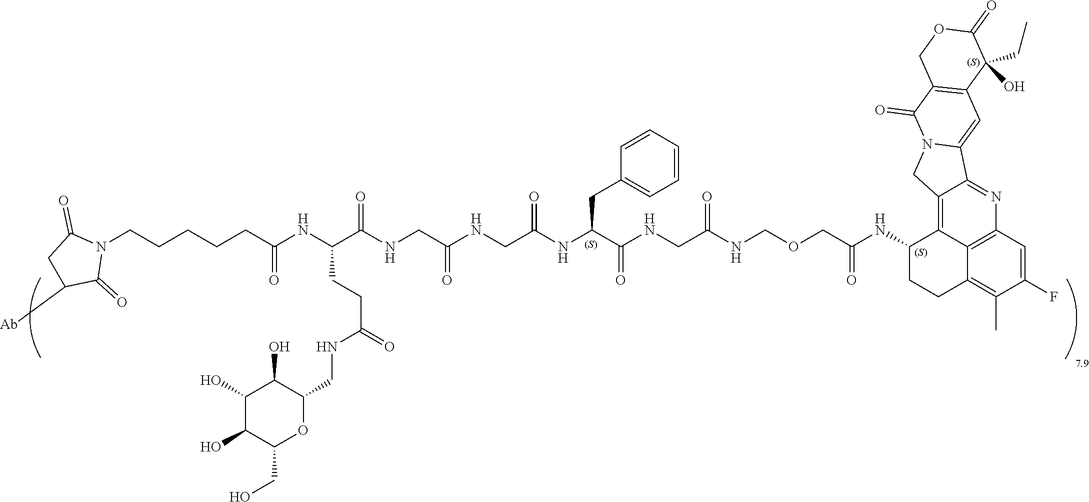 | 10.020* |
| ADC2-53 | trastu- zumab | 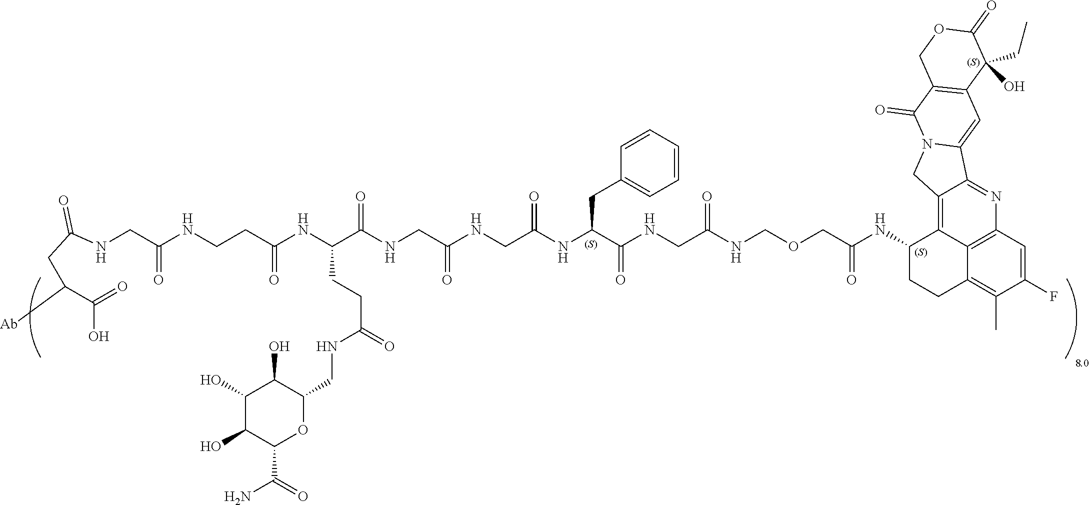 | 10.217* |
| ADC2-54 | trastu- zumab | 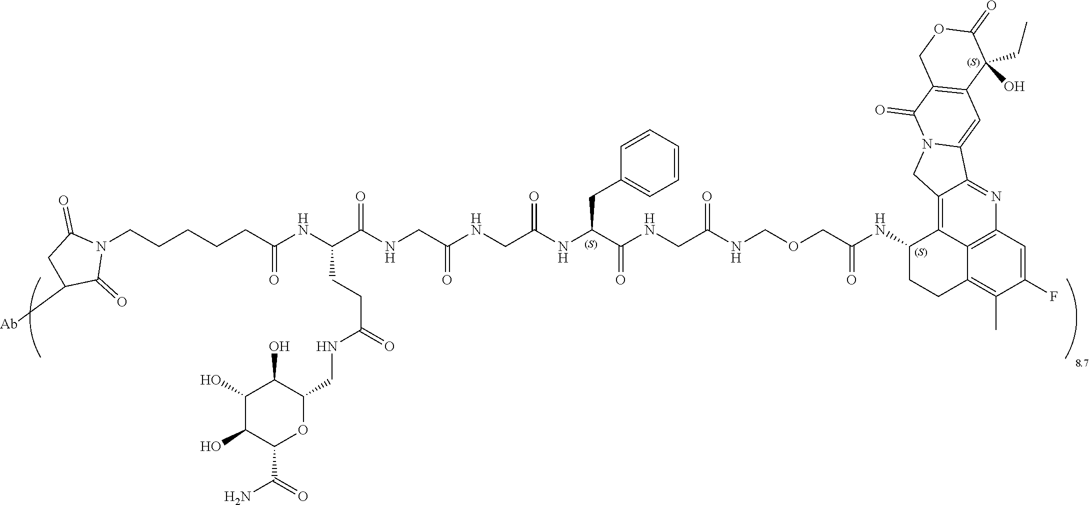 | 9.978* |
| ADC2-55 | trastu- zumab | 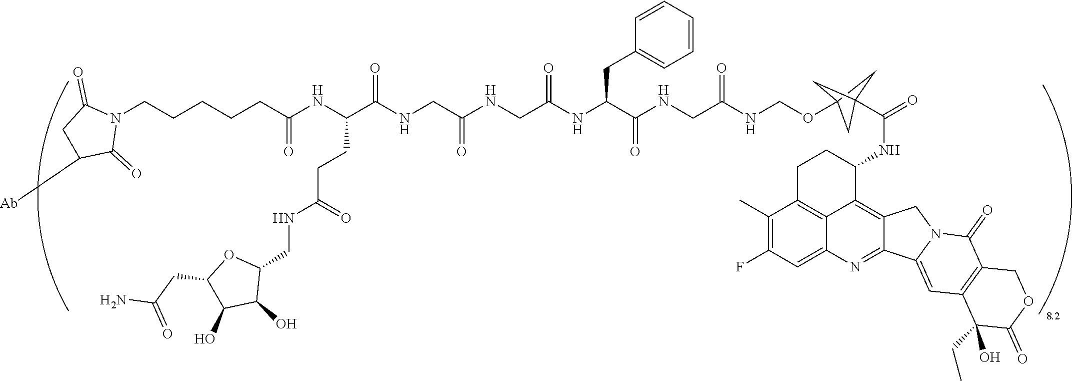 | 10.404* |
| ADC2-56 | trastu- zumab | 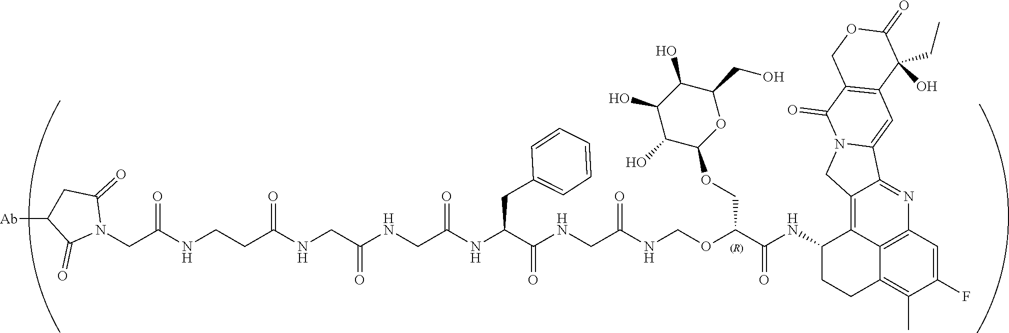 | 9.569* |
| ADC2-57 | trastu- zumab | 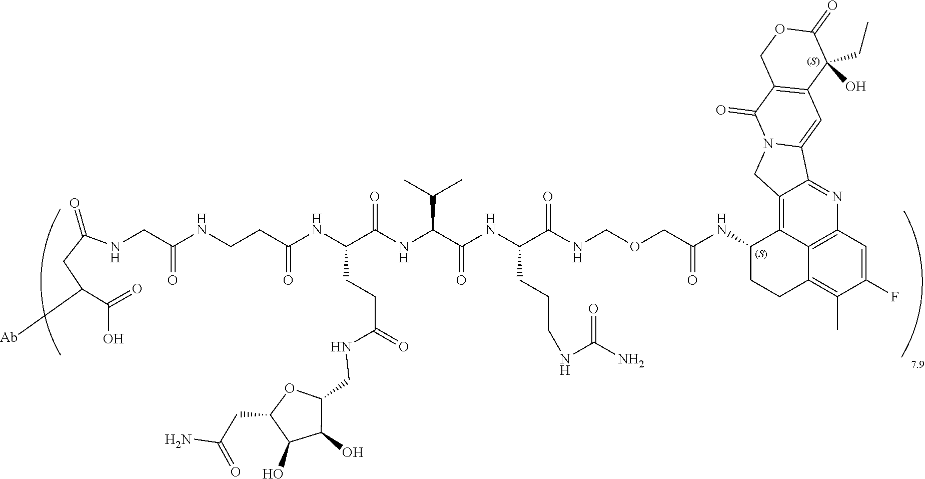 | 9.627* |
| ADC2-58 | trastu- zumab | 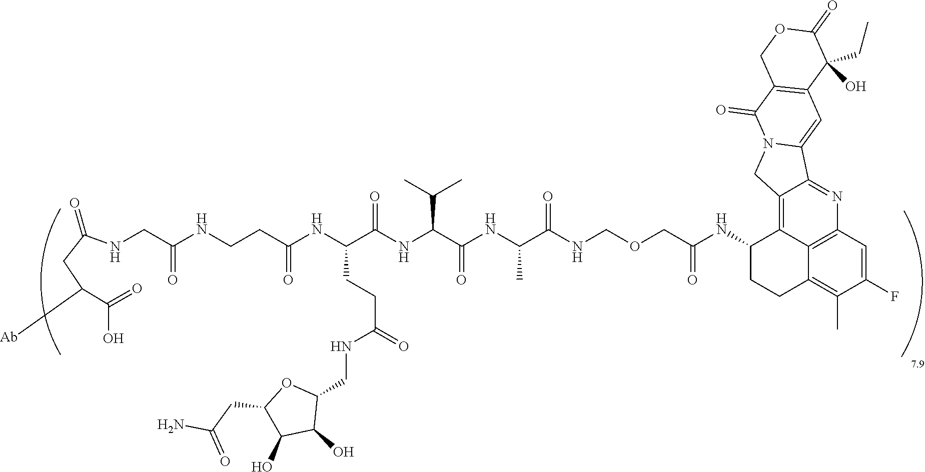 | 9.738* |
| ADC2-59 | trastu- zumab | 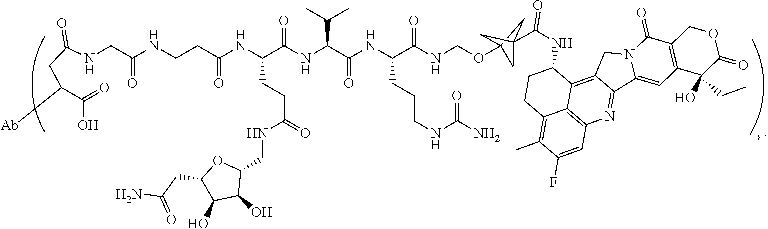 | 10.119* |
| ADC2-60 | trastu- zumab | 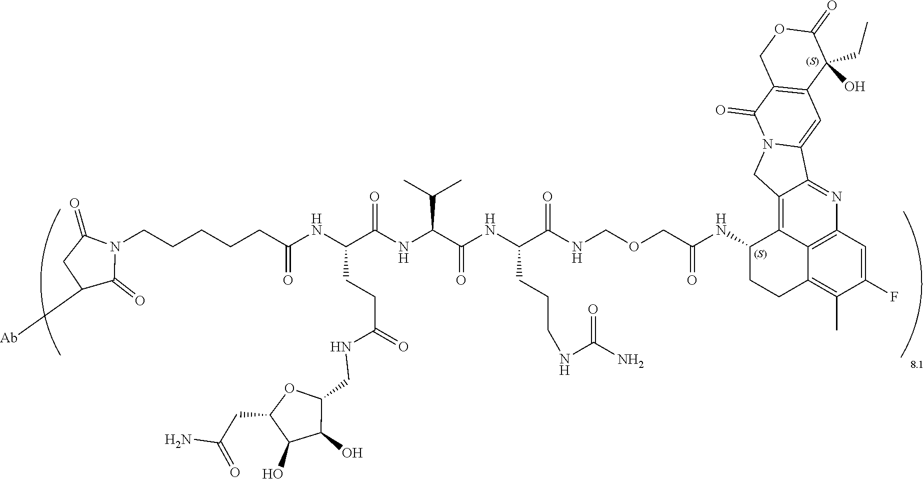 | 9.462* |
| ADC2-61 | trastu- zumab | 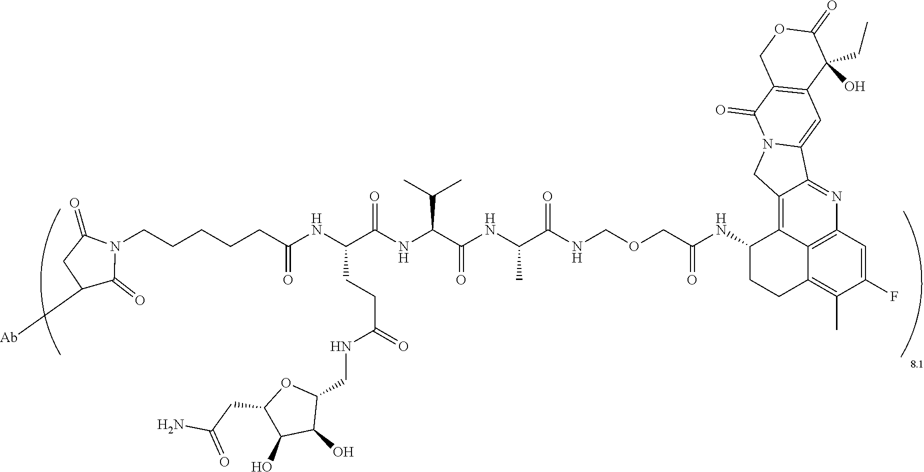 | 9.797* |
| ADC-2- 62-1 | trastu- zumab | 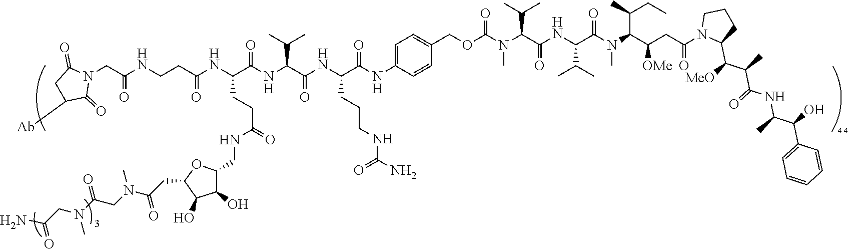 | 15.071* |
| | | 62 | |
| ADC-2- 62-2 | trastu- zumab | 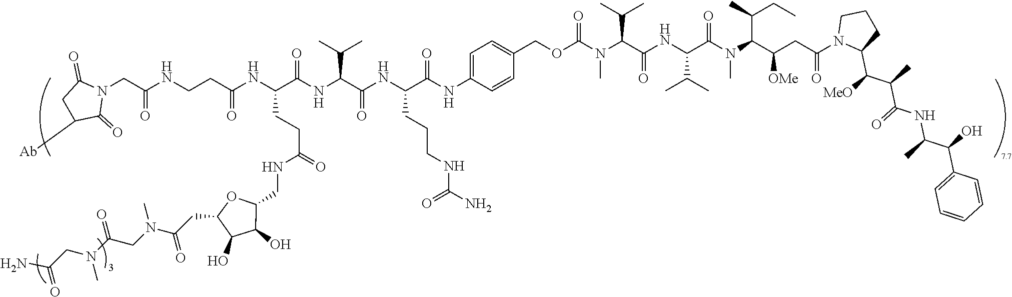 | 14.868* |
| ADC-2- 63-1 | trastu- zumab | 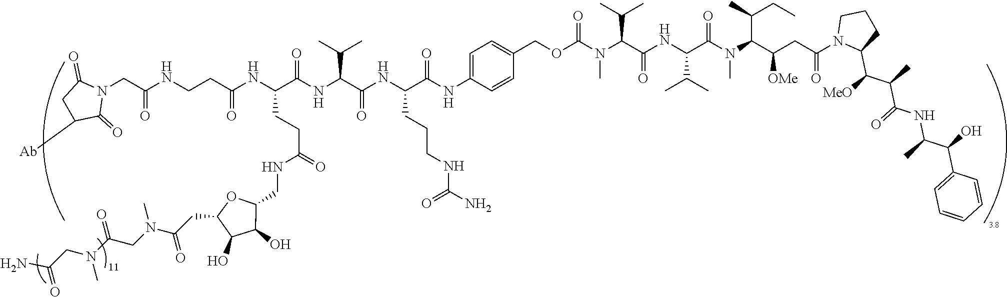 | 14.163* 4.16 |
| ADC-2- 63-2 | trastu- zumab | 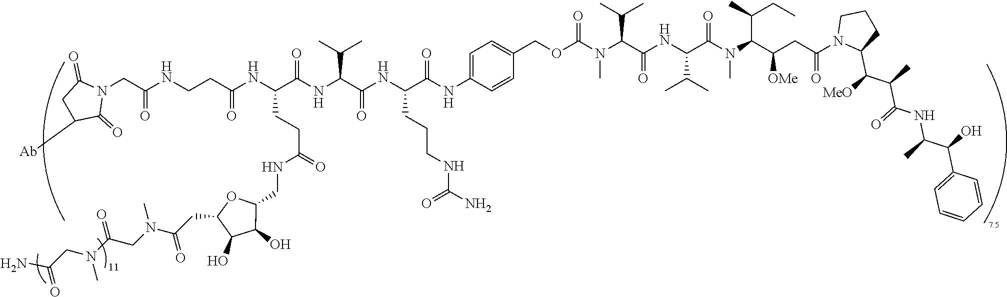 | 13.955* |
| ADC-2- 64-1 | trastu- zumab | 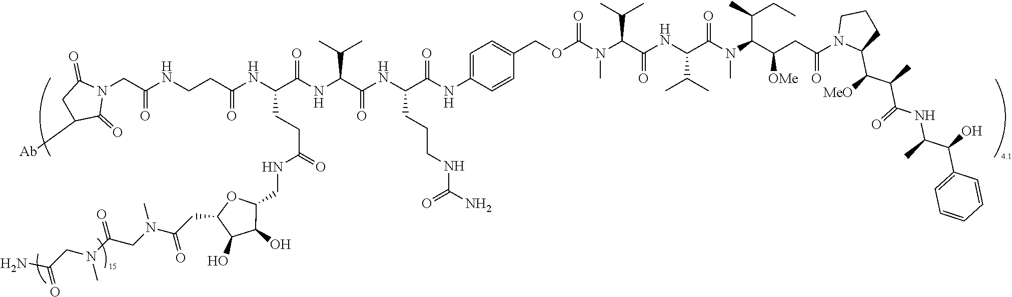 | 13.728* |
| ADC-2- 64-2 | trastu- zumab | 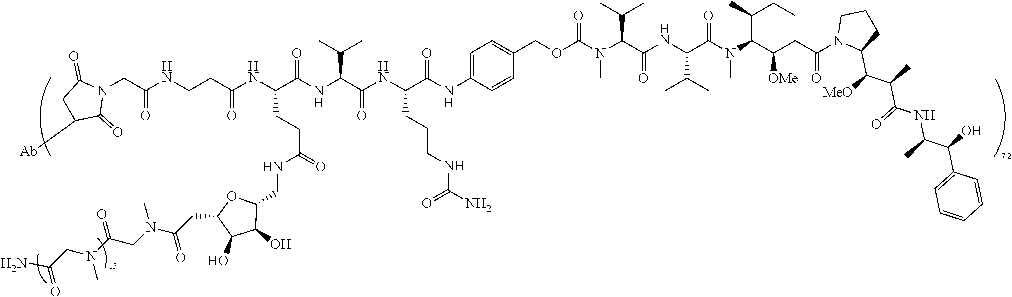 | 13.678* |
| ADC2-65-1 | trastu- zumab | 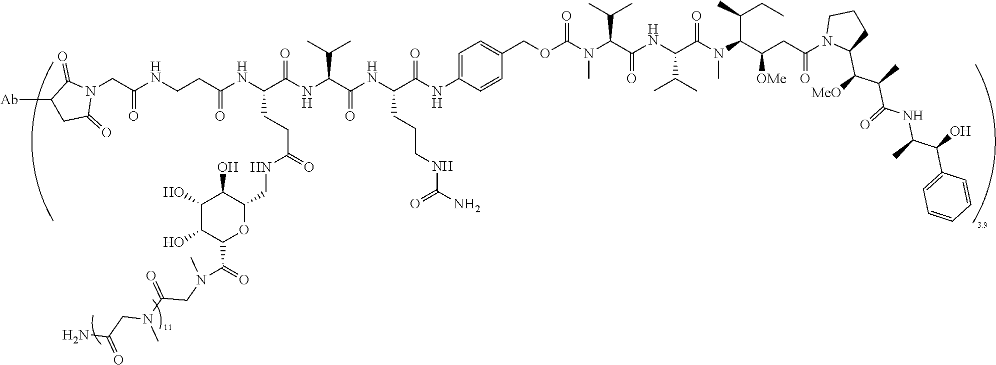 | 14.037* |
| ADC2-65-2 | trastu- zumab | 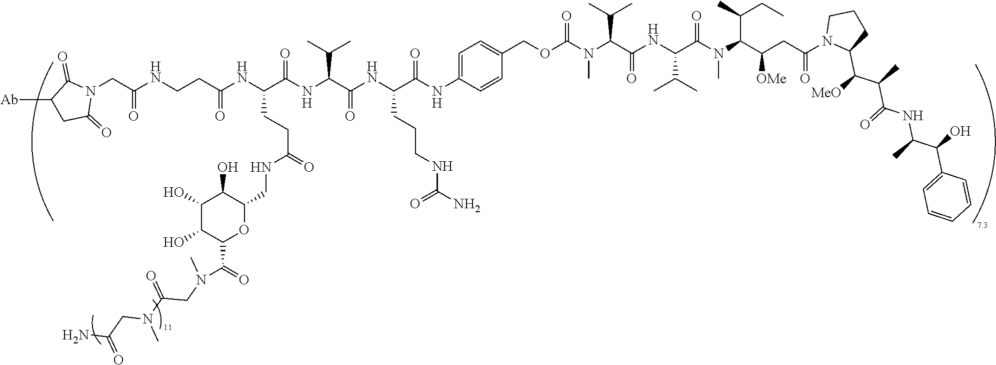 | 13.913* |
| ADC3-1 | patritu- mab | 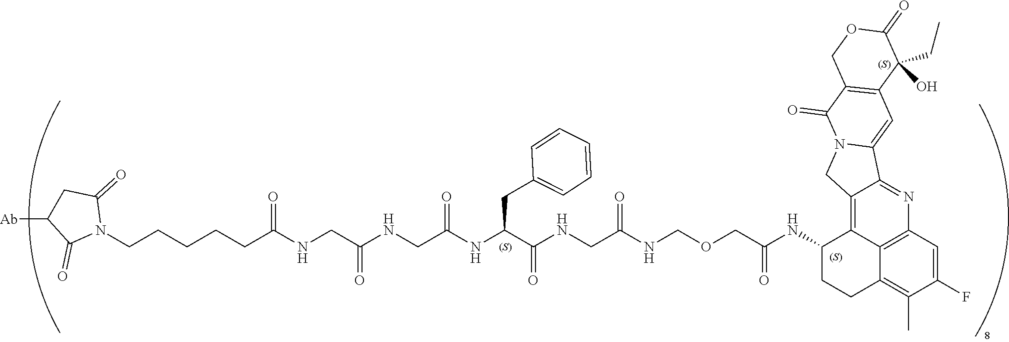 | 10.714* 21.074 |
| ADC3-2 | patritu- mab | 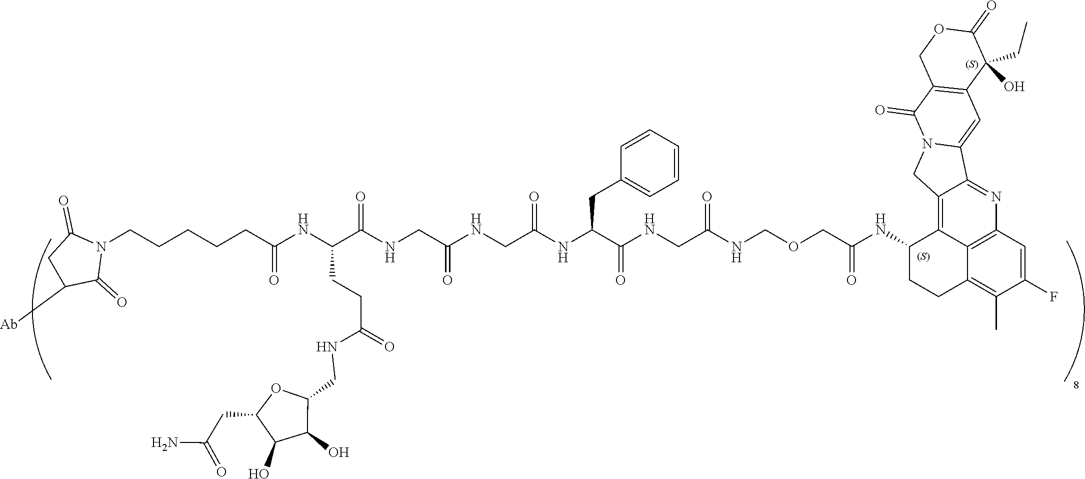 | 19.628 |
| ADC3-3 | patritu- mab | 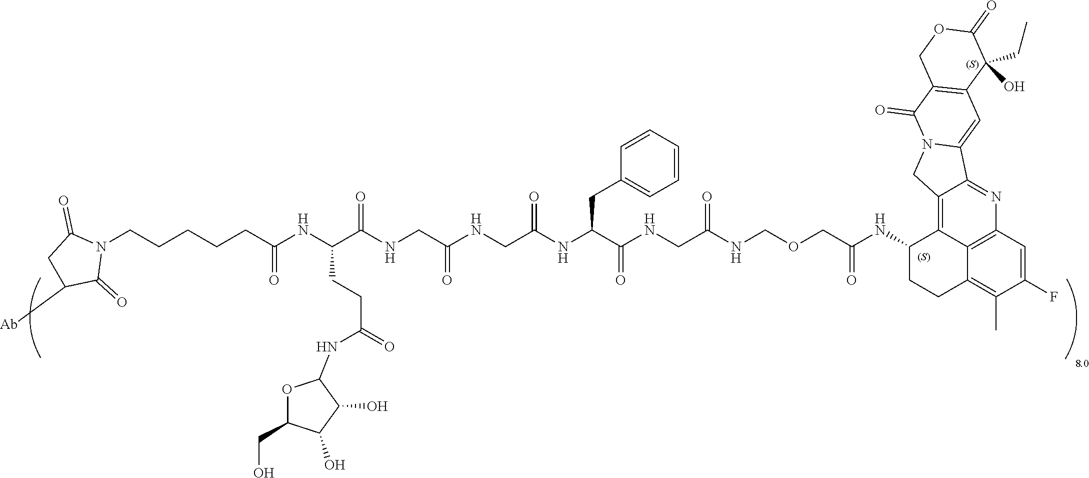 | 19.642 |
| ADC3-4 | patritu- mab | 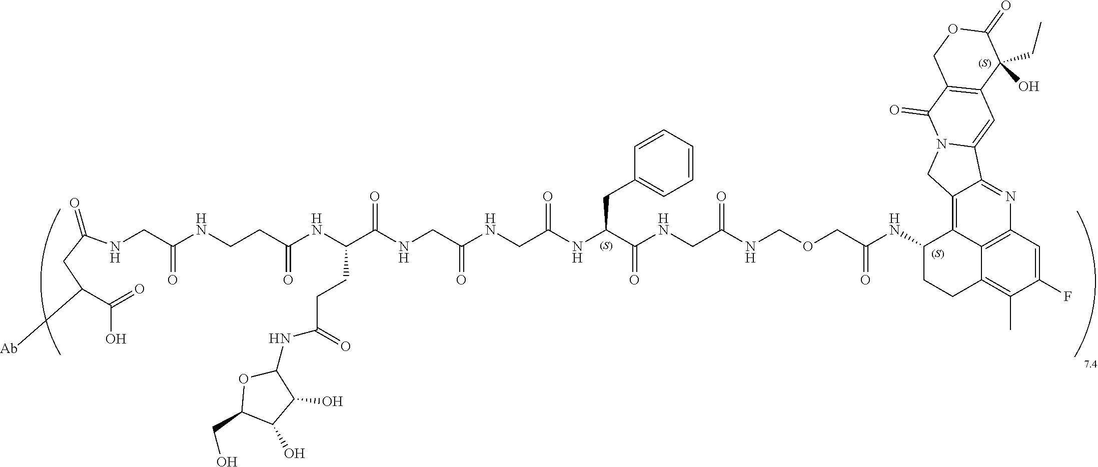 | 18.966 |
| ADC3-5 | patritu- mab | 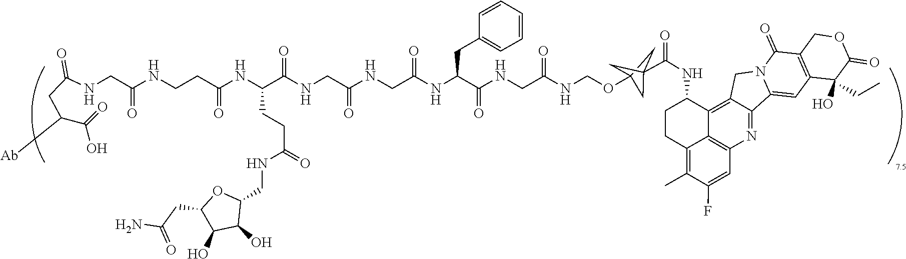 | 20.909 |
| ADC3-6 | patritu- mab | 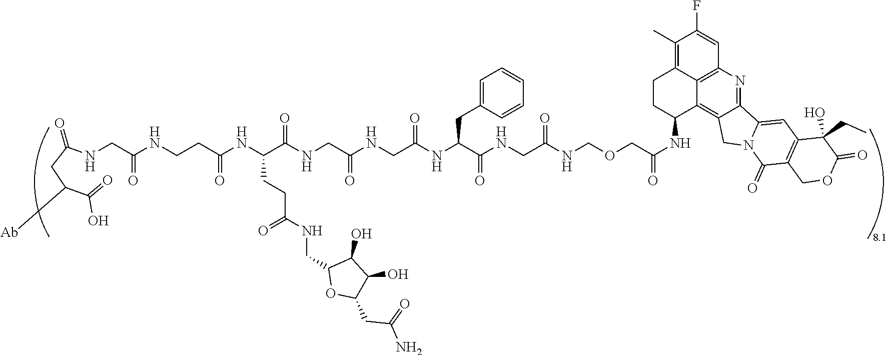 | 10.602* |
| ADC3-7 | patritu- mab | 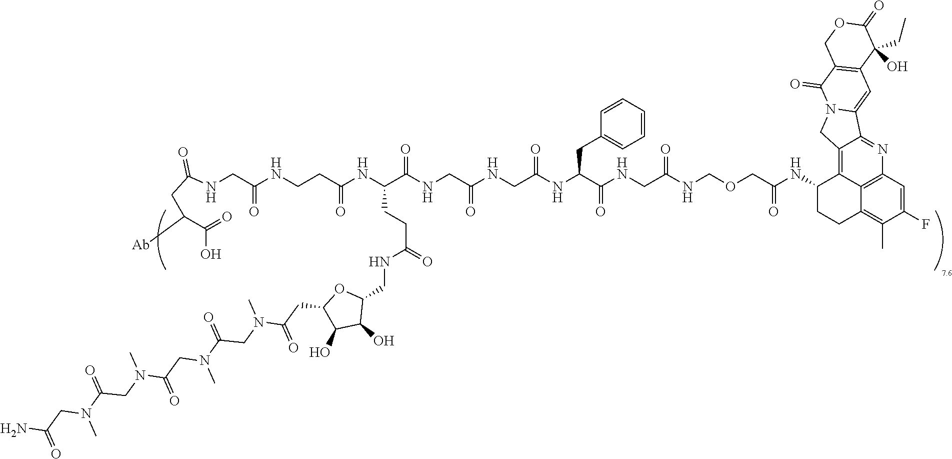 | 18.946 |
| ADC3-8 | patritu- mab | 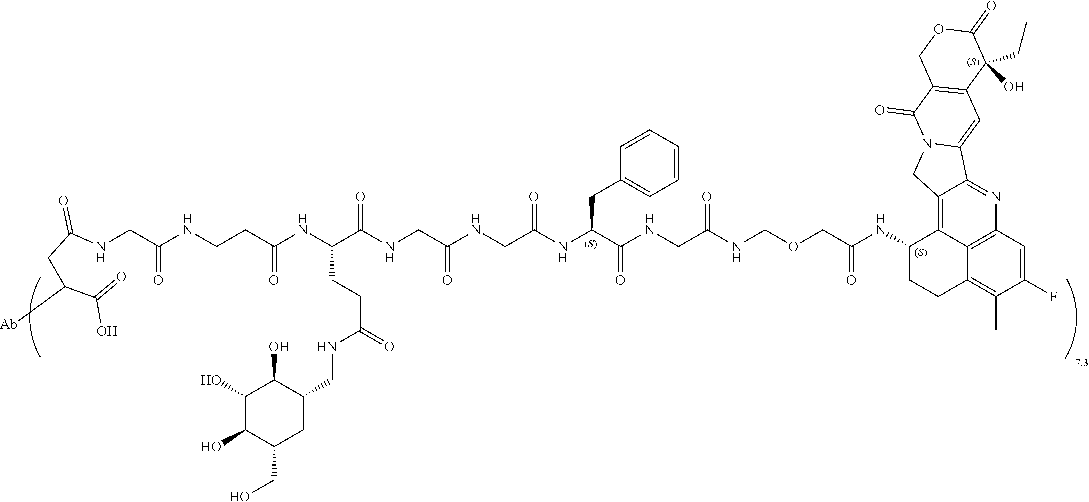 | 19.135 |
| ADC3-9 | patritu- mab | 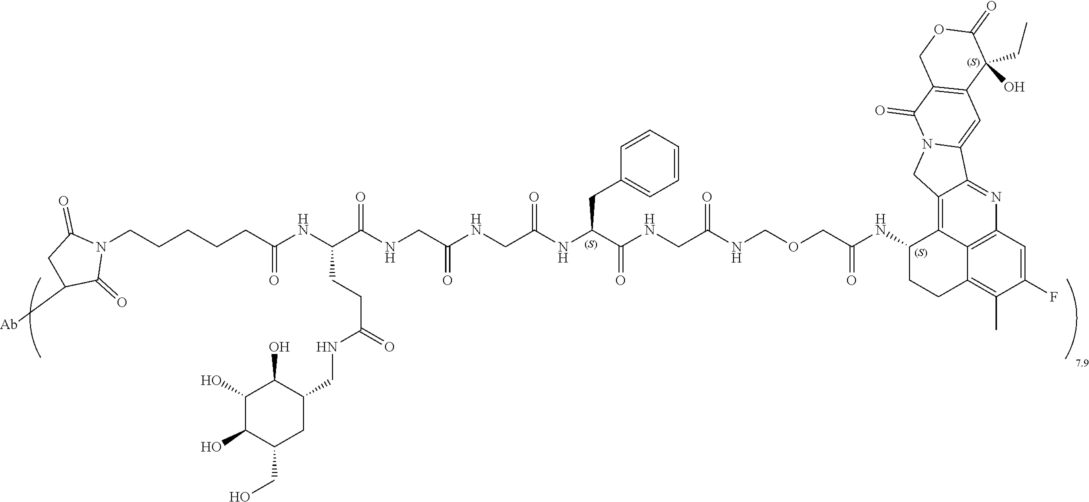 | 19.312 |
| ADC3-10 | patritu- mab | 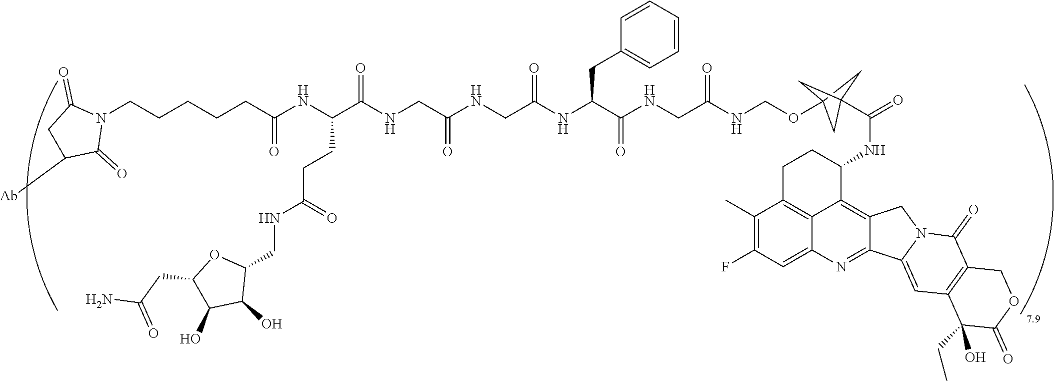 | 20.783 |
| ADC3-11 | patritu- mab | 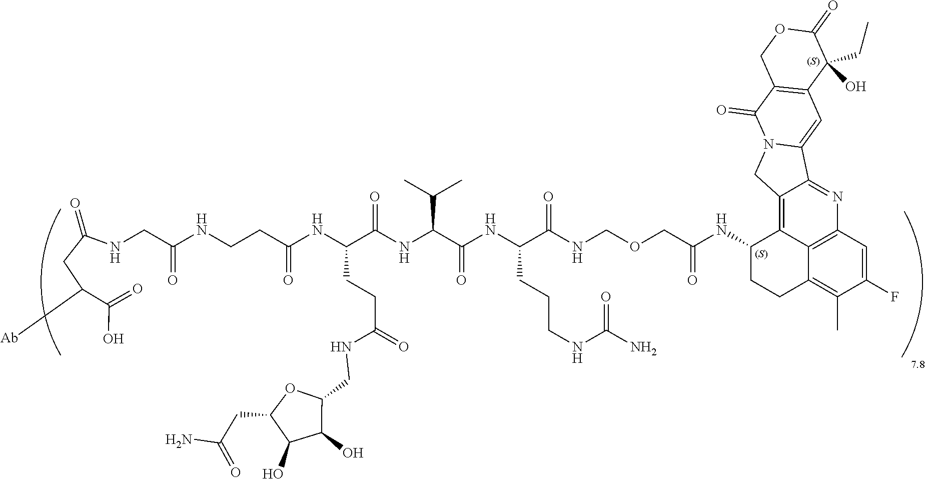 | 18.524 |
| ADC3-12 | patritu- mab | 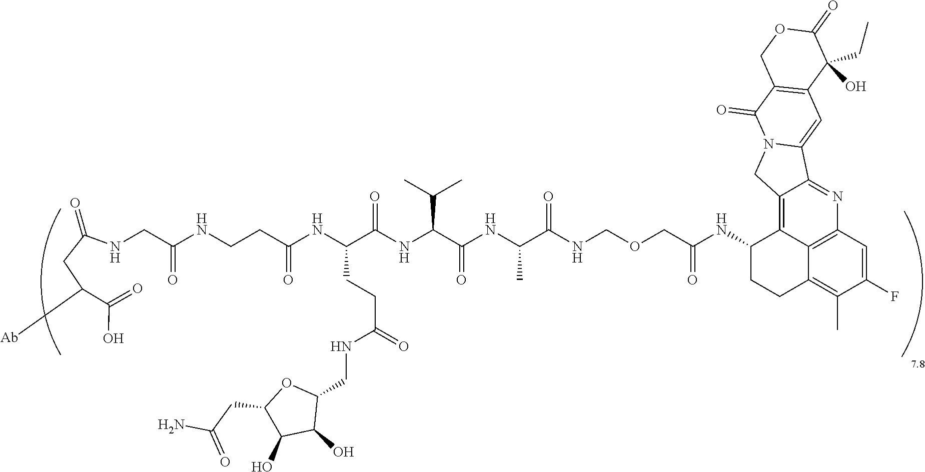 | 18.576 |
| ADC3-13 | patritu- mab | 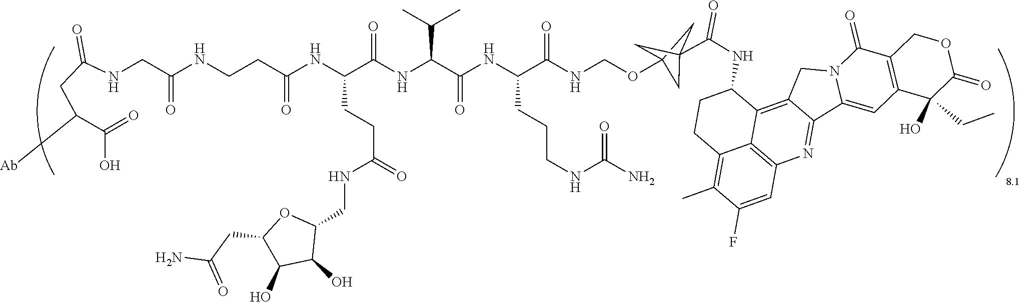 | 10.318* |
| ADC3-14 | patritu- mab | 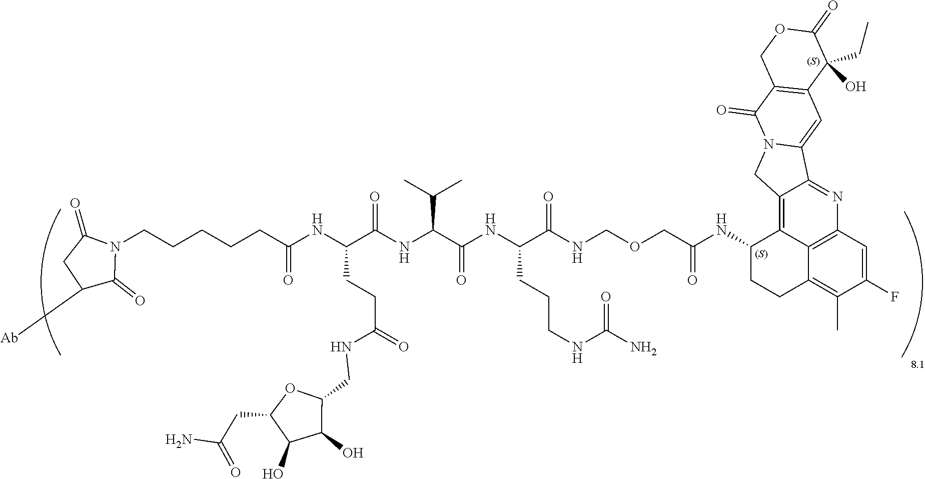 | 18.540 |
| ADC3-15 | patritu- mab | 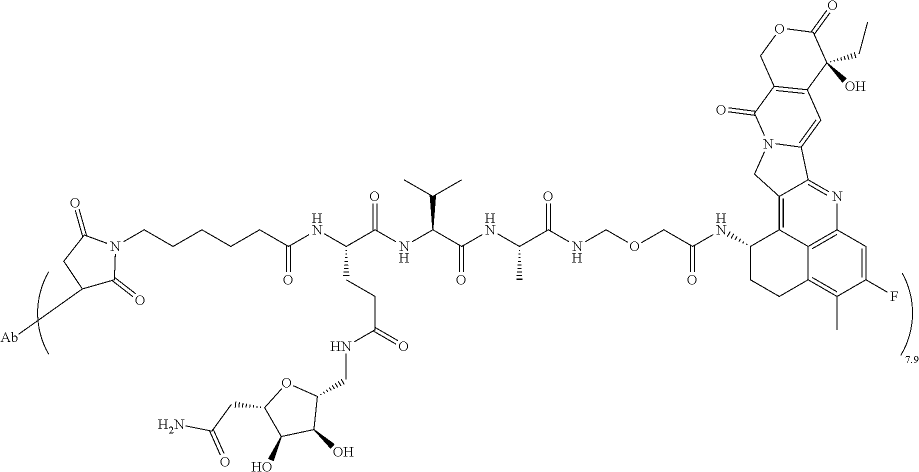 | 9.577* |
| ADC3-16 | patritu- mab | 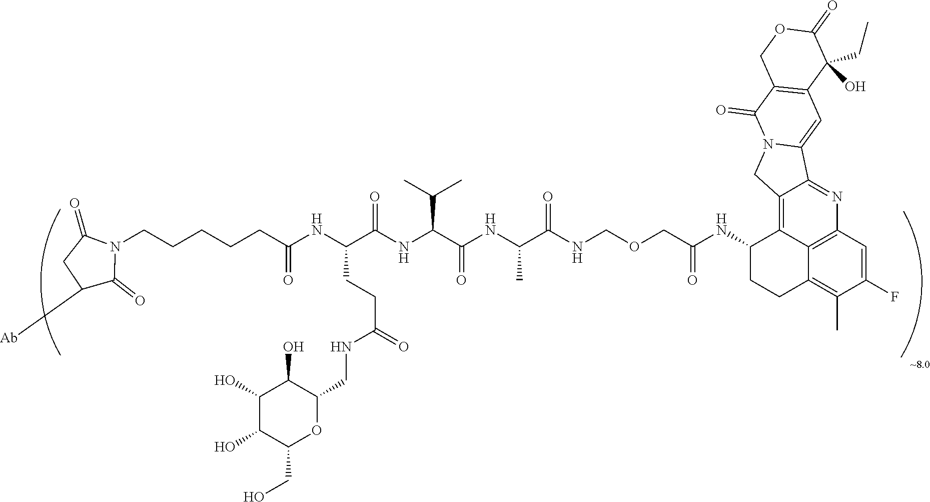 | 9.607* |
| ADC3-17 | patritu- mab | 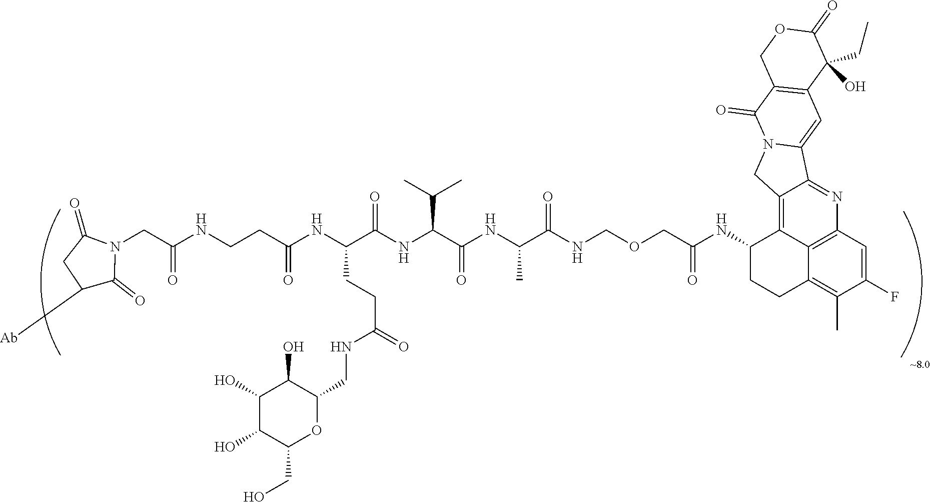 | 9.816* |
| ADC3-18 | patritu- mab | 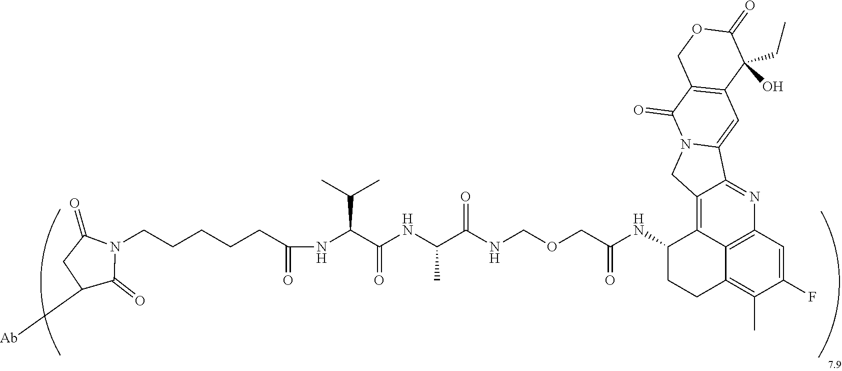 | 9.967* |
| ADC3-19 | patritu- mab | 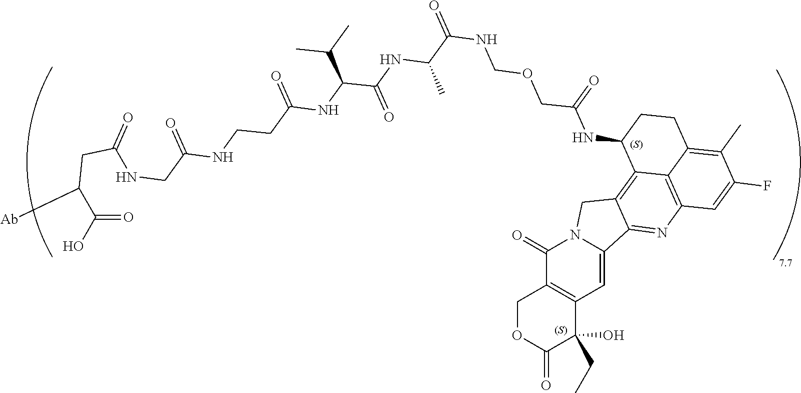 | 9.809* |
| ADC3-20 | patritu- mab | 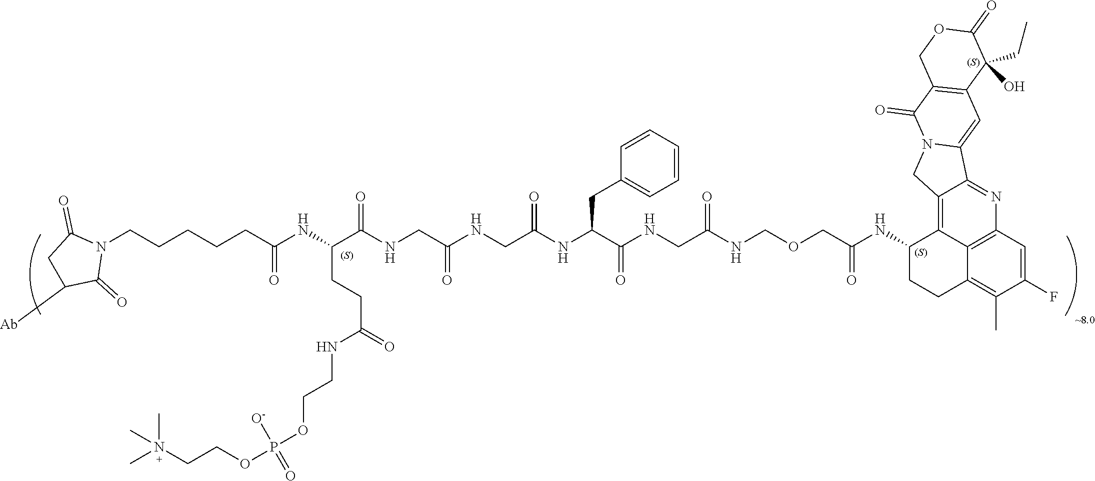 | 9.517* |
| ADC-C2 | Patritu- mab | 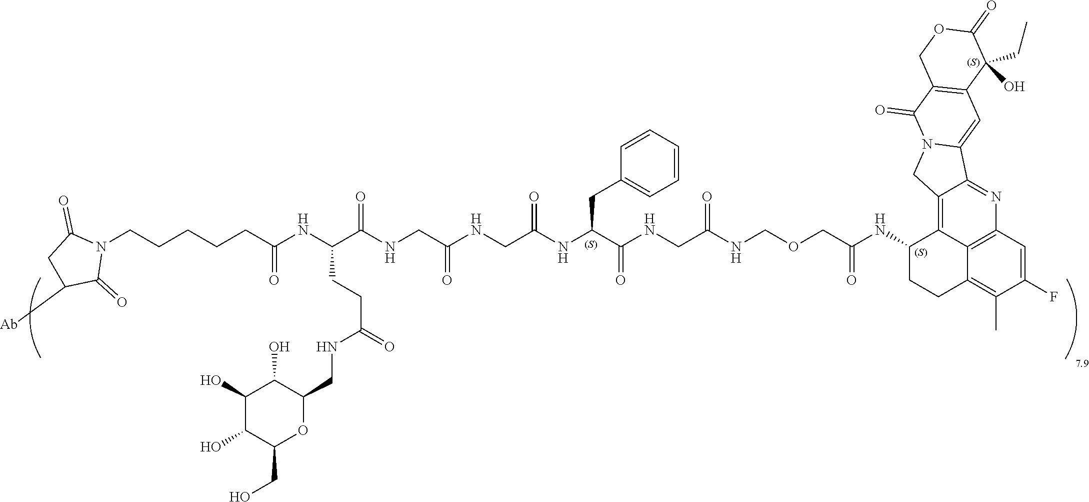 | Method 1: 10.233 Method 2: 19.312 |
| ADC-C3 | Patritu- mab | 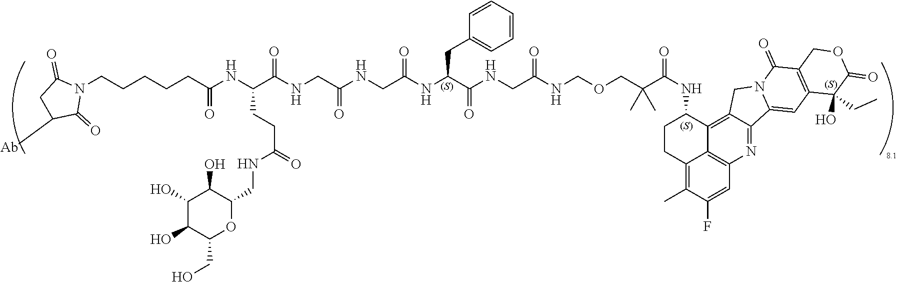 | Method 1: 10.319 |
| ADC-C4 | Patritu- mab | 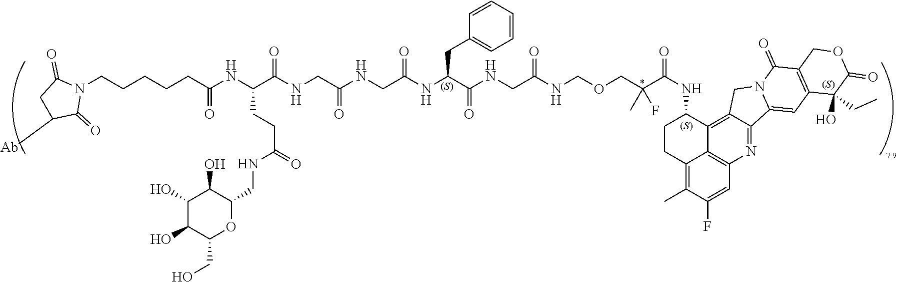 | Method 1: 9.627 |
| | | payload is isomer 1 | |
| ADC-C5 | Patritu- mab | 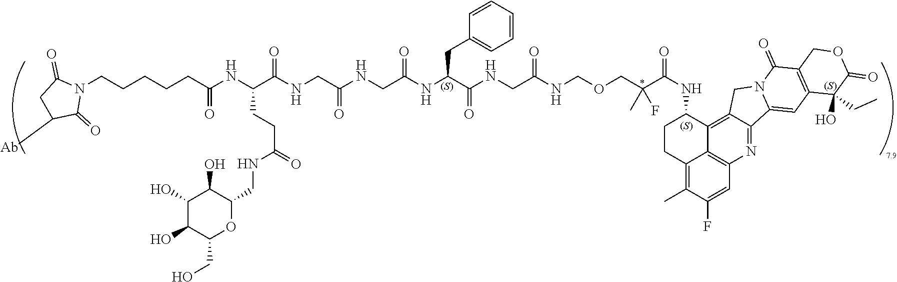 | Method 1: 10.029 |
| | | payload is isomer 2 | |

*DAR8 species (min) in above table was determined by HIC method with isopropanol in the mobile phase.

TABLE 4
ADC Compounds used references

| | | | HIC |
|---|---|---|---|
| | | | DAR8 |
| | | | RT |
| Examples | Antibody | Antibody drug conjugate | (min) |
| ADC1-1 | Cofetuzumab | 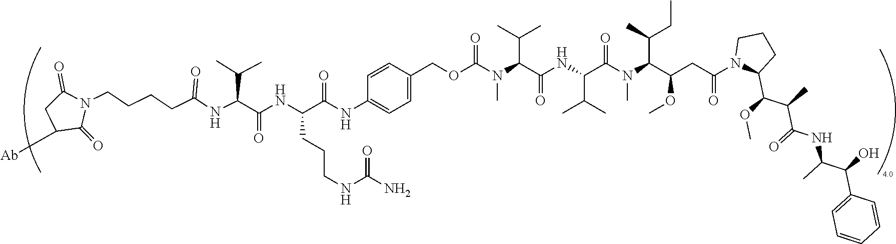 | 14.56* |
| ADC1-12 | Cofetuzumab | 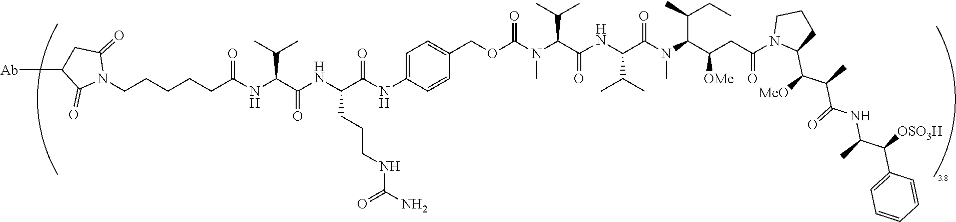 | 13.98* |
| ADC2-A | trastuzumab | 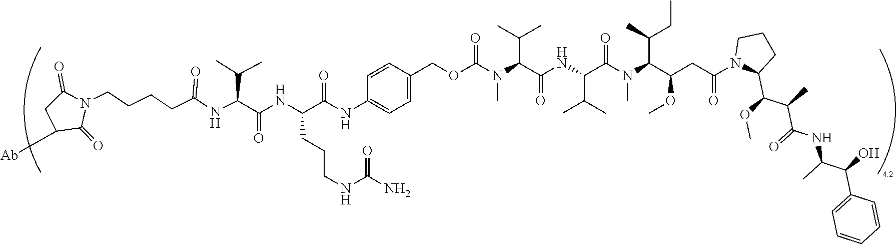 | 14.6* |
| ADC2-B | trastuzumab | 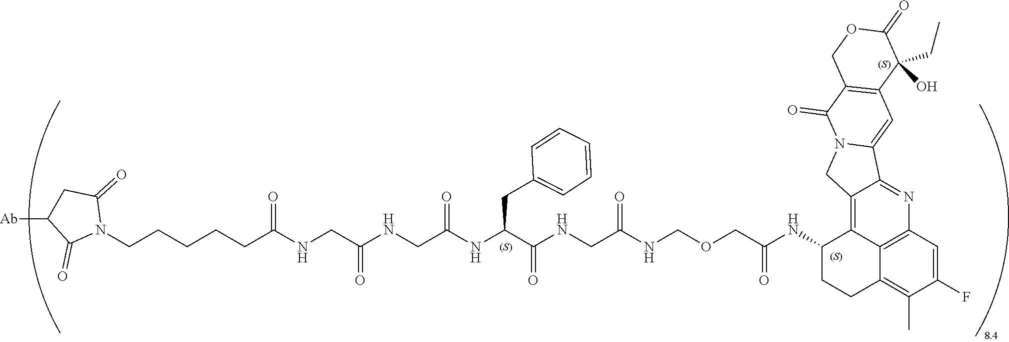 | 10.186*/ 20.197 |
| ADC2-12 | trastuzumab | 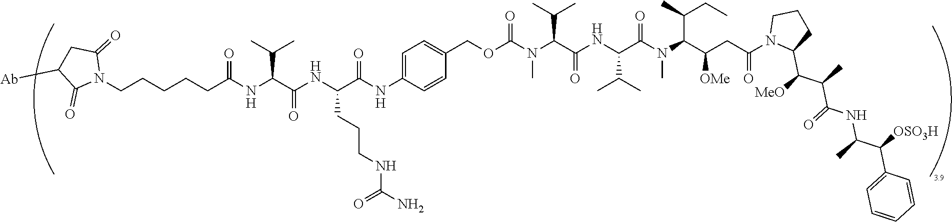 | 13.7* |
| ADC-C1 | Patritumab | 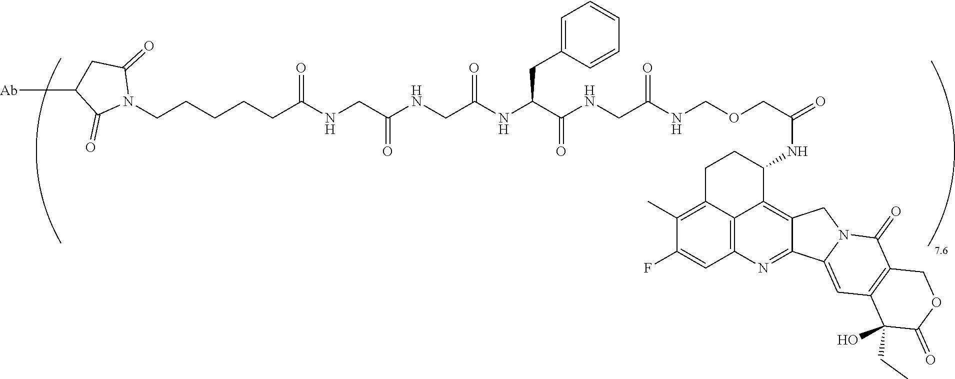 | Method 1: 10.714 Method 2: 21.074 Note: used as a reference |

The HIC retention time (HIC RT, here only HIC RT of DAR8 species is listed in the table) can be seen as a measure to evaluate ADC's hydrophobicity. Shorter HIC RT indicates that ADC is more hydrophilic. As provided herein, the P5-modification GGFG-aminal-Dxd base ADCs have shorter HIC RT compared with general MC-GGFG-aminal-Dxd ADC. Moreover, the P3-modification-VA/VC/VAG-aminal-Dxd ADCs appeared to have much shorter HIC RT than general MC-GGFG-aminal-Dxd ADCs. For highly hydrophobic MMAE base ADCs, the P3-modification-VC-PAB-MMAE ADCs appeared to have shorter DAR8 HIC RT than general MC-VC-PAB-MMAE ADCs. It is widely reported that hydrophobic linker-payload conjugated on antibodies with higher DAR would sometimes lead to ADC aggregation (low SEC purity). This property would further affect antibody binding with its target and ADC aggregation tendency would also result in its fast clearance in aminal models ((1) Clin Cancer Res 2004, 10, 7063-7070. (2) Mol. Pharmaceutics 2021, 18, 3, 889-897). Thus, it is surprising and unexpected to discover that the P3 and P5 modification improves the hydrophilic properties of ADCs, which prevents ADC aggregation (especially MMAE DAR8 ADCs), and fast clearance.

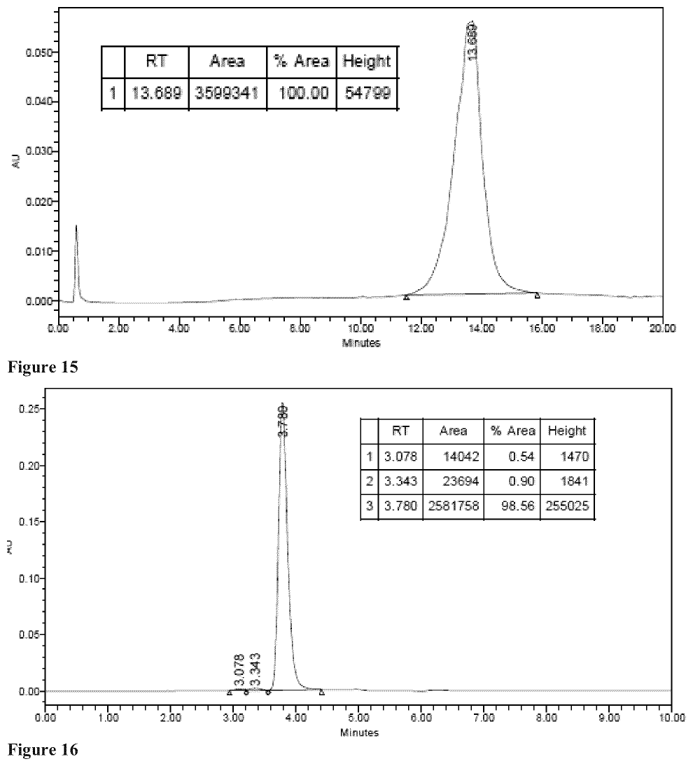

ADCs with invention modification on P3 and P5 have shown earlier retention time of DAR8 species, which are indicating better hydrophilicity properties. MMAE base ADCs (ADC2-6-2, ADC2-9-2, ADC2-36-2, ADC2-62-2, ADC2-63-2, ADC2-64-2, and ADC2-65-2) can achieve 8 drug-loaded profiles without inducing significant aggregate formation after conjugations (FIG. 15 to FIG. 20 and FIG. 62 to FIG. 69).

ADC 2-6-2 HIC profile (HIC DAR 8.0) is illustrated in FIG. 15.

ADC 2-6-2 SEC profile with >98% purity is illustrated in FIG. 16.

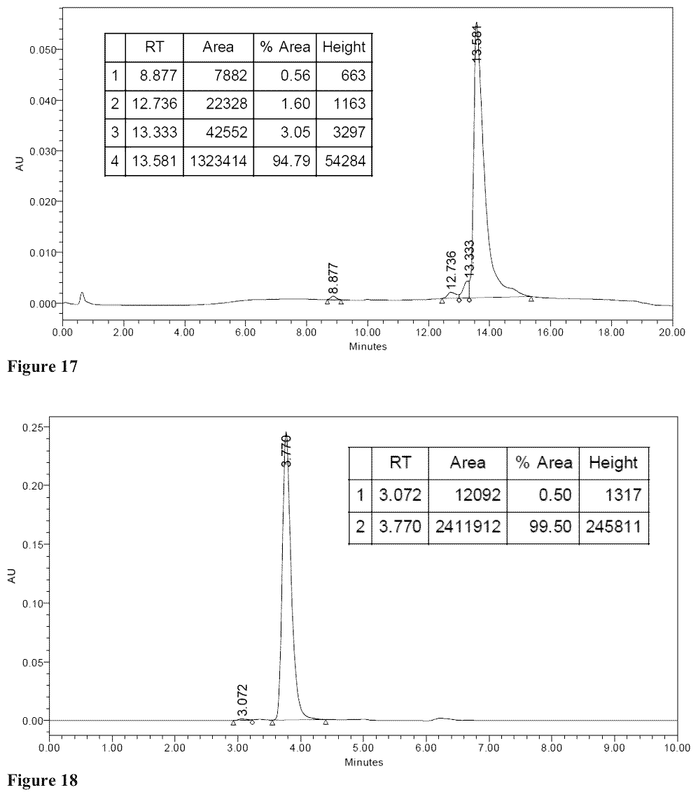

ADC 2-9-2 HIC profile (HIC DAR 8.0) is illustrated in FIG. 17.

ADC 2-9-2 SEC profile (>99% purity) is illustrated in FIG. 18.

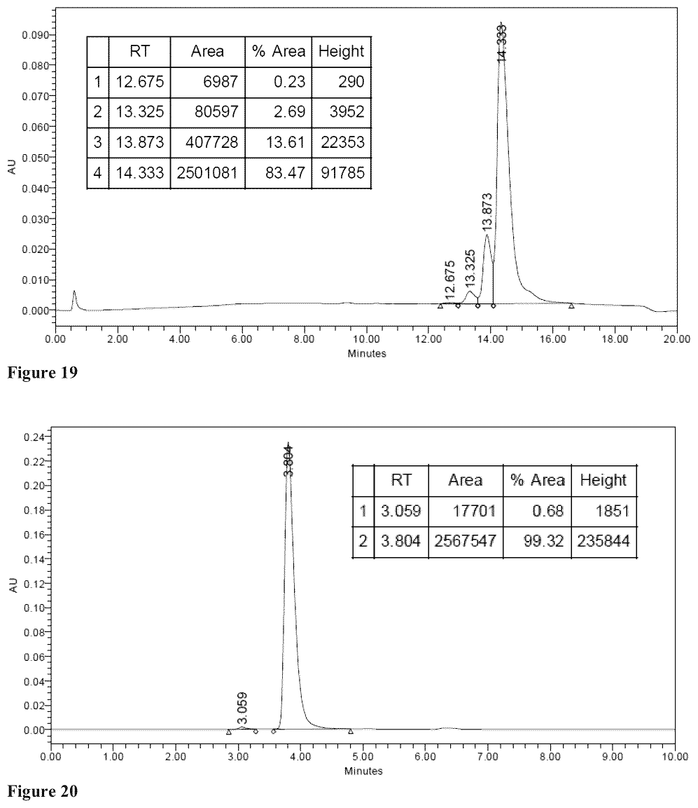

ADC2-36-2 HIC profile (HIC DAR7.8) is illustrated in FIG. 19.

ADC2-36-2 SEC profile with >99% purity is illustrated in FIG. 20.

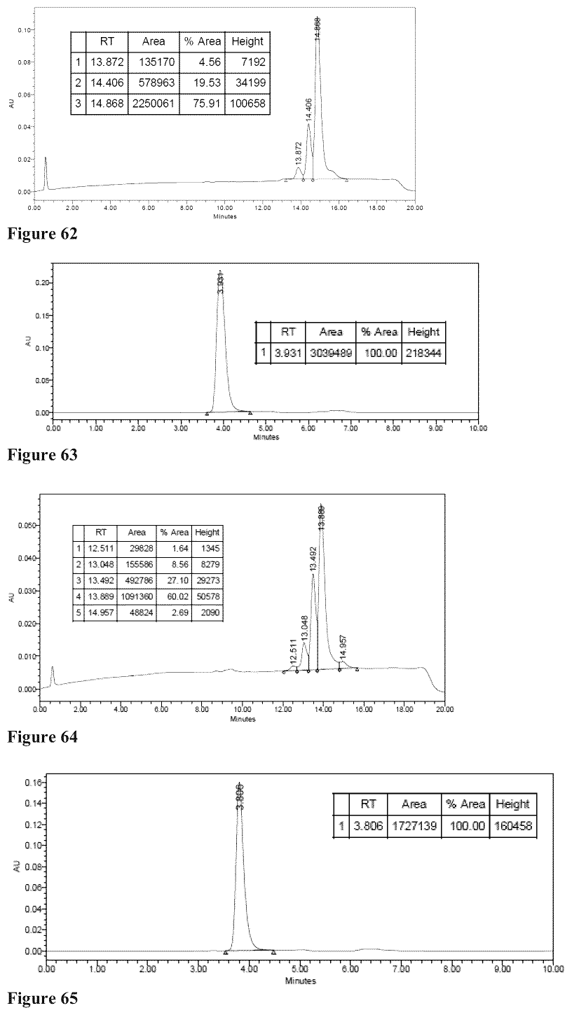

ADC2-62-2 HIC profile (HIC DAR 7.7) is illustrated in FIG. 62.

ADC2-62-2 SEC profile with >99% purity is illustrated in FIG. 63.

ADC2-63-2 HIC profile (HIC DAR 8.0) is illustrated in FIG. 64.

ADC2-63-2 SEC profile with >99% purity is illustrated in FIG. 65.

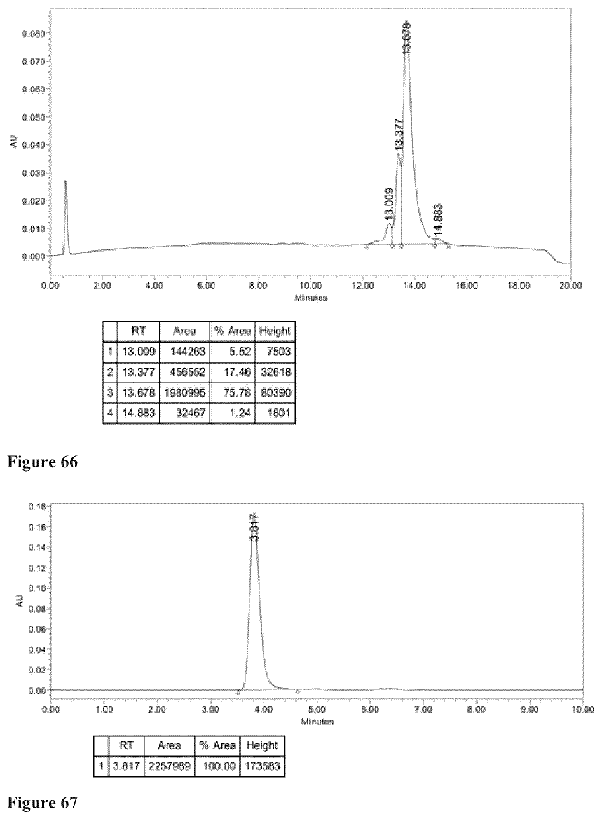

ADC2-64-2 HIC profile (HIC DAR 7.7) is illustrated in FIG. 66.

ADC2-64-2 SEC profile with >99% purity is illustrated in FIG. 67.

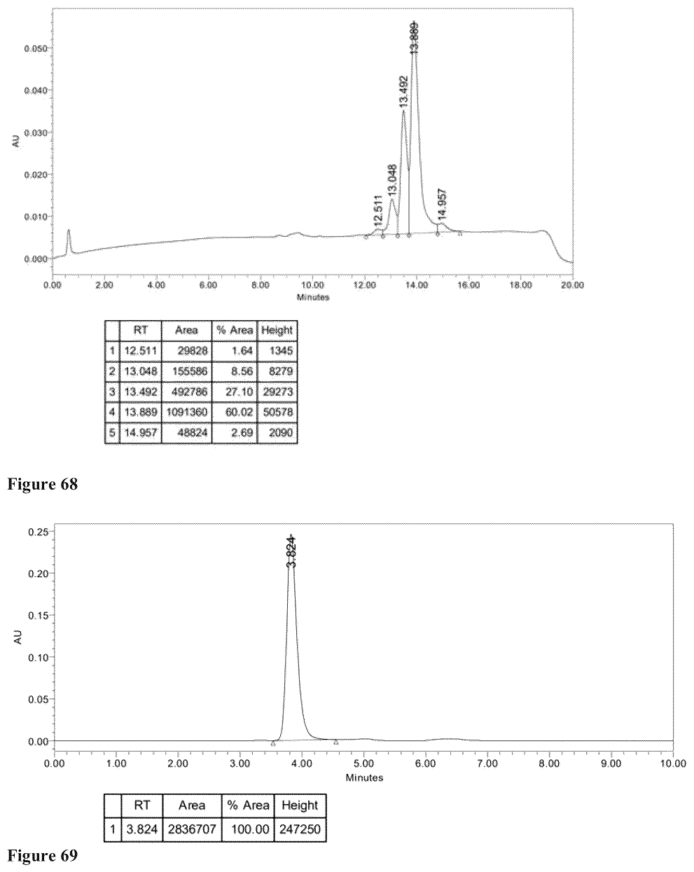

ADC2-65-2 HIC profile (HIC DAR 7.5) is illustrated in FIG. 68.

ADC2-65-2 SEC profile with >99% purity is illustrated in FIG. 69.

### Cathepsin B Mediated Release

### Method

### Cathepsin B Dilution Preparation

CTSB was activated by incubation at ambient temperature for 15 min with 30 mM dithiothreitol and 15 mM EDTA at PH5.5.

Reaction buffer preparation: 25 mM sodium acetate & 1 mM EDTA, PH5.5.

Adding 150 nM NAcCys-13 & NAcCys-14 & NAcCys-15 & NAcCys-16 & NAcCys-17 and 0.3 unit activated cathepsin B mixture according to the experiment template.

The reaction mixtures were incubated at 37° C. immediately in the reader, then start reading using the kinetic set-up described. (TECAN SPARK: excitation/emission: 485/520 nm) (120 cycles, every one min) Gain Manual: 77%

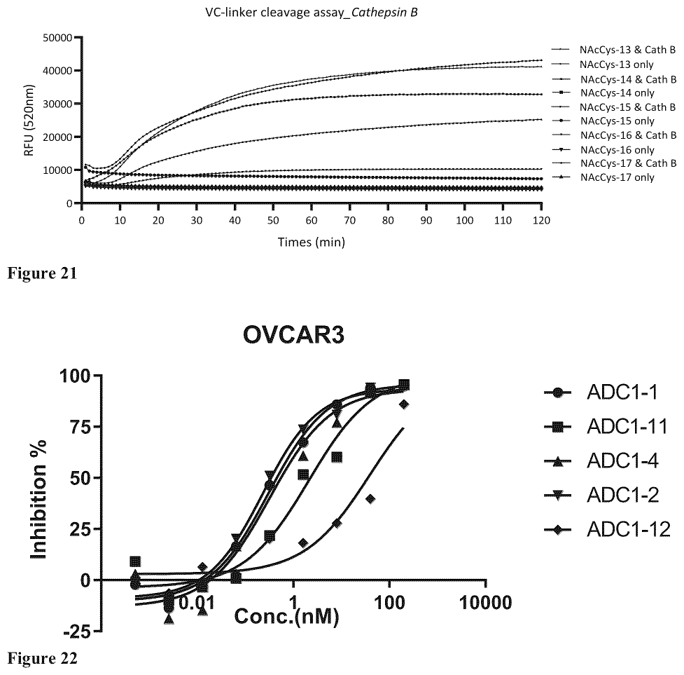

Data analysis by GraphPad Prism 9 is illustrated in FIG. 21.

### Cellular Killing on PTK7 Cellular Lines

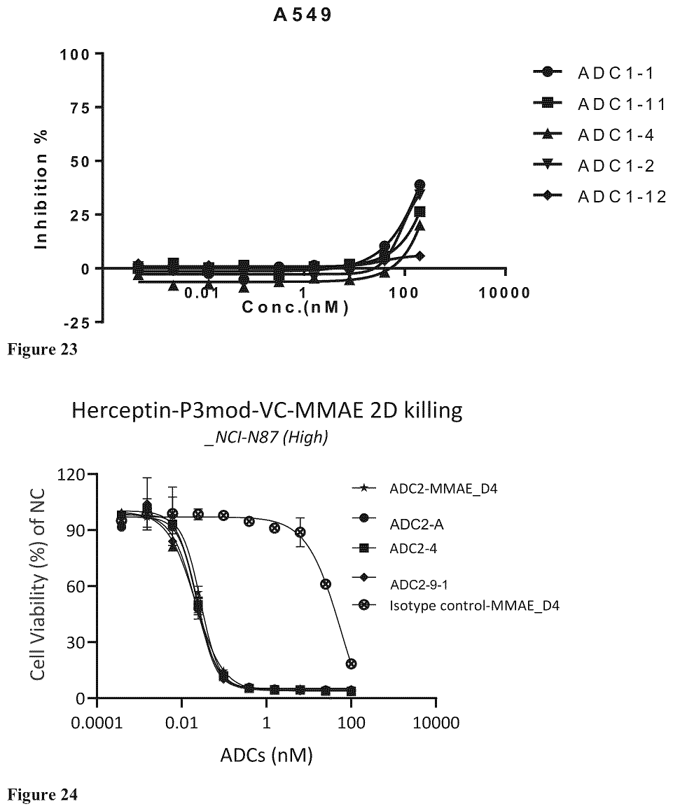

Cells were seeded into a 96-well plate at a density of 1×103 per well in RPMI-1640 medium containing 10% FBS. Cells were then treated with a 10-points titration of compounds (nine concentration groups: 0.000512, 0.00256, 0.0128, 0.064, 0.32, 1.6, 8, 40, 200 nM and an empty control group with medium containing 0.1% DMSO), including ADC1-1, ADC1-11, ADC1-4, ADC1-2 and ADC1-12. The plates were incubated for 7 days at 37° C., 5% CO2. Next, 50 μL of Cell-Titer-Glo reagent was added to each well. The mixture was mixed on an orbital shaker for 10 mins to allow cell lysis. Then, the luminescent signal was measured using BMG PheraStar with the luminescent protocol. The inhibition of Ovcar3 and A549 cell lines is illustrated in FIG. 22 and FIG. 23, respectively. The data for the inhibition of Ovcar3 and A549 is provided in Table 5.

TABLE 5

| | Anti-proliferation | | | |
|---|---|---|---|---|
| | Ovcar3 (PTK7 high) | | A549 (PTK7 low) | |
| Sample ID | IC50 (nM) | Emax (%) | IC50 (nM) | Emax (%) |
| ADC1-1 | 0.31 | 95.76 | >200 | 38.87 |
| ADC1-11 | 2.1 | 100.9 | >200 | 26.44 |
| ADC1-4 | 0.32 | 93.45 | >200 | 20.12 |
| ADC1-2 | 0.23 | 93.47 | >200 | 34.22 |
| ADC1-12 | 39.39 | 86.04 | >200 | 5.76 |

### ADCs-MMAE (DAR4/DAR8) Cellular Killing on HER2 Cell Lines

### Method

Seeding cells (NCI-N87) into 96-well plate (Greiner: 655090), at 2E3 cell per well (100 ul/well). Overnight incubation.

Adding the fresh growth-medium containing the varying concentrations (from 100 nM. 4 folds dilution) of ADCs, 50 ul/well.

Incubate at 37° C., 6 days.

The cell viability was detected by Cell Titer-Glo (Promega, G7573). Add 70 L/well of Cell Titer-Glo Reagent in each well.

Allow the plate to incubate at room temperature for 10 minutes to stabilize the luminescent signal.

The samples were analyzed with Microplate Reader.

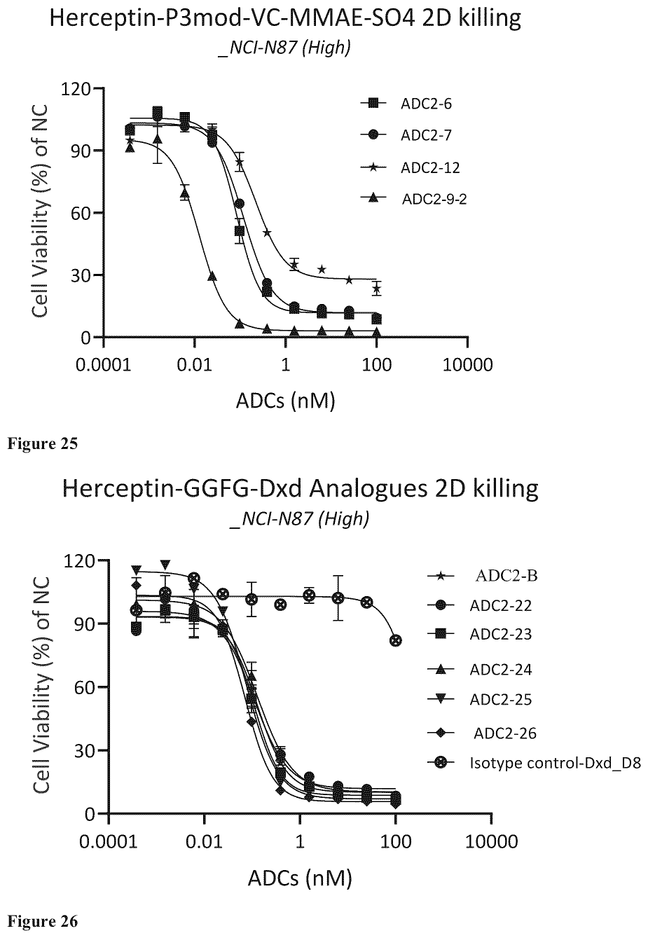

The data of the NCI-N87 cell viability detection is illustrated in FIG. 24 and FIG. 25. The data for the NCI-N87 cell viability detection is provided in Table 6.

TABLE 6

| | NCI-N87 cell viability detection | |
|---|---|---|
| ADC examples | Emax (%) | EC50 (nM) |
| ADC2-A | 95.3 | 0.028 |
| ADC2-2 | 95.7 | 0.023 |
| ADC2-4 | 96.3 | 0.024 |
| ADC2-1 | 96 | 0.021 |
| ADC2-9-1 | 96.5 | 0.021 |
| ADC2-6 | 91.3 | 0.095 |
| ADC2-7 | 90.7 | 0.131 |
| ADC2-12 | 76.5 | 0.256 |
| ADC2-9-2 | 97.4 | 0.012 |

### ADCs-Dxd Analogues (DAR8) Cellular Killing

### Method

Seed cells (NCI-N87) into 96-well plate (Greiner: 655090), at 2E3 cell per well (100 ul/well). Overnight incubation. Add the fresh growth-medium containing the varying concentrations (from 100 nM. 4 folds dilution) of ADCs, 50 ul/well.

Incubate at 37° C., 6 days.

The cell viability was detected by Cell Titer-Glo (Promega, G7573). Add 70 L/well of Cell Titer-Glo Reagent in each well.

Allow the plate to incubate at room temperature for 10 minutes to stabilize the luminescent signal.

Analyze with Microplate Reader. The data for the NCI-N87 cell viability detection is provided in Table 7. The data for the NCI-N87 cell viability detection is illustrated in FIG. 26.

TABLE 7

| | NCI-N87 cell viability detection | |
|---|---|---|
| Sample ID | Emax (%) | EC50 (nM) |
| ADC2-B | 92.3 | 0.118 |
| ADC2-22 | 91.5 | 0.110 |
| ADC2-23 | 93.3 | 0.104 |
| ADC2-24 | 91.5 | 0.139 |
| ADC2-25 | 93.6 | 0.095 |
| ADC2-26 | 95.5 | 0.076 |

### ADCs-P5mod-GGFG-Dxd (DAR8) Cellular Killing

### Method

Seeding cells (NCI-N87 & MDA-MB-468) into 96-well plate (Greiner: 655090), at 2E3 cell per well (100 ul/well). Overnight incubation.

Adding the fresh growth-medium containing the varying concentrations (from 100 nM. 4 folds dilution) of ADCs, 50 ul/well.

Incubate at 37° C., 6 days.

The cell viability was detected by Cell Titer-Glo (Promega, G7573). Add 70 L/well of Cell Titer-Glo Reagent in each well.

Allow the plate to incubate at room temperature for 10 minutes to stabilize the luminescent signal.

Analyze with Microplate Reader.

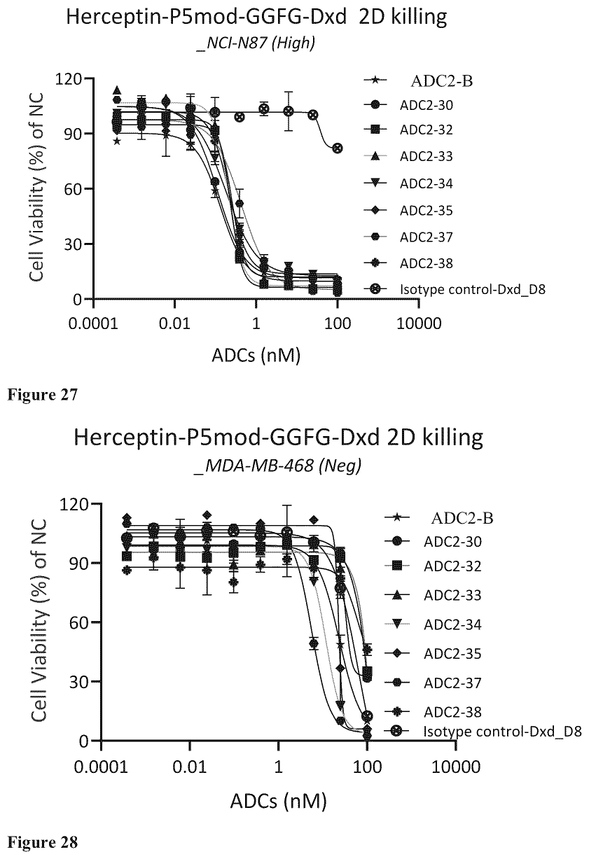

The data for the NCI-N87 cell viability detection is provided in Table 8. The data for the NCI-N87 cell viability detection is illustrated in FIG. 27 and FIG. 28.

TABLE 8

| | NCI-N87 cell viability detection | |
|---|---|---|
| Sample ID | Emax (%) | EC50 (nM) |
| ADC2-B | 92.3 | 0.118 |
| ADC2-30 | 91.8 | 0.130 |
| ADC2-32 | 95.2 | 0.234 |
| ADC2-33 | 94.2 | 0.262 |
| ADC2-34 | 90.6 | 0.211 |
| ADC2-35 | 91.2 | 0.237 |
| ADC2-37 | 96.8 | 0.434 |
| ADC2-38 | 89.1 | 0.229 |
| ADC2-51 | 83.2 | 0.194 |
| ADC2-54 | 86.5 | 0.143 |

### ADCs-P5mod-GGFG-Dxd (DAR8) (RO) Cellular Killing

### Method

Seed cells (NCI-N87 & MDA-MB-468) into 96-well plate (Greiner: 655090), at 2E3 cell per well (100 ul/well). Overnight incubation.

Add the fresh growth-medium containing the varying concentrations (from 100 nM. 4 folds dilution) of ADCs, 50 ul/well.

Incubate at 37° C., 6 days.

The cell viability was detected by Cell Titer-Glo (Promega, G7573). Add 70 L/well of Cell Titer-Glo Reagent in each well.

Allow the plate to incubate at room temperature for 10 minutes to stabilize the luminescent signal.

Analyze with Microplate Reader.

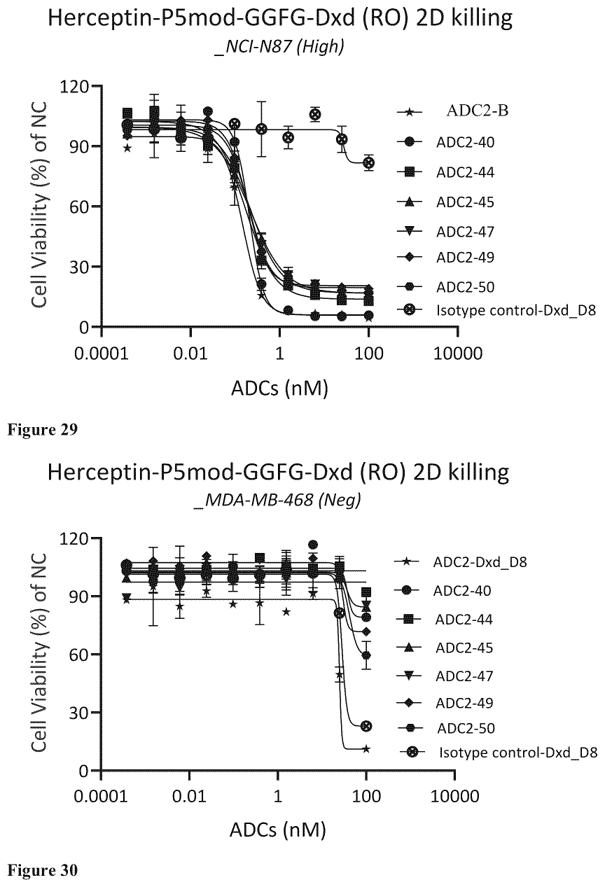

The data for the NCI-N87 cell viability detection is provided in Table 9. The data for the NCI-N87 cell viability detection is illustrated in FIG. 29 and FIG. 30.

TABLE 9

| | NCI-N87 cell viability | |
|---|---|---|
| | detection | |
| Sample ID | Eₘₐₓ (%) | EC₅₀ (nM) |
| ADC2-8 | 92.3 | 0.118 |
| ADC2-40 | 94.2 | 0.216 |
| ADC2-44 | 87.1 | 0.186 |
| ADC2-45 | 85.8 | 0.228 |
| ADC2-46 | 85.6 | 0.233 |
| ADC2-48 | 87.8 | 0.111 |
| ADC2-49 | 81.1 | 0.2 |
| ADC2-50 | 83.0 | 0.209 |

### ADCs-P3mod-VC/VA-Aminal-Dxd (DAR8) Cellular Killing

### Method

Seeding cells (NCI-N87 & MDA-MB-468) into 96-well plate (Greiner: 655090), at 2E3 cell per well (100 ul/well). Overnight incubation.

Adding the fresh growth-medium containing the varying concentrations (from 100 nM. 4 folds dilution) of ADCs, 50 ul/well.

Incubate at 37° C., 6 days.

The cell viability was detected by Cell Titer-Glo (Promega, G7573). Add 70 L/well of Cell Titer-Glo Reagent in each well.

Allow the plate to incubate at room temperature for 10 minutes to stabilize the luminescent signal.

Analyze with Microplate Reader.

The data for the NCI-N87 cell viability detection is provided in

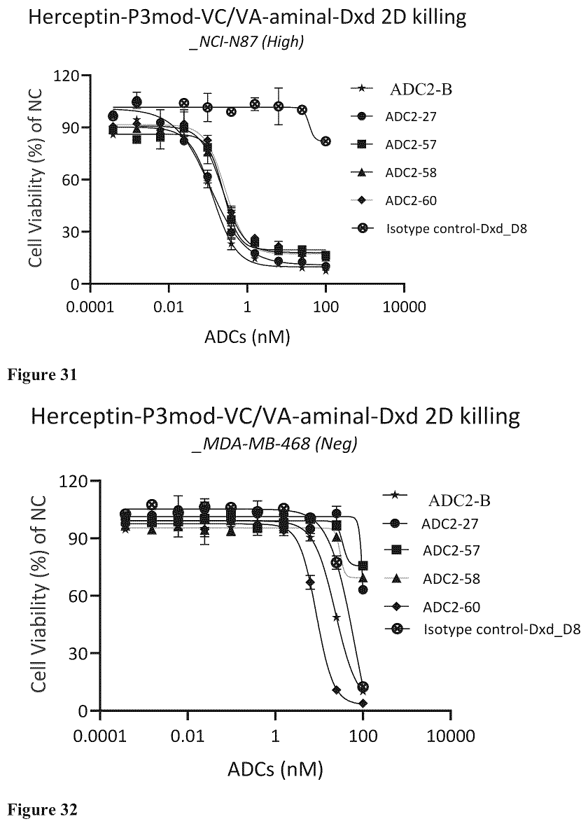

Table 10 The data for the NCI-N87 cell viability detection is illustrated in FIG. 31 and FIG. 32.

TABLE 10

| | NCI-N87 cell viability | |
|---|---|---|
| | detection | |
| Sample ID | Eₘₐₓ (%) | EC₅₀ (nM) |
| ADC2-B | 92.3 | 0.118 |
| ADC2-27 | 89.8 | 0.116 |
| ADC2-57 | 83.6 | 0.213 |
| ADC2-58 | 84.7 | 0.212 |
| ADC2-59 | 88.1 | 0.12 |
| ADC2-60 | 90.3 | 0.271 |
| ADC2-61 | 87.9 | 0.173 |

### ADCs-P5mod-GGFG-Aminal-Dxd (DAR8) Cellular Killing

Seed cells (HCC1569) into 96-well plate (Corning 3D plates or Sarstedt plate), at 2E3 cell per well (80 ul/well). Incubate overnight.

Add the fresh growth-medium containing the varying concentrations (from 100 nM. 4 folds dilution) of ADCs, 40 ul/well.

Incubate at 37° C. for 6 days.

The cell viability was detected by 3D reagent (Promega, G9683). Add 100 μL/well of the 3D reagent in each well.

Allow the plate to incubate at room temperature for 30 minutes to stabilize the luminescent signal.

Analyze with Microplate Reader.

ADCs (P5mod-GGFG-aminal-Dxd ADCs) have the similar killing efficiency to ref ADC (ADC3-1) as provided in Table 11 and Table 12, and FIGS. 38 (A) and (B).

TABLE 11
HCC1569 cell killing

| Name | Eₘₐₓ (%) | EC₅₀ (nM) |
|---|---|---|
| ADC3-1 | 78.6 | 0.682 |
| ADC3-2 | 79.1 | 0.517 |
| ADC3-6 | 78.2 | 0.315 |
| ADC3-3 | 79 | 0.495 |
| ADC3-4 | 74.4 | 0.681 |

TABLE 12
HCC1569 cell killing

| Name | Eₘₐₓ (%) | EC₅₀ (nM) |
|---|---|---|
| ADC3-1 | 66.4 | 3.148 |
| ADC3-9 | 65.1 | 3.184 |
| ADC3-8 | 66.1 | 3.171 |
| ADC3-7 | 68.9 | 3.446 |
| ADC3-10 | 64.7 | 4.791 |
| ADC3-5 | 62.8 | 4.361 |

### ADCs-P3mod-VA/VA-Aminal-Dxd (DAR8) Cellular Killing

Seeding cells (HCC1569) into 96-well plate (Corning 3D plates or Sarstedt plate), at 2E3 cell per well (80 ul/well). Overnight incubation.

Adding the fresh growth-medium containing the varying concentrations (from 100 nM. 4 folds dilution) of ADCs, 40 ul/well.

Incubate at 37° C. for 6 days.

The cell viability was detected by 3D reagent (Promega, G9683). Add 100 L/well of the 3D reagent in each well.

Allow the plate to incubate at room temperature for 30 minutes to stabilize the luminescent signal.

Analyze with Microplate Reader.

ADCs (P3mod-VC/VA-aminal-Dxd ADCs) have the similar killing efficiency to ref ADC (ADC3-1) as provided in Table 13 and Table 14, and FIGS. 39 (A) and (B).

TABLE 13
HCC1569 cell killing

| Name | Emax (%) | EC50 (nM) |
|---|---|---|
| ADC3-1 | 77.1 | 0.453 |
| ADC3-15 | 77.7 | 0.365 |
| ADC3-12 | 75.6 | 0.531 |

TABLE 14
HCC1569 cell killing

| Name | Emax (%) | EC50 (nM) |
|---|---|---|
| ADC3-1 | 66.4 | 3.148 |
| ADC3-11 | 67.9 | 2.233 |
| ADC3-14 | 74.1 | 1.286 |

### MMAE Base ADC Cellular Assay

### Method

Seed cells (JIMT-1, 2E3 cells/well or Capan-1, 4E3 cells/well) into 96-well plate (Greiner: #655090), 100 ul/well. Overnight incubation. Add the fresh growth-medium containing the varying concentrations (from 100 nM. 4 folds dilution) of ADCs, 50 ul/well. Incubate at 37° C., 6 days. The cell viability was detected by Cell-titer Glo (Promega, G7573). Add 70 L/well of the reagent in each well. Allow the plate to incubate at room temperature for 10 minutes to stabilize the luminescent signal. Analyze with Microplate Reader.

### Results

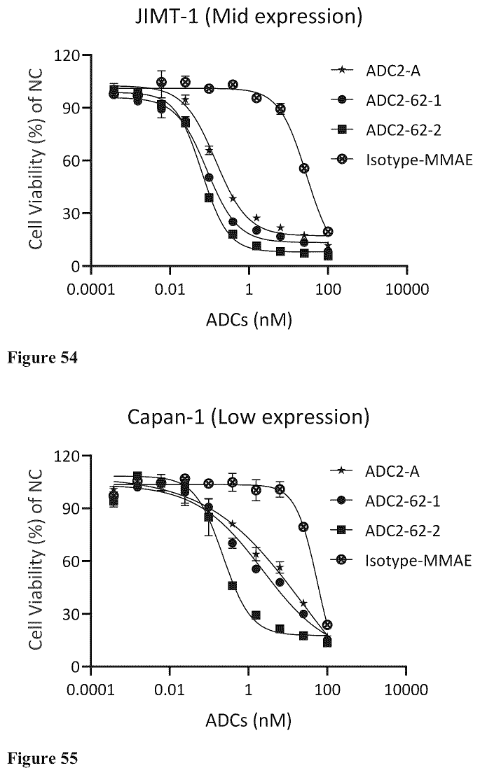

It is surprising and unexpected that ADCs have comparable cell-killing activity to the reference ADC (ADC2-A) in JIMT-1 (HER2 moderate expression level), and ADC2-62-2(DAR8) even has better cell-killing activity compared to the reference ADC in Capan-1(HER2 low expression level). The data is provided in Table 15, Table 16, FIG. 54 and FIG. 55.

TABLE 15
JIMT-1 (Mid expression) cell killing

| Name | Emax (%) | EC50 (nM) |
|---|---|---|
| ADC2-A | 88.4 | 0.172 |
| ADC2-62-1 | 91.7 | 0.088 |
| ADC2-62-2 | 94.2 | 0.065 |

TABLE 16
Capan-1 (Low expression) cell killing

| Name | Emax (%) | EC50 (nM) |
|---|---|---|
| ADC2-A | 83.1 | 3.936 |
| ADC2-62-1 | 84.8 | 1.84 |
| ADC2-62-2 | 86.3 | 0.285 |

### Method

Seed cells (JIMT-1, 2E3 cells/well or Capan-1, 4E3 cells/well) into 96-well plate (Greiner: #655090), 100 ul/well. Overnight incubation. Add the fresh growth-medium containing the varying concentrations (from 100 nM. 4 folds dilution) of ADCs, 50 ul/well. Incubate at 37° C. for 6 days. The cell viability was detected by Cell-titer Glo (Promega, G7573). Add 70 L/well of the reagent in each well. Allow the plate to incubate at room temperature for 10 minutes to stabilize the luminescent signal. Analyze with Microplate Reader.

### Results

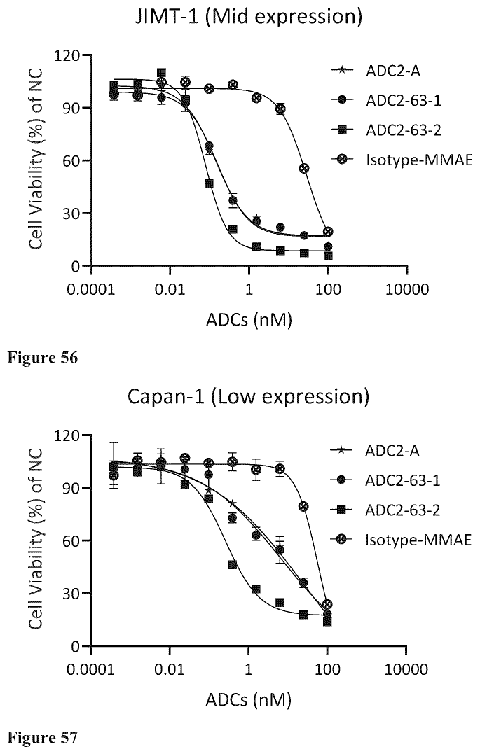

ADC2-63-1 (DAR4) has comparable cell-killing activity compared to the reference ADC (ADC2-A with DAR4) in JIMT-1 (HER2 moderate expression level) and Capan-1 (HER2 low expression level). ADC2-63-2 (DAR8) has a better cell-killing activity to the reference ADC in JIMT-1 (HER2 moderate expression level) and Capan-1(HER2 low expression level) as provided in Table 17, Table 18 and FIG. 56, and FIG. 57.

TABLE 17
JIMT-1 (Mid expression) cell killing

| Name | Emax (%) | EC50 (nM) |
|---|---|---|
| ADC2-A | 88.4 | 0.172 |
| ADC2-63-1 | 88.9 | 0.178 |
| ADC2-63-2 | 94.4 | 0.09 |

TABLE 18
Capan-1 (Low expression) cell killing

| Name | Emax (%) | EC50 (nM) |
|---|---|---|
| ADC2-A | 83.1 | 3.936 |
| ADC2-63-1 | 81.7 | 3.074 |
| ADC2-63-2 | 86.1 | 0.3 |

### Method

Seed cells (JIMT-1, 2E3 cells/well or Capan-1, 4E3 cells/well) into 96-well plate (Greiner: #655090), 100 ul/well. Incubate overnight. Add the fresh growth-medium containing the varying concentrations (from 100 nM. 4 folds dilution) of ADCs at 50 ul/well. Incubate at 37° C. for 6 days. The cell viability was detected by Cell-titer Glo (Promega, G7573). Add 70 μL/well of the reagent in each well. Allow the plate to incubate at room temperature for 10 minutes to stabilize the luminescent signal. Analyze with Microplate Reader.

### Results

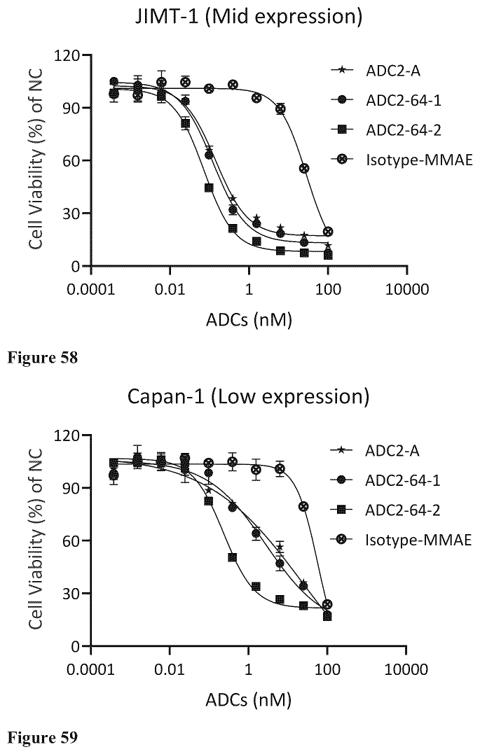

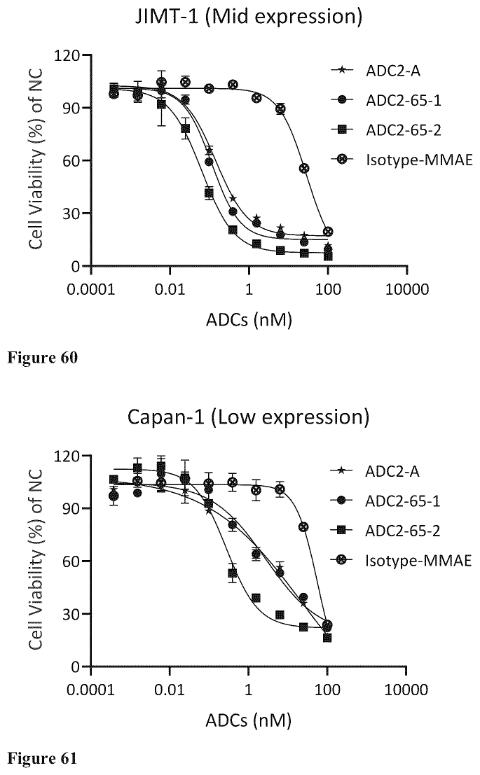

ADC2-64-1(DAR4) has comparable cell-killing activity to the reference ADC (ADC2-A with DAR4) in JIMT-1 (HER2 moderate expression level) and Capan-1(HER2 low expression level). ADC2-64-2 with DAR8 has better cell-killing activity than the reference ADC (ADC2-A with DAR4) in JIMT-1 (HER2 moderate expression level) and Capan-1(HER2 low expression level) as provided in Table 19, Table 20, FIG. 58, and FIG. 59.

TABLE 19
JIMT-1 (Mid expression) cell killing

| Name | Emax (%) | EC50 (nM) |
|---|---|---|
| ADC2-A | 88.4 | 0.172 |
| ADC2-64-1 | 92.5 | 0.153 |
| ADC2-64-2 | 94 | 0.076 |

TABLE 20
Capan-1 (Low expression) cell killing

| Name | Emax (%) | EC50 (nM) |
|---|---|---|
| ADC2-A | 83.1 | 3.936 |
| ADC2-64-1 | 82.1 | 2.56 |
| ADC2-64-2 | 83.2 | 0.302 |

### ADC Plasma Stability Evaluation

Incubation of ADC with Plasma

Dilute ADC into mouse or human plasma to yield a final solution of 100 g/mL ADC in plasma. Incubate the samples at 37° C. Aliquots (100 L) were taken at four time points (0, 4, 24, 72, 96, or 168 h). Samples were frozen at −80° C. until analysis. Plasma Payload concentrations were carried out under the following measurement conditions:
Instrument: LC-MS/MS (Triple Quad 5500)
Monitor: MRM
Column: Advanced Materials Technology, HALO AQ-C18 2.7 μm 90 Å, 50*2.1 mm
Column temperature: 40° C.
Mobile phase A: H2O-0.1% FA
Mobile phase B: ACN-0.11% FA
Gradient program for MMAE: 20% B-20% B (0 min-0.2 min), 20% B-80% B (0.2 min-1.5 min), 80% B-80% B (1.5 min-2.2 min), 80% B-20% B (2.20 min-2.21 min), 20% B-20% B (2.21 min-3.0 min); Gradient program for Dxd: 2% B-2% B (0 min-0.2 min), 2% B-98% B (0.2 min-1.2 min), 98% B-98% B (1.2 min-2.0 min), 98% B-2% B (2.0 min-2.01 min), 2% B-2% B (2.01 min-4.0 min);
Injected sample amount: 2 L(MMAE), 10 L(DXd)
Plasma ADC and total Ab (Tab) concentrations were carried out under the following measurement conditions:
Assay: Ligand binding assay (ELISA)
Capture reagent: Her2ECD
Detection reagent: anti-payload Ab for ADC and anti-human IgG polyclonal Ab for Total Ab.

### ADC DAR Changes in Human/Mouse Plasma Stability Study Samples

### Method

Human Her2 extracellular domain (ECD) was biotinylated and immobilized onto Dynabeads M-280 Streptavidin, and then the ADCs were captured by ECD-bead system from plasma samples for 2 hours at room temperature. The captured ADCs were then washed with HBS-EP buffer (10 mM Hepes [pH 7.4], 150 mM NaCl, 3.4 mM ethylenediaminetetraacetic acid [EDTA], 0.005% Surfactant P20) and digested using IdeS enzyme at 37° C. for 1 h. After extensive washing of the beads with HBS-EP, water and 10% acetonitrile, the ADC analytes were eluted using 30% acetonitrile with 1% formic acid. Liquid chromatography-tandem mass spectrometry (LC-MS/MS) method were used for ADC DAR analysis.

### ADC Rat Pharmacokinetic Study

### Methods

All animal studies were carried out in accordance with the Institutional Animal Care and Use Committee (IACUC). 6-8 weeks old female BALB/c nude mice were purchased from Beijing Vital River Laboratory Animal Technology Co., Ltd., while 6 weeks old male Sprague Dawley rats with jugular vein catheter were purchased from SKILLS Model Animal Research Technology Co., Ltd. (Beijing, China). All animals were maintained under specific pathogen free (SPF) condition with free access to food and water. ADCs in 20 mM Histidine-acetate, pH 5.5 were administered intravenously into mice or rats at 3 mg/kg. Blood samples were collected at 0, 4, 8, 24, 48, 72, 168, 240, and 336 h after administration, followed by centrifugation (4° C., 3000×g, 7 min) to separate plasma.

The concentrations of ADCs and total Abs were measured by in-house Meso Scale Discovery (MSD) ligand binding methods. Briefly, his tagged Her2 extracellular domain fusion protein was used as a capture reagent, biotin labelled anti-DXd Ab, or anti-MMAE Ab, or goat anti-human kappa Ab were used as the detector reagents for Dxd based ADCs, or MMAE based ADCs, or total Ab assays, respectively. Liquid chromatography-tandem mass spectrometry (LC-MS/MS) method were used for quantification of DXd or MMAE.

Plasma ADC and total Ab(Tab) concentrations were carried out under the following measurement conditions:
Assay: Ligand binding assay (ELISA)
Capture reagent: Her2ECD
Detection reagent: anti-payload Ab for ADC and anti-human IgG polyclonal Ab for Total Ab.
ADC mouse pharmacokinetic study-pre-mature payload release study
Plasma Payload concentrations were carried out under the following measurement conditions:
(1) Instrument: LC-MS/MS (Triple Quad 5500)
(2) Monitor: MRM
(3) Column: Advanced Materials Technology, HALO AQ-C18 2.7 μm 90 Å, 50*2.1 mm
(4) Column temperature: 40° C.
(5) Mobile phase A: H2O-0.1% FA
(6) Mobile phase B: ACN-0.1% FA
(7) Gradient program: 20% B-20% B (0 min-0.2 min), 20% B-80% B (0.2 min-1.5 min), 80% B-80% B (1.5 min-2.2 min), 80% B-20% B (2.20 min-2.21 min), 20% B-20% B (2.21 min-3.0 min)
(8) Injected sample amount: 2 μL

ADC DAR changes in PK study sample.

Goat Anti-Human IgG/F(ab′)₂ fragment specific was biotinylated and immobilized onto Dynabeads M-280 Streptavidin, and then the ADCs were captured by ECD-bead system from plasma samples for 2 hours at room temperature. The captured ADCs were then washed with HBS-EP buffer (10 mM Hepes [pH 7.4], 150 mM NaCl, 3.4 mM ethylenediaminetetraacetic acid [EDTA], 0.005% Surfactant P20) and digested using IdeS enzyme at 37° C. for 1 h. After extensive washing of the beads with HBS-EP, water and 10% acetonitrile, the ADC analytes were eluted using 30% acetonitrile with 1% formic acid. Liquid chromatography-tandem mass spectrometry (LC-MS/MS) method were used for ADC DAR analysis.

### Efficacy Study

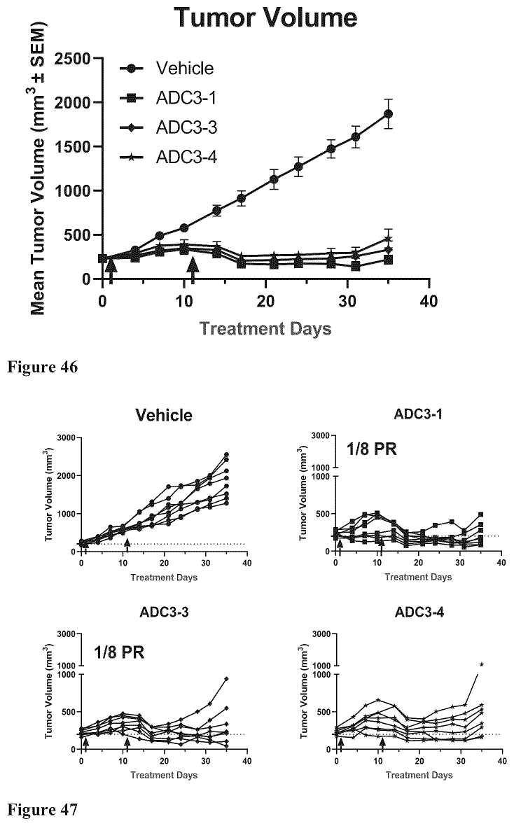

### Method for FIG. 46 and FIG. 47

Female BALB/c nude mice were subcutaneously implanted with 1×10⁷ MX-1 cells per 200 μL PBS/matrigel in the right flank. After inoculation, tumor volumes were determined twice weekly in two dimensions using a caliper, and were expressed in mm³ using the formula: V=0.5(a×b²) where a and b are the long and short diameters of the tumor, respectively. When tumors reached a mean volume of approximately 230 mm³ in size, mice were randomly allocated into 4 groups with 8 animals in each group, and were intravenously treated with vehicle, ADC3-1, ADC3-3, or ADC3-4 at 10 mg/kg on day 1 and day 11, respectively. Partial regression (PR) was defined as tumor volume smaller than 50% of the starting tumor volume on the first day of dosing in three consecutive measurements and complete regression (CR) was defined as tumor volume less than 14 mm³ in three consecutive measurements. Data is presented as mean tumor volume±standard error of the mean (SEM). Tumor growth inhibition (TGI) is calculated using formula (TGI):

$$\% growth inhibition = 100 \times {( 1 - ( \frac{( treated t ) - ( treated to )}{( placebo t ) - ( placebo to )} ) )}_{( TGI )}$$

treated t=treated tumor volume at time t
treated t₀=treated tumor volume at time 0
placebo t=placebo tumor volume at time t
placebo t₀=placebo tumor volume at time 0

### Results for FIG. 46 and FIG. 47

The in vivo efficacy of different ADCs was compared in MX-1 breast cancer xenografts grown subcutaneously in BALB/c nude mice. Results are shown in FIG. 47. Treatment with ADC3-1, ADC3-3, or ADC3-4 at 10 mg/kg resulted in 101%, 94%, or 86% tumor growth inhibition (TGI) on day 35, respectively (FIG. 47). For individual animal tumor growth inhibition, ADC3-3 also induced 1/8 PR, while ADC3-1 induced no PR (FIG. 47). Meanwhile all three ADCs were well tolerated without any sign of toxicity or significant body weight decreased. Taken together, ADC3-3 and ADC3-4 demonstrated comparable efficacy with ADC3-1.

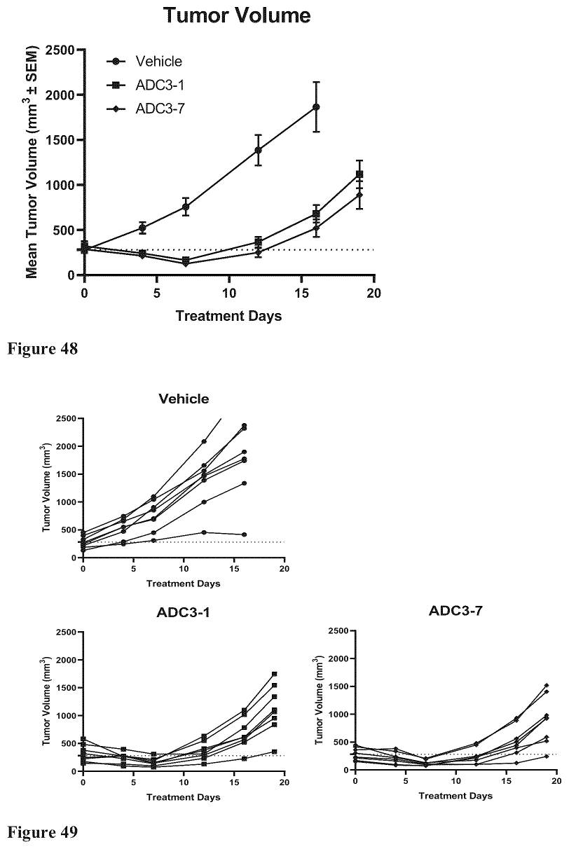

### Method for Efficacy Study for FIG. 48 and FIG. 49

Female BALB/c nude mice were subcutaneously implanted with 3×10⁶ A375 cells per 200 μL PBS in the right flank. When tumors reached a mean volume of approximately 280 mm³ in size, mice were randomly allocated into 3 groups with 8 animals in each group, and were intravenously treated with vehicle, ADC3-1, or ADC3-7 at 10 mg/kg on day 1, respectively.

### Results for FIG. 48 and FIG. 49

The in vivo efficacy of different ADCs was compared in A375 melanoma xenografts grown subcutaneously in BALB/c nude mice. Treatment with ADC3-1 or ADC3-7 at 10 mg/kg resulted in 77%, or 85% TGI on day 16, respectively (FIG. 49). ADC3-7 demonstrated superior efficacy than ADC3-1 in A375 xenograft model. Both ADCs were well tolerated without any sign of toxicity or significant body weight decreased.

### Method for Efficacy Study for FIGS. 61 and 62

Female BALB/c nude mice were subcutaneously implanted with 3×10⁶ A375 cells per 200 μL PBS in the right flank. When tumors reached a mean volume of approximately 280 mm³ in size, mice were randomly allocated into 4 groups with 8 animals in each group, and were intravenously treated with vehicle, ADC3-1, ADC3-12, or ADC3-15 at 10 mg/kg on day 1, respectively.

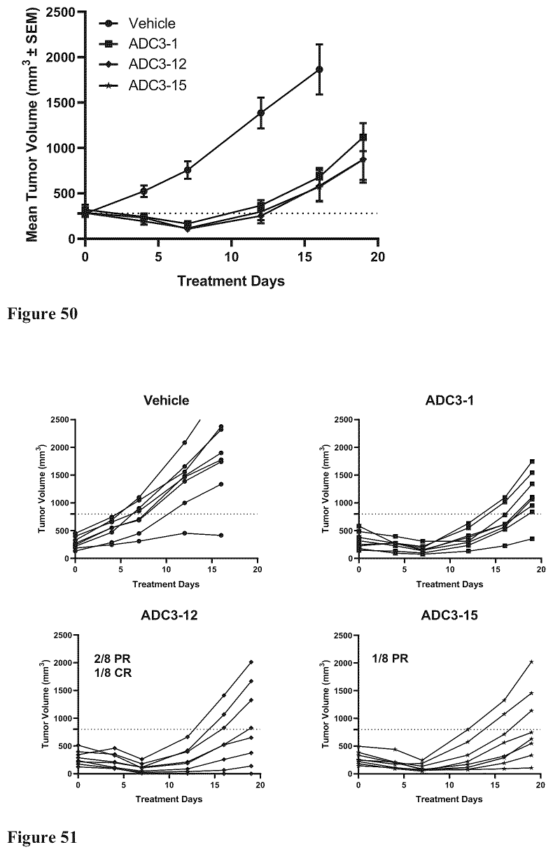

### Results for FIG. 50 and FIG. 51

The in vivo efficacy of different ADCs was compared in A375 melanoma xenografts grown subcutaneously in BALB/c nude mice. Results are shown in FIG. 8. Treatment with ADC3-1, ADC3-12, or ADC3-15 at 10 mg/kg resulted in 77%, 81%, or 82% TGI on day 16, respectively (FIG. 8A). For individual animal tumor growth inhibition, ADC3-12 also induced 2/8 PR and 1/8 CR, and ADC3-15 induced 1/8 PR, while ADC3-1 induced no PR (FIG. 8B). Taken together, both ADC3-12 and ADC3-15 demonstrated better anti-tumor efficacy than ADC3-1 in A375 xenograft model. Besides, all ADCs were well tolerated without any sign of toxicity or significant body weight decreased.

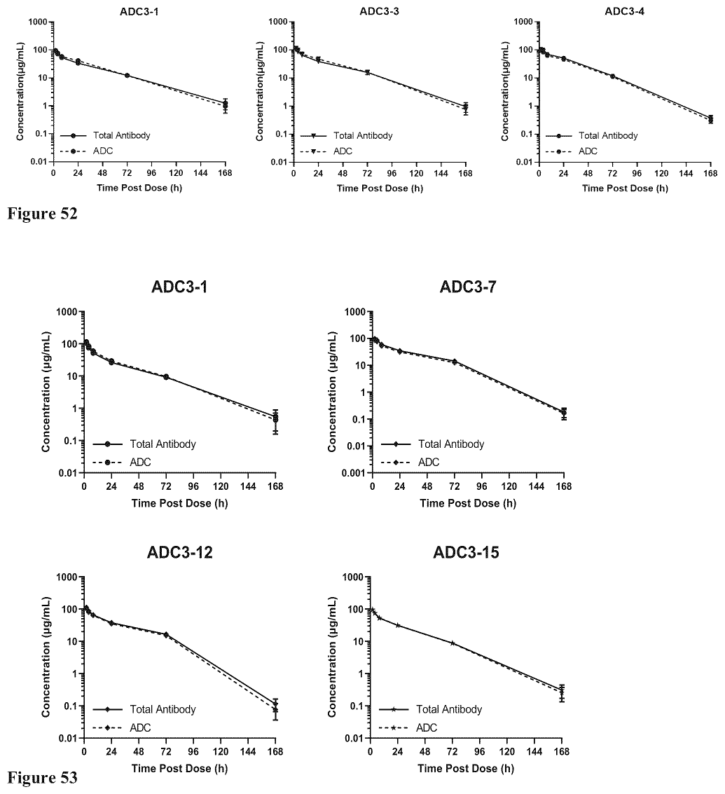

### PK Study Method for FIG. 52

Blood samples were collected at 0, 2, 4, 8, 24, 72, and 168 h after 10 mg/kg intravenously administration of ADC3-1, ADC3-3, or ADC3-4 from MX-1 tumor bearing mice, followed by centrifugation (4° C., 3000×g, 7 min) to separate plasma. The concentrations of ADCs and total Abs were measured by in-house developed Meso Scale Discovery (MSD) ligand binding methods. Briefly, his tagged Her3 extracellular domain fusion protein was used as a capture reagent, biotin labelled anti-DXd Ab, or goat anti-human kappa Ab were used as the detection reagents for ADCs or total Ab measurement, respectively.

### Result for FIG. 52

Overlapped ADC and total Ab concentration curves suggest very good linker stability of those three ADCs in mouse plasma. ADC3-3 and ADC3-4 exhibited comparable ADC or total Ab exposure and clearance with ADC3-1.

### PK Study Method for FIG. 53

Plasma samples were collected at 0, 2, 4, 8, 24, 72, and 168 h after 10 mg/kg intravenously administration of ADC3-1, ADC3-7, ADC3-12, or ADC3-15 from A375 tumor bearing mice. The concentrations of ADCs and total Abs were measured by in-house developed Meso Scale Discovery (MSD) ligand binding methods as described.

### Results for FIG. 53

Overlapped ADC and total Ab concentration curves suggest very good linker stability of those three ADCs in mouse plasma. ADC3-7, ADC3-12, and ADC3-15 exhibited comparable ADC or total Ab exposure and clearance with ADC3-1.

### Efficacy Test Method and ADC PK Study

### Method—JIMT-1

Female NOD.SCID mice are subcutaneously implanted with 3×10⁶ JIMT-1 cells per 200 μL PBS/matrigel in the right flank.

### Method—Capan-1

Female BALB/c Nude mice are subcutaneously implanted with 1×10⁷ Capan-1 cells per 200 μL PBS/matrigel in the right flank.

Method—PK Study

Serum samples are collected at 0, 2, 4, 8, 24, 72, and 168 h after intravenously administration. The concentrations of ADCs and total Abs are measured by in-house developed Meso Scale Discovery (MSD) ligand binding methods as described. Briefly, his tagged Her2 extracellular domain fusion protein is used as a capture reagent, biotin labelled anti-DXd Ab, or anti-MMAE Ab, or goat anti-human kappa Ab are used as the detector reagents for Dxd based ADCs, or MMAE based ADCs, or total Ab assays, respectively.

### Method CFPAC-1

Female BALB/c Nude mice are subcutaneously implanted with 1×10⁷ CFPAC-1 cells per 200 μL PBS/matrigel in the right flank. After inoculation, tumor volumes are determined twice weekly in two dimensions using a caliper, and are expressed in mm³ using the formula: V=0.5(a×b²) where a and b are the long and short diameters of the tumor, respectively.

When tumors reaches a mean volume of approximately 250 mm³ in size (After 7 days inoculation), mice are randomly allocated into groups, and are intravenously treated with ADCs at 10 mg/kg, respectively. Partial regression (PR) is defined as tumor volume smaller than 50% of the starting tumor volume on the first day of dosing in three consecutive measurements and complete regression (CR) is defined as tumor volume less than 14 mm³ in three consecutive measurements. Data is presented as mean tumor volume±standard error of the mean (SEM). Tumor growth inhibition (TGI) is calculated using formula (TGI):

FIG. 1 describes ADC2-B T/T0 and Payload Release % of ADC2-B in human plasma

FIG. 2 describes T/T0 and Payload Release % of ADC2-40 in human plasma

FIG. 3 describes T/T0 and Payload Release % of ADC2-32 in human plasma

FIG. 4 describes ADC2-B T/T0 and Payload Release % of ADC2-42 in human plasma

FIG. 6 describes Total Ab Rat PK Profiles after iv 3 mpk ADCs.

FIG. 7 describes Conjugated Ab Rat PK Profiles after iv 3 mpk ADCs.

FIG. 8 describes Conjugated Ab and Total Ab Rat PK Profiles after iv 3 mpk ADC2-B (free payload concentrations in all PK samples are below detection limit).

FIG. 10 describes Conjugated Ab and Total Ab Rat PK Profiles after iv 3 mpk ADC2-32 (free payload concentrations in all PK samples are below detection limit).

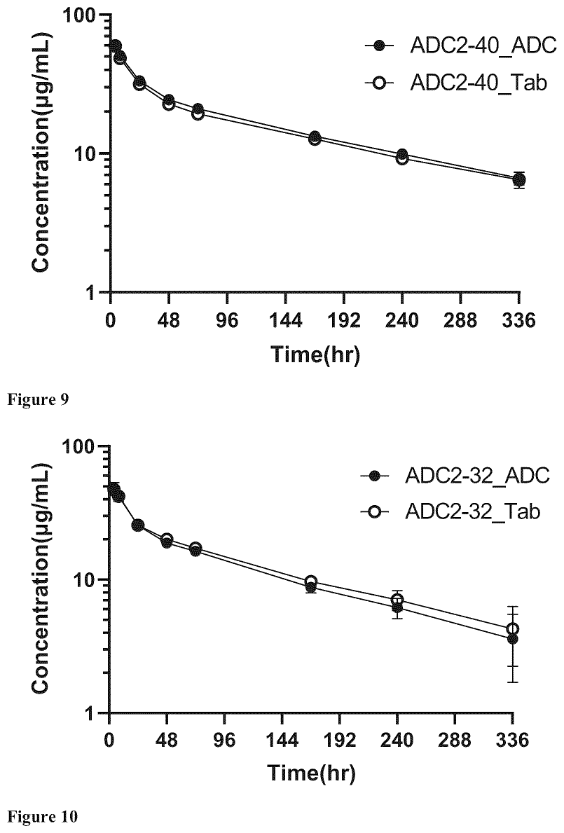

FIG. 9 describes Conjugated Ab and Total Ab Rat PK Profiles after iv 3 mpk ADC2-40 (free payload concentrations in all PK samples are below detection limit).

FIG. 10 describes Conjugated Ab and Total Ab Rat PK Profiles after iv 3 mpk ADC2-32 (free payload concentrations in all PK samples are below detection limit).

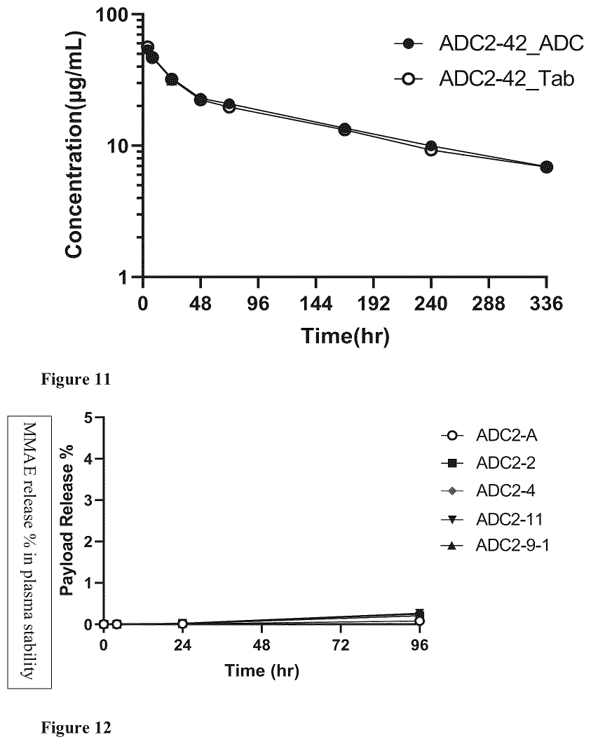

FIG. 11 describes Conjugated Ab and Total Ab Rat PK Profiles after iv 3 mpk ADC2-42 (free payload concentrations in all PK samples are below detection limit).

FIG. 12 describes Payload release % of ADCs in human plasma

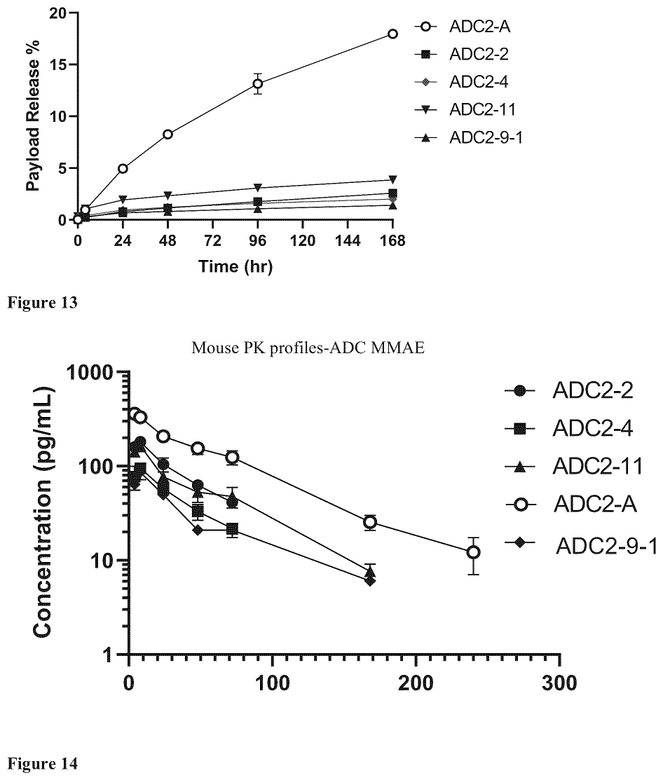

FIG. 13 describes Payload release % of ADCs in mouse plasma

FIG. 14 describes Free MMAE release in Mouse PK study after iv 3 mpk ADC.

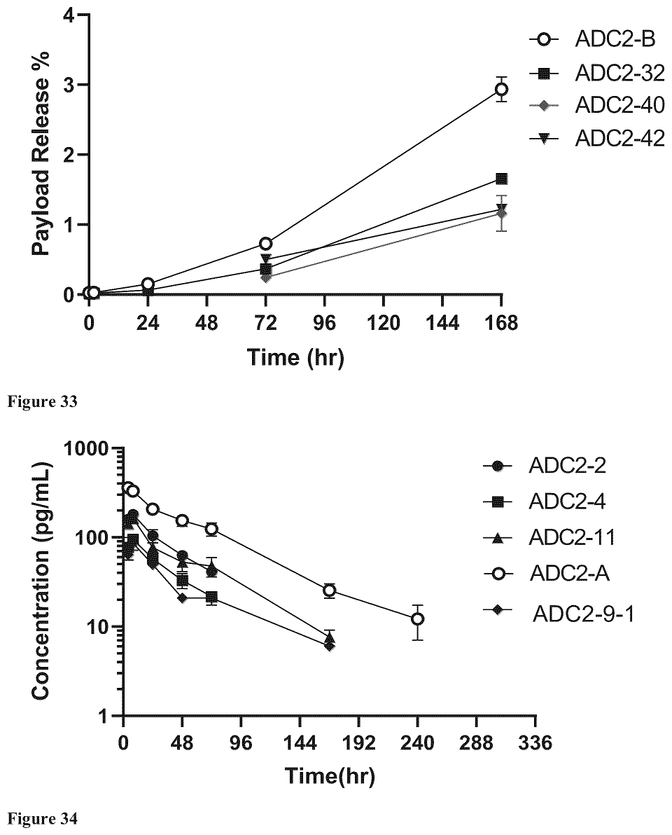

FIG. 33 describes Payload release % of ADCs in human plasma

FIG. 34 describes Free MMAE release in Mouse PK study.

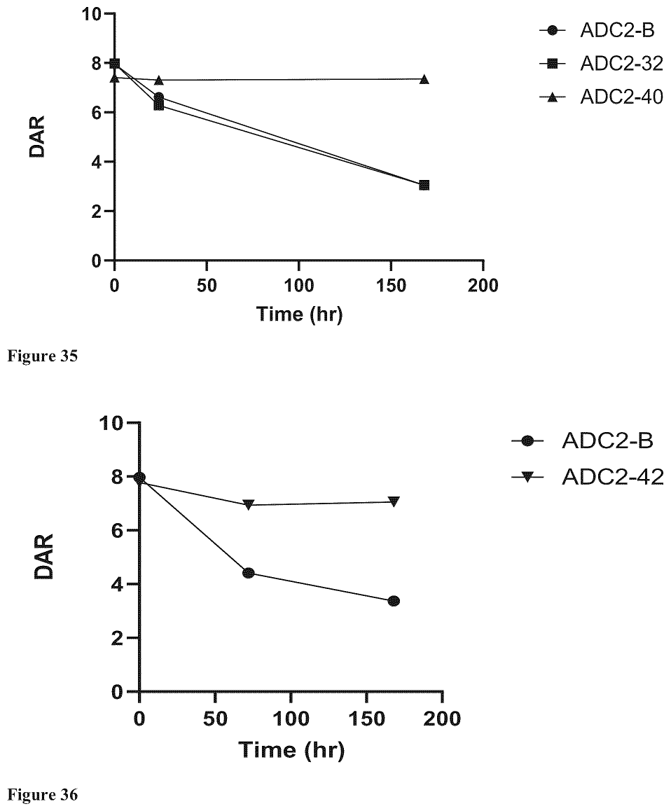

FIG. 35 illustrates ADC DAR changes of ADC2-B, ADC2-32, and ADC2-42 in human plasma.

FIG. 36 illustrates ADC DAR changes of ADC2-B and ADC2-42 in human plasma.

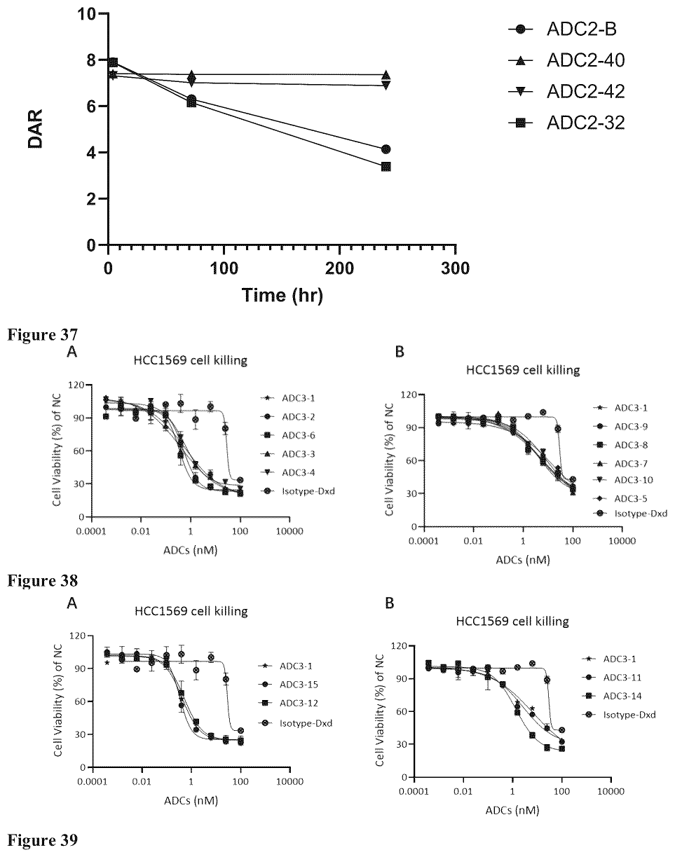

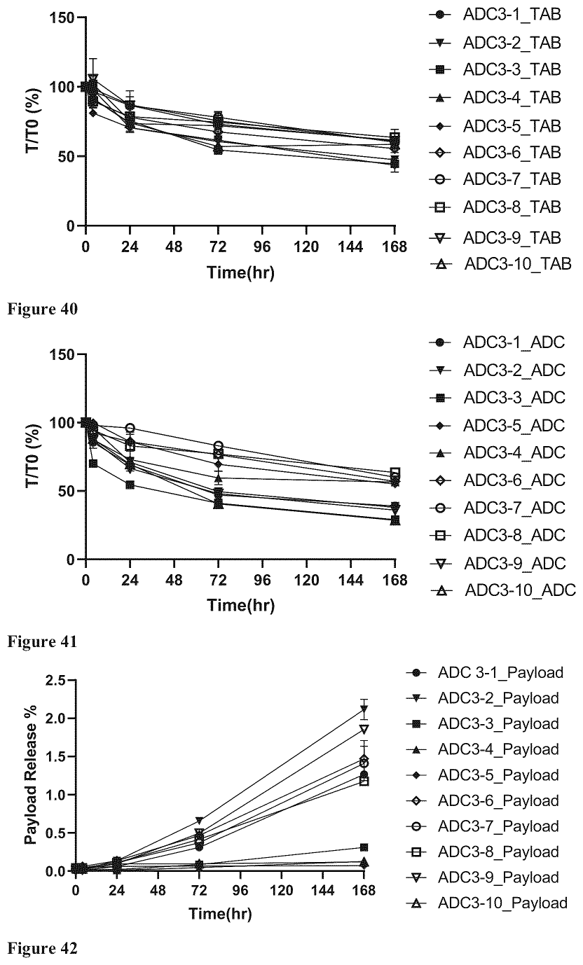

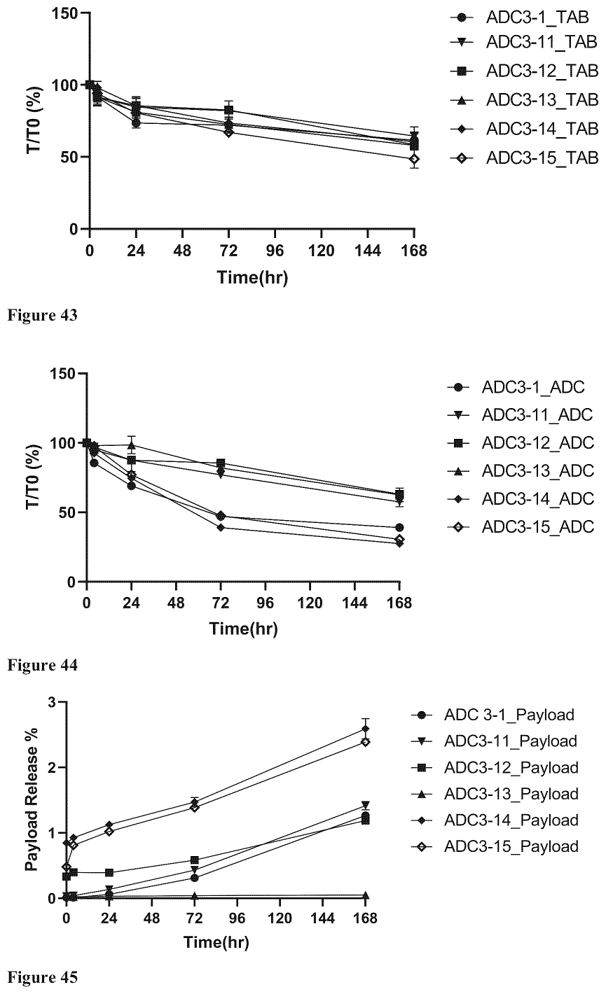

FIG. 37 illustrates ADC DAR changes of ADC2-B, ADC2-32, and ADC2-40, and ADC2-42 in rat PK.

### Compounds Cellular Killing in A375 and Calu-6 Cancer Lines

### A375 (ATCC, CRL-1619)

A375 is a cell line exhibiting epithelial morphology that was isolated from the skin of a 54-year-old, female patient with malignant melanoma, and A375 was purchased from ATCC. The base medium for A375 is DMEM, high glucose, GlutaMAX™ Supplement (Gibco, 10566024). To make the complete growth medium, ass the following components to the base medium: fetal bovine serum to a final concentration of 10% (Gibco, 10099-141C). The cell line was grown in a humidified 5% CO2 atmosphere at 37° C., and was regularly tested for the presence of mycoplasma with MycoAlert™ PLUS Mycoplasma Detection Kit (Lonza, LT07-710).

### Calu-6 (ATCC, HTB-56)

Calu-6 is a cell line exhibiting epithelial morphology that was isolated from the carcinoma and anaplastic, and Calu-6 was purchased from ATCC. The base medium for Calu-6 is Eagle's Minimum Essential Medium (ATCC, 30-2003). To make the complete growth medium, ass the following components to the base medium: fetal bovine serum to a final concentration of 10% (Gibco, 10099-141C). The cell line was grown in a humidified 5% CO2 atmosphere at 37° C., and was regularly tested for the presence of mycoplasma with MycoAlert™ PLUS Mycoplasma Detection Kit (Lonza, LT07-710).

### MDA-MB-453 (SIBS)

MDA-MB-453 was derived from an effusion of a 48 year old female patient with metastatic carcinoma of the breast, involving the nodes, brain and both pleural and pericardial cavities, and MDA-MB-453 was purchased from SIBS. The base medium for MDA-MB-453 is RPMI 1640 Medium, HEPES (Gibco, 22400105). To make the complete growth medium, ass the following components to the base medium: fetal bovine serum to a final concentration of 10% (Gibco, 10099-141C). The cell line was grown in a humidified 5% CO2 atmosphere at 37° C., and was regularly tested for the presence of mycoplasma with MycoAlert™ PLUS Mycoplasma Detection Kit (Lonza, LT07-710).

### Method: Compounds Cellular Killing

Seeding cells A375 (1E3/well) or Calu-6 (2E3/well) into 96-well plate (Greiner: 655090), 100 ul/well. Incubation at 37° C., 5% CO₂, overnight.

Adding the fresh growth-medium containing the varying concentrations of compounds, 50 ul/well.

Incubation at 37° C., 5% CO₂, 6 days.

The cell viability detected by Cell Titer-Glo (Promega, G7573). Add 70 L/well of CellTiter-Glo® Reagent in each well.

Allow the plates to incubate at room temperature for 10 minutes to stabilize luminescent signal.

Analyze with Microplate Reader.

### Result

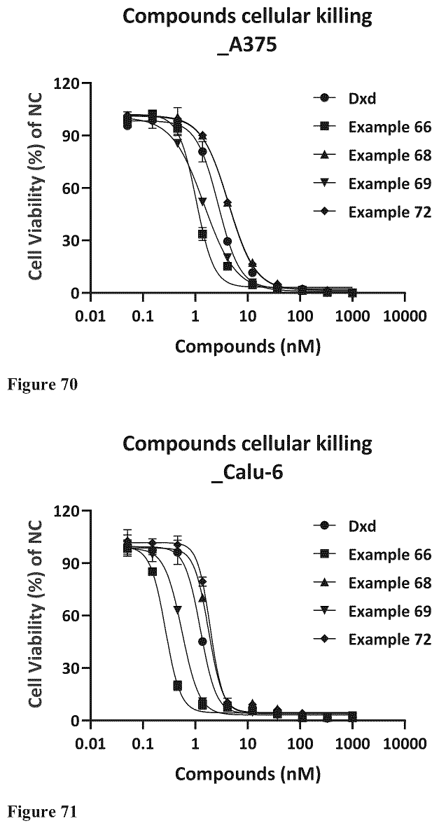

FIG. 70 and FIG. 71 illustrate that Example 66 & Example 69 showed stronger cell-killing activity than Dxd in A375 and Calu-6 cancer cell lines and that Example 68 & Example 72 were less potent than Dxd in A375 and Calu-6 cancer cell lines. Table 21 provides the corresponding data of Dxd and Examples 66, 68, 69, and 72.

TABLE 21

| | A375 | | Calu-6 | |
|---|---|---|---|---|
| Compounds | Eₘₐₓ (%) | EC₅₀ (nM) | Eₘₐₓ (%) | EC₅₀ (nM) |
| Dxd | 100.0 | 2.8 | 98.0 | 1.3 |
| Example 66 | 99.9 | 1.1 | 97.3 | 0.3 |
| Example 68 | 99.9 | 4.5 | 98.5 | 1.8 |
| Example 69 | 99.9 | 1.5 | 98.2 | 0.6 |
| Example 72 | 99.9 | 4.4 | 98.3 | 2.0 |

### ADCs Direct Killing in MDA-MB-453, A375 and Calu-6 Cancer Lines

### Method: ADCs Direct Killing

Seeding cells MDA-MB-453 or A375 (1E3/well) into 3D-96-well plate (Corning: 4520), 80 ul/well. And Calu-6 (2E3/well) into 2D-96-well plate (Greiner: 655090), 100 ul/well. Incubation at 37° C., 5% CO₂, overnight.

Adding the fresh growth-medium containing the varying concentrations of ADCs, 40 ul/well into MDA-MB-453 or A375 and 50 ul/well into Calu-6.

Incubation at 37° C., 5% CO₂, 6 days.

The MDA-MB-453 or A375 cell viability detected by 3D reagent (Promega, G9683), 100 ul/well of 3D reagent. Calu-6 cell viability detected by Cell Titer-Glo (Promega, G7573), 70 μL/well of Cell Titer-Glo® Reagent.

Allow the 3D-plates to incubate at room temperature for 30 minutes to stabilize luminescent signal, and the 2D-plates to incubate at room temperature for 10 minutes to stabilize luminescent signal.

Analyze with Microplate Reader.

### Result

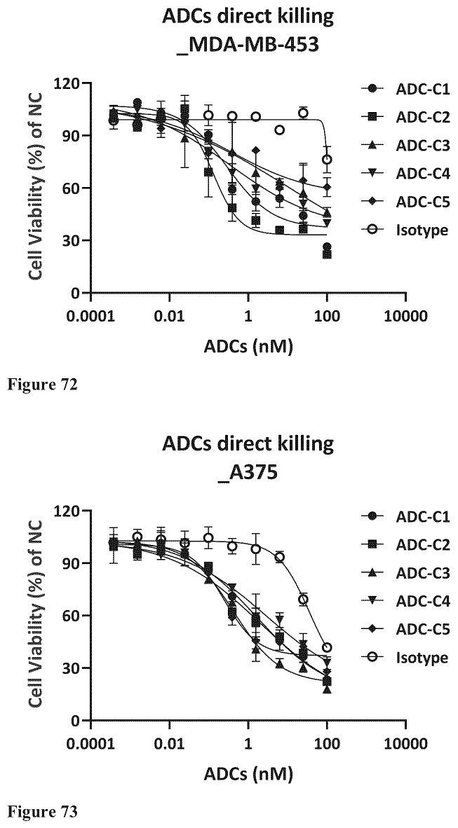

FIG. 72, FIG. 73, and FIG. 74 illustrate that ADC-C2 has stronger killing potency in MDA-MB-453 than ADC-C1, and that ADC-C3 & ADC-C5 show better cell-killing activity in A375 than ADC-C1. Table 22 provides the corresponding data of ADC-C1, ADC-C2, ADC-C3, ADC-C4, and ADC-C5.

TABLE 22

| | MDA-MB-453 | | A375 (Her3++) | |
|---|---|---|---|---|
| | (Her3+++), 3D | | 3D | |
| ADCs | Eₘₐₓ (%) | EC₅₀ (nM) | Eₘₐₓ (%) | EC₅₀ (nM) |
| ADC-C1 | 73.6 | 0.6 | 76.8 | 1.3 |
| ADC-C2 | 78.1 | 0.2 | 77.8 | 1.1 |
| ADC-C3 | 53.9 | 1.3 | 81.9 | 0.5 |
| ADC-C4 | 60.3 | 0.5 | 67.2 | 1.3 |
| ADC-C5 | 39.5 | 0.4 | 72.0 | 0.3 |

ADCs Bystander Killing MDA-MB-453 or A375 Co-Culture with Calu-6-nanoLuc

### Method: Calu-6-nanoLuc Cell Line Construction

PT67-nanoLuc cell culture, then collect the cell-culture medium (contain the virus_nano-Luc gene) and do the filtration.

Seeding the Calu-6 in the 6-well plate, 1E5 cells/well. Incubation at 37° C., 5% CO₂, overnight.

Adding the PT67-nanoLuc cell medium, and 8 ug/ml polybrene.

The infection was repeated for 3 times, every one day.

Then culture the Calu-6-nanoLuc cells, adding 1 mg/ml Geneticin for 5 days.

Collect the Calu-6-nanoLuc cells, adding Nano-Glo reagent (Promega: N1120) to test the nano-Luc transfection efficiency.

### Method: ADCs Bystander Killing

Co-culture MDA-MB-453 & Calu-6-nanoLuc (10:1), A375 & Calu-6-nanoLuc (10:1), or Calu-6-nanoLuc only into 3D-96-well plate (Corning: 4520), 80 ul/well, Incubation at 37° C., 5% CO2, overnight.

Adding the fresh growth-medium containing the varying concentrations of ADCs, 40 ul/well into cells.

Incubation at 37° C., 5% CO₂, 6 days.

Centrifuge the 3D-plates at 1500 rpm, 25° C., 5 min, then discard the supernatant.

The Calu-6-nanoLuc cell viability detected by Nano-Glo reagent (Promega: N1120), 150 ul/well.

Allow the 3D-plates to incubate at room temperature for 10 minutes to stabilize luminescent signal.

Analyze with Microplate Reader.

### Result

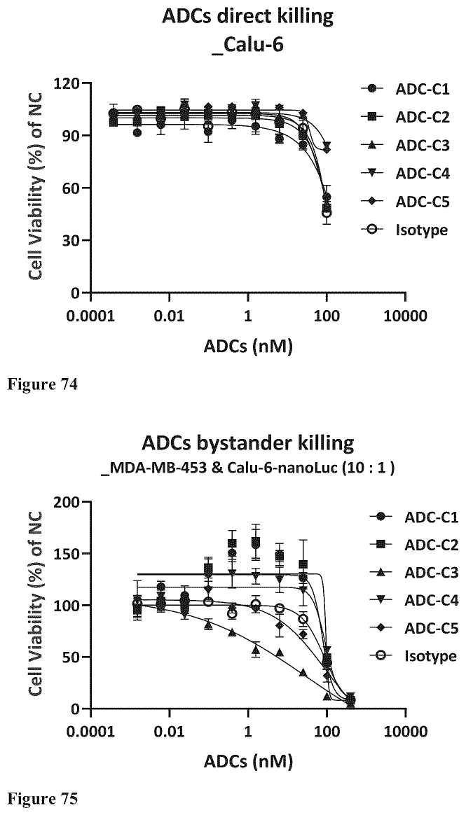

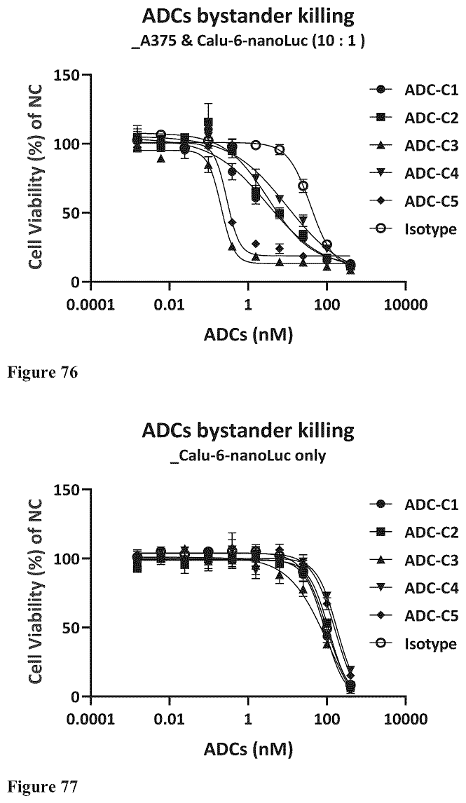

FIG. 75, FIG. 76, and FIG. 77 illustrate that ADC-C3 has stronger bystander cell-killing activity in MDA-MB-453 & Calu-6-nanoLuc system than ADC-C1, and that ADC-C3 & ADC-C5 show better bystander killing potency in A375 & Calu-6-nanoLuc system than ADC-C1. Table 23 provides the corresponding data of ADC-C1, ADC-C2, ADC-C3, ADC-C4, and ADC-C5.

TABLE 23

| | A375: | |
|---|---|---|
| | Calu-6-nanoLuc = | |
| | 10:1, 3D | |
| ADCs | Eₘₐₓ (%) | EC₅₀ (nM) |
| ADC-C1 | 86.6 | 3.1 |
| ADC-C2 | 88.6 | 4.2 |
| ADC-C3 | 91.4 | 0.2 |
| ADC-C4 | 87.6 | 8.8 |
| ADC-C5 | 89.1 | 0.3 |

### ADC Plasma Stability

Incubation of ADC with Plasma
Dilute ADC into mouse or human plasma to yield a final solution of 100 g/mL ADC in plasma
Incubate the samples at 37° C.
Aliquots (100 L) were taken at four time points (0, 4, 24, 72, 96, or 168 h)
Samples are frozen at −80° C. until analysis.
   Plasma Payload Concentrations were Carried Out Under the Following Measurement Conditions:
Instrument: LC-MS/MS (Triple Quad 5500)
Monitor: MRM
Column: Advanced Materials Technology, HALO AQ-C18 2.7 μm 90 Å, 50*2.1 mm
Column temperature: 40° C.
Mobile phase A: H2O-0.1% FA
Mobile phase B: ACN-0.11% FA
Gradient program for MMAE: 20% B-20% B (0 min-0.2 min), 20% B-80% B (0.2 min-1.5 min), 80% B-80% B (1.5 min-2.2 min), 80% B-20% B (2.20 min-2.21 min), 20% B-20% B (2.21 min-3.0 min); Gradient program for Dxd: 2% B-2% B (0 min-0.2 min), 2% B-98% B (0.2 min-1.2 min), 98% B-98% B (1.2 min-2.0 min), 98% B-2% B (2.0 min-2.01 min), 2% B-2% B (2.01 min-4.0 min);
Injected sample amount: 10 L(DXd or DXd analogues)
   Plasma ADC and Total Ab(Tab) Concentrations were Carried Out Under the Following Measurement Conditions:
Assay: Ligand binding assay (ELISA)
Capture reagent: Her3ECD
Detection reagent: anti-payload Ab for ADC and anti-human IgG polyclonal Ab for Total Ab.

### In Mice Efficacy Experiment

Female BALB/c nude mice were subcutaneously implanted with 1×10⁷ MX-1 cells per 200 μL PBS/matrigel in the right flank. After inoculation, tumor volumes were determined twice weekly in two dimensions using a caliper, and were expressed in mm³ using the formula: V=0.5(a×b²) where a and b are the long and short diameters of the tumor, respectively. When tumors reached a mean volume of approximately 230 mm³ in size, mice were randomly allocated into 4 groups with 8 animals in each group, and were intravenously treated with vehicle, ADC3-1, ADC3-3, or ADC3-4 at 10 mg/kg on day 1 and day 11, respectively. Partial regression (PR) was defined as tumor volume smaller than 50% of the starting tumor volume on the first day of dosing in three consecutive measurements and complete regression (CR) was defined as tumor volume less than 14 mm³ in three consecutive measurements. Data is presented as mean tumor volume±standard error of the mean (SEM). Tumor growth inhibition (TGI) is calculated using formula (TGI).

### In Mice PK Study

Blood samples were collected at 0, 2, 4, 8, 24, 72, and 168 h after 10 mg/kg intravenously administration of ADC-C1, ADC-C3, ADC-C4 and ADC-C5 from A375 tumor bearing mice, followed by centrifugation (4° C., 3000×g, 7 min) to separate plasma. The concentrations of ADCs and total Abs were measured by in-house developed Meso Scale Discovery (MSD) ligand binding methods. Briefly, his tagged Her3 extracellular domain fusion protein was used as a capture reagent, biotin labelled anti-DXd Ab, or goat anti-human kappa Ab were used as the detection reagents for ADCs or total Ab measurement, respectively.

### Methods

Female BALB/c nude mice were subcutaneously implanted with 3×10⁶ A-375 cells per 200 μL PBS in the right flank. After inoculation, tumor volumes were determined twice weekly in two dimensions using a caliper, and were expressed in mm³ using the formula: V=0.5(a×b²) where a and b are the long and short diameters of the tumor, respectively. When tumors reached a mean volume of approximately 290 mm³ in size, mice were randomly allocated into 4 groups with 7 animals in each group, and were intravenously treated with vehicle, ADC-C1, ADC-C3, or ADC-C5 at 10 mg/kg on day 1. Partial regression (PR) was defined as tumor volume smaller than 50% of the starting tumor volume on the first day of dosing in three consecutive measurements and complete regression (CR) was defined as tumor volume less than 14 mm³ in three consecutive measurements. Data is presented as mean tumor volume±standard error of the mean (SEM). Tumor growth inhibition (TGI) is calculated using formula (TGI).

### Results

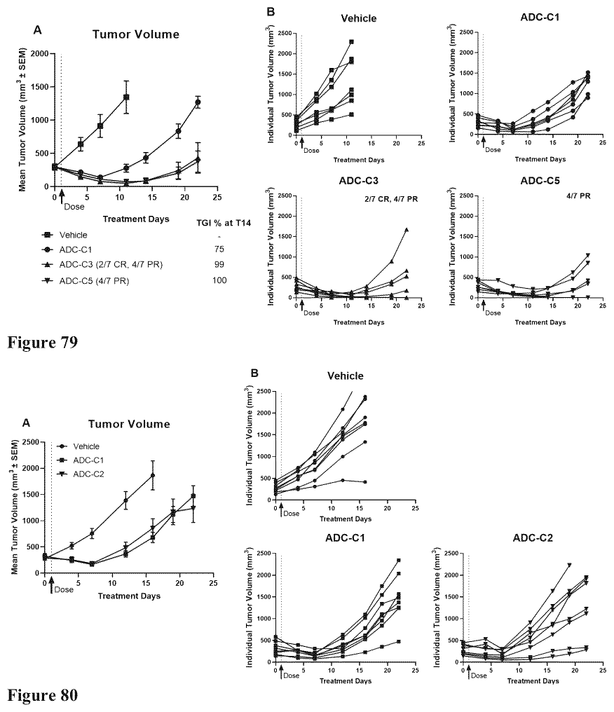

The in vivo efficacy of different ADCs were compared in A-375 melanoma xenografts grown subcutaneously in BALB/c nude mice. Results are shown in FIG. 79. Treatment with ADC-C1, ADC-C3, or ADC-C5 at 10 mg/kg resulted in 75%, 99%, or 100% tumor growth inhibition (TGI) on day 14, respectively (FIG. 79A). For individual animal tumor growth inhibition, ADC-C1 didn't induce tumor PR or CR, while ADC-C3 induced 2/7 CR and 4/7 PR, ADC-C5 induced 4/7 PR (FIG. 79B). Meanwhile all three ADCs were well tolerated without any sign of toxicity or significant body weight decrease. Taken together, ADC-C3 and ADC-C5 demonstrated better PR and CR ratio than ADC-C1.

### Methods

Female BALB/c nude mice were subcutaneously implanted with 3×10⁶ A-375 cells per 200 μL PBS in the right flank. When tumors reached a mean volume of approximately 280 mm³ in size, mice were randomly allocated into 3 groups with 8 animals in each group, and were intravenously treated with vehicle, ADC-C1, or ADC-C2 at 10 mg/kg on day 1, respectively.

### Result

The in vivo efficacy of different ADCs were compared in A-375 melanoma xenografts grown subcutaneously in BALB/c nude mice. Results are shown in FIG. 80. Treatment with ADC-C2 resulted in comparable anti-tumor efficacy with ADC-C1 at 10 mg/kg both mean tumor volume and individual tumor volume. The data was provided in FIG. 80 (FIGS. 80A and 80B). Meanwhile both ADCs were well tolerated without any sign of toxicity or significant body weight decrease.

TABLE 24
ADC Examples

| ADC | |
|---|---|
| num- | |
| ber | ADC structure |
| ADC- C6 | 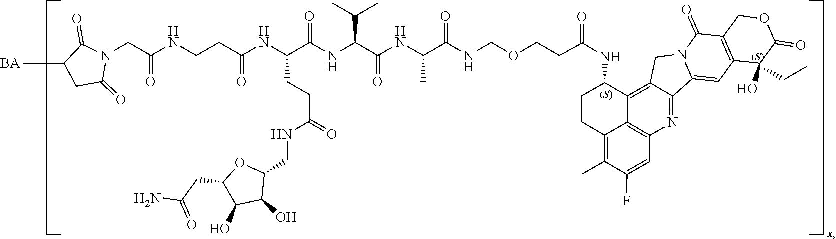 |
| ADC- C7 | 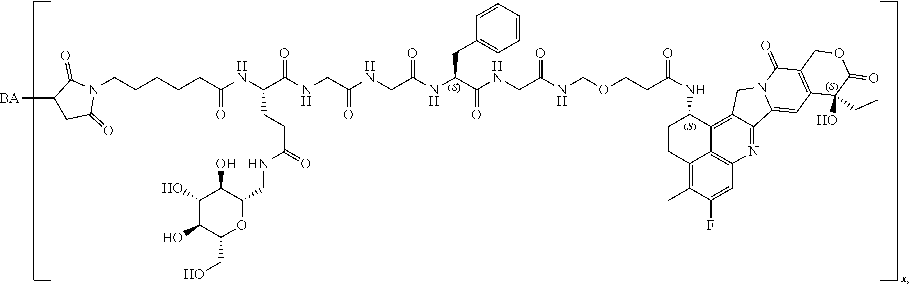 |
| ADC- C3 | 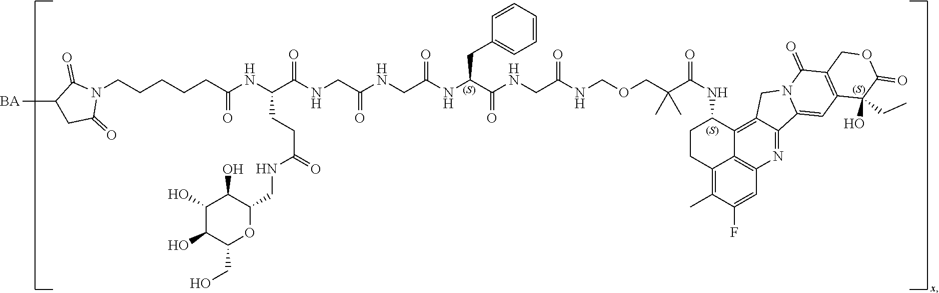 |
| | or |
| ADC- C4 | 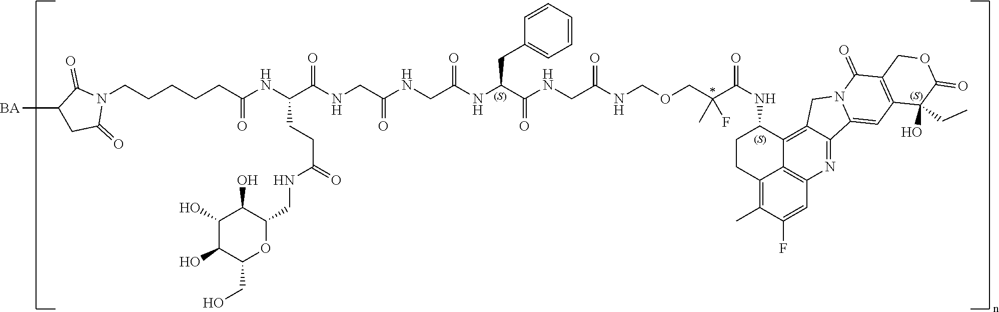 |
| | Isomer 1 |
| ADC- C5 | 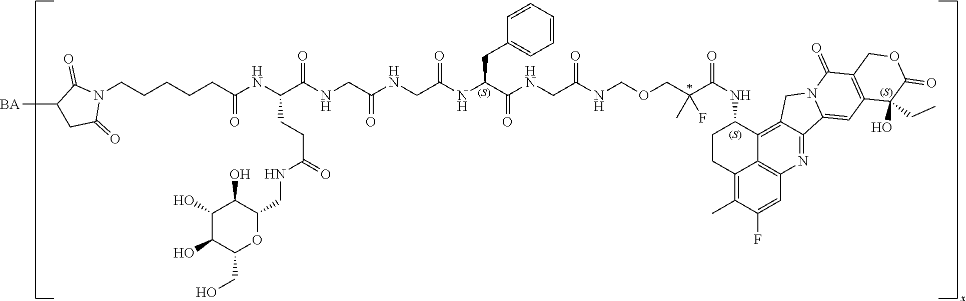 |
| | Isomer 2 |

TABLE 25
Linker-Payload Examples

| Linker- | |
|---|---|
| Payload | |
| number | Linker-Payload Structure |
| Com- pound 74 | 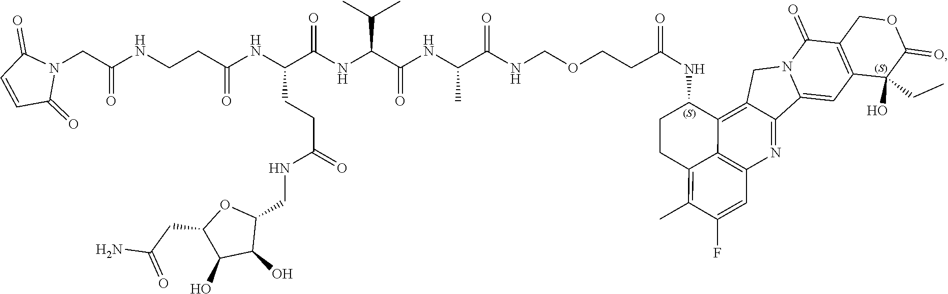 |
| Com- pound 73 | 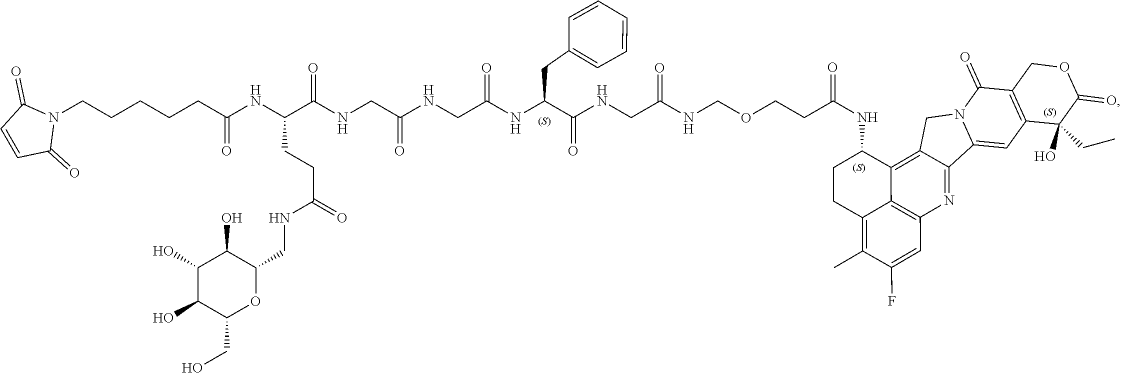 |
| Com- pound 67 | 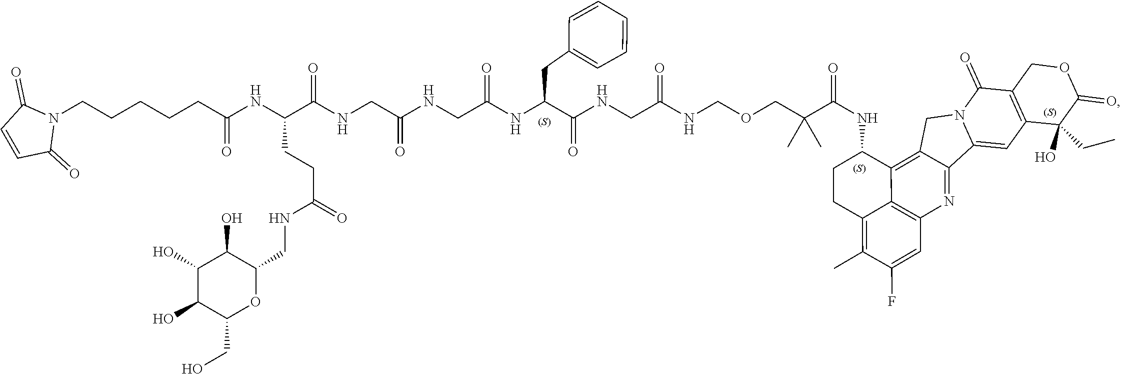 |
| | or |
| Isomer 1 of Compound 70 & 71 | 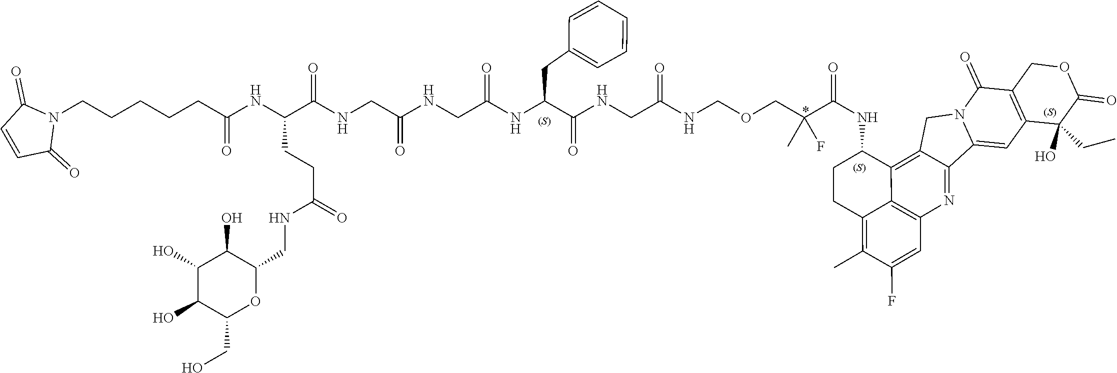 |
| | Isomer 1 |
| Isomer 2 of Compound 70 & 71 | 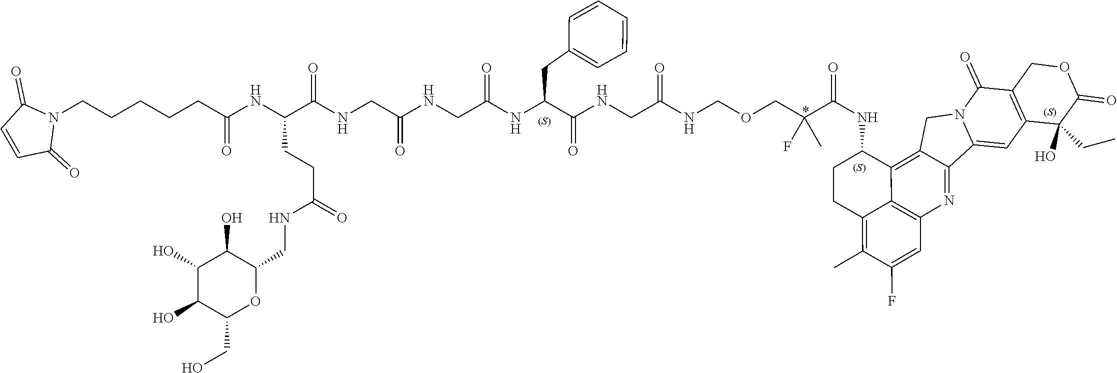 |
| | Isomer 2 |

TABLE 26
Toxin Examples

| Compound No. | Structures |
|---|---|
| Compound 72 | 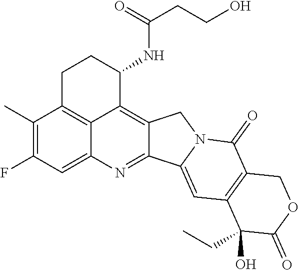 [SMILES: CC[C@@]1(O)C(=O)OCc2c1cc1n(c2=O)Cc2c-1nc1cc(F)c(C)c3c1c2[C@@H](NC(=O)CCO)CC3] |
| Compound 66 | 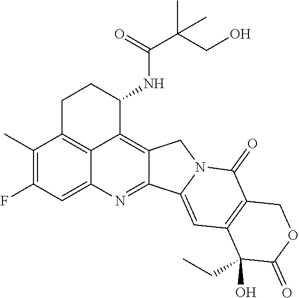 [SMILES: CC[C@@]1(O)C(=O)OCc2c1cc1n(c2=O)Cc2c-1nc1cc(F)c(C)c3c1c2[C@@H](NC(=O)C(C)(C)CO)CC3] |
| Isomer 1 of Compound 68 & 69 | 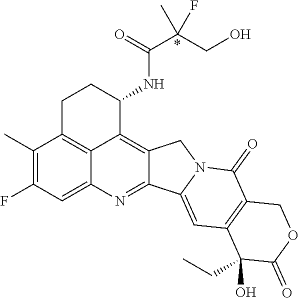 [SMILES: CC[C@@]1(O)C(=O)OCc2c1cc1n(c2=O)Cc2c-1nc1cc(F)c(C)c3c1c2[C@@H](NC(=O)C(C)(F)CO)CC3] |
| | Isomer 1 |
| Isomer 2 of Compound 68 & 69 | 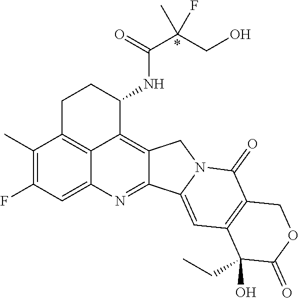 [SMILES: CC[C@@]1(O)C(=O)OCc2c1cc1n(c2=O)Cc2c-1nc1cc(F)c(C)c3c1c2[C@@H](NC(=O)C(C)(F)CO)CC3] |
| | Isomer 2 |

TABLE 27

| | A375, 2D | | Calu-6, 2D | | | |
|---|---|---|---|---|---|---|
| Free-payload | (Her3 ++) | | (HER3 neg.) | | PAMPA | |
| ID/Structure | Eₘₐₓ (%) | EC₅₀ (nM) | Eₘₐₓ (%) | EC₅₀ (nM) | -Log Pe | Recovery % |
| 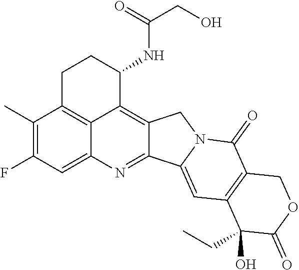 [SMILES: CC[C@@]1(O)C(=O)OCc2c1cc1n(c2=O)Cc2c-1nc1cc(F)c(C)c3c1c2[C@@H](NC(=O)CO)CC3] | 100.0 100.0 | 2.8 2.7 | 98.0 98.8 | 1.3 1.3 | 5.89 | 95.15 |
| DXd | | | | | | |
| 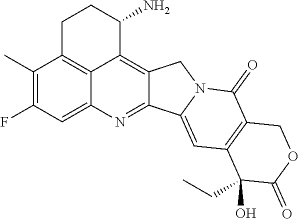 [SMILES: CC[C@@]1(O)C(=O)OCc2c1cc1n(c2=O)Cc2c-1nc1cc(F)c(C)c3c1c2[C@@H](N)CC3] | 99.9 | 0.7 | 96.2 | 0.3 | 5.75 | 56.05 |
| Exatecan | | | | | | |
| 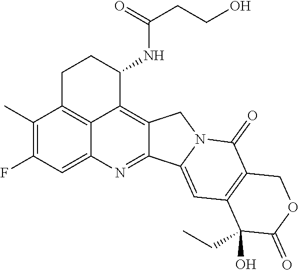 [SMILES: CC[C@@]1(O)C(=O)OCc2c1cc1n(c2=O)Cc2c-1nc1cc(F)c(C)c3c1c2[C@@H](NC(=O)CCO)CC3] | 99.9 | 4.4 | 98.3 | 2.0 | 6.02 | 48.13 |
| Compound 72 | | | | | | |
| 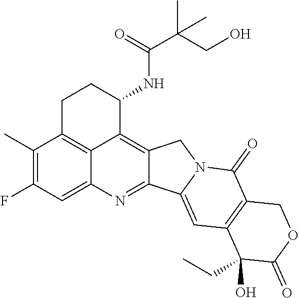 [SMILES: CC[C@@]1(O)C(=O)OCc2c1cc1n(c2=O)Cc2c-1nc1cc(F)c(C)c3c1c2[C@@H](NC(=O)C(C)(C)CO)CC3] | 99.9 | 1.1 | 97.3 | 0.3 | 5.89 | 79.71 |
| Compound 66 | | | | | | |

TABLE 28

| | | | MDA-MB-453, | | A375, 2D (Her3 | | Calu-6, 2D | |
|---|---|---|---|---|---|---|---|---|
| | HCC1569, 2D | | 2D (HER3 +++) | | ++) | | (HER3 neg.) | |
| Payload | (HER3 +++) | | Eₘₐₓ | EC₅₀ | | EC₅₀ | | EC₅₀ |
| ID/Structure | Eₘₐₓ (%) | EC₅₀ (nM) | (%) | (nM) | Eₘₐₓ (%) | (nM) | Eₘₐₓ (%) | (nM) |
| 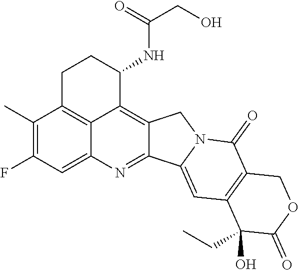 [SMILES: CC[C@@]1(O)C(=O)OCc2c1cc1n(c2=O)Cc2c-1nc1cc(F)c(C)c3c1c2[C@@H](NC(=O)CO)CC3] | 93.4 | 2.9 | 98.7 | 1.9 | 100.0 | 3.2 | 98.6 | 1.2 |
| DXd | | | | | | | | |
| 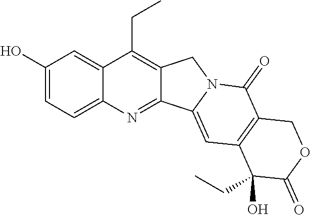 [SMILES: CCc1c2c(nc3ccc(O)cc13)-c1cc3c(c(=O)n1C2)COC(=O)[C@]3(O)CC] | 95.0 | 5.0 | 98.6 | 2.4 | 99.9 | 3.4 | 99.5 | 2.9 |
| SN-38 | | | | | | | | |
| 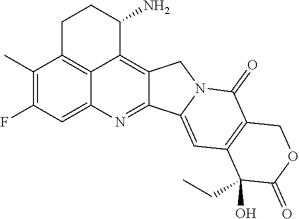 [SMILES: CC[C@@]1(O)C(=O)OCc2c1cc1n(c2=O)Cc2c-1nc1cc(F)c(C)c3c1c2[C@@H](N)CC3] | 94.5 | 0.5 | 99.4 | 0.5 | 99.9 | 0.9 | 96.4 | 0.2 |
| Exatecan | | | | | | | | |
| 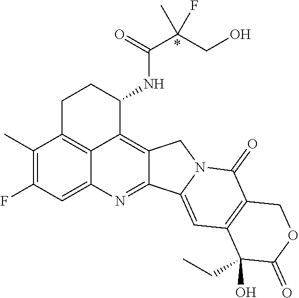 [SMILES: CC[C@@]1(O)C(=O)OCc2c1cc1n(c2=O)Cc2c-1nc1cc(F)c(C)c3c1c2[C@@H](NC(=O)C(C)(F)CO)CC3] | 92.0 | 3.2 | 97.9 | 2.8 | 99.9 | 4.5 | 98.5 | 1.8 |
| Isomer 1 of | | | | | | | | |
| Compounds 68 & | | | | | | | | |
| 69 | | | | | | | | |
| Isomer 1 | | | | | | | | |
| 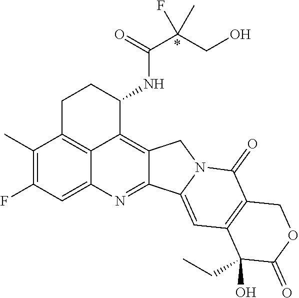 [SMILES: CC[C@@]1(O)C(=O)OCc2c1cc1n(c2=O)Cc2c-1nc1cc(F)c(C)c3c1c2[C@@H](NC(=O)C(C)(F)CO)CC3] | 91.6 | 0.7 | 98.6 | 1.5 | 99.9 | 1.5 | 98.2 | 0.6 |
| Isomer 2 of | | | | | | | | |
| Compounds 68 & | | | | | | | | |
| 69 | | | | | | | | |
| Isomer 2 | | | | | | | | |

TABLE 29

| | | | | | | | | Bystander | | Bystander | |
|---|---|---|---|---|---|---|---|---|---|---|---|
| | | | | | | | | Killing | | Killing | |
| | | | | | | | | MDA-MB- | | A375: | |
| | A375, 6d, | | Clau6, 6d, | | | | | 453:Calu- | | Calu-6- | |
| | 2D | | 2D | | | A375, 6d, 3D | | 6-nanoLuc = | | nanoLuc = | |
| | (Her3++) | | (Her3 neg) | | | (Her3++) | | 10:1, 3D | | 10:1, 3D | |
| Payload ID/ | Eₘₐₓ | EC₅₀ | Eₘₐₓ | EC₅₀ | Her3 | Eₘₐₓ | EC₅₀ | Eₘₐₓ | EC₅₀ | Eₘₐₓ | EC₅₀ |
| Structure | (%) | (nM) | (%) | (nM) | ADC | (%) | (nM) | (%) | (nM) | (%) | (nM) |
| DXd | 100.0 | 2.7 | 98.8 | 1.3 | ADC-C1 | 78.6 | 0.241 | Not | Not | 85.9 | 0.4 |
| | | | | | | 76.8 | 1.3 | observed | observed | 86.6 | 3.1 |
| | | | | | ADC-C2 | 77.8 | 1.1 | Not | Not | 88.6 | 4.2 |
| | | | | | | | | observed | observed | | |
| Compound 72 | 99.9 | 4.4 | 98.3 | 2.0 | | | | | | | |
| Compound 66 | 99.9 | 1.1 | 97.3 | 0.3 | ADC-C3 | 81.9 | 0.5 | 94.8 | 3.8 | 91.4 | 0.2 |
| Isomer 1 of | 99.9 | 4.5 | 98.5 | 1.8 | ADC-C4 | 67.2 | 1.3 | Not | Not | 87.6 | 8.8 |
| Compounds | | | | | | | | observed | observed | | |
| 68 & 69 | | | | | | | | | | | |
| Isomer 2 of | 99.9 | 1.5 | 98.2 | 0.6 | ADC-C5 | 72.0 | 0.3 | Not | Not | | |
| Compounds | | | | | | | | observed | observed | 89.1 | 0.3 |
| 68 & 69 | | | | | | | | | | | |

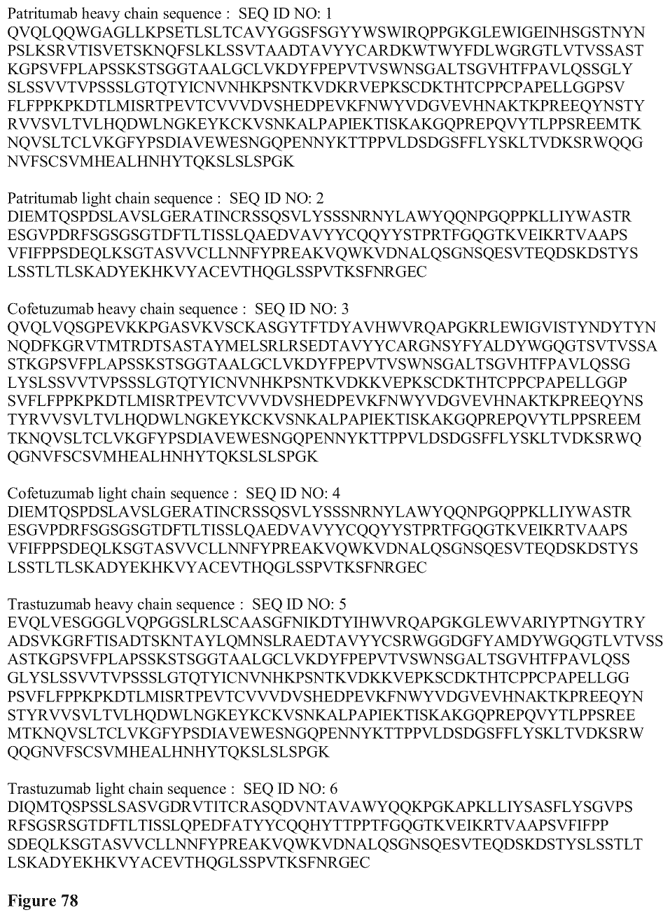

The antibodies used herein were prepared according to conventional methods, for example, vector construction, eukaryotic cell transfection such as HTEK293 cell (Life Technologies Cat. No. 11625019) transfection, purification and expression. The sequences of the antibodies used herein can be found in FIG. 78.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

It is to be understood that, if any prior art publication is referred to herein; such reference does not constitute an admission that the publication forms a part of the common general knowledge in the art in any country.

The disclosures of all publications, patents, patent applications and published patent applications referred to herein by an identifying citation are hereby incorporated herein by reference in their entirety.

1. A compound, wherein the compound is a compound of Formula (I): 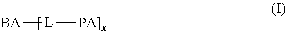
or a pharmaceutically acceptable salt tautomer, solvate, stereoisomer, enantiomer, isotopologue, or prodrug thereof,
wherein BA is a binding agent selected from a humanized, chimeric, or human antibody or an antigen binding antibody fragment of an antibody; L is a covalent linker; PA is a payload residue; and subscript x is from 1 to 30.

2-10. (canceled)

11. A compound, wherein the compound is a compound of Formula (IIa), (IIb), or (IIc): 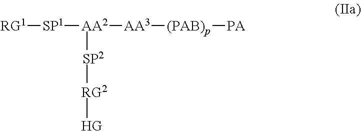
or a pharmaceutically acceptable salt tautomer, solvate, stereoisomer, enantiomer, isotopologue, or prodrug thereof, wherein:
RG¹ is a reactive group residue; RG² is an optional reactive group residue; SP¹ and SP² are independently, in each instance, an optional spacer group residue; HG is a hydrophilic residue; PAB is an optional self-immolative unit; subscript p is 0 or 1;
AA² comprises formula (W): 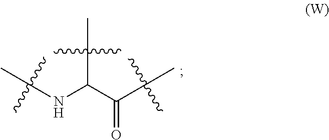 [SMILES: *NC(*)C(*)=O]
and
AA³ is a dipeptide residue of -valine-alanine-, -valine-citrulline-, or 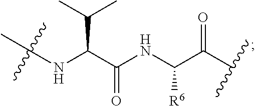
wherein R⁶ is —CH₃, or —(CH₂)₃—NHC(═O)NH₂; or
AA³ is a tetrapeptide residue of -glycine-glycine-phenylalanine-glycine- or 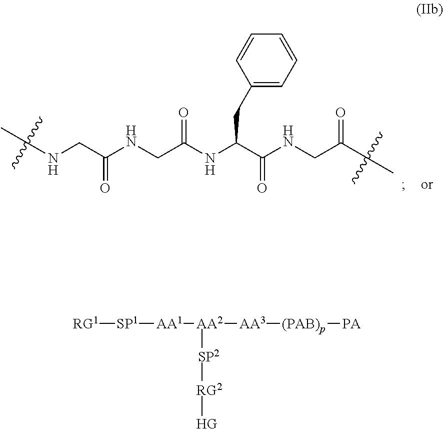
or a pharmaceutically acceptable salt tautomer, solvate, stereoisomer, enantiomer, isotopologue, or prodrug thereof, wherein:
RG¹ is a reactive group residue; RG² is an optional reactive group residue; SP¹ and SP² are independently, in each instance, an optional spacer group residue; HG is a hydrophilic residue; PAB is an optional self-immolative unit; subscript p is 0 or 1;
AA² comprises formula (W): 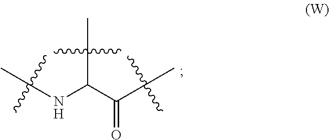 [SMILES: *NC(*)C(*)=O]
and
AA¹ is a dipeptide residue of -valine-alanine-, -valine-citrulline-, or 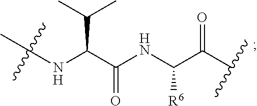
wherein R⁶ is —CH₃, or —(CH₂)₃—NHC(═O)NH₂; or
AA¹ is a tetrapeptide residue of -glycine-glycine-phenylalanine-glycine- or 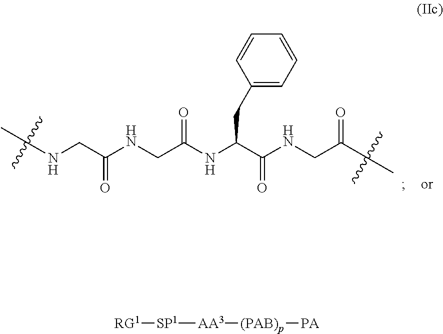
or a pharmaceutically acceptable salt tautomer, solvate, stereoisomer, enantiomer, isotopologue, or prodrug thereof, wherein:
RG¹ is a reactive group residue;
SP¹ is an optional spacer group residue;
PAB is an optional self-immolative unit;
subscript p is 0 or 1;
PA is a payload residue; and
AA³ is a dipeptide residue of -valine-alanine-, -valine-citrulline-, or 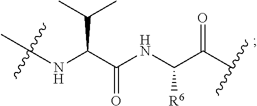
wherein R⁶ is —CH₃, or —(CH₂)₃—NHC(═O)NH₂; or
AA³ is a tetrapeptide residue of -glycine-glycine-phenylalanine-glycine- or

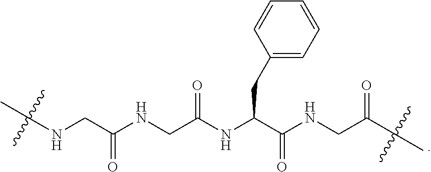 [SMILES: *NCC(=O)NCC(=O)N[C@@H](Cc1ccccc1)C(=O)NCC(*)=O]

12. The compound of claim 0, wherein the compound is a compound of Formula (IIa): 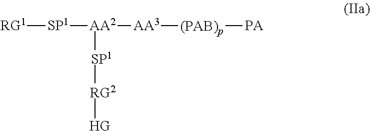
or a pharmaceutically acceptable salt tautomer, solvate, stereoisomer, enantiomer, isotopologue, or prodrug thereof.

13. The compound of claim 0, wherein the compound is a compound of Formula (IIb): 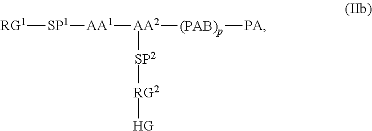
or a pharmaceutically acceptable salt tautomer, solvate, stereoisomer, enantiomer, isotopologue, or prodrug thereof.

14. The compound of claim 0, wherein the compound is a compound of Formula (IIc): 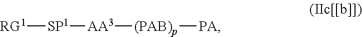
or a pharmaceutically acceptable salt tautomer, solvate, stereoisomer, enantiomer, isotopologue, or prodrug thereof.

15. The compound of claim 11, wherein RG¹ is

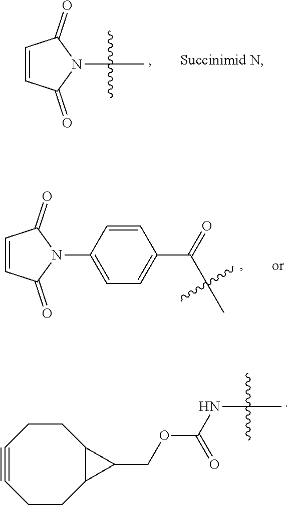 [SMILES: *N1C(=O)C=CC1=O ; *C(=O)c1ccc(N2C(=O)C=CC2=O)cc1 ; *NC(=O)OCC1C2CCC#CCCC21]

16. The compound claim 11, wherein RG¹ is 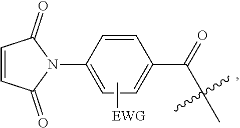
wherein EWG is an electron withdrawing group selected from the group consisting of —CN, halogen, —CF₃, —C(═O)OR¹, and —C(═O)R¹, and R¹ is substituted or unsubstituted alky, substituted or unsubstituted cycloalkyl, substituted or unsubstituted aryl, substituted or unsubstituted heterocycloalkyl, or substituted or unsubstituted heteroaryl.

17. The compound of claim 11, wherein RG¹ is

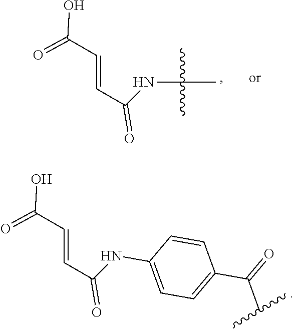 [SMILES: *NC(=O)/C=C/C(=O)O ; *C(=O)c1ccc(NC(=O)/C=C/C(=O)O)cc1]

18. The compound of claim 11, wherein RG¹ is 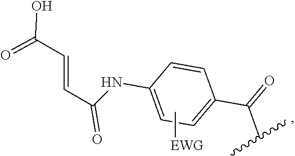
wherein EWG is an electron withdrawing group selected from —CN, halogen, —CF₃, —C(═O)OR¹, and —C(═O)R¹, and R¹ is substituted or unsubstituted alky, substituted or unsubstituted cycloalkyl, substituted or unsubstituted aryl, substituted or unsubstituted heterocycloalkyl, or substituted or unsubstituted heteroaryl.

19. The compound of claim 11, wherein SP¹ is —(CH₂)ₙ₁—C(═O)—, —(CH₂CH₂O)ₙ₂—CH₂CH₂—C(═O)—, —CH[—(CH₂)ₙ₃—COOH]—C(═O)—, —CH₂—C(═O)—NH—(CH₂)ₙ₄—C(═O)—, —CH₂—C(═O)—NH—(CH₂)ₙ₃—C(═O)—NH—(CH₂)ₙ₄—C(═O)—, or —C(═O)—(CH₂)ₙ₅—C(═O)—, wherein each of n1, n2, n3, n4, and n5 independently represents an integer of 1 to 8.

20. The compound of claim 11, wherein SP² is —(CH₂)ₙ₆—; and n6 represents an integer of 1 to 8.

21. The compound of claim 11, wherein RG² is a bond, —C(═O)—NH—, or —NHC(═O)—.

22. The compound of claim 11, wherein HG is 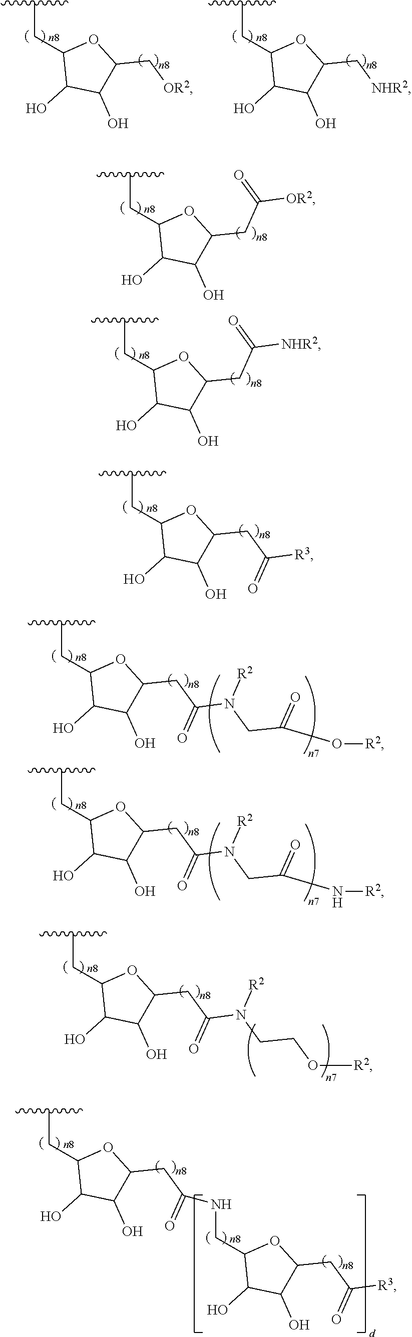 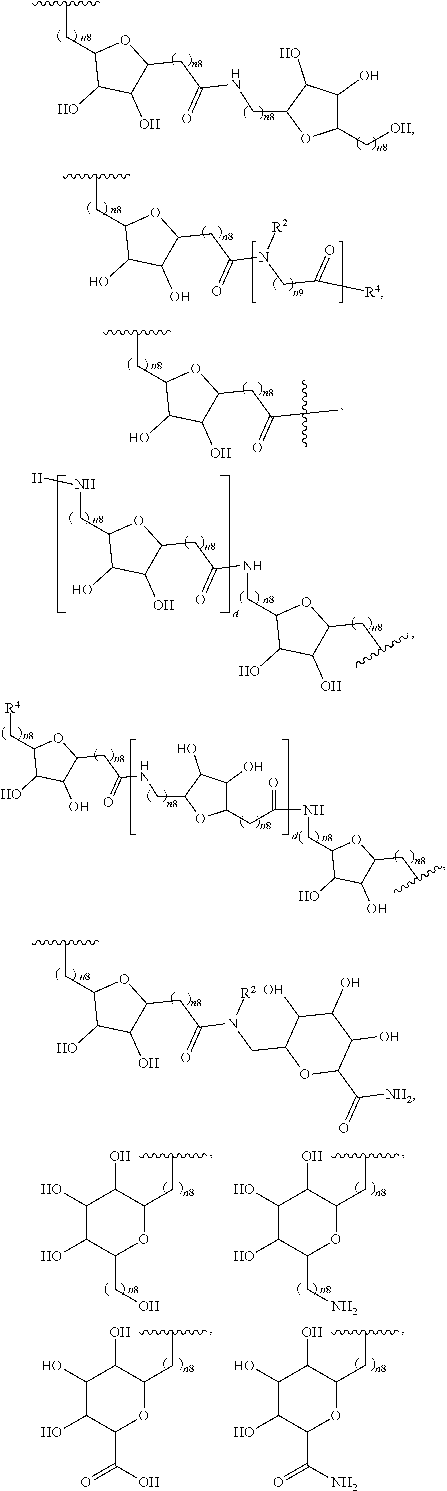 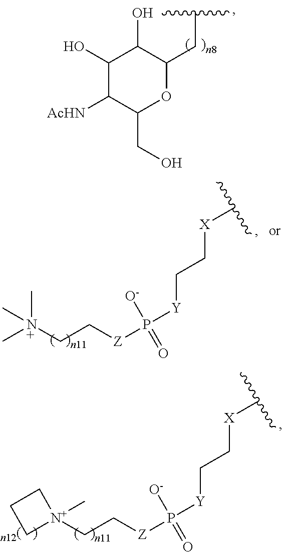
wherein each n7 is independently 1-15; each n8 is independently 0 or 1; each n9 is independently 1 or 2; each n10 is independently an integer of 4 to 16; each n11 is independently an integer of 0 to 5; n12 is an integer of 0 to 3; d is 0-3; R² is H or Me; R³ is —OH, —NH₂, —NHCH₂—CH₂—(PEG)ₓ-OH, or —NHCH₂—CH₂—(PEG)ₓ-OMe; R^{#} is OH or NH₂; and each of X, Y, and Z is independently —CH₂—, —NH—, —S— or —O—.

23. The compound of claim 11, wherein HG is —NHSO₂NH₂, —SO₃H, —SO₂NH₂, or —PO₃H₂, and RG² is a bond.

24. The compound of claim 14, wherein PAB represents —NH—CH₂—O—, formula (Y1): 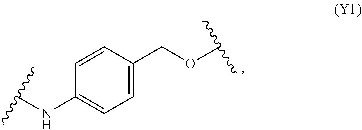 [SMILES: *Nc1ccc(CO*)cc1]
or formula (Y2): 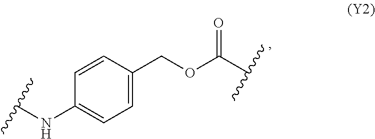 [SMILES: *Nc1ccc(COC(*)=O)cc1]
wherein the 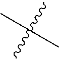
indicates the bond through which the PAB is bonded to the adjacent groups in the formula.

25. The compound of claim 11, wherein each PA independently represents formula (D1): 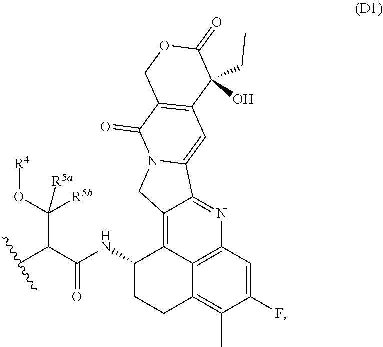
wherein each of R⁴, R^{5a}, and R^{5b} is independently hydrogen, sugar residue, substituted or unsubstituted inorganic or organic acid residue, substituted or unsubstituted C₁₋₈ alkyl, substituted or unsubstituted cycloalkyl, substituted or unsubstituted non-aromatic heterocyclyl, substituted or unsubstituted cycloalkylalkyl, or substituted or unsubstituted heterocyclylalkyl;
or R^{5a} and R^{5b} together with the atoms to which they are attached, form a substituted or unsubstituted cycloalkyl, substituted or unsubstituted non-aromatic heterocyclyl.

26. The compound of claim 11, wherein each PA independently represents formula (D2): 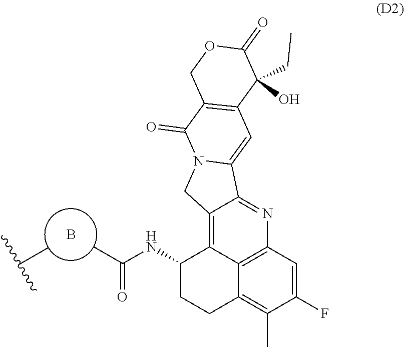
wherein ring B is a substituted or unsubstituted cycloalkyl, substituted or unsubstituted aryl, substituted or unsubstituted heterocyclyl, or substituted or unsubstituted heteroaryl.

27. The compound of claim 11, wherein each PA independently represents formula (E1): 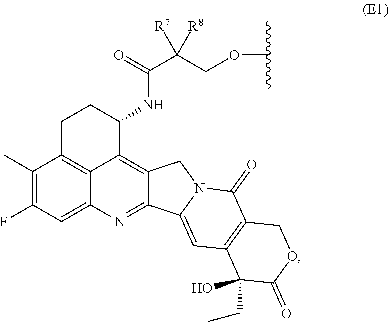
wherein
each of R⁷ and R⁸ is, independently, hydrogen, halogen, or alkyl.

28. (canceled)

29. A pharmaceutical composition comprising a compound of claim 1, or a pharmaceutically acceptable salt, tautomer, solvate, stereoisomer, enantiomer, isotopologue, or prodrug thereof, and a pharmaceutically acceptable excipient.

30. A method of treating a proliferative disease, a metabolic disease, inflammation, or a neurodegenerative disease in a subject comprising administering to the subject an effective treatment amount of a compound of claim 1, or a pharmaceutically acceptable salt, tautomer, solvate, stereoisomer, enantiomer, isotopologue, or prodrug thereof.

31. The compound of claim 11, wherein the compound is selected from: 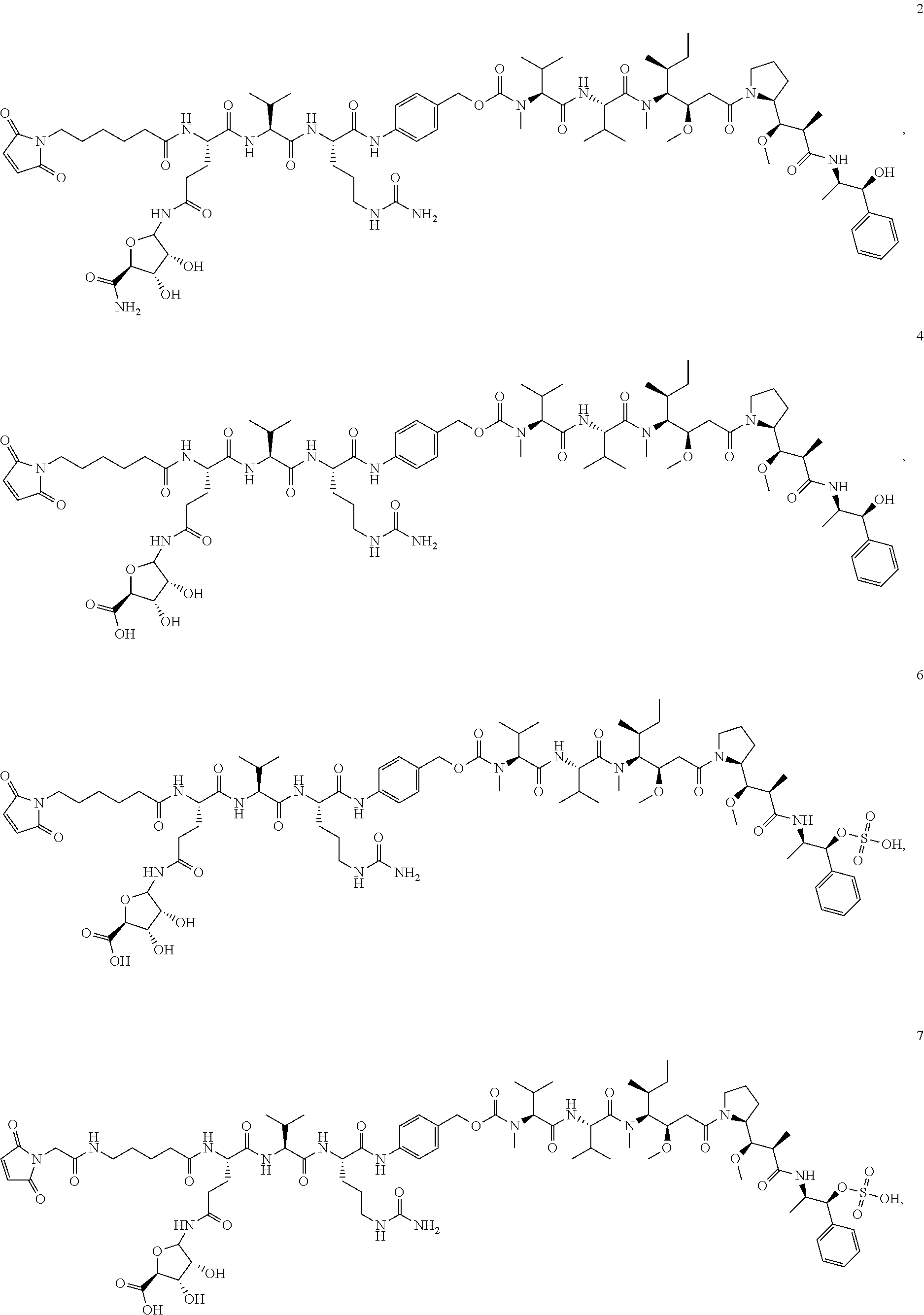 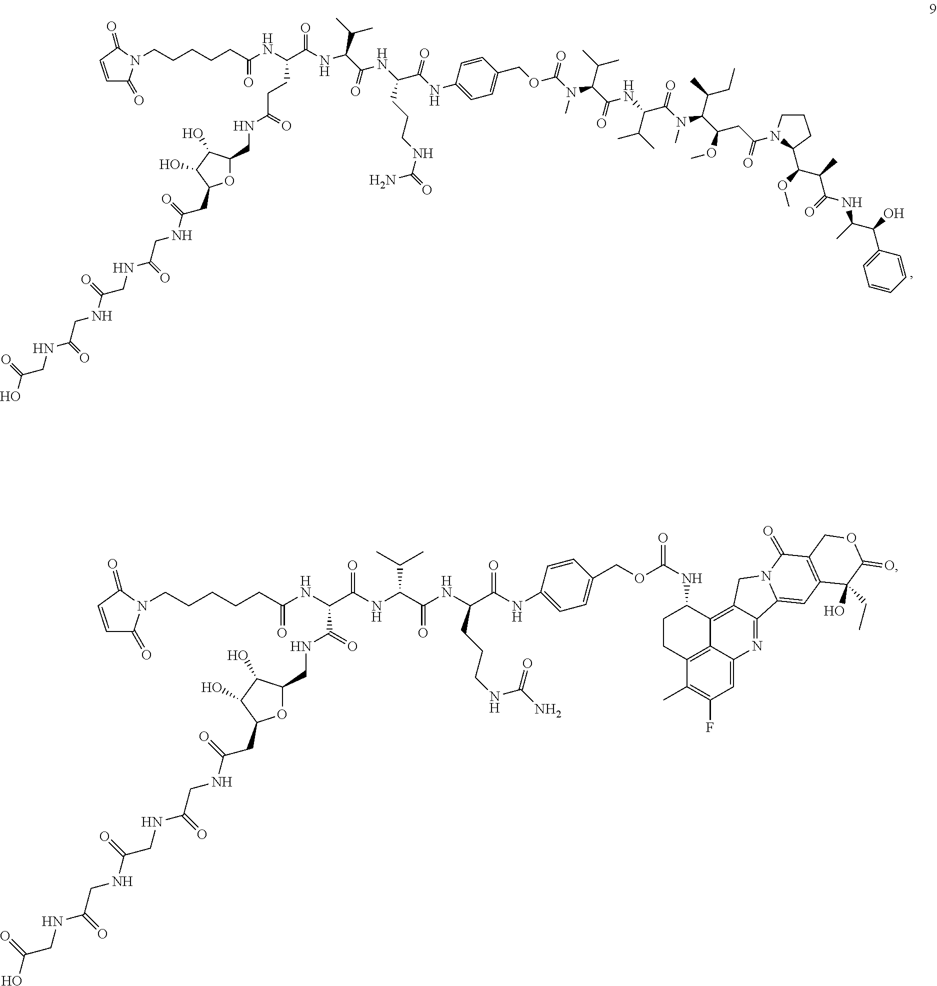 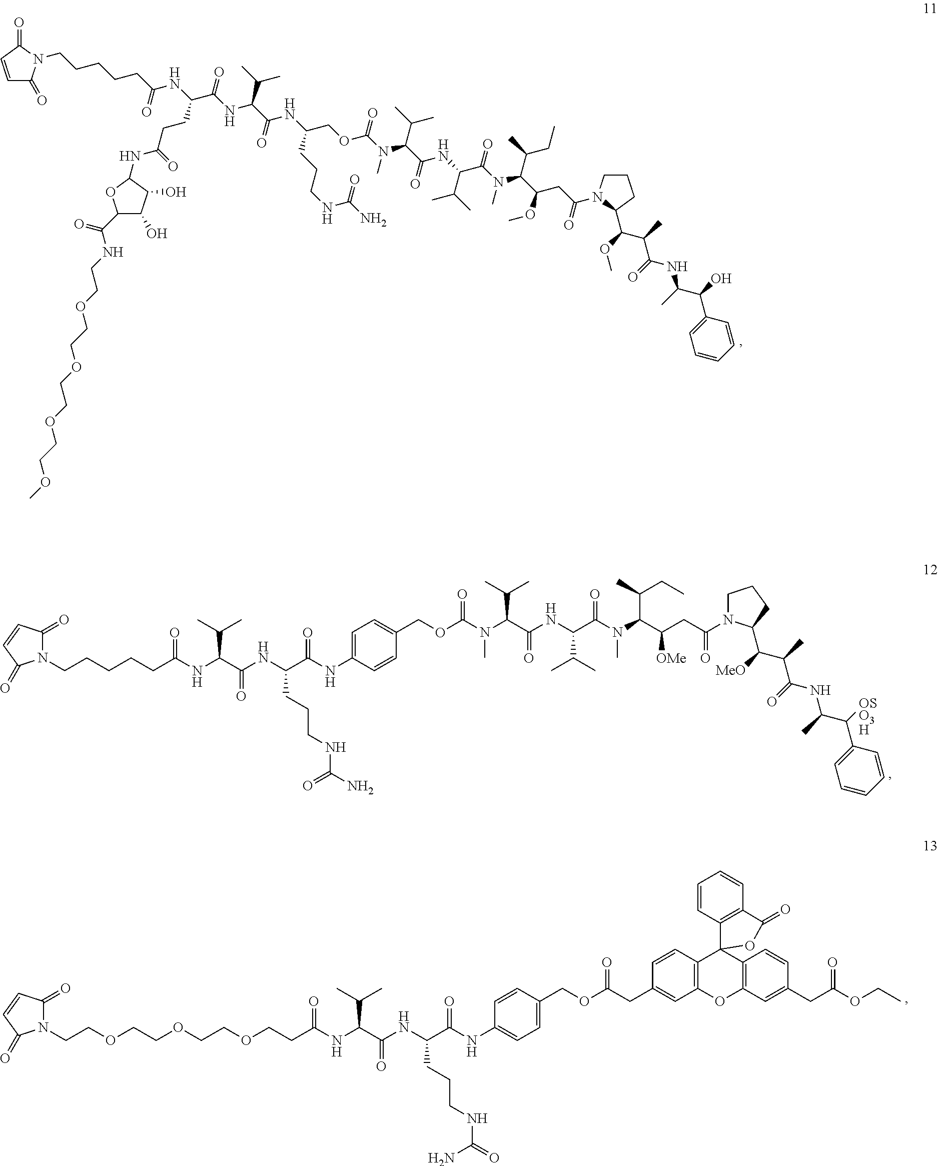 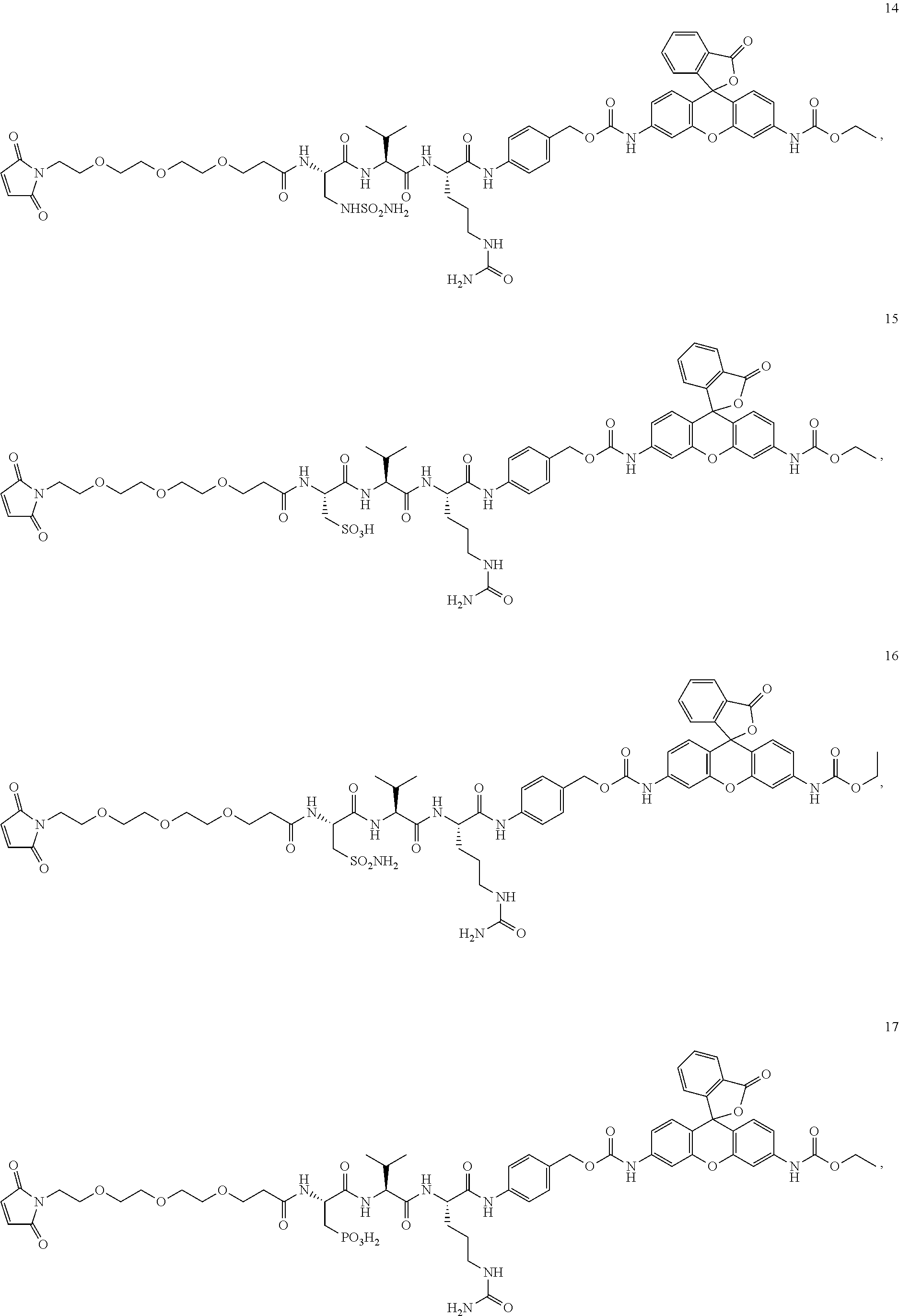 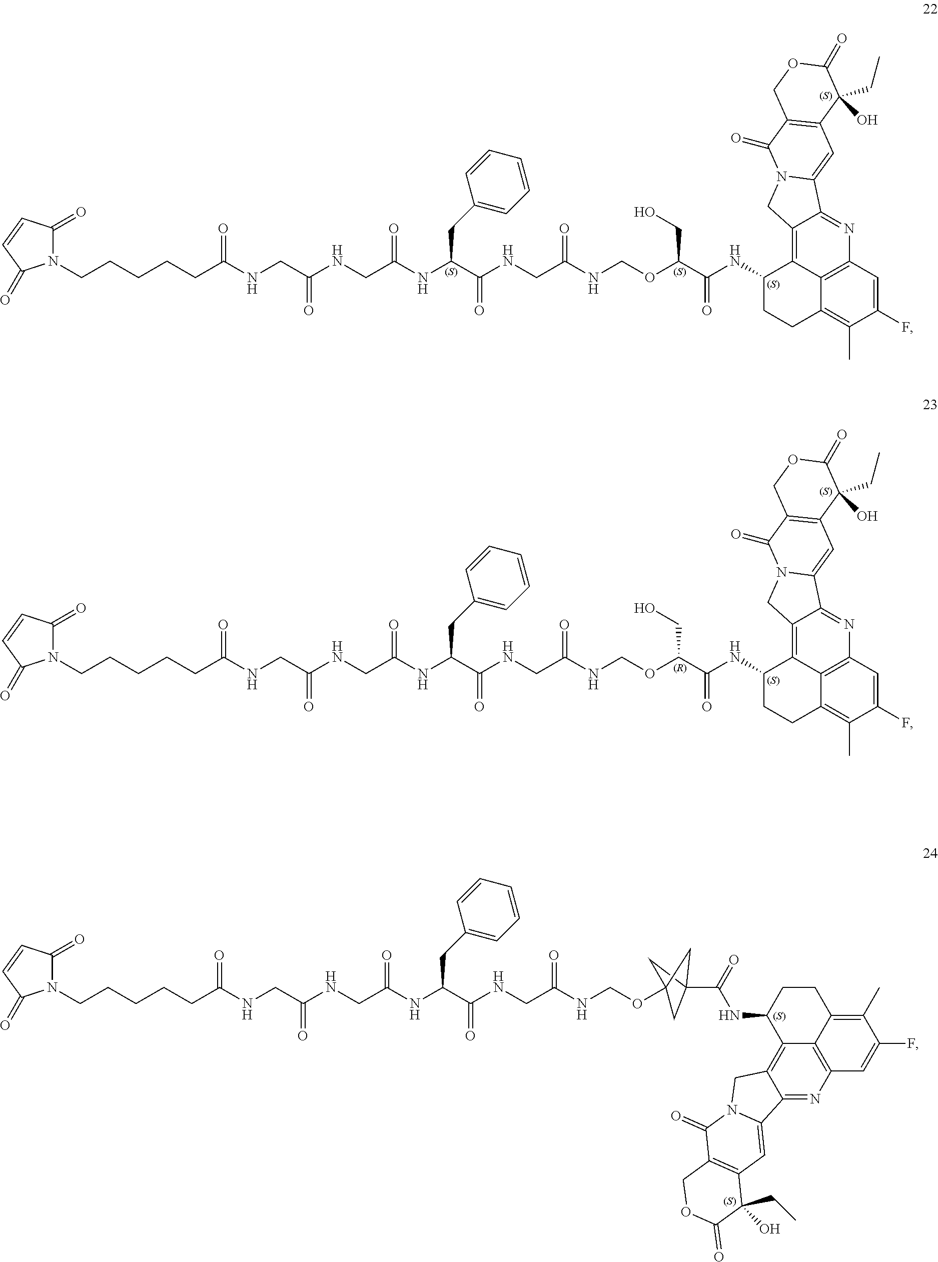 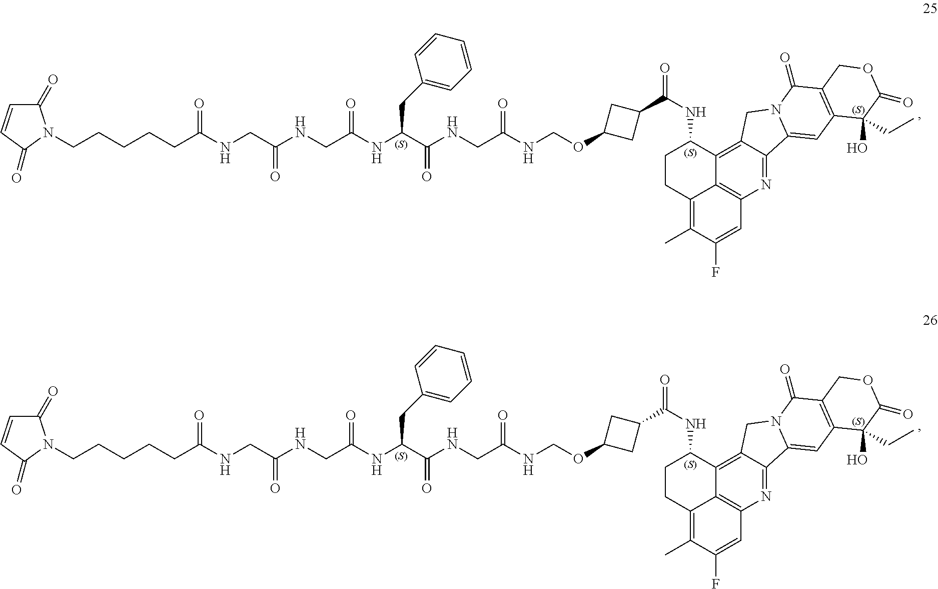 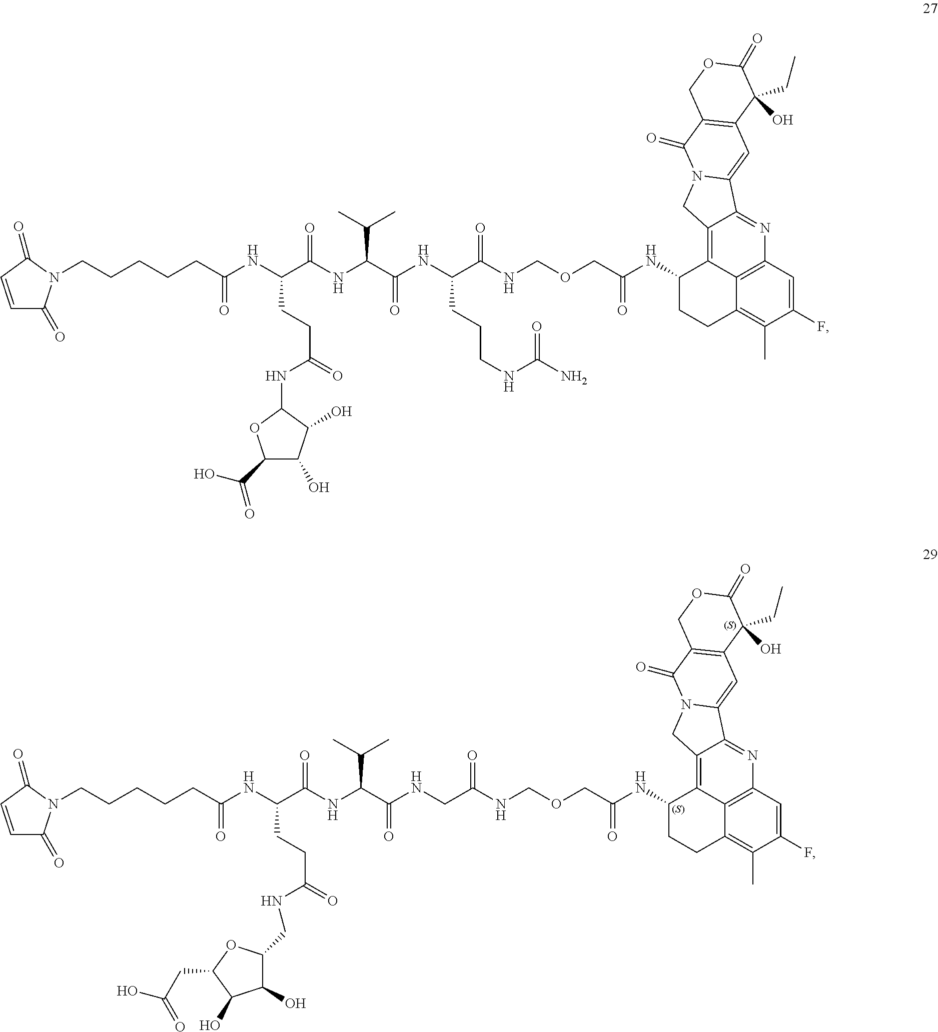 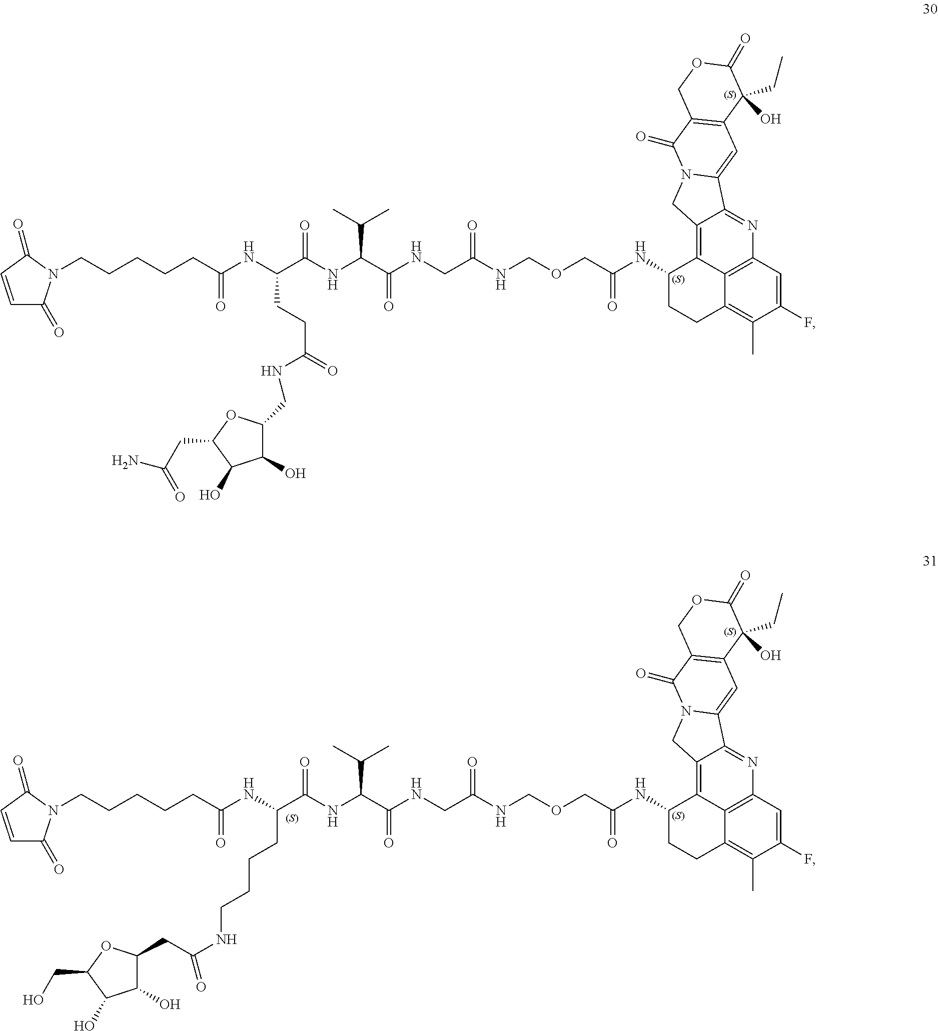 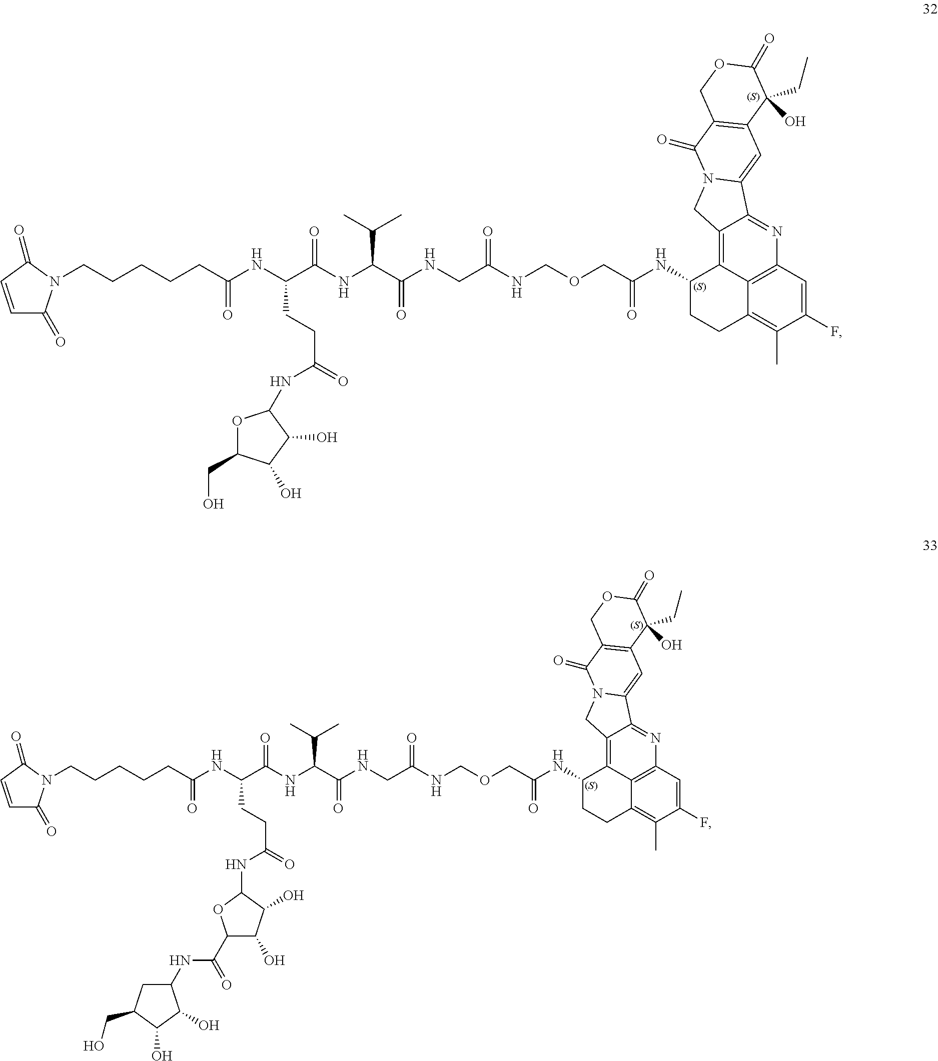 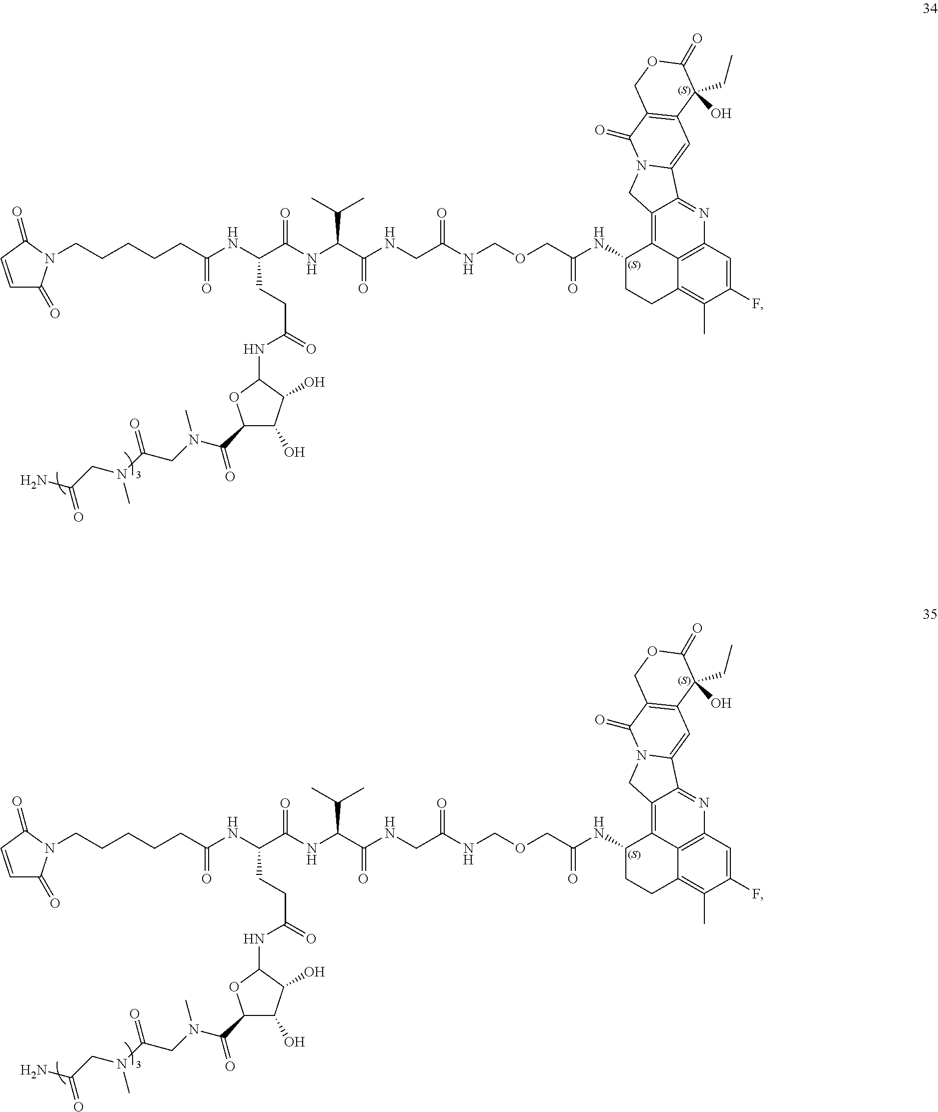 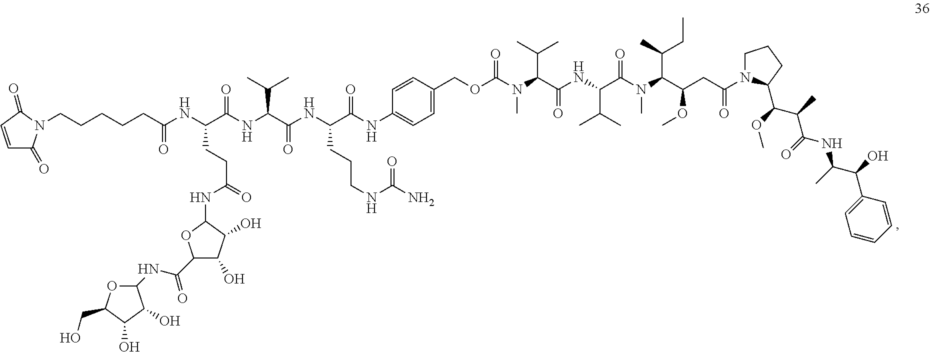 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)NC2OC(C(=O)NC3O[C@H](CO)[C@@H](O)[C@H]3O)[C@@H](O)[C@H]2O)NC(=O)CCCCCN2C(=O)C=CC2=O)C(C)C)cc1)C(C)C] 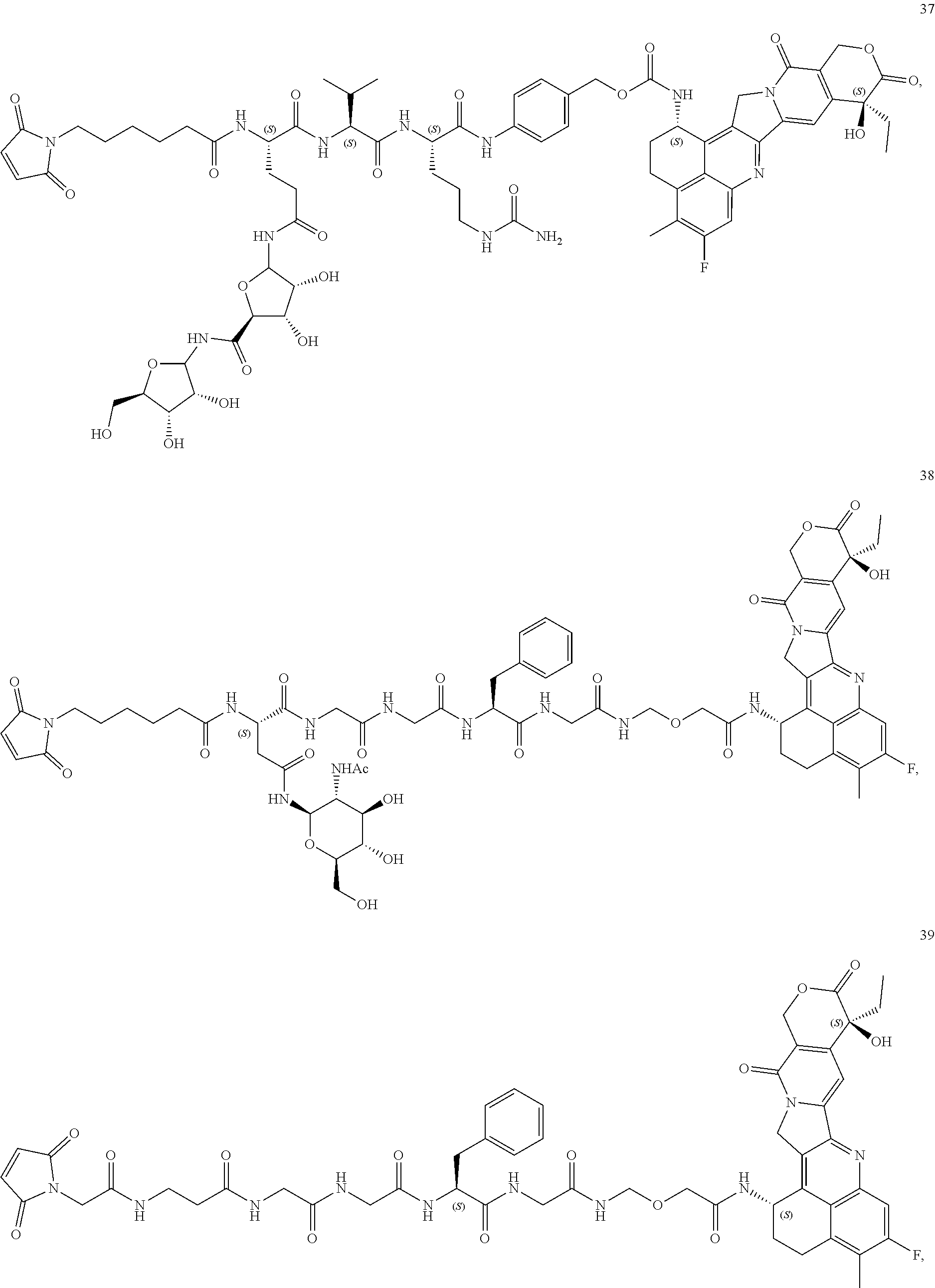 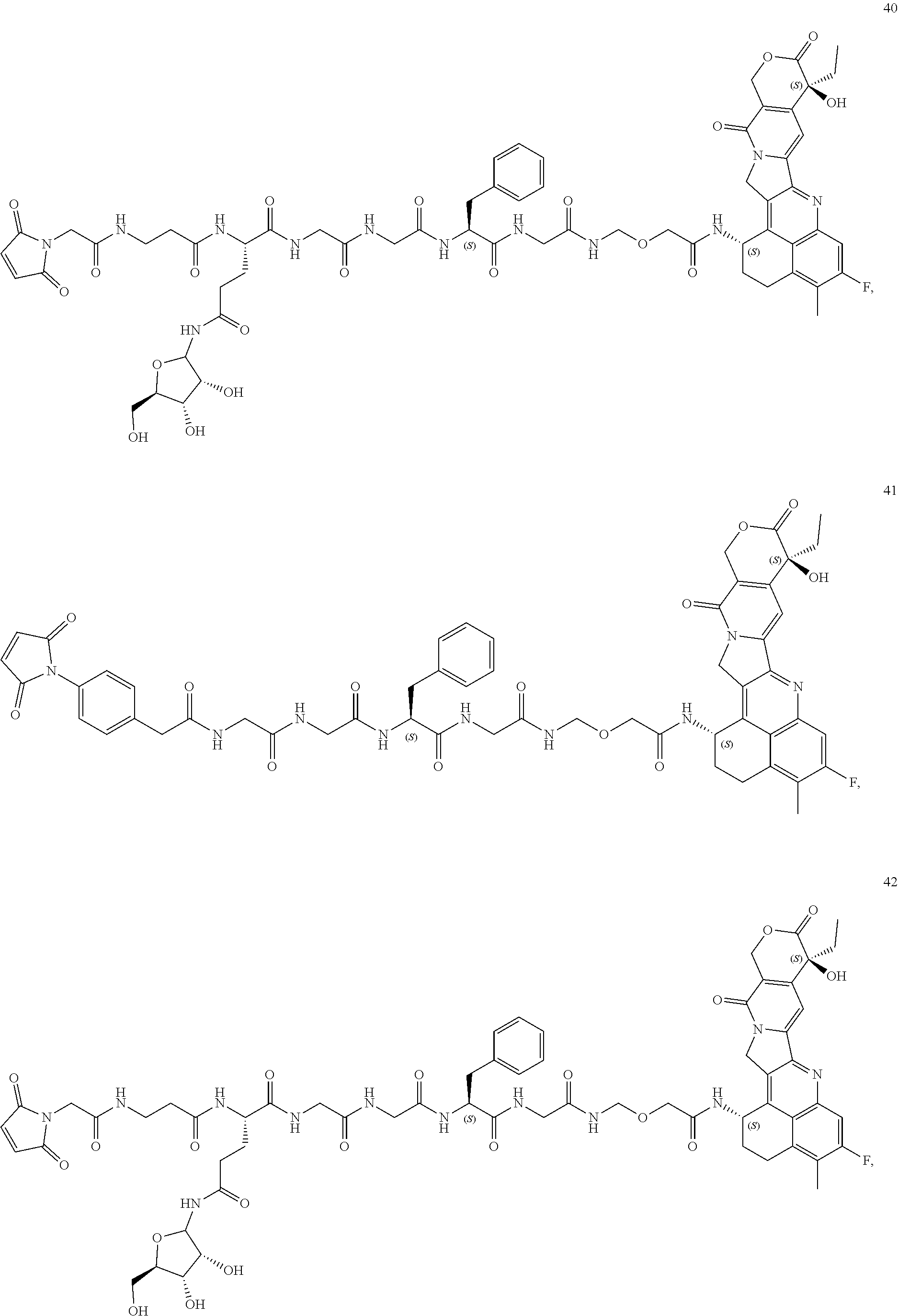 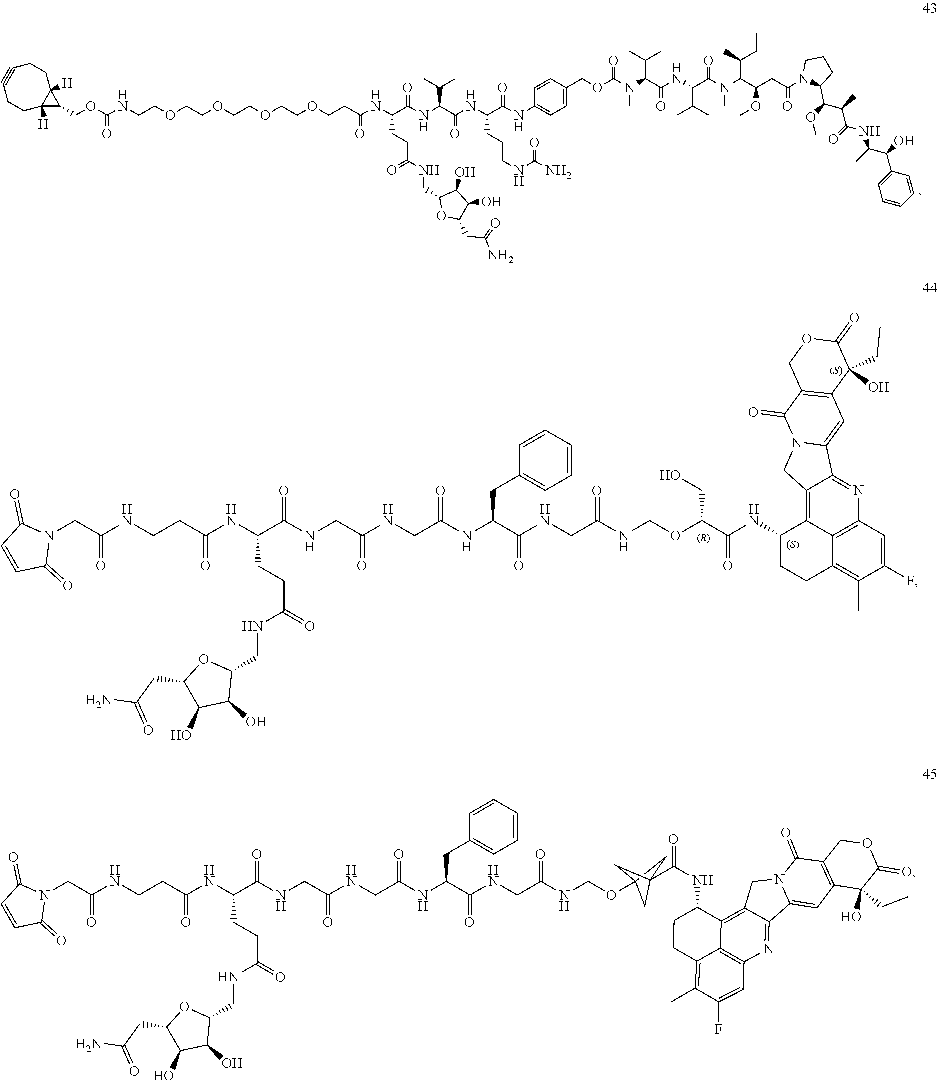 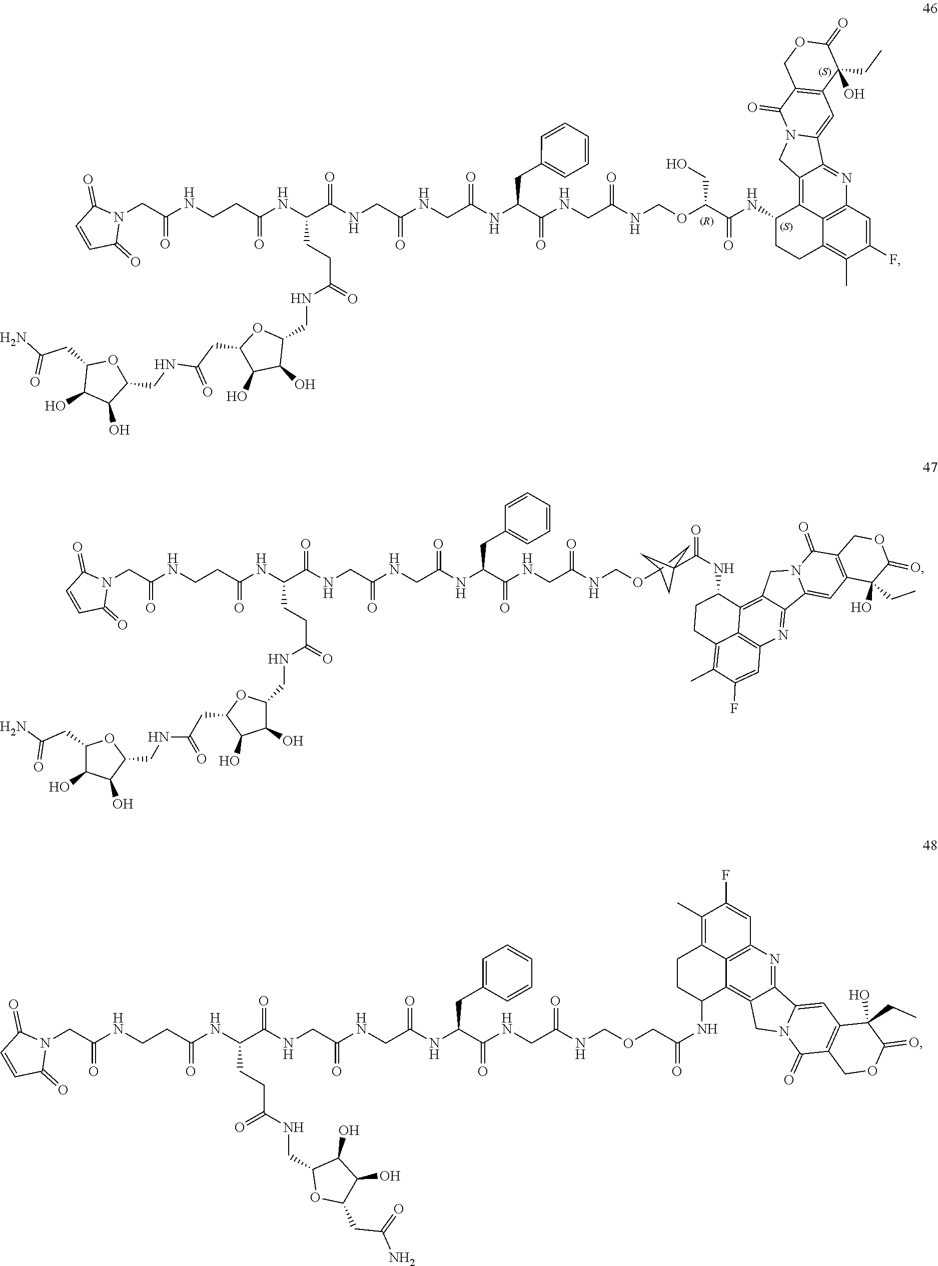 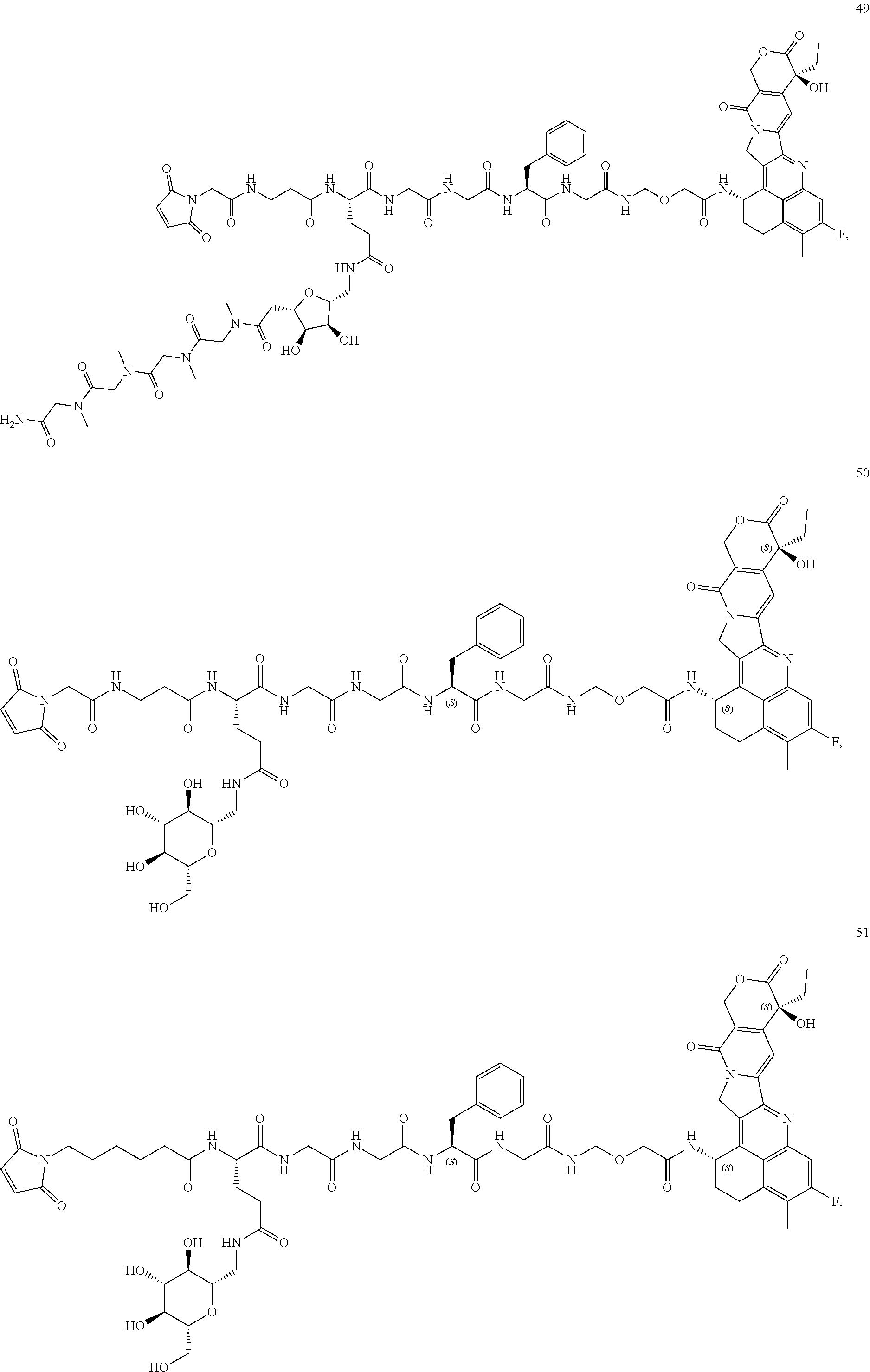 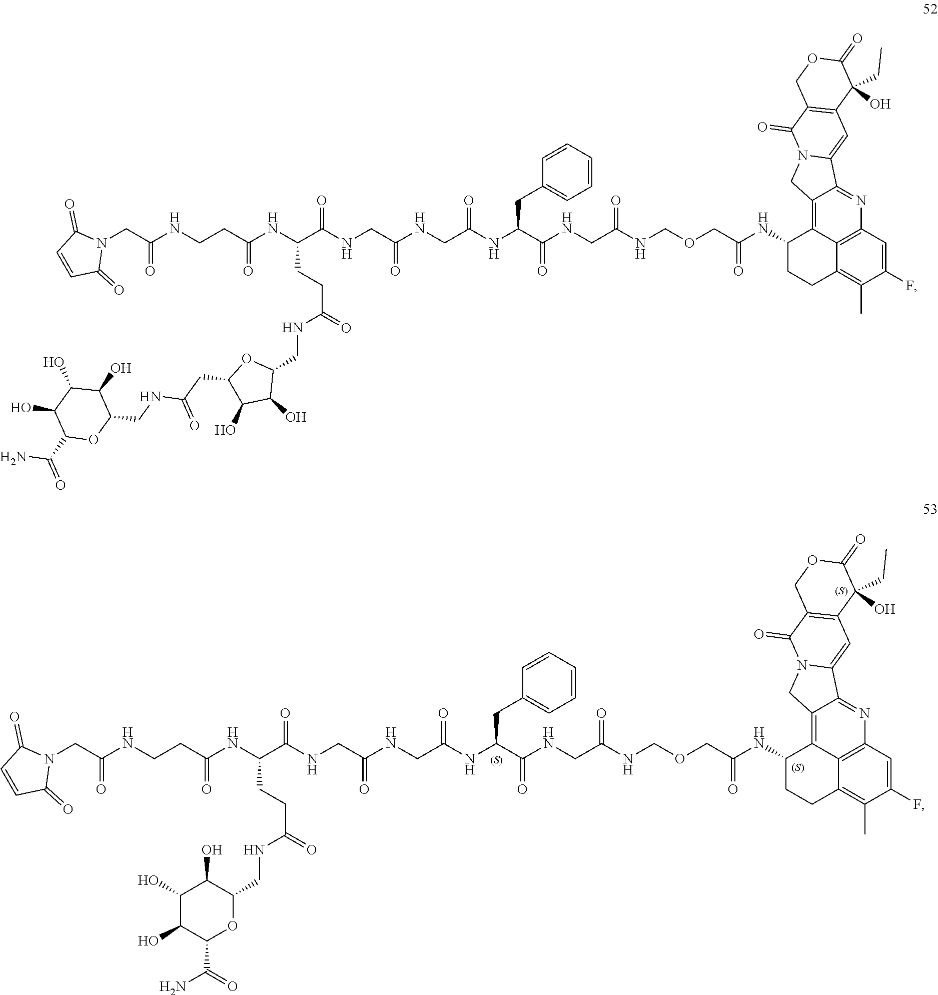 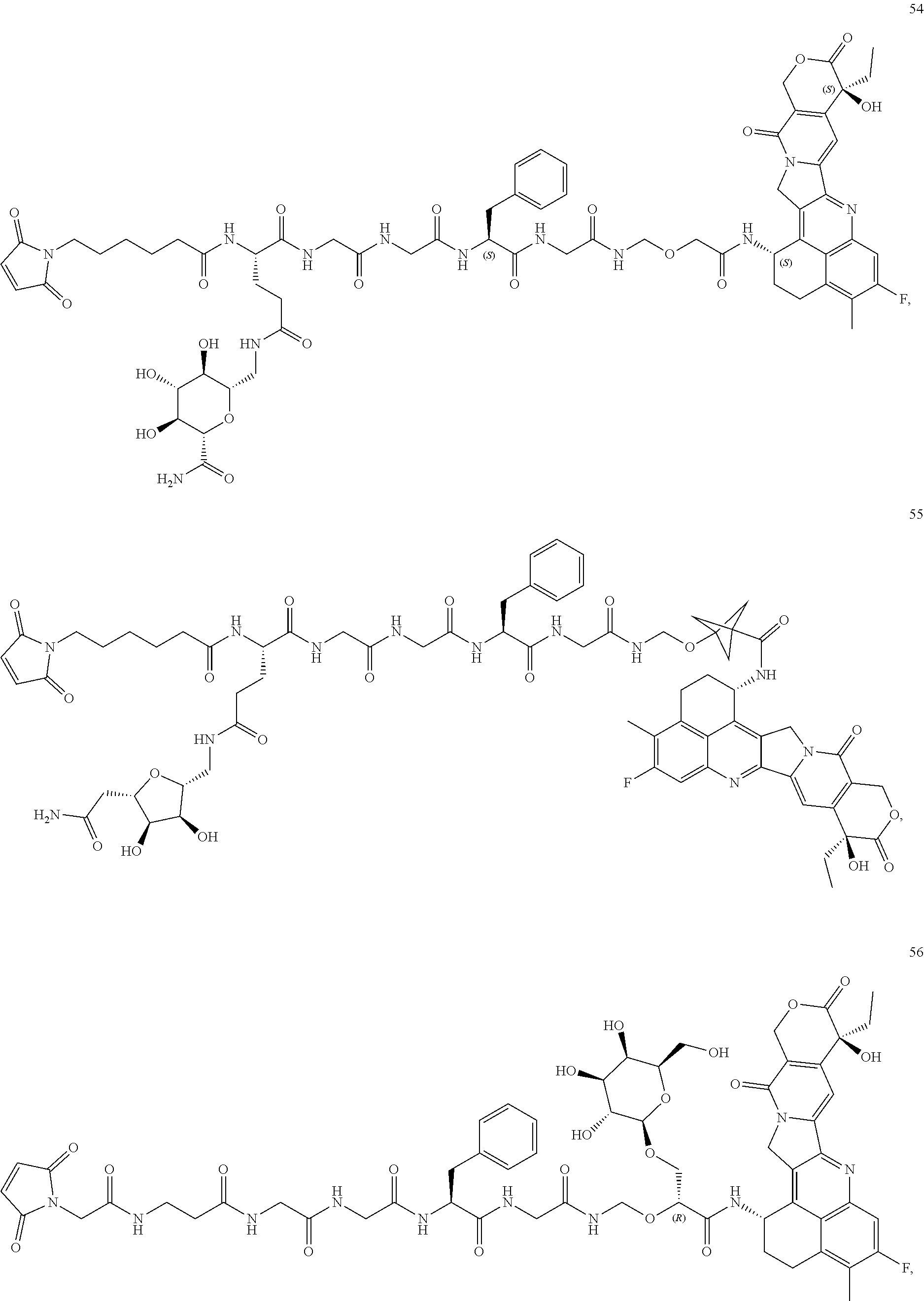 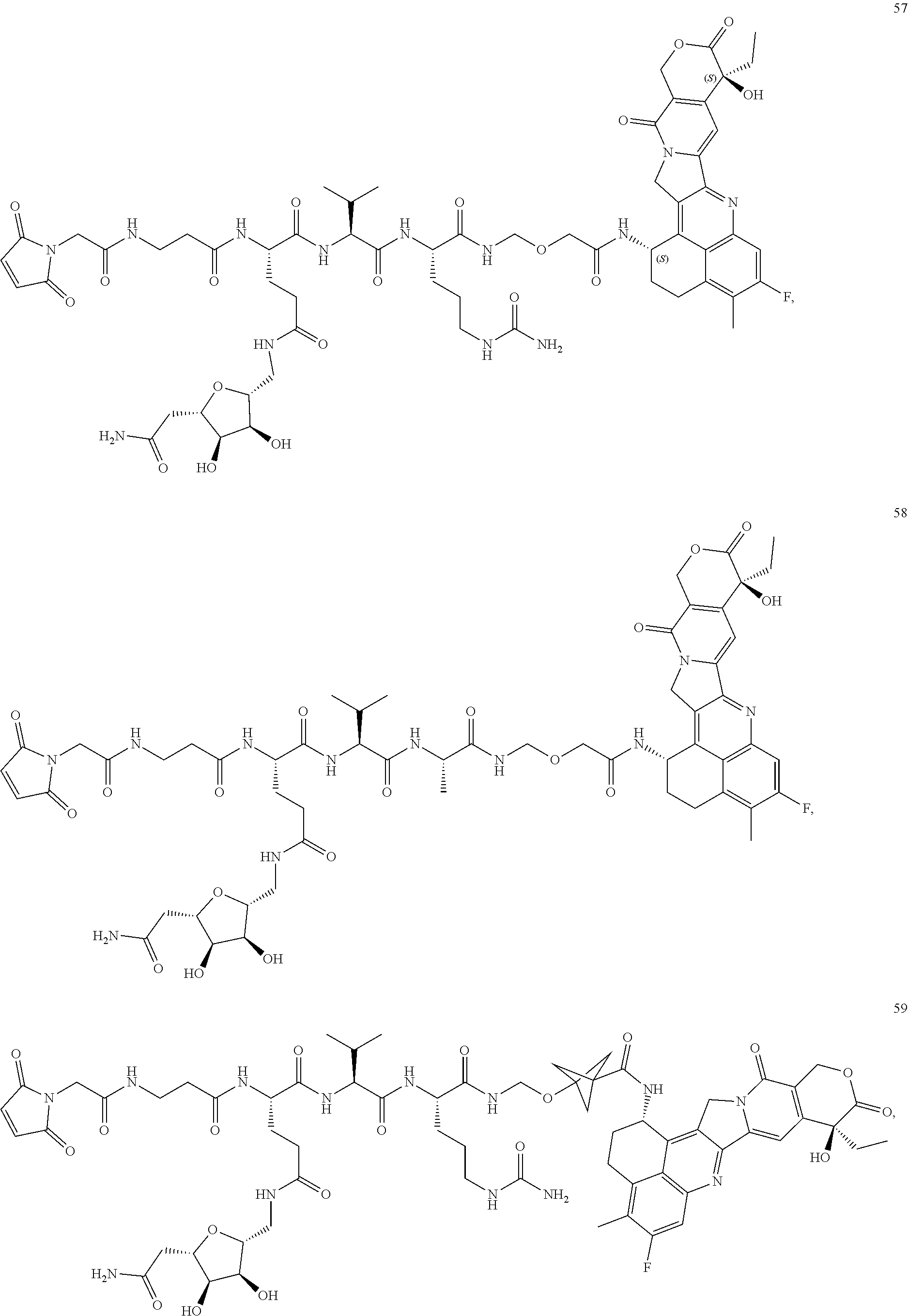 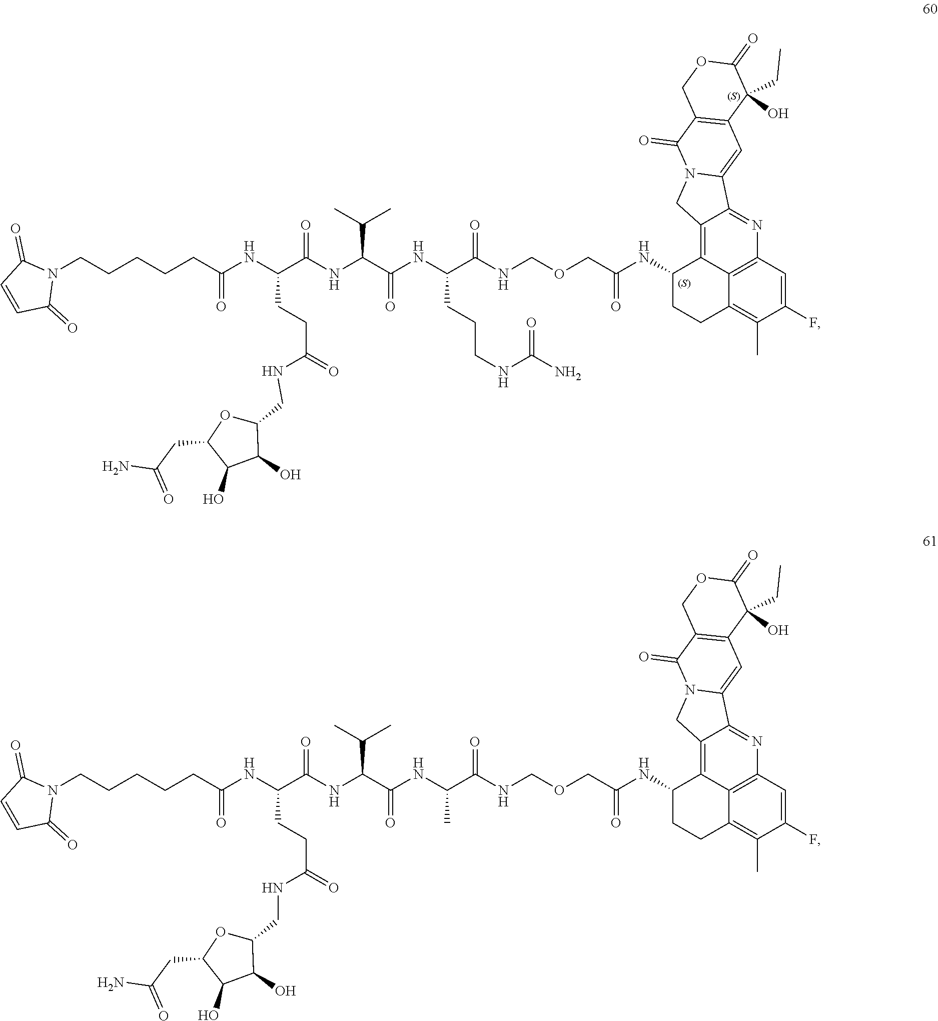 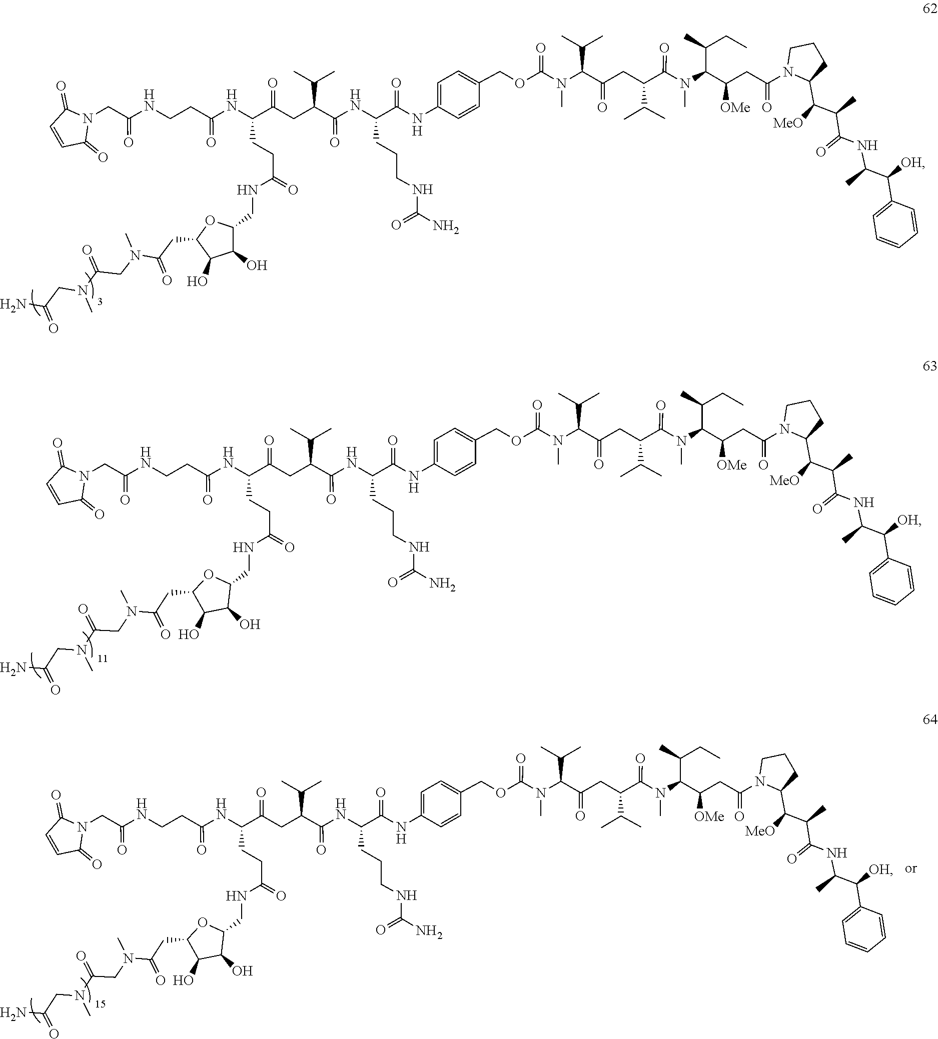 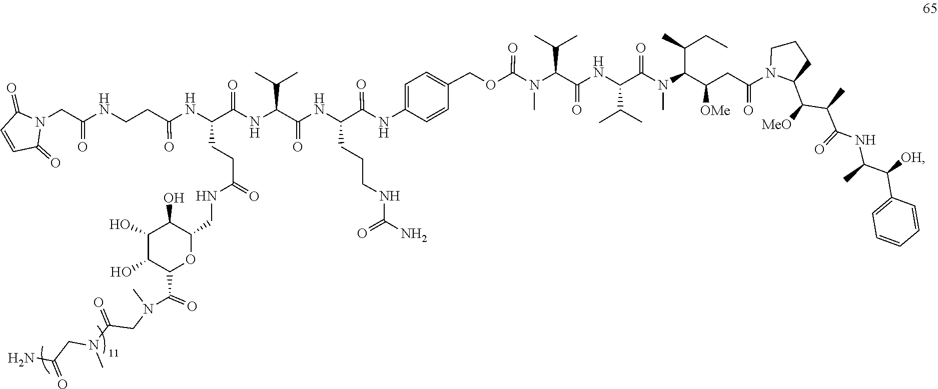
or a pharmaceutically acceptable salt thereof.
